SEQUENCE LISTING (841 sequences)

SEQ ID NO: 1 - DNA, 141 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 2 - DNA, 130 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgcccgggcggcctcagtgagcgagcgagcgcgcagctgcctgcagg

SEQ ID NO: 3 - DNA, 1923 bp, Artificial Sequence
  Description of Artificial Sequence: Synthetic polynucleotide
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttttagccttcttctttttcctacagctcctgggcaacgtgctggttattgtgctgtctcatcatttgtcgacagaattcctcgaagatccgaaggggttcaagcttggcattccggtactgttggtaaagcca

SEQ ID NO: 4 - DNA, 1272 bp, Artificial Sequence
  Description of Artificial Sequence: Synthetic polynucleotide
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccgggttcaaaaccacttgctgggtggggagtcgtcagtaagtggctatgccccgaccccgaagcctgtttccccatctgtacaatggaaatgataaagacgcccatctgatagggtttttgtggcaaataaacatttggtttttttgttttgttttgttttgttttttgagatggaggtttgctctgtcgcccaggctggagtgcagtgacacaatctcatctcaccacaaccttcccctgcctcagcctcccaagtagctgggattacaagcatgtgccaccacacctggctaattttctatttttagtagagacgggtttctccatgttggtcagcctcagcctcccaagtaactgggattacaggcctgtgccaccacacccggctaattttttctatttttgacagggacggggtttcaccatgttggtcaggctggtctagaggtaccggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactga

SEQ ID NO: 5 - DNA, 547 bp, Artificial Sequence
  Description of Artificial Sequence: Synthetic polynucleotide
ccctaaaatgggcaaacattgcaagcagcaaacagcaaacacacagccctccctgcctgctgaccttggagctggggcagaggtcagagacctctctgggcccatgccacctccaacatccactcgaccccttggaatttttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactga

SEQ ID NO: 6 - DNA, 1179 bp, Artificial Sequence
  Description of Artificial Sequence: Synthetic polynucleotide
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 7 - DNA, 8 bp, Artificial Sequence
  Description of Artificial Sequence: Synthetic oligonucleotide
gtttaaac

SEQ ID NO: 8 - DNA, 581 bp, Artificial Sequence
  Description of Artificial Sequence: Synthetic polynucleotide
gagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 9 - DNA, 225 bp, Artificial Sequence
  Description of Artificial Sequence: Synthetic polynucleotide
tgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggc

SEQ ID NO: 10 - DNA, 213 bp, Artificial Sequence
  Description of Artificial Sequence: Synthetic polynucleotide
taagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 11 - DNA, 1260 bp, Adeno-associated virus - 2
atggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctga

SEQ ID NO: 12 - DNA, 1932 bp, Adeno-associated virus - 2
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctga

SEQ ID NO: 13 - DNA, 1876 bp, Artificial Sequence
  Description of Artificial Sequence: Synthetic polynucleotide
cgcagccaccatggcggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 14 - DNA, 1194 bp, Artificial Sequence
  Description of Artificial Sequence: Synthetic polynucleotide
atggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagaccgctaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 15 - DNA, 141 bp, Artificial Sequence
  Description of Artificial Sequence: Synthetic polynucleotide
aataaacgataacgccgttggtggcgtgaggcatgtaaaaggttacatcattatcttgttcgccatccggttggtataaatagacgttcatgttggtttttgtttcagttgcaagttggctgcggcgcgcgcagcaccttt

SEQ ID NO: 16 - DNA, 556 bp, Artificial Sequence
  Description of Artificial Sequence: Synthetic polynucleotide
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacactcgagggccctgtctcctcagcttcaggcaccaccactgacctgggacagtgaatccggacatcgattctaaggtaaatataaaatttttaagtgtataatttgttaaactactgattctaattgtttctctcttttagattccaacctttggaactga

SEQ ID NO: 17 - DNA, 80 bp, Artificial Sequence
  Description of Artificial Sequence: Synthetic oligonucleotide
gcgcgctcgctcgctcactgaggccgggcgaccaaaggtcgcccgacgcccgggcggcctcagtgagcgagcgagcgcgc

SEQ ID NO: 18 - DNA, 241 bp, Artificial Sequence
  Description of Artificial Sequence: Synthetic polynucleotide
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 19 - DNA, 215 bp, Artificial Sequence
  Description of Artificial Sequence: Synthetic polynucleotide
gaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcgtataagaactgtatgagaccac

SEQ ID NO: 20 - DNA, 150 bp, Artificial Sequence
  Description of Artificial Sequence: Synthetic polynucleotide
ataaacgataacgccgttggtggcgtgaggcatgtaaaaggttacatcattatcttgttcgccatccggttggtataaatagacgttcatgttggtttttgtttcagttgcaagttggctgcggcgcgcgcagcacctttgcggccatct

SEQ ID NO: 21 - DNA, 546 bp, Artificial Sequence
  Description of Artificial Sequence: Synthetic polynucleotide
ccctaaaatgggcaaacattgcaagcagcaaacagcaaacacacagccctccctgcctgctgaccttggagctggggcagaggtcagagacctctctgggcccatgccacctccaacatccactcgaccccttggaatttttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactg

SEQ ID NO: 22 - DNA, 317 bp, Artificial Sequence
  Description of Artificial Sequence: Synthetic polynucleotide
ggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaa

SEQ ID NO: 23 - DNA, 576 bp, Artificial Sequence
  Description of Artificial Sequence: Synthetic polynucleotide
tagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcag

SEQ ID NO: 24 - DNA, 1313 bp, Artificial Sequence
  Description of Artificial Sequence: Synthetic polynucleotide
ggagccgagagtaattcatacaaaaggagggatcgccttcgcaaggggagagcccagggaccgtccctaaattctcacagacccaaatccctgtagccgccccacgacagcgcgaggagcatgcgcccagggctgagcgcgggtagatcagagcacacaagctcacagtccccggcggtggggggaggggcgcgctgagcgggggccagggagctggcgcggggcaaactgggaaagtggtgtcgtgtgctggctccgccctcttcccgagggtgggggagaacggtatataagtgcggtagtcgccttggacgttctttttcgcaacgggtttgccgtcagaacgcaggtgagtggcgggtgtggcttccgcgggccccggagctggagccctgctctgagcgggccgggctgatatgcgagtgtcgtccgcagggtttagctgtgagcattcccacttcgagtggcgggcggtgcgggggtgagagtgcgaggcctagcggcaaccccgtagcctcgcctcgtgtccggcttgaggcctagcgtggtgtccgccgccgcgtgccactccggccgcactatgcgttttttgtccttgctgccctcgattgccttccagcagcatgggctaacaaagggagggtgtggggctcactcttaaggagcccatgaagcttacgttggataggaatggaagggcaggaggggcgactggggcccgcccgccttcggagcacatgtccgacgccacctggatggggcgaggcctgtggctttccgaagcaatcgggcgtgagtttagcctacctgggccatgtggccctagcactgggcacggtctggcctggcggtgccgcgttcccttgcctcccaacaagggtgaggccgtcccgcccggcaccagttgcttgcgcggaaagatggccgctcccggggccctgttgcaaggagctcaaaatggaggacgcggcagcccggtggagcgggcgggtgagtcacccacacaaaggaagagggccttgcccctcgccggccgctgcttcctgtgaccccgtggtctatcggccgcatagtcacctcgggcttctcttgagcaccgctcgtcgcggcggggggaggggatctaatggcgttggagtttgttcacatttggtgggtggagactagtcaggccagcctggcgctggaagtcattcttggaatttgcccctttgagtttggagcgaggctaattctcaagcctcttagcggttcaaaggtattttctaaacccgtttccaggtgttgtgaaagccaccgctaattcaaagcaa

SEQ ID NO: 25 - protein, 19 aa, Artificial Sequence
  Description of Artificial Sequence: Synthetic peptide
MDWTWRILFLVAAATGAHS

SEQ ID NO: 26 - protein, 19 aa, Artificial Sequence
  Description of Artificial Sequence: Synthetic peptide
MLPSQLIGFLLLWVPASRG

SEQ ID NO: 27 - protein, 7 aa, Artificial Sequence
  Description of Artificial Sequence: Synthetic peptide
PKKKRKV

SEQ ID NO: 28-44 - skipped (17 entries)

SEQ ID NO: 45 - DNA, 6 bp, Artificial Sequence
  Description of Artificial Sequence: Synthetic oligonucleotide
ggttga

SEQ ID NO: 46 - DNA, 4 bp, Artificial Sequence
  Description of Artificial Sequence: Synthetic oligonucleotide
agtt

SEQ ID NO: 47 - DNA, 6 bp, Artificial Sequence
  Description of Artificial Sequence: Synthetic oligonucleotide
ggttgg

SEQ ID NO: 48 - DNA, 6 bp, Artificial Sequence
  Description of Artificial Sequence: Synthetic oligonucleotide
agttgg

SEQ ID NO: 49 - DNA, 6 bp, Artificial Sequence
  Description of Artificial Sequence: Synthetic oligonucleotide
agttga

SEQ ID NO: 50 - DNA, 6 bp, Artificial Sequence
  Description of Artificial Sequence: Synthetic oligonucleotide
rrttrr

SEQ ID NO: 51 - DNA, 141 bp, Artificial Sequence
  Description of Artificial Sequence: Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 52 - DNA, 130 bp, Artificial Sequence
  Description of Artificial Sequence: Synthetic polynucleotide
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 53-62 - skipped (10 entries)

SEQ ID NO: 63 - DNA, 126 bp, Artificial Sequence
  Description of Artificial Sequence: Synthetic polynucleotide
cctgcaggcagctgcgcgctcgctcgctcactgaggccgcccgggaaacccgggcgtgcccgggcgcctcagtgagcgagcgagcgcgcagagagggagtggccaactccatcactaggggttcct

SEQ ID NO: 64 - DNA, 120 bp, Artificial Sequence
  Description of Artificial Sequence: Synthetic polynucleotide
aggaacccctagtgatggagttggccactccctctctgcgcgctcgctcgctcactgaggccgcccgggaaacccgggcgtgcgcctcagtgagcgagcgagcgcgcagctgcctgcagg

SEQ ID NO: 65 - skipped

SEQ ID NO: 66 - DNA, 141 bp, Artificial Sequence; identical to SEQ ID NO: 15
  Description of Artificial Sequence: Synthetic polynucleotide

SEQ ID NO: 67 - DNA, 1876 bp, Artificial Sequence
  Description of Artificial Sequence: Synthetic polynucleotide
cgcagccaccatggcggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 68 - DNA, 129 bp, Artificial Sequence
  Description of Artificial Sequence: Synthetic polynucleotide
atcatggagataattaaaatgataaccatctcgcaaataaataagtattttactgttttcgtaacagttttgtaataaaaaaacctataaatattccggattattcataccgtcccaccatcgggcgcg

SEQ ID NO: 69 - DNA, 1203 bp, Artificial Sequence
  Description of Artificial Sequence: Synthetic polynucleotide
gccgccaccatggagttggtgggctggctcgtggacaaaggcattacttcggaaaagcagtggattcaggaggatcaggcatcttacatctcattcaacgctgccagtaactcgaggtcccagatcaaggcagcgctggacaacgcgggaaagattatgagtctgaccaaaactgctccagactacctcgttggtcagcaaccggtggaagatatctccagcaacaggatctacaagattctggagctcaacggctacgaccctcaatacgctgcctcagtgttcttgggttgggccaccaagaaattcggcaagagaaacactatctggctgttcggccccgctaccactggaaagacaaacatcgcagaagcgattgctcacacggtgccattctacggctgcgtcaactggacaaacgagaacttcccgttcaacgactgtgtcgataagatggttatctggtgggaggaaggaaagatgacggccaaagtggtcgaaagcgccaaggcaattctgggtggctctaaagtgcgcgtcgaccagaagtgcaaatcttcagctcaaatcgatcctacccccgttattgtgacatcaaacacgaacatgtgtgccgtgatcgacggaaacagtacaacgttcgaacaccagcaacctctccaggatcgtatgttcaagttcgagctcacccgccgtttggaccatgatttcggcaaggtcactaaacaagaggttaaggacttcttccgctgggctaaagatcacgttgtggaggttgaacatgagttctacgtcaagaaaggaggtgctaagaaacgtccagccccgtcggacgcagatatctccgaacctaagagggtgagagagtcggtcgcacagccaagcacttctgacgcagaagcttccattaactacgcagataggtaccaaaacaagtgcagcagacacgtgggtatgaacttgatgctgttcccatgccgccagtgtgagcgtatgaaccaaaactctaacatctgtttcacacatggccagaaggactgcctcgaatgtttccctgtgtcagagagtcagcccgtctcagtcgttaagaaagcttaccaaaagttgtgctacatccaccatattatgggtaaagtccctgatgcctgtaccgcttgtgatctggtcaacgtggatttggacgactgtattttcgagcaataa

SEQ ID NO: 70-72 - skipped (3 entries)

SEQ ID NO: 73 - DNA, 225 bp, Artificial Sequence; identical to SEQ ID NO: 9
  Description of Artificial Sequence: Synthetic polynucleotide

SEQ ID NO: 74 - DNA, 1177 bp, Artificial Sequence
  Description of Artificial Sequence: Synthetic polynucleotide
ggctcagaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccgggttcaaaaccacttgctgggtggggagtcgtcagtaagtggctatgccccgaccccgaagcctgtttccccatctgtacaatggaaatgataaagacgcccatctgatagggtttttgtggcaaataaacatttggtttttttgttttgttttgttttgttttttgagatggaggtttgctctgtcgcccaggctggagtgcagtgacacaatctcatctcaccacaaccttcccctgcctcagcctcccaagtagctgggattacaagcatgtgccaccacacctggctaattttctatttttagtagagacgggtttctccatgttggtcagcctcagcctcccaagtaactgggattacaggcctgtgccaccacacccggctaattttttctatttttgacagggacggggtttcaccatgttggtcaggctggtctagaggtaccggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 75-94 - skipped (20 entries)

SEQ ID NO: 95 - DNA, 122 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacggcctcagtgagcgagcgagcgcgcagctgcctgcagg

SEQ ID NO: 96 - DNA, 72 bp, Artificial Sequence
  Description of Artificial Sequence: Synthetic oligonucleotide
gcgcgctcgctcgctcactgaggccgggcgaccaaaggtcgcccgacggcctcagtgagcgagcgagcgcgc

SEQ ID NO: 97 - DNA, 80 bp, Artificial Sequence; identical to SEQ ID NO: 17
  Description of Artificial Sequence: Synthetic oligonucleotide

SEQ ID NO: 98 - DNA, 72 bp, Artificial Sequence; identical to SEQ ID NO: 96
  Description of Artificial Sequence: Synthetic oligonucleotide

SEQ ID NO: 99 - DNA, 122 bp, Artificial Sequence; identical to SEQ ID NO: 95
  Description of Artificial Sequence: Synthetic polynucleotide

SEQ ID NO: 100 - DNA, 130 bp, Artificial Sequence; identical to SEQ ID NO: 2
  Description of Artificial Sequence: Synthetic polynucleotide

SEQ ID NO: 101 - DNA, 70 bp, Artificial Sequence
  Description of Artificial Sequence: Synthetic oligonucleotide
gcgcgctcgctcgctcactgaggccgcccgggaaacccgggcgtgcgcctcagtgagcgagcgagcgcgc

SEQ ID NO: 102 - DNA, 70 bp, Artificial Sequence
  Description of Artificial Sequence: Synthetic oligonucleotide
gcgcgctcgctcgctcactgaggcgcacgcccgggtttcccgggcggcctcagtgagcgagcgagcgcgc

SEQ ID NO: 103 - DNA, 72 bp, Artificial Sequence
  Description of Artificial Sequence: Synthetic oligonucleotide
gcgcgctcgctcgctcactgaggccgtcgggcgacctttggtcgcccggcctcagtgagcgagcgagcgcgc

SEQ ID NO: 104 - DNA, 72 bp, Artificial Sequence; identical to SEQ ID NO: 96
  Description of Artificial Sequence: Synthetic oligonucleotide

SEQ ID NO: 105 - DNA, 72 bp, Artificial Sequence
  Description of Artificial Sequence: Synthetic oligonucleotide
gcgcgctcgctcgctcactgaggccgcccgggcaaagcccgggcgtcggcctcagtgagcgagcgagcgcgc

SEQ ID NO: 106 - DNA, 72 bp, Artificial Sequence
  Description of Artificial Sequence: Synthetic oligonucleotide
gcgcgctcgctcgctcactgaggccgacgcccgggctttgcccgggcggcctcagtgagcgagcgagcgcgc

SEQ ID NO: 107 - DNA, 83 bp, Artificial Sequence
  Description of Artificial Sequence: Synthetic oligonucleotide
gcgcgctcgctcgctcactgaggccgcccgggcaaagcccgggcgtcgggctttgcccggcctcagtgagcgagcgagcgcgc

SEQ ID NO: 108 - DNA, 83 bp, Artificial Sequence
  Description of Artificial Sequence: Synthetic oligonucleotide
gcgcgctcgctcgctcactgaggccgggcaaagcccgacgcccgggctttgcccgggcggcctcagtgagcgagcgagcgcgc

SEQ ID NO: 109 - DNA, 77 bp, Artificial Sequence
  Description of Artificial Sequence: Synthetic oligonucleotide
gcgcgctcgctcgctcactgaggccgaaacgtcgggcgacctttggtcgcccggcctcagtgagcgagcgagcgcgc

SEQ ID NO: 110 - DNA, 77 bp, Artificial Sequence
  Description of Artificial Sequence: Synthetic oligonucleotide
gcgcgctcgctcgctcactgaggccgggcgaccaaaggtcgcccgacgtttcggcctcagtgagcgagcgagcgcgc

SEQ ID NO: 111 - DNA, 51 bp, Artificial Sequence
  Description of Artificial Sequence: Synthetic oligonucleotide
gcgcgctcgctcgctcactgaggcaaagcctcagtgagcgagcgagcgcgc

SEQ ID NO: 112 - DNA, 51 bp, Artificial Sequence
  Description of Artificial Sequence: Synthetic oligonucleotide
gcgcgctcgctcgctcactgaggctttgcctcagtgagcgagcgagcgcgc

SEQ ID NO: 113 - DNA, 80 bp, Artificial Sequence
  Description of Artificial Sequence: Synthetic oligonucleotide
gcgcgctcgctcgctcactgaggccgcccgggcgtcgggcgacctttggtcgcccggcctcagtgagcgagcgagcgcgc

SEQ ID NO: 114 - DNA, 80 bp, Artificial Sequence; identical to SEQ ID NO: 17
  Description of Artificial Sequence: Synthetic oligonucleotide

SEQ ID NO: 115 - skipped

SEQ ID NO: 116 - DNA, 79 bp, Artificial Sequence
  Description of Artificial Sequence: Synthetic oligonucleotide
gcgcgctcgctcgctcactgaggccgggcgaccaaaggtcgcccgacgcccgggcgcctcagtgagcgagcgagcgcgc

SEQ ID NO: 117 - DNA, 89 bp, Artificial Sequence
  Description of Artificial Sequence: Synthetic oligonucleotide
gcgcgctcgctcgctcactgaggccgcccgggcaaagcccgggcgtcgggcgactttgtcgcccggcctcagtgagcgagcgagcgcgc

SEQ ID NO: 118 - DNA, 89 bp, Artificial Sequence
  Description of Artificial Sequence: Synthetic oligonucleotide
gcgcgctcgctcgctcactgaggccgggcgacaaagtcgcccgacgcccgggctttgcccgggcggcctcagtgagcgagcgagcgcgc

SEQ ID NO: 119 - DNA, 87 bp, Artificial Sequence
  Description of Artificial Sequence: Synthetic oligonucleotide
gcgcgctcgctcgctcactgaggccgcccgggcaaagcccgggcgtcgggcgattttcgcccggcctcagtgagcgagcgagcgcgc

SEQ ID NO: 120 - DNA, 87 bp, Artificial Sequence
  Description of Artificial Sequence: Synthetic oligonucleotide
gcgcgctcgctcgctcactgaggccgggcgaaaatcgcccgacgcccgggctttgcccgggcggcctcagtgagcgagcgagcgcgc

SEQ ID NO: 121 - DNA, 85 bp, Artificial Sequence
  Description of Artificial Sequence: Synthetic oligonucleotide
gcgcgctcgctcgctcactgaggccgcccgggcaaagcccgggcgtcgggcgtttcgcccggcctcagtgagcgagcgagcgcgc

SEQ ID NO: 122 - DNA, 85 bp, Artificial Sequence
  Description of Artificial Sequence: Synthetic oligonucleotide
gcgcgctcgctcgctcactgaggccgggcgaaacgcccgacgcccgggctttgcccgggcggcctcagtgagcgagcgagcgcgc

SEQ ID NO: 123 - DNA, 89 bp, Artificial Sequence
  Description of Artificial Sequence: Synthetic oligonucleotide
gcgcgctcgctcgctcactgaggccgcccgggaaacccgggcgtcgggcgacctttggtcgcccggcctcagtgagcgagcgagcgcgc

SEQ ID NO: 124 - DNA, 89 bp, Artificial Sequence
  Description of Artificial Sequence: Synthetic oligonucleotide
gcgcgctcgctcgctcactgaggccgggcgaccaaaggtcgcccgacgcccgggtttcccgggcggcctcagtgagcgagcgagcgcgc

SEQ ID NO: 125 - DNA, 87 bp, Artificial Sequence
  Description of Artificial Sequence: Synthetic oligonucleotide
gcgcgctcgctcgctcactgaggccgcccggaaaccgggcgtcgggcgacctttggtcgcccggcctcagtgagcgagcgagcgcgc

SEQ ID NO: 126 - DNA, 87 bp, Artificial Sequence
  Description of Artificial Sequence: Synthetic oligonucleotide
gcgcgctcgctcgctcactgaggccgggcgaccaaaggtcgcccgacgcccggtttccgggcggcctcagtgagcgagcgagcgcgc

SEQ ID NO: 127 - DNA, 85 bp, Artificial Sequence
  Description of Artificial Sequence: Synthetic oligonucleotide
gcgcgctcgctcgctcactgaggccgcccgaaacgggcgtcgggcgacctttggtcgcccggcctcagtgagcgagcgagcgcgc

SEQ ID NO: 128 - DNA, 85 bp, Artificial Sequence
  Description of Artificial Sequence: Synthetic oligonucleotide
gcgcgctcgctcgctcactgaggccgggcgaccaaaggtcgcccgacgcccgtttcgggcggcctcagtgagcgagcgagcgcgc

SEQ ID NO: 129 - DNA, 83 bp, Artificial Sequence
  Description of Artificial Sequence: Synthetic oligonucleotide
gcgcgctcgctcgctcactgaggccgcccaaagggcgtcgggcgacctttggtcgcccggcctcagtgagcgagcgagcgcgc

SEQ ID NO: 130 - DNA, 83 bp, Artificial Sequence
  Description of Artificial Sequence: Synthetic oligonucleotide
gcgcgctcgctcgctcactgaggccgggcgaccaaaggtcgcccgacgccctttgggcggcctcagtgagcgagcgagcgcgc

SEQ ID NO: 131 - DNA, 81 bp, Artificial Sequence
  Description of Artificial Sequence: Synthetic oligonucleotide
gcgcgctcgctcgctcactgaggccgccaaaggcgtcgggcgacctttggtcgcccggcctcagtgagcgagcgagcgcgc

SEQ ID NO: 132 - DNA, 81 bp, Artificial Sequence
  Description of Artificial Sequence: Synthetic oligonucleotide
gcgcgctcgctcgctcactgaggccgggcgaccaaaggtcgcccgacgcctttggcggcctcagtgagcgagcgagcgcgc

SEQ ID NO: 133 - DNA, 79 bp, Artificial Sequence
  Description of Artificial Sequence: Synthetic oligonucleotide
gcgcgctcgctcgctcactgaggccgcaaagcgtcgggcgacctttggtcgcccggcctcagtgagcgagcgagcgcgc

SEQ ID NO: 134 - DNA, 79 bp, Artificial Sequence
  Description of Artificial Sequence: Synthetic oligonucleotide
gcgcgctcgctcgctcactgaggccgggcgaccaaaggtcgcccgacgctttgcggcctcagtgagcgagcgagcgcgc

SEQ ID NO: 135-468 - skipped (334 entries)

SEQ ID NO: 469 - DNA, 120 bp, Artificial Sequence
  Description of Artificial Sequence: Synthetic polynucleotide
aggaacccctagtgatggagttggccactccctctctgcgcgctcgctcgctcactgaggcgcacgcccgggtttcccgggcggcctcagtgagcgagcgagcgcgcagctgcctgcagg

SEQ ID NO: 470 - DNA, 122 bp, Artificial Sequence
  Description of Artificial Sequence: Synthetic polynucleotide
aggaacccctagtgatggagttggccactccctctctgcgcgctcgctcgctcactgaggccgacgcccgggctttgcccgggcggcctcagtgagcgagcgagcgcgcagctgcctgcagg

SEQ ID NO: 471 - DNA, 129 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgcccgggcgcctcagtgagcgagcgagcgcgcagctgcctgcagg

SEQ ID NO: 472 - DNA, 101 bp, Artificial Sequence
  Description of Artificial Sequence: Synthetic polynucleotide
aggaacccctagtgatggagttggccactccctctctgcgcgctcgctcgctcactgaggctttgcctcagtgagcgagcgagcgcgcagctgcctgcagg

SEQ ID NO: 473 - DNA, 139 bp, Artificial Sequence
  Description of Artificial Sequence: Synthetic polynucleotide
aggaacccctagtgatggagttggccactccctctctgcgcgctcgctcgctcactgaggccgggcgacaaagtcgcccgacgcccgggctttgcccgggcggcctcagtgagcgagcgagcgcgcagctgcctgcagg

SEQ ID NO: 474 - DNA, 137 bp, Artificial Sequence
  Description of Artificial Sequence: Synthetic polynucleotide
aggaacccctagtgatggagttggccactccctctctgcgcgctcgctcgctcactgaggccgggcgaaaatcgcccgacgcccgggctttgcccgggcggcctcagtgagcgagcgagcgcgcagctgcctgcagg

SEQ ID NO: 475 - DNA, 135 bp, Artificial Sequence
  Description of Artificial Sequence: Synthetic polynucleotide
aggaacccctagtgatggagttggccactccctctctgcgcgctcgctcgctcactgaggccgggcgaaacgcccgacgcccgggctttgcccgggcggcctcagtgagcgagcgagcgcgcagctgcctgcagg

SEQ ID NO: 476 - DNA, 133 bp, Artificial Sequence
  Description of Artificial Sequence: Synthetic polynucleotide
aggaacccctagtgatggagttggccactccctctctgcgcgctcgctcgctcactgaggccgggcaaagcccgacgcccgggctttgcccgggcggcctcagtgagcgagcgagcgcgcagctgcctgcagg

SEQ ID NO: 477 - DNA, 139 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgcccgggtttcccgggcggcctcagtgagcgagcgagcgcgcagctgcctgcagg

SEQ ID NO: 478 - DNA, 137 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgcccggtttccgggcggcctcagtgagcgagcgagcgcgcagctgcctgcagg

SEQ ID NO: 479 - DNA, 135 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgcccgtttcgggcggcctcagtgagcgagcgagcgcgcagctgcctgcagg

SEQ ID NO: 480 - DNA, 133 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgccctttgggcggcctcagtgagcgagcgagcgcgcagctgcctgcagg

SEQ ID NO: 481 - DNA, 131 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgcctttggcggcctcagtgagcgagcgagcgcgcagctgcctgcagg

SEQ ID NO: 482 - DNA, 129 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgctttgcggcctcagtgagcgagcgagcgcgcagctgcctgcagg

SEQ ID NO: 483 - DNA, 127 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgtttcggcctcagtgagcgagcgagcgcgcagctgcctgcagg

SEQ ID NO: 484 - DNA, 120 bp, Artificial Sequence
  Description of Artificial Sequence: Synthetic polynucleotide
cctgcaggcagctgcgcgctcgctcgctcactgaggccgcccgggaaacccgggcgtgcgcctcagtgagcgagcgagcgcgcagagagggagtggccaactccatcactaggggttcct

SEQ ID NO: 485 - DNA, 122 bp, Artificial Sequence
  Description of Artificial Sequence: Synthetic polynucleotide
cctgcaggcagctgcgcgctcgctcgctcactgaggccgtcgggcgacctttggtcgcccggcctcagtgagcgagcgagcgcgcagagagggagtggccaactccatcactaggggttcct

SEQ ID NO: 486 - DNA, 122 bp, Artificial Sequence
  Description of Artificial Sequence: Synthetic polynucleotide
cctgcaggcagctgcgcgctcgctcgctcactgaggccgcccgggcaaagcccgggcgtcggcctcagtgagcgagcgagcgcgcagagagggagtggccaactccatcactaggggttcct

SEQ ID NO: 487 - DNA, 129 bp, Artificial Sequence
  Description of Artificial Sequence: Synthetic polynucleotide
cctgcaggcagctgcgcgctcgctcgctcactgaggcgcccgggcgtcgggcgacctttggtcgcccggcctcagtgagcgagcgagcgcgcagagagggagtggccaactccatcactaggggttcct

SEQ ID NO: 488 - DNA, 101 bp, Artificial Sequence
  Description of Artificial Sequence: Synthetic polynucleotide
cctgcaggcagctgcgcgctcgctcgctcactgaggcaaagcctcagtgagcgagcgagcgcgcagagagggagtggccaactccatcactaggggttcct

SEQ ID NO: 489 - DNA, 139 bp, Artificial Sequence
  Description of Artificial Sequence: Synthetic polynucleotide
cctgcaggcagctgcgcgctcgctcgctcactgaggccgcccgggcaaagcccgggcgtcgggcgactttgtcgcccggcctcagtgagcgagcgagcgcgcagagagggagtggccaactccatcactaggggttcct

SEQ ID NO: 490 - DNA, 137 bp, Artificial Sequence
  Description of Artificial Sequence: Synthetic polynucleotide
cctgcaggcagctgcgcgctcgctcgctcactgaggccgcccgggcaaagcccgggcgtcgggcgattttcgcccggcctcagtgagcgagcgagcgcgcagagagggagtggccaactccatcactaggggttcct

SEQ ID NO: 491 - DNA, 135 bp, Artificial Sequence
  Description of Artificial Sequence: Synthetic polynucleotide
cctgcaggcagctgcgcgctcgctcgctcactgaggccgcccgggcaaagcccgggcgtcgggcgtttcgcccggcctcagtgagcgagcgagcgcgcagagagggagtggccaactccatcactaggggttcct

SEQ ID NO: 492 - DNA, 133 bp, Artificial Sequence
  Description of Artificial Sequence: Synthetic polynucleotide
cctgcaggcagctgcgcgctcgctcgctcactgaggccgcccgggcaaagcccgggcgtcgggctttgcccggcctcagtgagcgagcgagcgcgcagagagggagtggccaactccatcactaggggttcct

SEQ ID NO: 493 - DNA, 139 bp, Artificial Sequence
  Description of Artificial Sequence: Synthetic polynucleotide
cctgcaggcagctgcgcgctcgctcgctcactgaggccgcccgggaaacccgggcgtcgggcgacctttggtcgcccggcctcagtgagcgagcgagcgcgcagagagggagtggccaactccatcactaggggttcct

SEQ ID NO: 494 - DNA, 137 bp, Artificial Sequence
  Description of Artificial Sequence: Synthetic polynucleotide
cctgcaggcagctgcgcgctcgctcgctcactgaggccgcccggaaaccgggcgtcgggcgacctttggtcgcccggcctcagtgagcgagcgagcgcgcagagagggagtggccaactccatcactaggggttcct

SEQ ID NO: 495 - DNA, 135 bp, Artificial Sequence
  Description of Artificial Sequence: Synthetic polynucleotide
cctgcaggcagctgcgcgctcgctcgctcactgaggccgcccgaaacgggcgtcgggcgacctttggtcgcccggcctcagtgagcgagcgagcgcgcagagagggagtggccaactccatcactaggggttcct

SEQ ID NO: 496 - DNA, 133 bp, Artificial Sequence
  Description of Artificial Sequence: Synthetic polynucleotide
cctgcaggcagctgcgcgctcgctcgctcactgaggccgcccaaagggcgtcgggcgacctttggtcgcccggcctcagtgagcgagcgagcgcgcagagagggagtggccaactccatcactaggggttcct

SEQ ID NO: 497 - DNA, 131 bp, Artificial Sequence
  Description of Artificial Sequence: Synthetic polynucleotide
cctgcaggcagctgcgcgctcgctcgctcactgaggccgccaaaggcgtcgggcgacctttggtcgcccggcctcagtgagcgagcgagcgcgcagagagggagtggccaactccatcactaggggttcct

SEQ ID NO: 498 - DNA, 129 bp, Artificial Sequence
  Description of Artificial Sequence: Synthetic polynucleotide
cctgcaggcagctgcgcgctcgctcgctcactgaggccgcaaagcgtcgggcgacctttggtcgcccggcctcagtgagcgagcgagcgcgcagagagggagtggccaactccatcactaggggttcct

SEQ ID NO: 499 - DNA, 127 bp, Artificial Sequence
  Description of Artificial Sequence: Synthetic polynucleotide
cctgcaggcagctgcgcgctcgctcgctcactgaggccgaaacgtcgggcgacctttggtcgcccggcctcagtgagcgagcgagcgcgcagagagggagtggccaactccatcactaggggttcct

SEQ ID NO: 500 - DNA, 43 bp, Artificial Sequence
  Description of Artificial Sequence: Synthetic oligonucleotide
gcccgctggtttccagcgggctgcgggcccgaaacgggcccgc

SEQ ID NO: 501 - DNA, 28 bp, Artificial Sequence
  Description of Artificial Sequence: Synthetic oligonucleotide
cgggcccgtgcgggcccaaagggcccgc

SEQ ID NO: 502 - DNA, 28 bp, Artificial Sequence
  Description of Artificial Sequence: Synthetic oligonucleotide
gcccgggcacgcccgggtttcccgggcg

SEQ ID NO: 503 - DNA, 22 bp, Artificial Sequence
  Description of Artificial Sequence: Synthetic oligonucleotide
cgtgcgggcccaaagggcccgc

SEQ ID NO: 504 - DNA, 21 bp, Artificial Sequence
  Description of Artificial Sequence: Synthetic oligonucleotide
cgggcgaccaaaggtcgcccg

SEQ ID NO: 505 - DNA, 20 bp, Artificial Sequence
  Description of Artificial Sequence: Synthetic oligonucleotide
cgcccgggctttgcccgggc

SEQ ID NO: 506 - DNA, 42 bp, Artificial Sequence
  Description of Artificial Sequence: Synthetic oligonucleotide
cgggcgaccaaaggtcgcccgacgcccgggctttgcccgggc

SEQ ID NO: 507 - DNA, 21 bp, Artificial Sequence
  Description of Artificial Sequence: Synthetic oligonucleotide
cgggcgaccaaaggtcgcccg

SEQ ID NO: 508 - DNA, 20 bp, Artificial Sequence
  Description of Artificial Sequence: Synthetic oligonucleotide
cgcccgggctttgcccgggc

SEQ ID NO: 509 - DNA, 34 bp, Artificial Sequence
  Description of Artificial Sequence: Synthetic oligonucleotide
cgggcgaccaaaggtcgcccgacgcccgggcggc

SEQ ID NO: 510 - DNA, 21 bp, Artificial Sequence
  Description of Artificial Sequence: Synthetic oligonucleotide
cgggcgaccaaaggtcgcccg

SEQ ID NO: 511 - DNA, 20 bp, Artificial Sequence
  Description of Artificial Sequence: Synthetic oligonucleotide
cgcccgggctttgcccgggc

SEQ ID NO: 512 - DNA, 30 bp, Artificial Sequence
  Description of Artificial Sequence: Synthetic oligonucleotide
cggggcccgacgcccgggctttgcccgggc

SEQ ID NO: 513 - DNA, 21 bp, Artificial Sequence
  Description of Artificial Sequence: Synthetic oligonucleotide
cgggcgaccaaaggtcgcccg

SEQ ID NO: 514 - DNA, 20 bp, Artificial Sequence
  Description of Artificial Sequence: Synthetic oligonucleotide
cgcccgggctttgcccgggc

SEQ ID NO: 515 - DNA, 29 bp, Artificial Sequence
  Description of Artificial Sequence: Synthetic oligonucleotide
cgggcccgacgcccgggctttgcccgggc

SEQ ID NO: 516 - DNA, 21 bp, Artificial Sequence
  Description of Artificial Sequence: Synthetic oligonucleotide
cgggcgaccaaaggtcgcccg

SEQ ID NO: 517 - DNA, 20 bp, Artificial Sequence
  Description of Artificial Sequence: Synthetic oligonucleotide
cgcccgggctttgcccgggc

SEQ ID NO: 518 - DNA, 20 bp, Artificial Sequence
  Description of Artificial Sequence: Synthetic oligonucleotide
gcccgggcaaagcccgggcg

SEQ ID NO: 519 - DNA, 21 bp, Artificial Sequence
  Description of Artificial Sequence: Synthetic oligonucleotide
cgggcgacctttggtcgcccg

SEQ ID NO: 520 - DNA, 42 bp, Artificial Sequence
  Description of Artificial Sequence: Synthetic oligonucleotide
gcccgggcaaagcccgggcgtcgggcgacctttggtcgcccg

SEQ ID NO: 521 - DNA, 20 bp, Artificial Sequence
  Description of Artificial Sequence: Synthetic oligonucleotide
gcccgggcaaagcccgggcg

SEQ ID NO: 522 - DNA, 31 bp, Artificial Sequence
  Description of Artificial Sequence: Synthetic oligonucleotide
gcccgggcgtcgggcgacctttggtcgcccg

SEQ ID NO: 523 - DNA, 20 bp, Artificial Sequence
  Description of Artificial Sequence: Synthetic oligonucleotide
gcccgggcaaagcccgggcg

SEQ ID NO: 524 - DNA, 21 bp, Artificial Sequence
  Description of Artificial Sequence: Synthetic oligonucleotide
cgggcgacctttggtcgcccg

SEQ ID NO: 525 - DNA, 34 bp, Artificial Sequence
  Description of Artificial Sequence: Synthetic oligonucleotide
gccgcccgggcgacgggcgacctttggtcgcccg

SEQ ID NO: 526 - DNA, 20 bp, Artificial Sequence
  Description of Artificial Sequence: Synthetic oligonucleotide
gcccgggcaaagcccgggcg

SEQ ID NO: 527 - DNA, 21 bp, Artificial Sequence
  Description of Artificial Sequence: Synthetic oligonucleotide
cgggcgacctttggtcgcccg

SEQ ID NO: 528 - DNA, 31 bp, Artificial Sequence; identical to SEQ ID NO: 522
  Description of Artificial Sequence: Synthetic oligonucleotide

SEQ ID NO: 529 - DNA, 21 bp, Artificial Sequence
  Description of Artificial Sequence: Synthetic oligonucleotide
cgggcgacctttggtcgcccg

SEQ ID NO: 530 - skipped

SEQ ID NO: 531 - DNA, 16 bp, Artificial Sequence
  Description of Artificial Sequence: Synthetic oligonucleotide
gcgcgctcgctcgctc

SEQ ID NO: 532 - DNA, 8 bp, Artificial Sequence
  Description of Artificial Sequence: Synthetic oligonucleotide
actgaggc

SEQ ID NO: 533-534 - skipped (2 entries)

SEQ ID NO: 535 - DNA, 8 bp, Artificial Sequence
  Description of Artificial Sequence: Synthetic oligonucleotide
gcctcagt

SEQ ID NO: 536 - DNA, 16 bp, Artificial Sequence
  Description of Artificial Sequence: Synthetic oligonucleotide
gagcgagcgagcgcgc

SEQ ID NO: 537 - skipped

SEQ ID NO: 538 - DNA, 165 bp, Artificial Sequence
  Description of Artificial Sequence: Synthetic polynucleotide
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 539 - DNA, 140 bp, Artificial Sequence
  Description of Artificial Sequence: Synthetic polynucleotide
cccctagtgatggagttggccactccctctctgcgcgctcgctcgctcactgaggccgcccgggcaaagcccgggcgtcgggcgacctttggtcgcccggcctcagtgagcgagcgagcgcgcagagagatcactagggg

SEQ ID NO: 540 - DNA, 91 bp, Artificial Sequence
  Description of Artificial Sequence: Synthetic oligonucleotide
gcgcgctcgctcgctcactgaggccgcccgggcaaagcccgggcgtcgggcgacctttggtcgcccggcctcagtgagcgagcgagcgcgc

SEQ ID NO: 541 - DNA, 91 bp, Artificial Sequence
  Description of Artificial Sequence: Synthetic oligonucleotide
gcgcgctcgctcgctcactgaggccgggcgaccaaaggtcgcccgacgcccgggctttgcccgggcggcctcagtgagcgagcgagcgcgc

SEQ ID NO: 542 - DNA, 8 bp, Artificial Sequence
  Description of Artificial Sequence: Synthetic oligonucleotide
ttaattaa

SEQ ID NO: 543-544 - skipped (2 entries)

SEQ ID NO: 545 - DNA, 79 bp, Artificial Sequence
  Description of Artificial Sequence: Synthetic oligonucleotide
gcgcgctcgctcgctcactgaggcgcccgggcgtcgggcgacctttggtcgcccggcctcagtgagcgagcgagcgcgc

SEQ ID NO: 546 - DNA, 81 bp, Artificial Sequence
  Description of Artificial Sequence: Synthetic oligonucleotide
ctgcgcgctcgctcgctcactgaggccgggcgaccaaaggtcgcccgacgtttcggcctcagtgagcgagcgagcgcgcag

SEQ ID NO: 547 - DNA, 81 bp, Artificial Sequence
  Description of Artificial Sequence: Synthetic oligonucleotide
ctgcgcgctcgctcgctcactgaggccgaaacgtcgggcgacctttggtcgcccggcctcagtgagcgagcgagcgcgcag

SEQ ID NO: 548-549 - skipped (2 entries)

SEQ ID NO: 550 - DNA, 144 bp, Artificial Sequence
  Description of Artificial Sequence: Synthetic polynucleotide
aggaaccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 551 - DNA, 43 bp, Artificial Sequence; identical to SEQ ID NO: 500
  Description of Artificial Sequence: Synthetic oligonucleotide

SEQ ID NO: 552 - DNA, 28 bp, Artificial Sequence
  Description of Artificial Sequence: Synthetic oligonucleotide
cgggcccgtgcgggcccaaagggcccgc

SEQ ID NO: 553 - DNA, 28 bp, Artificial Sequence
  Description of Artificial Sequence: Synthetic oligonucleotide
gcccgggcacgcccgggtttcccgggcg

SEQ ID NO: 554 - DNA, 22 bp, Artificial Sequence
  Description of Artificial Sequence: Synthetic oligonucleotide
cgtgcgggcccaaagggcccgc

SEQ ID NO: 555 - DNA, 43 bp, Artificial Sequence
  Description of Artificial Sequence: Synthetic oligonucleotide
gcgggccggaaacgggcccgctgcccgctggtttccagcgggc

SEQ ID NO: 556 - DNA, 28 bp, Artificial Sequence
  Description of Artificial Sequence: Synthetic oligonucleotide
cgcccgggaaacccgggcgtgcccgggc

SEQ ID NO: 557 - DNA, 28 bp, Artificial Sequence
  Description of Artificial Sequence: Synthetic oligonucleotide
gggccgcccgggaaacccgggcgtgccc

SEQ ID NO: 558 - DNA, 29 bp, Artificial Sequence
  Description of Artificial Sequence: Synthetic oligonucleotide
  modified_base (1)..(1): a, c, t, g, unknown or other
  modified_base (3)..(3): a, c, t, g, unknown or other
  modified_base (25)..(25): a, c, t, g, unknown or other
ntntctctcttttctctctctctcncagg

SEQ ID NO: 559 - DNA, 10 bp, Artificial Sequence
  Description of Artificial Sequence: Synthetic oligonucleotide
  modified_base (1)..(1): a, c, t, g, unknown or other
naggtagagt

SEQ ID NO: 560 - DNA, 143 bp, Artificial Sequence
  Description of Artificial Sequence: Synthetic polynucleotide
ttgcccactccctctctgcgcgctcgctcgctcggtggggcctgcggaccaaaggtccgcagacggcagaggtctcctctgccggccccaccgagcgagcgacgcgcgcagagagggagtgggcaactccatcactagggtaa

SEQ ID NO: 561 - DNA, 144 bp, Artificial Sequence
  Description of Artificial Sequence: Synthetic polynucleotide
ttggccactccctctatgcgcactcgctcgctcggtggggcctggcgaccaaaggtcgccagacggacgtgggtttccacgtccggccccaccgagcgagcgagtgcgcatagagggagtggccaactccatcactagaggtat

SEQ ID NO: 562 - DNA, 127 bp, Artificial Sequence
  Description of Artificial Sequence: Synthetic polynucleotide
ttggccactccctctatgcgcgctcgctcactcactcggccctggagaccaaaggtctccagactgccggcctctggccggcagggccgagtgagtgagcgagcgcgcatagagggagtggccaact

SEQ ID NO: 563 - DNA, 166 bp, Artificial Sequence
  Description of Artificial Sequence: Synthetic polynucleotide
tcccccctgtcgcgttcgctcgctcgctggctcgtttgggggggcgacggccagagggccgtcgtctggcagctctttgagctgccacccccccaaacgagccagcgagcgagcgaacgcgacaggggggagagtgccacactctcaagcaagggggttttgtaag

SEQ ID NO: 564 - DNA, 144 bp, Artificial Sequence
  Description of Artificial Sequence: Synthetic polynucleotide
ttgcccactccctctaatgcgcgctcgctcgctcggtggggcctgcggaccaaaggtccgcagacggcagaggtctcctctgccggccccaccgagcgagcgagcgcgcatagagggagtgggcaactccatcactaggggtat

SEQ ID NO: 565 - DNA, 143 bp, Artificial Sequence
  Description of Artificial Sequence: Synthetic polynucleotide
ttaccctagtgatggagttgcccactccctctctgcgcgcgtcgctcgctcggtggggccggcagaggagacctctgccgtctgcggacctttggtccgcaggccccaccgagcgagcgagcgcgcagagagggagtgggcaa

SEQ ID NO: 566 - DNA, 144 bp, Artificial Sequence
  Description of Artificial Sequence: Synthetic polynucleotide
atacctctagtgatggagttggccactccctctatgcgcactcgctcgctcggtggggccggacgtggaaacccacgtccgtctggcgacctttggtcgccaggccccaccgagcgagcgagtgcgcatagagggagtggccaa

SEQ ID NO: 567 - DNA, 127 bp, Artificial Sequence
  Description of Artificial Sequence: Synthetic polynucleotide
agttggccacattagctatgcgcgctcgctcactcactcggccctggagaccaaaggtctccagactgccggcctctggccggcagggccgagtgagtgagcgagcgcgcatagagggagtggccaa

SEQ ID NO: 568 - DNA, 166 bp, Artificial Sequence
  Description of Artificial Sequence: Synthetic polynucleotide
cttacaaaacccccttgcttgagagtgtggcactctcccccctgtcgcgttcgctcgctcgctggctcgtttgggggggtggcagctcaaagagctgccagacgacggccctctggccgtcgcccccccaaacgagccagcgagcgagcgaacgcgacagggggga

SEQ ID NO: 569 - DNA, 144 bp, Artificial Sequence
  Description of Artificial Sequence: Synthetic polynucleotide
atacccctagtgatggagttgcccactccctctatgcgcgctcgctcgctcggtggggccggcagaggagacctctgccgtctgcggacctttggtccgcaggccccaccgagcgagcgagcgcgcattagagggagtgggcaa

SEQ ID NO: 570 - DNA, 12 bp, Artificial Sequence
  Description of Artificial Sequence: Synthetic oligonucleotide
atcgaacgatcg

SEQ ID NO: 571 - DNA, 12 bp, Artificial Sequence
  Description of Artificial Sequence: Synthetic oligonucleotide
cgatcgttcgat

SEQ ID NO: 572 - DNA, 12 bp, Artificial Sequence
  Description of Artificial Sequence: Synthetic oligonucleotide
atcgaaccatcg

SEQ ID NO: 573 - protein, 7 aa, Simian virus 40
PKKKRKV

SEQ ID NO: 574 - DNA, 21 bp, Simian virus 40
cccaagaagaagaggaaggtg

SEQ ID NO: 575 - protein, 16 aa, Unknown
  Description of Unknown: Nucleoplasmin bipartite NLS sequence
KRPAATKKAGQAKKKK

SEQ ID NO: 576 - protein, 9 aa, Unknown
  Description of Unknown: C-myc NLS sequence
PAAKRVKLD

SEQ ID NO: 577 - protein, 11 aa, Unknown
  Description of Unknown: C-myc NLS sequence
RQRRNELKRSP

SEQ ID NO: 578 - protein, 38 aa, Homo sapiens
NQSSNFGPMKGGNFGGRSSGPYGGGGQYFAKPRNQGGY

SEQ ID NO: 579 - protein, 42 aa, Unknown
  Description of Unknown: IBB domain from importin-alpha sequence
RMRIZFKNKGKDTAELRRRRVEVSVELRKAKKDEQILKRRNV

SEQ ID NO: 580 - protein, 8 aa, Unknown
  Description of Unknown: Myoma T protein sequence
VSRKRPRP

SEQ ID NO: 581 - protein, 8 aa, Unknown
  Description of Unknown: Myoma T protein sequence
PPKKARED

SEQ ID NO: 582 - protein, 8 aa, Homo sapiens
PQPKKKPL

SEQ ID NO: 583 - protein, 12 aa, Mus musculus
SALIKKKKKMAP

SEQ ID NO: 584 - protein, 5 aa, Influenza virus
DRLRR

SEQ ID NO: 585 - protein, 7 aa, Influenza virus
PKQKKRK

SEQ ID NO: 586 - protein, 10 aa, Hepatitis delta virus
RKLKKKIKKL

SEQ ID NO: 587 - protein, 10 aa, Mus musculus
REKKKFLKRR

SEQ ID NO: 588 - protein, 20 aa, Homo sapiens
KRKGDEVDGVDEVAKKKSKK

SEQ ID NO: 589 - protein, 17 aa, Homo sapiens
RKCLQAGMNLEARKTKK

SEQ ID NO: 590 - DNA, 23 bp, Artificial Sequence
  Description of Artificial Sequence: Synthetic oligonucleotide
  modified_base (1)..(20): a, c, t, or g
  modified_base (21)..(21): a, c, t, g, unknown or other
nnnnnnnnnnnnnnnnnnnnngg

SEQ ID NO: 591 - DNA, 15 bp, Artificial Sequence
  Description of Artificial Sequence: Synthetic oligonucleotide
  modified_base (1)..(12): a, c, t, or g
  modified_base (13)..(13): a, c, t, g, unknown or other
nnnnnnnnnnnnngg

SEQ ID NO: 592 - DNA, 23 bp, Artificial Sequence
  Description of Artificial Sequence: Synthetic oligonucleotide
  modified_base (1)..(20): a, c, t, or g
  modified_base (21)..(21): a, c, t, g, unknown or other
nnnnnnnnnnnnnnnnnnnnngg

SEQ ID NO: 593 - DNA, 14 bp, Artificial Sequence
  Description of Artificial Sequence: Synthetic oligonucleotide
  modified_base (1)..(11): a, c, t, or g
  modified_base (12)..(12): a, c, t, g, unknown or other
nnnnnnnnnnnngg

SEQ ID NO: 594 - DNA, 27 bp, Artificial Sequence
  Description of Artificial Sequence: Synthetic oligonucleotide
  modified_base (1)..(20): a, c, t, or g
  modified_base (21)..(22): a, c, t, g, unknown or other
nnnnnnnnnnnnnnnnnnnnnnagaaw

SEQ ID NO: 595 - DNA, 19 bp, Artificial Sequence
  Description of Artificial Sequence: Synthetic oligonucleotide
  modified_base (1)..(12): a, c, t, or g
  modified_base (13)..(14): a, c, t, g, unknown or other
nnnnnnnnnnnnnnagaaw

SEQ ID NO: 596 - DNA, 27 bp, Artificial Sequence
  Description of Artificial Sequence: Synthetic oligonucleotide
  modified_base (1)..(20): a, c, t, or g
  modified_base (21)..(22): a, c, t, g, unknown or other
nnnnnnnnnnnnnnnnnnnnnnagaaw

SEQ ID NO: 597 - DNA, 18 bp, Artificial Sequence
  Description of Artificial Sequence: Synthetic oligonucleotide
  modified_base (1)..(11): a, c, t, or g
  modified_base (12)..(13): a, c, t, g, unknown or other
nnnnnnnnnnnnnagaaw

SEQ ID NO: 598 - DNA, 25 bp, Artificial Sequence
  Description of Artificial Sequence: Synthetic oligonucleotide
  modified_base (1)..(20): a, c, t, or g
  modified_base (21)..(21): a, c, t, g, unknown or other
  modified_base (24)..(24): a, c, t, g, unknown or other
nnnnnnnnnnnnnnnnnnnnnggng

SEQ ID NO: 599 - DNA, 17 bp, Artificial Sequence
  Description of Artificial Sequence: Synthetic oligonucleotide
  modified_base (1)..(12): a, c, t, or g
  modified_base (13)..(13): a, c, t, g, unknown or other
  modified_base (16)..(16): a, c, t, g, unknown or other
nnnnnnnnnnnnnggng

SEQ ID NO: 600 - DNA, 25 bp, Artificial Sequence
  Description of Artificial Sequence: Synthetic oligonucleotide
  modified_base (1)..(20): a, c, t, or g
  modified_base (21)..(21): a, c, t, g, unknown or other
  modified_base (24)..(24): a, c, t, g, unknown or other
nnnnnnnnnnnnnnnnnnnnnggng

SEQ ID NO: 601 - DNA, 16 bp, Artificial Sequence
  Description of Artificial Sequence: Synthetic oligonucleotide
  modified_base (1)..(11): a, c, t, or g
  modified_base (12)..(12): a, c, t, or g, unknown or other
  modified_base (15)..(15): a, c, t, g, unknown or other
nnnnnnnnnnnnggng

SEQ ID NO: 602 - DNA, 137 bp, Artificial Sequence
  Description of Artificial Sequence: Synthetic polynucleotide
  modified_base (1)..(20): a, c, t, g, unknown or other
nnnnnnnnnnnnnnnnnnnngtttttgtactctcaagatttagaaataaatcttgcagaagctacaaagataaggcttcatgccgaaatcaacaccctgtcattttatggcagggtgttttcgttatttaatttttt

SEQ ID NO: 603 - DNA, 123 bp, Artificial Sequence
  Description of Artificial Sequence: Synthetic polynucleotide
  modified_base (1)..(20): a, c, t, g, unknown or other
nnnnnnnnnnnnnnnnnnnngtttttgtactctcagaaathcagaagctacaaagataaggcttcatgccgaaatcaacaccctgtcattttatggcagggtgttttcgttatttaatttttt

SEQ ID NO: 604 - DNA, 110 bp, Artificial Sequence
  Description of Artificial Sequence: Synthetic polynucleotide
  modified_base (1)..(20): a, c, t, g, unknown or other
nnnnnnnnnnnnnnnnnnnngtttttgtactctcagaaatgcagaagctacaaagataaggcttcatgccgaaatcaacaccctgtcattttatggcagggtgttttttt

SEQ ID NO: 605 - DNA, 102 bp, Artificial Sequence
  Description of Artificial Sequence: Synthetic polynucleotide
  modified_base (1)..(20): a, c, t, g, unknown or other
nnnnnnnnnnnnnnnnnnnngttttagagctagaaatagcaagttaaaataaggctagtccgttatcaacttgaaaaagtggcaccgagtcggtgctttttt

SEQ ID NO: 606 - DNA, 87 bp, Artificial Sequence
  Description of Artificial Sequence: Synthetic oligonucleotide
  modified_base (1)..(20): a, c, t, g, unknown or other
nnnnnnnnnnnnnnnnnnnngttttagagctagaaatagcaagttaaaataaggctagtccgttatcaacttgaaaaagtttttttt

SEQ ID NO: 607 - DNA, 76 bp, Artificial Sequence
  Description of Artificial Sequence: Synthetic oligonucleotide
  modified_base (1)..(20): a, c, t, g, unknown or other
nnnnnnnnnnnnnnnnnnnngttttagagctagaaatagcaagttaaaataaggctagtccgttatcatttttttt

SEQ ID NO: 608 - DNA, 28 bp, Homo sapiens
gggcagtaacggcagacttctcctcagg

SEQ ID NO: 609 - DNA, 28 bp, Homo sapiens
tggggcaaggtgaacgtggatgaagttg

SEQ ID NO: 610 - DNA, 28 bp, Homo sapiens
agagtcaggtgcaccatggtgtctgttt

SEQ ID NO: 611 - DNA, 28 bp, Homo sapiens
gtggagaagtctgccgttactgccctgt

SEQ ID NO: 612 - DNA, 28 bp, Homo sapiens
acaggagtcaggtgcaccatggtgtctg

SEQ ID NO: 613 - DNA, 28 bp, Homo sapiens
gagaagtctgccgttactgccctgtggg

SEQ ID NO: 614 - DNA, 28 bp, Homo sapiens
taacggcagacttctccacaggagtcag

SEQ ID NO: 615 - DNA, 28 bp, Homo sapiens
gccctgtggggcaaggtgaacgtggatg

SEQ ID NO: 616 - DNA, 28 bp, Homo sapiens
cacagggcagtaacggcagacttctcct

SEQ ID NO: 617 - DNA, 28 bp, Homo sapiens
ggcaaggtgaacgtggatgaagttggtg

SEQ ID NO: 618 - DNA, 28 bp, Homo sapiens
atcccatggagaggtggctgggaaggac

SEQ ID NO: 619 - DNA, 28 bp, Homo sapiens
atattgcagacaataacccctttaacct

SEQ ID NO: 620 - DNA, 28 bp, Homo sapiens
catcccaggcgtggggattagagctcca

SEQ ID NO: 621 - DNA, 28 bp, Homo sapiens
gtgcagaatatgccccgcagggtatttg

SEQ ID NO: 622 - DNA, 28 bp, Homo sapiens
gggaaggggcccagggcggtcagtgtgc

SEQ ID NO: 623 - DNA, 28 bp, Homo sapiens
acacacaggatgacttcctcaaggtggg

SEQ ID NO: 624 - DNA, 28 bp, Homo sapiens
cgccaccgggctccgggcccgagaagtt

SEQ ID NO: 625 - DNA, 28 bp, Homo sapiens
ccccagacctgcgctctggcgcccagcg

SEQ ID NO: 626 - DNA, 28 bp, Homo sapiens
ggctcgggggccggggctggagccaggg

SEQ ID NO: 627 - DNA, 28 bp, Homo sapiens
aaggcgctggcgctgcaaccggtgtacc

SEQ ID NO: 628 - DNA, 28 bp, Homo sapiens
ttgcagcgccagcgccttgggctcgggg

SEQ ID NO: 629 - DNA, 28 bp, Homo sapiens
cggtgtacccggggcccggcgccggctc

SEQ ID NO: 630 - DNA, 28 bp, Homo sapiens
ttgcattgagatagtgtggggaaggggc

SEQ ID NO: 631 - DNA, 28 bp, Homo sapiens
atctgtctgaaacggtccctggctaaac

SEQ ID NO: 632 - DNA, 28 bp, Homo sapiens
tttgcattgagatagtgtggggaagggg

SEQ ID NO: 633 - DNA, 28 bp, Homo sapiens
ctgtctgaaacggtccctggctaaactc

SEQ ID NO: 634 - DNA, 28 bp, Homo sapiens
tatttgcattgagatagtgtggggaagg

SEQ ID NO: 635 - DNA, 15 bp, Homo sapiens
cttgacaaggcaaac

SEQ ID NO: 636 - DNA, 15 bp, Homo sapiens
gtcaaggcaaggctg

SEQ ID NO: 637 - DNA, 12 bp, Homo sapiens
gatgaggatgac

SEQ ID NO: 638 - DNA, 12 bp, Homo sapiens
aaactgcaaaag

SEQ ID NO: 639 - DNA, 12 bp, Homo sapiens
gacaagcagcgg

SEQ ID NO: 640 - DNA, 13 bp, Homo sapiens
catctgctactcg

SEQ ID NO: 641 - DNA, 28 bp, Homo sapiens
atgacttgtgggtggttgtgttccagtt

SEQ ID NO: 642 - DNA, 28 bp, Homo sapiens
gggtagaagcggtcacagatatatctgt

SEQ ID NO: 643 - DNA, 28 bp, Homo sapiens
agtcagaggccaaggaagctgttggctg

SEQ ID NO: 644 - DNA, 28 bp, Homo sapiens
ttggtggcgtggacgatggccaggtagc

SEQ ID NO: 645 - DNA, 28 bp, Homo sapiens
cagttgatgccgtggcaaactggtactt

SEQ ID NO: 646 - DNA, 28 bp, Homo sapiens
ccagaagggaagcgtgatgacaaagagg

SEQ ID NO: 647 - DNA, 16 bp, Unknown
  Description of Unknown: PPP1R12C sequence
actagggacaggattg

SEQ ID NO: 648 - DNA, 16 bp, Unknown
  Description of Unknown: PPP1R12C sequence
ccccactgtggggtgg

SEQ ID NO: 649 - DNA, 28 bp, Unknown
  Description of Unknown: HPRT sequence
acccgcagtcccagcgtcgtggtgagcc

SEQ ID NO: 650 - DNA, 28 bp, Unknown
  Description of Unknown: HPRT sequence
gcatgacgggaccggtcggctcgcggca

SEQ ID NO: 651 - DNA, 28 bp, Unknown
  Description of Unknown: HPRT sequence
tgatgaaggagatgggaggccatcacat

SEQ ID NO: 652 - DNA, 28 bp, Unknown
  Description of Unknown: HPRT sequence
atctcgagcaagacgttcagtcctacag

SEQ ID NO: 653 - DNA, 28 bp, Unknown
  Description of Unknown: HPRT sequence
aagcactgaatagaaatagtgatagatc

SEQ ID NO: 654 - DNA, 28 bp, Unknown
  Description of Unknown: HPRT sequence
atgtaatccagcaggtcagcaaagaatt

SEQ ID NO: 655 - DNA, 28 bp, Unknown
  Description of Unknown: HPRT sequence
ggccggcgcgcgggctgactgctcagga

SEQ ID NO: 656 - DNA, 28 bp, Unknown
  Description of Unknown: HPRT sequence
gctccgttatggcgacccgcagccctgg

SEQ ID NO: 657 - DNA, 28 bp, Unknown
  Description of Unknown: HPRT sequence
tgcaaaaggtaggaaaaggaccaaccag

SEQ ID NO: 658 - DNA, 28 bp, Unknown
  Description of Unknown: HPRT sequence
acccagatacaaacaatggatagaaaac

SEQ ID NO: 659 - DNA, 28 bp, Unknown
  Description of Unknown: HPRT sequence
ctgggatgaactctgggcagaattcaca

SEQ ID NO: 660 - DNA, 28 bp, Unknown
  Description of Unknown: HPRT sequence
atgcagtctaagaatacagacagatcag

SEQ ID NO: 661 - DNA, 28 bp, Unknown
  Description of Unknown: HPRT sequence
tgcacaggggctgaagttgtcccacagg

SEQ ID NO: 662 - DNA, 28 bp, Unknown
  Description of Unknown: HPRT sequence
tggccaggaggctggttgcaaacatttt

SEQ ID NO: 663 - DNA, 28 bp, Unknown
  Description of Unknown: HPRT sequence
ttgaatgtgatttgaaaggtaatttagt

SEQ ID NO: 664 - DNA, 28 bp, Unknown
  Description of Unknown: HPRT sequence
aagctgatgatttaagctttggcggttt

SEQ ID NO: 665 - DNA, 28 bp, Unknown
  Description of Unknown: HPRT sequence
gtggggtaattgatccatgtatgccatt

SEQ ID NO: 666 - DNA, 28 bp, Unknown
  Description of Unknown: HPRT sequence
gggtggccaaaggaactgcgcgaacctc

SEQ ID NO: 667 - DNA, 28 bp, Unknown
  Description of Unknown: HPRT sequence
atcaactggagttggactgtaataccag

SEQ ID NO: 668 - DNA, 28 bp, Unknown
  Description of Unknown: HPRT sequence
ctttacagagacaagaggaataaaggaa

SEQ ID NO: 669 - DNA, 28 bp, Homo sapiens
cctatccattgcactatgctttatttaa

SEQ ID NO: 670 - DNA, 28 bp, Homo sapiens
tttgggatagttatgaattcaatcttca

SEQ ID NO: 671 - DNA, 28 bp, Homo sapiens
cctgtgctgttgatctcataaatagaac

SEQ ID NO: 672 - DNA, 28 bp, Homo sapiens
ttgtggtttttaaataaagcatagtgca

SEQ ID NO: 673 - DNA, 28 bp, Homo sapiens
accaagaagacagactaaaatgaaaata

SEQ ID NO: 674 - DNA, 28 bp, Homo sapiens
ctgttgatagacactaaaagagtattag

SEQ ID NO: 675 - DNA, 28 bp, Homo sapiens
tgacacagtacctggcaccatagttgta

SEQ ID NO: 676 - DNA, 28 bp, Homo sapiens
gtactaggggtatggggataaaccagac

SEQ ID NO: 677 - DNA, 28 bp, Homo sapiens
gcaaagattgctgactacggcattgctc

SEQ ID NO: 678 - DNA, 28 bp, Homo sapiens
tgatggcagcattgggatacagtgtgaa

SEQ ID NO: 679 - DNA, 28 bp, Homo sapiens
gcaaagattgctgactacagcattgctc

SEQ ID NO: 680 - DNA, 28 bp, Homo sapiens
ggggcgatgctggggacggggacattag

SEQ ID NO: 681 - DNA, 28 bp, Homo sapiens
acgctgcgccggcggaggcggggccgcg

SEQ ID NO: 682 - DNA, 28 bp, Homo sapiens
aaggcgccgtgggggctgccgggacggg

SEQ ID NO: 683 - DNA, 28 bp, Homo sapiens
agtccccggaggcctcgggccgactcgc

SEQ ID NO: 684 - DNA, 28 bp, Homo sapiens
gcgctcagcaggtggtgaccttgtggac

SEQ ID NO: 685 - DNA, 28 bp, Homo sapiens
atggtgggagagactgtgaggcggcagc

SEQ ID NO: 686 - DNA, 28 bp, Homo sapiens
atggcgctcagcaggtggtgaccttgtg

SEQ ID NO: 687 - DNA, 28 bp, Homo sapiens
tgggagagactgtgaggcggcagctggg

SEQ ID NO: 688 - DNA, 28 bp, Homo sapiens
gccaggtagtactgtgggtactcgaagg

SEQ ID NO: 689 - DNA, 28 bp, Homo sapiens
gagccatggcagttctccatgctggccg

SEQ ID NO: 690 - DNA, 28 bp, Homo sapiens
cagtgggttcttgccgcagcagatggtg

SEQ ID NO: 691 - DNA, 28 bp, Homo sapiens
gtgacgatgaggcctctgctaccgtgtc

SEQ ID NO: 692 - DNA, 28 bp, Homo sapiens
ggggagacagggcaaggctggcagagag

SEQ ID NO: 693 - DNA, 28 bp, Homo sapiens
atgtccaggctgctgcctcggtcccatt

SEQ ID NO: 694 - DNA, 28 bp, Unknown
  Description of Unknown: CFTR sequence
attagaagtgaagtctggaaataaaacc

SEQ ID NO: 695 - DNA, 44 bp, Unknown
  Description of Unknown: CFTR sequence
agtgattatgggagaactggatgttcacagtcagtccacacgtc

SEQ ID NO: 696 - DNA, 28 bp, Unknown
  Description of Unknown: CFTR sequence
catcataggaaacaccaaagatgatatt

SEQ ID NO: 697 - DNA, 28 bp, Unknown
  Description of Unknown: CFTR sequence
atatagatacagaagcgtcatcaaagca

SEQ ID NO: 698 - DNA, 28 bp, Unknown
  Description of Unknown: CFTR sequence
gctttgatgacgcttctgtatctatatt

SEQ ID NO: 699 - DNA, 28 bp, Unknown
  Description of Unknown: CFTR sequence
ccaactagaagaggtaagaaactatgtg

SEQ ID NO: 700 - DNA, 28 bp, Unknown
  Description of Unknown: CFTR sequence
cctatgatgaatatagatacagaagcgt

SEQ ID NO: 701 - DNA, 28 bp, Unknown
  Description of Unknown: CFTR sequence
acaccaatgatattttctttaatggtgc

SEQ ID NO: 702 - DNA, 28 bp, Unknown
  Description of Unknown: TRAC sequence
ctatggacttcaagagcaacagtgctgt

SEQ ID NO: 703 - DNA, 28 bp, Unknown
  Description of Unknown: TRAC sequence
ctcatgtctagcacagttttgtctgtga

SEQ ID NO: 704 - DNA, 28 bp, Unknown
  Description of Unknown: TRAC sequence
gtgctgtggcctggagcaacaaatctga

SEQ ID NO: 705 - DNA, 28 bp, Unknown
  Description of Unknown: TRAC sequence
ttgctcttgaagtccatagacctcatgt

SEQ ID NO: 706 - DNA, 28 bp, Unknown
  Description of Unknown: TRAC sequence
gctgtggcctggagcaacaaatctgact

SEQ ID NO: 707 - DNA, 28 bp, Unknown
  Description of Unknown: TRAC sequence
ctgttgctcttgaagtccatagacctca

SEQ ID NO: 708 - DNA, 28 bp, Unknown
  Description of Unknown: TRAC sequence
ctgtggcctggagcaacaaatctgactt

SEQ ID NO: 709 - DNA, 28 bp, Unknown
  Description of Unknown: TRAC sequence
ctgactttgcatgtgcaaacgccttcaa

SEQ ID NO: 710 - DNA, 28 bp, Unknown
  Description of Unknown: TRAC sequence
ttgttgctccaggccacagcactgttgc

SEQ ID NO: 711 - DNA, 28 bp, Unknown
  Description of Unknown: TRAC sequence
tgaaagtggccgggtttaatctgctcat

SEQ ID NO: 712 - DNA, 28 bp, Unknown
  Description of Unknown: TRAC sequence
aggaggattcggaacccaatcactgaca

SEQ ID NO: 713 - DNA, 28 bp, Unknown
  Description of Unknown: TRAC sequence
gaggaggattcggaacccaatcactgac

SEQ ID NO: 714 - DNA, 28 bp, Unknown
  Description of Unknown: TRBC sequence
ccgtagaactggacttgacagcggaagt

SEQ ID NO: 715 - DNA, 28 bp, Unknown
  Description of Unknown: TRBC sequence
tctcggagaatgacgagtggacccagga

SEQ ID NO: 716 - DNA, 28 bp, Homo sapiens
ccagggcgcctgtgggatctgcatgcct

SEQ ID NO: 717 - DNA, 28 bp, Homo sapiens
cagtcgtctgggcggtgctacaactggg

SEQ ID NO: 718 - DNA, 28 bp, Homo sapiens
gaacacaggcacggctgaggggtcctcc

SEQ ID NO: 719 - DNA, 28 bp, Homo sapiens
ctgtggactatggggagctggatttcca

SEQ ID NO: 720 - DNA, 19 bp, Homo sapiens
cagtcgtctgggcggtgct

SEQ ID NO: 721 - DNA, 28 bp, Homo sapiens
acagtgcttcggcaggctgacagccagg

SEQ ID NO: 722 - DNA, 28 bp, Homo sapiens
acccggacctcagtggctttgcctggag

SEQ ID NO: 723 - DNA, 28 bp, Homo sapiens
actacctgggcataggcaacggaaccca

SEQ ID NO: 724 - DNA, 28 bp, Homo sapiens
tggcggtgggtacatgagctccaccttg

SEQ ID NO: 725 - DNA, 28 bp, Homo sapiens
gtatggctgcgacgtggggtcggacggg

SEQ ID NO: 726 - DNA, 28 bp, Homo sapiens
ttatctggatggtgtgagaacctggccc

SEQ ID NO: 727 - DNA, 28 bp, Homo sapiens
tcctctggacggtgtgagaacctggccc

SEQ ID NO: 728 - DNA, 28 bp, Homo sapiens
atggagccgcgggcgccgtggatagagc

SEQ ID NO: 729 - DNA, 28 bp, Homo sapiens
ctggctcgcggcgtcgctgtcgaaccgc

SEQ ID NO: 730 - DNA, 28 bp, Homo sapiens
tccaggagctcaggtcctcgttcagggc

SEQ ID NO: 731 - DNA, 28 bp, Homo sapiens
cggcggacaccgcggctcagatcaccca

SEQ ID NO: 732 - DNA, 28 bp, Homo sapiens
aggtggatgcccaggacgagctttgagg

SEQ ID NO: 733 - DNA, 28 bp, Homo sapiens
agggagcagaagcagcgcagcagcgcca

SEQ ID NO: 734 - DNA, 28 bp, Homo sapiens
ctggaggtggatgcccaggacgagcttt

SEQ ID NO: 735 - DNA, 28 bp, Homo sapiens
gagcagaagcagcgcagcagcgccacct

SEQ ID NO: 736 - DNA, 28 bp, Homo sapiens
cctcagtttcatggggattcaagggaac

SEQ ID NO: 737 - DNA, 28 bp, Homo sapiens
cctaggaggtcatgggcatttgccatgc

SEQ ID NO: 738 - DNA, 28 bp, Homo sapiens
tcgcggcgtcgctgtcgaaccgcacgaa

SEQ ID NO: 739 - DNA, 28 bp, Homo sapiens
ccaagaggggagccgcgggagccgtggg

SEQ ID NO: 740 - DNA, 28 bp, Homo sapiens
gaaataaggcatactggtattactaatg

SEQ ID NO: 741 - DNA, 28 bp, Homo sapiens
gaggagagcaggccgattacctgaccca

SEQ ID NO: 742 - DNA, 28 bp, Unknown
  Description of Unknown: DRA sequence
tctcccagggtggttcagtggcagaatt

SEQ ID NO: 743 - DNA, 28 bp, Unknown
  Description of Unknown: DRA sequence
gcgggggaaagagaggaggagagaagga

SEQ ID NO: 744 - DNA, 28 bp, Unknown
  Description of Unknown: TAP1 sequence
agaaggctgtgggctcctcagagaaaat

SEQ ID NO: 745 - DNA, 28 bp, Unknown
  Description of Unknown: TAP1 sequence
actctggggtagatggagagcagtacct

SEQ ID NO: 746 - DNA, 28 bp, Unknown
  Description of Unknown: TAP2 sequence
ttgcggatccgggagcagcttttctcct

SEQ ID NO: 747 - DNA, 28 bp, Unknown
  Description of Unknown: TAP2 sequence
ttgattcgagacatggtgtaggtgaagc

SEQ ID NO: 748 - DNA, 28 bp, Unknown
  Description of Unknown: Tapasin sequence
ccacagccagagcctcagcaggagcctg

SEQ ID NO: 749 - DNA, 28 bp, Unknown
  Description of Unknown: Tapasin sequence
cgcaagaggctggagaggctgaggactg

SEQ ID NO: 750 - DNA, 28 bp, Unknown
  Description of Unknown: Tapasin sequence
ctggatggggcttggctgatggtcagca

SEQ ID NO: 751 - DNA, 28 bp, Unknown
  Description of Unknown: Tapasin sequence
gcccgcgggcagttctgcgcgggggtca

SEQ ID NO: 752 - DNA, 28 bp, Unknown
  Description of Unknown: CIITA sequence
gctcccaggcagcgggcgggaggctgga

SEQ ID NO: 753 - DNA, 28 bp, Unknown
  Description of Unknown: CIITA sequence
ctactcgggccatcggcggctgcctcgg

SEQ ID NO: 754 - DNA, 28 bp, Unknown
  Description of Unknown: RFX5 sequence
ttgatgtcagggaagatctctctgatga

SEQ ID NO: 755 - DNA, 28 bp, Unknown
  Description of Unknown: RFX5 sequence
gctcgaaggcttggtggccggggccagt

SEQ ID NO: 756 - DNA, 23 bp, Artificial Sequence
  Description of Artificial Sequence: Synthetic oligonucleotide
gtctgccgttactgccctgtggg

SEQ ID NO: 757 - DNA, 23 bp, Artificial Sequence
  Description of Artificial Sequence: Synthetic oligonucleotide
gtaacggcagacttcacctcagg

SEQ ID NO: 758 - DNA, 23 bp, Artificial Sequence
  Description of Artificial Sequence: Synthetic oligonucleotide
gcaatatgaatcccatggagagg

SEQ ID NO: 759 - DNA, 23 bp, Artificial Sequence
  Description of Artificial Sequence: Synthetic oligonucleotide
gcatattctgcactcatcccagg

SEQ ID NO: 760 - DNA, 23 bp, Artificial Sequence
  Description of Artificial Sequence: Synthetic oligonucleotide
gggccccttcccggacacacagg

SEQ ID NO: 761 - DNA, 23 bp, Artificial Sequence
  Description of Artificial Sequence: Synthetic oligonucleotide
gcaggtctggggcgcgccaccgg

SEQ ID NO: 762 - DNA, 23 bp, Artificial Sequence
  Description of Artificial Sequence: Synthetic oligonucleotide
ggcccccgagcccaaggcgctgg

SEQ ID NO: 763 - DNA, 23 bp, Artificial Sequence
  Description of Artificial Sequence: Synthetic oligonucleotide
gcgctgcaaccggtgtacccggg

SEQ ID NO: 764 - DNA, 23 bp, Artificial Sequence
  Description of Artificial Sequence: Synthetic oligonucleotide
gcattgagatagtgtggggaagg

SEQ ID NO: 765 - DNA, 23 bp, Artificial Sequence
  Description of Artificial Sequence: Synthetic oligonucleotide
gctattggtcaaggcaaggctgg

SEQ ID NO: 766 - DNA, 23 bp, Artificial Sequence
  Description of Artificial Sequence: Synthetic oligonucleotide
gtgttcatctttggttttgtggg

SEQ ID NO: 767 - DNA, 23 bp, Artificial Sequence
  Description of Artificial Sequence: Synthetic oligonucleotide
ggtcctgccgctgcttgtcatgg

SEQ ID NO: 768 - DNA, 23 bp, Artificial Sequence
  Description of Artificial Sequence: Synthetic oligonucleotide
gcttctaccccaatgacttgtgg

SEQ ID NO: 769 - DNA, 23 bp, Artificial Sequence
  Description of Artificial Sequence: Synthetic oligonucleotide
gcctctgactgttggtggcgtgg

SEQ ID NO: 770 - DNA, 23 bp, Artificial Sequence
  Description of Artificial Sequence: Synthetic oligonucleotide
gccgtggcaaactggtactttgg

SEQ ID NO: 771 - DNA, 23 bp, Artificial Sequence
  Description of Artificial Sequence: Synthetic oligonucleotide
ggggccactagggacaggattgg

SEQ ID NO: 772 - DNA, 23 bp, Artificial Sequence
  Description of Artificial Sequence: Synthetic oligonucleotide
gtcaccaatcctgtccctagtgg

SEQ ID NO: 773 - DNA, 23 bp, Artificial Sequence
  Description of Artificial Sequence: Synthetic oligonucleotide
gtggccccactgtggggtggagg

SEQ ID NO: 774 - DNA, 23 bp, Artificial Sequence
  Description of Artificial Sequence: Synthetic oligonucleotide
gtcggcatgacgggaccggtcgg

SEQ ID NO: 775 - DNA, 23 bp, Artificial Sequence
  Description of Artificial Sequence: Synthetic oligonucleotide
gatgtgatgaaggagatgggagg

SEQ ID NO: 776 - DNA, 23 bp, Artificial Sequence
  Description of Artificial Sequence: Synthetic oligonucleotide
gtgctttgatgtaatccagcagg

SEQ ID NO: 777 - DNA, 23 bp, Artificial Sequence
  Description of Artificial Sequence: Synthetic oligonucleotide
gtcgccataacggagccggccgg

SEQ ID NO: 778 - DNA, 23 bp, Artificial Sequence
  Description of Artificial Sequence: Synthetic oligonucleotide
gtattgcaaaaggtaggaaaagg

SEQ ID NO: 779 - DNA, 23 bp, Artificial Sequence
  Description of Artificial Sequence: Synthetic oligonucleotide
gcatatctgggatgaactctggg

SEQ ID NO: 780 - DNA, 23 bp, Artificial Sequence
  Description of Artificial Sequence: Synthetic oligonucleotide
gcctcctggccatgtgcacaggg

SEQ ID NO: 781 - DNA, 23 bp, Artificial Sequence
  Description of Artificial Sequence: Synthetic oligonucleotide
gaagctgatgatttaagctttgg

SEQ ID NO: 782 - DNA, 23 bp, Artificial Sequence
  Description of Artificial Sequence: Synthetic oligonucleotide
gatcaattaccccacctgggtgg

SEQ ID NO: 783 - DNA, 23 bp, Artificial Sequence
  Description of Artificial Sequence: Synthetic oligonucleotide
gatgtctttacagagacaagagg

SEQ ID NO: 784 - DNA, 23 bp, Artificial Sequence
  Description of Artificial Sequence: Synthetic oligonucleotide
gatcaacagcacaggttttgtgg

SEQ ID NO: 785 - DNA, 23 bp, Artificial Sequence
  Description of Artificial Sequence: Synthetic oligonucleotide
gtcagggtactaggggtatgggg

SEQ ID NO: 786 - DNA, 23 bp, Artificial Sequence
  Description of Artificial Sequence: Synthetic oligonucleotide
gtcagcaatctttgcaatgatgg

SEQ ID NO: 787 - DNA, 23 bp, Artificial Sequence
  Description of Artificial Sequence: Synthetic oligonucleotide
gtctgggacgcaaggcgccgtgg

SEQ ID NO: 788 - DNA, 23 bp, Artificial Sequence
  Description of Artificial Sequence: Synthetic oligonucleotide
ggaggcctcgggccgactcgcgg

SEQ ID NO: 789 - DNA, 23 bp, Artificial Sequence
  Description of Artificial Sequence: Synthetic oligonucleotide
gccggtgatatgggcttcctggg

SEQ ID NO: 790 - DNA, 23 bp, Artificial Sequence
  Description of Artificial Sequence: Synthetic oligonucleotide
gagactgtgaggcggcagctggg

SEQ ID NO: 791 - DNA, 23 bp, Artificial Sequence
  Description of Artificial Sequence: Synthetic oligonucleotide
ggctcagccaggtagtactgtgg

SEQ ID NO: 792 - DNA, 23 bp, Artificial Sequence
  Description of Artificial Sequence: Synthetic oligonucleotide
gaacccactgggtgacgatgagg

SEQ ID NO: 793 - DNA, 23 bp, Artificial Sequence
  Description of Artificial Sequence: Synthetic oligonucleotide
gccctgtctcccccatgtccagg

SEQ ID NO: 794 - DNA, 23 bp, Artificial Sequence
  Description of Artificial Sequence: Synthetic oligonucleotide
gggagaactggagccttcagagg

SEQ ID NO: 795 - DNA, 23 bp, Artificial Sequence
  Description of Artificial Sequence: Synthetic oligonucleotide
gagggtaaaattaagcacagtgg

SEQ ID NO: 796 - DNA, 23 bp, Artificial Sequence
  Description of Artificial Sequence: Synthetic oligonucleotide
gagaatcaaaatcggtgaatagg

SEQ ID NO: 797 - DNA, 23 bp, Artificial Sequence
  Description of Artificial Sequence: Synthetic oligonucleotide
gacaccttcttccccagcccagg

SEQ ID NO: 798 - DNA, 23 bp, Artificial Sequence
  Description of Artificial Sequence: Synthetic oligonucleotide
gattaaacccggccactttcagg

SEQ ID NO: 799 - DNA, 23 bp, Artificial Sequence
  Description of Artificial Sequence: Synthetic oligonucleotide
gctgtcaagtccagttctacggg

SEQ ID NO: 800 - DNA, 23 bp, Artificial Sequence
  Description of Artificial Sequence: Synthetic oligonucleotide
ggcgccctggccagtcgtctggg

SEQ ID NO: 801 - DNA, 23 bp, Artificial Sequence
  Description of Artificial Sequence: Synthetic oligonucleotide
gtccacagagaacacaggcacgg

SEQ ID NO: 802 - DNA, 23 bp, Artificial Sequence
  Description of Artificial Sequence: Synthetic oligonucleotide
gcttcggcaggctgacagccagg

SEQ ID NO: 803 - DNA, 23 bp, Artificial Sequence
  Description of Artificial Sequence: Synthetic oligonucleotide
gtacccaccgccatactacctgg

SEQ ID NO: 804 - DNA, 23 bp, Artificial Sequence
  Description of Artificial Sequence: Synthetic oligonucleotide
gctgcgacgtggggtcggacggg

SEQ ID NO: 805 - DNA, 23 bp, Artificial Sequence
  Description of Artificial Sequence: Synthetic oligonucleotide
gcagccatacattatctggatgg

SEQ ID NO: 806 - DNA, 23 bp, Artificial Sequence
  Description of Artificial Sequence: Synthetic oligonucleotide
gcagccatacatcctctggacgg

SEQ ID NO: 807 - DNA, 23 bp, Artificial Sequence
  Description of Artificial Sequence: Synthetic oligonucleotide
gtggatagagcaggaggggccgg

SEQ ID NO: 808 - DNA, 23 bp, Artificial Sequence
  Description of Artificial Sequence: Synthetic oligonucleotide
gagccagaggatggagccgcggg

SEQ ID NO: 809 - DNA, 23 bp, Artificial Sequence
  Description of Artificial Sequence: Synthetic oligonucleotide
ggacctgagctcctggaccgcgg

SEQ ID NO: 810 - DNA, 23 bp, Artificial Sequence
  Description of Artificial Sequence: Synthetic oligonucleotide
gatgcccaggacgagctttgagg

SEQ ID NO: 811 - DNA, 23 bp, Artificial Sequence
  Description of Artificial Sequence: Synthetic oligonucleotide
gcgctgcttctgctccctggagg

SEQ ID NO: 812 - DNA, 23 bp, Artificial Sequence
  Description of Artificial Sequence: Synthetic oligonucleotide
ggggattcaagggaacaccctgg

SEQ ID NO: 813 - DNA, 23 bp, Artificial Sequence
  Description of Artificial Sequence: Synthetic oligonucleotide
gcaaatgcccatgacctcctagg

SEQ ID NO: 814 - DNA, 23 bp, Artificial Sequence
  Description of Artificial Sequence: Synthetic oligonucleotide
ggcgcccgcggctcccctcttgg

SEQ ID NO: 815 - DNA, 23 bp, Artificial Sequence
  Description of Artificial Sequence: Synthetic oligonucleotide
gttcacatctcccccgggcctgg

SEQ ID NO: 816 - DNA, 23 bp, Artificial Sequence
  Description of Artificial Sequence: Synthetic oligonucleotide
ggagaatgcgggggaaagagagg

SEQ ID NO: 817 - DNA, 23 bp, Artificial Sequence
  Description of Artificial Sequence: Synthetic oligonucleotide
gcccacagccttctgtactctgg

SEQ ID NO: 818 - DNA, 23 bp, Artificial Sequence
  Description of Artificial Sequence: Synthetic oligonucleotide
gttgattcgagacatggtgtagg

SEQ ID NO: 819 - DNA, 23 bp, Artificial Sequence
  Description of Artificial Sequence: Synthetic oligonucleotide
gctctggctgtggtcgcaagagg

SEQ ID NO: 820 - DNA, 23 bp, Artificial Sequence
  Description of Artificial Sequence: Synthetic oligonucleotide
gcagaactgcccgcgggccctgg

SEQ ID NO: 821 - DNA, 23 bp, Artificial Sequence
  Description of Artificial Sequence: Synthetic oligonucleotide
gctgcctgggagccctactcggg

SEQ ID NO: 822 - DNA, 23 bp, Artificial Sequence
  Description of Artificial Sequence: Synthetic oligonucleotide
gccttcgagctttgatgtcaggg

SEQ ID NO: 823 - DNA, 573 bp, Mus musculus
gtaagagttttatgttttttcatctctgcttgtatttttctagtaatggaagcctggtattttaaaatagttaaattttcctttagtgctgatttctagattattattactgttgttgttgttattattgtcattatttgcatctgagaacccttaggtggttatattattgatatatttttggtatctttgatgacaataatgggggattttgaaagcttagctttaaatttcttttaattaaaaaaaaatgctaggcagaatgactcaaattacgttggatacagttgaatttattacggtctcatagggcctgcctgctcgaccatgctatactaaaaattaaaagtgtgtgttactaattttataaatggagtttccatttatatttacctttatttcttatttaccattgtcttagtagatatttacaaacatgacagaaacactaaatcttgagtttgaatgcacagatataaacacttaacgggttttaaaaataataatgttggtgaaaaaatataactttgagtgtagcagagaggaaccattgccaccttcagattttcctgt

SEQ ID NO: 824 - DNA, 1993 bp, Mus musculus
acgatcgggaactggcatcttcagggagtagcttaggtcagtgaagagaagaacaaaaagcagcatattacagttagttgtcttcatcaatctttaaatatgttgtgtggtttttctctccctgtttccacagacaagagtgagatcgcccatcggtataatgatttgggagaacaacatttcaaaggcctgtaagttataatgctgaaagcccacttaatatttctggtagtattagttaaagttttaaaacacctttttccaccttgagtgtgagaattgtagagcagtgctgtccagtagaaatgtgtgcattgacagaaagactgtggatctgtgctgagcaatgtggcagccagagatcacaaggctatcaagcactttgcacatggcaagtgtaactgagaagcacacattcaaataatagttaattttaattgaatgtatctagccatgtgtggctagtagctcctttcctggagagagaatctggagcccacatctaacttgttaagtctggaatcttattttttatttctggaaaggtctatgaactatagttttgggggcagctcacttactaacttttaatgcaataagatctcatggtatcttgagaacattattttgtctctttgtagtactgaaaccttatacatgtgaagtaaggggtctatacttaagtcacatctccaaccttagtaatgttttaatgtagtaaaaaaatgagtaattaatttatttttagaaggtcaatagtatcatgtattccaaataacagaggtatatggttagaaaagaaacaattcaaaggacttatataatatctagccttgacaatgaataaatttagagagtagtttgcctgtttgcctcatgttcataaatctattgacacatatgtgcatctgcacttcagcatggtagaagtccatattcctttgcttggaaaggcaggtgttcccattacgcctcagagaatagctgacgggaagaggctttctagatagttgtatgaaagatatacaaaatctcgcaggtatacacaggcatgatttgctggttgggagagccacttgcctcatactgaggtttttgtgtctgcttttcagagtcctgattgccttttcccagtatctccagaaatgctcatacgatgagcatgccaaattagtgcaggaagtaacagactttgcaaagacgtgtgttgccgatgagtctgccgccaactgtgacaaatcccttgtgagtaccttctgattttgtggatctactttcctgctttctggaactctgtttcaaagccaatcatgactccatcacttaaggccccgggaacactgtggcagagggcagcagagagattgataaagccagggtgatgggaattttctgtgggactccatttcatagtaattgcagaagctacaatacactcaaaaagtctcaccacatgactgcccaaatgggagcttgacagtgacagtgacagtagatatgccaaagtggatgagggaaagaccacaagagctaaaccctgtaaaaagaactgtaggcaactaaggaatgcagagagaagaagttgccttggaagagcataccaactgcctctccaataccaatggtcatccctaaaacatacgtatgaataacatgcagactaagcaggctacatttaggaatatacatgtatttacataaatgtatatgcatgtaacaacaatgaatgaaaactgaggtcatggatctgaaagagagcaagggggcttacatgagagggtttggagggaggggttggagggagggaggtattattctttagttttacagggaacgtagtaaaaacataggcttctcccaaaggagcagagcccatgaggagctgtgcaaggttccccagcttgattttacctgctcctcaaattcccttgatttgtttttattataatgactttactcctagcttttagtgtcagatagaaaacatggaagg

SEQ ID NO: 825 - DNA, 1301 bp, Unknown
  Description of Unknown: promoter-less Factor IX coding sequence
tgacagtgtttttagaccatgaaaatgccaacaagattctcaacagacccaagaggtacaacagtggcaagctggaggaatttgtgcagggcaacctggaaagagaatgcatggaggagaagtgctcatttgaagaggccagggaggtctttgagaacacagagaggaccacagagttctggaagcagtatgtggatggggaccagtgtgagagcaacccctgccttaatgggggcagctgtaaagatgatattaatagctatgaatgctggtgcccctttggatttgaggggaaaaactgtgaattggatgttacttgcaacatcaaaaatggtagatgtgagcagttctgcaagaactctgcagacaataaagtggtctgctcctgcactgaagggtacagactggcagaaaaccagaagagttgtgagccagctgtgcccttcccctgtggcagagtttctgtgagccagaccagcaaactcaccagagctgaggctgtctttccagatgtggactatgtgaactccacagaagctgagactatcctggacaacattactcagagcacccagtccttcaatgacttcacaagggtggttggaggagaagatgccaagccagggcagtttccctggcaggtggtactgaatggaaaagttgatgctttctgtggagggagcattgtgaatgaaaaatggattgtcactgctgcccactgtgtggaaactggggtgaagatcactgtggtggctggggagcataatattgaagaaacagagcacactgaacagaaaagaaatgtgatcaggatcatcccccaccacaactacaatgcagccatcaacaaatacaaccatgacattgccctgctggagctggatgagcccctggtgctgaacagctatgtgacccccatctgtattgctgacaaggagtacacaaatatcttcctgaagtttggctctggctatgtgagtggctggggcagagtgttccacaagggaagatctgccctggtgctgcagtacctgagggtgccactggtggacagggccacctgcctgaggagcacaaagttcaccatttataacaacatgttttgtgctggcttccatgagggaggcagggacagctgccagggagattctggagggccccatgtgactgaggtggagggcacctcctttctgacaggcattatcagctggggagaggagtgtgccatgaagggcaagtatggcatctacaccaaggtgtccagatatgtcaactggatcaaggaaaagaccaaactgacc

SEQ ID NO: 826 - DNA, 1350 bp, Homo sapiens
taggaggctgaggcaggaggatcgcttgagcccaggagttcgagaccagcctgggcaacatagtgtgatcttgtatctataaaaataaacaaaattagcttggtgtggtggcgcctgtagtccccagccacttggaggggtgaggtgagaggattgcttgagcccgggatggtccaggctgcagtgagccatgatcgtgccactgcactccagcctgggcgacagagtgagaccctgtctcacaacaacaacaacaacaacaaaaaggctgagctgcaccatgcttgacccagtttcttaaaattgttgtcaaagcttcattcactccatggtgctatagagcacaagattttatttggtgagatggtgctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttatttt

SEQ ID NO: 827 - DNA, 1223 bp, Homo sapiens
tgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtgacacggactcaagtgggctggtgacccagtcagagttgtgcacatggcttagttttcatacacagcctgggctgggggtggggtgggagaggtcttttttaaaaggaagttactgttatagagggtctaagattcatccatttatttggcatctgtttaaagtagattagatcttttaagcccatcaattatagaaagccaaatcaaaatatgttgatgaaaaatagcaacctttttatctccccttcacatgcatcaagttattgacaaactctcccttcactccgaaagttccttatgtatatttaaaagaaagcctcagagaattgctgattcttgagtttagtgatctgaacagaaataccaaaattatttcagaaatgtacaactttttacctagtacaaggcaacatataggttgtaaatgtgtttaaaacaggtctttgtcttgctatggggagaaaagacatgaatatgattagtaaagaaatgacacttttcatgtgtgatttcccctccaaggtatggttaataagtttcactgacttagaaccaggcgagagacttgtggcctgggagagctggggaagcttcttaaatgagaaggaatttgagttggatcatctattgctggcaaagacagaagcctcactgcaagcactgcatgggcaagcttggctgtagaaggagacagagctggttgggaagacatggggaggaaggacaaggctagatcatgaagaaccttgacggcattgctccgtctaagtcatgagctgagcagggagatcctggttggtgttgcagaaggtttactctgtggccaaaggagggtcaggaaggatgagcatttagggcaaggagaccaccaacagccctcaggtcagggtgaggatggcctctgctaagctcaaggcgtgaggatgggaaggagggaggtattcgtaaggatgggaaggagggaggtattcgtgcagcatatgaggatgcagagtcagcagaactg

SEQ ID NO: 828 - DNA, 1515 bp, Artificial Sequence
  Description of Artificial Sequence: Synthetic polynucleotide
gaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaactagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtgggtagcgcttgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 829 - DNA, 4107 bp, Unknown
  Description of Unknown: Cas9 sequence
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 830 - DNA, 215 bp, Artificial Sequence
  Description of Artificial Sequence: Synthetic polynucleotide
gaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccac

SEQ ID NO: 831 - DNA, 1876 bp, Artificial Sequence; identical to SEQ ID NO: 13
  Description of Artificial Sequence: Synthetic polynucleotide

SEQ ID NO: 832 - DNA, 7116 bp, Artificial Sequence
  Description of Artificial Sequence: Synthetic polynucleotide
ttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggcctacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgcgtcgacctgcaggcagctgcgcgctcgctcgctcactgaggccgcccgggcgtcgggcgacctttggtcgcccggcctcagtgagcgagcgagcgcgcagagagggagtggccaactccatcactaggggttcctcctgcaggtgtagttaatgattaacccgccatgctacttatctacgtagccatgcggcgcgccgccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagacaacaccacggaattatcagtgcccagcaacctagcccctgtccagcagcgggcaaggcaggcggcgatgagttctgccgtggcgatcgggagggggaaagcgaaagtcccagaaaggagttgacaggtggtggcaatgccccagccagtgggggttgcgtcagcaaacacagagcacaccacgccacgttgacggacaacgggccacaactcctctaaaagagacagcaaccaggatttatacaaggaggagaaaacgaaagccgtacgggaagcaatagctagatacagaggctataaagcagcatatccacacagcgtaaaaggagcaacatagttaagaatatcagtcaatctttcacaaattttgtaatccagaggttgattaacaggaacagagcgtaaataacgggaaagtttcttaacatgtttgtcttgtggcaatacacctgaactagtaattacatatccctaaaaatgtaaatgattgccccaccattttgttttattaacatttaaatgtatacccaaatcaagaaaaacagaacaaatatgggaataaatggcggtaagatgctcttaattaattaggtcagtttggtcttttccttgatccagttgacatatctggacaccttggtgtagatgccatacttgcccttcatggcacactcctctccccagctgataatgcctgtcagaaaggaggtgccctccacctcagtcacatggggccctccagaatctccctggcagctgtccctgcctccctcatggaagccagcacaaaacatgttgttataaatggtgaactttgtgctcctcaggcaggtggccctgtccaccagtggcaccctcaggtactgcagcaccagggcagatcttcccttgtggaacactctgccccagccactcacatagccagagccaaacttcaggaagatatttgtgtactccttgtcagcaatacagatgggggtcacatagctgttcagcaccaggggctcatccagctccagcagggcaatgtcatggttgtatttgttgatggctgcattgtagttgtggtgggggatgatcctgatcacatttcttttctgttcagtgtgctctgtttcttcaatattatgctccccagccaccacagtgatcttcaccccagtttccacacagtgggcagcagtgacaatccatttttcattcacaatgctccctccacagaaagcatcaacttttccattcagtaccacctgccagggaaactgccctggcttggcatcttctcctccaaccacccttgtgaagtcattgaaggactgggtgctctgagtaatgttgtccaggatagtctcagcttctgtggagttcacatagtccacatctggaaagacagcctcagctctggtgagtttgctggtctggctcacagaaactctgccacaggggaagggcacagctggctcacaactcttctggttttctgccagtctgtacccttcagtgcaggagcagaccactttattgtctgcagagttcttgcagaactgctcacatctaccatttttgatgttgcaagtaacatccaattcacagtttttcccctcaaatccaaaggggcaccagcattcatagctattaatatcatctttacagctgcccccattaaggcaggggttgctctcacactggtccccatccacatactgcttccagaactctgtggtcctctctgtgttctcaaagacctccctggcctcttcaaatgagcacttctcctccatgcattctctttccaggttgccctgcacaaattcctccagcttgccactgttgtacctcttgggtctgttgagaatcttgttggcattttcatggtctaaaaacactgtcactgggcaagggaagaaaaaaaaggattgttaaatactgaagaagcggccgctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggccggccgcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctacttt

SEQ ID NO: 833 - DNA, 7817 bp, Artificial Sequence
  Description of Artificial Sequence: Synthetic polynucleotide
ttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggcctacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgcgtcgacctgcaggcagctgcgcgctcgctcgctcactgaggccgcccgggcgtcgggcgacctttggtcgcccggcctcagtgagcgagcgagcgcgcagagagggagtggccaactccatcactaggggttcctcctgcaggtgtagttaatgattaacccgccatgctacttatctacgtagccatgcggcgcgccgtctttctgtcaatgcacacatttctactggacagcactgctctacaattctcacactcaaggtggaaaaaggtgttttaaaactttaactaatactaccagaaatattaagtgggctttcagcattataacttacaggcctttgaaatgttgttctcccaaatcattataccgatgggcgatctcactcttgtctgtggaaacagggagagaaaaaccacacaacatatttaaagattgatgaagacaactaactgtaatatgctgctttttgttcttctcttcactgacctaagctactccctgaagatgccagttcccgatcggccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagacaacaccacggaattatcagtgcccagcaacctagcccctgtccagcagcgggcaaggcaggcggcgatgagttctgccgtggcgatcgggagggggaaagcgaaagtcccagaaaggagttgacaggtggtggcaatgccccagccagtgggggttgcgtcagcaaacacagagcacaccacgccacgttgacggacaacgggccacaactcctctaaaagagacagcaaccaggatttatacaaggaggagaaaacgaaagccgtacgggaagcaatagctagatacagaggctataaagcagcatatccacacagcgtaaaaggagcaacatagttaagaatatcagtcaatctttcacaaattttgtaatccagaggttgattaacaggaacagagcgtaaataacgggaaagtttcttaacatgtttgtcttgtggcaatacacctgaactagtaattacatatccctaaaaatgtaaatgattgccccaccattttgttttattaacatttaaatgtatacccaaatcaagaaaaacagaacaaatatgggaataaatggcggtaagatgctcttaattaattaggtcagtttggtcttttccttgatccagttgacatatctggacaccttggtgtagatgccatacttgcccttcatggcacactcctctccccagctgataatgcctgtcagaaaggaggtgccctccacctcagtcacatggggccctccagaatctccctggcagctgtccctgcctccctcatggaagccagcacaaaacatgttgttataaatggtgaactttgtgctcctcaggcaggtggccctgtccaccagtggcaccctcaggtactgcagcaccagggcagatcttcccttgtggaacactctgccccagccactcacatagccagagccaaacttcaggaagatatttgtgtactccttgtcagcaatacagatgggggtcacatagctgttcagcaccaggggctcatccagctccagcagggcaatgtcatggttgtatttgttgatggctgcattgtagttgtggtgggggatgatcctgatcacatttcttttctgttcagtgtgctctgtttcttcaatattatgctccccagccaccacagtgatcttcaccccagtttccacacagtgggcagcagtgacaatccatttttcattcacaatgctccctccacagaaagcatcaacttttccattcagtaccacctgccagggaaactgccctggcttggcatcttctcctccaaccacccttgtgaagtcattgaaggactgggtgctctgagtaatgttgtccaggatagtctcagcttctgtggagttcacatagtccacatctggaaagacagcctcagctctggtgagtttgctggtctggctcacagaaactctgccacaggggaagggcacagctggctcacaactcttctggttttctgccagtctgtacccttcagtgcaggagcagaccactttattgtctgcagagttcttgcagaactgctcacatctaccatttttgatgttgcaagtaacatccaattcacagtttttcccctcaaatccaaaggggcaccagcattcatagctattaatatcatctttacagctgcccccattaaggcaggggttgctctcacactggtccccatccacatactgcttccagaactctgtggtcctctctgtgttctcaaagacctccctggcctcttcaaatgagcacttctcctccatgcattctctttccaggttgccctgcacaaattcctccagcttgccactgttgtacctcttgggtctgttgagaatcttgttggcattttcatggtctaaaaacactgtcactgggcaagggaagaaaaaaaaggattgttaaatactgaagaaacaggaaaatctgaaggtggcaatggttcctctctgctacactcaaagttatattttttcaccaacattattatttttaaaacccgttaagtgtttatatctgtgcattcaaactcaagatttagtgtttctgtcatgtttgtaaatatctactaagacaatggtaaataagaaataaaggtaaatataaatggaaactccatttataaaattagtaacacacacttttaatttttagtatagcatggtcgagcaggcaggccctatgagaccgtaataaattcaactgtatccaacgtaatttgagtcattctgcctagcatttttttttaattaaaagaaatttaaagctaagctttcaaaatcccccattatgcggccgctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggccggccgcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctacttt

SEQ ID NO: 834 - DNA, 9661 bp, Artificial Sequence
  Description of Artificial Sequence: Synthetic polynucleotide
ttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggcctacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgcgtcgacctgcaggcagctgcgcgctcgctcgctcactgaggccgcccgggcgtcgggcgacctttggtcgcccggcctcagtgagcgagcgagcgcgcagagagggagtggccaactccatcactaggggttcctcctgcaggtgtagttaatgattaacccgccatgctacttatctacgtagccatgcggcgcgccccttccatgttttctatctgacactaaaagctaggagtaaagtcattataataaaaacaaatcaagggaatttgaggagcaggtaaaatcaagctggggaaccttgcacagctcctcatgggctctgctcctttgggagaagcctatgtttttactacgttccctgtaaaactaaagaataatacctccctccctccaacccctccctccaaaccctctcatgtaagcccccttgctctctttcagatccatgacctcagttttcattcattgttgttacatgcatatacatttatgtaaatacatgtatattcctaaatgtagcctgcttagtctgcatgttattcatacgtatgttttagggatgaccattggtattggagaggcagttggtatgctcttccaaggcaacttcttctctctgcattccttagttgcctacagttctttttacagggtttagctcttgtggtctttccctcatccactttggcatatctactgtcactgtcactgtcaagctcccatttgggcagtcatgtggtgagactttttgagtgtattgtagcttctgcaattactatgaaatggagtcccacagaaaattcccatcaccctggctttatcaatctctctgctgccctctgccacagtgttcccggggccttaagtgatggagtcatgattggctttgaaacagagttccagaaagcaggaaagtagatccacaaaatcagaaggtactcacaagggatttgtcacagttggcggcagactcatcggcaacacacgtctttgcaaagtctgttacttcctgcactaatttggcatgctcatcgtatgagcatttctggagatactgggaaaaggcaatcaggactctgaaaagcagacacaaaaacctcagtatgaggcaagtggctctcccaaccagcaaatcatgcctgtgtatacctgcgagattttgtatatctttcatacaactatctagaaagcctcttcccgtcagctattctctgaggcgtaatgggaacacctgcctttccaagcaaaggaatatggacttctaccatgctgaagtgcagatgcacatatgtgtcaatagatttatgaacatgaggcaaacaggcaaactactctctaaatttattcattgtcaaggctagatattatataagtcctttgaattgtttcttttctaaccatatacctctgttatttggaatacatgatactattgaccttctaaaaataaattaattactcatttttttactacattaaaacattactaaggttggagatgtgacttaagtatagaccccttacttcacatgtataaggtttcagtactacaaagagacaaaataatgttctcaagataccatgagatcttattgcattaaaagttagtaagtgagctgcccccaaaactatagttcatagacctttccagaaataaaaaataagattccagacttaacaagttagatgtgggctccagattctctctccaggaaaggagctactagccacacatggctagatacattcaattaaaattaactattatttgaatgtgtgcttctcagttacacttgccatgtgcaaagtgcttgatagccttgtgatctctggctgccacattgctcagcacagatccacagtctttctgtcaatgcacacatttctactggacagcactgctctacaattctcacactcaaggtggaaaaaggtgttttaaaactttaactaatactaccagaaatattaagtgggctttcagcattataacttacaggcctttgaaatgttgttctcccaaatcattataccgatgggcgatctcactcttgtctgtggaaacagggagagaaaaaccacacaacatatttaaagattgatgaagacaactaactgtaatatgctgctttttgttcttctcttcactgacctaagctactccctgaagatgccagttcccgatcgtgccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagacaacaccacggaattatcagtgcccagcaacctagcccctgtccagcagcgggcaaggcaggcggcgatgagttctgccgtggcgatcgggagggggaaagcgaaagtcccagaaaggagttgacaggtggtggcaatgccccagccagtgggggttgcgtcagcaaacacagagcacaccacgccacgttgacggacaacgggccacaactcctctaaaagagacagcaaccaggatttatacaaggaggagaaaacgaaagccgtacgggaagcaatagctagatacagaggctataaagcagcatatccacacagcgtaaaaggagcaacatagttaagaatatcagtcaatctttcacaaattttgtaatccagaggttgattaacaggaacagagcgtaaataacgggaaagtttcttaacatgtttgtgcaatacacctgaactagtaattacatatccctaaaaatgtaaatgattgccccaccattttgttttattaacccaaatcaagaaaaacagaacaaatatgggaataaatggcggtaagatgctcttaattaattaggtcagtttggtcttttccttgatccagttgacatatctggacaccttggtgtagatgcatacttgcccttcatggcacactcctctccccagctgataatgcctgtcagaaaggaggtgccctccacctcagtcacatggggccctccagaatctccctggcagctgtccctgcctccctcatggaagccagcacaaaacatgttgttataaatggtgaactttgtgctcctcaggcaggtggccctgtccaccagtggcaccctcaggtactgcagcaccagggcagatcttcccttgtggaacactctgccccagccactcacatagccagagccaaacttcaggaagatatttgtgtactccttgtcagcaatacagatgggggtcacatagctgttcagcaccaggggctcatccagctccagcagggcaatgtcatggttgtatttgttgatggctgcattgtagttgtggtgggggatgatcctgatcacatttcttttctgttcagtgtgctctgtttcttcaatattatgctccccagccaccacagtgatcttcaccccagtttccacacagtgggcagcagtgacaatccatttttcattcacaatgctccctccacagaaagcatcaacttttccattcagtaccacctgccagggaaactgccctggcttggcatcttctcctccaaccacccttgtgaagtcattgaaggactgggtgctctgagtaatgttgtccaggatagtctcagcttctgtggagttcacatagtccacatctggaaagacagcctcagctctggtgagtttgctggtctggctcacagaaactctgccacaggggaagggcacagctggctcacaactcttctggttttctgccagtctgtacccttcagtgcaggagcagaccactttattgtctgcagagttcttgcagaactgctcacatctaccatttttgatgttgcaagtaacatccaattcacagtttttcccctcaaatccaaaggggcaccagcattcatagctattaatatcatctttacagctgcccccattaaggcaggggttgctctcacactggtccccatccacatactgcttccagaactctgtggtcctctctgtgttctcaaagacctccctggcctcttcaaatgagcacttctcctccatgcattctctttccaggttgccctgcacaaattcctccagcttgccactgttgtacctcttgggtctgttgagaatcttgttggcattttcatggtctaaaaacactgtcactgggcaagggaagaaaaaaaaggattgttaaatactgaagaaacaggaaaatctgaaggtggcaatggttcctctctgctacactcaaagttatattttttcaccaacattattatttttaaaacccgttaagtgtttatatctgtgcattcaaactcaagatttagtgtttctgtcatgtttgtaaatatctactaagacaatggtaaataagaaataaaggtaaatataaatggaaactccatttataaaattagtaacacacacttttaatttttagtatagcatggtcgagcaggcaggccctatgagaccgtaataaattcaactgtatccaacgtaatttgagtcattctgcctagcatttttttttaattaaaagaaatttaaagctaagctttcaaaatcccccattattgtcatcaaagataccaaaaatatatcaataatataaccacctaagggttctcagatgcaaataatgacaataataacaacaacaacagtaataataatctagaaatcagcactaaaggaaaatttaactattttaaaataccaggcttccattactagaaaaatacaagcagagatgaaaaaacataaaactcttacgcggccgctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggccggccgcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctacttt

SEQ ID NO: 835 - DNA, 60 bp, Mus musculus
ggaaccattgccaccttcagattttcctgtacgatcgggaactggcatcttcagggagta

SEQ ID NO: 836 - DNA, 20 bp, Artificial Sequence
  Description of Artificial Sequence: Synthetic oligonucleotide
gatcgggaactggcatcttc

SEQ ID NO: 837 - DNA, 20 bp, Artificial Sequence
  Description of Artificial Sequence: Synthetic oligonucleotide
gatcgtacaggaaaatctga

SEQ ID NO: 838 - DNA, 20 bp, Artificial Sequence
  Description of Artificial Sequence: Synthetic oligonucleotide
atcgggaactggcatcttca

SEQ ID NO: 839 - DNA, 20 bp, Artificial Sequence
  Description of Artificial Sequence: Synthetic oligonucleotide
cgtacaggaaaatctgaagg

SEQ ID NO: 840 - DNA, 20 bp, Artificial Sequence
  Description of Artificial Sequence: Synthetic oligonucleotide
tcagattttcctgtacgatc

SEQ ID NO: 841 - DNA, 20 bp, Artificial Sequence
  Description of Artificial Sequence: Synthetic oligonucleotide
tttcctgtacgatcgggaac

(19) United States
(12) Patent Application Publication
(10) Pub. No.: US 2022/0290186 A1
(43) Pub. Date: Sep. 15, 2022

(54) GENE EDITING USING A MODIFIED CLOSED-ENDED DNA (CEDNA)

(71) Applicant: Generation Bio Co., Cambridge, MA (US)

(72) Inventors: Robert M. Kotin, Cambridge, MA (US); Douglas Kerr, Cambridge, MA (US); Phillip Samayoa, Cambridge, MA (US); Ozan Alkan, Cambridge, MA (US); Matthew J. Simmons, Cambridge, MA (US)

(21) Appl. No.: 16/769,671

(22) PCT Filed: Dec. 6, 2018
(86) PCT No.: PCT/US18/64242

§ 371 Date: Jun. 4, 2020

Related U.S. Application Data
(60) Provisional application No. 62/595,328, filed on Dec. 6, 2017.
(60) Provisional application No. 62/607,069, filed on Dec. 18, 2017.

(51) Int. Cl. C12N 15/90; A61K 48/00; C12N 15/11; C12N 15/64; C12N 15/86; C12N 15/66; C12N 9/22

(52) U.S. Cl. CPC C12N 15/90; A61K 48/0016; C12N 15/111; C12N 15/64; C12N 15/86; C12N 15/66; C12N 9/22; C12N 2310/20; C12N 2330/51; C12N 2710/14041; C12N 2750/14143

(57) ABSTRACT

The application describes ceDNA vectors having linear and continuous structure for gene editing. ceDNA vectors comprise an expression cassette flanked by two ITR sequences, where the expression cassette encodes a gene editing molecule. Some ceDNA vectors further comprise cis-regulatory elements, including regulatory switches. Further provided herein are methods and cell lines for reliable gene editing using the ceDNA vectors.

### CROSS REFERENCE TO RELATED APPLICATIONS

This application is a 35 U.S.C. § 371 national stage filing of International Application No. PCT/US2018/064242, filed on Dec. 6, 2018, which in turn claims benefit under 35 U.S.C. § 119(e) of U.S. Provisional Application No. 62/595,328 filed on Dec. 6, 2017 and U.S. Provisional Application No. 62/607,069, filed on Dec. 18, 2017. The contents of each of the aforementioned applications are incorporated herein by reference in their entireties.

### SEQUENCE LISTING

The instant application contains a Sequence Listing which has been submitted electronically in ASCII format and is hereby incorporated by reference in its entirety. Said ASCII copy, created on Dec. 6, 2018, is named 080170-090470WOPT_SL.txt and is 198,924 bytes in size.

### TECHNICAL FIELD

The present invention relates to the field of gene therapy, including isolated polynucleotides having gene editing function. The disclosure also relates to nucleic acid constructs, promoters, vectors, and host cells including the polynucleotides as well as methods of delivering exogenous DNA sequences to a target cell, tissue, organ or organism. For example, the present disclosure provides gene editing non-viral DNA vectors.

### BACKGROUND

Gene therapy aims to improve clinical outcomes for patients suffering from either genetic mutations or acquired diseases caused by an aberration in the gene expression profile. Gene therapy includes the treatment or prevention of medical conditions resulting from defective genes or abnormal regulation or expression, e.g. underexpression or overexpression, that can result in a disorder, disease, malignancy, etc. For example, a disease or disorder caused by a defective gene might be treated, prevented or ameliorated by delivery of a corrective genetic material to a patient resulting in the therapeutic expression of the genetic material within the patient. A disease or disorder caused by a defective gene might be treated, prevented or ameliorated by altering or silencing a defective gene, e.g., removing all or part of the defective gene and/or editing a specific part of the defective gene with a corrective genetic material to a patient resulting in the therapeutic expression of the genetic material within the patient.

The basis of gene therapy is to supply a transcription cassette with an active gene product (sometimes referred to as a transgene), e.g., that can result in a positive gain-of-function effect, a negative loss-of-function effect, or another outcome, such as, e.g., an oncolytic effect. Gene therapy can also be used to treat a disease or malignancy caused by other factors. Human monogenic disorders can be treated by the delivery and expression of a normal gene to the target cells. Delivery and expression of a corrective gene in the patient's target cells can be carried out via numerous methods, including the use of engineered viruses and viral gene delivery vectors. Among the many virus-derived vectors available (e.g., recombinant retrovirus, recombinant lentivirus, recombinant adenovirus, and the like), recombinant adeno-associated virus (rAAV) is gaining popularity as a versatile vector in gene therapy.

Adeno-associated viruses (AAV) belong to the parvoviridae family and more specifically constitute the dependoparvovirus genus. The AAV genome is composed of a linear single-stranded DNA molecule which contains approximately 4.7 kilobases (kb) and consists of two major open reading frames (ORFs) encoding the non-structural Rep (replication) and structural Cap (capsid) proteins. A second ORF within the cap gene was identified that encodes the assembly-activating protein (AAP). The DNAs flanking the AAV coding regions are two cis-acting inverted terminal repeat (ITR) sequences, approximately 145 nucleotides in length, with interrupted palindromic sequences that can be folded into energetically-stable hairpin structures that function as primers of DNA replication. In addition to their role in DNA replication, the ITR sequences have been shown to be involved in viral DNA integration into the cellular genome, rescue from the host genome or plasmid, and encapsidation of viral nucleic acid into mature virions (Muzyczka, (1992) Curr. Top. Micro. Immunol. 158:97-129).

Vectors derived from AAV (i.e., recombinant AAV (rAVV) or AAV vectors) are attractive for delivering genetic material because (i) they are able to infect (transduce) a wide variety of non-dividing and dividing cell types including myocytes and neurons; (ii) they are devoid of the virus structural genes, thereby diminishing the host cell responses to virus infection, e.g., interferon-mediated responses; (iii) wild-type viruses are considered non-pathologic in humans; (iv) in contrast to wild type AAV, which are capable of integrating into the host cell genome, replication-deficient AAV vectors lack the rep gene and generally persist as episomes, thus limiting the risk of insertional mutagenesis or genotoxicity; and (v) in comparison to other vector systems, AAV vectors are generally considered to be relatively poor immunogens and therefore do not trigger a significant immune response (see ii), thus gaining persistence of the vector DNA and potentially, long-term expression of the therapeutic transgenes. AAV vectors can also be produced and formulated at high titer and delivered via intra-arterial, intra-venous, or intra-peritoneal injections allowing vector distribution and gene transfer to significant muscle regions through a single injection in rodents (Goyenvalle et al., 2004; Fougerousse et al., 2007; Koppanati et al., 2010; Wang et al., 2009) and dogs. In a clinical study to treat spinal muscular dystrophy type 1, AAV vectors were delivered systemically with the intention of targeting the brain resulting in apparent clinical improvements.

However, there are several major deficiencies in using AAV particles as a gene delivery vector. One major drawback associated with rAAV is its limited viral packaging capacity of about 4.5 kb of heterologous DNA (Dong et al., 1996; Athanasopoulos et al., 2004; Lai et al., 2010). As a result, use of AAV vectors has been limited to less than 150,000 Da protein coding capacity. The second drawback is that as a result of the prevalence of wild-type AAV infection in the population, candidates for rAAV gene therapy have to be screened for the presence of neutralizing antibodies that eliminate the vector from the patient. A third drawback is related to the capsid immunogenicity that prevents re-administration to patients that were not excluded from an initial treatment. The immune system in the patient can respond to the vector which effectively acts as a “booster” shot to stimulate the immune system generating high titer anti-AAV antibodies that preclude future treatments. Some recent reports indicate concerns with immunogenicity in high dose situations. Another notable drawback is that the onset of AAV-mediated gene expression is relatively slow, given that single-stranded AAV DNA must be converted to double-stranded DNA prior to heterologous gene expression. While attempts have been made to circumvent this issue by constructing double-stranded DNA vectors, this strategy further limits the size of the transgene expression cassette that can be integrated into the AAV vector (McCarty, 2008; Varenika et al., 2009; Foust et al., 2009).

Additionally, conventional AAV virions with capsids are produced by introducing a plasmid or plasmids containing the AAV genome, rep genes, and cap genes (Grimm et al., 1998). Upon introduction of these helper plasmids in trans, the AAV genome is “rescued” (i.e., released and subsequently amplified) from the host genome, and is further encapsidated (viral capsids) to produce biologically active AAV vectors. However, such encapsidated AAV virus vectors were found to inefficiently transduce certain cell and tissue types. The capsids also induce an immune response.

Accordingly, use of adeno-associated virus (AAV) vectors for gene therapy is limited due to the single administration to patients (owing to the patient immune response), the limited range of transgene genetic material suitable for delivery in AAV vectors due to minimal viral packaging capacity (about 4.5 kb) of the associated AAV capsid, as well as the slow AAV-mediated gene expression. The applications for rAAV clinical gene therapies are further encumbered by patient-to-patient variability not predicted by dose response in syngeneic mouse models or in other model species.

Current gene editing approaches such as those utilizing AAV to deliver a donor template, are problematic and have several limitations. First, the size of the donor template and for example, the homology arms for inducing homology-directed repair (HDR) are constrained by the packaging requirements within the AAV particle. Second, immunogenicity induced by the AAV administration precludes re-dosing and therefore, the gene editing process can only be done once. Finally, baseline immunity against AAV precludes a substantial proportion of patients from receiving the potential gene editing therapy. The inventors have observed other limitations of current gene editing approaches relating to the various components such as nuclease(s), promoter(s) guide RNA(s) (if Cas9 is the nuclease), the ‘corrected gene’ donor template(s) (e.g., a homology-directed recombination (HDR) repair template) and the separate delivery of homology regions. The current delivery of components is also problematic as components cannot be packaged in a single delivery particle and the use of multiple particles can raise immunogenicity issues. Since gene editing requires all the components are present within a single cell which is to be edited, the efficiency of gene editing is low as many cells do not get all of the delivered components.

Recombinant capsid-free AAV vectors can be obtained as an isolated linear nucleic acid molecule comprising an expressible transgene and promoter regions flanked by two wild-type AAV inverted terminal repeat sequences (ITRs) including the Rep binding and terminal resolution sites. These recombinant AAV vectors are devoid of AAV capsid protein encoding sequences, and can be single-stranded, double-stranded or duplex with one or both ends covalently linked through the two wild-type ITR palindrome sequences (e.g., WO2012/123430, U.S. Pat. No. 9,598,703). They avoid many of the problems of AAV-mediated gene therapy in that the transgene capacity is much higher, transgene expression onset is rapid, and the patient immune system does recognize the DNA molecules as a virus to be cleared. However, constant expression of a transgene may not be desirable in all instances, and AAV canonical wild type ITRs may not be optimized for ceDNA function.

There is need in the field for a technology that allows precise targeting of nuclease activity (or other protein activities) to distinct locations within a target DNA in a manner that does not require the design of a new protein for each new target sequence. In addition, there is a need in the art for methods of controlling gene expression with minimal off-target effects, and there remains an important unmet need for controllable recombinant DNA vectors with improved production and/or expression properties.

### BRIEF DESCRIPTION OF THE INVENTION

The invention described herein is a non-viral capsid-free DNA vector with covalently-closed ends (referred to herein as a “closed-ended DNA vector” or a “ceDNA vector”) for gene editing. The ceDNA vectors described herein are cap sid-free, linear duplex DNA molecules formed from a continuous strand of complementary DNA with covalently-closed ends (linear, continuous and non-encapsidated structure), which comprise a 5′ inverted terminal repeat (ITR) sequence and a 3′ ITR sequence, where the 5′ ITR and the 3′ ITR can have the same symmetrical three-dimensional organization with respect to each other, (i.e., symmetrical or substantially symmetrical), or alternatively, the 5′ ITR and the 3′ ITR can have different three-dimensional organization with respect to each other (i.e., asymmetrical ITRs). In addition, the ITRs can be from the same or different serotypes. In some embodiments, a ceDNA vector for gene editing can comprise ITR sequences that have a symmetrical three-dimensional spatial organization such that their structure is the same shape in geometrical space, or have the same A, C-C′ and B-B′ loops in 3D space (i.e., they are the same or are mirror images with respect to each other). In such an embodiment, a symmetrical ITR pair, or substantially symmetrical ITR pair can both be modified ITRs (e.g., mod-ITRs) in the same manner and do not both have to be wild-type ITRs. A mod-ITR pair can have the same sequence which has one or more modifications from wild-type ITR and are reverse complements (inverted) of each other. In alternative embodiments, a modified ITR pair are substantially symmetrical as defined herein, that is, the modified ITR pair can have a different sequence but have corresponding or the same symmetrical three-dimensional shape. In some embodiments, one ITR can be from one AAV serotype, and the other ITR can be from a different AAV serotype.

Accordingly, some aspects of the technology described herein relate to a ceDNA vector for gene editing that comprise ITR sequences selected from any of: (i) at least one WT ITR and at least one modified AAV inverted terminal repeat (ITR) (e.g., asymmetric modified ITRs); (ii) two modified ITRs where the mod-ITR pair have a different three-dimensional spatial organization with respect to each other (e.g., asymmetric modified ITRs), or (iii) symmetrical or substantially symmetrical WT-WT ITR pair, where each WT-ITR has the same three-dimensional spatial organization, or (iv) symmetrical or substantially symmetrical modified ITR pair, where each mod-ITR has the same three-dimensional spatial organization. The ceDNA vectors disclosed herein can be produced in eukaryotic cells, thus devoid of prokaryotic DNA modifications and bacterial endotoxin contamination in insect cells.

More particularly, embodiments of the invention are based on methods and compositions comprising a gene editing ceDNA vector that can express a transgene which is a gene editing molecule in a host cell (e.g., a transgene is a nuclease such as ZFN, TALEN, Cas; one or more guide RNA; CRISPR; a ribonucleoprotein (RNP), or any combination thereof) and result in efficient genome editing. The ceDNA vectors described herein are not limited by size, thereby permitting, for example, expression of all of the components necessary for a gene editing system from a single vector (e.g., a CRISPR/Cas gene editing system (e.g., a Cas9 or modified Cas9 enzyme, a guide RNA and/or a homology directed repair template), or for a TALEN or Zinc Finger system). However, it is also contemplated that one or two of such components encoded on a single ceDNA vector, while the remaining component(s) can be expressed on a separate ceDNA vector or a traditional plasmid.

The technology described herein relates to a ceDNA vector containing two AAV inverted terminal repeat sequences (ITR) flanking a transgene or heterologous nucleic acid, where the heterologous nucleic acid is a gene editing nucleic acid sequence. In all aspects provided herein, the gene editing nucleic acid sequence encodes a gene editing molecule selected from the group consisting of: a sequence specific nuclease, one or more guide RNA, CRISPR/Cas, a ribonucleoprotein (RNP), or deactivated CAS for CRISPRi or CRISPRa systems, or any combination thereof.

In some embodiments, the ceDNA vector comprises: (1) an expression cassette comprising a cis-regulatory element, a promoter and at least one transgene (e.g., a gene editing molecule); or (2) a promoter operably linked to at least one transgene (e.g., a gene editing molecule), and (3) two self-complementary sequences, e.g., asymmetrical or symmetrical or substantially symmetrical ITRs as defined herein, flanking said expression cassette, wherein the ceDNA vector is not associated with a capsid protein. In some embodiments, the ceDNA vector comprises two self-complementary sequences found in an AAV genome, where at least one ITR comprises an operative Rep-binding element (RBE) (also sometimes referred to herein as “RBS”) and a terminal resolution site (trs) of AAV or a functional variant of the RBE, and one or more cis-regulatory elements operatively linked to a transgene. In some embodiments, the ceDNA vector comprises additional components to regulate expression of the transgene (e.g., a gene editing molecule), for example, regulatory switches, which are described herein in the section entitled “Regulatory Switches” for controlling and regulating the expression of the transgene, and can include a regulatory switch, e.g., a kill switch to enable controlled cell death of a cell comprising a ceDNA vector.

In some embodiments, a ceDNA vector for gene editing described herein can be used for knock-in of desired nucleic acid sequence. In particular, the methods and compositions described herein can be used to introduce a new nucleic acid sequence, correct a mutation of a genomic sequence or introduce a mutation into a target gene sequence in a host cell. Such methods can be referred to as “DNA knock-in systems.”

In some embodiments, a gene editing ceDNA vector disclosed herein comprises homology arms, e.g., at increase specificity of targeting to a target gene. Homology-directed repair (HDR) is a process of homologous recombination where a DNA template is used to provide the homology necessary for precise repair of a double-strand break (DSB) of insertion of the donor sequence of interest. For example, in one nonlimiting example, a ceDNA vector for gene editing can comprise a 5′ and 3′ homology arm to a specific gene, or target intergration site. In some embodiments, a specific restriction site may be engineered 5′ to the 5′ homology arm, 3′ to the 3′ homology arm, or both. When the ceDNA vector is cleaved with the one or more restriction endonucleases specific for the engineered restriction site(s), the resulting cassette comprises the 5′ homology arm-donor sequence-3′ homology arm, and can be more readily recombined with the desired genomic locus. In some embodiments, in the genomic DNA sequence to be targeted, located 5′ of, and near to where the 5′ end of the 3′ homology arm homologous, and/or located 3′ of, and near to where the 3′ end of the 5′ homology arm is homologous, there is a sgRNA target sequence (e.g., see FIGS. 17 and 18A). It will be appreciated by one of ordinary skill in the art that this cleaved cassette may additionally comprise other elements such as, but not limited to, one or more of the following: a regulatory region, a nuclease, and an additional donor sequence. In certain aspects, the ceDNA vector itself may encode the restriction endonuclease such that upon delivery of the ceDNA vector to the nucleus, the restriction endonuclease is expressed and able to cleave the vector. In certain aspects, the restriction endonuclease is encoded on a second ceDNA vector which is separately delivered. In certain aspects, the restriction endonuclease is introduced to the nucleus by a non-ceDNA-based means of delivery. In certain embodiments, the restriction endonuclease is introduced after the ceDNA vector is delivered to the nucleus. In certain embodiments, the restriction endonuclease and the ceDNA vector are transported to the nucleus simultaneously. In certain embodiments, the restriction endonuclease is already present upon introduction of the ceDNA vector.

Accordingly, in some embodiments, the technology described herein enables more than one gene editing ceDNA being delivered to a subject. As discussed herein, in one embodiment, a ceDNA can have the homology arms flanking a donor sequence that targets a specific target gene or locus, and can in some embodiments, also include one or more guide RNAs (e.g., sgRNA) for targeting the cutting of the genomic DNA, as described herein, and another ceDNA can comprise a nuclease enzyme and activator RNA, as described herein for the actual gene editing steps.

In another embodiment of this aspect and all other aspects provided herein, the sequence-specific nuclease comprises: a TAL-nuclease, a zinc-finger nuclease (ZFN), a meganuclease, a megaTAL, or an RNA guided endonuclease (e.g., CAS9, cpfl, dCAS9, nCAS9).

In another embodiment of this aspect and all other aspects provided herein, the gene editing nucleic acid sequence is a homology-directed repair template.

In another embodiment of this aspect and all other aspects provided herein, the homology-directed repair template comprises a 5′ homology arm, a donor sequence, and a 3′ homology arm.

In another embodiment of this aspect and all other aspects provided herein, the composition further comprises a nucleic acid sequence that encodes an endonuclease, wherein the endonuclease cleaves or nicks at a specific endonuclease site on DNA of a target gene or a target site on the ceDNA vector.

In another embodiment of this aspect and all other aspects provided herein, the 5′ homology arm is homologous to a nucleotide sequence upstream of the DNA endonuclease cutting or nicking site on a chromosome.

In another embodiment of this aspect and all other aspects provided herein, the 3′ homology arm is homologous to a nucleotide sequence downstream of the DNA endonuclease cutting or nicking site.

In another embodiment of this aspect and all other aspects provided herein, the homology arms are each about 250 to 2000 bp.

In another embodiment of this aspect and all other aspects provided herein, the DNA endonuclease comprises: a TAL-nuclease, a zinc-finger nuclease (ZFN), or an RNA guided endonuclease (e.g., Cas9 or Cpf1).

In another embodiment of this aspect and all other aspects provided herein, the RNA guided endonuclease comprises a Cas enzyme.

In another embodiment of this aspect and all other aspects provided herein, the Cas enzyme is Cas9.

In another embodiment of this aspect and all other aspects provided herein, the Cas enzyme is nicking Cas9 (nCas9).

In another embodiment of this aspect and all other aspects provided herein, the nCas9 comprises a mutation in the HNH or RuVc domain (e.g. D10A) of Cas.

In another embodiment of this aspect and all other aspects provided herein, the Cas enzyme is deactivated Cas nuclease (dCas) that complexes with a gRNA that targets a promoter region of a target gene.

In another embodiment of this aspect and all other aspects provided herein, the composition further comprises a KRAB effector domain.

In another embodiment of this aspect and all other aspects provided herein, the dCas is fused to a heterologous transcriptional activation domain that can be directed to a promoter region.

In another embodiment of this aspect and all other aspects provided herein, the dCas fusion is directed to a promoter region of a target gene by a guide RNA that recruits additional transactivation domains to upregulate expression of the target gene.

In another embodiment of this aspect and all other aspects provided herein, the dCas is S. pyogenes dCas9.

In another embodiment of this aspect and all other aspects provided herein, the guide RNA sequence targets the proximity of the promoter of a target gene and CRISPR silences the target gene (CRISPRi system). As used herein, the phrase “proximity of the promoter of a target gene” refers to a region that is physically on, adjacent or near the promoter sequence of the target gene and a catalytically inactive DNA endonuclease can function to inhibit expression of the target gene. In some embodiments, “proximity to the promoter” refers to a sequence within the promoter sequence itself, directly adjacent to the promoter sequence (either end) or 1, 2, 3, 4, 5, 6, 7, 8, 9, 10, 15, 20, 25, 30, 35, 40, 45, 50 nucleotides or more from a terminal end of the promoter sequence.

In another embodiment of this aspect and all other aspects provided herein, the guide RNA sequence targets the transcriptional start site of a target gene and activates, or modulates, the target gene (CRISPRa system). As used herein, the term “transcriptional start site of a target gene” refers to a region that is physically on, adjacent or near the transcriptional start sequence (“ATG”; initiating methionine) of the target gene and a catalytically inactive DNA endonuclease can function to recruit transcriptional machinery, such as RNA polymerase, to increase expression of the target gene, for example, by at least 10%. In some embodiments, the guide RNA may comprise a sequence that includes the “ATG” transcriptional start site. In other embodiments, the guide RNA may comprise a sequence directly upstream of the transcriptional start site. In additional embodiments, the guide RNA can comprise a sequence 1, 2, 3, 4, 5, 6, 7, 8, 9, 10, 15, 20, 25, 30, 35, 40, 45, 50 nucleotides or more upstream of the transcriptional start site, provided that the distance is not so large that the recruited translational machinery does not function to enhance expression of the target gene.

In another embodiment of this aspect and all other aspects provided herein, the guide RNA sequence targets the proximity of a promoter of a target gene and activates, or modulates, the target gene (CRISPRa system), for example, to increase expression of the target gene.

In another embodiment of this aspect and all other aspects provided herein, the composition further comprises a nucleic acid encoding at least one guide RNA (gRNA) for a RNA-guided DNA endonuclease.

In another embodiment of this aspect and all other aspects provided herein, the guide RNA (gRNA) targets a splice acceptor or splice donor site of a defective gene to effect non-homologous end joining (NHEJ) and correction of the defective gene for expression of functional protein. The term “splice acceptor” as used herein refers to a nucleic acid sequence at the 3′ end of an intron where it junctions with an exon. The consensus sequences for a splice acceptor include, but are not limited to: NTN(TC) (TC) (TC)TTT (TC) (TC)(TC) (TC) (TC) (TC)NCAGg (SEQ ID NO: 558). The intronic sequences are represented by upper case and the exonic sequence by lower case font. The term “splice donor” as used herein refers to a nucleic acid sequence at the 5′ end of an intron where it junctions with an exon. The consensus sequence for a splice donor sequence includes, but is not limited to: naggt(ag)aGT (SEQ ID NO: 559). The intronic sequences are represented by upper case and the exonic sequence by lower case font. Theses sequences represent those of which are conserved from viral to primate genomes.

In another embodiment of this aspect and all other aspects provided herein, the vector encodes multiple copies of one guide RNA sequence.

In another embodiment of this aspect and all other aspects provided herein, the composition further comprises a regulatory sequence operably linked to the nucleic acid sequence encoding the gene editing sequence.

In another embodiment of this aspect and all other aspects provided herein, the regulatory sequence comprises an enhancer and/or a promoter. In certain embodiments the promoter is an inducible promoter.

In another embodiment of this aspect and all other aspects provided herein, a promoter is operably linked to the nucleic acid sequence encoding the DNA endonuclease, wherein the nucleic acid sequence encoding the DNA endonuclease further comprises an intron sequence upstream of the endonuclease sequence, and wherein the intron comprises a nuclease cleavage site.

In another embodiment of this aspect and all other aspects provided herein, a poly-A-site is upstream and proximate to the 5′ homology arm.

In another embodiment of this aspect and all other aspects provided herein, the donor sequence is foreign to the 5′ homology arm or the 3′ homology arm.

In another embodiment of this aspect and all other aspects provided herein, the 5′ homology arm or the 3′ homology arm are proximal to the at least one ITR as defined herein.

In another embodiment of this aspect and all other aspects provided herein, the nucleotide sequence encoding a nuclease is cDNA.

In another embodiment, the editing is directed at RNA instead of DNA. For example, using Cas13, such as Cas13 from Prevotella spp. bacteria. This enzyme is combined with another molecule that corrects the RNA. For example, the ADAR2 protein changes individual RNA's from adenosine to inosines. See e.g., Science, Cox, D. B. T. et al. 25 Oct. 2017 “RNA editing with CRISPR-Cas13. RNA editing and/or tracking using ceDNA vector(s) encoding a gene editing system as described herein can be performed with methods known in the art, for example, Abudayyeh et al. Science 353:1-9 (2016); O'Connell et al. Nature 516:263-266 (2014); Nelles et al. Cell 165:488-496 (2016); the contents of each of which are incorporated by reference herein in their entirety.

Another aspect provided herein relates to a method for genome editing comprising: contacting a cell with a gene editing system, wherein one or more components of the gene editing system are delivered to the cell by contacting the cell with a composition comprising the ceDNA vector as disclosed herein, wherein the ceDNA nucleic acid vector composition comprises flanking inverted terminal repeat (ITR) sequences where the ITR sequences are asymmetrical, symmetrical or substantially symmetrical relative to each other as defined herein, and at least one gene editing nucleic acid sequence.

In another embodiment of this aspect and all other aspects provided herein, the gene editing system is selected from the group consisting of: a TALEN system, a zinc-finger endonuclease (ZFN) system, a CRISPR/Cas system, A CRISPRi system, a CRISPRa system, and a meganuclease system.

In another embodiment of this aspect and all other aspects provided herein, the at least one gene editing nucleic acid sequence encodes a gene editing molecule selected from the group consisting of: an RNA guided nuclease, a guide RNA, guide DNA, ZFN, TALEN, a Cas, CRISPR/Cas molecule or orthologue thereof, a ribonucleoprotein (RNP), or deactivated CAS for CRISPRi or CRISPRa systems.

In another embodiment of this aspect and all other aspects provided herein, a single ceDNA vector comprises all components of the gene editing system.

In another embodiment of this aspect and all other aspects provided herein, the Cas protein is codon optimized for expression in the eukaryotic cell.

Also provided herein, in another aspect is a method of genome editing comprising administering to a cell an effective amount of a ceDNA composition as described herein, under conditions suitable and for a time sufficient to edit a target gene.

In another embodiment of this aspect and all other aspects provided herein, the target gene is targeted using one or more guide RNA sequences and edited by homology directed repair (HDR) in the presence of a HDR donor template.

In another embodiment of this aspect and all other aspects provided herein, the target gene is targeted using one guide RNA sequence and the target gene is edited by non-homologous end joining (NHEJ). In one embodiment, the guide RNA targets a splice donor or acceptor to promote exon skipping and expression of functional protein, e.g. dystrophin protein.

In another embodiment of this aspect and all other aspects provided herein, the method is performed in vivo to correct a single nucleotide polymorphism (SNP), or deletion or insertion, associated with a disease.

In another embodiment of this aspect and all other aspects provided herein, a disease suitable for gene editing using the ceDNA vectors disclosed herein is discussed in the sections entitled “Exemplary diseases to be treated with a gene editing ceDNA” and “Additional diseases for gene editing” herein. Exemplary disease to be treated are, for example, but not limited to, Duchene Muscular Dystrophy (DMD gene), transthyretin amyloidosis (ATTR) (correct mutTTR gene), ornithine transcarbamylase deficiency (OTC deficiency), haemophilia, cystic fibrosis, sickle cell anemia, hereditary hemochromatosis, cancer, or hereditary blindness, and genes to be corrected, include but are not limited to; erythropoietin, angiostatin, endostatin, superoxide dismutase (SOD1), globin, leptin, catalase, tyrosine hydroxylase, a cytokine, cystic fibrosis transmembrane conductance regulator (CFTR), or a peptide growth factor, and the like.

In another embodiment of this aspect and all other aspects provided herein, at least 2 different Cas proteins are present in the ceDNA vector, wherein one of the Cas proteins is catalytically inactive (Cas-i), and wherein the guide RNA associated with the Cas-I targets the promoter of the target cell, and wherein the DNA coding for the Cas-I is under the control of an inducible promoter so that it can turn-off the expression of the target gene at a desired time. As used herein, the term “catalytically inactive” refers to a molecule (e.g., an enzyme or a kinase) with a catalytic site that has been altered from an active state to an inactive state, thereby hindering its activity. A molecule can be rendered catalytically inactive for example, from denaturation, inhibitory binding, mutations to the catalytic site, or secondary processing (e.g., phosphorylation or other post-translational modifications). For example, a catalytically inactive, or deactivated Cas9 (dCas9), does not possess endonuclease activity and can be generated, for example, by introducing point mutations in the two catalytic residues, D10A and H840A, of the gene encoding Cas9. In one embodiment, a catalytically inactive state of a molecule refers to a molecule with less than 0.1% catalytic activity compared to its catalytically active state and further encompasses a molecule having any activity discernable by standard laboratory methods.

Also provided herein, in another aspect, is a method for editing a single nucleotide base pair in a target gene of a cell, the method comprising contacting a cell with a CRISPR/Cas gene editing system, wherein one or more components of the CRISPR/Cas gene editing system are delivered to the cell by contacting the cell with a close-ended DNA (ceDNA) nucleic acid vector composition, wherein the ceDNA nucleic acid vector composition is a linear close-ended duplex DNA comprising flanking terminal repeat (TR) sequences and at least one gene editing nucleic acid sequence for targeting a target gene or a regulatory sequence for the target gene, wherein the Cas protein expressed from the vector is catalytically inactive and is fused to a base editing moiety, wherein the method is performed under conditions and for a time sufficient to modulate expression of the target gene.

In another embodiment of this aspect and all other aspects provided herein, the base editing moiety comprises a single-strand-specific cytidine deaminase, a uracil glycosylase inhibitor, or a tRNA adenosine deaminase.

In another embodiment of this aspect and all other aspects provided herein, the catalytically inactive Cas protein expressed from the vector is dCas9.

In another embodiment of this aspect and all other aspects provided herein, the cell contacted is a T cell, or a CD34⁺ cell.

In another embodiment of this aspect and all other aspects provided herein, the target gene encodes for a programmed death protein (PD1), cytotoxic T-lymphocyte-associated antigen 4 (CTLA4), or tumor necrosis factor-α (TNF-α).

In another embodiment of this aspect and all other aspects provided herein, further comprising administering the cells (e.g. T cells or CD34+ cells) produced by a method described herein to a subject in need thereof.

In another embodiment of this aspect and all other aspects provided herein, the subject in need thereof has a viral infection, bacterial infection, cancer, or autoimmune disease.

Another aspect provided herein relates to a method of modulating expression of two or more target genes in a cell comprising: introducing into the cell: (i) a composition comprising a ceDNA vector that comprises flanking ITR sequences, where the ITR sequences are asymmetrical, symmetrical or substantially symmetrical relative to each other as defined herein, and a nucleic acid sequence encoding at least two guide RNAs complementary to two or more target genes, wherein the vector is a linear close-ended duplex DNA, (ii) a second composition comprising a ceDNA vector that comprises flanking ITR sequences, where the ITR sequences are asymmetrical, symmetrical or substantially symmetrical relative to each other as defined herein, and a nucleic acid sequence encoding at least two catalytically inactive DNA endonucleases that each associate with a guide RNA and bind to the two or more target genes, wherein the vector is a linear close-ended duplex DNA, and (iii) a third composition comprising a ceDNA vector that comprises flanking ITR sequences, where the ITR sequences are asymmetrical, symmetrical or substantially symmetrical relative to each other as defined herein, and a nucleic acid sequence encoding at least two transcriptional regulator proteins or domains, wherein the vector is a linear close-ended duplex DNA, and wherein the at least two guide RNAs, the at least two catalytically inactive RNA-guided endonucleases and the at least two transcriptional regulator proteins or domains are expressed in the cell, wherein two or more co-localization complexes form between a guide RNA, a catalytically inactive RNA-guided endonuclease, a transcriptional regulator protein or domain and a target gene, and wherein the transcriptional regulator protein or domain regulates expression of the at least two target genes.

In one aspect, non-viral capsid-free DNA vectors with covalently-closed ends are preferably linear duplex molecules, and are obtainable from a vector polynucleotide that encodes a heterologous nucleic acid operatively positioned between two inverted terminal repeat sequences (ITRs) (e.g. AAV ITRs), wherein at least one of the ITRs comprises a terminal resolution site and a replication protein binding site (RPS) (sometimes referred to as a replicative protein binding site), e.g. a Rep binding site. The 5′ ITR and 3′ ITR can be symmetrical or substantially symmetrical relative to each other where the 5′ and 3′ ITR have the same three-dimensional spatial organization (i.e., a symmetrical mod-ITR pair or a symmetrical or substantially symmetrical WT-ITR pair), or asymmetrical relative to each other such that the 5′ ITR and the 3′ ITR have different three-dimensional organization with respect to each other (i.e., asymmetrical ITRs) with respect to each other (e.g., a WT-ITR and a mod-ITR or a mod-ITR pair that, as these terms are defined herein.

In some embodiments, the two self-complementary sequences can be ITR sequences from any known parvovirus, for example a dependovirus such as AAV (e.g., AAV1-AAV12). Any AAV serotype can be used, including but not limited to a modified AAV2 ITR sequence, that retains a Rep-binding site (RBS) such as 5′-GCGCGCTCGCTCGCTC-3′ (SEQ ID NO: 531) and a terminal resolution site (trs) in addition to a variable palindromic sequence allowing for hairpin secondary structure formation. In some embodiments, the ITR is a synthetic ITR sequence that retains a functional Rep-binding site (RBS) such as 5′-GCGCGCTCGCTCGCTC-3′ (SEQ ID NO: 531) and a terminal resolution site (TRS) in addition to a variable palindromic sequence allowing for hairpin secondary structure formation. In some examples, an ITR sequence retains the sequence of the RBS, trs and the structure and position of a Rep binding element forming the terminal loop portion of one of the ITR hairpin secondary structure from the corresponding sequence of the wild-type AAV2 ITR.

In some embodiments, a ceDNA vector comprising an asymmetric ITR pair can comprise a ITR with a modification in the ITR corresponding to any of the modifications in ITR sequences or ITR partial sequences shown in any one or more of Table 4A or 4B herein, or one or more of Tables 2, 3, 4, 5, 6, 7, 8, 9 and 10A-10B of PCT application PCT/US18/49996 which is incorporated herein in its entirety by reference. As an exemplary example, the present disclosure provides a closed-ended DNA vector for gene editing that comprises asymmetrical ITRs, the ceDNA vector comprising a promoter operably linked to a transgene, where the ceDNA is devoid of capsid proteins and is: (a) produced from a ceDNA-plasmid (e.g., see Examples 1-2 and/or FIGS. 1A-1B) that encodes a mutated right side AAV2 ITR having the same number of intramolecularly duplexed base pairs as SEQ ID NO:2 or a mutated left side AAV2 ITR having the same number of intramolecularly duplexed base pairs as SEQ ID NO:51 in its hairpin secondary configuration (preferably excluding deletion of any AAA or TTT terminal loop in this configuration compared to these reference sequences), and (b) is identified as ceDNA using the assay for the identification of ceDNA by agarose gel electrophoresis under native gel and denaturing conditions in Example 1. Examples of such 5′ and 3′ modified ITR sequences for ceDNA vector comprising asymmetric ITRs are provided in Tables 4A or 4B herein, or one or more of Tables 2, 3, 4, 5, 6, 7, 8, 9 and 10A-10B of PCT application PCT/US18/49996 which is incorporated herein in its entirety by reference.

Alternatively, in some embodiments exemplary modified ITR sequences for use in a ceDNA vector that comprises symmetric modified ITRs, i.e., a ceDNA comprising a modified 5′ITR and a modified 3′ITR, where the modified 5′ITR and a modified 3′ITR are symmetrical or substantially symmetrical relative to each other are as shown in Table 5, which shows pairs of ITRs (modified 5′ ITR and the symmetric modified 3′ ITR). In some embodiments, the symmetrical ITR-pair is a WT-WT ITR-pair which are shown in Table 2.

The technology described herein further relates to a ceDNA vector for gene editing, where the ceDNA vector comprises a heterologous nucleic acid expression cassette can comprise, e.g., more than 4000 nucleotides, 5000 nucleotides, 10,000 nucleotides or 20,000 nucleotides, or 30,000 nucleotides, or 40,000 nucleotides or 50,000 nucleotides, or any range between about 4000-10,000 nucleotides or 10,000-50,000 nucleotides, or more than 50,000 nucleotides. The ceDNA vectors do not have the size limitations of encapsidated AAV vectors, thus enable delivery of a large-size expression cassette to provide efficient expression of transgenes. In some embodiments, the ceDNA vector is devoid of prokaryote-specific methylation.

The expression cassette can also comprise an internal ribosome entry site (IRES) and/or a 2A element. The cis-regulatory elements include, but are not limited to, a promoter, a riboswitch, an insulator, a mir-regulatable element, a post-transcriptional regulatory element, a tissue- and cell type-specific promoter and an enhancer. In some embodiments the ITR can act as the promoter for the transgene. In some embodiments, the ceDNA vector comprises additional components to regulate expression of the transgene. For example, the additional regulatory component can be a regulator switch as disclosed herein, including but not limited to a kill switch, which can kill the ceDNA infected cell, if necessary, and other inducible and/or repressible elements.

The technology described herein further provides novel methods of gene editing using the ceDNA vectors. A ceDNA vector has the capacity to be taken up into host cells, as well as to be transported into the nucleus in the absence of the AAV capsid. In addition, the ceDNA vectors described herein lack a capsid and thus avoid the immune response that can arise in response to capsid-containing vectors.

Aspects of the invention relate to methods to produce the ceDNA vectors useful for gene editing as described herein. Other embodiments relate to a ceDNA vector produced by the method provided herein. In one embodiment, the capsid free non-viral DNA vector (ceDNA vector) is obtained from a plasmid (referred to herein as a “ceDNA-plasmid”) comprising a polynucleotide expression construct template comprising in this order: a first 5′ inverted terminal repeat (e.g. AAV ITR); a heterologous nucleic acid sequence; and a 3′ ITR (e.g. AAV ITR), where the 5′ ITR and 3′ITR can be asymmetric relative to each other, or symmetric (e.g., WT-ITRs or modified symmetric ITRs) as defined herein.

The ceDNA vector disclosed herein is obtainable by a number of means that would be known to the ordinarily skilled artisan after reading this disclosure. For example, a polynucleotide expression construct template used for generating the ceDNA vectors of the present invention can be a ceDNA-plasmid (e.g. see Table 8 or FIG. 7B), a ceDNA-bacmid, and/or a ceDNA-baculovirus. In one embodiment, the ceDNA-plasmid comprises a restriction cloning site (e.g. SEQ ID NO: 7) operably positioned between the ITRs where an expression cassette comprising e.g., a promoter operatively linked to a transgene, e.g., a reporter gene and/or a therapeutic gene) can be inserted. In some embodiments, ceDNA vectors are produced from a polynucleotide template (e.g., ceDNA-plasmid, ceDNA-bacmid, ceDNA-baculovirus) containing symmetric or asymmetric ITRs (modified or WT ITRs).

In a permissive host cell, in the presence of e.g., Rep, the polynucleotide template having at least two ITRs replicates to produce ceDNA vectors. ceDNA vector production undergoes two steps: first, excision (“rescue”) of template from the template backbone (e.g. ceDNA-plasmid, ceDNA-bacmid, ceDNA-baculovirus genome etc.) via Rep proteins, and second, Rep mediated replication of the excised ceDNA vector. Rep proteins and Rep binding sites of the various AAV serotypes are well known to those of ordinary skill in the art. One of ordinary skill understands to choose a Rep protein from a serotype that binds to and replicates the nucleic acid sequence based upon at least one functional ITR. For example, if the replication competent ITR is from AAV serotype 2, the corresponding Rep would be from an AAV serotype that works with that serotype such as AAV2 ITR with AAV2 or AAV4 Rep but not AAV5 Rep, which does not. Upon replication, the covalently-closed ended ceDNA vector continues to accumulate in permissive cells and ceDNA vector is preferably sufficiently stable over time in the presence of Rep protein under standard replication conditions, e.g. to accumulate in an amount that is at least 1 pg/cell, preferably at least 2 pg/cell, preferably at least 3 pg/cell, more preferably at least 4 pg/cell, even more preferably at least 5 pg/cell.

Accordingly, one aspect of the invention relates to a process of producing a ceDNA vector for gene editing comprising the steps of: a) incubating a population of host cells (e.g. insect cells) harboring the polynucleotide expression construct template (e.g., a ceDNA-plasmid, a ceDNA-bacmid, and/or a ceDNA-baculovirus), which is devoid of viral capsid coding sequences, in the presence of a Rep protein under conditions effective and for a time sufficient to induce production of the ceDNA vector within the host cells, and wherein the host cells do not comprise viral capsid coding sequences; and b) harvesting and isolating the ceDNA vector from the host cells. The presence of Rep protein induces replication of the vector polynucleotide with a modified ITR to produce the ceDNA vector in a host cell. However, no viral particles (e.g. AAV virions) are expressed. Thus, there is no virion-enforced size limitation.

The presence of the ceDNA vector useful for gene editing is isolated from the host cells can be confirmed by digesting DNA isolated from the host cell with a restriction enzyme having a single recognition site on the ceDNA vector and analyzing the digested DNA material on denaturing and non-denaturing gels to confirm the presence of characteristic bands of linear and continuous DNA as compared to linear and non-continuous DNA.

Also provided herein in another aspect, is a method for inserting a nucleic acid sequence into a genomic safe harbor gene, the method comprising: contacting a cell with (i) a gene editing system and (ii) a homology directed repair template having homology to a genomic safe harbor gene and comprising a nucleic acid sequence encoding a protein of interest, wherein one or more components of the gene editing system are delivered to the cell by contacting the cell with a ceDNA vector composition as disclosed herein, wherein the ceDNA vector composition is a linear close-ended duplex DNA comprising flanking ITR sequences, where the ITR sequences are asymmetrical, symmetrical or substantially symmetrical relative to each other as defined herein, and at least one gene editing nucleic acid sequence having a region complementary to a genomic safe harbor gene, and wherein the method is performed under conditions and for a time sufficient to insert the nucleic acid sequence encoding the protein of interest into the genomic safe harbor gene.

In another embodiment of this aspect and all other aspects provided herein, the genomic safe harbor gene comprises an active intron close to at least one coding sequence known to express proteins at a high expression level.

In another embodiment of this aspect and all other aspects provided herein, the genomic safe harbor gene comprises a site in or near the albumin gene.

In another embodiment of this aspect and all other aspects provided herein, the genomic safe harbor gene is the AAVS1 locus.

In another embodiment of this aspect and all other aspects provided herein, the protein of interest is a receptor, a toxin, a hormone, an enzyme, or a cell surface protein. In another embodiment of this aspect and all other aspects provided herein, the protein of interest is a receptor. In another embodiment of this aspect and all other aspects provided herein, the protein of interest is a protease.

In another embodiment of this aspect and all other aspects provided herein, exemplary nonlimiting genes to be targeted, or protein of interest can be, Factor VIII (FVIII) or Factor IX (FIX). In another embodiment of this aspect and all other aspects provided herein, the method is performed in vivo for the treatment of hemophilia A, or hemophilia B. Uses of the gene editing ceDNA vectors as disclosed herein is discussed in the sections entitled “Exemplary diseases to be treated with a gene editing ceDNA” and “Additional diseases for gene editing” herein. Exemplary disease to be treated are, for example, but not limited to, Duchene Muscular Dystrophy (DMD gene), transthyretin amyloidosis (ATTR) (correct mutTTR gene), ornithine transcarbamylase deficiency (OTC deficiency), haemophilia, cystic fibrosis, sickle cell anemia, hereditary hemochromatosis, cancer, or hereditary blindness, and genes to be corrected, include but are not limited to; erythropoietin, angiostatin, endostatin, superoxide dismutase (SOD1), globin, leptin, catalase, tyrosine hydroxylase, a cytokine, cystic fibrosis transmembrane conductance regulator (CFTR), or a peptide growth factor, and the like.

In some embodiments, the present application may be defined in any of the following paragraphs:

1. A non-viral capsid-free close-ended DNA (ceDNA) vector comprising:

at least one heterologous nucleotide sequence between flanking inverted terminal repeats (ITRs), wherein at least one heterologous nucleotide sequence encodes at least one gene editing molecule.

2. The ceDNA vector of paragraph 1, wherein at least one gene editing molecule is selected from a nuclease, a guide RNA (gRNA), a guide DNA (gDNA), and an activator RNA.
3. The ceDNA vector of paragraph 2, wherein at least one gene editing molecule is a nuclease.
4. The ceDNA vector of paragraph 3, wherein the nuclease is a sequence specific nuclease.
5. The ceDNA vector of paragraph 4, wherein the sequence specific nuclease is selected from a nucleic acid-guided nuclease, zinc finger nuclease (ZFN), a meganuclease, a transcription activator-like effector nuclease (TALEN), or a megaTAL.
6. The ceDNA vector of paragraph 5, wherein the sequence specific nuclease is a nucleic acid-guided nuclease selected from a single-base editor, an RNA-guided nuclease, and a DNA-guided nuclease.
7. The ceDNA vector of paragraph 2 or paragraph 6, wherein at least one gene editing molecule is a gRNA or a gDNA.
8. The ceDNA vector of paragraph 2, 6 or 7, wherein at least one gene editing molecule is an activator RNA.
9. The ceDNA of any one of paragraphs 6-8, wherein the nucleic acid-guided nuclease is a CRISPR nuclease.
10. The ceDNA vector of paragraph 9, wherein the CRISPR nuclease is a Cas nuclease.
11. The ceDNA vector of paragraph 10, wherein the Cas nuclease is selected from Cas9, nicking Cas9 (nCas9), and deactivated Cas (dCas).
12. The ceDNA vector of paragraph 11, wherein the nCas9 contains a mutation in the HNH or RuVc domain of Cas.
13. The ceDNA vector of paragraph 11, wherein the Cas nuclease is a deactivated Cas nuclease (dCas) that complexes with a gRNA that targets a promoter region of a target gene.
14. The ceDNA vector of paragraph 13, further comprising a KRAB effector domain.
15. The ceDNA vector of paragraph 13 or paragraph 14, wherein the dCas is fused to a heterologous transcriptional activation domain that can be directed to a promoter region.
16. The ceDNA vector of paragraph 15, wherein the dCas fusion is directed to a promoter region of a target gene by a guide RNA that recruits additional transactivation domains to upregulate expression of the target gene.
17. The ceDNA vector of any one of paragraphs 13-16, wherein the dCas is S. pyogenes dCas9.
18. The ceDNA vector of any one of paragraphs 7-17, wherein the guide RNA sequence targets the promoter of a target gene and CRISPR silences the target gene (CRISPRi system).
19. The ceDNA vector of any one of paragraphs 7-17, wherein the guide RNA sequence targets the transcriptional start site of a target gene and activates the target gene (CRISPRa system).
20. The ceDNA vector of any one of paragraphs 6-19, wherein the at least one gene editing molecule comprises a first guide RNA and a second guide RNA.
21. The ceDNA vector of any one of paragraphs 7-20, wherein the gRNA targets a splice acceptor or splice donor site.
22. The ceDNA vector of paragraph 21, wherein targeting the splice acceptor or splice donor site effects non-homologous end joining (NHEJ) and correction of a defective gene.
23. The ceDNA vector of any one of paragraphs 7-22, wherein the vector encodes multiple copies of one guide RNA sequence.
24. The ceDNA vector of any one of paragraphs 1-23, wherein a first heterologous nucleotide sequence comprises a first regulatory sequence operably linked to a nucleotide sequence that encodes a nuclease.
25. The ceDNA vector of paragraph 24, wherein the first regulatory sequence comprises a promoter.
26. The ceDNA vector of paragraph 25, wherein the promoter is CAG, Pol III, U6, or H1.
27. The ceDNA vector of any one of paragraphs 24-26, wherein the first regulatory sequence comprises a modulator.
28. The ceDNA vector of paragraph 27, wherein the modulator is selected from an enhancer and a repressor.
29. The ceDNA vector of any one of paragraphs 24-28, wherein the first heterologous nucleotide sequence comprises an intron sequence upstream of the nucleotide sequence that encodes the nuclease, wherein the intron sequence comprises a nuclease cleavage site.
30. The ceDNA vector of any one of paragraphs 1-29, wherein a second heterologous nucleotide sequence comprises a second regulatory sequence operably linked to a nucleotide sequence that encodes a guide RNA.
31. The ceDNA vector of paragraph 30, wherein the second regulatory sequence comprises a promoter.
32. The ceDNA vector of paragraph 31, wherein the promoter is CAG, Pol III, U6, or H1.
33. The ceDNA vector of any one of paragraphs 30-32, wherein the second regulatory sequence comprises a modulator.
34. The ceDNA vector of paragraph 33, wherein the modulator is selected from an enhancer and a repressor.
35. The ceDNA vector of any one of paragraphs 1-34, wherein a third heterologous nucleotide sequence comprises a third regulatory sequence operably linked to a nucleotide sequence that encodes an activator RNA.
36. The ceDNA vector of paragraph 35, wherein the third regulatory sequence comprises a promoter.
37. The ceDNA vector of paragraph 36, wherein the promoter is CAG, Pol III, U6, or H1.
38. The ceDNA vector of any one of paragraphs 35-37, wherein the third regulatory sequence comprises a modulator.
39. The ceDNA vector of paragraph 38, wherein the modulator is selected from an enhancer and a repressor.
40. The ceDNA vector of any one of paragraphs 1-39, wherein the ceDNA vector comprises a 5′ homology arm and a 3′ homology arm to a target nucleic acid sequence.
41. The ceDNA vector of paragraph 40, wherein the 5′ homology arm and the 3′ homology arm are each between about 250 to 2000 bp.
42. The ceDNA vector of paragraph 40 or paragraph 41, wherein the 5′ homology arm and/or the 3′ homology arm are proximal to an ITR.
43. The ceDNA vector of any one of paragraphs 40-42, wherein at least one heterologous nucleotide sequence is between the 5′ homology arm and the 3′ homology arm.
44. The ceDNA vector of paragraph 43, wherein the at least one heterologous nucleotide sequence that is between the 5′ homology arm and the 3′ homology arm comprises a target gene.
45. The ceDNA vector of any one of paragraphs 40-44, wherein the ceDNA vector at least one heterologous nucleotide sequence that encodes a gene editing molecule is not between the 5′ homology arm and the 3′ homology arm.
46. The ceDNA vector of paragraph 45, wherein none of the heterologous nucleotide sequences that encode gene editing molecules are between the 5′ homology arm and the 3′ homology arm.
47. The ceDNA vector of any one of paragraphs 40-46, comprising a first endonuclease restriction site upstream of the 5′ homology arm and/or a second endonuclease restriction site downstream of the 3′ homology arm.
48. The ceDNA vector of paragraph 47, wherein the first endonuclease restriction site and the second endonuclease restriction site are the same restriction endonuclease sites.
49. The ceDNA vector of paragraph 47 or paragraph 48, wherein at least one endonuclease restriction site is cleaved by an endonuclease which is also encoded on the ceDNA vector.
50. The ceDNA vector of any one of paragraphs 40-49, wherein further comprises one or more poly-A sites.
51. The ceDNA vector of any one of paragraphs 40-50, comprising at least one of a transgene regulatory element and a poly-A site downstream and proximate to the 3′ homology arm and/or upstream and proximate to the 5′ homology arm.
52. The ceDNA vector of any one of paragraphs 40-51, comprising a 2A and selection marker site upstream and proximate to the 3′ homology arm.
53. The ceDNA vector of any one of paragraphs 40-52, wherein the 5′ homology arm is homologous to a nucleotide sequence upstream of a nuclease cleavage site on a chromosome.
54. The ceDNA vector of any one of paragraphs 40-53, wherein the 3′ homology arm is homologous to a nucleotide sequence downstream of a nuclease cleavage site on a chromosome.
55. The ceDNA vector of any one of paragraphs 1-54, comprising a heterologous nucleotide sequence encoding an enhancer of homologous recombination.
56. The ceDNA vector of paragraph 55, wherein the enhancer of homologous recombination is selected from SV40 late polyA signal upstream enhancer sequence, the cytomegalovirus early enhancer element, an RSV enhancer, and a CMV enhancer.
57. The ceDNA vector of any one of paragraphs 1-56, wherein at least one ITR comprises a functional terminal resolution site and a Rep binding site.
58. The ceDNA vector of any one of paragraphs 1-57, wherein the flanking ITRs are symmetric or asymmetric.
59. The ceDNA vector of paragraph 58, wherein the flanking ITRs are asymmetric, wherein at least one of the ITRs is altered from a wild-type AAV ITR sequence by a deletion, addition, or substitution that affects the overall three-dimensional conformation of the ITR.
60. The ceDNA vector of any one of paragraphs 1-59, wherein at least one heterologous nucleotide sequence is cDNA.
61. The ceDNA vector of paragraphs 1-60, wherein one or more of the flanking ITRs are derived from an AAV serotype selected from AAV1, AAV2, AAV3, AAV4, AAV5, AAV6, AAV7, AAV8, AAV9, AAV10, AAV11, and AAV12.
62. The ceDNA vector of any one of paragraphs 1-61, wherein one or more of the ITRs are synthetic.
63. The ceDNA vector of any one of paragraphs 1-62, wherein one or more of the ITRs is not a wild type ITR.
64. The ceDNA vector of any one of paragraphs 1-63, wherein one or more both of the ITRs is modified by a deletion, insertion, and/or substitution in at least one of the ITR regions selected from A, A′, B, B′, C, C′, D, and D′.
65. The ceDNA vector of paragraph 64, wherein the deletion, insertion, and/or substitution results in the deletion of all or part of a stem-loop structure normally formed by the A, A′, B, B′ C, or C′ regions.
66. The ceDNA vector of any one of paragraphs 1-58 or 56-65, wherein the ITRs are symmetrical.
67. The ceDNA vector of any one of paragraphs 1-58, 60, 61 and 66, wherein the ITRs are wild type.
68. The ceDNA vector of any one of paragraphs 1-66, wherein both ITRs are altered in a manner that results in an overall three-dimensional symmetry when the ITRs are inverted relative to each other.
69. The ceDNA vector of paragraph 68, wherein the alteration is a deletion, insertion, and/or substitution in the ITR regions selected from A, A′, B, B′, C, C′, D, and D′.
70. A method for genome editing comprising:

contacting a cell with a gene editing system, wherein one or more components of the gene editing system are delivered to the cell by contacting the cell with a non-viral capsid-free close ended DNA (ceDNA) vector comprising at least one heterologous nucleotide sequence between flanking inverted terminal repeats (ITRs), wherein at least one heterologous nucleotide sequence encodes at least one gene editing molecule.

71. The method of paragraph 70, wherein at least one gene editing molecule is selected from a nuclease, a guide RNA (gRNA), a guide DNA (gDNA), and an activator RNA.
72. The method of paragraph 71, wherein at least one gene editing molecule is a nuclease.
73. The method of paragraph 72, wherein the nuclease is a sequence specific nuclease.
74. The method of paragraph 73, wherein the sequence specific nuclease is selected from a nucleic acid-guided nuclease, zinc finger nuclease (ZFN), a meganuclease, a transcription activator-like effector nuclease (TALEN), or a megaTAL.
75. The method of paragraph 73, wherein the sequence specific nuclease is a nucleic acid-guided nuclease selected from a single-base editor, an RNA-guided nuclease, and a DNA-guided nuclease.
76. The method of paragraph 70 or 75, wherein at least one gene editing molecule is a gRNA or a gDNA.
77. The method of paragraph 70, 75 or 76, wherein at least one gene editing molecule is an activator RNA.
78. The method of any one of methods 74-77, wherein the nucleic acid-guided nuclease is a CRISPR nuclease.
79. The method of paragraph 78, wherein the CRISPR nuclease is a Cas nuclease.
80. The method of paragraph 79, wherein the Cas nuclease is selected from Cas9, nicking Cas9 (nCas9), and deactivated Cas (dCas).
81. The method of paragraph 80, wherein the nCas9 contains a mutation in the HNH or RuVc domain of Cas.
82. The method of paragraph 80, wherein the Cas nuclease is a deactivated Cas nuclease (dCas) that complexes with a gRNA that targets a promoter region of a target gene.
83. The method of paragraph 82, further comprising a KRAB effector domain.
84. The method of paragraph 82 or 83, wherein the dCas is fused to a heterologous transcriptional activation domain that can be directed to a promoter region.
85. The method of paragraph 84, wherein the dCas fusion is directed to a promoter region of a target gene by a guide RNA that recruits additional transactivation domains to upregulate expression of the target gene.
86. The method of any of paragraphs 82-85, wherein the dCas is S. pyogenes dCas9.
87. The method of any of paragraphs 78-86, wherein the guide RNA sequence targets the promoter of a target gene and CRISPR silences the target gene (CRISPRi system).
88. The method of any of paragraphs 78-86, wherein the guide RNA sequence targets the transcriptional start site of a target gene and activates the target gene (CRISPRa system).
89. The method of any of paragraphs 76-88, wherein the at least one gene editing molecule comprises a first guide RNA and a second guide RNA.
90. The method of any of paragraphs 76-89, wherein the gRNA targets a splice acceptor or splice donor site.
91. The method of paragraph 22, wherein targeting the splice acceptor or splice donor site effects non-homologous end joining (NHEJ) and correction of a defective gene.
92. The method of paragraph 76-91, wherein the vector encodes multiple copies of one guide RNA sequence.
93. The method of any of paragraphs 70-92, wherein a first heterologous nucleotide sequence comprises a first regulatory sequence operably linked to a nucleotide sequence that encodes a nuclease.
94. The method of paragraph 93, wherein the first regulatory sequence comprises a promoter.
95. The method of paragraph 94, wherein the promoter is CAG, Pol III, U6, or H1.
96. The method of any of paragraphs 93-95, wherein the first regulatory sequence comprises a modulator.
97. The method of paragraph 96, wherein the modulator is selected from an enhancer and a repressor.
98. The method of any of paragraphs 93-97, wherein the first heterologous nucleotide sequence comprises an intron sequence upstream of the nucleotide sequence that encodes the nuclease, wherein the intron sequence comprises a nuclease cleavage site.
99. The method of any of paragraphs 70-98, wherein a second heterologous nucleotide sequence comprises a second regulatory sequence operably linked to a nucleotide sequence that encodes a guide RNA.
100. The method of paragraph 99, wherein the second regulatory sequence comprises a promoter.
101. The method of paragraph 100, wherein the promoter is CAG, Pol III, U6, or H1.
102. The method of any of paragraphs 99-101, wherein the second regulatory sequence comprises a modulator.
103. The method of paragraph 102, wherein the modulator is selected from an enhancer and a repressor.
104. The method of any of paragraphs 70-103, wherein a third heterologous nucleotide sequence comprises a third regulatory sequence operably linked to a nucleotide sequence that encodes an activator RNA.
105. The method of paragraph 104, wherein the third regulatory sequence comprises a promoter.
106. The method of paragraph 105, wherein the promoter is CAG, Pol III, U6, or H1.
107. The method of paragraph 104-106, wherein the third regulatory sequence comprises a modulator.
108. The method of paragraph 107, wherein the modulator is selected from an enhancer and a repressor.
109. The method of any of paragraphs 70-108, wherein the ceDNA vector comprises a 5′ homology arm and a 3′ homology arm to a target nucleic acid sequence.
110. The method of paragraph 109, wherein the 5′ homology arm and the 3′ homology arm are each between about 250 to 2000 bp.
111. The method of paragraph 109 or 110 wherein the 5′ homology arm and/or the 3′ homology arm are proximal to an ITR.
112. The method of any of paragraphs 109-111, wherein at least one heterologous nucleotide sequence is between the 5′ homology arm and the 3′ homology arm.
113. The method of paragraph 112, wherein the at least one heterologous nucleotide sequence that is between the 5′ homology arm and the 3′ homology arm comprises a target gene.
114. The method of paragraph 109-113, wherein the ceDNA vector at least one heterologous nucleotide sequence that encodes a gene editing molecule is not between the 5′ homology arm and the 3′ homology arm.
115. The method of paragraph 114, wherein none of the heterologous nucleotide sequences that encode gene editing molecules are between the 5′ homology arm and the 3′ homology arm.
116. The method of paragraph 109-115, comprising a first endonuclease restriction site upstream of the 5′ homology arm and/or a second endonuclease restriction site downstream of the 3′ homology arm.
117. The method of paragraph 116, wherein the first endonuclease restriction site and the second endonuclease restriction site are the same restriction endonuclease sites.
118. The method of paragraph 116 or 117, wherein at least one endonuclease restriction site is cleaved by an endonuclease which is also encoded on the ceDNA vector.
119. The method of any of paragraphs 109-118, wherein further comprises one or more poly-A sites.
120. The method of any of paragraphs 109-119, comprising at least one of a transgene regulatory element and a poly-A site downstream and proximate to the 3′ homology arm and/or upstream and proximate to the 5′ homology arm.
121. The method of any of paragraphs 109-120, comprising a 2A and selection marker site upstream and proximate to the 3′ homology arm.
122. The method of any of paragraphs 109-121, wherein the 5′ homology arm is homologous to a nucleotide sequence upstream of a nuclease cleavage site on a chromosome.
123. The method of any of paragraphs 109-122, wherein the 3′ homology arm is homologous to a nucleotide sequence downstream of a nuclease cleavage site on a chromosome.
124. The method of any of paragraphs 109-123, comprising a heterologous nucleotide sequence encoding an enhancer of homologous recombination.
125. The method of paragraph 124, wherein the enhancer of homologous recombination is selected from SV40 late polyA signal upstream enhancer sequence, the cytomegalovirus early enhancer element, an RSV enhancer, and a CMV enhancer.
126. The method of any of paragraphs 70-125, wherein at least one ITR comprises a functional terminal resolution site and a Rep binding site.
127. The method of any of paragraphs 70-126, wherein the flanking ITRs are symmetric or asymmetric.
128. The method of paragraph 127, wherein the flanking ITRs are asymmetric, wherein at least one of the ITRs is altered from a wild-type AAV ITR sequence by a deletion, addition, or substitution that affects the overall three-dimensional conformation of the ITR.
129. The method of any of paragraphs 70-128, wherein at least one heterologous nucleotide sequence is cDNA.
130. The method of any of paragraphs 70-129, wherein one or more of the flanking ITRs are derived from an AAV serotype selected from AAV1, AAV2, AAV3, AAV4, AAV5, AAV6, AAV7, AAV8, AAV9, AAV10, AAV11, and AAV12.
131. The method of any of paragraphs 70-130, wherein one or more of the ITRs are synthetic.
132. The method of any of paragraphs 70-131, wherein one or more of the ITRs is not a wild type ITR.
133. The method of any of paragraphs 70-132, wherein one or more both of the ITRs is modified by a deletion, insertion, and/or substitution in at least one of the ITR regions selected from A, A′, B, B′, C, C′, D, and D′.
134. The method of paragraph 133, wherein the deletion, insertion, and/or substitution results in the deletion of all or part of a stem-loop structure normally formed by the A, A′, B, B′ C, or C′ regions.
135. The method of any of paragraphs 70-127 or 129-134, wherein the ITRs are symmetrical.
136. The method of any one of paragraphs 70-127 or 129-130, wherein the ITRs are wild type.
137. The method of any of paragraphs 70-136, wherein both ITRs are altered in a manner that results in an overall three-dimensional symmetry when the ITRs are inverted relative to each other.
138. The method of paragraph 137, wherein the alteration is a deletion, insertion, and/or substitution in the ITR regions selected from A, A′, B, B′, C, C′, D, and D′.
139. The method of any of paragraphs 70-138, wherein the cell contacted is a eukaryotic cell.
140. The method of any of paragraphs 84-139, wherein the CRISPR nuclease is codon optimized for expression in the eukaryotic cell.
141. The method of any of paragraphs 84-140, wherein the Cas protein is codon optimized for expression in the eukaryotic cell.
142. A method of genome editing comprising administering to a cell an effective amount of a non-viral capsid-free closed ended DNA (ceDNA vector) of any one of paragraphs 1-69, under conditions suitable and for a time sufficient to edit a target gene.
143. The method of any of paragraphs 113-142, wherein the target gene is gene targeted using one or more guide RNA sequences and edited by homology directed repair (HDR) in the presence of a HDR donor template.
144. The method of any of paragraphs 142-143, wherein the target gene is targeted using one guide RNA sequence and the target gene is edited by non-homologous end joining (NHEJ).
145. The method of any of paragraphs 70-144, wherein the method is performed in vivo to correct a single nucleotide polymorphism (SNP) associated with a disease.
146. The method of paragraph 145, wherein the disease comprises sickle cell anemia, hereditary hemochromatosis or cancer hereditary blindness.
147. The method of any of paragraphs 70-146, wherein at least 2 different Cas proteins are present in the ceDNA vector, and wherein one of the Cas protein is catalytically inactive (Cas-i), and wherein the guide RNA associated with the Cas-I targets the promoter of the target cell, and wherein the DNA coding for the Cas-I is under the control of an inducible promoter so that it can turn-off the expression of the target gene at a desired time.
148. A method for editing a single nucleotide base pair in a target gene of a cell, the method comprising contacting a cell with a CRISPR/Cas gene editing system, wherein one or more components of the CRISPR/Cas gene editing system are delivered to the cell by contacting the cell with a non-viral capsid-free close-ended DNA (ceDNA) vector composition, and

wherein the Cas protein expressed from the ceDNA vector is catalytically inactive and is fused to a base editing moiety,

wherein the method is performed under conditions and for a time sufficient to modulate expression of the target gene.

149. The method of paragraph 148, wherein the ceDNA vector is a ceDNA vector of any of paragraphs 1-69.
150. The method of paragraph 148, wherein the base editing moiety comprises a single-strand-specific cytidine deaminase, a uracil glycosylase inhibitor, or a tRNA adenosine deaminase.
151. The method of paragraph 148, wherein the catalytically inactive Cas protein is dCas9.
152. The method of any of paragraphs 70-151, wherein the cell is a T cell, or CD34⁺.
153. The method of any of paragraphs 70-152, wherein the target gene encodes for a programmed death protein (PD1), cytotoxic T-lymphocyte-associated antigen 4 (CTLA4), or tumor necrosis factor-α (TNF-α).
154. The method of any of paragraphs 70-153, further comprising administering the cells produced to a subject in need thereof.
155. The method of paragraph 154, wherein the subject in need thereof has a genetic disease, viral infection, bacterial infection, cancer, or autoimmune disease.
156. A method of modulating expression of two or more target genes in a cell comprising: introducing into the cell:

(i) a first composition comprising a vector that comprises: flanking terminal repeat (TR) sequences, and a nucleic acid sequence encoding at least two guide RNAs complementary to two or more target genes, wherein the vector is a non-viral capsid free closed ended DNA (ceDNA) vector,

(ii) a second composition comprising a vector that comprises: flanking terminal repeat (TR) sequences and a nucleic acid sequence encoding at least two catalytically inactive DNA endonucleases that each associate with a guide RNA and bind to the two or more target genes, wherein the vector is a non-viral capsid free closed ended DNA (ceDNA) vector, and

(iii) a third composition comprising a vector that comprises flanking terminal repeat (TR) sequences, and a nucleic acid sequence encoding at least two transcriptional regulator proteins or domains, wherein the vector is a non-viral capsid free closed ended DNA (ceDNA) vector and

wherein the at least two guide RNAs, the at least two catalytically inactive RNA-guided endonucleases and the at least two transcriptional regulator proteins or domains are expressed in the cell,

wherein two or more co-localization complexes form between a guide RNA, a catalytically inactive RNA-guided endonuclease, a transcriptional regulator protein or domain and a target gene, and

wherein the transcriptional regulator protein or domain regulates expression of the at least two target genes.

157. The method of paragraph 156, wherein the ceDNA vector of the first composition is a ceDNA vector of any of paragraphs 1-69, the ceDNA vector of the second composition is a ceDNA vector of any of paragraphs 1-69, and the third composition is a ceDNA vector of any of paragraphs 1-69.
158. A method for inserting a nucleic acid sequence into a genomic safe harbor gene, the method comprising: contacting a cell with (i) a gene editing system and (ii) a homology directed repair template having homology to a genomic safe harbor gene and comprising a nucleic acid sequence encoding a protein of interest,

wherein one or more components of the gene editing system are delivered to the cell by contacting the cell with a non-viral capsid-free close-ended DNA (ceDNA) vector composition, wherein the ceDNA nucleic acid vector composition comprises at least one heterologous nucleotide sequence between flanking inverted terminal repeats (ITRs), wherein at least one heterologous nucleotide sequence encodes at least one gene editing molecule, and

wherein the method is performed under conditions and for a time sufficient to insert the nucleic acid sequence encoding the protein of interest into the genomic safe harbor gene.

159. The method of paragraph 158, wherein the ceDNA vector is a ceDNA vector of any of paragraphs 1-69.
160. The method of paragraph 158, wherein the genomic safe harbor gene comprises an active intron close to at least one coding sequence known to express proteins at a high expression level.
161. The method of paragraph 158, wherein the genomic safe harbor gene comprises a site in or near any one of: the albumin gene, CCR5 gene, AAVS1 locus.
162. The method of any of paragraphs 158-161, wherein the protein of interest is a receptor, a toxin, a hormone, an enzyme, or a cell surface protein.
163. The method of any of paragraphs 162, wherein, the protein of interest is a secreted protein.
164. The method of paragraph 163, wherein the protein of interest comprises Factor VIII (FVIII) or Factor IX (FIX).
165. The method of paragraph 164, wherein the method is performed in vivo for the treatment of hemophilia A, or hemophilia B.
166. A method of inserting a donor sequence at a predetermined insertion site on a chromosome in a host cell, comprising: introducing into the host cell the ceDNA vector of paragraphs 1-69, wherein the donor sequence is inserted into the chromosome at or adjacent to the insertion site through homologous recombination.
167. A method of generating a genetically modified animal comprising a donor sequence inserted at a predetermined insertion site on the chromosome of the animal, comprising a) generating a cell with the donor sequence inserted at the predetermined insertion site on the chromosome according to paragraph 167; and b) introducing the cell generated by a) into a carrier animal to produce the genetically modified animal.
168. The method of paragraph 167, wherein the cell is a zygote or a pluripotent stem cell.
169. A genetically modified animal generated by the method of paragraph 168.
170. The genetically modified animal of paragraph 169, wherein the animal is a non-human animal.
171. A kit for inserting a donor sequence at an insertion site on a chromosome in a cell, comprising: a) a first non-viral capsid-free close-ended DNA (ceDNA) vector comprising:

two AAV inverted terminal repeat (ITR); and

a first nucleotide sequence comprising a 5′ homology arm, a donor sequence, and a 3′ homology arm, wherein the donor sequence has gene editing functionality; and

(a) a second ceDNA vector comprising:

at least one AAV ITR; and

a nucleotide sequence encoding at least one gene editing molecule,

wherein in the first ceDNA vector, the 5′ homology arm is homologous to a sequence upstream of a cleavage site for gene editing molecule on the chromosome and wherein the 3′ homology arm is homologous to a sequence downstream of the gene editing molecule cleavage site on the chromosome; and wherein the 5′ homology arm or the 3′ homology arm are proximal to the ITR.

172. The method of paragraph 171, wherein the gene editing molecule is a nuclease.
173. The method of paragraph 172, wherein the nuclease is a sequence specific nuclease.
174. The method of any of paragraphs 171-173, wherein the first ceDNA vector is a ceDNA vector of any of paragraphs 1, 40-56, 57-69.
175. The method of any of paragraphs 171-173, wherein the second ceDNA vector is a ceDNA vector of any of paragraphs 1-39 or paragraphs 57-69.
176. A method of inserting a donor sequence at a predetermined insertion site on a chromosome in a host cell, comprising:

a) introducing into the host cell a first non-viral capsid-free close-ended DNA (ceDNA) vector having at least one inverted terminal repeat (ITR), wherein the ceDNA vector comprises a first linear nucleic acid comprising a 5′ homology arm, a donor sequence, and a 3′ homology arm; and

b) introducing into the host cell a second ceDNA vector comprising least one heterologous nucleotide sequence between flanking inverted terminal repeats (ITRs), wherein at least one heterologous nucleotide sequence encodes at least one gene editing molecule that cleaves the chromosome at or adjacent to the insertion site, wherein the donor sequence is inserted into the chromosome at or adjacent to the insertion site through homologous recombination.

177. The method of paragraph 176, wherein the gene editing molecule is a nuclease.
178. The method of paragraph 177, wherein the nuclease is a sequence specific nuclease.
179. The method of any of paragraphs 176-178, wherein the first ceDNA vector is a ceDNA vector of any of paragraphs 1, 40-56, 57-69.
180. The method of any of paragraphs 176-179, wherein the second ceDNA vector is a ceDNA vector of any of paragraphs 1-39 or paragraphs 57-69.
181. The method of any of paragraphs 179-180, wherein the second ceDNA vector further comprises a third nucleotide sequence encoding a guide sequence recognizing the insertion site.
182. A cell containing a ceDNA vector of any of paragraphs 1-69.
183. A composition comprising a vector of any of paragraphs 1-69 and a lipid.
184. The composition of paragraph 184, wherein the lipid is a lipid nanoparticle (LNP).
185. A kit comprising a composition of paragraph 183 or 184 or a cell of paragraph 182.

In some embodiments, one aspect of the technology described herein relates to a non-viral capsid-free DNA vector with covalently-closed ends (ceDNA vector), wherein the ceDNA vector comprises at least one heterologous nucleotide sequence, operably positioned between asymmetric inverted terminal repeat sequences (asymmetric ITRs), wherein at least one of the asymmetric ITRs comprises a functional terminal resolution site and a Rep binding site, and optionally the heterologous nucleic acid sequence encodes a transgene, and wherein the vector is not in a viral capsid.

These and other aspects of the invention are described in further detail below.

### DESCRIPTION OF DRAWINGS

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIG. 1A illustrates an exemplary structure of a ceDNA vector comprising asymmetric ITRs for gene editing. In this embodiment, the exemplary ceDNA vector comprises an expression cassette containing CAG promoter, WPRE, and BGHpA. An open reading frame (ORF) encoding a luciferase transgene is inserted into the cloning site (R3/R4) between the CAG promoter and WPRE. The expression cassette is flanked by two inverted terminal repeats (ITRs)—the wild-type AAV2 ITR on the upstream (5′-end) and the modified ITR on the downstream (3′-end) of the expression cassette, therefore the two ITRs flanking the expression cassette are asymmetric with respect to each other.

FIG. 1B illustrates an exemplary structure of a ceDNA vector comprising asymmetric ITRs for gene editing with an expression cassette containing CAG promoter, WPRE, and BGHpA. An open reading frame (ORF) encoding Luciferase transgene is inserted into the cloning site between CAG promoter and WPRE. The expression cassette is flanked by two inverted terminal repeats (ITRs)—a modified ITR on the upstream (5′-end) and a wild-type ITR on the downstream (3′-end) of the expression cassette.

FIG. 1C illustrates an exemplary structure of a ceDNA vector for gene editing comprising asymmetric ITRs, with an expression cassette containing an enhancer/promoter, an open reading frame (ORF) for insertion of a transgene which is a gene editing molecule, or a gene editing nucleic acid sequence, a post transcriptional element (WPRE), and a polyA signal. An open reading frame (ORF) allows insertion of a transgene which is a gene editing molecule, the gene editing nucleic acid sequence into the cloning site between CAG promoter and WPRE. The expression cassette is flanked by two inverted terminal repeats (ITRs) that are asymmetrical with respect to each other; a modified ITR on the upstream (5′-end) and a modified ITR on the downstream (3′-end) of the expression cassette, where the 5′ ITR and the 3′ITR are both modified ITRs but have different modifications (i.e., they do not have the same modifications).

FIG. 1D illustrates an exemplary structure of a ceDNA vector for gene editing comprising symmetric modified ITRs, or substantially symmetrical modified ITRs as defined herein, with an expression cassette containing CAG promoter, WPRE, and BGHpA. An open reading frame (ORF) encoding Luciferase transgene is inserted into the cloning site between CAG promoter and WPRE. The expression cassette is flanked by two modified inverted terminal repeats (ITRs), where the 5′ modified ITR and the 3′ modified ITR are symmetrical or substantially symmetrical.

FIG. 1E illustrates an exemplary structure of a ceDNA vector for gene editing comprising symmetric modified ITRs, or substantially symmetrical modified ITRs as defined herein, with an expression cassette containing an enhancer/promoter, an open reading frame (ORF) for insertion of a transgene which is a gene editing molecule, or a gene editing nucleic acid sequence, a post transcriptional element (WPRE), and a polyA signal. An open reading frame (ORF) allows insertion of a transgene which is a gene editing molecule, the gene editing nucleic acid sequence into the cloning site between CAG promoter and WPRE. The expression cassette is flanked by two modified inverted terminal repeats (ITRs), where the 5′ modified ITR and the 3′ modified ITR are symmetrical or substantially symmetrical.

FIG. 1F illustrates an exemplary structure of a ceDNA vector for gene editing comprising symmetric WT-ITRs, or substantially symmetrical WT-ITRs as defined herein, with an expression cassette containing CAG promoter, WPRE, and BGHpA. An open reading frame (ORF) encoding Luciferase transgene is inserted into the cloning site between CAG promoter and WPRE. The expression cassette is flanked by two wild type inverted terminal repeats (WT-ITRs), where the 5′ WT-ITR and the 3′ WT ITR are symmetrical or substantially symmetrical.

FIG. 1G illustrates an exemplary structure of a ceDNA vector for gene editing comprising symmetric modified ITRs, or substantially symmetrical modified ITRs as defined herein, with an expression cassette containing an enhancer/promoter, an open reading frame (ORF) for insertion of a transgene which is a gene editing molecule, or a gene editing nucleic acid sequence, a post transcriptional element (WPRE), and a polyA signal. An open reading frame (ORF) allows insertion of a transgene which is a gene editing molecule, the gene editing nucleic acid sequence into the cloning site between CAG promoter and WPRE. The expression cassette is flanked by two wild type inverted terminal repeats (WT-ITRs), where the 5′ WT-ITR and the 3′ WT ITR are symmetrical or substantially symmetrical.

FIG. 2A provides the T-shaped stem-loop structure of a wild-type left ITR of AAV2 (SEQ ID NO: 538) with identification of A-A′ arm, B-B′ arm, C-C′ arm, two Rep binding sites (RBE and RBE′) and also shows the terminal resolution site (trs). The RBE contains a series of 4 duplex tetramers that are believed to interact with either Rep 78 or Rep 68. In addition, the RBE′ is also believed to interact with Rep complex assembled on the wild-type ITR or mutated ITR in the construct. The D and D′ regions contain transcription factor binding sites and other conserved structure. FIG. 2B shows proposed Rep-catalyzed nicking and ligating activities in a wild-type left ITR (SEQ ID NO: 539), including the T-shaped stem-loop structure of the wild-type left ITR of AAV2 with identification of A-A′ arm, B-B′ arm, C-C′ arm, two Rep Binding sites (RBE and RBE′) and also shows the terminal resolution site (trs), and the D and D′ region comprising several transcription factor binding sites and other conserved structure.

FIG. 3A provides the primary structure (polynucleotide sequence) (left) and the secondary structure (right) of the RBE-containing portions of the A-A′ arm, and the C-C′ and B-B′ arm of the wild type left AAV2 ITR (SEQ ID NO: 540). FIG. 3B shows an exemplary mutated ITR (also referred to as a modified ITR) sequence for the left ITR. Shown is the primary structure (left) and the predicted secondary structure (right) of the RBE portion of the A-A′ arm, the C arm and B-B′ arm of an exemplary mutated left ITR (ITR-1, left) (SEQ ID NO: 113). FIG. 3C shows the primary structure (left) and the secondary structure (right) of the RBE-containing portion of the A-A′ loop, and the B-B′ and C-C′ arms of wild type right AAV2 ITR (SEQ ID NO: 541). FIG. 3D shows an exemplary right modified ITR. Shown is the primary structure (left) and the predicted secondary structure (right) of the RBE containing portion of the A-A′ arm, the B-B′ and the C arm of an exemplary mutant right ITR (ITR-1, right) (SEQ ID NO: 114). Any combination of left and right ITR (e.g., AAV2 ITRs or other viral serotype or synthetic ITRs) can be used as taught herein. Each of FIGS. 3A-3D polynucleotide sequences refer to the sequence used in the plasmid or bacmid/baculovirus genome used to produce the ceDNA as described herein. Also included in each of FIGS. 3A-3D are corresponding ceDNA secondary structures inferred from the ceDNA vector configurations in the plasmid or bacmid/baculovirus genome and the predicted Gibbs free energy values.

FIG. 4A is a schematic illustrating an upstream process for making baculovirus infected insect cells (BIICs) that are useful in the production of ceDNA in the process described in the schematic in FIG. 4B. FIG. 4B is a schematic of an exemplary method of ceDNA production and FIG. 4C illustrates a biochemical method and process to confirm ceDNA vector production. FIG. 4D and FIG. 4E are schematic illustrations describing a process for identifying the presence of ceDNA in DNA harvested from cell pellets obtained during the ceDNA production processes in FIG. 4B. FIG. 4E shows DNA having a non-continuous structure. The ceDNA can be cut by a restriction endonuclease, having a single recognition site on the ceDNA vector, and generate two DNA fragments with different sizes (1 kb and 2 kb) in both neutral and denaturing conditions. FIG. 4E also shows a ceDNA having a linear and continuous structure. The ceDNA vector can be cut by the restriction endonuclease, and generate two DNA fragments that migrate as lkb and 2 kb in neutral conditions, but in denaturing conditions, the stands remain connected and produce single strands that migrate as 2 kb and 4 kb. FIG. 4D shows schematic expected bands for an exemplary ceDNA either left uncut or digested with a restriction endonuclease and then subjected to electrophoresis on either a native gel or a denaturing gel. The leftmost schematic is a native gel, and shows multiple bands suggesting that in its duplex and uncut form ceDNA exists in at least monomeric and dimeric states, visible as a faster-migrating smaller monomer and a slower-migrating dimer that is twice the size of the monomer. The schematic second from the left shows that when ceDNA is cut with a restriction endonuclease, the original bands are gone and faster-migrating (e.g., smaller) bands appear, corresponding to the expected fragment sizes remaining after the cleavage. Under denaturing conditions, the original duplex DNA is single-stranded and migrates as a species twice as large as observed on native gel because the complementary strands are covalently linked Thus in the second schematic from the right, the digested ceDNA shows a similar banding distribution to that observed on native gel, but the bands migrate as fragments twice the size of their native gel counterparts. The rightmost schematic shows that uncut ceDNA under denaturing conditions migrates as a single-stranded open circle, and thus the observed bands are twice the size of those observed under native conditions where the circle is not open. In this figure “kb” is used to indicate relative size of nucleotide molecules based, depending on context, on either nucleotide chain length (e.g., for the single stranded molecules observed in denaturing conditions) or number of basepairs (e.g., for the double-stranded molecules observed in native conditions).

FIG. 5 is an exemplary picture of a denaturing gel running examples of ceDNA vectors with (+) or without (−) digestion with endonucleases (EcoRI for ceDNA construct 1 and 2; BamH1 for ceDNA construct 3 and 4; Spel for ceDNA construct 5 and 6; and Xhol for ceDNA construct 7 and 8). Sizes of bands highlighted with an asterisk were determined and provided on the bottom of the picture.

FIG. 6A is an exemplary Rep-bacmid in the pFBDLSR plasmid comprising the nucleic acid sequences for Rep proteins Rep52 and Rep78. This exemplary Rep-bacmid comprises: IE1 promoter fragment (SEQ ID NO:66); Rep78 nucleotide sequence, including Kozak sequence (SEQ ID NO:67), polyhedron promoter sequence for Rep52 (SEQ ID NO:68) and Rep58 nucleotide sequence, starting with Kozak sequence gccgccacc) (SEQ ID NO:69). FIG. 6B is a schematic of an exemplary ceDNA-plasmid-1, with the wt-L ITR, CAG promoter, luciferase transgene, WPRE and polyadenylation sequence, and mod-R ITR.

FIG. 7A shows predicted structures of the RBE-containing portion of the A-A′ arm and modified B-B′ arm and/or modified C-C′ arm of exemplary modified right ITRs listed in Table 4A. FIG. 7B shows predicted structures of the RBE-containing portion of the A-A′ arm and modified C-C′ arm and/or modified B-B′ arm of exemplary modified left ITRs listed in Table 4B. The structures shown are the predicted lowest free energy structure. Color code: red=>99% probability; orange=99%-95% probability; beige=95-90% probability; dark green 90%-80%; bright green=80%-70%; light blue=70%-60%; dark blue 60%-50% and pink=<50%.

FIG. 8 is a schematic illustration of a ceDNA vector in accordance with the present disclosure.

FIG. 9 is a schematic illustration of a ceDNA vector in accordance with the present disclosure that is different than FIG. 20.

FIGS. 10A-10F depict a schematic view of ceDNA vectors in accordance with the present disclosure.

FIG. 11 is a schematic view of ceDNA vectors in accordance with the present disclosure. Enh: enhancer, Pro=promoter, intron=synthetic or natural occurring intron with splice donor and acceptor seq, NLS=nuclear localization signal nuclease=ORF for Cas9, ZFN, Talen, or other endonuclease sequences. Filled arrows represent the sgRNA seq. (single guide-RNA target sequences (e.g., 4) are selected using freely available software/algorithm picked out and validated experimentally), open arrows represent alternative sgRNA sequences.

FIG. 12 is a schematic view of ceDNA vectors in accordance with the present disclosure.

FIG. 13 is a schematic view of ceDNA vectors in accordance with the present disclosure.

FIG. 14 is a schematic view of expression cassettes for expressing sgRNA.

FIG. 15 is a schematic illustration of a ceDNA vector in accordance with the present disclosure that is different than FIGS. 20 and 21.

FIG. 16 is a schematic illustration of a ceDNA vector in accordance with the present disclosure. Three of the ceDNA vectors comprise with 5′ and 3′ homology arms and promoter-less transgenes suitable for insertion into Albumin. Also depicted is a ceDNA with 5′ and 3′ homology arms that comprises a promoter driven transgene, e.g., a reporter gene that can be inserted into any safe harbor site. A target region where insertion does not cause significant negative effects. A genomic safe harbor site in a given genome (e.g., human genome) can be determined using techniques known in the art and described in, for example, Papapetrou, E R & Schambach, A. Molecular Therapy 24(4):678-684 (2016) or Sadelain et al. Nature Reviews Cancer 12:51-58 (2012), the contents of each of which are incorporated herein by reference in their entirety.

FIG. 17 is a schematic diagram and sequence of a target center for an Albumin mouse locus and donor template encoding FIX. FIG. 17 discloses SEQ ID NO: 835.

FIG. 18A and FIG. 18B are schematic diagram and sequence of a target center for an Albumin mouse locus homology arms and example guide RNA locations (FIG. 18A), and guide RNAS (FIG. 18B). FIGS. 18A and 18B dicloses SEQ ID NOS 835-841, respectively, in order of appearance.

FIG. 19 provided herein is a schematic showing exemplary work-flow methods for gene editing experimental protocols useful with the methods and compositions described herein, including (i) cell delivery of an expression vector, (ii) design of gRNA, (iii) cell culture methods and optimization, (iv) Cas9 RNP assembly, (v) ceDNA vectors comprising homology directed repair templates, and (vi) detection of successful gene editing.

### DETAILED DESCRIPTION OF THE INVENTION

### I. Definitions

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), Fields Virology, 6^{th} Edition, published by Lippincott Williams & Wilkins, Philadelphia, Pa., USA (2013), Knipe, D. M. and Howley, P. M. (ed.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, the terms “heterologous nucleotide sequence” and “transgene” are used interchangeably and refer to a nucleic acid of interest (other than a nucleic acid encoding a capsid polypeptide) that is incorporated into and may be delivered and expressed by a ceDNA vector as disclosed herein.

As used herein, the terms “expression cassette” and “transcription cassette” are used interchangeably and refer to a linear stretch of nucleic acids that includes a transgene that is operably linked to one or more promoters or other regulatory sequences sufficient to direct transcription of the transgene, but which does not comprise capsid-encoding sequences, other vector sequences or inverted terminal repeat regions. An expression cassette may additionally comprise one or more cis-acting sequences (e.g., promoters, enhancers, or repressors), one or more introns, and one or more post-transcriptional regulatory elements.

The terms “polynucleotide” and “nucleic acid,” used interchangeably herein, refer to a polymeric form of nucleotides of any length, either ribonucleotides or deoxyribonucleotides. Thus, this term includes single, double, or multi-stranded DNA or RNA, genomic DNA, cDNA, DNA-RNA hybrids, or a polymer including purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” and “nucleic acid” should be understood to include, as applicable to the embodiments being described, single-stranded (such as sense or antisense) and double-stranded polynucleotides.

The term “nucleic acid construct” as used herein refers to a nucleic acid molecule, either single- or double-stranded, which is isolated from a naturally occurring gene or which is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present disclosure. An “expression cassette” includes a DNA coding sequence operably linked to a promoter.

By “hybridizable” or “complementary” or “substantially complementary” it is meant that a nucleic acid (e.g., RNA) includes a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine (A) pairing with thymidine (T), adenine (A) pairing with uracil (U), and guanine (G) pairing with cytosine (C). In addition, it is also known in the art that for hybridization between two RNA molecules (e.g., dsRNA), guanine (G) base pairs with uracil (U). For example, G/U base-pairing is partially responsible for the degeneracy (i.e., redundancy) of the genetic code in the context of tRNA anti-codon base-pairing with codons in mRNA. In the context of this disclosure, a guanine (G) of a protein-binding segment (dsRNA duplex) of a subject DNA-targeting RNA molecule is considered complementary to a uracil (U), and vice versa. As such, when a G/U base-pair can be made at a given nucleotide position a protein-binding segment (dsRNA duplex) of a subject DNA-targeting RNA molecule, the position is not considered to be non-complementary, but is instead considered to be complementary.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

A DNA sequence that “encodes” a particular RNA or protein gene product is a DNA nucleic acid sequence that is transcribed into the particular RNA and/or protein. A DNA polynucleotide may encode an RNA (mRNA) that is translated into protein, or a DNA polynucleotide may encode an RNA that is not translated into protein (e.g., tRNA, rRNA, or a DNA-targeting RNA; also called “non-coding” RNA or “ncRNA”).

As used herein, the term “gene editing molecule” refers to one or more of a protein or a nucleic acid encoding for a protein, wherein the protein is selected from the group comprising a transposase, a nuclease, an integrase, a guide RNA (gRNA), a guide DNA, a ribonucleoprotein (RNP), or an activator RNA. A nuclease gene editing molecule is a protein having nuclease activity, with nonlimiting examples including: a CRISPR protein (Cas), CRISPR associated protein 9 (Cas9); a type IIS restriction enzyme; a transcription activator-like effector nuclease (TALEN); and a zinc finger nuclease (ZFN), a meganuclease, engineered site-specific nucleases or deactivated CAS for CRISPRi or CRISPRa systems. The gene editing molecule can also comprise a DNA-binding domain and a nuclease. In certain embodiments, the gene editing molecule comprises a DNA-binding domain and a nuclease. In certain embodiments, the DNA-binding domain comprises a guide RNA. In certain embodiments, the DNA-binding domain comprises a DNA-binding domain of a TALEN. In certain embodiments at least one gene editing molecule comprises one or more transposable element(s). In certain embodiments, the one or more transposable element(s) comprise a circular DNA. In certain embodiments, the one or more transposable element(s) comprise a plasmid vector or a minicircle DNA vector. In certain embodiments, the DNA-binding domain comprises a DNA-binding domain of a zinc-finger nuclease. In certain embodiments at least one gene editing molecule comprises one or more transposable element(s). In certain embodiments, the one or more transposable element(s) comprise a linear DNA. The linear recombinant and non-naturally occurring DNA sequence encoding a transposon may be produced in vitro. Linear recombinant and non-naturally occurring DNA sequences of the disclosure may be a product of restriction digest of a circular DNA. In certain embodiments, the circular DNA is a plasmid vector or a minicircle DNA vector. Linear recombinant and non-naturally occurring DNA sequences of the disclosure may be a product of a polymerase chain reaction (PCR). Linear recombinant and non-naturally occurring DNA sequences of the disclosure may be a double-stranded Doggybone™ DNA sequence. Doggybone™ DNA sequences of the disclosure may be produced by an enzymatic process that solely encodes an antigen expression cassette, comprisin antigen, promoter, poly-A tail and telomeric ends.

As used herein, the term “gene editing functionality” refers to the insertion, deletion or replacement of DNA at a specific site in the genome with a loss or gain of function. The insertion, deletion or replacement of DNA at a specific site can be accomplished e.g. by homology-directed repair (HDR) or non-homologous end joining (NHEJ), or single base change editing. In some embodiments, a donor template is used, for example for HDR, such that a desired sequence within the donor template is inserted into the genome by a homologous recombination event. In one embodiment, a “donor template” or “repair template” comprises two homology arms (e.g., a 5′ homology arm and a 3′ homology arm) flanking on either side of a donor sequence comprising a desired mutation or insertion in the nucleic acid sequence to be introduced into the host genome. The 5′ and 3′ homology arms are substantially homologous to the genomic sequence of the target gene at the site of endonuclease mediated cutting. The 3′ homology arm is generally immediately downstream of the protospacer adjacent motif (PAM) site where the endonuclease cuts (e.g., a double stranded DNA cut), or in some embodiments, nicks the DNA.

As used herein, the term “gene editing system” refers to the minimum components necessary to effect genome editing in a cell. For example, a zinc finger nuclease or TALEN system may only require expression of the endonuclease fused to a nucleic acid complementary to the sequence of a target gene, whereas for a CRISPR/Cas gene editing system the minimum components may require e.g., a Cas endonuclease and a guide RNA. The gene editing system can be encoded on a single ceDNA vector or multiple vectors, as desired. Those of skill in the art will readily understand the component(s) necessary for a gene editing system.

As used herein, the term “base editing moiety” refers to an enzyme or enzyme system that can alter a single nucleotide in a sequence, for example, a cytosine/guanine nucleotide pair “G/C” to an adenine and thymine “T”/uridine “U” nucleotide pair (A/T,U) (see e.g., Shevidi et al. Dev Dyn 31 (2017) PMID:28857338; Kyoungmi et al. Nature Biotechnology 35:435-437 (2017), the contents of each of which are incorporated herein by reference in their entirety) or an adenine/thymine “A/T” nucleotide pair to a guanine/cytosine “G/C” nucleotide pair (see e.g., Gaudelli et al. Nature (2017), in press doi:10.1038/nature24644, the contents of which are incorporated herein by reference in its entirety).

As used herein, the term “genomic safe harbor gene” or “safe harbor gene” refers to a gene or loci that a nucleic acid sequence can be inserted such that the sequence can integrate and function in a predictable manner (e.g., express a protein of interest) without significant negative consequences to endogenous gene activity, or the promotion of cancer. In some embodiments, a safe harbor gene is also a loci or gene where an inserted nucleic acid sequence can be expressed efficiently and at higher levels than a non-safe harbor site.

As used herein, the term “gene delivery” means a process by which foreign DNA is transferred to host cells for applications of gene therapy.

As used herein, the term “CRISPR” stands for Clustered Regularly Interspaced Short Palindromic Repeats, which are the hallmark of a bacterial defense system that forms the basis for CRISPR-Cas9 genome editing technology.

As used herein, the term “zinc finger” means a small protein structural motif that is characterized by the coordination of one or more zinc ions, in order to stabilize the fold.

As used herein, the term “homologous recombination” means a type of genetic recombination in which nucleotide sequences are exchanged between two similar or identical molecules of DNA. Homologous recombination also produces new combinations of DNA sequences. These new combinations of DNA represent genetic variation. Homologous recombination is also used in horizontal gene transfer to exchange genetic material between different strains and species of viruses.

As used herein, the term “terminal repeat” or “TR” includes any viral terminal repeat or synthetic sequence that comprises at least one minimal required origin of replication and a region comprising a palindrome hairpin structure. A Rep-binding sequence (“RBS”) (also referred to as RBE (Rep-binding element)) and a terminal resolution site (“TRS”) together constitute a “minimal required origin of replication” and thus the TR comprises at least one RBS and at least one TRS. TRs that are the inverse complement of one another within a given stretch of polynucleotide sequence are typically each referred to as an “inverted terminal repeat” or “ITR”. In the context of a virus, ITRs mediate replication, virus packaging, integration and provirus rescue. As was unexpectedly found in the invention herein, TRs that are not inverse complements across their full length can still perform the traditional functions of ITRs, and thus the term ITR is used herein to refer to a TR in a ceDNA genome or ceDNA vector that is capable of mediating replication of ceDNA vector. It will be understood by one of ordinary skill in the art that in complex ceDNA vector configurations more than two ITRs or asymmetric ITR pairs may be present. The ITR can be an AAV ITR or a non-AAV ITR, or can be derived from an AAV ITR or a non-AAV ITR. For example, the ITR can be derived from the family Parvoviridae, which encompasses parvoviruses and dependoviruses (e.g., canine parvovirus, bovine parvovirus, mouse parvovirus, porcine parvovirus, human parvovirus B-19), or the SV40 hairpin that serves as the origin of SV40 replication can be used as an ITR, which can further be modified by truncation, substitution, deletion, insertion and/or addition. Parvoviridae family viruses consist of two subfamilies Parvovirinae, which infect vertebrates, and Densovirinae, which infect invertebrates. Dependoparvoviruses include the viral family of the adeno-associated viruses (AAV) which are capable of replication in vertebrate hosts including, but not limited to, human, primate, bovine, canine, equine and ovine species. For convenience herein, an ITR located 5′ to (upstream of) an expression cassette in a ceDNA vector is referred to as a “5′ ITR” or a “left ITR”, and an ITR located 3′ to (downstream of) an expression cassette in a ceDNA vector is referred to as a “3′ ITR” or a “right ITR”.

A “wild-type ITR” or “WT-ITR” refers to the sequence of a naturally occurring ITR sequence in an AAV or other dependovirus that retains, e.g., Rep binding activity and Rep nicking ability. The nucleotide sequence of a WT-ITR from any AAV serotype may slightly vary from the canonical naturally occurring sequence due to degeneracy of the genetic code or drift, and therefore WT-ITR sequences encompassed for use herein include WT-ITR sequences as result of naturally occurring changes taking place during the production process (e.g., a replication error).

As used herein, the term “substantially symmetrical WT-ITRs” or a “substantially symmetrical WT-ITR pair” refers to a pair of WT-ITRs within a single ceDNA genome or ceDNA vector that are both wild type ITRs that have an inverse complement sequence across their entire length. For example, an ITR can be considered to be a wild-type sequence, even if it has one or more nucleotides that deviate from the canonical naturally occurring sequence, so long as the changes do not affect the properties and overall three-dimensional structure of the sequence. In some aspects, the deviating nucleotides represent conservative sequence changes. As one non-limiting example, a sequence that has at least 95%, 96%, 97%, 98%, or 99% sequence identity to the canonical sequence (as measured, e.g., using BLAST at default settings), and also has a symmetrical three-dimensional spatial organization to the other WT-ITR such that their 3D structures are the same shape in geometrical space. The substantially symmetrical WT-ITR has the same A, C-C′ and B-B′ loops in 3D space. A substantially symmetrical WT-ITR can be functionally confirmed as WT by determining that it has an operable Rep binding site (RBE or RBE′) and terminal resolution site (trs) that pairs with the appropriate Rep protein. One can optionally test other functions, including transgene expression under permissive conditions.

As used herein, the phrases of “modified ITR” or “mod-ITR” or “mutant ITR” are used interchangeably herein and refer to an ITR that has a mutation in at least one or more nucleotides as compared to the WT-ITR from the same serotype. The mutation can result in a change in one or more of A, C, C′, B, B′ regions in the ITR, and can result in a change in the three-dimensional spatial organization (i.e. its 3D structure in geometric space) as compared to the 3D spatial organization of a WT-ITR of the same serotype.

As used herein, the term “asymmetric ITRs” also referred to as “asymmetric ITR pairs” refers to a pair of ITRs within a single ceDNA genome or ceDNA vector that are not inverse complements across their full length. As one non-limiting example, an asymmetric ITR pair does not have a symmetrical three-dimensional spatial organization to their cognate ITR such that their 3D structures are different shapes in geometrical space. Stated differently, an asymmetrical ITR pair have the different overall geometric structure, i.e., they have different organization of their A, C-C′ and B-B′ loops in 3D space (e.g., one ITR may have a short C-C′ arm and/or short B-B′ arm as compared to the cognate ITR). The difference in sequence between the two ITRs may be due to one or more nucleotide addition, deletion, truncation, or point mutation. In one embodiment, one ITR of the asymmetric ITR pair may be a wild-type AAV ITR sequence and the other ITR a modified ITR as defined herein (e.g., a non-wild-type or synthetic ITR sequence). In another embodiment, neither ITRs of the asymmetric ITR pair is a wild-type AAV sequence and the two ITRs are modified ITRs that have different shapes in geometrical space (i.e., a different overall geometric structure). In some embodiments, one mod-ITRs of an asymmetric ITR pair can have a short C-C′ arm and the other ITR can have a different modification (e.g., a single arm, or a short B-B′ arm etc.) such that they have different three-dimensional spatial organization as compared to the cognate asymmetric mod-ITR.

As used herein, the term “symmetric ITRs” refers to a pair of ITRs within a single ceDNA genome or ceDNA vector that are mutated or modified relative to wild-type dependoviral ITR sequences and are inverse complements across their full length. Neither ITRs are wild type ITR AAV2 sequences (i.e., they are a modified ITR, also referred to as a mutant ITR), and can have a difference in sequence from the wild type ITR due to nucleotide addition, deletion, substitution, truncation, or point mutation. For convenience herein, an ITR located 5′ to (upstream of) an expression cassette in a ceDNA vector is referred to as a “5′ ITR” or a “left ITR”, and an ITR located 3′ to (downstream of) an expression cassette in a ceDNA vector is referred to as a “3′ ITR” or a “right ITR”.

As used herein, the terms “substantially symmetrical modified-ITRs” or a “substantially symmetrical mod-ITR pair” refers to a pair of modified-ITRs within a single ceDNA genome or ceDNA vector that are both that have an inverse complement sequence across their entire length. For example, the a modified ITR can be considered substantially symmetrical, even if it has some nucleotide sequences that deviate from the inverse complement sequence so long as the changes do not affect the properties and overall shape. As one non-limiting example, a sequence that has at least 85%, 90%, 95%, 96%, 97%, 98%, or 99% sequence identity to the canonical sequence (as measured using BLAST at default settings), and also has a symmetrical three-dimensional spatial organization to their cognate modified ITR such that their 3D structures are the same shape in geometrical space. Stated differently, a substantially symmetrical modified-ITR pair have the same A, C-C′ and B-B′ loops organized in 3D space. In some embodiments, the ITRs from a mod-ITR pair may have different reverse complement nucleotide sequences but still have the same symmetrical three-dimensional spatial organization—that is both ITRs have mutations that result in the same overall 3D shape. For example, one ITR (e.g., 5′ ITR) in a mod-ITR pair can be from one serotype, and the other ITR (e.g., 3′ ITR) can be from a different serotype, however, both can have the same corresponding mutation (e.g., if the 5′ITR has a deletion in the C region, the cognate modified 3′ITR from a different serotype has a deletion at the corresponding position in the C′ region), such that the modified ITR pair has the same symmetrical three-dimensional spatial organization. In such embodiments, each ITR in a modified ITR pair can be from different serotypes (e.g. AAV1, 2, 3, 4, 5, 6, 7, 8, 9, 10, 11, and 12) such as the combination of AAV2 and AAV6, with the modification in one ITR reflected in the corresponding position in the cognate ITR from a different serotype. In one embodiment, a substantially symmetrical modified ITR pair refers to a pair of modified ITRs (mod-ITRs) so long as the difference in nucleotide sequences between the ITRs does not affect the properties or overall shape and they have substantially the same shape in 3D space. As a non-limiting example, a mod-ITR that has at least 95%, 96%, 97%, 98% or 99% sequence identity to the canonical mod-ITR as determined by standard means well known in the art such as BLAST (Basic Local Alignment Search Tool), or BLASTN at default settings, and also has a symmetrical three-dimensional spatial organization such that their 3D structure is the same shape in geometric space. A substantially symmetrical mod-ITR pair has the same A, C-C′ and B-B′ loops in 3D space, e.g., if a modified ITR in a substantially symmetrical mod-ITR pair has a deletion of a C-C′ arm, then the cognate mod-ITR has the corresponding deletion of the C-C′ loop and also has a similar 3D structure of the remaining A and B-B′ loops in the same shape in geometric space of its cognate mod-ITR.

The term “flanking” refers to a relative position of one nucleic acid sequence with respect to another nucleic acid sequence. Generally, in the sequence ABC, B is flanked by A and C. The same is true for the arrangement AxBxC. Thus, a flanking sequence precedes or follows a flanked sequence but need not be contiguous with, or immediately adjacent to the flanked sequence. In one embodiment, the term flanking refers to terminal repeats at each end of the linear duplex ceDNA vector.

As used herein, the term “ceDNA genome” refers to an expression cassette that further incorporates at least one inverted terminal repeat region. A ceDNA genome may further comprise one or more spacer regions. In some embodiments the ceDNA genome is incorporated as an intermolecular duplex polynucleotide of DNA into a plasmid or viral genome.

As used herein, the term “ceDNA spacer region” refers to an intervening sequence that separates functional elements in the ceDNA vector or ceDNA genome. In some embodiments, ceDNA spacer regions keep two functional elements at a desired distance for optimal functionality. In some embodiments, ceDNA spacer regions provide or add to the genetic stability of the ceDNA genome within e.g., a plasmid or baculovirus. In some embodiments, ceDNA spacer regions facilitate ready genetic manipulation of the ceDNA genome by providing a convenient location for cloning sites and the like. For example, in certain aspects, an oligonucleotide “polylinker” containing several restriction endonuclease sites, or a non-open reading frame sequence designed to have no known protein (e.g., transcription factor) binding sites can be positioned in the ceDNA genome to separate the cis-acting factors, e.g., inserting a 6mer, 12mer, 18mer, 24mer, 48mer, 86mer, 176mer, etc. between the terminal resolution site and the upstream transcriptional regulatory element. Similarly, the spacer may be incorporated between the polyadenylation signal sequence and the 3′-terminal resolution site.

As used herein, the terms “Rep binding site, “Rep binding element, “RBE” and “RBS” are used interchangeably and refer to a binding site for Rep protein (e.g., AAV Rep 78 or AAV Rep 68) which upon binding by a Rep protein permits the Rep protein to perform its site-specific endonuclease activity on the sequence incorporating the RBS. An RBS sequence and its inverse complement together form a single RBS. RBS sequences are known in the art, and include, for example, 5′-GCGCGCTCGCTCGCTC-3′ (SEQ ID NO: 531), an RBS sequence identified in AAV2. Any known RBS sequence may be used in the embodiments of the invention, including other known AAV RBS sequences and other naturally known or synthetic RBS sequences. Without being bound by theory it is thought that he nuclease domain of a Rep protein binds to the duplex nucleotide sequence GCTC, and thus the two known AAV Rep proteins bind directly to and stably assemble on the duplex oligonucleotide, 5′-(GCGC)(GCTC)(GCTC)(GCTC)-3′ (SEQ ID NO: 531). In addition, soluble aggregated conformers (i.e., undefined number of inter-associated Rep proteins) dissociate and bind to oligonucleotides that contain Rep binding sites. Each Rep protein interacts with both the nitrogenous bases and phosphodiester backbone on each strand. The interactions with the nitrogenous bases provide sequence specificity whereas the interactions with the phosphodiester backbone are non- or less-sequence specific and stabilize the protein-DNA complex.

As used herein, the terms “terminal resolution site” and “TRS” are used interchangeably herein and refer to a region at which Rep forms a tyrosine-phosphodiester bond with the 5′ thymidine generating a 3′ OH that serves as a substrate for DNA extension via a cellular DNA polymerase, e.g., DNA pol delta or DNA pol epsilon. Alternatively, the Rep-thymidine complex may participate in a coordinated ligation reaction. In some embodiments, a TRS minimally encompasses a non-base-paired thymidine. In some embodiments, the nicking efficiency of the TRS can be controlled at least in part by its distance within the same molecule from the RBS. When the acceptor substrate is the complementary ITR, then the resulting product is an intramolecular duplex. TRS sequences are known in the art, and include, for example, 5′-GGTTGA-3′ (SEQ ID NO: 45), the hexanucleotide sequence identified in AAV2. Any known TRS sequence may be used in the embodiments of the invention, including other known AAV TRS sequences and other naturally known or synthetic TRS sequences such as AGTT (SEQ ID NO: 46), GGTTGG (SEQ ID NO: 47), AGTTGG (SEQ ID NO: 48), AGTTGA (SEQ ID NO: 49), and other motifs such as RRTTRR (SEQ ID NO: 50).

As used herein, the term “ceDNA-plasmid” refers to a plasmid that comprises a ceDNA genome as an intermolecular duplex.

As used herein, the term “ceDNA-bacmid” refers to an infectious baculovirus genome comprising a ceDNA genome as an intermolecular duplex that is capable of propagating in E. coli as a plasmid, and so can operate as a shuttle vector for baculovirus.

As used herein, the term “ceDNA-baculovirus” refers to a baculovirus that comprises a ceDNA genome as an intermolecular duplex within the baculovirus genome.

As used herein, the terms “ceDNA-baculovirus infected insect cell” and “ceDNA-BIIC” are used interchangeably, and refer to an invertebrate host cell (including, but not limited to an insect cell (e.g., an Sf9 cell)) infected with a ceDNA-baculovirus.

As used herein, the terms “closed-ended DNA vector”, “ceDNA vector” and “ceDNA” are used interchangeably and refer to a non-virus capsid-free DNA vector with at least one covalently-closed end (i.e., an intramolecular duplex). In some embodiments, the ceDNA comprises two covalently-closed ends.

As defined herein, “reporters” refer to proteins that can be used to provide detectable read-outs. Reporters generally produce a measurable signal such as fluorescence, color, or luminescence. Reporter protein coding sequences encode proteins whose presence in the cell or organism is readily observed. For example, fluorescent proteins cause a cell to fluoresce when excited with light of a particular wavelength, luciferases cause a cell to catalyze a reaction that produces light, and enzymes such as β-galactosidase convert a substrate to a colored product. Exemplary reporter polypeptides useful for experimental or diagnostic purposes include, but are not limited to β-lactamase, β-galactosidase (LacZ), alkaline phosphatase (AP), thymidine kinase (TK), green fluorescent protein (GFP) and other fluorescent proteins, chloramphenicol acetyltransferase (CAT), luciferase, and others well known in the art.

As used herein, the term “effector protein” refers to a polypeptide that provides a detectable read-out, either as, for example, a reporter polypeptide, or more appropriately, as a polypeptide that kills a cell, e.g., a toxin, or an agent that renders a cell susceptible to killing with a chosen agent or lack thereof. Effector proteins include any protein or peptide that directly targets or damages the host cell's DNA and/or RNA. For example, effector proteins can include, but are not limited to, a restriction endonuclease that targets a host cell DNA sequence (whether genomic or on an extrachromosomal element), a protease that degrades a polypeptide target necessary for cell survival, a DNA gyrase inhibitor, and a ribonuclease-type toxin. In some embodiments, the expression of an effector protein controlled by a synthetic biological circuit as described herein can participate as a factor in another synthetic biological circuit to thereby expand the range and complexity of a biological circuit system's responsiveness.

Transcriptional regulators refer to transcriptional activators and repressors that either activate or repress transcription of a gene of interest. Promoters are regions of nucleic acid that initiate transcription of a particular gene Transcriptional activators typically bind nearby to transcriptional promoters and recruit RNA polymerase to directly initiate transcription. Repressors bind to transcriptional promoters and sterically hinder transcriptional initiation by RNA polymerase. Other transcriptional regulators may serve as either an activator or a repressor depending on where they bind and cellular and environmental conditions. Non-limiting examples of transcriptional regulator classes include, but are not limited to homeodomain proteins, zinc-finger proteins, winged-helix (forkhead) proteins, and leucine-zipper proteins.

As used herein, a “repressor protein” or “inducer protein” is a protein that binds to a regulatory sequence element and represses or activates, respectively, the transcription of sequences operatively linked to the regulatory sequence element. Preferred repressor and inducer proteins as described herein are sensitive to the presence or absence of at least one input agent or environmental input. Preferred proteins as described herein are modular in form, comprising, for example, separable DNA-binding and input agent-binding or responsive elements or domains.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutically active substances is well known in the art. Supplementary active ingredients can also be incorporated into the compositions. The phrase “pharmaceutically-acceptable” refers to molecular entities and compositions that do not produce a toxic, an allergic, or similar untoward reaction when administered to a host.

As used herein, an “input agent responsive domain” is a domain of a transcription factor that binds to or otherwise responds to a condition or input agent in a manner that renders a linked DNA binding fusion domain responsive to the presence of that condition or input. In one embodiment, the presence of the condition or input results in a conformational change in the input agent responsive domain, or in a protein to which it is fused, that modifies the transcription-modulating activity of the transcription factor.

The term “in vivo” refers to assays or processes that occur in or within an organism, such as a multicellular animal. In some of the aspects described herein, a method or use can be said to occur “in vivo” when a unicellular organism, such as a bacterium, is used. The term “ex vivo” refers to methods and uses that are performed using a living cell with an intact membrane that is outside of the body of a multicellular animal or plant, e.g., explants, cultured cells, including primary cells and cell lines, transformed cell lines, and extracted tissue or cells, including blood cells, among others. The term “in vitro” refers to assays and methods that do not require the presence of a cell with an intact membrane, such as cellular extracts, and can refer to the introducing of a programmable synthetic biological circuit in a non-cellular system, such as a medium not comprising cells or cellular systems, such as cellular extracts.

The term “promoter,” as used herein, refers to any nucleic acid sequence that regulates the expression of another nucleic acid sequence by driving transcription of the nucleic acid sequence, which can be a heterologous target gene encoding a protein or an RNA. Promoters can be constitutive, inducible, repressible, tissue-specific, or any combination thereof. A promoter is a control region of a nucleic acid sequence at which initiation and rate of transcription of the remainder of a nucleic acid sequence are controlled. A promoter can also contain genetic elements at which regulatory proteins and molecules can bind, such as RNA polymerase and other transcription factors. In some embodiments of the aspects described herein, a promoter can drive the expression of a transcription factor that regulates the expression of the promoter itself. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the expression of transgenes in the ceDNA vectors disclosed herein. A promoter sequence may be bounded at its 3′ terminus by the transcription initiation site and extends upstream (5′ direction) to include the minimum number of bases or elements necessary to initiate transcription at levels detectable above background.

The term “enhancer” as used herein refers to a cis-acting regulatory sequence (e.g., 50-1,500 base pairs) that binds one or more proteins (e.g., activator proteins, or transcription factor) to increase transcriptional activation of a nucleic acid sequence. Enhancers can be positioned up to 1,000,000 base pars upstream of the gene start site or downstream of the gene start site that they regulate. An enhancer can be positioned within an intronic region, or in the exonic region of an unrelated gene.

A promoter can be said to drive expression or drive transcription of the nucleic acid sequence that it regulates. The phrases “operably linked,” “operatively positioned,” “operatively linked,” “under control,” and “under transcriptional control” indicate that a promoter is in a correct functional location and/or orientation in relation to a nucleic acid sequence it regulates to control transcriptional initiation and/or expression of that sequence. An “inverted promoter,” as used herein, refers to a promoter in which the nucleic acid sequence is in the reverse orientation, such that what was the coding strand is now the non-coding strand, and vice versa. Inverted promoter sequences can be used in various embodiments to regulate the state of a switch. In addition, in various embodiments, a promoter can be used in conjunction with an enhancer.

A promoter can be one naturally associated with a gene or sequence, as can be obtained by isolating the 5′ non-coding sequences located upstream of the coding segment and/or exon of a given gene or sequence. Such a promoter can be referred to as “endogenous.” Similarly, in some embodiments, an enhancer can be one naturally associated with a nucleic acid sequence, located either downstream or upstream of that sequence.

In some embodiments, a coding nucleic acid segment is positioned under the control of a “recombinant promoter” or “heterologous promoter,” both of which refer to a promoter that is not normally associated with the encoded nucleic acid sequence it is operably linked to in its natural environment. A recombinant or heterologous enhancer refers to an enhancer not normally associated with a given nucleic acid sequence in its natural environment. Such promoters or enhancers can include promoters or enhancers of other genes; promoters or enhancers isolated from any other prokaryotic, viral, or eukaryotic cell; and synthetic promoters or enhancers that are not “naturally occurring,” i.e., comprise different elements of different transcriptional regulatory regions, and/or mutations that alter expression through methods of genetic engineering that are known in the art. In addition to producing nucleic acid sequences of promoters and enhancers synthetically, promoter sequences can be produced using recombinant cloning and/or nucleic acid amplification technology, including PCR, in connection with the synthetic biological circuits and modules disclosed herein (see, e.g., U.S. Pat. Nos. 4,683,202, 5,928,906, each incorporated herein by reference). Furthermore, it is contemplated that control sequences that direct transcription and/or expression of sequences within non-nuclear organelles such as mitochondria, chloroplasts, and the like, can be employed as well.

As described herein, an “inducible promoter” is one that is characterized by initiating or enhancing transcriptional activity when in the presence of, influenced by, or contacted by an inducer or inducing agent. An “inducer” or “inducing agent,” as defined herein, can be endogenous, or a normally exogenous compound or protein that is administered in such a way as to be active in inducing transcriptional activity from the inducible promoter. In some embodiments, the inducer or inducing agent, i.e., a chemical, a compound or a protein, can itself be the result of transcription or expression of a nucleic acid sequence (i.e., an inducer can be an inducer protein expressed by another component or module), which itself can be under the control or an inducible promoter. In some embodiments, an inducible promoter is induced in the absence of certain agents, such as a repressor. Examples of inducible promoters include but are not limited to, tetracycline, metallothionine, ecdysone, mammalian viruses (e.g., the adenovirus late promoter; and the mouse mammary tumor virus long terminal repeat (MMTV-LTR)) and other steroid-responsive promoters, rapamycin responsive promoters and the like.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g., DNA-targeting RNA) or a coding sequence (e.g., site-directed modifying polypeptide, or Cas9/Csn1 polypeptide) and/or regulate translation of an encoded polypeptide.

“Operably linked” refers to a juxtaposition wherein the components so described are in a relationship permitting them to function in their intended manner. For instance, a promoter is operably linked to a coding sequence if the promoter affects its transcription or expression. An “expression cassette” includes an exogenous DNA sequence that is operably linked to a promoter or other regulatory sequence sufficient to direct transcription of the transgene in the ceDNA vector. Suitable promoters include, for example, tissue specific promoters. Promoters can also be of AAV origin.

The term “subject” as used herein refers to a human or animal, to whom treatment, including prophylactic treatment, with the ceDNA vector according to the present invention, is provided. Usually the animal is a vertebrate such as, but not limited to a primate, rodent, domestic animal or game animal. Primates include but are not limited to, chimpanzees, cynomologous monkeys, spider monkeys, and macaques, e.g., Rhesus. Rodents include mice, rats, woodchucks, ferrets, rabbits and hamsters. Domestic and game animals include, but are not limited to, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments of the aspects described herein, the subject is a mammal, e.g., a primate or a human. A subject can be male or female. Additionally, a subject can be an infant or a child. In some embodiments, the subject can be a neonate or an unborn subject, e.g., the subject is in utero. Preferably, the subject is a mammal. The mammal can be a human, non-human primate, mouse, rat, dog, cat, horse, or cow, but is not limited to these examples. Mammals other than humans can be advantageously used as subjects that represent animal models of diseases and disorders. In addition, the methods and compositions described herein can be used for domesticated animals and/or pets. A human subject can be of any age, gender, race or ethnic group, e.g., Caucasian (white), Asian, African, black, African American, African European, Hispanic, Mideastern, etc. In some embodiments, the subject can be a patient or other subject in a clinical setting. In some embodiments, the subject is already undergoing treatment. In some embodiments, the subject is an embryo, a fetus, neonate, infant, child, adolescent, or adult. In some embodiments, the subject is a human fetus, human neonate, human infant, human child, human adolescent, or human adult. In some embodiments, the subject is an animal embryo, or non-human embryo or non-human primate embryo. In some embodiments, the subject is a human embryo.

As used herein, the term “host cell”, includes any cell type that is susceptible to transformation, transfection, transduction, and the like with a nucleic acid construct or ceDNA expression vector of the present disclosure. As non-limiting examples, a host cell can be an isolated primary cell, pluripotent stem cells, CD34⁺ cells), induced pluripotent stem cells, or any of a number of immortalized cell lines (e.g., HepG2 cells). Alternatively, a host cell can be an in situ or in vivo cell in a tissue, organ or organism.

The term “exogenous” refers to a substance present in a cell other than its native source. The term “exogenous” when used herein can refer to a nucleic acid (e.g., a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell.

The term “sequence identity” refers to the relatedness between two nucleotide sequences. For purposes of the present disclosure, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows: (Identical Deoxyribonucleotides.times.100)/(Length of Alignment-Total Number of Gaps in Alignment). The length of the alignment is preferably at least 10 nucleotides, preferably at least 25 nucleotides more preferred at least 50 nucleotides and most preferred at least 100 nucleotides.

The term “homology” or “homologous” as used herein is defined as the percentage of nucleotide residues in the homology arm that are identical to the nucleotide residues in the corresponding sequence on the target chromosome, after aligning the sequences and introducing gaps, if necessary, to achieve the maximum percent sequence identity. Alignment for purposes of determining percent nucleotide sequence homology can be achieved in various ways that are within the skill in the art, for instance, using publicly available computer software such as BLAST, BLAST-2, ALIGN, ClustalW2 or Megalign (DNASTAR) software. Those skilled in the art can determine appropriate parameters for aligning sequences, including any algorithms needed to achieve maximal alignment over the full length of the sequences being compared. In some embodiments, a nucleic acid sequence (e.g., DNA sequence), for example of a homology arm of a repair template, is considered “homologous” when the sequence is at least 70%, at least 75%, at least 80%, at least 85%, at least 90%, at least 91%, at least 92%, at least 93%, at least 94%, at least 95%, at least 96%, at least 97%, at least 98%, at least 99%, or more, identical to the corresponding native or unedited nucleic acid sequence (e.g., genomic sequence) of the host cell.

As used herein, a “homology arm” refers to a polynucleotide that is suitable to target a donor sequence to a genome through homologous recombination. Typically, two homology arms flank the donor sequence, wherein each homology arm comprises genomic sequences upstream and downstream of the loci of integration.

As used herein, “a donor sequence” refers to a polynucleotide that is to be inserted into, or used as a repair template for, a host cell genome. The donor sequence can comprise the modification which is desired to be made during gene editing. The sequence to be incorporated can be introduced into the target nucleic acid molecule via homology directed repair at the target sequence, thereby causing an alteration of the target sequence from the original target sequence to the sequence comprised by the donor sequence. Accordingly, the sequence comprised by the donor sequence can be, relative to the target sequence, an insertion, a deletion, an indel, a point mutation, a repair of a mutation, etc. The donor sequence can be, e.g., a single-stranded DNA molecule; a double-stranded DNA molecule; a DNA/RNA hybrid molecule; and a DNA/modRNA (modified RNA) hybrid molecule. In one embodiment, the donor sequence is foreign to the homology arms. The editing can be RNA as well as DNA editing. The donor sequence can be endogenous to or exogenous to the host cell genome, depending upon the nature of the desired gene editing.

The term “heterologous,” as used herein, means a nucleotide or polypeptide sequence that is not found in the native nucleic acid or protein, respectively. For example, in a chimeric Cas9/Csn1 protein, the RNA-binding domain of a naturally-occurring bacterial Cas9/Csn1 polypeptide (or a variant thereof) may be fused to a heterologous polypeptide sequence (i.e. a polypeptide sequence from a protein other than Cas9/Csn1 or a polypeptide sequence from another organism). The heterologous polypeptide sequence may exhibit an activity (e.g., enzymatic activity) that will also be exhibited by the chimeric Cas9/Csn1 protein (e.g., methyltransferase activity, acetyltransferase activity, kinase activity, ubiquitinating activity, etc.). A heterologous nucleic acid sequence may be linked to a naturally-occurring nucleic acid sequence (or a variant thereof) (e.g., by genetic engineering) to generate a chimeric nucleotide sequence encoding a chimeric polypeptide. As another example, in a fusion variant Cas9 site-directed polypeptide, a variant Cas9 site-directed polypeptide may be fused to a heterologous polypeptide (i.e. a polypeptide other than Cas9), which exhibits an activity that will also be exhibited by the fusion variant Cas9 site-directed polypeptide. A heterologous nucleic acid sequence may be linked to a variant Cas9 site-directed polypeptide (e.g., by genetic engineering) to generate a nucleotide sequence encoding a fusion variant Cas9 site-directed polypeptide.

A “vector” or “expression vector” is a replicon, such as plasmid, bacmid, phage, virus, virion, or cosmid, to which another DNA segment, i.e. an “insert”, may be attached so as to bring about the replication of the attached segment in a cell. A vector can be a nucleic acid construct designed for delivery to a host cell or for transfer between different host cells. As used herein, a vector can be viral or non-viral in origin and/or in final form, however for the purpose of the present disclosure, a “vector” generally refers to a ceDNA vector, as that term is used herein. The term “vector” encompasses any genetic element that is capable of replication when associated with the proper control elements and that can transfer gene sequences to cells. In some embodiments, a vector can be an expression vector or recombinant vector.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g., 5′ untranslated (5′UTR) or “leader” sequences and 3′ UTR or “trailer” sequences, as well as intervening sequences (introns) between individual coding segments (exons).

By “recombinant vector” is meant a vector that includes a heterologous nucleic acid sequence, or “transgene” that is capable of expression in vivo. It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

The terms “correcting”, “genome editing” and “restoring” as used herein refers to changing a mutant gene that encodes a truncated protein or no protein at all, such that a full-length functional or partially full-length functional protein expression is obtained. Correcting or restoring a mutant gene may include replacing the region of the gene that has the mutation or replacing the entire mutant gene with a copy of the gene that does not have the mutation with a repair mechanism such as homology-directed repair (HDR). Correcting or restoring a mutant gene may also include repairing a frameshift mutation that causes a premature stop codon, an aberrant splice acceptor site or an aberrant splice donor site, by generating a double stranded break in the gene that is then repaired using non-homologous end joining (NHEJ). NHEJ may add or delete at least one base pair during repair which may restore the proper reading frame and eliminate the premature stop codon. Correcting or restoring a mutant gene may also include disrupting an aberrant splice acceptor site or splice donor sequence. Correcting or restoring a mutant gene may also include deleting a non-essential gene segment by the simultaneous action of two nucleases on the same DNA strand in order to restore the proper reading frame by removing the DNA between the two nuclease target sites and repairing the DNA break by NHEJ.

The phrase “genetic disease” as used herein refers to a disease, partially or completely, directly or indirectly, caused by one or more abnormalities in the genome, especially a condition that is present from birth. The abnormality may be a mutation, an insertion or a deletion. The abnormality may affect the coding sequence of the gene or its regulatory sequence. The genetic disease may be, but not limited to DMD, hemophilia, cystic fibrosis, Huntington's chorea, familial hypercholesterolemia (LDL receptor defect), hepatoblastoma, Wilson's disease, congenital hepatic porphyria, inherited disorders of hepatic metabolism, Lesch Nyhan syndrome, sickle cell anemia, thalassaemias, xeroderma pigmentosum, Fanconi's anemia, retinitis pigmentosa, ataxia telangiectasia, Bloom's syndrome, retinoblastoma, and Tay-Sachs disease.

The phrase “non-homologous end joining (NHEJ) pathway” as used herein refers to a pathway that repairs double-strand breaks in DNA by directly ligating the break ends without the need for a homologous template. The template-independent re-ligation of DNA ends by NHEJ is a stochastic, error-prone repair process that introduces random micro-insertions and micro-deletions (indels) at the DNA breakpoint. This method may be used to intentionally disrupt, delete, or alter the reading frame of targeted gene sequences. NHEJ typically uses short homologous DNA sequences called microhomologies to guide repair. These microhomologies are often present in single-stranded overhangs on the end of double-strand breaks. When the overhangs are perfectly compatible, NHEJ usually repairs the break accurately, yet imprecise repair leading to loss of nucleotides may also occur, but is much more common when the overhangs are not compatible “Nuclease mediated NHEJ” as used herein refers to NHEJ that is initiated after a nuclease, such as a cas9 or other nuclease, cuts double stranded DNA. In a CRISPR/CAS system NHEJ can be targeted by using a single guide RNA sequence.

The phrase “homology-directed repair” or “HDR” as used interchangeably herein refers to a mechanism in cells to repair double strand DNA lesions when a homologous piece of DNA is present in the nucleus. HDR uses a donor DNA template to guide repair and may be used to create specific sequence changes to the genome, including the targeted addition of whole genes. If a donor template is provided along with the site specific nuclease, such as with a CRISPR/Cas9-based systems, then the cellular machinery will repair the break by homologous recombination, which is enhanced several orders of magnitude in the presence of DNA cleavage. When the homologous DNA piece is absent, non-homologous end joining may take place instead. In a CRISPR/Cas system one guide RNA, or two different guide RNAS can be used for HDR.

The phrase “repeat variable diresidue” or “RVD” as used interchangeably herein refers to a pair of adjacent amino acid residues within a DNA recognition motif (also known as “RVD module”), which includes 33-35 amino acids, of a TALE DNA-binding domain. The RVD determines the nucleotide specificity of the RVD module. RVD modules may be combined to produce an RVD array. The “RVD array length” as used herein refers to the number of RVD modules that corresponds to the length of the nucleotide sequence within the TALEN target region that is recognized by a TALEN, i.e., the binding region.

The terms “site-specific nuclease” or “sequence specific nuclease” as used herein refers to an enzyme capable of specifically recognizing and cleaving DNA sequences. The site-specific nuclease may be engineered. Examples of engineered site-specific nucleases include zinc finger nucleases (ZFNs), TAL effector nucleases (TALENs), and CRISPR/Cas-based systems, that use various natural and unnatural Cas enzymes.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Other than in the operating examples, or where otherwise indicated, all numbers expressing quantities of ingredients or reaction conditions used herein should be understood as modified in all instances by the term “about.” The term “about” when used in connection with percentages can mean±1%. The present invention is further explained in detail by the following examples, but the scope of the invention should not be limited thereto.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

### II. ceDNA Vector for Gene Editing

Embodiments of the invention are based on methods and compositions comprising close ended linear duplexed (ceDNA) vectors that can express a transgene which is a gene editing molecule in a host cell (e.g., a transgene is a nuclease such as ZFN, TALEN, Cas; one or more guide RNA; CRISPR; a ribonucleoprotein (RNP), or any combination thereof) and result in more efficient genome editing. The ceDNA vectors described herein are not limited by size, thereby permitting, for example, expression of all of the components necessary for a gene editing system from a single vector (e.g., a CRISPR/Cas gene editing system (e.g., a Cas9 or modified Cas9 enzyme, a guide RNA and/or a homology directed repair template), or for a TALEN or Zinc Finger system). However, it is also contemplated that having only one or two of such components encoded on a single vector, while the remaining component(s) can be expressed on a separate ceDNA vector or e.g. a traditional plasmid.

One aspect herein relates to a novel ceDNA vector for DNA knock-in method(s), e.g., for the introduction of one or more exogenous donor sequences into a specific target site on a cellular chromosome with high efficiency. In addition to the use of one or more ceDNA vector for gene editing, where the ceDNA vector comprises ITR sequences selected from any of: (i) at least one WT ITR and at least one modified AAV inverted terminal repeat (mod-ITR) (e.g., asymmetric modified ITRs); (ii) two modified ITRs where the mod-ITR pair have a different three-dimensional spatial organization with respect to each other (e.g., asymmetric modified ITRs), or (iii) symmetrical or substantially symmetrical WT-WT ITR pair, where each WT-ITR has the same three-dimensional spatial organization, or (iv) symmetrical or substantially symmetrical modified ITR pair, where each mod-ITR has the same three-dimensional spatial organization, the methods and compositions as disclosed herein may further include a delivery system, such as but not limited to, a liposome nanoparticle delivery system. Nonlimiting exemplary liposome nanoparticle systems encompassed for use are disclosed herein. In some aspects, the disclosure provides for a lipid nanoparticle comprising ceDNA for gene editing and an ionizable lipid. For example, a lipid nanoparticle formulation that is made and loaded with a gene editing ceDNA obtained by the process is disclosed in International Application PCT/US2018/050042, filed on Sep. 7, 2018, which is incorporated herein.

Provided herein are novel non-viral, capsid-free ceDNA molecules with covalently-closed ends (ceDNA). These non-viral capsid free ceDNA molecules can be produced in permissive host cells from an expression construct (e.g., a ceDNA-plasmid, a ceDNA-bacmid, a ceDNA-baculovirus, or an integrated cell-line) containing a heterologous gene (transgene) positioned between two different inverted terminal repeat (ITR) sequences, where the ITRs are different with respect to each other. In some embodiments, one of the ITRs is modified by deletion, insertion, and/or substitution as compared to a wild-type ITR sequence (e.g. AAV ITR); and at least one of the ITRs comprises a functional terminal resolution site (trs) and a Rep binding site. The ceDNA vector is preferably duplex, e.g self-complementary, over at least a portion of the molecule, such as the expression cassette (e.g. ceDNA is not a double stranded circular molecule). The ceDNA vector has covalently closed ends, and thus is resistant to exonuclease digestion (e.g. exonuclease I or exonuclease III), e.g. for over an hour at 37° C.

The ceDNA vectors for gene editing as disclosed herein have no packaging constraints imposed by the limiting space within the viral capsid. ceDNA vectors represent a viable eukaryotically-produced alternative to prokaryote-produced plasmid DNA vectors, as opposed to encapsulated AAV genomes. This permits the insertion of control elements, e.g., regulatory switches as disclosed herein, large transgenes, multiple transgenes etc.

In one aspect, a ceDNA vector for gene editing as comprises, in the 5′ to 3′ direction: a first adeno-associated virus (AAV) inverted terminal repeat (ITR), a nucleotide sequence of interest (for example an expression cassette as described herein) and a second AAV ITR. In some embodiments, the first ITR (5′ ITR) and the second ITR (3′ ITR) are asymmetric with respect to each other—that is, they have a different 3D-spatial configuration from one another. As an exemplary embodiment, the first ITR can be a wild-type ITR and the second ITR can be a mutated or modified ITR, or vice versa, where the first ITR can be a mutated or modified ITR and the second ITR a wild-type ITR. In another embodiment, the first ITR and the second ITR are both modified but are different sequences, or have different modifications, or are not identical modified ITRs, and have different 3D spatial configurations. Stated differently, a ceDNA vector for gene editing with asymmetric ITRs have ITRs where any changes in one ITR relative to the WT-ITR are not reflected in the other ITR; or alternatively, where the asymmetric ITRs have a the modified asymmetric ITR pair can have a different sequence and different three-dimensional shape with respect to each other. Exemplary asymmetric ITRs in the ceDNA vector and for use to generate a ceDNA-plasmid are discussed below in the section entitled “asymmetric ITRs”.

In another aspect, a ceDNA vector for gene editing as comprises, in the 5′ to 3′ direction: a first adeno-associated virus (AAV) inverted terminal repeat (ITR), a nucleotide sequence of interest (for example an expression cassette as described herein) and a second AAV ITR, where the first ITR (5′ ITR) and the second ITR (3′ ITR) are symmetric, or substantially symmetrical with respect to each other—that is, a gene editing ceDNA vector can comprise ITR sequences that have a symmetrical three-dimensional spatial organization such that their structure is the same shape in geometrical space, or have the same A, C-C′ and B-B′ loops in 3D space. In such an embodiment, a symmetrical ITR pair, or substantially symmetrical ITR pair can be modified ITRs (e.g., mod-ITRs) that are not wild-type ITRs. A mod-ITR pair can have the same sequence which has one or more modifications from wild-type ITR and are reverse complements (inverted) of each other. In alternative embodiments, a modified ITR pair are substantially symmetrical as defined herein, that is, the modified ITR pair can have a different sequence but have corresponding or the same symmetrical three-dimensional shape. In some embodiments, the symmetrical ITRs, or substantially symmetrical ITRs can be are wild type (WT-ITRs) as described herein. That is, both ITRs have a wild type sequence, but do not necessarily have to be WT-ITRs from the same AAV serotype. That is, in some embodiments, one WT-ITR can be from one AAV serotype, and the other WT-ITR can be from a different AAV serotype. In such an embodiment, a WT-ITR pair are substantially symmetrical as defined herein, that is, they can have one or more conservative nucleotide modification while still retaining the symmetrical three-dimensional spatial organization.

The symmetric ITRs or substantially symmetrical ITRs are discussed in the section below entitled “symmetrical ITR pairs”.

The wild-type or mutated or otherwise modified ITR sequences provided herein represent DNA sequences included in the expression construct (e.g., ceDNA-plasmid, ceDNA Bacmid, ceDNA-baculovirus) for production of the ceDNA vector. Thus, ITR sequences actually contained in the ceDNA vector produced from the ceDNA-plasmid or other expression construct may or may not be identical to the ITR sequences provided herein as a result of naturally occurring changes taking place during the production process (e.g., replication error).

In some embodiments, a ceDNA vector described herein comprising the expression cassette with a transgene which is a gene editing molecule, or a gene editing nucleic acid sequence, can be operatively linked to one or more regulatory sequence(s) that allows or controls expression of the transgene. In one embodiment, the polynucleotide comprises a first ITR sequence and a second ITR sequence, wherein the nucleotide sequence of interest is flanked by the first and second ITR sequences, and the first and second ITR sequences are asymmetrical relative to each other, or symmetrical relative to each other.

In one embodiment in each of these aspects, an expression cassette is located between two ITRs comprised in the following order with one or more of: a promoter operably linked to a transgene, a posttranscriptional regulatory element, and a polyadenylation and termination signal. In one embodiment, the promoter is regulatable—inducible or repressible. The promoter can be any sequence that facilitates the transcription of the transgene. In one embodiment the promoter is a CAG promoter (e.g. SEQ ID NO: 03), or variation thereof. The posttranscriptional regulatory element is a sequence that modulates expression of the transgene, as a non-limiting example, any sequence that creates a tertiary structure that enhances expression of the transgene which is a gene editing molecule, or a gene editing nucleic acid sequence.

In one embodiment, the posttranscriptional regulatory element comprises WPRE (e.g. SEQ ID NO: 08). In one embodiment, the polyadenylation and termination signal comprises BGHpolyA (e.g. SEQ ID NO: 09). Any cis regulatory element known in the art, or combination thereof, can be additionally used e.g., SV40 late polyA signal upstream enhancer sequence (USE), or other posttranscriptional processing elements including, but not limited to, the thymidine kinase gene of herpes simplex virus, or hepatitis B virus (HBV). In one embodiment, the expression cassette length in the 5′ to 3′ direction is greater than the maximum length known to be encapsidated in an AAV virion. In one embodiment, the length is greater than 4.6 kb, or greater than 5 kb, or greater than 6 kb, or greater than 7 kb. Various expression cassettes are exemplified herein.

The expression cassette can comprise more than 4000 nucleotides, 5000 nucleotides, 10,000 nucleotides or 20,000 nucleotides, or 30,000 nucleotides, or 40,000 nucleotides or 50,000 nucleotides, or any range between about 4000-10,000 nucleotides or 10,000-50,000 nucleotides, or more than 50,000 nucleotides. In some embodiments, the expression cassette can comprise a transgene which is a gene editing molecule, or a gene editing nucleic acid sequence in the range of 500 to 50,000 nucleotides in length. In some embodiments, the expression cassette can comprise a transgene which is a gene editing molecule, or a gene editing nucleic acid sequence in the range of 500 to 75,000 nucleotides in length. In some embodiments, the expression cassette can comprise a transgene which is a gene editing molecule, or a gene editing nucleic acid sequence is in the range of 500 to 10,000 nucleotides in length. In some embodiments, the expression cassette can comprise a transgene which is a gene editing molecule, or a gene editing nucleic acid sequence is in the range of 1000 to 10,000 nucleotides in length. In some embodiments, the expression cassette can comprise a transgene which is a gene editing molecule, or a gene editing nucleic acid sequence is in the range of 500 to 5,000 nucleotides in length. The ceDNA vectors do not have the size limitations of encapsidated AAV vectors, thus enable delivery of a large-size expression cassette to provide efficient transgene which is a gene editing molecule, or a gene editing nucleic acid sequence. In some embodiments, the ceDNA vector is devoid of prokaryote-specific methylation.

The expression cassette can also comprise an internal ribosome entry site (IRES) and/or a 2A element. The cis-regulatory elements include, but are not limited to, a promoter, a riboswitch, an insulator, a mir-regulatable element, a post-transcriptional regulatory element, a tissue- and cell type-specific promoter and an enhancer. In some embodiments the ITR can act as the promoter for the transgene. In some embodiments, the ceDNA vector comprises additional components to regulate expression of the transgene, for example, a regulatory switches, which are described herein in the section entitled “Regulatory Switches” for controlling and regulating the expression of the transgene, and can include if desired, a regulatory switch which is a kill switch to enable controlled cell death of a cell comprising a ceDNA vector.

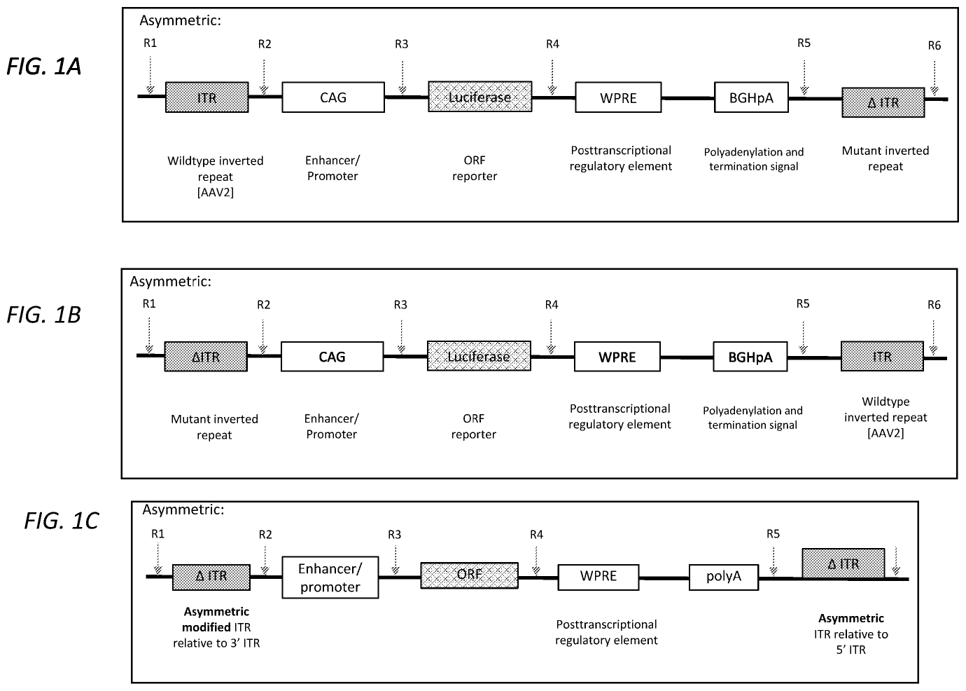

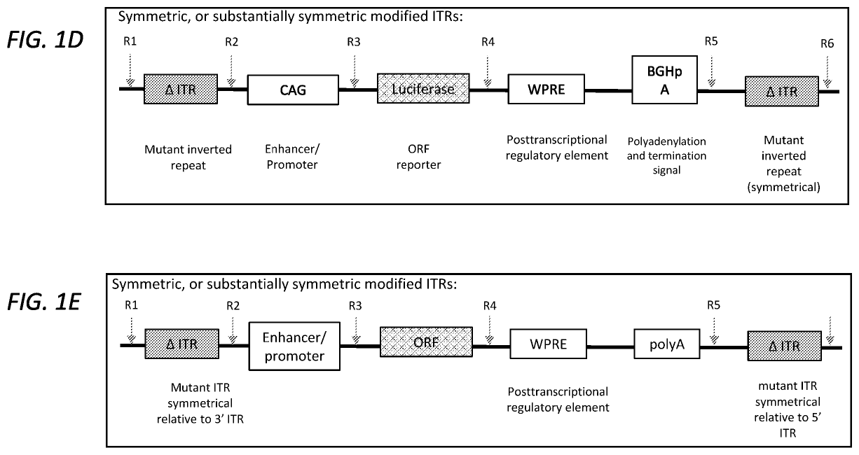

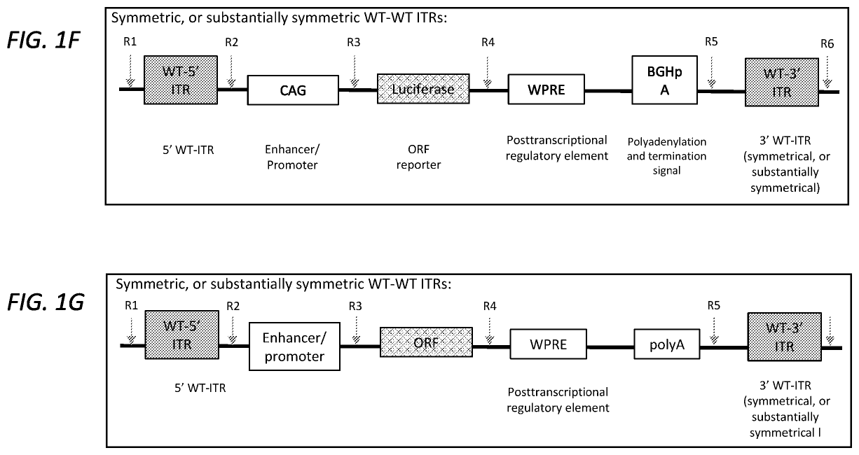

FIG. 1A-1E show schematics of nonlimiting, exemplary ceDNA vectors, or the corresponding sequence of ceDNA plasmids. ceDNA vectors are capsid-free and can be obtained from a plasmid encoding in this order: a first ITR, expressible transgene cassette and a second ITR, where at least one of the first and/or second ITR sequence is mutated with respect to the corresponding wild type AAV2 ITR sequence. The cassette preferably includes one or more of, in this order: an enhancer/promoter, an ORF reporter (transgene), a post-transcription regulatory element (e.g., WPRE), and a polyadenylation and termination signal (e.g., BGH polyA).

The expression cassette can comprise any transgene which is a gene editing molecule, or a gene editing nucleic acid sequence. The gene editing ceDNA vector edit any gene of interest in the subject, which includes but are not limited to, nucleic acids encoding polypeptides, or non-coding nucleic acids (e.g., RNAi, miRs etc.), as well as exogenous genes and nucleotide sequences, including virus sequences in a subjects' genome, e.g., HIV virus sequences and the like. Preferably the gene editing ceDNA vector disclosed herein is used for therapeutic purposes (e.g., for medical, diagnostic, or veterinary uses) or immunogenic polypeptides. In certain embodiments, the gene editing ceDNA vector can edit any gene of interest in the subject, which includes one or more polypeptides, peptides, ribozymes, peptide nucleic acids, siRNAs, RNAis, antisense oligonucleotides, antisense polynucleotides, antibodies, antigen binding fragments, or any combination thereof.

ceDNA expression cassette can include, for example, an expressible exogenous sequence (e.g., open reading frame) that encodes a protein that is either absent, inactive, or insufficient activity in the recipient subject or a gene that encodes a protein having a desired biological or a therapeutic effect. The exogenous sequence such as a donor sequence can encode a gene product that can function to correct the expression of a defective gene or transcript. The expression cassette can also encode corrective DNA strands, encode polypeptides, sense or antisense oligonucleotides, or RNAs (coding or non-coding; e.g., siRNAs, shRNAs, micro-RNAs, and their antisense counterparts (e.g., antagoMiR)). Expression cassettes can include an exogenous sequence that encodes a reporter protein to be used for experimental or diagnostic purposes, such as β-lactamase, β-galactosidase (LacZ), alkaline phosphatase, thymidine kinase, green fluorescent protein (GFP), chloramphenicol acetyltransferase (CAT), luciferase, and others well known in the art.

In principle, the expression cassette can include any gene that encodes a protein, polypeptide or RNA that is either reduced or absent due to a mutation or which conveys a therapeutic benefit when overexpressed is considered to be within the scope of the disclosure. The ceDNA vector may comprise a template or donor nucleotide sequence used as a correcting DNA strand to be inserted after a double-strand break (or nick) provided by a nuclease. The ceDNA vector may include a template nucleotide sequence used as a correcting DNA strand to be inserted after a double-strand break (or nick) provided by a guided RNA nuclease, meganuclease, or zinc finger nuclease. Preferably, non-inserted bacterial DNA is not present and preferably no bacterial DNA is present in the ceDNA compositions provided herein. In some instances, the protein can change a codon without a nick.

Sequences provided in the expression cassette, expression construct, or donor sequence of a ceDNA vector described herein can be codon optimized for the host cell. As used herein, the term “codon optimized” or “codon optimization” refers to the process of modifying a nucleic acid sequence for enhanced expression in the cells of the vertebrate of interest, e.g., mouse or human, by replacing at least one, more than one, or a significant number of codons of the native sequence (e.g., a prokaryotic sequence) with codons that are more frequently or most frequently used in the genes of that vertebrate. Various species exhibit particular bias for certain codons of a particular amino acid. Typically, codon optimization does not alter the amino acid sequence of the original translated protein. Optimized codons can be determined using e.g., Aptagen's Gene Forge® codon optimization and custom gene synthesis platform (Aptagen, Inc., 2190 Fox Mill Rd. Suite 300, Herndon, Va. 20171) or another publicly available database.

Many organisms display a bias for use of particular codons to code for insertion of a particular amino acid in a growing peptide chain. Codon preference or codon bias, differences in codon usage between organisms, is afforded by degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

Given the large number of gene sequences available for a wide variety of animal, plant and microbial species, it is possible to calculate the relative frequencies of codon usage (Nakamura, Y., et al. “Codon usage tabulated from the international DNA sequence databases: status for the year 2000” Nucl. Acids Res. 28:292 (2000)).

In some embodiments, the gene editing gene (e.g., donor sequences) or guide RNA targets a therapeutic gene. In some embodiments, the guide RNA targets an antibody, or antibody fragment, or antigen-binding fragment thereof, e.g., a neutralizing antibody or antibody fragment and the like.

In particular, the gene editing gene (e.g., donor sequences) or guide RNA targets one or more therapeutic agent(s), including, but not limited to, for example, protein(s), polypeptide(s), peptide(s), enzyme(s), antibodies, antigen binding fragments, as well as variants, and/or active fragments thereof, for use in the treatment, prophylaxis, and/or amelioration of one or more symptoms of a disease, dysfunction, injury, and/or disorder. Exemplary genes for targeting with the guide RNA are described herein in the section entitled “Method of Treatment”.

There are many structural features of ceDNA vectors that differ from plasmid-based expression vectors. ceDNA vectors may possess one or more of the following features: the lack of original (i.e. not inserted) bacterial DNA, the lack of a prokaryotic origin of replication, being self-containing, i.e., they do not require any sequences other than the two ITRs, including the Rep binding and terminal resolution sites (RBS and TRS), and an exogenous sequence between the ITRs, the presence of ITR sequences that form hairpins, of the eukaryotic origin (i.e., they are produced in eukaryotic cells), and the absence of bacterial-type DNA methylation or indeed any other methylation considered abnormal by a mammalian host. In general, it is preferred for the present vectors not to contain any prokaryotic DNA but it is contemplated that some prokaryotic DNA may be inserted as an exogenous sequence, as a nonlimiting example in a promoter or enhancer region. Another important feature distinguishing ceDNA vectors from plasmid expression vectors is that ceDNA vectors are single-strand linear DNA having closed ends, while plasmids are always double-stranded DNA.

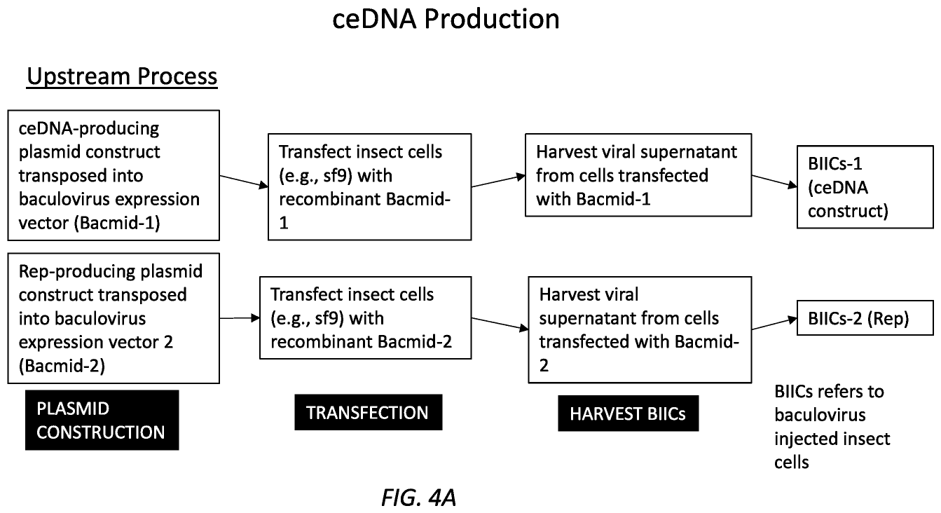

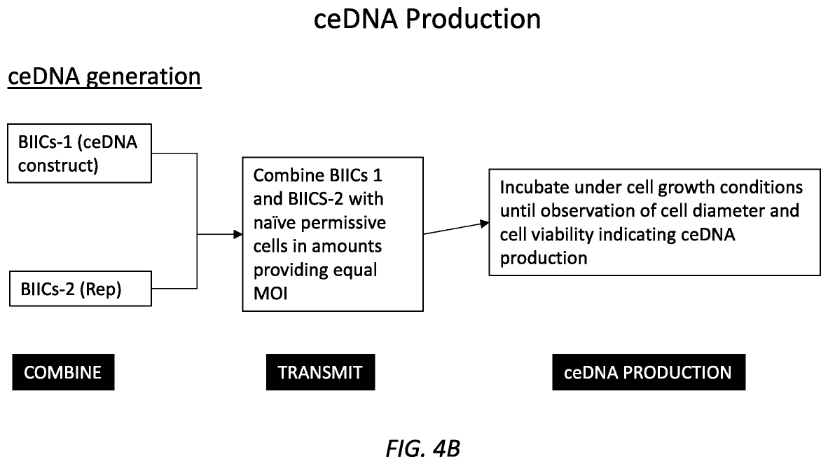

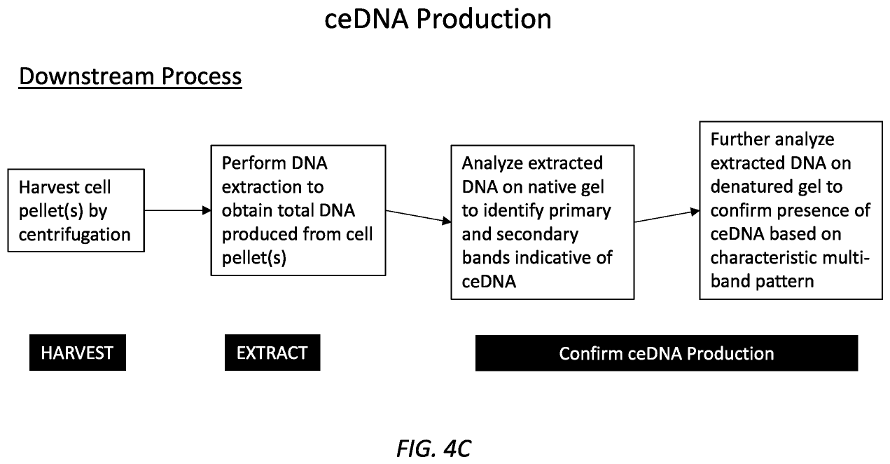

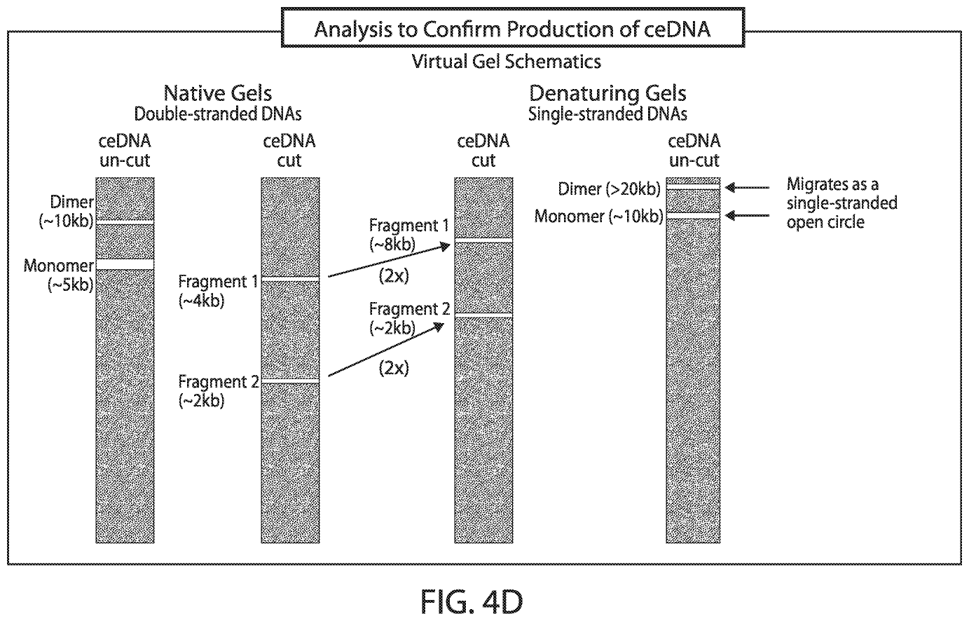

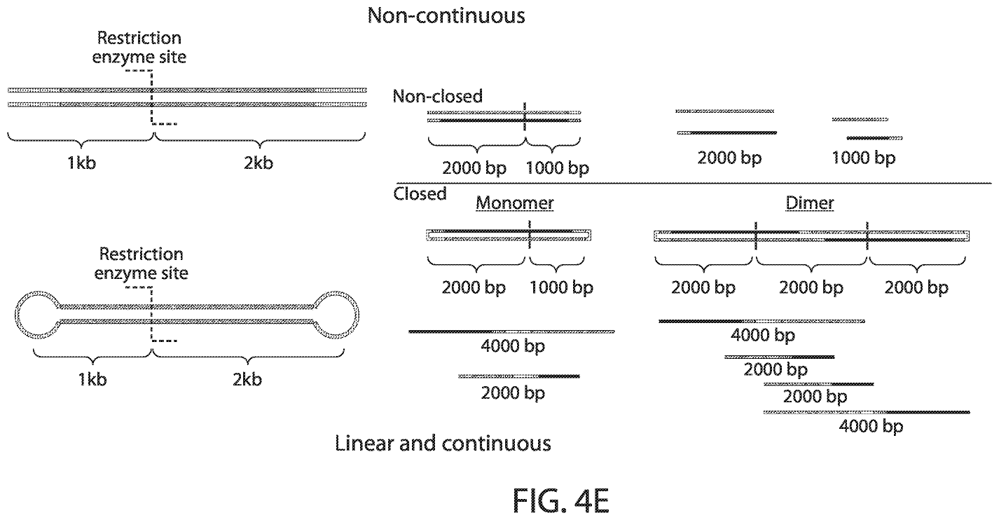

ceDNA vectors for gene editing produced by the methods provided herein preferably have a linear and continuous structure rather than a non-continuous structure, as determined by restriction enzyme digestion assay (FIG. 4D). The linear and continuous structure is believed to be more stable from attack by cellular endonucleases, as well as less likely to be recombined and cause mutagenesis. Thus, a gene editing ceDNA vector in the linear and continuous structure is a preferred embodiment. The continuous, linear, single strand intramolecular duplex ceDNA vector can have covalently bound terminal ends, without sequences encoding AAV capsid proteins. These gene editing ceDNA vectors are structurally distinct from plasmids (including ceDNA plasmids described herein), which are circular duplex nucleic acid molecules of bacterial origin. The complimentary strands of plasmids may be separated following denaturation to produce two nucleic acid molecules, whereas in contrast, ceDNA vectors, while having complimentary strands, are a single DNA molecule and therefore even if denatured, remain a single molecule. In some embodiments, ceDNA vectors as described herein can be produced without DNA base methylation of prokaryotic type, unlike plasmids. Therefore, the ceDNA vectors and ceDNA-plasmids are different both in term of structure (in particular, linear versus circular) and also in view of the methods used for producing and purifying these different objects (see below), and also in view of their DNA methylation which is of prokaryotic type for ceDNA-plasmids and of eukaryotic type for the ceDNA vector.

There are several advantages of using a ceDNA vector as described herein for gene editing over plasmid-based expression vectors, such advantages include, but are not limited to: 1) plasmids contain bacterial DNA sequences and are subjected to prokaryotic-specific methylation, e.g., 6-methyl adenosine and 5-methyl cytosine methylation, whereas capsid-free AAV vector sequences are of eukaryotic origin and do not undergo prokaryotic-specific methylation; as a result, capsid-free AAV vectors are less likely to induce inflammatory and immune responses compared to plasmids; 2) while plasmids require the presence of a resistance gene during the production process, ceDNA vectors do not; 3) while a circular plasmid is not delivered to the nucleus upon introduction into a cell and requires overloading to bypass degradation by cellular nucleases, ceDNA vectors contain viral cis-elements, i.e., ITRs, that confer resistance to nucleases and can be designed to be targeted and delivered to the nucleus. It is hypothesized that the minimal defining elements indispensable for ITR function are a Rep-binding site (RBS; 5′-GCGCGCTCGCTCGCTC-3′ (SEQ ID NO: 531) for AAV2) and a terminal resolution site (TRS; 5′-AGTTGG-3′ (SEQ ID NO: 48) for AAV2) plus a variable palindromic sequence allowing for hairpin formation; and 4) ceDNA vectors do not have the over-representation of CpG dinucleotides often found in prokaryote-derived plasmids that reportedly binds a member of the Toll-like family of receptors, eliciting a T cell-mediated immune response. In contrast, transductions with capsid-free AAV vectors disclosed herein can efficiently target cell and tissue-types that are difficult to transduce with conventional AAV virions using various delivery reagent.

### III. Knock-In of a Desired Nucleic Acid Sequence

The gene editing ceDNA vectors, methods and compositions described herein can be used to introduce a new nucleic acid sequence, correct a mutation of a genomic sequence or introduce a mutation into a target gene sequence in a host cell. Such methods can be referred to as “DNA knock-in systems.” The DNA knock-in system, as described herein, allows donor sequences to be inserted at any desired target site with high efficiency, making it feasible for many uses such as creation of transgenic animals expressing exogenous genes, preparing cell culture models of disease, preparing screening assay systems, modifying gene expression of engineered tissue constructs, modifying (e.g., mutating) a genomic locus, and gene editing, for example by adding an exogenous non-coding sequence (such as sequence tags or regulatory elements) into the genome. The cells and animals produced using methods provided herein can find various applications, for example as cellular therapeutics, as disease models, as research tools, and as humanized animals useful for various purposes.

The DNA knock-in systems of the present disclosure also allow for gene editing techniques using large donor sequences (<5 kb) to be inserted at any desired target site in a genome, thus providing gene editing of larger genes than current techniques. In some embodiments, large homology arms, for example 50 base pairs to two thousand base pairs, are included providing gene editing with excellent efficiency (higher on-target) and excellent specificity (lower off-target), and in some embodiments, HDR without the use of nucleases.

The DNA knock-in systems of the present disclosure also provide several advantages with respect to the administration of donor sequences for gene editing. First, administering ceDNA vectors as described herein within delivery particles of the present disclosure is not precluded by baseline immunity and therefore can be administered to any and potentially all patients with a particular disorder. Second, administering particles of the present disclosure does not create an adaptive immune response to the delivered therapeutic like that typically raised against viral vector-based delivery systems and therefore embodiments can be re-dosed as needed for clinical effect. Administration of one or more ceDNA vectors in accordance with the present disclosure, such as in vivo delivery, is repeatable and robust.

In certain embodiments, gene editing with ceDNA vectors of the present disclosure can be monitored with appropriate biomarkers from treated patients to assess the efficiency of the gene correction, and repeat administrations of the therapeutic product can be made until the appropriate level of gene editing has been achieved.

In another aspect, there is provided a method of generating a genetically modified animal by using the gene knock-in system described herein with ceDNA vectors in accordance with the present disclosure. These methods are described further below.

In certain embodiments, the present disclosure relates to methods of using a ceDNA vector for inserting a donor sequence at a predetermined insertion site on a chromosome of a host cell, such as a eukaryotic or prokaryotic cell.

### IV. Gene Editing System Components—General

In further embodiments, such as those including an RNA guided nuclease, the components required for gene editing may include a nuclease, a guide RNA (if Cas9 or the like is utilized), a donor sequence and one or more homology arms included within a single ceDNA vector of the present disclosure. Such embodiments increase the efficiency of gene editing compared to approaches that require distinct or various particles to deliver the gene editing components.

In further embodiments, a nuclease can be inactivated/diminished after gene editing, reducing or eliminating off-target editing, if any, that would otherwise occur with the persistence of an added nuclease within cells.

In another aspect, the present disclosure relates to kits including one or more ceDNA vectors for use in any one of the methods described herein.

The methods and compositions described herein also provide for gene editing systems comprising a cellular switch, for example, as described by Oakes et al. Nat. Biotechnol. 34:646-651 (2016), the contents of which are herein incorporated by reference in their entirety.

It is also specifically contemplated herein that the methods and compositions described herein can be performed in a high-throughput manner using methods known in the art (see e.g., Shalem et al. Nat Rev Genet 16:299-311 (2015); Shalem et al. Science 343:84-88 (2014); the contents of each of which are incorporated herein by reference in their entirety.

### V. ITRs

As disclosed herein, ceDNA vectors contain a gene editing nucleic acid sequence positioned between two inverted terminal repeat (ITR) sequences, where the ITR sequences can be an asymmetrical ITR pair or a symmetrical- or substantially symmetrical ITR pair, as these terms are defined herein. A ceDNA vector for gene editing disclosed herein can comprise ITR sequences that are selected from any of: (i) at least one WT ITR and at least one modified AAV inverted terminal repeat (mod-ITR) (e.g., asymmetric modified ITRs); (ii) two modified ITRs where the mod-ITR pair have a different three-dimensional spatial organization with respect to each other (e.g., asymmetric modified ITRs), or (iii) symmetrical or substantially symmetrical WT-WT ITR pair, where each WT-ITR has the same three-dimensional spatial organization, or (iv) symmetrical or substantially symmetrical modified ITR pair, where each mod-ITR has the same three-dimensional spatial organization, where the methods of the present disclosure may further include a delivery system, such as but not limited to a liposome nanoparticle delivery system.

A. Symmetrical ITR Pairs

In some embodiments, the ITR sequence can be from viruses of the Parvoviridae family, which includes two subfamilies Parvovirinae, which infect vertebrates, and Densovirinae, which infect insects. The subfamily Parvovirinae (referred to as the parvoviruses) includes the genus Dependovirus, the members of which, under most conditions, require coinfection with a helper virus such as adenovirus or herpes virus for productive infection. The genus Dependovirus includes adeno-associated virus (AAV), which normally infects humans (e.g., serotypes 2, 3A, 3B, 5, and 6) or primates (e.g., serotypes 1 and 4), and related viruses that infect other warm-blooded animals (e.g., bovine, canine, equine, and ovine adeno-associated viruses). The parvoviruses and other members of the Parvoviridae family are generally described in Kenneth I. Berns, “Parvoviridae: The Viruses and Their Replication,” Chapter 69 in FIELDS VIROLOGY (3d Ed. 1996).

While ITRs exemplified in the specification and Examples herein are AAV2 WT-ITRs, one of ordinary skill in the art is aware that one can as stated above use ITRs from any known parvovirus, for example a dependovirus such as AAV (e.g., AAV1, AAV2, AAV3, AAV4, AAVS, AAV 5, AAV7, AAV8, AAV9, AAV10, AAV 11, AAV12, AAVrh8, AAVrh10, AAV-DJ, and AAV-DJ8 genome. E.g., NCBI: NC 002077; NC 001401; NC001729; NC001829; NC006152; NC 006260; NC 006261), chimeric ITRs, or ITRs from any synthetic AAV. In some embodiments, the AAV can infect warm-blooded animals, e.g., avian (AAAV), bovine (BAAV), canine, equine, and ovine adeno-associated viruses. In some embodiments the ITR is from B19 parvovirus (GenBank Accession No: NC 000883), Minute Virus from Mouse (MVM) (GenBank Accession No. NC 001510); goose parvovirus (GenBank Accession No. NC 001701); snake parvovirus 1 (GenBank Accession No. NC 006148). In some embodiments, the 5′ WT-ITR can be from one serotype and the 3′ WT-ITR from a different serotype, as discussed herein.

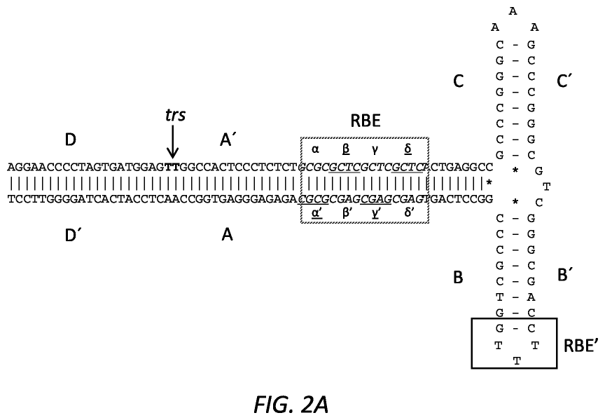

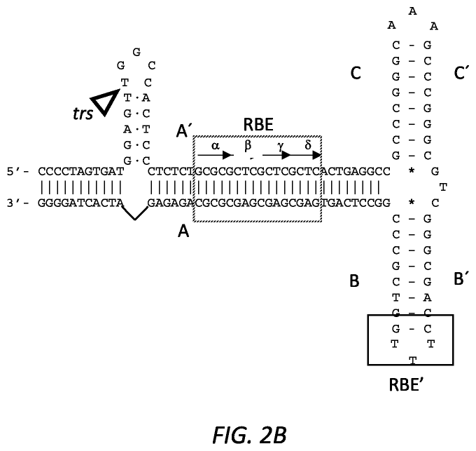

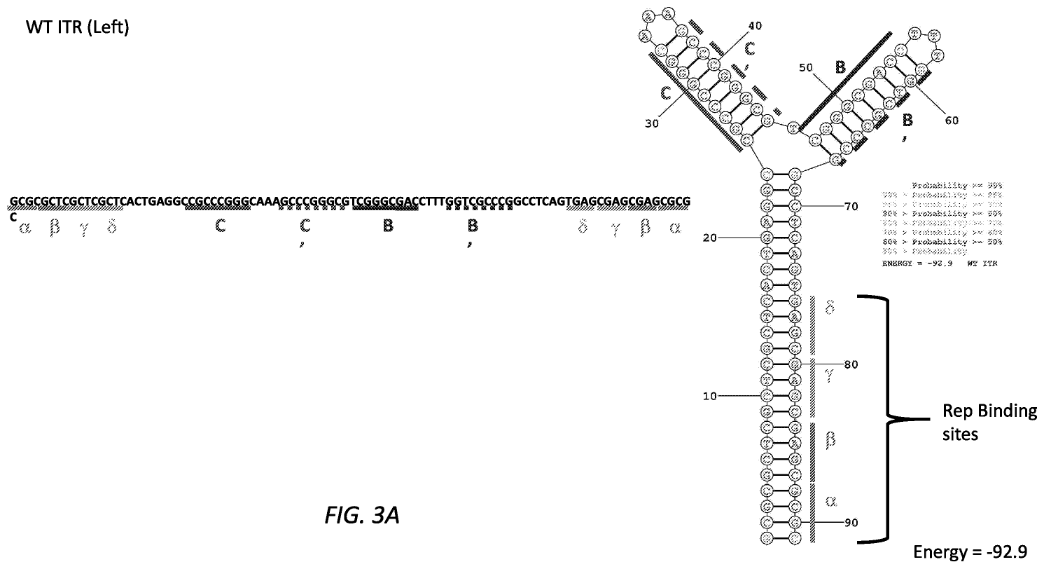

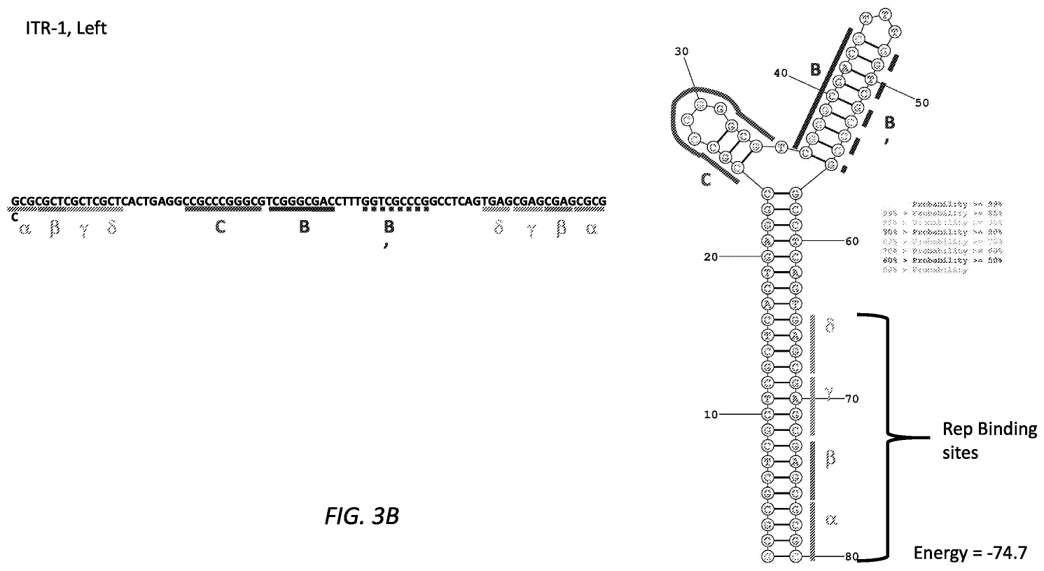

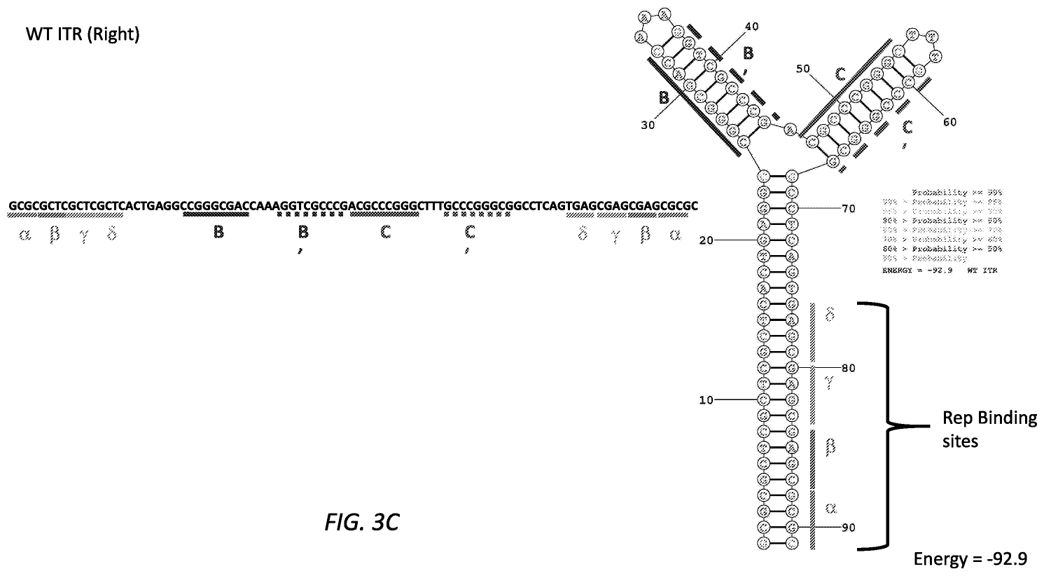

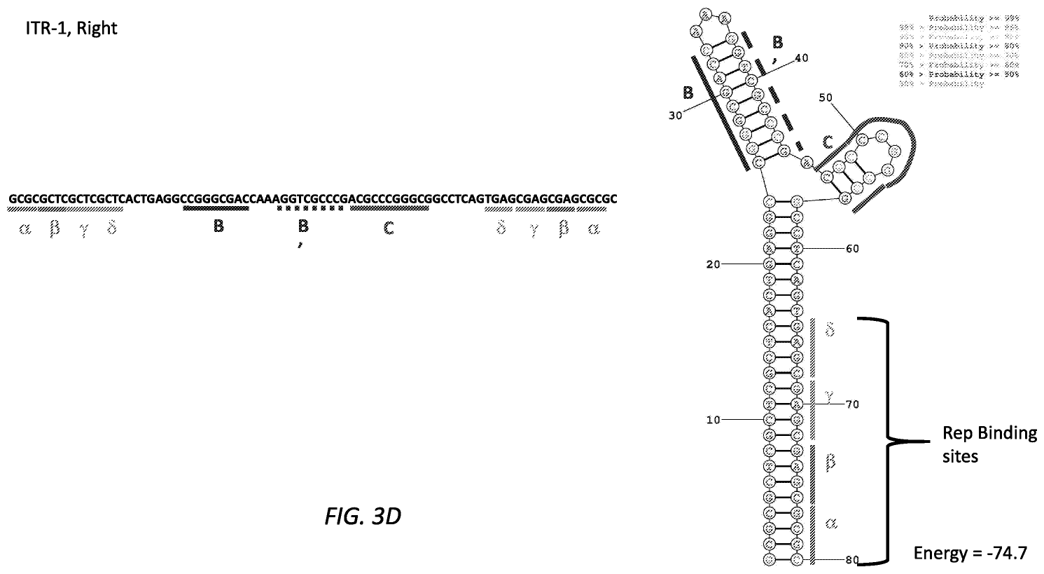

An ordinarily skilled artisan is aware that ITR sequences have a common structure of a double-stranded Holliday junction, which typically is a T-shaped or Y-shaped hairpin structure (see e.g., FIG. 2A and FIG. 3A), where each WT-ITR is formed by two palindromic arms or loops (B-B′ and C-C′) embedded in a larger palindromic arm (A-A′), and a single stranded D sequence, (where the order of these palindromic sequences defines the flip or flop orientation of the ITR). See, for example, structural analysis and sequence comparison of ITRs from different AAV serotypes (AAV1-AAV6) and described in Grimm et al., J. Virology, 2006; 80(1); 426-439; Yan et al., J. Virology, 2005; 364-379; Duan et al., Virology 1999; 261; 8-14. One of ordinary skill in the art can readily determine WT-ITR sequences from any AAV serotype for use in a ceDNA vector or ceDNA-plasmid based on the exemplary AAV2 ITR sequences provided herein. See, for example, the sequence comparison of ITRs from different AAV serotypes (AAV1-AAV6, and avian AAV (AAAV) and bovine AAV (BAAV)) described in Grimm et al., J. Virology, 2006; 80(1); 426-439; that show the % identity of the left ITR of AAV2 to the left ITR from other serotypes: AAV-1 (84%), AAV-3 (86%), AAV-4 (79%), AAV-5 (58%), AAV-6 (left ITR) (100%) and AAV-6 (right ITR) (82%).

As discussed herein, in some embodiments a ceDNA vector for gene editing can comprise symmetric ITR sequences (e.g., a symmetrical ITR pair), where the 5′ ITR and the 3′ ITR can have the same symmetrical three-dimensional organization with respect to each other, (i.e., symmetrical or substantially symmetrical). That is—a ceDNA vector for gene editing comprises ITR sequences that have a symmetrical three-dimensional spatial organization such that their structure is the same shape in geometrical space, or have the same A, C-C′ and B-B′ loops in 3D space (i.e., they are the same or are mirror images with respect to each other). In such an embodiment, a symmetrical ITR pair, or substantially symmetrical ITR pair can be modified ITRs (e.g., mod-ITRs) that are not wild-type ITRs. A mod-ITR pair can have the same sequence which has one or more modifications from wild-type ITR and are reverse complements (inverted) of each other. In alternative embodiments, a modified ITR pair are substantially symmetrical as defined herein, that is, the modified ITR pair can have a different sequence but have corresponding or the same symmetrical three-dimensional shape.

(i) Wildtype ITRs

In some embodiments, the symmetrical ITRs, or substantially symmetrical ITRs are wild type (WT-ITRs) as described herein. That is, both ITRs have a wild type sequence, but do not necessarily have to be WT-ITRs from the same AAV serotype. That is, in some embodiments, one WT-ITR can be from one AAV serotype, and the other WT-ITR can be from a different AAV serotype. In such an embodiment, a WT-ITR pair are substantially symmetrical as defined herein, that is, they can have one or more conservative nucleotide modification while still retaining the symmetrical three-dimensional spatial organization.

Accordingly, as disclosed herein, ceDNA vectors for gene editing contain a gene editing sequence positioned between two flanking wild-type inverted terminal repeat (WT-ITR) sequences, that are either the reverse complement (inverted) of each other, or alternatively, are substantially symmetrical relative to each other—that is a WT-ITR pair have symmetrical three-dimensional spatial organization. In some embodiments, a wild-type ITR sequence (e.g. AAV WT-ITR) comprises a functional Rep binding site (RBS; e.g. 5′-GCGCGCTCGCTCGCTC-3′ for AAV2, SEQ ID NO: 531) and a functional terminal resolution site (TRS; e.g. 5′-AGTT-3′, SEQ ID NO: 46).

In one aspect, ceDNA vectors for gene editing are obtainable from a vector polynucleotide that encodes a heterologous nucleic acid operatively positioned between two WT inverted terminal repeat sequences (WT-ITRs) (e.g. AAV WT-ITRs). That is, both ITRs have a wild type sequence, but do not necessarily have to be WT-ITRs from the same AAV serotype. That is, in some embodiments, one WT-ITR can be from one AAV serotype, and the other WT-ITR can be from a different AAV serotype. In such an embodiment, the WT-ITR pair are substantially symmetrical as defined herein, that is, they can have one or more conservative nucleotide modification while still retaining the symmetrical three-dimensional spatial organization. In some embodiments, the 5′ WT-ITR is from one AAV serotype, and the 3′ WT-ITR is from the same or a different AAV serotype. In some embodiments, the 5′ WT-ITR and the 3′WT-ITR are mirror images of each other, that is they are symmetrical. In some embodiments, the 5′ WT-ITR and the 3′ WT-ITR are from the same AAV serotype.

WT ITRs are well known. In one embodiment the two ITRs are from the same AAV2 serotype. In certain embodiments one can use WT from other serotypes. There are a number of serotypes that are homologous, e.g. AAV2, AAV4, AAV6, AAV8. In one embodiment, closely homologous ITRs (e.g. ITRs with a similar loop structure) can be used. In another embodiment, one can use AAV WT ITRs that are more diverse, e.g., AAV2 and AAVS, and still another embodiment, one can use an ITR that is substantially WT—that is, it has the basic loop structure of the WT but some conservative nucleotide changes that do not alter or affect the properties. When using WT-ITRs from the same viral serotype, one or more regulatory sequences may further be used. In certain embodiments, the regulatory sequence is a regulatory switch that permits modulation of the activity of the ceDNA.

In some embodiments, one aspect of the technology described herein relates to a non-viral capsid-free DNA vector with covalently-closed ends (ceDNA vector), wherein the ceDNA vector comprises at least one heterologous nucleotide sequence, operably positioned between two wild-type inverted terminal repeat sequences (WT-ITRs), wherein the WT-ITRs can be from the same serotype, different serotypes or substantially symmetrical with respect to each other (i.e., have the symmetrical three-dimensional spatial organization such that their structure is the same shape in geometrical space, or have the same A, C-C′ and B-B′ loops in 3D space). In some embodiments, the symmetric WT-ITRs comprises a functional terminal resolution site and a Rep binding site. In some embodiments, the heterologous nucleic acid sequence encodes a transgene, and wherein the vector is not in a viral capsid.

In some embodiments, the WT-ITRs are the same but the reverse complement of each other. For example, the sequence AACG in the 5′ ITR may be CGTT (i.e., the reverse complement) in the 3′ ITR at the corresponding site. In one example, the 5′ WT-ITR sense strand comprises the sequence of ATCGATCG and the corresponding 3′ WT-ITR sense strand comprises CGATCGAT (i.e., the reverse complement of ATCGATCG). In some embodiments, the WT-ITRs ceDNA further comprises a terminal resolution site and a replication protein binding site (RPS) (sometimes referred to as a replicative protein binding site), e.g. a Rep binding site.

Exemplary WT-ITR sequences for use in the ceDNA vectors comprising WT-ITRs are shown in Table 2 herein, which shows pairs of WT-ITRs (5′ WT-ITR and the 3′ WT-ITR).

As an exemplary example, the present disclosure provides a closed-ended DNA vector comprising a promoter operably linked to a transgene (e.g., gene editing sequence), with or without the regulatory switch, where the ceDNA is devoid of capsid proteins and is: (a) produced from a ceDNA-plasmid (e.g., see FIGS. 1F-1G) that encodes WT-ITRs, where each WT-ITR has the same number of intramolecularly duplexed base pairs in its hairpin secondary configuration (preferably excluding deletion of any AAA or TTT terminal loop in this configuration compared to these reference sequences), and (b) is identified as ceDNA using the assay for the identification of ceDNA by agarose gel electrophoresis under native gel and denaturing conditions in Example 1.

In some embodiments, the flanking WT-ITRs are substantially symmetrical to each other. In this embodiment the 5′ WT-ITR can be from one serotype of AAV, and the 3′ WT-ITR from a different serotype of AAV, such that the WT-ITRs are not identical reverse complements. For example, the 5′ WT-ITR can be from AAV2, and the 3′ WT-ITR from a different serotype (e.g. AAV1, 3, 4, 5, 6, 7, 8, 9, 10, 11, and 12. In some embodiments, WT-ITRs can be selected from two different parvoviruses selected from any to of: AAV1, AAV2, AAV3, AAV4, AAV5, AAV6, AAV7, AAV8, AAV9, AAV10, AAV11, AAV12, AAV13, snake parvovirus (e.g., royal python parvovirus), bovine parvovirus, goat parvovirus, avian parvovirus, canine parvovirus, equine parvovirus, shrimp parvovirus, porcine parvovirus, or insect AAV. In some embodiments, such a combination of WT ITRs is the combination of WT-ITRs from AAV2 and AAV6. In one embodiment, the substantially symmetrical WT-ITRs are when one is inverted relative to the other ITR at least 90% identical, at least 95% identical, at least 96% . . . 97% . . . 98% . . . 99% . . . . 99.5% and all points in between, and has the same symmetrical three-dimensional spatial organization. In some embodiments, a WT-ITR pair are substantially symmetrical as they have symmetrical three-dimensional spatial organization, e.g., have the same 3D organization of the A, C-C′. B-B′ and D arms. In one embodiment, a substantially symmetrical WT-ITR pair are inverted relative to the other, and are at least 95% identical, at least 96% . . . 97% . . . 98% . . . 99% . . . . 99.5% and all points in between, to each other, and one WT-ITR retains the Rep-binding site (RBS) of 5′-GCGCGCTCGCTCGCTC-3′ (SEQ ID NO: 531) and a terminal resolution site (trs). In some embodiments, a substantially symmetrical WT-ITR pair are inverted relative to each other, and are at least 95% identical, at least 96% . . . 97% . . . 98% . . . 99% . . . . 99.5% and all points in between, to each other, and one WT-ITR retains the Rep-binding site (RBS) of 5′-GCGCGCTCGCTCGCTC-3′ (SEQ ID NO: 531) and a terminal resolution site (trs) and in addition to a variable palindromic sequence allowing for hairpin secondary structure formation. Homology can be determined by standard means well known in the art such as BLAST (Basic Local Alignment Search Tool), BLASTN at default setting.

In some embodiments, the structural element of the ITR can be any structural element that is involved in the functional interaction of the ITR with a large Rep protein (e.g., Rep 78 or Rep 68). In certain embodiments, the structural element provides selectivity to the interaction of an ITR with a large Rep protein, i.e., determines at least in part which Rep protein functionally interacts with the ITR. In other embodiments, the structural element physically interacts with a large Rep protein when the Rep protein is bound to the ITR. Each structural element can be, e.g., a secondary structure of the ITR, a nucleotide sequence of the ITR, a spacing between two or more elements, or a combination of any of the above. In one embodiment, the structural elements are selected from the group consisting of an A and an A′ arm, a B and a B′ arm, a C and a C′ arm, a D arm, a Rep binding site (RBE) and an RBE′ (i.e., complementary RBE sequence), and a terminal resolution sire (trs).

By way of example only, Table 1 indicates exemplary combinations of WT-ITRs.

Table 1: Exemplary combinations of WT-ITRs from the same serotype or different serotypes, or different parvoviruses. The order shown is not indicative of the ITR position, for example, “AAV1, AAV2” demonstrates that the ceDNA can comprise a WT-AAV1 ITR in the 5′ position, and a WT-AAV2 ITR in the 3′ position, or vice versa, a WT-AAV2 ITR the 5′ position, and a WT-AAV1 ITR in the 3′ position. Abbreviations: AAV serotype 1 (AAV1), AAV serotype 2 (AAV2), AAV serotype 3 (AAV3), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), AAV serotype 11 (AAV11), or AAV serotype 12 (AAV12); AAVrh8, AAVrh10, AAV-DJ, and AAV-DJ8 genome (E.g., NCBI: NC 002077; NC 001401; NC001729; NC001829; NC006152; NC 006260; NC 006261), ITRs from warm-blooded animals (avian AAV (AAAV), bovine AAV (BAAV), canine, equine, and ovine AAV), ITRs from B19 parvovirus (GenBank Accession No: NC 000883), Minute Virus from Mouse (MVM) (GenBank Accession No. NC 001510); Goose: goose parvovirus (GenBank Accession No. NC 001701); snake: snake parvovirus 1 (GenBank Accession No. NC 006148).

TABLE 1

| | | | | |
|---|---|---|---|---|
| AAV1, AAV1 | AAV2, AAV2 | AAV3, AAV3 | AAV4, AAV4 | AAV5, AAV5 |
| AAV1, AAV2 | AAV2, AAV3 | AAV3, AAV4 | AAV4, AAV5 | AAV5, AAV6 |
| AAV1, AAV3 | AAV2, AAV4 | AAV3, AAV5 | AAV4, AAV6 | AAV5, AAV7 |
| AAV1, AAV4 | AAV2, AAV5 | AAV3, AAV6 | AAV4, AAV7 | AAV5, AAV8 |
| AAV1, AAV5 | AAV2, AAV6 | AAV3, AAV7 | AAV4, AAV8 | AAV5, AAV9 |
| AAV1, AAV6 | AAV2, AAV7 | AAV3, AAV8 | AAV4, AAV9 | AAV5, AAV10 |
| AAV1, AAV7 | AAV2, AAV8 | AAV3, AAV9 | AAV4, AAV10 | AAV5, AAV11 |
| AAV1, AAV8 | AAV2, AAV9 | AAV3, AAV10 | AAV4, AAV11 | AAV5, AAV12 |
| AAV1, AAV9 | AAV2, AAV10 | AAV3, AAV11 | AAV4, AAV12 | AAV5, AAVRH8 |
| AAV1, AAV10 | AAV2, AAV11 | AAV3, AAV12 | AAV4, AAVRH8 | AAV5, AAVRH10 |
| AAV1, AAV11 | AAV2, AAV12 | AAV3, AAVRH8 | AAV4, AAVRH10 | AAV5, AAV13 |
| AAV1, AAV12 | AAV2, AAVRH8 | AAV3, AAVRH10 | AAV4, AAV13 | AAV5, AAVDJ |
| AAV1, AAVRH8 | AAV2, AAVRH10 | AAV3, AAV13 | AAV4, AAVDJ | AAV5, AAVDJ8 |
| AAV1, AAVRH10 | AAV2, AAV13 | AAV3, AAVDJ | AAV4, AAVDJ8 | AAV5, AVIAN |
| AAV1, AAV13 | AAV2, AAVDJ | AAV3, AAVDJ8 | AAV4, AVIAN | AAV5, BOVINE |
| AAV1, AAVDJ | AAV2, AAVDJ8 | AAV3, AVIAN | AAV4, BOVINE | AAV5, CANINE |
| AAV1, AAVDJ8 | AAV2, AVIAN | AAV3, BOVINE | AAV4, CANINE | AAV5, EQUINE |
| AAV1, AVIAN | AAV2, BOVINE | AAV3, CANINE | AAV4, EQUINE | AAV5, GOAT |
| AAV1, BOVINE | AAV2, CANINE | AAV3, EQUINE | AAV4, GOAT | AAV5, SHRIMP |
| AAV1, CANINE | AAV2, EQUINE | AAV3, GOAT | AAV4, SHRIMP | AAV5, PORCINE |
| AAV1, EQUINE | AAV2, GOAT | AAV3, SHRIMP | AAV4, PORCINE | AAV5, INSECT |
| AAV1, GOAT | AAV2, SHRIMP | AAV3, PORCINE | AAV4, INSECT | AAV5, OVINE |
| AAV1, SHRIMP | AAV2, PORCINE | AAV3, INSECT | AAV4, OVINE | AAV5, B19 |
| AAV1, PORCINE | AAV2, INSECT | AAV3, OVINE | AAV4, B19 | AAV5, MVM |
| AAV1, INSECT | AAV2, OVINE | AAV3, B19 | AAV4, MVM | AAV5, GOOSE |
| AAV1, OVINE | AAV2, B19 | AAV3, MVM | AAV4, GOOSE | AAV5, SNAKE |
| AAV1, B19 | AAV2, MVM | AAV3, GOOSE | AAV4, SNAKE | |
| AAV1, MVM | AAV2, GOOSE | AAV3, SNAKE | | |
| AAV1, GOOSE | AAV2, SNAKE | | | |
| AAV1, SNAKE | | | | |
| AAV6, AAV6 | AAV7, AAV7 | AAV8, AAV8 | AAV9, AAV9 | AAV10, AAV10 |
| AAV6, AAV7 | AAV7, AAV8 | AAV8, AAV9 | AAV9, AAV10 | AAV10, AAV11 |
| AAV6, AAV8 | AAV7, AAV9 | AAV8, AAV10 | AAV9, AAV11 | AAV10, AAV12 |
| AAV6, AAV9 | AAV7, AAV10 | AAV8, AAV11 | AAV9, AAV12 | AAV10, AAVRH8 |
| AAV6, AAV10 | AAV7, AAV11 | AAV8, AAV12 | AAV9, AAVRH8 | AAV10, AAVRH10 |
| AAV6, AAV11 | AAV7, AAV12 | AAV8, AAVRH8 | AAV9, AAVRH10 | AAV10, AAV13 |
| AAV6, AAV12 | AAV7, AAVRH8 | AAV8, AAVRH10 | AAV9, AAV13 | AAV10, AAVDJ |
| AAV6, AAVRH8 | AAV7, AAVRH10 | AAV8, AAV13 | AAV9, AAVDJ | AAV10, AAVDJ8 |
| AAV6, AAVRH10 | AAV7, AAV13 | AAV8, AAVDJ | AAV9, AAVDJ8 | AAV10, AVIAN |
| AAV6, AAV13 | AAV7, AAVDJ | AAV8, AAVDJ8 | AAV9, AVIAN | AAV10, BOVINE |
| AAV6, AAVDJ | AAV7, AAVDJ8 | AAV8, AVIAN | AAV9, BOVINE | AAV10, CANINE |
| AAV6, AAVDJ8 | AAV7, AVIAN | AAV8, BOVINE | AAV9, CANINE | AAV10, EQUINE |
| AAV6, AVIAN | AAV7, BOVINE | AAV8, CANINE | AAV9, EQUINE | AAV10, GOAT |
| AAV6, BOVINE | AAV7, CANINE | AAV8, EQUINE | AAV9, GOAT | AAV10, SHRIMP |
| AAV6, CANINE | AAV7, EQUINE | AAV8, GOAT | AAV9, SHRIMP | AAV10, PORCINE |
| AAV6, EQUINE | AAV7, GOAT | AAV8, SHRIMP | AAV9, PORCINE | AAV10, INSECT |
| AAV6, GOAT | AAV7, SHRIMP | AAV8, PORCINE | AAV9, INSECT | AAV10, OVINE |
| AAV6, SHRIMP | AAV7, PORCINE | AAV8, INSECT | AAV9, OVINE | AAV10, B19 |
| AAV6, PORCINE | AAV7, INSECT | AAV8, OVINE | AAV9, B19 | AAV10, MVM |
| AAV6, INSECT | AAV7, OVINE | AAV8, B19 | AAV9, MVM | AAV10, GOOSE |
| AAV6, OVINE | AAV7, B19 | AAV8, MVM | AAV9, GOOSE | AAV10, SNAKE |
| AAV6, B19 | AAV7, MVM | AAV8, GOOSE | AAV9, SNAKE | |
| AAV6, MVM | AAV7, GOOSE | AAV8, SNAKE | | |
| AAV6, GOOSE | AAV7, SNAKE | | | |
| AAV6, SNAKE | | | | |
| AAV11, AAV11 | AAV12, AAV12 | AAVRH8, AAVRH8 | AAVRH10, AAVRH10 | AAV13, AAV13 |
| AAV11, AAV12 | AAV12, AAVRH8 | AAVRH8, AAVRH10 | AAVRH10, AAV13 | AAV13, AAVDJ |
| AAV11, AAVRH8 | AAV12, AAVRH10 | AAVRH8, AAV13 | AAVRH10, AAVDJ | AAV13, AAVDJ8 |
| AAV11, AAVRH10 | AAV12, AAV13 | AAVRH8, AAVDJ | AAVRH10, AAVDJ8 | AAV13, AVIAN |
| AAV11, AAV13 | AAV12, AAVDJ | AAVRH8, AAVDJ8 | AAVRH10, AVIAN | AAV13, BOVINE |
| AAV11, AAVDJ | AAV12, AAVDJ8 | AAVRH8, AVIAN | AAVRH10, BOVINE | AAV13, CANINE |
| AAV11, AAVDJ8 | AAV12, AVIAN | AAVRH8, BOVINE | AAVRH10, CANINE | AAV13, EQUINE |
| AAV11, AVIAN | AAV12, BOVINE | AAVRH8, CANINE | AAVRH10, EQUINE | AAV13, GOAT |
| AAV11, BOVINE | AAV12, CANINE | AAVRH8, EQUINE | AAVRH10, GOAT | AAV13, SHRIMP |
| AAV11, CANINE | AAV12, EQUINE | AAVRH8, GOAT | AAVRH10, SHRIMP | AAV13, PORCINE |
| AAV11, EQUINE | AAV12, GOAT | AAVRH8, SHRIMP | AAVRH10, PORCINE | AAV13, INSECT |
| AAV11, GOAT | AAV12, SHRIMP | AAVRH8, PORCINE | AAVRH10, INSECT | AAV13, OVINE |
| AAV11, SHRIMP | AAV12, PORCINE | AAVRH8, INSECT | AAVRH10, OVINE | AAV13, B19 |
| AAV11, PORCINE | AAV12, INSECT | AAVRH8, OVINE | AAVRH10, B19 | AAV13, MVM |
| AAV11, INSECT | AAV12, OVINE | AAVRH8, B19 | AAVRH10, MVM | AAV13, GOOSE |
| AAV11, OVINE | AAV12, B19 | AAVRH8, MVM | AAVRH10, GOOSE | AAV13, SNAKE |
| AAV11, B19 | AAV12, MVM | AAVRH8, GOOSE | AAVRH10, SNAKE | |
| AAV11, MVM | AAV12, GOOSE | AAVRH8, SNAKE | | |
| AAV11, GOOSE | AAV12, SNAKE | | | |
| AAV11, SNAKE | | | | |
| AAVDJ, AAVDJ | AAVDJ8, AVVDJ8 | AVIAN, AVIAN | BOVINE, BOVINE | CANINE, CANINE |
| AAVDJ, AAVDJ8 | AAVDJ8, AVIAN | AVIAN, BOVINE | BOVINE, CANINE | CANINE, EQUINE |
| AAVDJ, AVIAN | AAVDJ8, BOVINE | AVIAN, CANINE | BOVINE, EQUINE | CANINE, GOAT |
| AAVDJ, BOVINE | AAVDJ8, CANINE | AVIAN, EQUINE | BOVINE, GOAT | CANINE, SHRIMP |
| AAVDJ, CANINE | AAVDJ8, EQUINE | AVIAN, GOAT | BOVINE, SHRIMP | CANINE, PORCINE |
| AAVDJ, EQUINE | AAVDJ8, GOAT | AVIAN, SHRIMP | BOVINE, PORCINE | CANINE, INSECT |
| AAVDJ, GOAT | AAVDJ8, SHRIMP | AVIAN, PORCINE | BOVINE, INSECT | CANINE, OVINE |
| AAVDJ, SHRIMP | AAVDJ8, PORCINE | AVIAN, INSECT | BOVINE, OVINE | CANINE, B19 |
| AAVDJ, PORCINE | AAVDJ8, INSECT | AVIAN, OVINE | BOVINE, B19 | CANINE, MVM |
| AAVDJ, INSECT | AAVDJ8, OVINE | AVIAN, B19 | BOVINE, MVM | CANINE, GOOSE |
| AAVDJ, OVINE | AAVDJ8, B19 | AVIAN, MVM | BOVINE, GOOSE | CANINE, SNAKE |
| AAVDJ, B19 | AAVDJ8, MVM | AVIAN, GOOSE | BOVINE, SNAKE | |
| AAVDJ, MVM | AAVDJ8, GOOSE | AVIAN, SNAKE | | |
| AAVDJ, GOOSE | AAVDJ8, SNAKE | | | |
| AAVDJ, SNAKE | | | | |
| EQUINE, EQUINE | GOAT, GOAT | SHRIMP, SHRIMP | PORCINE, PORCINE | INSECT, INSECT |
| EQUINE, GOAT | GOAT, SHRIMP | SHRIMP, PORCINE | PORCINE, INSECT | INSECT, OVINE |
| EQUINE, SHRIMP | GOAT, PORCINE | SHRIMP, INSECT | PORCINE, OVINE | INSECT, B19 |
| EQUINE, PORCINE | GOAT, INSECT | SHRIMP, OVINE | PORCINE, B19 | INSECT, MVM |
| EQUINE, INSECT | GOAT, OVINE | SHRIMP, B19 | PORCINE, MVM | INSECT, GOOSE |
| EQUINE, OVINE | GOAT, B19 | SHRIMP, MVM | PORCINE, GOOSE | INSECT, SNAKE |
| EQUINE, B19 | GOAT, MVM | SHRIMP, GOOSE | PORCINE, SNAKE | |
| EQUINE, MVM | GOAT, GOOSE | SHRIMP, SNAKE | | |
| EQUINE, GOOSE | GOAT, SNAKE | | | |
| EQUINE, SNAKE | | | | |
| OVINE, OVINE | B19, B19 | MVM, MVM | GOOSE, GOOSE | SNAKE, SNAKE |
| OVINE, B19 | B19, MVM | MVM, GOOSE | GOOSE, SNAKE | |
| OVINE, MVM | B19, GOOSE | MVM, SNAKE | | |
| OVINE, GOOSE | B19, SNAKE | | | |
| OVINE, SNAKE | | | | |

By way of example only, Table 2 shows the sequences of exemplary WT-ITRs from some different AAV serotypes.

TABLE 2

| AAV | 5′ WT-ITR | 3′ WT-ITR |
|---|---|---|
| serotype | (LEFT) | (RIGHT) |
| AAV1 | 5′-TTGCCCACTCCCTCT | 5′-TTACCCTAGTGATGG |
| | CTGCGCGCTCGCTCG | AGTTGCCCACTCCCT |
| | CTCGGTGGGGCCTGC | CTCTGCGCGCGTCGC |
| | GGACCAAAGGTCCGC | TCGCTCGGTGGGGCC |
| | AGACGGCAGAGGTCT | GGCAGAGGAGACCTC |
| | CCTCTGCCGGCCCCA | TGCCGTCTGCGGACC |
| | CCGAGCGAGCGACGC | TTTGGTCCGCAGGCC |
| | GCGCAGAGAGGGAGT | CCACCGAGCGAGCGA |
| | GGGCAACTCCATCAC | GCGCGCAGAGAGGGA |
| | TAGGGTAA-3′ | GTGGGCAA-3′ |
| | (SEQ ID NO: 560) | (SEQ ID NO: 565) |
| | | (from Kay et al., |
| | | J Virol, 2006, |
| | | 426-439, FIG.1A) |
| AAV2 | CCTGCAGGCAGCTGC | AGGAACCCCTAGTGA |
| | GCGCTCGCTCGCTCA | TGGAGTTGGCCACTC |
| | CTGAGGCCGCCCGGG | CCTCTCTGCGCGCTC |
| | CAAAGCCCGGGCGTC | GCTCGCTCACTGAGG |
| | GGGCGACCTTTGGTC | CCGGGCGACCAAAGG |
| | GCCCGGCCTCAGTGA | TCGCCCGACGCCCGG |
| | GCGAGCGAGCGCGCA | GCTTTGCCCGGGCGG |
| | GAGAGGGAGTGGCCA | CCTCAGTGAGCGAGC |
| | ACTCCATCACTAGGG | GAGCGCGCAGCTGCC |
| | GTTCCT | TGCAGG |
| | (SEQ ID NO: 51) | (SEQ ID NO: 1) |
| AAV3 | 5′-TTGGCCACTCCCTCT | 5′-ATACCTCTAGTGATG |
| | ATGCGCACTCGCTCG | GAGTTGGCCACTCCC |
| | CTCGGTGGGGCCTGG | TCTATGCGCACTCGC |
| | CGACCAAAGGTCGCC | TCGCTCGGTGGGGCC |
| | AGACGGACGTGGGTT | GGACGTGGAAACCCA |
| | TCCACGTCCGGCCCC | CGTCCGTCTGGCGAC |
| | ACCGAGCGAGCGAGT | CTTTGGTCGCCAGGC |
| | GCGCATAGAGGGAGT | CCCACCGAGCGAGCG |
| | GGCCAACTCCATCAC | AGTGCGCATAGAGGG |
| | TAGAGGTAT-3′ | AGTGGCCAA-3′ |
| | (SEQ ID NO: 561) | (SEQ ID NO: 566) |
| | | (from Kay et al., |
| | | J Virol, 2006, |
| | | 426-439,FIG. 1A) |
| AAV4 | 5′-TTGGCCACTCCCTCT | 5′-AGTTGGCCACATTAG |
| | ATGCGCGCTCGCTCA | CTATGCGCGCTCGCT |
| | CTCACTCGGCCCTGG | CACTCACTCGGCCCT |
| | AGACCAAAGGTCTCC | GGAGACCAAAGGTCT |
| | AGACTGCCGGCCTCT | CCAGACTGCCGGCCT |
| | GGCCGGCAGGGCCGA | CTGGCCGGCAGGGCC |
| | GTGAGTGAGCGAGCG | GAGTGAGTGAGCGAG |
| | CGCATAGAGGGAGTG | CGCGCATAGAGGGAG |
| | GCCAACT-3′ | TGGCCAA-3′ |
| | (SEQ ID NO: 562) | (SEQ ID NO: 567) |
| AAV5 | 5′-TCCCCCCTGTCGCGT | 5′-CTTACAAAACCCCCT |
| | TCGCTCGCTCGCTGG | TGCTTGAGAGTGTGG |
| | CTCGTTTGGGGGGGC | CACTCTCCCCCCTGT |
| | GACGGCCAGAGGGCC | CGCGTTCGCTCGCTC |
| | GTCGTCTGGCAGCTC | GCTGGCTCGTTTGGG |
| | TTTGAGCTGCCACCC | GGGGTGGCAGCTCAA |
| | CCCCAAACGAGCCAG | AGAGCTGCCAGACGA |
| | CGAGCGAGCGAACGC | CGGCCCTCTGGCCGT |
| | GACAGGGGGGAGAGT | CGCCCCCCCAAACGA |
| | GCCACACTCTCAAGC | GCCAGCGAGCGAGCG |
| | AAGGGGGTTTTGTAA | AACGCGACAGGGGGG |
| | G-3′ | A-3′ |
| | (SEQ ID NO: 563) | (SEQ ID NO: 568) |
| AAV6 | 5′-TTGCCCACTCCCTCT | 5′-ATACCCCTAGTGATG |
| | AATGCGCGCTCGCTC | GAGTTGCCCACTCCC |
| | GCTCGGTGGGGCCTG | TCTATGCGCGCTCGC |
| | CGGACCAAAGGTCCG | TCGCTCGGTGGGGCC |
| | CAGACGGCAGAGGTC | GGCAGAGGAGACCTC |
| | TCCTCTGCCGGCCCC | TGCCGTCTGCGGACC |
| | ACCGAGCGAGCGAGC | TTTGGTCCGCAGGCC |
| | GCGCATAGAGGGAGT | CCACCGAGCGAGCGA |
| | GGGCAACTCCATCAC | GCGCGCATTAGAGGG |
| | TAGGGGTAT-3′ | AGTGGGCAA |
| | (SEQ ID NO: 564) | (SEQ ID NO: 569) |
| | | (from Kay et al., |
| | | J Virol, 2006, |
| | | 426-439,FIG. 1A) |

In some embodiments, the nucleotide sequence of the WT-ITR sequence can be modified (e.g., by modifying 1, 2, 3, 4 or 5, or more nucleotides or any range therein), whereby the modification is a substitution for a complementary nucleotide, e.g., G for a C, and vice versa, and T for an A, and vice versa.

In certain embodiments of the present invention, the ceDNA vector does not have a WT-ITR consisting of the nucleotide sequence selected from any of: SEQ ID NOs: 550-557.

In alternative embodiments of the present invention, if a ceDNA vector has a WT-ITR comprising the nucleotide sequence selected from any of: SEQ ID NOs: 550-557, then the flanking ITR is also a WT and the cDNA comprises a regulatory switch, e.g., as disclosed herein and in PCT/US18/49996 (e.g., see Table 11 of PCT/US18/49996). In some embodiments, the ceDNA vector comprises a regulatory switch as disclosed herein and a WT-ITR selected having the nucleotide sequence selected from any of the group consisting of: SEQ ID NO: 550-557.

The ceDNA vector described herein can include WT-ITR structures that retains an operable RBE, trs and RBE′ portion. FIG. 2A and FIG. 2B, using wild-type ITRs for exemplary purposes, show one possible mechanism for the operation of a trs site within a wild type ITR structure portion of a ceDNA vector. In some embodiments, the ceDNA vector contains one or more functional WT-ITR polynucleotide sequences that comprise a Rep-binding site (RBS; 5′-GCGCGCTCGCTCGCTC-3′ (SEQ ID NO: 531) for AAV2) and a terminal resolution site (TRS; 5′-AGTT (SEQ ID NO: 46)). In some embodiments, at least one WT-ITR is functional. In alternative embodiments, where a ceDNA vector comprises two WT-ITRs that are substantially symmetrical to each other, at least one WT-ITR is functional and at least one WT-ITR is non-functional.

B. Modified ITRs (Mod-ITRs) in General for ceDNA Vectors Comprising Asymmetric ITR Pairs or Symmetric ITR Pairs

As discussed herein, a ceDNA vector can comprise a symmetrical ITR pair or an asymmetrical ITR pair. In both instances, the ITRs can be modified ITRs—the difference being that in the first instance (i.e., symmetric mod-ITRs), the mod-ITRs have the same three-dimensional spatial organization (i.e., have the same A-A′, C-C′ and B-B′ arm configurations), whereas in the second instance (i.e., asymmetric mod-ITRs), the mod-ITRs have a different three-dimensional spatial organization (i.e., have a different configuration of A-A′, C-C′ and B-B′ arms).

In some embodiments, a modified ITR is an ITRs that is modified by deletion, insertion, and/or substitution as compared to a wild-type ITR sequence (e.g. AAV ITR). In some embodiments, at least one of the ITRs in the ceDNA vector comprises a functional Rep binding site (RBS; e.g. 5′-GCGCGCTCGCTCGCTC-3′ for AAV2, SEQ ID NO: 531) and a functional terminal resolution site (TRS; e.g. 5′-AGTT-3′, SEQ ID NO: 46.) In one embodiment, at least one of the ITRs is a non-functional ITR. In one embodiment, the different or modified ITRs are not each wild type ITRs from different serotypes.

Specific alterations and mutations in the ITRs are described in detail herein, but in the context of ITRs, “altered” or “mutated” or “modified”, it indicates that nucleotides have been inserted, deleted, and/or substituted relative to the wild-type, reference, or original ITR sequence. The altered or mutated ITR can be an engineered ITR. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature.

In some embodiments, a mod-ITR may be synthetic. In one embodiment, a synthetic ITR is based on ITR sequences from more than one AAV serotype. In another embodiment, a synthetic ITR includes no AAV-based sequence. In yet another embodiment, a synthetic ITR preserves the ITR structure described above although having only some or no AAV-sourced sequence. In some aspects, a synthetic ITR may interact preferentially with a wild type Rep or a Rep of a specific serotype, or in some instances will not be recognized by a wild-type Rep and be recognized only by a mutated Rep.

The skilled artisan can determine the corresponding sequence in other serotypes by known means. For example, determining if the change is in the A, A′, B, B′, C, C′ or D region and determine the corresponding region in another serotype. One can use BLAST® (Basic Local Alignment Search Tool) or other homology alignment programs at default status to determine the corresponding sequence. The invention further provides populations and pluralities of ceDNA vectors comprising mod-ITRs from a combination of different AAV serotypes—that is, one mod-ITR can be from one AAV serotype and the other mod-ITR can be from a different serotype. Without wishing to be bound by theory, in one embodiment one ITR can be from or based on an AAV2 ITR sequence and the other ITR of the ceDNA vector can be from or be based on any one or more ITR sequence of AAV serotype 1 (AAV1), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), AAV serotype 11 (AAV11), or AAV serotype 12 (AAV12).

Any parvovirus ITR can be used as an ITR or as a base ITR for modification. Preferably, the parvovirus is a dependovirus. More preferably AAV. The serotype chosen can be based upon the tissue tropism of the serotype. AAV2 has a broad tissue tropism, AAV1 preferentially targets to neuronal and skeletal muscle, and AAV5 preferentially targets neuronal, retinal pigmented epithelia, and photoreceptors. AAV6 preferentially targets skeletal muscle and lung. AAV8 preferentially targets liver, skeletal muscle, heart, and pancreatic tissues. AAV9 preferentially targets liver, skeletal and lung tissue. In one embodiment, the modified ITR is based on an AAV2 ITR.

More specifically, the ability of a structural element to functionally interact with a particular large Rep protein can be altered by modifying the structural element. For example, the nucleotide sequence of the structural element can be modified as compared to the wild-type sequence of the ITR. In one embodiment, the structural element (e.g., A arm, A′ arm, B arm, B′ arm, C arm, C′ arm, D arm, RBE, RBE′, and trs) of an ITR can be removed and replaced with a wild-type structural element from a different parvovirus. For example, the replacement structure can be from AAV1, AAV2, AAV3, AAV4, AAV5, AAV6, AAV7, AAV8, AAV9, AAV10, AAV11, AAV12, AAV13, snake parvovirus (e.g., royal python parvovirus), bovine parvovirus, goat parvovirus, avian parvovirus, canine parvovirus, equine parvovirus, shrimp parvovirus, porcine parvovirus, or insect AAV. For example, the ITR can be an AAV2 ITR and the A or A′ arm or RBE can be replaced with a structural element from AAV5. In another example, the ITR can be an AAV5 ITR and the C or C′ arms, the RBE, and the trs can be replaced with a structural element from AAV2. In another example, the AAV ITR can be an AAV5 ITR with the B and B′ arms replaced with the AAV2 ITR B and B′ arms.

By way of example only, Table 3 indicates exemplary modifications of at least one nucleotide (e.g., a deletion, insertion and/or substitution) in regions of a modified ITR, where X is indicative of a modification of at least one nucleic acid (e.g., a deletion, insertion and/or substitution) in that section relative to the corresponding wild-type ITR. In some embodiments, any modification of at least one nucleotide (e.g., a deletion, insertion and/or substitution) in any of the regions of C and/or C′ and/or B and/or B′ retains three sequential T nucleotides (i.e., TTT) in at least one terminal loop. For example, if the modification results in any of: a single arm ITR (e.g., single C-C′ arm, or a single B-B′ arm), or a modified C-B′ arm or C′-B arm, or a two arm ITR with at least one truncated arm (e.g., a truncated C-C′ arm and/or truncated B-B′ arm), at least the single arm, or at least one of the arms of a two arm ITR (where one arm can be truncated) retains three sequential T nucleotides (i.e., TTT) in at least one terminal loop. In some embodiments, a truncated C-C′ arm and/or a truncated B-B′ arm has three sequential T nucleotides (i.e., TTT) in the terminal loop.

TABLE 3
Exemplary combinations of modifications of at least one nucleotide (e.g., a
deletion, insertion and/or substitution) to different B-B′ and C-C′ regions or
arms of ITRs (X indicates a nucleotide modification, e.g., addition, deletion
or substitution of at least one nucleotide in the region).

| B region | B′ region | C region | C′ region |
|---|---|---|---|
| X | | | |
| | X | | |
| X | X | | |
| | | X | |
| | | | X |
| | | X | X |
| X | | X | |
| X | | | X |
| | X | X | |
| | X | | X |
| X | X | X | |
| X | X | | X |
| X | | X | X |
| | X | X | X |
| X | X | X | X |

In some embodiments, mod-ITR for use in a gene editing ceDNA vector comprising an asymmetric ITR pair, or a symmetric mod-ITR pair as disclosed herein can comprise any one of the combinations of modifications shown in Table 3, and also a modification of at least one nucleotide in any one or more of the regions selected from: between A′ and C, between C and C′, between C′ and B, between B and B′ and between B′ and A. In some embodiments, any modification of at least one nucleotide (e.g., a deletion, insertion and/or substitution) in the C or C′ or B or B′ regions, still preserves the terminal loop of the stem-loop. In some embodiments, any modification of at least one nucleotide (e.g., a deletion, insertion and/or substitution) between C and C′ and/or B and B′ retains three sequential T nucleotides (i.e., TTT) in at least one terminal loop. In alternative embodiments, any modification of at least one nucleotide (e.g., a deletion, insertion and/or substitution) between C and C′ and/or B and B′ retains three sequential A nucleotides (i.e., AAA) in at least one terminal loop In some embodiments, a modified ITR for use herein can comprise any one of the combinations of modifications shown in Table 3, and also a modification of at least one nucleotide (e.g., a deletion, insertion and/or substitution) in any one or more of the regions selected from: A′, A and/or D. For example, in some embodiments, a modified ITR for use herein can comprise any one of the combinations of modifications shown in Table 3, and also a modification of at least one nucleotide (e.g., a deletion, insertion and/or substitution) in the A region. In some embodiments, a modified ITR for use herein can comprise any one of the combinations of modifications shown in Table 3, and also a modification of at least one nucleotide (e.g., a deletion, insertion and/or substitution) in the A′ region. In some embodiments, a modified ITR for use herein can comprise any one of the combinations of modifications shown in Table 3, and also a modification of at least one nucleotide (e.g., a deletion, insertion and/or substitution) in the A and/or A′ region. In some embodiments, a modified ITR for use herein can comprise any one of the combinations of modifications shown in Table 3, and also a modification of at least one nucleotide (e.g., a deletion, insertion and/or substitution) in the D region.

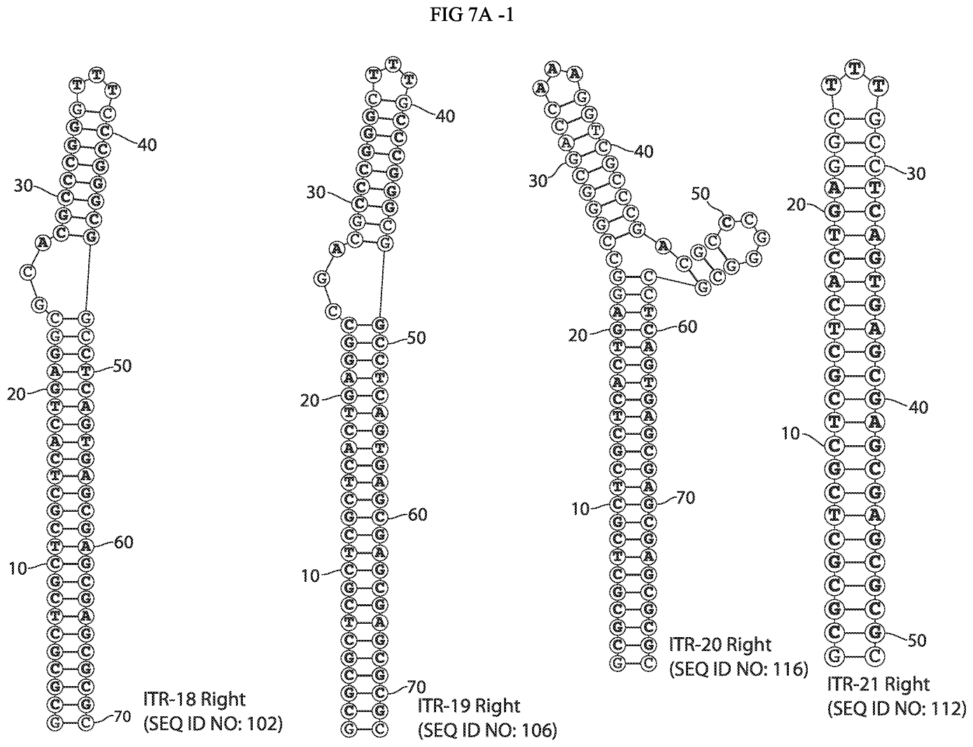

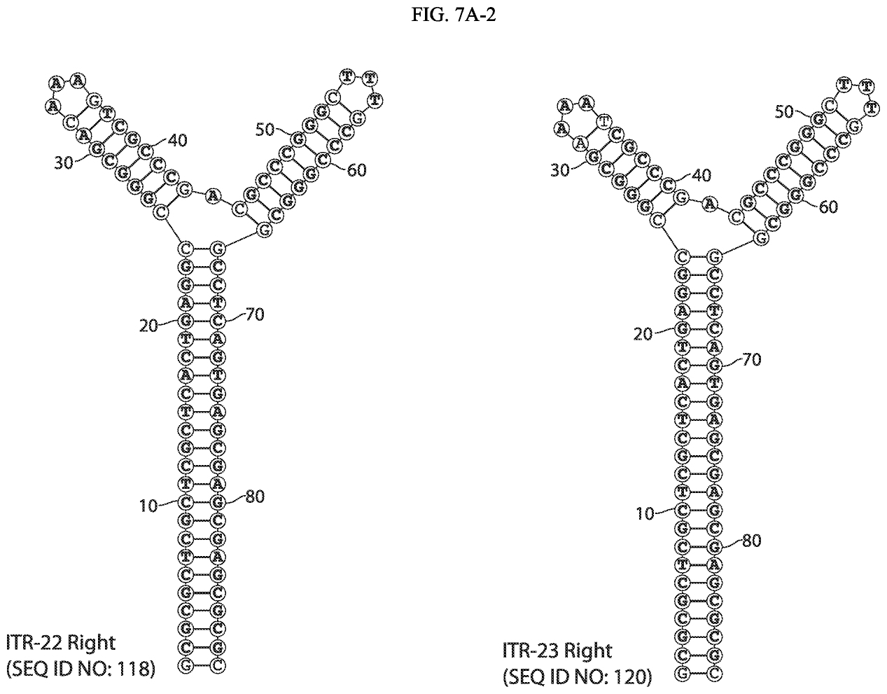

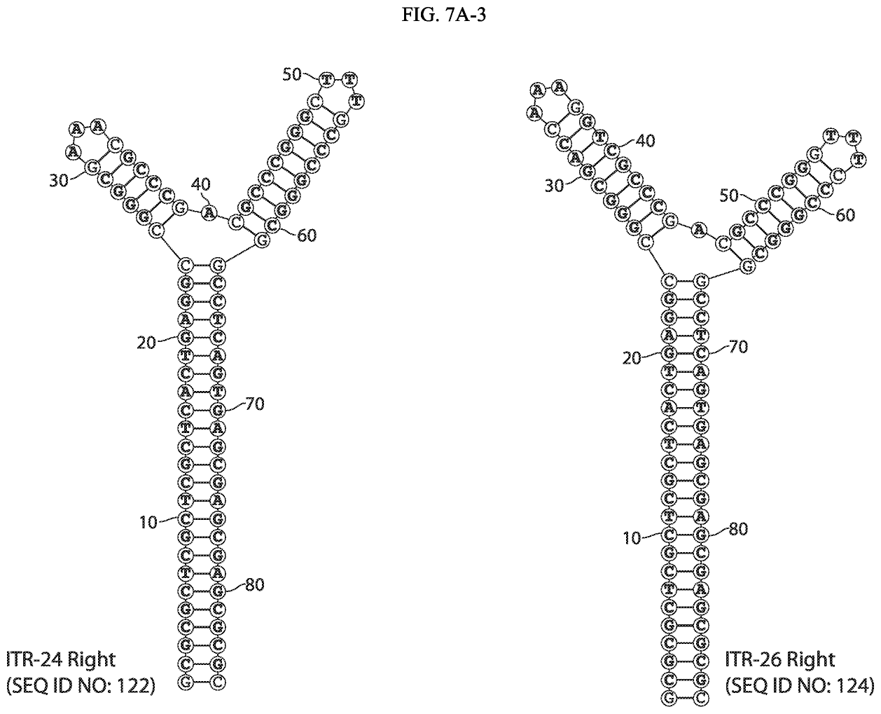

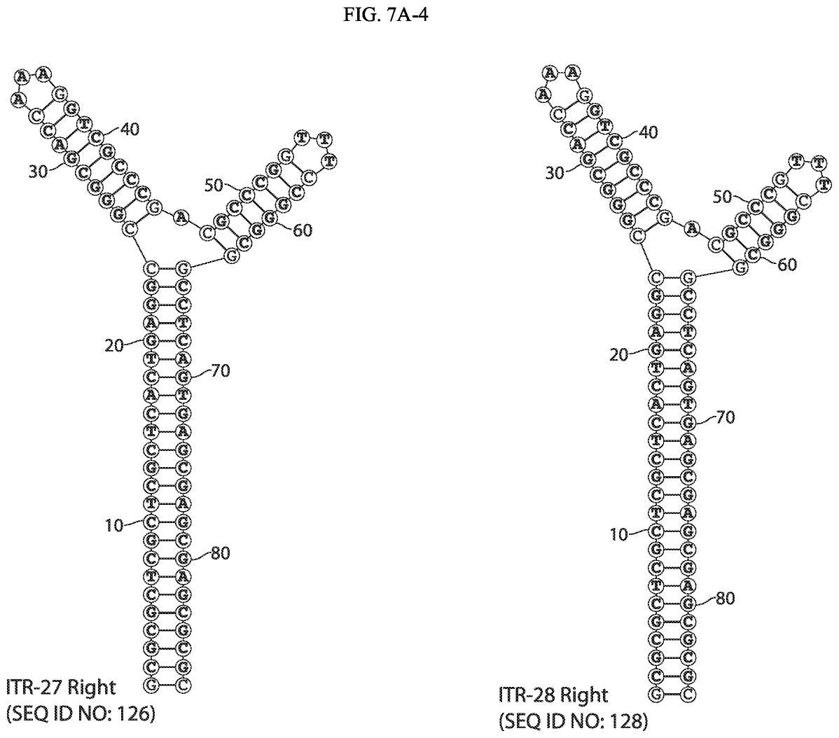

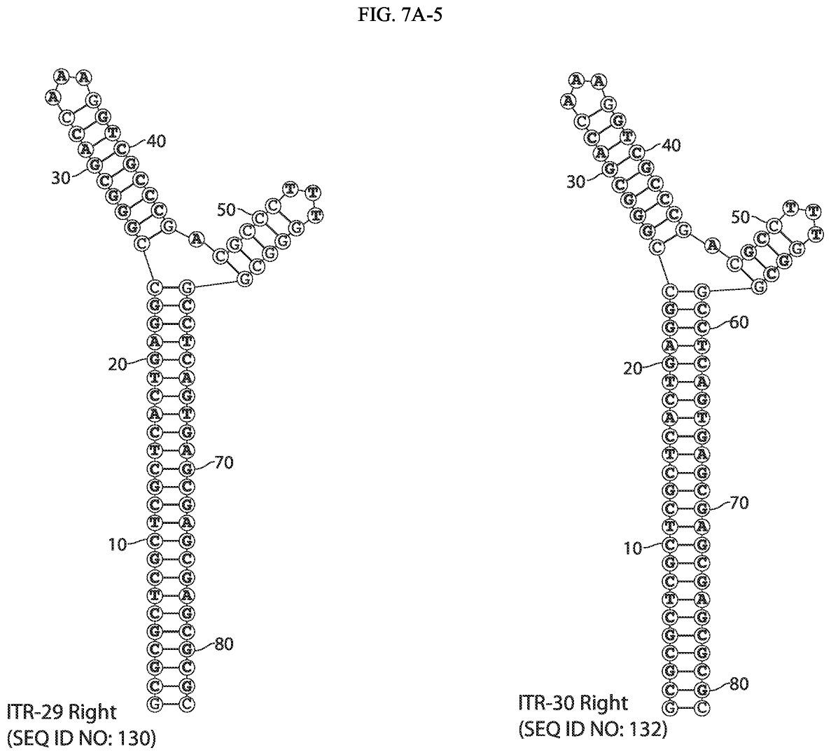

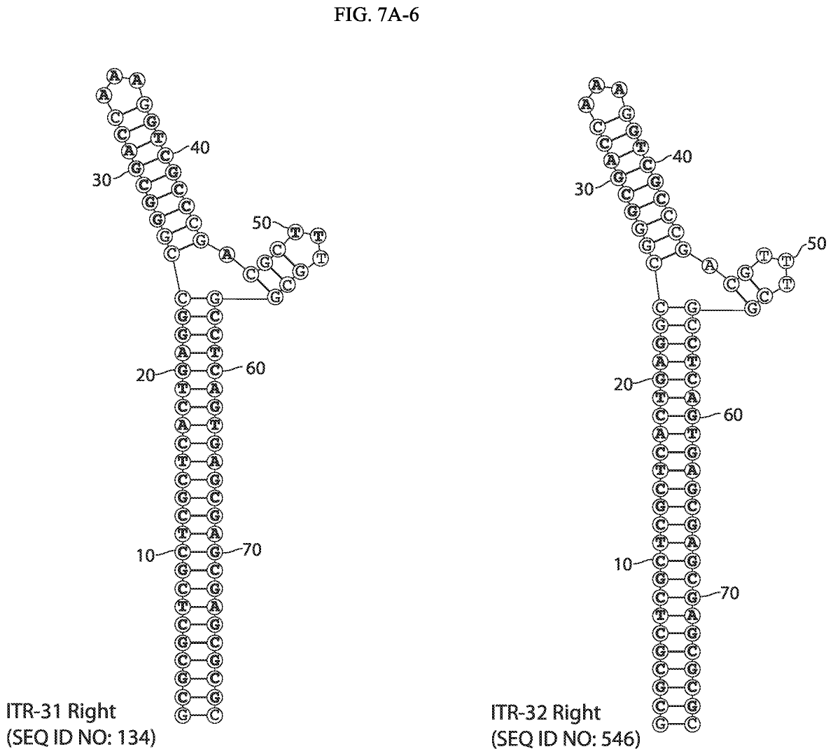

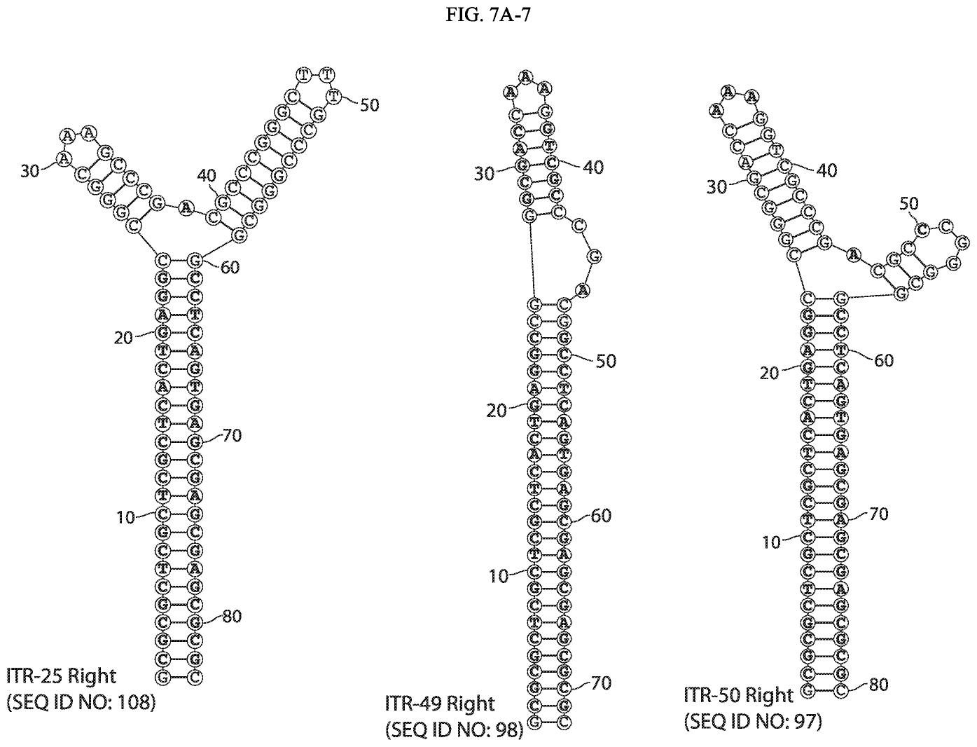

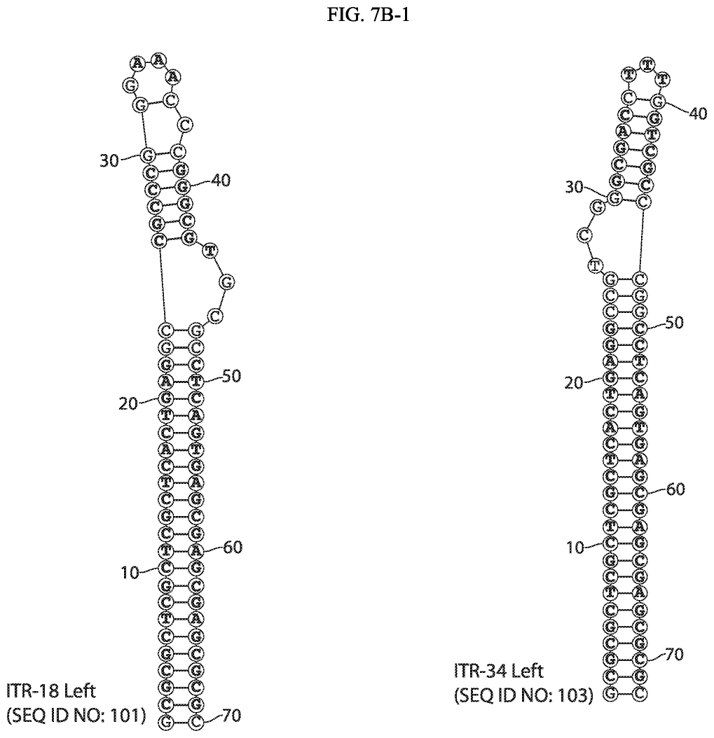

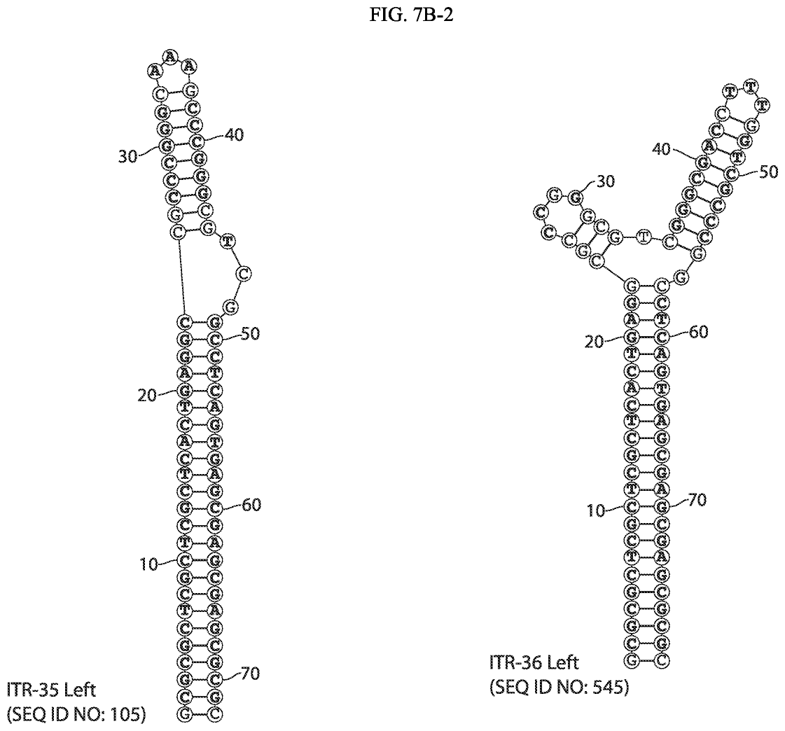

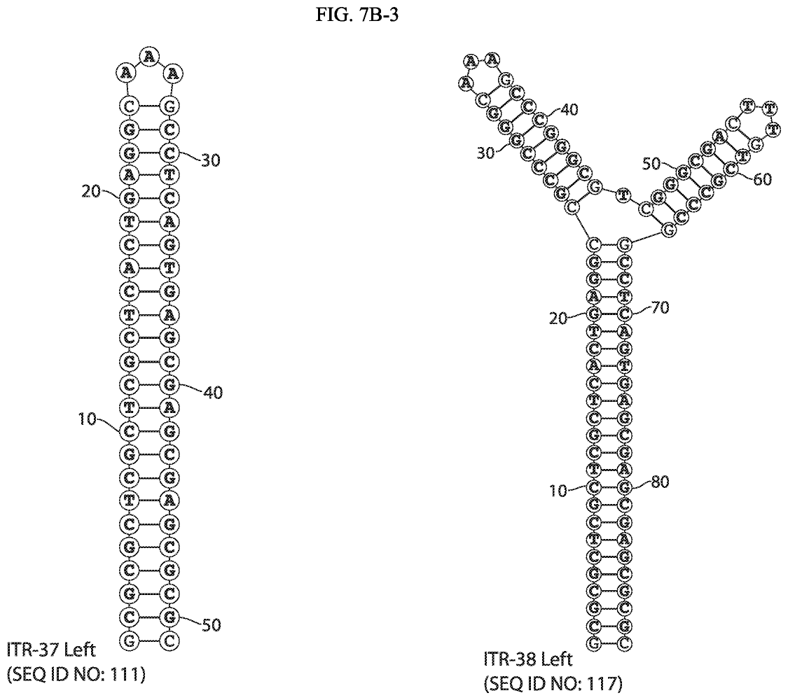

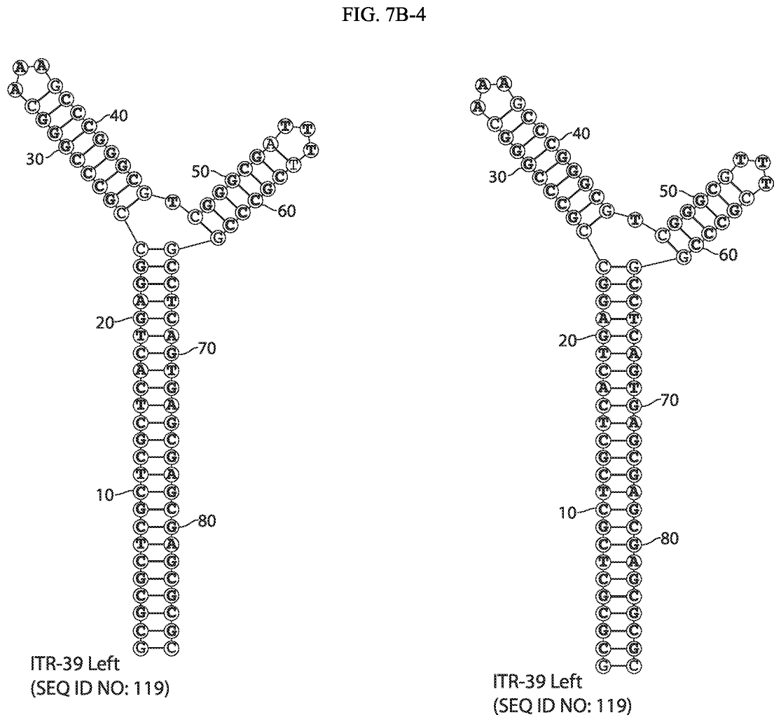

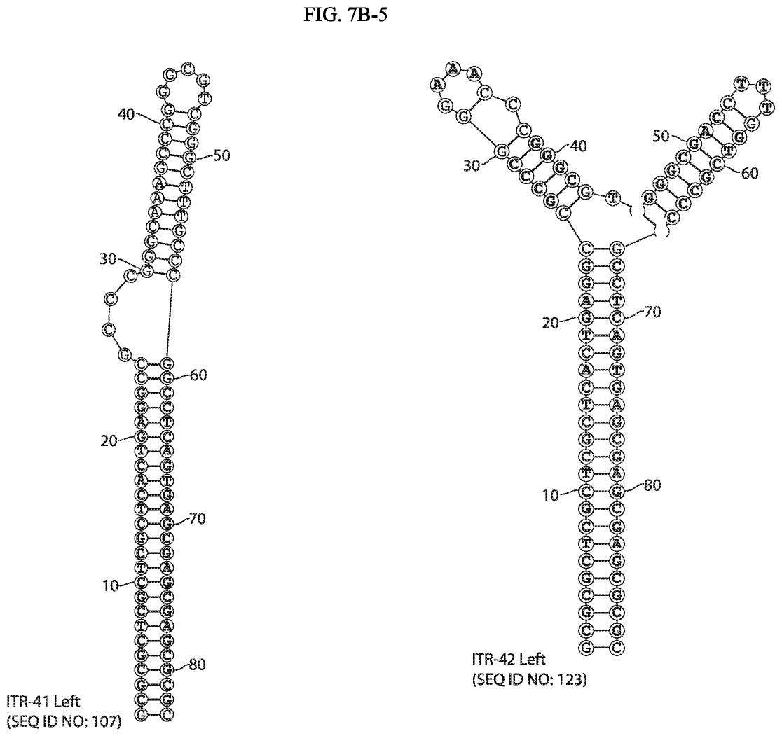

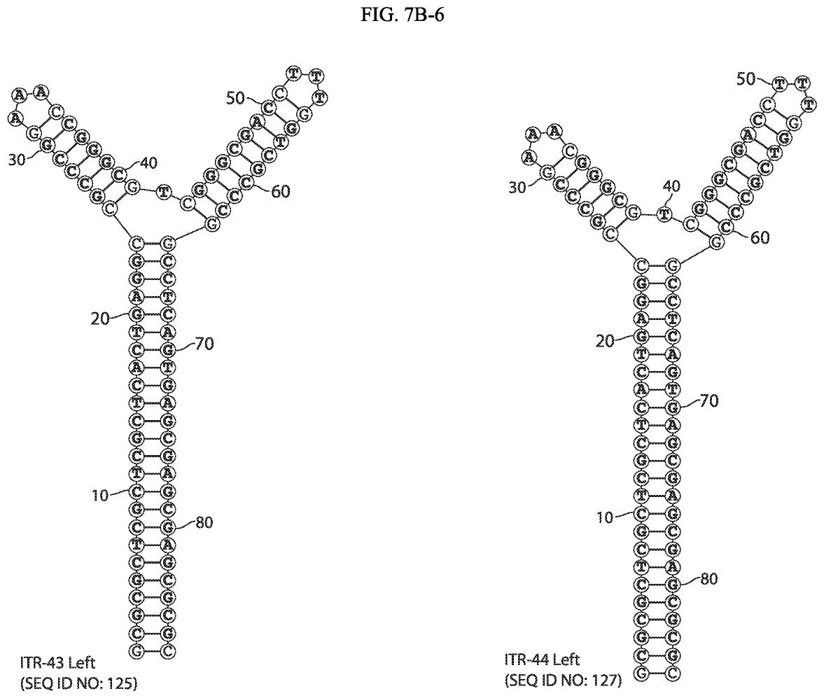

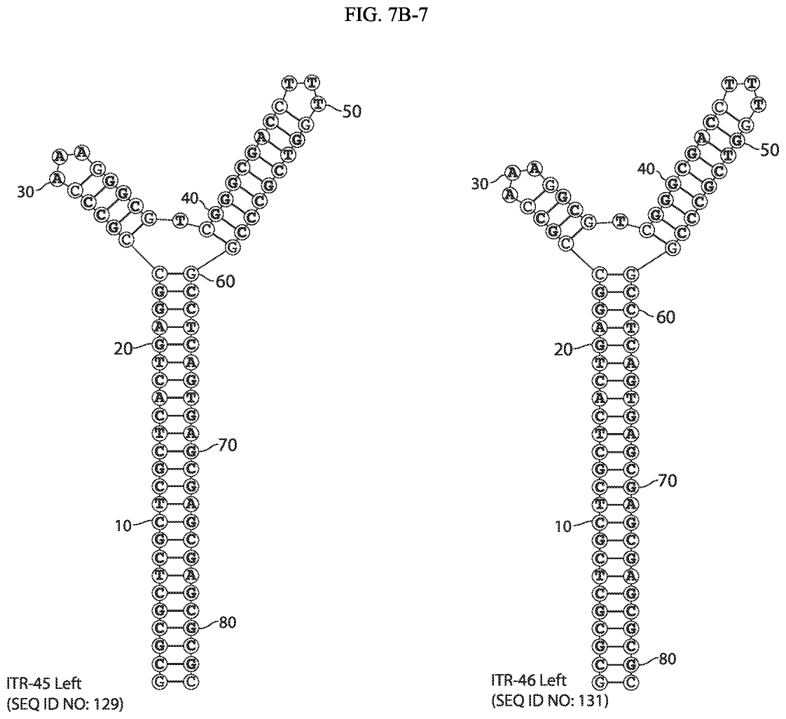

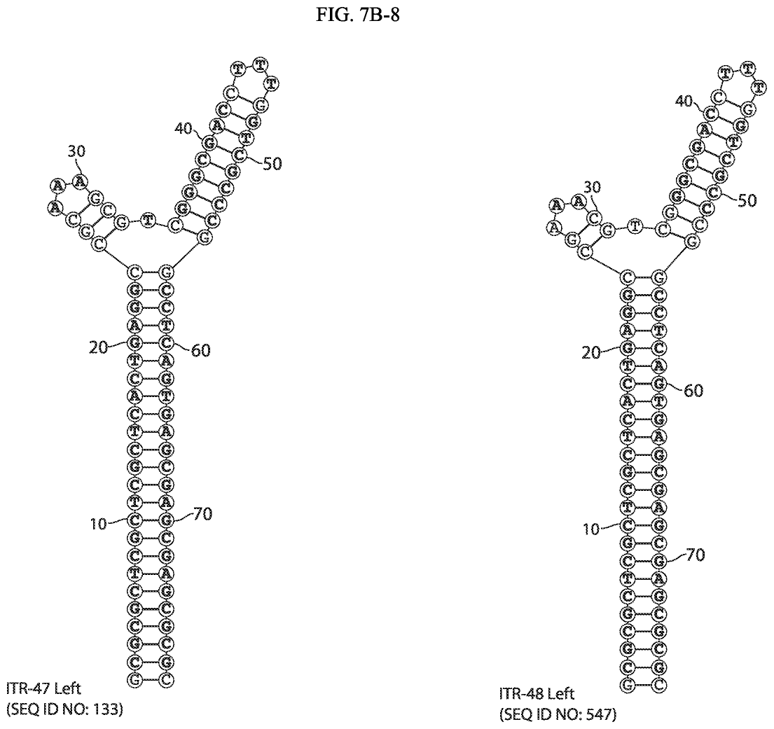

In one embodiment, the nucleotide sequence of the structural element can be modified (e.g., by modifying 1, 2, 3, 4, 5, 6, 7, 8, 9, 10, 11, 12, 13, 14, 15, 16, 17, 18, 19, or 20 or more nucleotides or any range therein) to produce a modified structural element. In one embodiment, the specific modifications to the ITRs are exemplified herein (e.g., SEQ ID NOS: 2, 52, 63, 64, 99-100, 469-499, or shown in in FIG. 7A-7B herein (e.g., 97-98, 101-103, 105-108, 111-112, 117-134, 545-54). In some embodiments, an ITR can be modified (e.g., by modifying 1, 2, 3, 4, 5, 6, 7, 8, 9, 10, 11, 12, 13, 14, 15, 16, 17, 18, 19, or 20 or more nucleotides or any range therein). In other embodiments, the ITR can have at least 80%, at least 85%, at least 90%, at least 95%, at least 96%, at least 97%, at least 98%, at least 99%, or more sequence identity with one of the modified ITRs of SEQ ID NOS: 469-499 or 545-547, or the RBE-containing section of the A-A′ arm and C-C′ and B-B′ arms of SEQ ID NO: 97-98, 101-103, 105-108, 111-112, 117-134, 545-547, or shown in Tables 2-9 (i.e., SEQ ID NO: 110-112, 115-190, 200-468) of PCT/US18/49996, which is incorporated herein in its entirety by reference.

In some embodiments, a modified ITR can for example, comprise removal or deletion of all of a particular arm, e.g., all or part of the A-A′ arm, or all or part of the B-B′ arm or all or part of the C-C′ arm, or alternatively, the removal of 1, 2, 3, 4, 5, 6, 7, 8, 9 or more base pairs forming the stem of the loop so long as the final loop capping the stem (e.g., single arm) is still present (e.g., see ITR-21 in FIG. 7A). In some embodiments, a modified ITR can comprise the removal of 1, 2, 3, 4, 5, 6, 7, 8, 9 or more base pairs from the B-B′ arm. In some embodiments, a modified ITR can comprise the removal of 1, 2, 3, 4, 5, 6, 7, 8, 9 or more base pairs from the C-C′ arm (see, e.g., ITR-1 in FIG. 3B, or ITR-45 in FIG. 7A). In some embodiments, a modified ITR can comprise the removal of 1, 2, 3, 4, 5, 6, 7, 8, 9 or more base pairs from the C-C′ arm and the removal of 1, 2, 3, 4, 5, 6, 7, 8, 9 or more base pairs from the B-B′ arm. Any combination of removal of base pairs is envisioned, for example, 6 base pairs can be removed in the C-C′ arm and 2 base pairs in the B-B′ arm. As an illustrative example, FIG. 3B shows an exemplary modified ITR with at least 7 base pairs deleted from each of the C portion and the C′ portion, a substitution of a nucleotide in the loop between C and C′ region, and at least one base pair deletion from each of the B region and B′ regions such that the modified ITR comprises two arms where at least one arm (e.g., C-C′) is truncated. In some embodiments, the modified ITR also comprises at least one base pair deletion from each of the B region and B′ regions, such that the B-B′ arm is also truncated relative to WT ITR.

In some embodiments, a modified ITR can have between 1 and 50 (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) nucleotide deletions relative to a full-length wild-type ITR sequence. In some embodiments, a modified ITR can have between 1 and 30 nucleotide deletions relative to a full-length WT ITR sequence. In some embodiments, a modified ITR has between 2 and 20 nucleotide deletions relative to a full-length wild-type ITR sequence.

In some embodiments, a modified ITR does not contain any nucleotide deletions in the RBE-containing portion of the A or A′ regions, so as not to interfere with DNA replication (e.g. binding to a RBE by Rep protein, or nicking at a terminal resolution site). In some embodiments, a modified ITR encompassed for use herein has one or more deletions in the B, B′, C, and/or C region as described herein.

In some embodiments, the gene editing ceDNA vector comprising a symmetric ITR pair or asymmetric ITR pair comprises a regulatory switch as disclosed herein and at least one modified ITR selected having the nucleotide sequence selected from any of the group consisting of: SEQ ID NO: 550-557.

In another embodiment, the structure of the structural element can be modified. For example, the structural element a change in the height of the stem and/or the number of nucleotides in the loop. For example, the height of the stem can be about 2, 3, 4, 5, 6, 7, 8, or 9 nucleotides or more or any range therein. In one embodiment, the stem height can be about 5 nucleotides to about 9 nucleotides and functionally interacts with Rep. In another embodiment, the stem height can be about 7 nucleotides and functionally interacts with Rep. In another example, the loop can have 3, 4, 5, 6, 7, 8, 9, or 10 nucleotides or more or any range therein.

In another embodiment, the number of GAGY binding sites or GAGY-related binding sites within the RBE or extended RBE can be increased or decreased. In one example, the RBE or extended RBE, can comprise 1, 2, 3, 4, 5, or 6 or more GAGY binding sites or any range therein. Each GAGY binding site can independently be an exact GAGY sequence or a sequence similar to GAGY as long as the sequence is sufficient to bind a Rep protein.

In another embodiment, the spacing between two elements (such as but not limited to the RBE and a hairpin) can be altered (e.g., increased or decreased) to alter functional interaction with a large Rep protein. For example, the spacing can be about 1, 2, 3, 4, 5, 6, 7, 8, 9, 10, 11, 12, 13, 14, 15, 16, 17, 18, 19, 20, or 21 nucleotides or more or any range therein.

The ceDNA vector described herein can include an ITR structure that is modified with respect to the wild type AAV2 ITR structure disclosed herein, but still retains an operable RBE, trs and RBE′ portion. FIG. 2A and FIG. 2B show one possible mechanism for the operation of a trs site within a wild type ITR structure portion of a ceDNA vector. In some embodiments, the ceDNA vector contains one or more functional ITR polynucleotide sequences that comprise a Rep-binding site (RBS; 5′-GCGCGCTCGCTCGCTC-3′ (SEQ ID NO: 531) for AAV2) and a terminal resolution site (TRS; 5′-AGTT (SEQ ID NO: 46)). In some embodiments, at least one ITR (wt or modified ITR) is functional. In alternative embodiments, where a ceDNA vector comprises two modified ITRs that are different or asymmetrical to each other, at least one modified ITR is functional and at least one modified ITR is non-functional.

In some embodiments, a ceDNA vector does not have a modified ITR selected from any sequence consisting of, or consisting essentially of: SEQ ID NOs:500-529, as provided herein. In some embodiments, a ceDNA vector does not have an ITR that is selected from any sequence selected from SEQ ID NOs: 500-529.

In some embodiments, the modified ITR (e.g., the left or right ITR) of the ceDNA vector described herein has modifications within the loop arm, the truncated arm, or the spacer. Exemplary sequences of ITRs having modifications within the loop arm, the truncated arm, or the spacer are listed in Table 2 (i.e., SEQ ID NOS: 135-190, 200-233); Table 3 (e.g., SEQ ID Nos: 234-263); Table 4 (e.g., SEQ ID NOs: 264-293); Table 5 (e.g., SEQ ID Nos: 294-318 herein); Table 6 (e.g., SEQ ID NO: 319-468; and Tables 7-9 (e.g., SEQ ID Nos: 101-110, 111-112, 115-134) or Table 10A or 10B (e.g., SEQ ID Nos: 9, 100, 469-483, 484-499) of PCT application PCT/US18/49996, which is incorporated herein in its entirety by reference.

In some embodiments, the modified ITR for use in a ceDNA vector comprising an asymmetric ITR pair, or symmetric mod-ITR pair is selected from any or a combination of those shown in Tables 2, 3, 4, 5, 6, 7, 8, 9 and 10A-10B of PCT application PCT/US18/49996 which is incorporated herein in its entirety by reference.

Additional exemplary modified ITRs for use in a ceDNA vector comprising an asymmetric ITR pair, or symmetric mod-ITR pair in each of the above classes are provided in Tables 4A and 4B. The predicted secondary structure of the Right modified ITRs in Table 4A are shown in FIG. 7A, and the predicted secondary structure of the Left modified ITRs in Table 4B are shown in FIG. 7B.

Table 4A and Table 4B show exemplary right and left modified ITRs.

Table 4A: Exemplary modified right ITRs. These exemplary modified right ITRs can comprise the RBE of GCGCGCTCGCTCGCTC-3′ (SEQ ID NO: 531), spacer of ACTGAGGC (SEQ ID NO: 532), the spacer complement GCCTCAGT (SEQ ID NO: 535) and RBE′ (i.e., complement to RBE) of GAGCGAGCGAGCGCGC (SEQ ID NO: 536).

TABLE 4A
Exemplary Right modified ITRs

| | | SEQ |
|---|---|---|
| ITR | | ID |
| Construct | SEQUENCE | NO: |
| ITR-18 | ACGAACCCCTAGTGATGCACTTGGCCACTC | 469 |
| Right | CCTCTCTGCGCGCTCCCTCGCTCACTGAGG | |
| | CGCACGCCCGGGTTTCCCGGGCGGCCTCAG | |
| | TGAGCGAGCGAGCGCGCAGCTGCCTGCAGG | |
| ITR-19 | AGGAACCCCTAGTGATGGAGTTGGCCACTC | 470 |
| Right | CCTCTCTGCGCGCTCGCTCGCTCACTGAGG | |
| | CCGACGCCCGGGCTTTGCCCGGGCGGCCTC | |
| | AGTGAGCGAGCGAGCGCGCAGCTGCCTGCA | |
| | GG | |
| ITR-20 | AGGAACCCCTAGTGATGGAGTTGGCCACTC | 471 |
| Righl | CCTCTCTGCGCGCTCGCTCGCTCACTGAGG | |
| | CCGGGCGACCAAAGGTCGCCCGACGCCCGG | |
| | GCGCCTCAGTGAGCGAGCGAGCGCGCAGCT | |
| | GCCTGCAGG | |
| ITR-21 | AGGAACCCCTAGTGATGGAGTTGGCCACTC | 472 |
| Right | CCTCTCTGCGCGCTCGCTCGCTCACTGAGG | |
| | CTTTGCCTCAGTGAGCGAGCGAGCGCGCAG | |
| | CTGCCTGCAGG | |
| ITR-22 | AGGAACCCCTAGTGATGGAGTTGGCCACTC | 473 |
| Right | CCTCTCTGCGCGCTCGCTCGCTCACTGAGG | |
| | CCGGGCGACAAAGTCGCCCGACGCCCGGGC | |
| | TTTGCCCGGGCGGCCTCAGTGAGCGAGCGA | |
| | GCGCGCAGCTGCCTGCAGG | |
| ITR-23 | AGGAACCCCTAGTGATGGAGTTGGCCACTC | 474 |
| Right | CCTCTCTGCGCGCTCGCTCGCTCACTGAGG | |
| | CCGGGCGAAAATCGCCCGACGCCCGGGCTT | |
| | TGCCCGGGCGGCCTCAGTGAGCGAGCGAGC | |
| | GCGCAGCTGCCTGCAGG | |
| ITR-24 | AGGAACCCCTAGTGATGGAGTTGGCCACTC | 475 |
| Right | CCTCTCTGCGCGCTCGCTCGCTCACTGAGG | |
| | CCGGGCGAAACGCCCGACGCCCGGGCTTTG | |
| | CCCGGGCGGCCTCAGTGAGCGAGCGAGCGC | |
| | GCAGCTGCCTGCAGG | |
| ITR-25 | AGGAACCCCTAGTGATGGAGTTGGCCACTC | 476 |
| Right | CCTCTCTGCGCGCTCGCTCGCTCACTGAGG | |
| | CCCIGGCAAAGCCCGACGCCCGGGCTTTGC | |
| | CCGGGCGGCCTCAGTGAGCGAGCGAGCGCG | |
| | CAGCTGCCTGCAGG | |
| ITR-26 | AGGAACCCCTAGTGATGGAGTTGGCCACTC | 477 |
| Right | CCTCTCTGCGCGCTCGCTCGCTCACTGAGG | |
| | CCGGGCGACCAAAGGTCGCCCGACGCCCGG | |
| | GTTTCCCGGGCGGCCTCAGTGAGCGAGCGA | |
| | GCGCGCAGCTGCCTGCAGG | |
| ITR-27 | AGGAACCCCTAGTGATGGAGTTGGCCACTC | 478 |
| Right | CCTCTCTGCGCGCTCGCTCGCTCACTGAGG | |
| | CCGGGCGACCAAAGGTCGCCCGACGCCCGG | |
| | TTTCCGGGCGGCCTCAGTGAGCGAGCGAGC | |
| | GCGCAGCTGCCTGCAGG | |
| ITR-28 | AGGAACCCCTAGTGATGGAGTTGGCCACTC | 479 |
| Righl | CCTCTCTGCGCGCTCGCTCGCTCACTGAGG | |
| | CCGGGCGACCAAAGGTCGCCCGACGCCCGT | |
| | TTCGGGCGGCCTCAGTGAGCGAGCGAGCGC | |
| | GCAGCTGCCTGCAGG | |
| ITR-29 | AGGAACCCCTAGTGATGGAGTTGGCCACTC | 480 |
| Right | CCTCTCTGCGCGCTCGCTCGCTCACTGAGG | |
| | CCGGGCGACCAAAGGTCGCCCGACGCCCTT | |
| | TGGGCGGCCTCAGTGAGCGAGCGAGCGCGC | |
| | AGCTGCCTGCAGG | |
| ITR-30 | AGGAACCCCTAGTGATGGAGTTGGCCACTC | 481 |
| Righl | CCTCTCTGCGCGCTCGCTCGCTCACTGAGG | |
| | CCGGGCGACCAAAGGTCGCCCGACGCCTTT | |
| | GGCGGCCTCAGTGAGCGAGCGAGCGCGCAG | |
| | CTGCCTGCAGG | |
| ITR-31 | AGGAACCCCTAGTGATGGAGTTGGCCACTC | 482 |
| Right | CCTCTCTGCGCGCTCGCTCGCTCACTGAGG | |
| | CCGGGCGACCAAAGGTCGCCCGACGCTTTG | |
| | CGGCCTCAGTGAGCGAGCGAGCGCGCAGCT | |
| | GCCTGCAGG | |
| ITR-32 | AGGAACCCCTAGTGATGGAGTTGGCCACTC | 483 |
| Right | CCTCTCTGCGCGCTCGCTCGCTCACTGAGG | |
| | CCGGGCGACCAAAGGTCGCCCGACGTTTCG | |
| | GCCTCAGTGAGCGAGCGAGCGCGCAGCTGC | |
| | CTGCAGG | |
| ITR-49 | AGGAACCCCTAGTGATGGAGTTGGCCACTC | 99 |
| Right | CCTCTCTGCGCGCTCGCTCGCTCACTGAGG | |
| | CCGGGCGACCAAAGGTCGCCCGACGGCCTC | |
| | AGTGAGCGAGCGAGCGCGCAGCTGCCTGCA | |
| | GG | |
| ITR-50 | AGGAACCCCTAGTGATGGAGTTGGCCACTC | 100 |
| right | CCTCTCTGCGCGCTCGCTCGCTCACTGAGG | |
| | CCGGGCGACCAAAGGTCGCCCGACGCCCGG | |
| | GCGGCCTCAGTGAGCGAGCGAGCGCGCAGC | |
| | TGCCTGCAGG | |

TABLE 4B: Exemplary modified left ITRs. These exemplary modified left ITRs can comprise the RBE of GCGCGCTCGCTCGCTC-3′ (SEQ ID NO: 531), spacer of ACTGAGGC (SEQ ID NO: 532), the spacer complement GCCTCAGT (SEQ ID NO: 535) and RBE complement (RBE′) of GAGCGAGCGAGCGCGC (SEQ ID NO: 536).

TABLE 14B
Exemplary modified left ITRs

| | | |
|---|---|---|
| ITR-33 | CCTGCAGGCAGCTGCGCGCTCGCTC | 484 |
| Left | GCTCACTGAGGCCGCCCGGGAAACC | |
| | CGGGCGTGCGCCTCAGTGAGCGAGC | |
| | GAGCGCGCAGAGAGGGAGTGGCCAA | |
| | CTCCATCACTAGGGGTTCCT | |
| ITR-34 | CCTGCAGGCAGCTGCGCGCTCGCTC | 485 |
| Left | GCTCACTGAGGCCGTCGGGCGACCT | |
| | TTGGTCGCCCGGCCTCAGTGAGCGA | |
| | GCGAGCGCGCAGAGAGGGAGTGGCC | |
| | AACTCCATCACTAGGGGTTCCT | |
| ITR-35 | CCTGCAGGCAGCTGCGCGCTCGCTC | 486 |
| Left | GCTCACTGAGGCCGCCCGGGCAAAG | |
| | CCCGGGCGTCGGCCTCAGTGAGCGA | |
| | GCGAGCGCGCAGAGAGGGAGTGGCC | |
| | AACTCCATCACTAGGGGTTCCT | |
| ITR-36 | CCTGCAGGCAGCTGCGCGCTCGCTC | 487 |
| Left | GCTCACTGAGGCGCCCGGGCGTCGG | |
| | GCGACCTTTGGTCGCCCGGCCTCAG | |
| | TGAGCGAGCGAGCGCGCAGAGAGGG | |
| | AGTGGCCAACTCCATCACTAGGGGT | |
| | TCCT | |
| ITR-37 | CCTGCAGGCAGCTGCGCGCTCGCTC | 488 |
| Left | GCTCACTGAGGCAAAGCCTCAGTGA | |
| | GCGAGCGAGCGCGCAGAGAGGGAGT | |
| | GGCCAACTCCATCACTAGGGGTTCC | |
| | T | |
| ITR-38 | CCTGCAGGCAGCTGCGCGCTCGCTC | 489 |
| Left | GCTCACTGAGGCCGCCCGGGCAAAG | |
| | CCCGGGCGTCGGGCGACTTTGTCGC | |
| | CCGGCCTCAGTGAGCGAGCGAGCGC | |
| | GCAGAGAGGGAGTGGCCAACTCCAT | |
| | CACTAGGGGTTCCT | |
| ITR-39 | CCTGCAGGCAGCTGCGCGCTCGCTC | 490 |
| Left | GCTCACTGAGGCCGCCCGGGCAAAG | |
| | CCCGGGCGTCGGGCGATTTTCGCCC | |
| | GGCCTCAGTGA | |
| | GCGAGCGAGCGCGCAGAGAGGGAGT | |
| | GGCCAACTCCATCACTAGGGGTTCC | |
| | T | |
| ITR-40 | CCTGCAGGCAGCTGCGCGCTCGCTC | 491 |
| Left | GCTCACTGAGGCCGCCCGGGCAAAG | |
| | CCCGGGCGTCGGGCGTTTCGCCCGG | |
| | CCTCAGTGAGCGAGCGAGCGCGCAG | |
| | AGAGGGAGTGGCCAACTCCATCACT | |
| | AGGGGTTCCT | |
| ITR-4I | CCTGCAGGCAGCTGCGCGCTCGCTC | 492 |
| Left | GCTCACTGAGGCCGCCCGGGCAAAG | |
| | CCCGGGCGTCGGGCTTTGCCCGGCC | |
| | TCAGTGAGCGAGCGAGCGCGCAGAG | |
| | AGGGAGTGGCCAACTCCATCACTAG | |
| | GGGTTCCT | |
| ITR-42 | CCTGCAGGCAGCTGCGCGCTCGCTC | 493 |
| Left | GCTCACTGAGGCCGCCCGGGAAACC | |
| | CGGGCGTCGGGCGACCTTTGGTCGC | |
| | CCGGCCTCAGTGAGCGAGCGAGCGC | |
| | GCAGAGAGGGAGTGGCCAACTCCAT | |
| | CACTAGGGGTTCCT | |
| ITR-43 | CCTGCAGGCAGCTGCGCGCTCGCTC | 494 |
| Left | GCTCACTGAGGCCGCCCGOAAACCG | |
| | GGCGTCGGGCGACCTTTGGTCGCCC | |
| | GGCCTCAGTGAGCGAGCGAGCGCGC | |
| | AGAGAGGGAGTGGCCAACTCCATCA | |
| | CTAGGGGTTCCT | |
| ITR-44 | CCTGCAGGCAGCTGCGCGCTCGCTC | 495 |
| Left | GCTCACTGAGGCCGCCCGAAACGGG | |
| | CGTCGGGCGACCTTTGGTCGCCCGG | |
| | CCTCAGTGAGCGAGCGAGCGCGCAG | |
| | AGAGGGAGTGGCCAACTCCATCACT | |
| | AGGGGTTCCT | |
| ITR-45 | CCTGCAGGCAGCTGCGCGCTCGCTC | 496 |
| Left | GCTCACTGAGGCCGCCCAAAGGGCG | |
| | TCGGGCGACCTTTGGTCGCCCGGCC | |
| | TCAGTGAGCGAGCGAGCGCGCAGAG | |
| | AGGGAGTGGCCAACTCCATCACTAG | |
| | GGGTTCCT | |
| ITR-46 | CCTGCAGGCAGCTGCGCGCTCGCTC | 497 |
| Left | GCTCACTGAGGCCGCCAAAGGCGTC | |
| | GGGCGACCTTTGGTCGCCCGGCCTC | |
| | AGTGAGCGAGCGAGCGCGCAGAGAG | |
| | GGAGTGGCCAACTCCATCACTAGGG | |
| | GTTCCT | |
| ITR-47 | CCTGCAGGCAGCTGCGCGCTCGCTC | 498 |
| Left | GCTCACTGAGGCCGCAAAGCGTCGG | |
| | GCGACCTTTGGTCGCCCGGCCTCAG | |
| | TGAGCGAGCGAGCGCGCAGAGAGGG | |
| | AGTGGCCAACTCCATCACTAGGGGT | |
| | TCCT | |
| ITR-48 | CCTGCAGGCAGCTGCGCGCTCGCTC | 499 |
| Left | GCTCACTGAGGCCGAAACGTCGGGC | |
| | GACCTTTGGTCGCCCGGCCTCAGTG | |
| | AGCGAGCGAGCGCGCAGAGAGGGAG | |
| | TGGCCAACTCCATCACTAGGG | |
| | GTTCCT | |

In one embodiment, a gene editing ceDNA vector comprises two symmetrical mod-ITRs—that is, both ITRs have the same sequence, but are reverse complements (inverted) of each other. In some embodiments, a symmetrical mod-ITR pair comprises at least one or any combination of a deletion, insertion, or substitution relative to wild type ITR sequence from the same AAV serotype. The additions, deletions, or substitutions in the symmetrical ITR are the same but the reverse complement of each other. For example, an insertion of 3 nucleotides in the C region of the 5′ ITR would be reflected in the insertion of 3 reverse complement nucleotides in the corresponding section in the C′ region of the 3′ ITR. Solely for illustration purposes only, if the addition is AACG in the 5′ ITR, the addition is CGTT in the 3′ ITR at the corresponding site. For example, if the 5′ ITR sense strand is ATCGATCG with an addition of AACG between the G and A to result in the sequence ATCGAACGATCG. The corresponding 3′ ITR sense strand is CGATCGAT (the reverse complement of ATCGATCG) with an addition of CGTT (i.e. the reverse complement of AACG) between the T and C to result in the sequence CGATCGTTCGAT (the reverse complement of ATCGAACGATCG).

In alternative embodiments, the modified ITR pair are substantially symmetrical as defined herein—that is, the modified ITR pair can have a different sequence but have corresponding or the same symmetrical three-dimensional shape. For example, one modified ITR can be from one serotype and the other modified ITR be from a different serotype, but they have the same mutation (e.g., nucleotide insertion, deletion or substitution) in the same region. Stated differently, for illustrative purposes only, a 5′ mod-ITR can be from AAV2 and have a deletion in the C region, and the 3′ mod-ITR can be from AAVS and have the corresponding deletion in the C′ region, and provided the 5′ mod-ITR and the 3′ mod-ITR have the same or symmetrical three-dimensional spatial organization, they are encompassed for use herein as a modified ITR pair.

In some embodiments, a substantially symmetrical mod-ITR pair has the same A, C-C′ and B-B′ loops in 3D space, e.g., if a modified ITR in a substantially symmetrical mod-ITR pair has a deletion of a C-C′ arm, then the cognate mod-ITR has the corresponding deletion of the C-C′ loop and also has a similar 3D structure of the remaining A and B-B′ loops in the same shape in geometric space of its cognate mod-ITR. By way of example only, substantially symmetrical ITRs can have a symmetrical spatial organization such that their structure is the same shape in geometrical space. This can occur, e.g., when a G-C pair is modified, for example, to a C-G pair or vice versa, or A-T pair is modified to a T-A pair, or vice versa. Therefore, using the exemplary example above of modified 5′ ITR as a ATCGAACGATCG (SEQ ID NO: 570), and modified 3′ ITR as CGATCGTTCGAT (SEQ ID NO: 571) (i.e., the reverse complement of ATCGAACGATCG (SEQ ID NO: 570)), these modified ITRs would still be symmetrical if, for example, the 5′ ITR had the sequence of ATCGAACCATCG (SEQ ID NO: 572), where G in the addition is modified to C, and the substantially symmetrical 3′ ITR has the sequence of CGATCGTTCGAT (SEQ ID NO: 571), without the corresponding modification of the T in the addition to a A. In some embodiments, such a modified ITR pair are substantially symmetrical as the modified ITR pair has symmetrical stereochemistry.

Table 5 shows exemplary symmetric modified ITR pairs (i.e. a left modified ITRs and the symmetric right modified ITR). The bold (red) portion of the sequences identify partial ITR sequences (i.e., sequences of A-A′, C-C′ and B-B′ loops), also shown in FIGS. 31A-46B. These exemplary modified ITRs can comprise the RBE of GCGCGCTCGCTCGCTC-3′ (SEQ ID NO: 531), spacer of ACTGAGGC (SEQ ID NO: 532), the spacer complement GCCTCAGT (SEQ ID NO: 535) and RBE′ (i.e., complement to RBE) of GAGCGAGCGAGCGCGC (SEQ ID NO: 536).

TABLE 5
exemplary symmetric modified ITR pairs

| LEFT modified ITR | | Symmetric RIGHT modified ITR | |
|---|---|---|---|
| (modified 5′ ITR) | | (modified 3′ ITR) | |
| SEQ ID | CCTGCAGGCAGCTGCGCGCTCGCT | SEQ ID NO: | AGGAACCCCTAGTGATGGAG |
| NO: 484 | CGCTCACTGAGGCCGCCCGGGAAA | 469 (ITR- | TTGGCCACTCCCTCTCTGCG |
| (ITR-33 | CCCGGGCGTGCGCCTCAGTGAGCG | 18, right) | CGCTCGCTCGCTCACTGAGG |
| left) | AGCGAGCGCGCAGAGAGGGAGTGG | | CGCACGCCCGGGTTTCCCGG |
| | CCAACTCCATCACTAGGGGTTCCT | | GCGGCCTCAGTGAGCGAGCG |
| | | | AGCGCGCAGCTGCCTGCAGG |
| SEQ ID | CCTGCAGGCAGCTGCGCGCTCGCT | SEQ ID NO: | AGGAACCCCTAGTGATGGAG |
| NO: 485 | CGCTCACTGAGGCCGTCGGGCGAC | 95 (ITR-51, | TTGGCCACTCCCTCTCTGCG |
| (ITR-34 | CTTTGGTCGCCCGGCCTCAGTGAG | right) | CGCTCGCTCGCTCACTGAGG |
| left) | CGAGCGAGCGCGCAGAGAGGGAGT | | CCGGGCGACCAAAGGTCGCC |
| | GGCCAACTCCATCACTAGGGGTTC | | CGACGGCCTCAGTGAGCGAG |
| | CT | | CGAGCGCGCAGCTGCCTGCA |
| | | | GG |
| SEQ ID | CCTGCAGGCAGCTGCGCGCTCGCT | SEQ ID NO: | AGGAACCCCTAGTGATGGAG |
| NO: 486 | CGCTCACTGAGGCCGCCCGGGCAA | 470 (ITR- | TTGGCCACTCCCTCTCTGCG |
| (ITR-35 | AGCCCGGGCGTCGGCCTCAGTGAG | 19, right) | CGCTCGCTCGCTCACTGAGG |
| left) | CGAGCGAGCGCGCAGAGAGGGAGT | | CCGACGCCCGGGCTTTGCCC |
| | GGCCAACTCCATCACTAGGGGTTC | | GGGCGGCCTCAGTGAGCGAG |
| | CT | | CGAGCGCGCAGCTGCCTGCA |
| | | | GG |
| SEQ ID | CCTGCAGGCAGCTGCGCGCTCGCT | SEQ ID NO: | AGGAACCCCTAGTGATGGAG |
| NO: 487 | CGCTCACTGAGGCGCCCGGGCGTC | 471 (ITR- | TTCCCCACTCCCTCTCTGCG |
| (ITR-36 | GGGCGACCTTTGGTCGCCCGGCCT | 20, right) | CGCTCGCTCGCTCACTGAGG |
| left) | CAGTGAGCGAGCGAGCGCGCAGAG | | CCGGGCGACCAAAGGTCGCC |
| | AGGGAGTGGCCAACTCCATCACTA | | CGACGCCCGGGCGCCTCAGT |
| | GGGGTTCCT | | GAGCGAGCGAGCGCGCAGCT |
| | | | GCCTGCAGG |
| SEQ ID | CCTGCAGGCAGCTGCGCGCTCGCT | SEQ ID NO: | AGGAACCCCTAGTGATGGAG |
| NO: 488 | CGCTCACTGAGGCAAAGCCTCAGT | 472 (ITR- | TTGGCCACTCCCTCTCTGCG |
| (ITR-37 | GAGCGAGCGAGCGCGCAGAGAGGG | 21, right) | CGCTCGCTCGCTCACTGAGG |
| left) | AGTGGCCAACTCCATCACTAGGGG | | CTTTGCCTCAGTGAGCGAGC |
| | TTCCT | | GAGCGCGCAGCTGCCTGCAG |
| | | | G |
| SEQ ID | CCTGCAGGCAGCTGCGCGCTCGCT | SEQ ID NO: | AGGAACCCCTAGTGATGGAG |
| NO: 489 | CGCTCACTGAGGCCGCCCGGGCAA | 473 (ITR-22 | TTGGCCACTCCCTCTCTGCG |
| (ITR-38 | AGCCCGGGCGTCGGGCGACTTTGT | right) | CGCTCGCTCGCTCACTGAGG |
| left) | CGCCCGGCCTCAGTGAGCGAGCGA | | CCGGGCGACAAAGTCGCCCG |
| | GCGCGCAGAGAGGGAGTGGCCAAC | | ACGCCCGGGCTTTGCCCGGG |
| | TCCATCACTAGGGGTTCCT | | CGGCCTCAGTGAGCGAGCGA |
| | | | GCGCGCAGCTGCCTGCAGG |
| SEQ ID | CCTGCAGGCAGCTGCGCGCTCGCT | SEQ ID NO: | AGGAACCCCTAGTGATGGAG |
| NO: 490 | CGCTCACTGAGGCCGCCCGGGCAA | 474 (ITR- | TTGGCCACTCCCTCTCTGCG |
| (ITR-39 | AGCCCGGGCGTCGGGCGATTTTCG | 23, right) | CGCTCGCTCGCTCACTGAGG |
| left) | CCCGGCCTCAGTGAGCGAGCGAGC | | CCGGGCGAAAATCGCCCGAC |
| | GCGCAGAGAGGGAGTGGCCAACTC | | GCCCGGGCTTTGCCCGGGCG |
| | CATCACTAGGGGTTCCT | | GCCTCAGTGAGCGAGCGAGC |
| | | | GCGCAGCTGCCTGCAGG |
| SEQ ID | CCTGCAGGCAGCTGCGCGCTCGCT | SEQ ID NO: | AGGAACCCCTAGTGATGGAG |
| NO: 491 | CGCTCACTGAGGCCGCCCGGGCAA | 475 (ITR- | TTGGCCACTCCCTCTCTGCG |
| (ITR-40 | AGCCCGGGCGTCGGGCGTTTCGCC | 24, right) | CGCTCGCTCGCTCACTGAGG |
| left) | CGGCCTCAGTGAGCGAGCGAGCGC | | CCGGGCGAAACGCCCGACGC |
| | GCAGAGAGGGAGTGGCCAACTCCA | | CCGGGCTTTGCCCGGGCGGC |
| | TCACTAGGGGTTCCT | | CTCAGTGAGCGAGCGAGCGC |
| | | | GCAGCTGCCTGCAGG |
| SEQ ID | CCTGCAGGCAGCTGCGCGCTCGCT | SEQ ID NO: | AGGAACCCCTAGTGATGGAG |
| NO: 492 | CGCTCACTGAGGCCGCCCGGGCAA | 476 (ITR-25 | TTCCCCACTCCCTCTCTGCG |
| (ITR-41 | AGCCCGGGCGTCGGGCTTTGCCCG | right) | CGCTCGCTCGCTCACTGAGG |
| left) | GCCTCAGTGAGCGAGCGAGCGCGC | | CCGGGCAAAGCCCGACGCCC |
| | AGAGAGGGAGTGGCCAACTCCATC | | GGGCTTTGCCCGGGCGGCCT |
| | ACTAGGGGTTCCT | | CAGTGAGCGAGCGAGCGCGC |
| | | | AGCTGCCTGCAGG |
| SEQ ID | CCTGCAGGCAGCTGCGCGCTCGCT | SEQ ID NO: | AGGAACCCCTAGTGATGGAG |
| NO: 493 | CGCTCACTGAGGCCGCCCGGGAAA | 477 (ITR-26 | TTGGCCACTCCCTCTCTGCG |
| (ITR-42 | CCCGGGCGTCGGGCGACCTTTGGT | right) | CGCTCGCTCGCTCACTGAGG |
| left) | CGCCCGGCCTCAGTGAGCGAGCGA | | CCGGGCGACCAAAGGTCGCC |
| | GCGCGCAGAGAGGGAGTGGCCAAC | | CGACGCCCGGGTTTCCCGGG |
| | TCCATCACTAGGGGTTCCT | | CGGCCTCAGTGAGCGAGCGA |
| | | | GCGCGCAGCTGCCTGCAGG |
| SEQ ID | CCTGCAGGCAGCTGCGCGCTCGCT | SEQ ID NO: | AGGAACCCCTAGTGATGGAG |
| NO: 494 | CGCTCACTGAGGCCGCCCGGAAAC | 478 (ITR-27 | TTGGCCACTCCCTCTCTGCG |
| (ITR-43 | CGGGCGTCGGGCGACCTTTGGTCG | right) | CGCTCGCTCGCTCACTGAGG |
| left) | CCCGGCCTCAGTGAGCGAGCGAGC | | CCGGGCGACCAAAGGTCGCC |
| | GCGCAGAGAGGGAGTGGCCAACTC | | CGACGCCCGGTTTCCGGGCG |
| | CATCACTAGGGGTTCCT | | GCCTCAGTGAGCGAGCGAGC |
| | | | GCGCAGCTGCCTGCAGG |
| SEQ ID | CCTGCAGGCAGCTGCGCGCTCGCT | SEQ ID NO: | AGGAACCCCTAGTGATGGAG |
| NO: 495 | CGCTCACTGAGGCCGCCCGAAACG | 479 (ITR-28 | TTGGCCACTCCCTCTCTGCG |
| (ITR-44 | GGCGTCGGGCGACCTTTGGTCGCC | right) | CGCTCGCTCGCTCACTGAGG |
| left) | CGGCCTCAGTGAGCGAGCGAGCGC | | CCGGGCGACCAAAGGTCGCC |
| | GCAGAGAGGGAGTGGCCAACTCCA | | CGACGCCCGTTTCGGGCGGC |
| | TCACTAGGGGTTCCT | | CTCAGTGAGCGAGCGAGCGC |
| | | | GCAGCTGCCTGCAGG |
| SEQ ID | CCTGCAGGCAGCTGCGCGCTCGCT | SEQ ID | AGGAACCCCTAGTGATGGAG |
| NO: 496 | CGCTCACTGAGGCCGCCCAAAGGG | NO: 480 | TTGGCCACTCCCTCTCTGCG |
| (ITR-45 | CGTCGGGCGACCTTTGGTCGCCCG | (ITR-29, | CGCTCGCTCGCTCACTGAGG |
| left) | GCCTCAGTGAGCGAGCGAGCGCGC | right) | CCGGGCGACCAAAGGTCGCC |
| | AGAGAGGGAGTGGCCAACTCCATC | | CGACGCCCTTTGGGCGGCCT |
| | ACTAGGGGTTCCT | | CAGTGAGCGAGCGAGCGCGC |
| | | | AGCTGCCTGCAGG |
| SEQ ID | CCTGCAGGCAGCTGCGCGCTCGCT | SEQ ID NO: | AGGAACCCCTAGTGATGGAG |
| NO: 497 | CGCTCACTGAGGCCGCCAAAGGCG | 481 (ITR- | TTCCCCACTCCCTCTCTGCG |
| (ITR-46 | TCGGGCGACCTTTGGTCGCCCGGC | 30, right) | CGCTCGCTCGCTCACTGAGG |
| left) | CTCAGTGAGCGAGCGAGCGCGCAG | | CCGGGCGACCAAAGGTCGCC |
| | AGAGGGAGTGGCCAACTCCATCAC | | CGACGCCTTTGGCGGCCTCA |
| | TAGGGGTTCCT | | GTGAGCGAGCGAGCGCGCAG |
| | | | CTGCCTGCAGG |
| SEQ ID | CCTGCAGGCAGCTGCGCGCTCGCT | SEQ ID NO: | AGGAACCCCTAGTGATGGAG |
| NO: 498 | CGCTCACTGAGGCCGCAAAGCGTC | 482 (ITR- | TTGGCCACTCCCTCTCTGCG |
| (ITR- | GGGCGACCTTTGGTCGCCCGGCCT | 31, right) | CGCTCGCTCGCTCACTGAGG |
| 47, | CAGTGAGCGAGCGAGCGCGCAGAG | | CCGGGCGACCAAAGGTCGCC |
| left) | AGGGAGTGGCCAACTCCATCACTA | | CGACGCTTTGCGGCCTCAGT |
| | GGGGTTCCT | | GAGCGAGCGAGCGCGCAGCT |
| | | | GCCTGCAGG |
| SEQ ID | CCTGCAGGCAGCTGCGCGCTCGCT | SEQ ID NO: | AGGAACCCCTAGTGATGGAG |
| NO: 499 | CGCTCACTGAGGCCGAAACGTCGG | 483 (ITR-32 | TTGGCCACTCCCTCTCTGCG |
| (ITR- | GCGACCTTTGGTCGCCCGGCCTCA | right) | CGCTCGCTCGCTCACTGAGG |
| 48, | GTGAGCGAGCGAGCGCGCAGAGAG | | CCGGGCGACCAAAGGTCGCC |
| left) | GGAGTGGCCAACTCCATCACTAGG | | CGACGTTTCGGCCTCAGTGA |
| | GGTTCCT | | GCGAGCGAGCGCGCAGCTGC |
| | | | CTGCAGG |

In some embodiments, a ceDNA vector for gene editing comprising an asymmetric ITR pair can comprise an ITR with a modification corresponding to any of the modifications in ITR sequences or ITR partial sequences shown in any one or more of Tables 4A-4B herein or the sequences shown in FIG. 7A or 7B, or disclosed in Tables 2, 3, 4, 5, 6, 7, 8, 9 or 10A-10B of PCT/US18/49996 filed Sep. 7, 2018 which is incorporated herein in its entirety by reference.

### VI. Exemplary Gene Editing ceDNA Vectors

As described above, the present disclosure relates to recombinant ceDNA expression vectors (e.g., donor vectors (may or may not be operably linked to a promoter) and ceDNA vectors that encode gene editing molecules) comprising any one of: an asymmetrical ITR pair, a symmetrical ITR pair, or substantially symmetrical ITR pair as described above. In certain embodiments, the disclosure relates to recombinant ceDNA vectors having flanking ITR sequences and gene editing capabilities, where the ITR sequences are asymmetrical, symmetrical or substantially symmetrical relative to each other as defined herein, and the ceDNA further comprises a nucleotide sequence of interest (for example an expression cassette of a gene editing sequence, or a guide RNA) located between the flanking ITRs, wherein said nucleic acid molecule is devoid of viral capsid protein coding sequences.

In some embodiments the ceDNA vector encompasses at least one of the following: a nuclease, one or more homology arms, a guide RNA, an activator RNA, and a control element. In some embodiments, a polynucleotide including a 5′ homology arm, a donor sequence, and a 3′ homology arm. Suitable ceDNA vectors in accordance with the present disclosure may be obtained by following the Examples below. In certain embodiments, the disclosure relates to recombinant ceDNA expression vectors comprising at least two components of a gene editing system, e.g. CAS and at least one gRNA, or two ZNFs, etc. Thus, in some embodiments, the ceDNA vectors comprise multiple components of a gene editing system.

The recombinant ceDNA expression vector may be any ceDNA vector that can be conveniently subjected to recombinant DNA procedures including nucleotide sequence(s) as described herein, provided at least one ITR is altered. The ceDNA vectors of the present disclosure are compatible with the host cell into which the ceDNA vector is to be introduced. In certain embodiments, the ceDNA vectors may be linear. In certain embodiments, the ceDNA vectors may exist as an extrachromosomal entity. In certain embodiments, the ceDNA vectors of the present disclosure may contain an element(s) that permits integration of a donor sequence into the host cell's genome. As used herein “donor sequence” and “transgene” and “heterologous nucleotide sequence” are synonymous.

Referring now to FIGS. 1A-1G, schematics of the functional components of two non-limiting plasmids useful in making the ceDNA vectors of the present disclosure are shown. FIG. 1A, 1B, 1F show the construct of ceDNA vectors for gene editing or the corresponding sequences of ceDNA plasmids. ceDNA vectors are capsid-free and can be obtained from a plasmid encoding in this order: a first ITR, an expressible transgene cassette and a second ITR, where the first and second ITR sequences are asymmetrical, symmetrical or substantially symmetrical relative to each other as defined herein. ceDNA vectors are capsid-free and can be obtained from a plasmid encoding in this order: a first ITR, an expressible transgene (protein or nucleic acid) or donor cassette (e.g. HDR donor) and a second ITR, where the first and second ITR sequences are asymmetrical, symmetrical or substantially symmetrical relative to each other as defined herein. In some embodiments, the expressible transgene cassette includes, as needed: an enhancer/promoter, one or more homology arms, a donor sequence, a post-transcription regulatory element (e.g., WPRE, e.g., SEQ ID NO: 8)), and a polyadenylation and termination signal (e.g., BGH polyA, e.g., SEQ ID NO: 7).

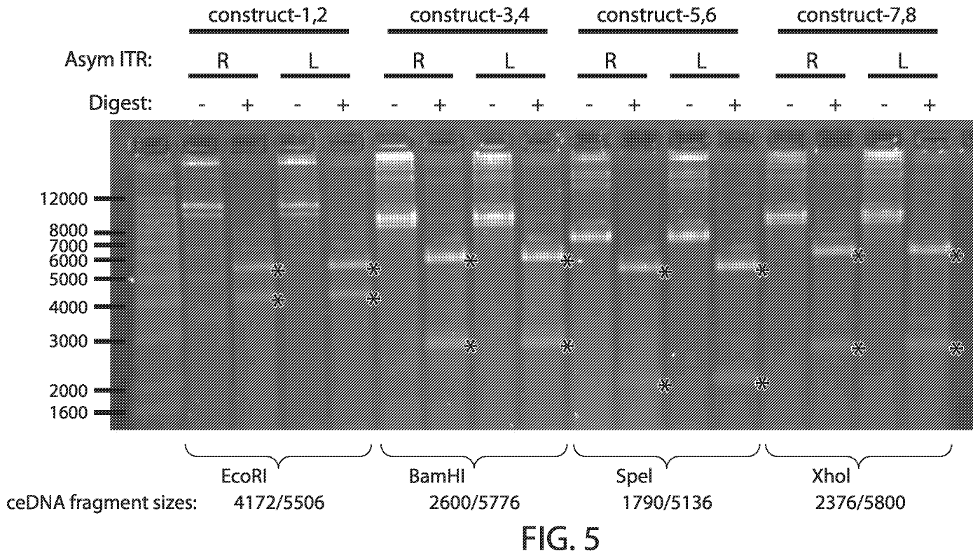

FIG. 5 is a gel confirming the production of ceDNA from multiple plasmid constructs using the method described in the Examples. The ceDNA is confirmed by a characteristic band pattern in the gel, as discussed with respect to FIG. 4A above and in the Examples.

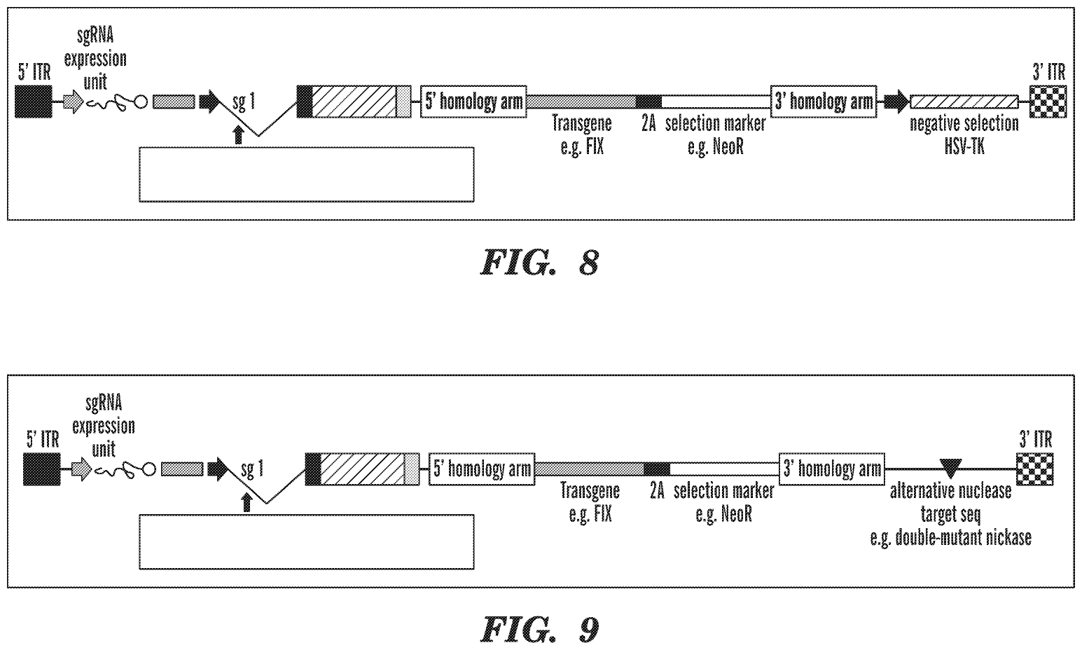

Referring now to FIG. 8, a nonlimiting exemplary ceDNA vector in accordance with the present disclosure is shown including a first and second ITR, where the ITR sequences are asymmetrical, symmetrical or substantially symmetrical relative to each other as defined herein, a first nucleotide sequence including a 5′ homology arm, a donor sequence, and a 3′ homology arm, wherein the donor sequence has gene editing functionality. In some embodiments, TRs (e.g. ITRs) as described above are included on the flanking ends of the nucleic acid sequence encoding a gene editing molecule of interest (e.g., a nuclease (e.g., sequence specific nuclease), one or more guide RNA, Cas or other ribonucleoprotein (RNP), or any combination thereof. Non-limiting examples of the nucleic acid constructs of the present disclosure include a nucleic acid construct including a wild-type functioning ITR of AAV2 having the nucleotide sequence of SEQ ID NO:1, or SEQ ID NO:51 and further an altered ITR of AAV2 having at least 60%, more preferably at least 65%, more preferably at least 70%, more preferably at least 75%, more preferably at least 80%, more preferably at least 85%, even more preferably at least 90%, and most preferably at least 95% sequence identity to the nucleotide sequence of SEQ ID NO: 2 or SEQ ID NO: 52. Additional ITRs are described in WO 2017/152149 and PCT application PCT/US18/49996, herein incorporated by reference in their entirety.

Referring to FIG. 8, a ceDNA can comprise a second nucleotide sequence upstream of the first nucleotide sequence as shown. In certain embodiments of any of the ceDNA vectors described herein, the ceDNA vector can further comprise such a second nucleotide sequence 5′ or 3′ of the first nucleotide sequence comprising a donor sequence and, optionally, homology arms. In some embodiments, referring to FIG. 8, the ceDNA vector may include a third nucleotide sequence including a second promoter operably linked to the one or more nucleotides encoding the guide sequence and/or activator RNA sequence. In certain embodiments, the promoter is Pol III (U6 (SEQ ID NO:18), or H1 (SEQ ID NO: 19)).

In another embodiment, a ceDNA vector encodes a nuclease and one or more guide RNAs that are directed to each of the ceDNA ITRs, or directed to outside the Homology domain regions, for torsional release and more efficient homoloy directed repair (HDR). The nuclease need not be a mutant nuclease, e.g. the donor HDR template may be released from ceDNA by such cleavage.

In some embodiments, in one nonlimiting example, a ceDNA vector for gene editing can comprise a 5′ and 3′ homology arm to a specific gene, or target intergration site that has restriction sites specific for an endonuclease described herein at either end of the 5′ homology and 3′ homology arm. When the ceDNA vector is cleaved with the one or more restriction endonucleases specific for the restriction site(s), the resulting cassette comprises the 5′ homology arm-donor sequence-3′ homology arm, and can be more readily recombined with the desired genomic locus. In certain aspects, the ceDNA vector itself may encode the restriction endonuclease such that upon delivery of the ceDNA vector to the nucleus, the restriction endonuclease is expressed and able to cleave the vector. In certain aspects, the restriction endonuclease is encoded on a second ceDNA vector which is separately delivered. In certain aspects, the restriction endonuclease is introduced to the nucleus by a non-ceDNA-based means of delivery. Accordingly, in some embodiments, the technology described herein enables more than one gene editing ceDNA being delivered to a subject. As discussed herein, in one embodiment, a ceDNA can have the homology arms flanking a donor sequence that targets a specific target gene or locus, and can in some embodiments, also include one or more guide RNAs (e.g., sgRNA) for targeting the cutting of the genomic DNA, as described herein, and another ceDNA can comprise a nuclease enzyme and activator RNA, as described herein for the actual gene editing steps.

A. DNA Endonucleases

The ceDNA vectors of the present disclosure may contain a nucleotide sequence that encodes a nuclease, such as a sequence-specific nuclease. Sequence-specific or site-specific nucleases can be used to introduce site-specific double strand breaks or nicks at targeted genomic loci. This nucleotide cleavage, e.g., DNA or RNA cleavage, stimulates the natural repair machinery, e.g., DNA repair machinery, leading to one of two possible repair pathways. In the absence of a donor template, the break will be repaired by non-homologous end joining (NHEJ), an error-prone repair pathway that leads to small insertions or deletions of DNA (see e.g., Suzuki et al. Nature 540:144-149 (2016), the contents of which are incorporated by reference in its entirety). This method can be used to intentionally disrupt, delete, or alter the reading frame of targeted gene sequences. However, if a donor template is provided in addition to the nuclease, then the cellular machinery will repair the break by homologous recombination (HDR), which is enhanced several orders of magnitude in the presence of DNA cleavage, or by insertion of the donor template via NHEJ.

The methods can be used to introduce specific changes in the DNA sequence at target sites. The term “site-specific nuclease” as used herein refers to an enzyme capable of specifically recognizing and cleaving a particular DNA sequence. The site-specific nuclease may be engineered. Examples of engineered site-specific nucleases include zinc finger nucleases (ZFNs), TAL effector nucleases (TALENs), meganucleases, and CRISPR/Cas9-enzymes and engineered derivatives. As will be appreciated by those of skill in the art, the endonucleases necessary for gene editing can be expressed transiently, as there is generally no further need for the endonuclease once gene editing is complete. Such transient expression can reduce the potential for off-target effects and immunogenicity. Transient expression can be accomplished by any known means in the art, and may be conveniently effected using a regulatory switch as described herein.

In some embodiments, the nucleotide sequence encoding the nuclease is cDNA. Non-limiting examples of sequence-specific nucleases include RNA-guided nuclease, zinc finger nuclease (ZFN), a transcription activator-like effector nuclease (TALEN) or a meganuclease. Non-limiting examples of suitable RNA-guided nucleases include CRISPR enzymes as described herein.

The nucleases described herein can be altered, e.g., engineered to design sequence specific nuclease (see e.g., U.S. Pat. No. 8,021,867). Nucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514, the contents of each are incorporated herein by reference in their entirety. Alternatively, nuclease with site specific cutting characteristics can be obtained using commercially available technologies e.g., Precision BioSciences' Directed Nuclease Editor™ genome editing technology.

In certain embodiments, for example when using a promoterless ceDNA construct comprising a homology directed repair template, the guide RNA and/or Cas enzyme, or any other nuclease, are delivered in trans, e.g. by administering i) a nucleic acid encoding a guide RNA, ii) or an mRNA encoding a the desired nuclease, e.g. Cas enzyme, or other nuclease iii) or by administering a ribonucleotide protein (RNP) complex comprising a Cas enzyme and a guide RNA, or iv) e.g., delivery of recombinant nuclease proteins by vector, e.g. viral, plasmid, or another ceDNA vector. In certain aspects, the molecules delivered in trans are delivered by means of one or more additional ceDNA vectors which can be co-administered or administered sequentially to the first ceDNA vector.

Accordingly, in one embodiment, a ceDNA vector can comprise an endonuclease (e.g., Cas9) that is transcriptionally regulated by an inducible promoter. In some embodiments, the endonuclease is on a separate ceDNA vector, which can be administered to a subject with a ceDNA comprising homology arms and a donor sequence, which can optionally also comprise guide RNA (sgRNAs). In alternative embodiments, the endonuclease can be on an all-in-one ceDNA vector as described herein.

In some embodiments, the ceDNA encodes an endonuclease as described herein under control of a promoter. Non-limiting examples of inducible promoters include chemically-regulated promoters, which regulate transcriptional activity by the presence or absence of, for example, alcohols, tetracycline, steroids, metal, and pathogenesis-related proteins (e.g., salicylic acid, ethylene, and benzothiadiazole), and physically-regulated promoters, which regulate transcriptional activity by, for example, the presence or absence of light and low or high temperatures. Modulation of the inducible promoter allows for the turning off or on of gene-editing activity of a ceDNA vector. Inducible Cas9 promoters are further reviewed, for example in Cao J., et al. Nucleic Acids Research. 44(19)2016, and Liu K I, et al. Nature Chemical Biol. 12: 90-987 (2016), which are incorporated herein in their entireties.

In one embodiment, the ceDNA vector described herein further comprises a second endonuclease that temporally targets and inhibits the activity of the first endonuclease (e.g., Cas9). Endonucleases that target and inhibit the activity of other endonucleases can be determined by those skilled in the art. In another embodiment, the ceDNA vector described herein further comprises temporal expression of an “anti-CRISPR gene” (e.g., L. monocytogenes ArcIIa). As used herein, “anti-CRISPR gene” refers to a gene shown to inhibit the commonly used S. pyogenes Cas9. In another embodiment, the second endonuclease that targets and inhibits the activity of the first endonuclease activity, or the anti-CRISPR gene, is comprised in a second ceDNA vector that is administered after the desired gene-editing is complete. Alternatively, the second endonuclease targets and inhibits a gene of interest, for example, a gene that has been transcriptionally enhanced by a ceDNA vector as described herein.

A ceDNA vector or composition thereof, as described herein, can include a nucleotide sequence encoding a transcriptional activator that activates a target gene. For example, the transcriptional activator may be engineered. For example, an engineered transcriptional activator may be a CRISPR/Cas9-based system, a zinc finger fusion protein, or a TALE fusion protein. The CRISPR/Cas9-based system, as described above, may be used to activate transcription of a target gene with RNA. The CRISPR/Cas9-based system may include a fusion protein, as described above, wherein the second polypeptide domain has transcription activation activity or histone modification activity. For example, the second polypeptide domain may include VP64 or p300. Alternatively, the transcriptional activator may be a zinc finger fusion protein. The zinc finger targeted DNA-binding domains, as described above, can be combined with a domain that has transcription activation activity or histone modification activity. For example, the domain may include VP64 or p300. TALE fusion proteins may be used to activate transcription of a target gene. The TALE fusion protein may include a TALE DNA-binding domain and a domain that has transcription activation activity or histone modification activity. For example, the domain may include VP64 or p300.

Another method for modulating gene expression at the transcription level is by targeting epigenetic modifications using modified DNA endonucleases as described herein. Modulation of gene expression at the epigenetic level has the advantage of being inherited by daughter cells at a higher rate than the activation/inhibition achieved using CRISPRa or CRISPRi. In one embodiment, dCas9 fused to a catalytic domain of p300 acetyltransferase can be used with the methods and compositions described herein to make epigenetic modifications (e.g., increase histone modification) to a desired region of the genome. Epigenetic modifications can also be achieved using modified TALEN constructs, such as a fusion of a TALEN to the Teti demethylase catalytic domain (see e.g., Maeder et al. Nature Biotechnology 31(12):1137-42 (2013)) or a TAL effector fused to LSD1 histone demethylase (Mendenhall et al. Nature Biotechnology 31(12):1133-6 (2013)).

(i) Modified DNA Endonucleases, Nuclease-Dead Cas9 and Uses Thereof

Unlike viral vectors, the ceDNA vectors as described herein do not have a capsid that limits the size or number of nucleic acid sequences, effector sequences, regulatory sequences etc. that can be delivered to a cell. Accordingly, ceDNA vectors as described herein can comprise nucleic acids encoding nuclease-dead DNA endonucleases, nickases, or other DNA endonucleases with modified function (e.g., unique PAM binding sequence) for enhanced production of a desired vector and/or delivery of the vector to a cell. Such ceDNA vectors can also include promoter sequences and other regulatory or effector sequences as desired. Given the lack of size constraint, one of skill in the art will readily understand that, for example, that expression of a desired nuclease with modified function, and optionally, at least one guide RNA can be from nucleic acid sequences on the same vector and can be under the control of the same or different promoters. It is also contemplated herein that at least two different modified endonucleases can be encoded in the same vector, for example, for multiplexed gene expression modulation (see “Multiplexed gene expression modulation” section herein) and under the control of the same or different promoters. Thus, one of skill in the art could combine the desired functionality of at least two different Cas9 endonucleases (e.g., at least 3, at least 4, at least 5, at least 6, at least 7, at least 8, at least 9, at least 10, or more) as desired including, for example, temporally regulated expression of at least two different modified endonucleases by one or more inducible promoters.

In some embodiments, a DNA endonuclease for use with the methods and compositions described herein, can be modified such that the DNA endonuclease retains DNA binding activity e.g., at a target site of the genome determined by a guide RNA sequence but does not retain cleavage activity (e.g., nuclease dead Cas9 (dCas9)) or has reduced cleavage activity (e.g., by at least 10%, at least 20%, at least 30%, at least 40%, at least 50%, at least 60%, at least 70%, at least 80%, at least 90%, at least 95%, at least 99%) as compared to the unmodified DNA endonuclease (e.g., Cas9 nickase). In some embodiments, a modified DNA endonuclease is used herein to inhibit expression of a target gene. For example, since a modified DNA endonuclease retains DNA binding activity, it can prevent the binding of RNA polymerase and/or displace RNA polymerase, which in turn prevents transcription of the target gene. Thus, expression of a gene product (e.g., mRNA, protein) from the desired gene is prevented.

For example, a “deactivated Cas9 (dCas9),” “nuclease dead Cas9” or an otherwise inactivated form of Cas9 can be introduced with a guide RNA that directs binding to a specific gene. Such binding can reduce in inhibition of expression of the target gene, if desired. In some embodiments, one may want to have the ability to reverse such gene expression inhibition. This can be achieved, for example, by providing different guide RNAs to the dead Cas9 protein to weaken the binding of Cas9 to the genomic site. Such reversal can occur in an iterative fashion where at least two or a series of guide RNAs designed to decrease the stability of the dead Cas9 binding are administered in succession. For example, each successive guide RNA can increase the instability from the degree of instability/stability of dead Cas9 binding produced by the guide RNA in the previous iteration. Thus, in some embodiments, one can use a dCas9 directed to a target gene sequence with a guide RNA to “inactivate a desired gene,” without cleavage of the genomic sequence, such that the gene of interest is not expressed in a functional protein form. In alternative embodiments, a guide RNA can be designed such that the stability of the dCas9 binding is reduced, but not eliminated. That is, the displacement of RNA polymerase is not complete thereby permitting the “reduction of gene expression” of the desired gene.

In certain embodiments, hybrid recombinases may be suitable for use in ceDNA vectors of the present disclosure to create integration cites on target DNA. For example, Hybrid recombinases based on activated catalytic domains derived from the resolvase/invertase family of serine recombinases fused to Cyst-Hist zinc-finger or TAL effector DNA-binding domains are a class of reagents capable improved targeting specificity in mammalian cells and achieve excellent rates of site-specific integration. Suitable hybrid recombinases encoded by nucleotides in ceDNA vectors in accordance with the present disclosure include those described in Gaj et al., Enhancing the Specificity of Recombinase-Mediated Genome Engineering through Dimer Interface Redesign, Journal of the American Chemical Society, Mar. 10, 2014 (herein incorporated by reference in its entirety).

(ii) Zinc Finger Endonucleases and TALENs

ZFNs and TALEN-based restriction endonuclease technology utilizes a non-specific DNA cutting enzyme which is linked to a specific DNA sequence recognizing peptide(s) such as zinc fingers and transcription activator-like effectors (TALEs). Typically, an endonuclease whose DNA recognition site and cleaving site are separate from each other is selected and its cleaving portion is separated and then linked to a sequence recognizing peptide, thereby yielding an endonuclease with very high specificity for a desired sequence. An exemplary restriction enzyme with such properties is FokI. Additionally, FokI has the advantage of requiring dimerization to have nuclease activity and this means the specificity increases dramatically as each nuclease partner recognizes a unique DNA sequence. To enhance this effect, FokI nucleases have been engineered that can only function as heterodimers and have increased catalytic activity. The heterodimer functioning nucleases avoid the possibility of unwanted homodimer activity and thus increase specificity of the double-stranded break.

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combination in their proteins. Cys2-His2 Zinc fingers typically happen in repeats that are 3 bp apart and are found in diverse combinations in a variety of nucleic acid interacting proteins such as transcription factors. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs for use with the methods and compositions described herein can be obtained commercially from e.g., Sangamo Biosciences™ (Richmond, Calif.).

The terms “Transcription activator-like effector nucleases” or “TALENs” as used interchangeably herein refers to engineered fusion proteins of the catalytic domain of a nuclease, such as endonuclease FokI, and a designed TALE DNA-binding domain that may be targeted to a custom DNA sequence. A “TALEN monomer” refers to an engineered fusion protein with a catalytic nuclease domain and a designed TALE DNA-binding domain. Two TALEN monomers may be designed to target and cleave a TALEN target region.

The terms “Transcription activator-like effector” or “TALE” as used herein refers to a protein structure that recognizes and binds to a particular DNA sequence. The “TALE DNA-binding domain” refers to a DNA-binding domain that includes an array of tandem 33-35 amino acid repeats, also known as RVD modules, each of which specifically recognizes a single base pair of DNA. RVD modules can be arranged in any order to assemble an array that recognizes a defined sequence. A binding specificity of a TALE DNA-binding domain is determined by the RVD array followed by a single truncated repeat of 20 amino acids. A TALE DNA-binding domain may have 12 to 27 RVD modules, each of which contains an RVD and recognizes a single base pair of DNA. Specific RVDs have been identified that recognize each of the four possible DNA nucleotides (A, T, C, and G). Because the TALE DNA-binding domains are modular, repeats that recognize the four different DNA nucleotides may be linked together to recognize any particular DNA sequence. These targeted DNA-binding domains can then be combined with catalytic domains to create functional enzymes, including artificial transcription factors, methyltransferases, integrases, nucleases, and recombinases.

The TALENs may include a nuclease and a TALE DNA-binding domain that binds to the target sequence or gene in a TALEN target region. A “TALEN target region” includes the binding regions for two TALENs and the spacer region, which occurs between the binding regions. The two TALENs bind to different binding regions within the TALEN target region, after which the TALEN target region is cleaved. Examples of TALENs are described in International Patent Application WO2013103628, which is incorporated by reference in its entirety.

The terms “Zinc finger nuclease” or “ZFN” as used interchangeably herein refers to a chimeric protein molecule comprising at least one zinc finger DNA binding domain effectively linked to at least one nuclease or part of a nuclease capable of cleaving DNA when fully assembled. “Zinc finger” as used herein refers to a protein structure that recognizes and binds to DNA sequences. The zinc finger domain is the most common DNA-binding motif in the human proteome. A single zinc finger contains approximately 30 amino acids and the domain typically functions by binding 3 consecutive base pairs of DNA via interactions of a single amino acid side chain per base pair.

In certain embodiments, ceDNA vectors in accordance with the present disclosure include nucleotide sequences encoding zinc-finger recombinases (ZFR) or chimeric proteins suitable for introducing targeted modifications into cells, such as mammalian cells. Unlike targeted nucleases and conventional SSR systems, ZFR specificity is the cooperative product of modular site-specific DNA recognition and sequence-dependent catalysis. ZFR's with diverse targeting capabilities can be generated with a plug-and-play manner. ZFR's including enhanced catalytic domains demonstrate improved targeting specificity and efficiency, and enable the site-specific delivery of therapeutic genes into the human genome with low toxicity. Mutagenesis of the Cre recombinase dimer interface also improves recombination specificity.

In embodiments, ceDNA vectors in accordance with the present disclosure are suitable for use in nuclease free HDR systems such as those described in Porro et al., Promoterless gene targeting without nucleases rescues lethality of a Crigler-Najjar syndrome mouse model, EMBO Molecular Medicine, Jul. 27, 2017 (herein incorporated by reference in its entirety). In such embodiments, in vivo gene targeting approaches are suitable for ceDNA application based on the insertion of a donor sequence, without the use of nucleases. In some embodiments, the donor sequence may be promoterless.

While TALEN and ZFN are exemplified for use of the ceDNA vector for DNA editing (e.g., genomic DNA editing), also encompassed herein are use of mtZFN and mitoTALEN function, or mitochondrial-adapted CRISPR/Cas9 platform for use of the ceDNA vectors for editing of mitochondrial DNA (mtDNA), as described in Maeder, et al. “Genome-editing technologies for gene and cell therapy.” Molecular Therapy 24.3 (2016): 430-446 and Gammage P A, et al. Mitochondrial Genome Engineering: The Revolution May Not Be CRISPR-Ized. Trends Genet. 2018; 34(2):101-110.

(iii) Nucleic Acid-Guided Endonucleases

Different types of nucleic acid-guided endonucleases can be used in the compositions and methods of the invention to facilitate ceDNA-mediated gene editing. Exemplary, nonlimiting, types of nucleic acid-guided endonucleases suited for the compositions and methods of the invention include RNA-guided endonucleases, DNA-guided endonucleases, and single-base editors.

In some embodiments, the nuclease can be an RNA-guided endonuclease. As used herein, the term “RNA-guided endonuclease” refers to an endonuclease that forms a complex with an RNA molecule that comprises a region complementary to a selected target DNA sequence, such that the RNA molecule binds to the selected sequence to direct endonuclease activity to the selected target DNA sequence.

In one embodiment, the RNA-guided endonuclease is a CRISPR enzyme, as discussed herein. In some embodiments, the RNA-guided endonuclease comprises nickase activity. In some embodiments, the RNA-guided endonuclease directs cleavage of one or both strands at the location of a target sequence, such as within the target sequence and/or within the complement of the target sequence. In some embodiments, the RNA-guided endonuclease directs cleavage of one or both strands within about 1, 2, 3, 4, 5, 6, 7, 8, 9, 10, 15, 20, 25, 50, 100, 200, 500, or more base pairs from the first or last nucleotide of a target sequence. In other embodiments, the nickase activity is directed to one or more sequences on the ceDNA vectors themselves, for example, to loosen the sequence constraint such that the HDR template is exposed for HDR interaction with the genomic sequence of the target gene.

In certain embodiments, it is contemplated that the nickase cuts at least 1 site, at least 2 sites, at least 3 sites, at least 4 sites, at least 5 sites, at least 6 sites, at least 7 sites, at least 8 sites, at least 9 sites, at least 10 sites or more on the desired nucleic acid sequence (e.g., one or more regions of the ceDNA vector). In another embodiment, it is contemplated that the nickase cuts at 1 and/or 2 sites via trans-nicking. Trans-nicking can enhance genomic editing by HDR, which is high-fidelity, introduces fewer errors, and thus reduces unwanted off-target effects.

In some embodiments, an expression construct or vector encodes an RNA-guided endonuclease that is mutated with respect to a corresponding wild-type enzyme such that the mutated endonuclease lacks the ability to cleave one strand of a target polynucleotide containing a target sequence.

In some embodiments, the nucleic acid sequence encoding the RNA-guided endonuclease is codon optimized for expression in particular cells, such as eukaryotic cells. The eukaryotic cells can be derived from a particular organism, such as a mammal. Non-limiting examples of mammals can include human, mouse, rat, rabbit, dog, or non-human primate. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence.

In some embodiments, the RNA-guided endonuclease is part of a fusion protein comprising one or more heterologous protein domains (e.g., about or more than about 1, 2, 3, 4, 5, 6, 7, 8, 9, 10, or more domains in addition to the endonuclease). An RNA-guided endonuclease fusion protein can comprise any additional protein sequence, and optionally a linker sequence between any two domains. Examples of protein domains that can be fused to an RNA-guided endonuclease include, without limitation, epitope tags, reporter gene sequences, purification tags, fluorescent proteins and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, glutathione-S-transferase (GST), chitin binding protein (CBP), maltose binding protein (MBP), poly(NANP), tandem affinity purification (TAP) tag, myc, AcV5, AU1, AUS, E, ECS, E2, nus, Softag 1, Softag 3, Strep, SBP, Glu-Glu, HSV, KT3, S, SI, T7, biotin carboxyl carrier protein (BCCP), calmodulin, and thioredoxin (Trx) tags. Examples of reporter genes include, but are not limited to, glutathione-S-transferase (GST), horseradish peroxidase (HRP), chloramphenicol acetyltransferase (CAT) beta-galactosidase, beta-glucuronidase, luciferase, green fluorescent proteins (e.g., GFP, GFP-2, tagGFP, turboGFP, sfGFP, EGFP, Emerald, Azami Green, Monomeric Azami Green, CopGFP, AceGFP, ZsGreen1), HcRed, DsRed, cyan fluorescent protein (CFP), yellow fluorescent proteins (e.g., YFP, EYFP, Citrine, Venus YPet, PhiYFP, ZsYellow1), cyan fluorescent proteins (e.g., ECFP, Cerulean, CyPet AmCyanl, Midoriishi-Cyan) red fluorescent proteins (e.g., mKate, mKate2, mPlum, DsRed monomer, mCherry, mRFP1, DsRed-Express, DsRed2, HcRed-Tandem, HcRed1, AsRed2, eqFP611, mRaspberry, mStrawberry, Jred), orange fluorescent proteins (e.g., mOrange, mkO, Kusabira-Orange, monomeric Kusabira-Orange, mTangerine, tdTomato) and autofluorescent proteins including blue fluorescent protein (BFP). An RNA-guided endonuclease can be fused to a gene sequence encoding a protein or a fragment of a protein that binds DNA molecules or binds to other cellular molecules, including but not limited to maltose binding protein (MBP), S-tag, Lex A DNA binding domain (DBD) fusions, GAL4 DNA binding domain fusions, and herpes simplex virus (HSV) BP16 protein fusions. In some embodiments, a tagged endonuclease is used to identify the location of a target sequence.

It is contemplated herein that at least two (e.g., at least 3, at least 4, at least 5, at least 6, at least 7, at least 8, at least 9, at least 10, at least 12, at least 15 or more) different Cas enzymes are administered or are in contact with a cell at substantially the same time. Any combination of double-stranded break-inducing Cas enzymes, Cas nickases, catalytically inactive Cas enzymes (e.g., dCas9), modified Cas enzymes, truncated Cas9, etc. are contemplated for use in combination with the methods and compositions described herein.

In some embodiments, the nucleic acid-guided endonuclease is a DNA-guided endonuclease. See, e.g., Varshney and Burgess Genome Biol. 17:187 (2016). In one embodiment, an enzyme involved in DNA repair and/or replication may be fused to an endonuclease to form a DNA-guided nuclease. One nonlimiting example is the fusion of flap endonuclease 1 (FEN-1) to the FokI endonuclease (Xu et al., Genome Biol. 17:186 (2016). In another embodiment, naturally-occurring DNA-guided nucleases may be used. Nonlimiting examples of such naturally-occurring nucleases are prokaryotic endonucleases from the Argonaute protein family (Kropocheva et al., FEBS Open Bio. 8(S1): P01-074 (2018). In some embodiments, the nucleic acid-guided endonuclease is a “single-base editor”, which is a chimeric protein composed of a DNA targeting module and a catalytic domain capable of modifying a single type of nucleotide base (Rusk, N, Nature Methods 15:763 (2018); Eid et al., Biochem J. 475(11): 1955-64 (2018)). Because such single-base editors do not generate double-strand breaks in the target DNA to effect the editing of the DNA base, the generation of insertions and deletions (e.g., indels) is limited, thus improving the fidelity of the editing process. Different types of single base editors are known. For example, cytidine deaminases (enzymes that catalyze the conversion of cytosine into uracil) may be coupled to nucleases such as APOBEC-dCas9—where APOBEC contributes the cytidine deaminase functionality and is guided by dCas9 to deaminate a specific cytidine to uracil. The resulting U-G mismatches are resolved via repair mechanisms and form U-A base pairs, which translate into C-to-T point mutations (Komor et al., Nature 533: 420-424 (2016); Shimatani et al., Nat. Biotechnol. 35: 441-443 (2017)). Adenine deaminase-based DNA single base editors have been engineered. They deaminate adenosine to form inosine, which can base pair with cytidine and be corrected to guanine such that an A-T pair may be converted to a G-C pair. Examples of such editors include TadA, ABE5.3, ABE7.8, ABE7.9, and ABE7.10 (Gaudelli et al., Nature 551: 464-471 (2017).

(iv) CRISPR/Cas Systems

As known in the art, a CRISPR-CAS9 system is a particular set of nucleic-acid guided-nuclease-based systems that includes a combination of protein and ribonucleic acid (“RNA”) that can alter the genetic sequence of an organism. The CRISPR-CAS9 system continues to develop as a powerful tool to modify specific deoxyribonucleic acid (“DNA”) in the genomes of many organisms such as microbes, fungi, plants, and animals. For example, mouse models of human disease can be developed quickly to study individual genes much faster, and easily change multiple genes in cells at once to study their interactions. One of ordinary skill in the art may select between a number of known CRISPR systems such as Type I, Type II, and Type III. Type II CRISPR-CAS system has a well-known mechanism including three components: (1) a crDNA molecule, which is called a “guide sequence” or “targeter-RNA”; (2) a “tracr RNA” or “activator-RNA”; and (3) a protein called Cas9.

To alter the DNA molecule, a number of interactions occur in the system including: (1) the guide sequence binding by specific base pairing to a specific sequence of DNA of interest (“target DNA”), (2) the guide sequence binds by specific base pairing at another sequence to an activator-RNA, and (3) activator-RNA interacts with the Cas protein (e.g., Cas9 protein), which then acts as a nuclease to cut the target DNA at a specific site. Suitable systems for use in accordance with ceDNA vectors in accordance with the present disclosure are further described in Van Nierop, et al. Stimulation of homology-directed gene targeting at an endogenous human locus by a nicking endonuclease, Nucleic Acid Research, August 2009 and Ran et al., Double nicking by RNA-guided CRISPR Cas9 for enhanced genome editing specificity.

ceDNA vectors in accordance with the present disclosure can be designed to include nucleotides encoding one or more components of these systems such as the guide sequence, tracr RNA, or Cas (e.g., Cas9). In certain embodiments, a single promoter drives expression of a guide sequence and tracr RNA, and a separate promoter drives Cas (e.g., Cas9) expression. One of skill in the art will appreciate that certain Cas nucleases require the presence of a protospacer adjacent motif (PAM) adjacent to a target nucleic acid sequence. In some embodiments, the PAM may be adjacent to or within 1, 2, 3, or 4 nucleotides of the 3′ end of the target sequence. The length and the sequence of the PAM can depend on the particular Cas protein. Exemplary PAM sequences include NGG, NGGNG, NG, NAAAAN, NNAAAAAW, NNNNACA, GNNNCNNA, TTN and NNNNGATT (wherein N is defined as any nucleotide and W is defined as either A or T). In some embodiments, the PAM sequence can be on the guide RNA, for example, when editing RNA.

In some embodiments, RNA-guided nucleases including Cas and Cas9 are suitable for use in ceDNA vectors designed to provide one or more components for genome engineering using the CRISPR-Cas9 system See e.g. US publication 2014/0170753 herein incorporated by reference in its entirety. CRISPR-Cas 9 provides a set of tools for Cas9-mediated genome editing via non-homologous end joining (NHEJ) or homology-directed repair (HDR) in mammalian cells, as well as generation of modified cell lines for downstream functional studies. To minimize off-target cleavage, the CRISPR-Cas9 system may include a double-nicking strategy using the Cas9 nickase mutant with paired guide RNAs. This system is known in the art, and described in, for example, Ran et al., Genome engineering using the CRISPR-Cas9 system, Nature Protocols, 24 Oct. 2013, and Zhang, et al., Efficient precise knockin with a double cut HDR donor after CRISPR/Cas9-mediated double-stranded DNA cleavage, Genome Biology, 2017 (both references are herein incorporated by reference in their entirety).

In certain embodiments, the ceDNA system includes a nuclease and guide RNAs that are directed to a ceDNA sequence. For example, a nicking CAS, such as nCAS9 D10A can be used to increase the efficiency of gene editing. The guide RNAs can direct nCAS nicking of the ceDNA thereby releasing torsional constraints of ceDNA for more efficient gene repair and/or expression. Using a nicking nuclease relieves the torsional constraints while retaining sequence and structural integrity allowing the nicked DNA can persist in the nucleus. The guide RNAs can be directed to the same strand of DNA or the complementary strand. The guide RNAs can be directed to e.g., the ITRS, or sequences proceeding promoters, or homology domains etc.

In one embodiment, the RNA-guided endonuclease is a CRISPR enzyme, such as a Cas protein. Non-limiting examples of Cas proteins include Cas1, Cas1B, Cas2, Cas3, Cas4, Cas5, Cas5e (CasD), Cas6, Cas6e, Cas6f, Cas7, Cas8, Cas8a1, Cas8a2, Cas8b, Cas8c, Cas9 (also known as Csn1 and Csx12), Cas10, Cas10d, Cas13, Cas13a, Cas13c, CasF, CasH, Csy1, Csy2, Csy3, Cse1, Cse2, Cse3, Cse4, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx11, Csx16, CsaX, Cszl, Csx3, Csx1, Csx15, Csf1, Csf2, Csf3, Csf4, Cul966, Cpf1, C2c1, C2c3, homologs thereof, or modified versions thereof. In one embodiment, the Cas protein is Cas9. In another embodiment, the Cas protein is nuclease-dead Cas9 (dCas9) or a Cas9 nickase. In one embodiment, the Cas protein is a nicking Cas enzyme (nCas).

Typically, the RNA-guided endonuclease comprises DNA cleavage activity, such as the double strand breaks initiated by Cas9. In some embodiments, the RNA-guided endonuclease is Cas9, for example, Cas9 from S. pyogenes or S. pneumoniae. Other non-limiting bacterial sources of Cas9 include Streptococcus pyogenes, Streptococcus pasteurianus Streptococcus thermophilus, Streptococcus sp., Nocardiopsis dassonvillei, Streptomyces pristinaespiralis, Streptomyces viridochromogenes, Streptosporangium roseum, Streptosporangium roseum, Staphylococcus aureus, Alicyclobaccillus acidocaldarius, Bacillus pseudomycoides, Bacillus selenitireducens, Exiguobacterium sibiricum, Francisella novic ida, Wolinella succinogenes, Lactobacillus delbrueckii, Lactobacillus salivarius, Listeria innocua, Lactobacillus gasseri, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Fibrobacter succinogene, Natranaerobius thermophilus, Pelotomaculumthermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Sutterella wadsworthensis, Gamma proteobacterium, Neisseria cinerea, Neisseria meningitidis, Campylobacter jejuni, Campylobacter lari, Parvibaculum lavamentivorans, Comeybacterium diphtheria, Pasteurella multocida, Rhodospirillum rubrum, Nocardiopsis dassonvillei, or Acaryochloris marina.

In one embodiment, the Cas9 nickase comprises nCas9 D10A. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A. In some embodiments, a Cas9 nickase can be used in combination with guide sequence(s), e.g., two guide sequences, which target respectively sense and antisense strands of the DNA target. This combination allows both strands to be nicked and used to induce non-homologous end joining (NHEJ) repair.

In some embodiments, the RNA-guided endonuclease is Cas13. A catalytically inactive Cas13 (dCas13) can be used to edit mRNA sequences as described in e.g., Cox, D et al. RNA editing with CRISPR-Cas13 Science (2017) DOI: 10.1126/science.aaq0180, which is herein incorporated by reference in its entirety.

In some embodiments, the ceDNA vector as described herein encoding an endonuclease is Cas9 (e.g., SEQ ID NO: 829), or an amino acid or functional fragment of a nuclease having at least 60%, more preferably at least 65%, more preferably at least 70%, more preferably at least 75%, more preferably at least 80%, more preferably at least 85%, even more preferably at least 90%, and most preferably at least 95% sequence identity to SEQ ID NO:829 (Cas9) or consisting of SEQ ID NO: 829. In certain embodiments, Cas 9 includes one or more mutations in a catalytic domain rendering the Cas 9 a nickase that cleaves a single DNA strand, such as those described in U.S. Patent Publication No. 2017-0191078-A9 (incorporated by reference in its entirety).

In some embodiments, the ceDNA vectors of the present disclosure are suitable for use in systems and methods based on RNA-programmed Cas9 having gene-targeting and genome editing functionality. For example, the ceDNA vectors of the present disclosure are suitable for use with Clustered Regularly Interspaced Short Palindromic Repeats or the CRISPR associated (Cas) systems for gene targeting and gene editing. CRISPR cas9 systems are known in the art and described, e.g., in U.S. patent application Ser. No. 13/842,859 filed on March 2013, and U.S. Pat. Nos. 8,697,359, 8,771,945, 8,795,965, 8,865,406, 8,871,445 all of which are herein incorporated by reference in their entirety.

It is also contemplated herein that Cas9, a Cas9 nickase, or a deactivated Cas9 (dCas9, or also referred to a nuclease dead Cas9 or “catalytically inactive”) are also prepared as fusion proteins with FokI, such that gene editing or gene expression modulation occurs upon formation of Fold heterodimers.

Further, dCas9 can be used to activate (CRISPRa) or inhibit (CRISPRi) expression of a desired gene at the level of regulatory sequences upstream of the target gene sequence. CRISPRa and CRISPRi can be performed, for example, by fusing dCas9 with an effector region (e.g., dCas9/effector fusion) and supplying a guide RNA that directs the dCas9/effector fusion protein to bind to a sequence upstream of the desired or target gene (e.g., in the promoter region). Since dCas9 has no nuclease activity, it remains bound to the target site in the promoter region and the effector portion of the dCas9/effector fusion protein can recruit transcriptional activators or repressors to the promoter site. As such, one can activate or reduce gene expression of a target gene as desired. Previous work in the literature indicates that the use of a plurality of guide RNAs co-expressed with dCas9 can increase expression of a desired gene (see e.g., Maeder et al. CRISPR RNA-guided activation of endogenous human genes Nat Methods 10(10):977-979 (2013). In some embodiments, it is desirable to permit inducible repression of a desired gene. This can be achieved, for example, by using guide RNA binding sites in promoter regions upstream of the transcription start site (see e.g., Gao et al. Complex transcriptional modulation with orthogonal and inducible dCas9 regulators. Nature Methods (2016)). In some embodiments, a nuclease dead version of a DNA endonuclease (e.g., dCas9) can be used to inducibly activate or increase expression of a desired gene, for example, by introduction of an agent that interacts with an effector domain (e.g., a small molecule or at least one guide RNA) of a dCas9/effector fusion protein. In other embodiments, it is also contemplated herein that dCas9 can be fused to a chemical- or light-inducible domain, such that gene expression can be modulated using extrinsic signals. In one embodiment, inhibition of a target gene's expression is performed using dCas9 fused to a KRAB repressor domain, which may be beneficial for improved inhibition of gene expression in mammalian systems and have few off-target effects. Alternatively, transcription-based activation of a gene can be performed using a dCas9 fused to the omega subunit of RNA polymerase, or the transcriptional activators VP64 or p65.

Accordingly, in some embodiments, the methods and compositions described herein, e.g., ceDNA vectors can comprise and/or be used to deliver CRISPRi (CRISPR interference) and/or CRISPRa (CRISPR activation) systems to a host cell. CRISPRi and CRISPRa systems comprise a deactivated RNA-guided endonuclease (e.g., Cas9) that cannot generate a double strand break (DSB). This permits the endonuclease, in combination with the guide RNAs, to bind specifically to a target sequence in the genome and provide RNA-directed reversible transcriptional control. In one embodiment, the ceDNA vector comprises a nucleic acid encoding a nuclease and/or a guide RNA but does not comprise a homology directed repair template or corresponding homology arms.

In some embodiments of CRISPRi, the endonuclease can comprise a KRAB effector domain. Either with or without the KRAB effector domain, the binding of the deactivated nuclease to the genomic sequence can, e.g., block transcription initiation or progression and/or interfere with the binding of transcriptional machinery or transcription factors.

In CRISPRa, the deactivated endonuclease can be fused with one or more transcriptional activation domains, thereby increasing transcription at or near the site targeted by the endonuclease. In some embodiments, CRISPRa can further comprise gRNAs which recruit further transcriptional activation domains. sgRNA design for CRISPRi and CRISPRa is known in the art (see, e.g., Horlbeck et al. eLife. 5, e19760 (2016); Gilbert et al., Cell. 159, 647-661 (2014); and Zalatan et al., Cell. 160, 339-350 (2015); each of which is incorporated by reference here in its entirety). CRISPRi and CRISPRa-compatible sgRNA can also be obtained commercially for a given target (see, e.g., Dharmacon; Lafayette, Colo.). Further description of CRISPRi and CRISPRa can be found, e.g., in Qi et al., Cell. 152, 1173-1183 (2013); Gilbert et al., Cell. 154, 442-451 (2013); Cheng et al., Cell Res. 23, 1163-1171 (2013); Tanenbaum et al. Cell. 159, 635-646 (2014); Konermann et al., Nature. 517, 583-588 (2015); Chavez et al., Nat. Methods. 12, 326-328 (2015); Liu et al., Science. 355 (2017); and Goyal et al., Nucleic Acids Res. (2016); each of which is incorporated by reference herein in its entirety.

Accordingly, in some embodiments described herein is a ceDNA vector comprising a deactivated endonuclease, e.g., RNA-guided endonuclease and/or Cas9, wherein the deactivated endonuclease lacks endonuclease activity, but retains the ability to bind DNA in a site-specific manner, e.g., in combination with one or more guide RNAs and/or sgRNAs. In some embodiments, the vector can further comprise one or more tracrRNAs, guide RNAs, or sgRNAs. In some embodiments, the deactivated endonuclease can further comprise a transcriptional activation domain. In some embodiments, ceDNA vectors of the present disclosure are also useful for deactivated nuclease systems, such as CRISPRi or CRISPRa dCas systems, nCas, or Cas13 systems, all well known in the art.

It is also contemplated herein that the vectors described herein can be used in combination with dCas9 to visualize genomic loci in living cells (see e.g., Ma et al. Multicolor CRISPR labeling of chromosomal loci in human cells PNAS 112(10):3002-3007 (2015)). CRISPR mediated visualization of the genome and its organization within the nucleus is also called the 4-D nucleome. In one embodiment, dCas9 is modified to comprise a fluorescent tag. Multiple loci can be labeled in distinct colors, for example, using orthologs that are each fused to a different fluorescent label. This technique can be expanded to study genome structure, for example, by using guide RNAs that bind Alu sequences to aid in mapping the location of guide RNA-specified repeats (see e.g., McCaffrey et al. Nucleic Acids Res 44(2):e11 (2016)). Thus, in some embodiments, mapping of clinically significant loci is contemplated herein, for example, for the identification and/or diagnosis of Huntington's disease, among others. Methods of performing genome visualization or genetic screens with a ceDNA vector(s) encoding a gene editing system are known in the art and/or are described in, for example, Chen et al. Cell 155:1479-1491 (2013); Singh et al. Nat Commun 7:1-8 (2016); Korkmaz et al. Nat Biotechnol 34:1-10 (2016); Hart et al. Cell 163:1515-1526 (2015); the contents of each of which are incorporated herein by reference in their entirety.

In some embodiments, it may be desirable to edit a single base in the genome, for example, modifying a single nucleotide polymorphism associated with a particular disease (see e.g., Komor, A C et al. Nature 533:420-424 (2016); Nishida, K et al. Targeted nucleotide editing using hybrid prokaryotic and vertebrate adaptive immune systems. Science (2016)). Single nucleotide base editing makes use of base-converting enzyme tethered to a catalytically inactive endonuclease (e.g., nuclease dead Cas9) that does not cut the target gene loci. After the base conversion by a base editing enzyme, the system makes a nick on the opposite, unedited strand, which is repaired by the cell's own DNA repair mechanisms. This results in the replacement of the original nucleotide, which is now a “mismatched nucleotide,” thus completing the conversion of a single nucleotide base pair. Endogenous enzymes are available for effecting the conversion of G/C nucleotide pairs to A/T nucleotide pairs, for example, cytidine deaminase, however there is no endogenous enzyme for catalyzing the reverse conversion of A/T nucleotide pairs to G/C ones. Adenine deaminases (e.g., TadA), that usually only act on RNA to convert adenine to inosine, have been evolutionarily selected for in bacterial systems to identify adenine deaminase mutants that act on DNA to convert adenosine to inosine (see e.g., Gaudelli et al Nature (2017), in press doi:10.1038/nature24644, the contents of which are incorporated by reference in its entirety).

In some embodiments, dCas9 or a modified Cas9 with a nickase function can be fused to an enzyme having a base editing function (e.g., cytidine deaminase APOBEC1 or a mutant TadA). The base editing efficiency can be further improved by including an inhibitor of endogenous base excision repair systems that remove uracil from the genomic DNA. See Gaudelli et al. (2017) programmable base editing of A-T to G-C in genomic DNA without DNA cleavage, Nature Published online 25 Oct. 2017, herein incorporated by reference in its entirety.

It is also contemplated herein that the desired endonuclease is modified by addition of ubiquitin or a polyubiquitin chain. In some embodiments, the ubiquitin can be a ubiquitin-like protein (UBL). Non-limiting examples of ubiquitin-like proteins include small ubiquitin-like modifier (SUMO), ubiquitin cross-reactive protein (UCRP, also known as interferon-stimulated gene 15 (ISG-15)), ubiquitin-related modifier-1 (URM1), neuronal-precursor-cell-expressed developmentally downregulated protein-8 (NEDD8, also called Rubl in S. cerevisiae), human leukocyte antigen F-associated (FAT 10), autophagy-8 (ATG8) and -12 (ATG12), Fau ubiquitin-like protein (FUB1), membrane-anchored UBL (MUB), ubiquitin fold-modifier-1 (UFM1), and ubiquitin-like protein-5 (UBL5).

CeDNA vectors or compositions thereof can encode for modified DNA endonucleases as described in e.g., Fu et al. Nat Biotechnol 32:279-284 (2013); Ran et al. Cell 154:1380-1389 (2013); Mali et al. Nat Biotechnol 31:833-838 (2013); Guilinger et al. Nat Biotechnol 32:577-582 (2014); Slaymaker et al. Science 351:84-88 (2015); Klenstiver et al. Nature 523:481-485 (2015); Bolukbasi et al. Nat Methods 12:1-9 (2015); Gilbert et al. Cell 154; 442-451 (2012); Anders et al. Mol Cell 61:895-902 (2016); Wright et al. Proc Natl Acad Sci USA 112:2984-2989 (2015); Truong et al. Nucleic Acids Res 43:6450-6458 (2015); the contents of each of which are incorporated herein by reference in their entirety.

(v) MegaTALS

In some embodiments, the endonuclease described herein can be a megaTAL. MegaTALs are engineered fusion proteins which comprise a transcription activator-like (TAL) effector domain and a meganuclease domain. MegaTALs retain the ease of target specificity engineering of TALs while reducing off-target effects and overall enzyme size and increasing activity. MegaTAL construction and use is described in more detail in, e.g., Boissel et al. 2014 Nucleic Acids Research 42(4):2591-601 and Boissel 2015 Methods Mol Biol 1239:171-196; each of which is incorporated by reference herein in its entirety. Protocols for megaTAL-mediated gene knockout and gene editing are known in the art, see, e.g., Sather et al. Science Translational Medicine 2015 7(307):ra156 and Boissel et al. 2014 Nucleic Acids Research 42(4):2591-601; each of which is incorporated by reference herein in its entirety. MegaTALs can be used as an alternative endonuclease in any of the methods and compositions described herein.

(vi) Multiplex Modulation of Gene Expression and Complex Systems

The lack of size limitations of the ceDNA vectors as described herein are especially useful in multiplexed editing, CRISPRa or CRISPRi because multiple guide RNAs can be expressed from the same ceDNA vector, if desired. CRISPR is a robust system and the addition of multiple guide RNAs does not substantially alter the efficiency of gene editing, CRISPRa, CRISPRi or CRISPR mediated labeling of nucleic acids. As described elsewhere, the plurality of guide RNAs can be under the control of a single promoter (e.g., a polycistronic transcript) or under the control of a plurality of promoters (e.g., at least 2, at least 3, at least 4, at least 5, at least 6, etc. up to a limit of a 1:1 ratio of guide RNA:promoter sequences).

The multiplex CRISPR/Cas9-Based System takes advantage of the simplicity and low cost of sgRNA design and may be helpful in exploiting advances in high-throughput genomic research using CRISPR/Cas9 technology. For example, the ceDNA vectors described herein are useful in expressing Cas9 and numerous single guide RNAs (sgRNAs) in difficult cell lines. The multiplex CRISPR/Cas9-Based System may be used in the same ways as the CRISPR/Cas9-Based System described above. Multiplex CRISPR/Cas can be performed as described in Cong, L et al. Science 819 (2013); Wang et al. Cell 153:910-918 (2013); Ma et al. Nat Biotechnol 34:528-530 (2016); the contents of each of which are incorporated herein by reference in their entirety.

In addition to the described transcriptional activation and nuclease functionality, this system will be useful for expressing other novel Cas9-based effectors that control epigenetic modifications for diverse purposes, including interrogation of genome architecture and pathways of endogenous gene regulation. As endogenous gene regulation is a delicate balance between multiple enzymes, multiplexing Cas9 systems with different functionalities will allow for examining the complex interplay among different regulatory signals. The vector described here should be compatible with aptamer-modified gRNAs and orthogonal Cas9s to enable independent genetic manipulations using a single set of gRNAs.

The multiplex CRISPR/Cas9-Based System may be used to activate at least one endogenous gene in a cell. The method includes contacting a cell with the modified lentiviral vector. The endogenous gene may be transiently activated or stably activated. The endogenous gene may be transiently repressed or stably repressed. The fusion protein may be expressed at similar levels to the sgRNAs. The fusion protein may be expressed at different levels compared to the sgRNAs. The cell may be a primary human cell.

The multiplex CRISPR/Cas9-Based System may be used in a method of multiplex gene editing in a cell. The method includes contacting a cell with a ceDNA vector. The multiplex gene editing may include correcting a mutant gene or inserting a transgene. Correcting a mutant gene may include deleting, rearranging or replacing the mutant gene. Correcting the mutant gene may include nuclease-mediated non-homologous end joining or homology-directed repair. The multiplex gene editing may include deleting or correcting at least one gene, wherein the gene is an endogenous normal gene or a mutant gene.

The multiplex gene editing may include deleting or correcting at least two genes. For example, at least two genes, at least three genes, at least four genes, at least five genes, at least six genes, at least seven genes, at least eight genes, at least nine genes, or at least ten genes may be deleted or corrected.

The multiplex CRISPR/Cas9-Based System can be used in a method of multiplex modulation of gene expression in a cell. The method includes contacting a cell with the modified lentiviral vector. The method may include modulating the gene expression levels of at least one gene. The gene expression of the at least one target gene is modulated when gene expression levels of the at least one target gene are increased or decreased compared to normal gene expression levels for the at least one target gene. The gene expression levels may be RNA or protein levels.

In some embodiments, it is also contemplated herein that the expression of multiple genes is modulated by introducing multiple, orthogonal Cas with multiple guide RNAs (e.g., multiplex modulation of gene expression or “orthogonal dCas9 systems”). For example, different Cas proteins or Cas9 proteins. One of skill in the art will appreciate that the plurality of guide RNAs should be designed to minimize off-target effects or interaction of the RNAs with one another. Orthogonal dCas9 systems permit the simultaneous activation of certain desired genes with repression of other desired genes. For example, a plurality of orthogonal Cas proteins (e.g., Cas9 proteins) derived from a combination of bacterial species e.g., S. pyogenes, N. meninigitidis, S. thermophilus and T. denticola can be used in combination as described in e.g., Esvelt, K et al. Nature Methods 10(11):1116-1121 (2013), which is herein incorporated by reference in its entirety. In some embodiments, a plurality of nucleic acid sequences encoding a plurality of guide RNAs are present on the same vector. Further, each dCas9 can be paired with a discrete inducible system, which can allow for independent control of activation and/or repression of the desired genes. In addition, this inducible orthogonal dCas9 system can also permit regulation of gene expression in a temporal manner (see e.g., Gao et al. Nature Methods Complex transcriptional modulation with orthogonal and inducible dCas9 regulators (2016)).

B. Homology-Directed Repair Templates

In some embodiments, a homology-directed recombination template or “repair” template is also provided in the ceDNA vector, e.g., as the donor sequence and/or part of the donor sequence. It is contemplated herein that a homology directed repair template can be used to repair a gene sequence or to insert a new sequence, for example, to manufacture a therapeutic protein. In some embodiments, a repair template is designed to serve as a template in homologous recombination, such as within or near a target sequence nicked or cleaved by a nuclease described herein, e.g., an RNA-guided endonuclease, such as a CRISPR enzyme as a part of a CRISPR complex, or ZFN or TALE. A template polynucleotide can be of any suitable length, such as about or more than about 10, 15, 20, 25, 50, 75, 100, 150, 200, 500, 1000, or more nucleotides in length. In some embodiments, the template polynucleotide is complementary to a portion of a polynucleotide comprising a target sequence in the host cell genome. When optimally aligned, a template polynucleotide can overlap with one or more nucleotides of a target sequence (e.g., about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence. In one embodiment, the homology arms of the repair template are directional (i.e., not identical and therefore bind to the sequence in a particular orientation). In some embodiments, two or more HDR templates are provided to repair a single gene in a cell, or two different genes in a cell. In some embodiments, multiple copies of at least one template are provided to a cell.

In some embodiments, the template sequence can be substantially identical to a portion of an endogenous target gene sequence but comprises at least one nucleotide change. In some embodiments, the repair of the cleaved target nucleic acid molecule can result in, for example, (i) one or more nucleotide changes in an RNA expressed from the target gene, (ii) altered expression level of the target gene, (iii) gene knockdown, (iv) gene knockout, (v) restored gene function, or (vi) gene knockout and simultaneous insertion of a gene. As will be readily appreciated by one of skill in the art the repair of the cleaved target nucleic acid molecule with the template can result in a change in an exon sequence, an intron sequence, a regulatory sequence, a transcriptional control sequence, a translational control sequence, a splicing site, or a non-coding sequence of the target gene. In other embodiments, the template sequence can comprise an exogenous sequence which can result in a gene-knock-in. Integration of the exogenous sequence can result in a gene knock-out.

In certain embodiments, the donor sequence is in a capsid-free ceDNA vector also including one or more integration elements such as a 5′ homology arm, and/or a 3′ homology arm. At a minimum in certain such embodiments, ceDNA comprises, from 5′ to 3′, a 5′ HDR arm, a donor sequence, a 3′ HDR arm, and at least one ITR, wherein the at least one ITR is upstream of the 5′ HDR arm or downstream of the 3′ HDR arm. In certain embodiments, the donor sequence (such as, but not limited to, Factor IX or Factor VIII (or e.g., any other therapeutic protein of interest) is a nucleotide sequence to be inserted into the chromosome of a host cell. In certain embodiments, the donor sequence is not originally present in the host cell or may be foreign to the host cell. In certain embodiments, the donor sequence is an endogenous sequence present at a site other than the predetermined target site. In certain embodiments, the donor sequence is an endogenous sequence similar to that of the pre-determined target site (e.g., replaces an existing erroneous sequence). In certain embodiments, the donor sequence is a sequence endogenous to the host cell, but which is present at a site other than the predetermined target site. In some embodiments, the donor sequence is a coding sequence or non-coding sequence. In some embodiments, the donor sequence is a mutant locus of a gene. In certain embodiments, the donor sequence may be an exogenous gene to be inserted into the chromosome, a modified sequence that replaces the endogenous sequence at the target site, a regulatory element, a tag or a coding sequence encoding a reporter protein and/or RNA. In some embodiments, the donor sequence may be inserted in frame into the coding sequence of a target gene for expression of a fusion protein. In certain embodiments, the donor sequence is not an entire ORF (coding/donor sequence), but just a corrective portion of DNA that is meant to replace a desired target. In certain embodiments, the donor sequence is inserted in-frame behind an endogenous promoter such that the donor sequence is regulated similarly to the naturally-occurring sequence.

In certain embodiments, the donor sequence may optionally include a promoter therein as described above in order to drive a coding sequence. Such embodiments may further include a poly-A tail within the donor sequence to facilitate expression.

In certain embodiments, the donor sequence may be a predetermined size, or sized by one of ordinary skill in the art. In certain embodiments, the donor sequence may be at least or about any of 10 base pairs, 15 base pairs, 20 base pairs, 25 base pairs, 50 base pairs, 60 base pairs, 75 base pairs, 100 base pairs, at least 150 base pairs, 200 base pairs, 300 base pairs, 500 base pairs, 800 base pairs, 1000 base pairs, 1,500 base pairs, 2,000 base pairs, 2500 base pairs, 3000 base pairs, 4000 base pairs, 4500 base pairs, and 5,000 base pairs in length or about 1 base pair to about 10 base pairs, or about 10 base pairs to about 50 base pairs, or between about 50 base pairs to about 100 base pairs, or between about 100 base pairs to about 500 base pairs, or between about 500 base pairs to about 5,000 base pairs in length. In certain embodiments, the donor sequence includes only 1 base pair to repair a single mutated nucleotide in the genome.

Non-limiting examples of suitable donor sequence(s) for use in accordance with the present disclosure include a promoter-less coding sequence corresponding to one or more disease-related sequences having at least 60%, more preferably at least 65%, more preferably at least 70%, more preferably at least 75%, more preferably at least 80%, more preferably at least 85%, even more preferably at least 90%, and most preferably at least 95% sequence identity to one of the disease-related molecules described herein. In one embodiment, the coding sequence has at least 60%, more preferably at least 65%, more preferably at least 70%, more preferably at least 75%, more preferably at least 80%, more preferably at least 85%, even more preferably at least 90%, and most preferably at least 95% sequence identity to SEQ ID NO: 825 or a donor sequence consisting of SEQ ID NO: 825. In certain embodiments, such as where the sequence is added rather than replaced, a promoter can be provided.

For integration of the donor sequence into the host cell genome, the ceDNA vector may rely on the polynucleotide sequence encoding the donor sequence or any other element of the vector for integration into the genome by homologous recombination such as the 5′ and 3′ homology arms shown therein (see e.g., FIG. 8). For example, the ceDNA vector may contain nucleotides encoding 5′ and 3′ homology arms for directing integration by homologous recombination into the genome of the host cell at a precise location(s) in the chromosome(s). To increase the likelihood of integration at a precise location, the 5′ and 3′ homology arms may include a sufficient number of nucleic acids, such as 50 to 5,000 base pairs, or 100 to 5,000 base pairs, or 500 to 5,000 base pairs, which have a high degree of sequence identity or homology to the corresponding target sequence to enhance the probability of homologous recombination. The 5′ and 3′ homology arms may be any sequence that is homologous with the target sequence in the genome of the host cell. Furthermore, the 5′ and 3′ homology arms may be non-encoding or encoding nucleotide sequences. In certain embodiments, the homology between the 5′ homology arm and the corresponding sequence on the chromosome is at least any of 80%, 85%, 90%, 95%, 97%, 98%, 99%, or 100%. In certain embodiments, the homology between the 3′ homology arm and the corresponding sequence on the chromosome is at least any of 80%, 85%, 90%, 95%, 97%, 98%, 99%, or 100%. In certain embodiments, the 5′ and/or 3′ homology arms can be homologous to a sequence immediately upstream and/or downstream of the integration or DNA cleavage site on the chromosome. Alternatively, the 5′ and/or 3′ homology arms can be homologous to a sequence that is distant from the integration or DNA cleavage site, such as at least 1, 2, 5, 10, 15, 20, 25, 30, 50, 100, 200, 300, 400, or 500 bp away from the integration or DNA cleavage site, or partially or completely overlapping with the DNA cleavage site. In certain embodiments, the 3′ homology arm of the nucleotide sequence is proximal to the altered ITR.

In certain embodiments, the efficiency of integration of the donor sequence is improved by extraction of the cassette comprising the donor sequence from the ceDNA vector prior to integration. In one nonlimiting example, a specific restriction site may be engineered 5′ to the 5′ homology arm, 3′ to the 3′ homology arm, or both. If such a restriction site is present with respect to both homology arms, then the restriction site may be the same or different between the two homology arms. When the ceDNA vector is cleaved with the one or more restriction endonucleases specific for the engineered restriction site(s), the resulting cassette comprises the 5′ homology arm-donor sequence-3′ homology arm, and can be more readily recombined with the desired genomic locus. It will be appreciated by one of ordinary skill in the art that this cleaved cassette may additionally comprise other elements such as, but not limited to, one or more of the following: a regulatory region, a nuclease, and an additional donor sequence. In certain aspects, the ceDNA vector itself may encode the restriction endonuclease such that upon delivery of the ceDNA vector to the nucleus the restriction endonuclease is expressed and able to cleave the vector. In certain aspects, the restriction endonuclease is encoded on a second ceDNA vector which is separately delivered. In certain aspects, the restriction endonuclease is introduced to the nucleus by a non-ceDNA-based means of delivery. In certain embodiments, the restriction endonuclease is introduced after the ceDNA vector is delivered to the nucleus. In certain embodiments, the restriction endonuclease and the ceDNA vector are transported to the nucleus simultaneously. In certain embodiments, the restriction endonuclease is already present upon introduction of the ceDNA vector.

In certain embodiments, the donor sequence is foreign to the 5′ homology arm or 3′ homology arm. In certain embodiments, the donor sequence is not endogenously found between the sequences comprising the 5′ homology arm and 3′ homology arm. In certain embodiments, the donor sequence is not endogenous to the native sequence comprising the 5′ homology arm or the 3′ homology arm. In certain embodiments, the 5′ homology arm is homologous to a nucleotide sequence upstream of a nuclease cleavage site on a chromosome. In certain embodiments, the 3′ homology arm is homologous to a nucleotide sequence downstream of a nuclease cleavage site on a chromosome. In certain embodiments, the 5′ homology arm or the 3′ homology arm are proximal to the at least one altered ITR. In certain embodiments, the 5′ homology arm or the 3′ homology arm are about 250 to 2000 bp.

Non-limiting examples of suitable 5′ homology arms for use in accordance with the present disclosure, and in particular for use in gene editing of liver cells or tissue, include a 5′ albumin homology arm having at least 60%, more preferably at least 65%, more preferably at least 70%, more preferably at least 75%, more preferably at least 80%, more preferably at least 85%, even more preferably at least 90%, and most preferably at least 95% sequence identity to a suitable segment within SEQ ID NO: 823 or SEQ ID NO: 826 or a 5′ homology arm consisting of a suitable segment within SEQ ID NO: 823 or a suitable segment within SEQ ID NO: 826. Such segments can be all of the respective sequences.

Non-limiting examples of suitable 3′ homology arms for use in accordance with the present disclosure include a 3′ albumin homology arm having at least 60%, more preferably at least 65%, more preferably at least 70%, more preferably at least 75%, more preferably at least 80%, more preferably at least 85%, even more preferably at least 90%, and most preferably at least 95% sequence identity to a suitable segment within SEQ ID NO: 824 or SEQ ID NO:14 827 or a 3′ homology arm consisting of a suitable segment within SEQ ID NO: 824 or SEQ ID NO: 827. Such segments can be all of the respective sequences.

In one embodiment, gene editing ceDNA vectors that comprise 5′- and 3′ homology arms flanking a donor sequence, as described herein, can be administered in conjunction with another vector (e.g., an additional ceDNA vector, a lentiviral vector, a viral vector, or a plasmid) that encodes a Cas nickase (nCas; e.g., Cas9 nickase). It is contemplated herein that such an nCas enzyme is used in conjunction with a guide RNA that comprises homology to a ceDNA vector as described herein and can be used, for example, to release physically constrained sequences or to provide torsional release. Releasing physically constrained sequences can, for example, “unwind” the ceDNA vector such that a homology directed repair (HDR) template homology arm(s) within the ceDNA vector are exposed for interaction with the genomic sequence. In addition, it is contemplated herein that such a system can be used to deactivate ceDNA vectors, if necessary. It will be understood by one of skill in the art that a Cas enzyme that induces a double-stranded break in the ceDNA vector would be a stronger deactivator of such ceDNA vectors. In one embodiment, the guide RNA comprises homology to a sequence inserted into the ceDNA vector such as a sequence encoding a nuclease or the donor sequence or template. In another embodiment, the guide RNA comprises homology to an inverted terminal repeat (ITR) or the homology/insertion elements of the ceDNA vector. In some embodiments, a ceDNA vector as described herein comprises an ITR on each of the 5′ and 3′ ends, thus a guide RNA with homology to the ITRs will produce nicking of the one or more ITRs substantially equally. In some embodiments, a guide RNA has homology to some portion of the ceDNA vector and the donor sequence or template (e.g., to assist with unwinding the ceDNA vector). It is also contemplated herein that there are certain sites on the ceDNA vectors that when nicked may result in the inability of the ceDNA vector to be retained in the nucleus. One of ordinary skill in the art can readily identify such sequences and can thus avoid engineering guide RNAs to such sequence regions. Alternatively, modifying the subcellular localization of a ceDNA vector to a region outside the nuclease by using a guide RNA that nicks sequences responsible for nuclear localization can be used as a method of deactivating the ceDNA vector, if necessary or desired.

In certain embodiments, other integration strategies and components are suitable for use in accordance with ceDNA vectors of the present disclosure. For example, although not shown in FIGS. 1A-1G or FIG. 8 or FIG. 9, in one embodiment, a ceDNA vector in accordance with the present disclosure may include an expression cassette flanked by ribosomal DNA (rDNA) sequences capable of homologous recombination into genomic rDNA. Similar strategies have been performed, for example, in Lisowski, et al., Ribosomal DNA Integrating rAAV-rDNA Vectors Allow for Stable Transgene Expression, The American Society of Gene and Cell Therapy, 18 Sep. 2012 (herein incorporated by reference in its entirety) where rAAV-rDNA vectors were demonstrated. In certain embodiments, delivery of ceDNA-rDNA vectors may integrate into the genomic rDNA locus with increased frequency, where the integrations are specific to the rDNA locus. Moreover, a ceDNA-rDNA vector containing a human factor IX (hFIX) or human Factor VIII expression cassette increases therapeutic levels of serum hFIX or human Factor VIII. Because of the relative safety of integration in the rDNA locus, ceDNA-rDNA vectors expand the usage of ceDNA for therapeutics requiring long-term gene transfer into dividing cells.

In one embodiment, a promoterless ceDNA vector is contemplated for delivery of a homology repair template (e.g., a repair sequence with two flanking homology arms) but does not comprise nucleic acid sequences encoding a nuclease or guide RNA.

The methods and compositions described herein can be used in methods comprising homology recombination, for example, as described in Rouet et al. Proc Natl Acad Sci 91:6064-6068 (1994); Chu et al. Nat Biotechnol 33:543-548 (2015); Richardson et al. Nat Biotechnol 33:339-344 (2016); Komor et al. Nature 533:420-424 (2016); the contents of each of which are incorporated by reference herein in their entirety.

The methods and compositions described herein can be used in methods comprising homology recombination, for example, as described in Rouet et al. Proc Natl Acad Sci 91:6064-6068 (1994); Chu et al. Nat Biotechnol 33:543-548 (2015); Richardson et al. Nat Biotechnol 33:339-344 (2016); Komor et al. Nature 533:420-424 (2016); the contents of each of which are incorporated by reference herein in their entirety.

C. Guide RNAs (gRNAs)

In general, a guide sequence is any polynucleotide sequence having sufficient complementarity with a target polynucleotide sequence to hybridize with the target sequence and direct sequence-specific targeting of an RNA-guided endonuclease complex to the selected genomic target sequence. In some embodiments, a guide RNA binds and e.g., a Cas protein can form a ribonucleoprotein (RNP), for example, a CRISPR/Cas complex.

In some embodiments, the guide RNA (gRNA) sequence comprises a targeting sequence that directs the gRNA sequence to a desired site in the genome, fused to a crRNA and/or tracrRNA sequence that permit association of the guide sequence with the RNA-guided endonuclease. In some embodiments, the degree of complementarity between a guide sequence and its corresponding target sequence, when optimally aligned using a suitable alignment algorithm, is at least 50%, 60%, 75%, 80%, 85%, 90%, 95%, 97.5%, 99%, or more. Optimal alignment can be determined with the use of any suitable algorithm for aligning sequences, such as the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP, and Maq. In some embodiments, a guide sequence is 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. It is contemplated herein that the targeting sequence of the guide RNA and the target sequence on the target nucleic acid molecule can comprise 1, 2, 3, 4, 5, 6, 7, 8, 9, or 10 mismatches. In some embodiments, the guide RNA sequence comprises a palindromic sequence, for example, the self-targeting sequence comprises a palindrome. The targeting sequence of the guide RNA is typically 19-21 base pairs long and directly precedes the hairpin that binds the entire guide RNA (targeting sequence+hairpin) to a Cas such as Cas9. Where a palindromic sequence is employed as the self-targeting sequence of the guide RNA, the inverted repeat element can be e.g., 9, 10, 11, 12, or more nucleotides in length. Where the targeting sequence of the guide RNA is most often 19-21 bp, a palindromic inverted repeat element of 9 or 10 nucleotides provides a targeting sequence of desirable length. The Cas9-guide RNA hairpin complex can then recognize and cut any nucleotide sequence (DNA or RNA) e.g., a DNA sequence that matches the 19-21 base pair sequence and is followed by a “PAM” sequence e.g., NGG or NGA, or other PAM.

The ability of a guide sequence to direct sequence-specific binding of an RNA-guided endonuclease complex to a target sequence can be assessed by any suitable assay. For example, the components of an RNA-guided endonuclease system sufficient to form an RNA-guided endonuclease complex can be provided to a host cell having the corresponding target sequence, such as by transfection with vectors encoding the components of the RNA-guided endonuclease sequence, followed by an assessment of preferential cleavage within the target sequence, such as by Surveyor assay (Transgenomic™, New Haven, Conn.). Similarly, cleavage of a target polynucleotide sequence can be evaluated in a test tube by providing the target sequence, components of an RNA-guided endonuclease complex, including the guide sequence to be tested and a control guide sequence different from the test guide sequence, and comparing binding or rate of cleavage at the target sequence between the test and control guide sequence reactions. One of ordinary skill in the art will appreciate that other assays can also be used to test gRNA sequences.

A guide sequence can be selected to target any target sequence. In some embodiments, the target sequence is a sequence within a genome of a cell. In some embodiments, the target sequence is the sequence encoding a first guide RNA in a self-cloning plasmid, as described herein. Typically, the target sequence in the genome will include a protospacer adjacent (PAM) sequence for binding of the RNA-guided endonuclease. It will be appreciated by one of skill in the art that the PAM sequence and the RNA-guided endonuclease should be selected from the same (bacterial) species to permit proper association of the endonuclease with the targeting sequence. For example, the PAM sequence for CAS9 is different than the PAM sequence for cpFl. Design is based on the appropriate PAM sequence. To prevent degradation of the guide RNA, the sequence of the guide RNA should not contain the PAM sequence. In some embodiments, the length of the targeting sequence in the guide RNA is 12 nucleotides; in other embodiments, the length of the targeting sequence in the guide RNA is 13, 14, 15, 16, 17, 18, 19, 20, 21, 22, 23, 24, 25, 26, 27, 28, 29, 30, 35 or 40 nucleotides. The guide RNA can be complementary to either strand of the targeted DNA sequence. In some embodiments, when modifying the genome to include an insertion or deletion, the gRNA can be targeted closer to the N-terminus of a protein coding region.

It will be appreciated by one of skill in the art that for the purposes of targeted cleavage by an RNA-guided endonuclease, target sequences that are unique in the genome are preferred over target sequences that occur more than once in the genome. Bioinformatics software can be used to predict and minimize off-target effects of a guide RNA (see e.g., Naito et al. “CRISPRdirect: software for designing CRISPR/Cas guide RNA with reduced off-target sites” Bioinformatics (2014), epub; Heigwer, F., et al. “E-CRISP: fast CRISPR target site identification” Nat. Methods 11, 122-123 (2014); Bae et al. “Cas-OFFinder: a fast and versatile algorithm that searches for potential off-target sites of Cas9 RNA-guided endonucleases” Bioinformatics 30(10):1473-1475 (2014); Aach et al. “CasFinder: Flexible algorithm for identifying specific Cas9 targets in genomes” BioRxiv (2014), among others).

For the S. pyogenes Cas9, a unique target sequence in a genome can include a Cas9 target site of the form MMMMMMMMNNNNNNNNNNNNXGG (SEQ ID NO: 590) where NNNNNNNNNNNNXGG (SEQ ID NO: 591) (N is A, G, T, or C; and X can be any nucleotide) has a single occurrence in the genome. A unique target sequence in a genome can include an S. pyogenes Cas9 target site of the form MMMMMMMMMNNNNNNNNNNNXGG (SEQ ID NO: 592) where NNNNNNNNNNNXGG (SEQ ID NO: 593) (N is A, G, T, or C; and X can be any nucleotide) has a single occurrence in the genome. For the S. thermophilus CRISPR1 Cas9, a unique target sequence in a genome can include a Cas9 target site of the form MMMMMMMMNNNNNNNNNNNNXXAGAAW (SEQ ID NO: 594) where NNNNNNNNNNNNXXAGAAW (SEQ ID NO: 595) (N is A, G, T, or C; X can be any nucleotide; and W is A or T) has a single occurrence in the genome. A unique target sequence in a genome can include an S. thermophilus CRISPR 1 Cas9 target site of the form MMMMMMMMMNNNNNNNNNNNXXAGAAW (SEQ ID NO: 596) where NNNNNNNNNNNXXAGAAW (SEQ ID NO: 597) (N is A, G, T, or C; X can be any nucleotide; and W is A or T) has a single occurrence in the genome. For the S. pyogenes Cas9, a unique target sequence in a genome can include a Cas9 target site of the form MMMMMMMMNNNNNNNNNNNNXGGXG (SEQ ID NO: 598) where NNNNNNNNNNNNXGGXG (SEQ ID NO: 599) (N is A, G, T, or C; and X can be any nucleotide) has a single occurrence in the genome. A unique target sequence in a genome can include an S. pyogenes Cas9 target site of the form MMMMMMMMMNNNNNNNNNNNXGGXG (SEQ ID NO: 600) where NNNNNNNNNNNXGGXG (SEQ ID NO: 601) (N is A, G, T, or C; and X can be any nucleotide) has a single occurrence in the genome. In each of these sequences “M” may be A, G, T, or C, and need not be considered in identifying a sequence as unique.

In general, a “crRNA/tracrRNA fusion sequence,” as that term is used herein refers to a nucleic acid sequence that is fused to a unique targeting sequence and that functions to permit formation of a complex comprising the guide RNA and the RNA-guided endonuclease. Such sequences can be modeled after CRISPR RNA (crRNA) sequences in prokaryotes, which comprise (i) a variable sequence termed a “protospacer” that corresponds to the target sequence as described herein, and (ii) a CRISPR repeat. Similarly, the tracrRNA (“transactivating CRISPR RNA”) portion of the fusion can be designed to comprise a secondary structure similar to the tracrRNA sequences in prokaryotes (e.g., a hairpin), to permit formation of the endonuclease complex. In some embodiments, the fusion has sufficient complementarity with a tracrRNA sequence to promote one or more of: (1) excision of a guide sequence flanked by tracrRNA sequences in a cell containing the corresponding tracr sequence; and (2) formation of an endonuclease complex at a target sequence, wherein the complex comprises the crRNA sequence hybridized to the tracrRNA sequence. In general, degree of complementarity is with reference to the optimal alignment of the crRNA sequence and tracrRNA sequence, along the length of the shorter of the two sequences. Optimal alignment can be determined by any suitable alignment algorithm, and can further account for secondary structures, such as self-complementarity within either the tracrRNA sequence or crRNA sequence. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracrRNA sequence is at least 5, 6, 7, 8, 9, 10, 11, 12, 13, 14, 15, 16, 17, 18, 19, 20, 25, 30, 40, 50, 60, 70, 80, 90, 100, or more nucleotides in length (e.g., 70-80, 70-75, 75-80 nucleotides in length). In one embodiment, the crRNA is less than 60, less than 50, less than 40, less than 30, or less than 20 nucleotides in length. In other embodiments, the crRNA is 30-50 nucleotides in length; in other embodiments the crRNA is 30-50, 35-50, 40-50, 40-45, 45-50 or 50-55 nucleotides in length. In some embodiments, the crRNA sequence and tracrRNA sequence are contained within a single transcript, such that hybridization between the two produces a transcript having a secondary structure, such as a hairpin. In some embodiments, the loop forming sequences for use in hairpin structures are four nucleotides in length, for example, the sequence GAAA. However, longer or shorter loop sequences can be used, as can alternative sequences. The sequences preferably include a nucleotide triplet (for example, AAA), and an additional nucleotide (for example C or G). Examples of loop forming sequences include CAAA and AAAG. In one embodiment, the transcript or transcribed gRNA sequence comprises at least one hairpin. In one embodiment, the transcript or transcribed polynucleotide sequence has at least two or more hairpins. In other embodiments, the transcript has two, three, four or five hairpins. In a further embodiment, the transcript has at most five hairpins. In some embodiments, the single transcript further includes a transcription termination sequence, such as a polyT sequence, for example six T nucleotides. Non-limiting examples of single polynucleotides comprising a guide sequence, a crRNA sequence, and a tracr sequence are as follows (listed 5′ to 3′), where “N” represents a base of a guide sequence, the first block of lower case letters represent the crRNA sequence, and the second block of lower case letters represent the tracr sequence, and the final poly-T sequence represents the transcription terminator: (i) NNNNNNNNNNNNNNNNNNNNgtttttgtactctcaagatttaGAAAtaaatcttgcagaagctacaaagataaggctt catgccgaaatcaacaccctgtcattttatggcagggtgttttcgttatttaaTTTTTT (SEQ ID NO: 602); (ii) NNNNNNNNNNNNNNNNNNNNgtttttgtactctcaGAAAthcagaagctacaaagataaggcttcatgccgaaatca acaccctgtcattttatggcagggtgttttcgttatttaaTTTTTT (SEQ ID NO: 603); (iii) NNNNNNNNNNNNNNNNNNNNgtttttgtactctcaGAAAtgcagaagctacaaagataaggcttcatgccgaaatca acaccctgtcattttatggcagggtgtTTTTTT (SEQ ID NO: 604); (iv) NNNNNNNNNNNNNNNNNNNNgttttagagctaGAAAtagcaagttaaaataaggctagtccgttatcaacttgaaaa agtggcaccgagtcggtgcTTTTTT (SEQ ID NO: 605); (v) NNNNNNNNNNNNNNNNNNNNgttttagagctaGAAATAGcaagttaaaataaggctagtccgttatcaacttgaa aaagtTTTTTTT (SEQ ID NO: 606); and (vi) NNNNNNNNNNNNNNNNNNNNgttttagagctagAAATAGcaagttaaaataaggctagtccgttatcaTTTTT TTT (SEQ ID NO: 607). In some embodiments, sequences (i) to (iii) are used in combination with Cas9 from S. thermophilus CRISPR1. In some embodiments, sequences (iv) to (vi) are used in combination with Cas9 from S. pyogenes. In some embodiments, the tracrRNA sequence is a separate transcript from a transcript comprising the crRNA sequence.

In some embodiments, a guide RNA can comprise two RNA molecules and is referred to herein as a “dual guide RNA” or “dgRNA.” In some embodiments, the dgRNA may comprise a first RNA molecule comprising a crRNA, and a second RNA molecule comprising a tracrRNA. The first and second RNA molecules may form a RNA duplex via the base pairing between the flagpole on the crRNA and the tracrRNA. When using a dgRNA, the flagpole need not have an upper limit with respect to length.

In other embodiments, a guide RNA can comprise a single RNA molecule and is referred to herein as a “single guide RNA” or “sgRNA.” In some embodiments, the sgRNA can comprise a crRNA covalently linked to a tracrRNA. In some embodiments, the crRNA and tracrRNA can be covalently linked via a linker. In some embodiments, the sgRNA can comprise a stem-loop structure via the base-pairing between the flagpole on the crRNA and the tracrRNA. In some embodiments, a single-guide RNA is at least 50, at least 60, at least 70, at least 80, at least 90, at least 100, at least 110, at least 120 or more nucleotides in length (e.g., 75-120, 75-110, 75-100, 75-90, 75-80, 80-120, 80-110, 80-100, 80-90, 85-120, 85-110, 85-100, 85-90, 90-120, 90-110, 90-100, 100-120, 100-120 nucleotides in length). In some embodiments, a ceDNA vector or composition thereof comprises a nucleic acid that encodes at least 1 gRNA. For example, the second polynucleotide sequence may encode at least 1 gRNA, at least 2 gRNAs, at least 3 gRNAs, at least 4 gRNAs, at least 5 gRNAs, at least 6 gRNAs, at least 7 gRNAs, at least 8 gRNAs, at least 9 gRNAs, at least 10 gRNAs, at least 11 gRNA, at least 12 gRNAs, at least 13 gRNAs, at least 14 gRNAs, at least 15 gRNAs, at least 16 gRNAs, at least 17 gRNAs, at least 18 gRNAs, at least 19 gRNAs, at least 20 gRNAs, at least 25 gRNA, at least 30 gRNAs, at least 35 gRNAs, at least 40 gRNAs, at least 45 gRNAs, or at least 50 gRNAs. The second polynucleotide sequence may encode between 1 gRNA and 50 gRNAs, between 1 gRNA and 45 gRNAs, between 1 gRNA and 40 gRNAs, between 1 gRNA and 35 gRNAs, between 1 gRNA and 30 gRNAs, between 1 gRNA and 25 different gRNAs, between 1 gRNA and 20 gRNAs, between 1 gRNA and 16 gRNAs, between 1 gRNA and 8 different gRNAs, between 4 different gRNAs and 50 different gRNAs, between 4 different gRNAs and 45 different gRNAs, between 4 different gRNAs and 40 different gRNAs, between 4 different gRNAs and 35 different gRNAs, between 4 different gRNAs and 30 different gRNAs, between 4 different gRNAs and 25 different gRNAs, between 4 different gRNAs and 20 different gRNAs, between 4 different gRNAs and 16 different gRNAs, between 4 different gRNAs and 8 different gRNAs, between 8 different gRNAs and 50 different gRNAs, between 8 different gRNAs and 45 different gRNAs, between 8 different gRNAs and 40 different gRNAs, between 8 different gRNAs and 35 different gRNAs, between 8 different gRNAs and 30 different gRNAs, between 8 different gRNAs and 25 different gRNAs, between 8 different gRNAs and 20 different gRNAs, between 8 different gRNAs and 16 different gRNAs, between 16 different gRNAs and 50 different gRNAs, between 16 different gRNAs and 45 different gRNAs, between 16 different gRNAs and 40 different gRNAs, between 16 different gRNAs and 35 different gRNAs, between 16 different gRNAs and 30 different gRNAs, between 16 different gRNAs and 25 different gRNAs, or between 16 different gRNAs and 20 different gRNAs. Each of the polynucleotide sequences encoding the different gRNAs may be operably linked to a promoter. The promoters that are operably linked to the different gRNAs may be the same promoter. The promoters that are operably linked to the different gRNAs may be different promoters. The promoter may be a constitutive promoter, an inducible promoter, a repressible promoter, or a regulatable promoter.

In some experiments, the guide RNAs will target known ZFN sequence targeted regions successful for knock-ins, or knock-out deletions, or for correction of defective genes. Multiple sgRNA sequences that bind known ZFN target regions have been designed and are described in Tables 1-2 of US patent publication 2015/0056705, which is herein incorporated by reference in its entirety, and include for example gRNA sequences for human beta-globin, human, BCLIIA, human KLF1, Human CCR5, Human CXCR4, PPP1R12C, mouse and human HPRT, human albumin, human factor IX, human factor VIII, human LRRK2, human Htt, human RH, CFTR, TRAC, TRBC, human PD1, human CTLA-4, HLA c11, HLA A2, HLA A3, HLA B, HLA C, HLA c1. II DBp2. DRA, Tap 1 and 2. Tapasin, DMD, RFX5, etc.,)

Modified nucleosides or nucleotides can be present in a guide RNA or mRNA as described herein. An mRNA encoding a guide RNA or a DNA endonuclease (e.g., an RNA-guided nuclease) can comprise one or more modified nucleosides or nucleotides; such mRNAs are called “modified” to describe the presence of one or more non-naturally and/or naturally occurring components or configurations that are used instead of or in addition to the canonical A, G, C, and U residues. In some embodiments, a modified RNA is synthesized with a non-canonical nucleoside or nucleotide, here called “modified.” Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar and/or backbone modification); and (vii) modification or replacement of the sugar (an exemplary sugar modification). Unmodified nucleic acids can be prone to degradation by, e.g., cellular nucleases. For example, nucleases can hydrolyze nucleic acid phosphodiester bonds. Accordingly, in one aspect the guide RNAs described herein can contain one or more modified nucleosides or nucleotides, e.g., to introduce stability toward nucleases. In certain embodiments, the mRNAs described herein can contain one or more modified nucleosides or nucleotides, e.g., to introduce stability toward nucleases. In one embodiment, the modification includes 2′-O-methyl nucleotides. In other embodiments, the modification comprises phosphorothioate (PS) linkages.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens. The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxy methyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Modified nucleosides and nucleotides can include one or more modifications to the sugar group, i.e. at sugar modification. For example, the 2′ hydroxyl group (OH) can be modified, e.g., replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion. Examples of 2′ hydroxyl group modifications can include alkoxy or aryloxy (OR, wherein “R” can be, e.g., alkyl, cycloalkyl, aryl, aralkyl, heteroaryl or a sugar); poly ethylene glycols (PEG), 0(CH2CH20)nCH2CH2OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the 2′ hydroxyl group modification can be 2′-O-Me. In some embodiments, the 2′ hydroxyl group modification can be a 2′-fluoro modification, which replaces the 2′ hydroxyl group with a fluoride. In some embodiments, the 2′ hydroxyl group modification can include “locked” nucleic acids (LNA) in which the 2′ hydroxyl can be connected, e.g., by a Ci-6 alkylene or Ci-6 heteroalkylene bridge, to the 4′ carbon of the same ribose sugar, where exemplary bridges can include methylene, propylene, ether, or amino bridges; O-amino (wherein amino can be, e.g., NH2; alkylamino, dialkylamino, heterocyclyl, arylamino, diarylamino, heteroarylamino, or diheteroarylamino, ethylenediamine, or polyamino) and aminoalkoxy, 0(CH2)n-amino, (wherein amino can be, e.g., NH2; alkylamino, dialkylamino, heterocyclyl, arylamino, diarylamino, heteroarylamino, or diheteroarylamino, ethylenediamine, or polyamino). In some embodiments, the 2′ hydroxyl group modification can include “unlocked” nucleic acids (UNA) in which the ribose ring lacks the C2′-C3′ bond. In some embodiments, the 2′ hydroxyl group modification can include the methoxyethyl group (MOE), (OCH2CH2OCH3, e.g., a PEG derivative).

The term “Deoxy” 2′ modifications can include hydrogen (i.e. deoxyribose sugars, e.g., at the overhang portions of partially dsRNA); halo (e.g., bromo, chloro, fluoro, or iodo); amino (wherein amino can be, e.g., —NH2, alkylamino, dialkylamino, heterocyclyl, arylamino, diarylamino, heteroarylamino, diheteroarylamino, or amino acid); NH(CH2CH2NH)nCH2CH2-amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein. The sugar modification can comprise a sugar group which can also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form, e.g. L-nucleosides.

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In embodiments employing a dual guide RNA, each of the crRNA and the tracr RNA can contain modifications. Such modifications may be at one or both ends of the crRNA and/or tracr RNA. In certain embodiments comprising an sgRNA, one or more residues at one or both ends of the sgRNA may be chemically modified, or the entire sgRNA may be chemically modified. Certain embodiments comprise a 5′ end modification. Certain embodiments comprise a 3′ end modification. In certain embodiments, one or more or all of the nucleotides in single stranded overhang of a guide RNA molecule are deoxynucleotides. The modified mRNA can contain 5′ end and/or 3′ end modifications.

D. Regulatory Elements.

The ceDNA vectors for gene editing comprising an asymmetric ITR pair or symmetric ITR pair as defined herein, can be produced from expression constructs that further comprise a specific combination of cis-regulatory elements. The cis-regulatory elements include, but are not limited to, a promoter, a riboswitch, an insulator, a mir-regulatable element, a post-transcriptional regulatory element, a tissue- and cell type-specific promoter and an enhancer. In some embodiments, the ITR can act as the promoter for the transgene. In some embodiments, the ceDNA vector comprises additional components to regulate expression of the transgene, for example, regulatory switches as described herein, to regulate the expression of the transgene, or a kill switch, which can kill a cell comprising the ceDNA vector. Regulatory elements, including Regulatory Switches that can be used in the present invention are more fully discussed in PCT/US18/49996, which is incorporated herein in its entirety by reference.

In embodiments, the second nucleotide sequence includes a regulatory sequence, and a nucleotide sequence encoding a nuclease. In certain embodiments the gene regulatory sequence is operably linked to the nucleotide sequence encoding the nuclease. In certain embodiments, the regulatory sequence is suitable for controlling the expression of the nuclease in a host cell. In certain embodiments, the regulatory sequence includes a suitable promoter sequence, being able to direct transcription of a gene operably linked to the promoter sequence, such as a nucleotide sequence encoding the nuclease(s) of the present disclosure. In certain embodiments, the second nucleotide sequence includes an intron sequence linked to the 5′ terminus of the nucleotide sequence encoding the nuclease. In certain embodiments, an enhancer sequence is provided upstream of the promoter to increase the efficacy of the promoter. In certain embodiments, the regulatory sequence includes an enhancer and a promoter, wherein the second nucleotide sequence includes an intron sequence upstream of the nucleotide sequence encoding a nuclease, wherein the intron includes one or more nuclease cleavage site(s), and wherein the promoter is operably linked to the nucleotide sequence encoding the nuclease.

The ceDNA vectors can be produced from expression constructs that further comprise a specific combination of cis-regulatory elements such as WHP posttranscriptional regulatory element (WPRE) (e.g., SEQ ID NO: 8) and BGH polyA (SEQ ID NO: 9). Suitable expression cassettes for use in expression constructs are not limited by the packaging constraint imposed by the viral capsid.

(i). Promoters:

It will be appreciated by one of ordinary skill in the art that promoters used in the gene-editing ceDNA vectors of the invention should be tailored as appropriate for the specific sequences they are promoting. For example, a guide RNA may not require a promoter at all, since its function is to form a duplex with a specific target sequence on the native DNA to effect a recombination event. In contrast, a nuclease encoded by the ceDNA vector would benefit from a promoter so that it can be efficiently expressed from the vector—and, optionally, in a regulatable fashion.

Expression cassettes of the present invention include a promoter, which can influence overall expression levels as well as cell-specificity. For transgene expression, they can include a highly active virus-derived immediate early promoter. Expression cassettes can contain tissue-specific eukaryotic promoters to limit transgene expression to specific cell types and reduce toxic effects and immune responses resulting from unregulated, ectopic expression. In preferred embodiments, an expression cassette can contain a synthetic regulatory element, such as a CAG promoter (SEQ ID NO: 3). The CAG promoter comprises (i) the cytomegalovirus (CMV) early enhancer element, (ii) the promoter, the first exon and the first intron of chicken beta-actin gene, and (iii) the splice acceptor of the rabbit beta-globin gene. Alternatively, an expression cassette can contain an Alpha-1-antitrypsin (AAT) promoter (SEQ ID NO: 4 or SEQ ID NO: 74), a liver specific (LP1) promoter (SEQ ID NO: 5 or SEQ ID NO: 16), or a Human elongation factor-1 alpha (EF1a) promoter (e.g., SEQ ID NO: 6 or SEQ ID NO: 15). In some embodiments, the expression cassette includes one or more constitutive promoters, for example, a retroviral Rous sarcoma virus (RSV) LTR promoter (optionally with the RSV enhancer), or a cytomegalovirus (CMV) immediate early promoter (optionally with the CMV enhancer, e.g., SEQ ID NO: 22). Alternatively, an inducible promoter, a native promoter for a transgene, a tissue-specific promoter, or various promoters known in the art can be used.

Suitable promoters, including those described above, can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6, e.g., SEQ ID NO: 18) (Miyagishi et al., Nature Biotechnology 20, 497-500 (2002)), an enhanced U6 promoter (e.g., Xia et al., Nucleic Acids Res. 2003 Sep. 1; 31(17)), a human H1 promoter (H1) (e.g., SEQ ID NO: 19), a CAG promoter, a human alpha 1-antitypsin (HAAT) promoter (e.g., SEQ ID NO: 21), and the like. In certain embodiments, these promoters are altered at their downstream intron containing end to include one or more nuclease cleavage sites. In certain embodiments, the DNA containing the nuclease cleavage site(s) is foreign to the promoter DNA.

In one embodiment, the promoter used is the native promoter of the gene encoding the therapeutic protein. The promoters and other regulatory sequences for the respective genes encoding the therapeutic proteins are known and have been characterized. The promoter region used may further include one or more additional regulatory sequences (e.g., native), e.g., enhancers, (e.g. SEQ ID NO: 22 and SEQ ID NO: 23).

Non-limiting examples of suitable promoters for use in accordance with the present invention include the CAG promoter of, for example (SEQ ID NO: 3), the HAAT promoter (SEQ ID NO: 21), the human EF1-α promoter (SEQ ID NO: 6) or a fragment of the EFla promoter (SEQ ID NO: 15), IE2 promoter (e.g., SEQ ID NO: 20) and the rat EF1-α promoter (SEQ ID NO: 24).

(ii). Polyadenylation Sequences:

A sequence encoding a polyadenylation sequence can be included in the ceDNA vector to stabilize an mRNA expressed from the ceDNA vector, and to aid in nuclear export and translation. In one embodiment, the ceDNA vector does not include a polyadenylation sequence. In other embodiments, the vector includes at least 1, at least 2, at least 3, at least 4, at least 5, at least 10, at least 15, at least 20, at least 25, at least 30, at least 40, least 45, at least 50 or more adenine dinucleotides. In some embodiments, the polyadenylation sequence comprises about 43 nucleotides, about 40-50 nucleotides, about 40-55 nucleotides, about 45-50 nucleotides, about 35-50 nucleotides, or any range there between.

The expression cassettes can include a poly-adenylation sequence known in the art or a variation thereof, such as a naturally occurring sequence isolated from bovine BGHpA (e.g., SEQ ID NO: 74) or a virus SV40 pA (e.g., SEQ ID NO: 10), or a synthetic sequence (e.g., SEQ ID NO: 27). Some expression cassettes can also include SV40 late polyA signal upstream enhancer (USE) sequence. In some embodiments, the, USE can be used in combination with SV40 pA or heterologous poly-A signal.

The expression cassettes can also include a post-transcriptional element to increase the expression of a transgene. In some embodiments, Woodchuck Hepatitis Virus (WHP) posttranscriptional regulatory element (WPRE) (e.g., SEQ ID NO: 8) is used to increase the expression of a transgene. Other posttranscriptional processing elements such as the post-transcriptional element from the thymidine kinase gene of herpes simplex virus, or hepatitis B virus (HBV) can be used. Secretory sequences can be linked to the transgenes, e.g., VH-02 and VK-A26 sequences, e.g., SEQ ID NO: 25 and SEQ ID NO: 26.

(iii). Nuclear Localization Sequences

In some embodiments, the vector encoding an RNA guided endonuclease comprises one or more nuclear localization sequences (NLSs), for example, 1, 2, 3, 4, 5, 6, 7, 8, 9, 10, or more NLSs. In some embodiments, the one or more NLSs are located at or near the amino-terminus, at or near the carboxy-terminus, or a combination of these (e.g., one or more NLS at the amino-terminus and/or one or more NLS at the carboxy terminus). When more than one NLS is present, each can be selected independently of the others, such that a single NLS is present in more than one copy and/or in combination with one or more other NLSs present in one or more copies. Non-limiting examples of NLSs are shown in Table 6.

TABLE 6
Nuclear Localization Signals

| | | SEQ |
|---|---|---|
| | | ID |
| SOURCE | SEQUENCE | NO. |
| SV40 virus | PKKKRKV (encoded by | 573 |
| large | CCCAAGAAGAAGAGGAAGGTG; | |
| T-antigen | SEQ ID NO: 574) | |
| nucleoplasmin | KRPAATKKAGQAKKKK | 575 |
| c-myc | | |
| | PAAKRVKLD | 576 |
| | RQRRNELKRSP | 577 |
| hRNPA1 M9 | NQSSNFGPMKGGNFGGRSSGPY | 578 |
| | GGGGQYFAKPRNQGGY | |
| IBB domain | RMRIZFKNKGKDTAELRRRRVE | 579 |
| from | VSVELRKAKKDEQILKRRNV | |
| importin-alpha | | |
| myoma T protein | VSRKRPRP | 580 |
| | PPKKARED | 581 |
| human p53 | PQPKKKPL | 582 |
| mouse c-abl IV | SALIKKKKKMAP | 583 |
| influenza virus | DRLRR | 584 |
| NSI | | |
| | PKQKKRK | 585 |
| Hepatitis virus | RKLKKKIKKL | 586 |
| delta antigen | | |
| mouse Mxl | REKKKFLKRR | 587 |
| protein | | |
| human | KRKGDEVDGVDEVAKKKSKK | 588 |
| poly(ADP- | | |
| ribose) | | |
| polymerase | | |

E. Additional Components of Gene Editing Systems

The ceDNA vectors of the present disclosure may contain nucleotides that encode other components for gene editing. For example, to select for specific gene targeting events, a protective shRNA may be embedded in a microRNA and inserted into a recombinant ceDNA vector designed to integrate site-specifically into the highly active locus, such as an albumin locus. Such embodiments may provide a system for in vivo selection and expansion of gene-modified hepatocytes in any genetic background such as described in Nygaard et al., A universal system to select gene-modified hepatocytes in vivo, Gene Therapy, Jun. 8, 2016. The ceDNA vectors of the present disclosure may contain one or more selectable markers that permit selection of transformed, transfected, transduced, or the like cells. A selectable marker is a gene the product of which provides for biocide or viral resistance, resistance to heavy metals, prototrophy to auxotrophs, NeoR, and the like. In certain embodiments, positive selection markers are incorporated into the donor sequences such as NeoR. Negative selections markers may be incorporated downstream the donor sequences, for example a nucleic acid sequence HSV-tk encoding a negative selection marker may be incorporated into a nucleic acid construct downstream the donor sequence. Referring to FIG. 8, a transgene is optionally fused to a selection marker (NeoR) through a viral 2A peptide cleavage site (2A) flanked by 0.05 to 6 kb stretching homology arms. In certain embodiments, a negative selection marker such as HSV TK) and expressing unit that allows to control and select for successful correct site usage, may optionally be positioned outside the homology arms.

In embodiments, the ceDNA vector of the present disclosure may include a polyadenylation site upstream and proximate to the 5′ homology arm.

Referring to FIG. 9, a ceDNA vector in accordance with the present disclosure is shown including ceDNA specific ITR. The ceDNA vector includes a Pol III promoter driven (such as U6 and H1) sgRNA expressing unit with optional orientation with respect to the transcription direction. An sgRNA target sequence for a “double mutant nickase” is optionally provided to release torsion downstream of the 3′ homology arm close to the mutant ITR. Such embodiments increase annealing and promote HDR frequency.

In some embodiments, a nuclease comprised by a ceDNA vector described herein can be inactivated/diminished after gene editing. See for example, Example 6 (see also FIGS. 8, 9 and 13) herein.

F. Regulatory Switches

A molecular regulatory switch is one which generates a measurable change in state in response to a signal. Such regulatory switches can be usefully combined with the ceDNA vectors described herein to control the output of the ceDNA vector. In some embodiments, the ceDNA vector comprises a regulatory switch that serves to fine tune expression of the transgene. For example, it can serve as a biocontainment function of the ceDNA vector. In some embodiments, the switch is an “ON/OFF” switch that is designed to start or stop (i.e., shut down) expression of the gene of interest in the ceDNA in a controllable and regulatable fashion. In some embodiments, the switch can include a “kill switch” that can instruct the cell comprising the ceDNA vector to undergo cell programmed death once the switch is activated. Exemplary regulatory switches encompassed for use in a gene editing ceDNA to regulate the expression of a gene editing molecule (e.g., transgene, e.g., encoding an endonuclease, guide RNA, gDNA, RNA activator, or a donor sequence, are more fully discussed in PCT/US18/49996, which is incorporated herein in its entirety by reference

(i) Binary Regulatory Switches

In some embodiments, the ceDNA vector comprises a regulatory switch that can serve to controllably modulate expression of the transgene. For example, the expression cassette located between the ITRs of the ceDNA vector may additionally comprise a regulatory region, e.g., a promoter, cis-element, repressor, enhancer etc., that is operatively linked to the gene of interest, where the regulatory region is regulated by one or more cofactors or exogenous agents. By way of example only, regulatory regions can be modulated by small molecule switches or inducible or repressible promoters. Nonlimiting examples of inducible promoters are hormone-inducible or metal-inducible promoters. Other exemplary inducible promoters/enhancer elements include, but are not limited to, an RU486-inducible promoter, an ecdysone-inducible promoter, a rapamycin-inducible promoter, and a metallothionein promoter.

(ii) Small Molecule Regulatory Switches

A variety of art-known small-molecule based regulatory switches are known in the art and can be combined with the ceDNA vectors disclosed herein to form a regulatory-switch controlled ceDNA vector. In some embodiments, the regulatory switch can be selected from any one or a combination of: an orthogonal ligand/nuclear receptor pair, for example retinoid receptor variant/LG335 and GRQCIMFI, along with an artificial promoter controlling expression of the operatively linked transgene, such as that as disclosed in Taylor, et al. BMC Biotechnology 10 (2010): 15; engineered steroid receptors, e.g., modified progesterone receptor with a C-terminal truncation that cannot bind progesterone but binds RU486 (mifepristone) (U.S. Pat. No. 5,364,791); an ecdysone receptor from Drosophila and their ecdysteroid ligands (Saez, et al., PNAS, 97(26)(2000), 14512-14517; or a switch controlled by the antibiotic trimethoprim (TMP), as disclosed in Sando R 3^{rd}; Nat Methods. 2013, 10(11):1085-8. In some embodiments, the regulatory switch to control the transgene or expressed by the ceDNA vector is a pro-drug activation switch, such as that disclosed in U.S. Pat. Nos. 8,771,679, and 6,339,070.

(iii) “Passcode” Regulatory Switches

In some embodiments the regulatory switch can be a “passcode switch” or “passcode circuit”. Passcode switches allow fine tuning of the control of the expression of the transgene from the ceDNA vector when specific conditions occur—that is, a combination of conditions need to be present for transgene expression and/or repression to occur. For example, for expression of a transgene to occur at least conditions A and B must occur. A passcode regulatory switch can be any number of conditions, e.g., at least 2, or at least 3, or at least 4, or at least 5, or at least 6 or at least 7 or more conditions to be present for transgene expression to occur. In some embodiments, at least 2 conditions (e.g., A, B conditions) need to occur, and in some embodiments, at least 3 conditions need to occur (e.g., A, B and C, or A, B and D). By way of an example only, for gene expression from a ceDNA to occur that has a passcode “ABC” regulatory switch, conditions A, B and C must be present. Conditions A, B and C could be as follows; condition A is the presence of a condition or disease, condition B is a hormonal response, and condition C is a response to the transgene expression. For example, if the transgene edits a defective EPO gene, Condition A is the presence of Chronic Kidney Disease (CKD), Condition B occurs if the subject has hypoxic conditions in the kidney, Condition C is that Erythropoietin-producing cells (EPC) recruitment in the kidney is impaired; or alternatively, HIF-2 activation is impaired. Once the oxygen levels increase or the desired level of EPO is reached, the transgene turns off again until 3 conditions occur, turning it back on.

In some embodiments, a passcode regulatory switch or “Passcode circuit” encompassed for use in the ceDNA vector comprises hybrid transcription factors (TFs) to expand the range and complexity of environmental signals used to define biocontainment conditions. As opposed to a deadman switch which triggers cell death in the presence of a predetermined condition, the “passcode circuit” allows cell survival or transgene expression in the presence of a particular “passcode”, and can be easily reprogrammed to allow transgene expression and/or cell survival only when the predetermined environmental condition or passcode is present.

Any and all combinations of regulatory switches disclosed herein, e.g., small molecule switches, nucleic acid-based switches, small molecule-nucleic acid hybrid switches, post-transcriptional transgene regulation switches, post-translational regulation, radiation-controlled switches, hypoxia-mediated switches and other regulatory switches known by persons of ordinary skill in the art as disclosed herein can be used in a passcode regulatory switch as disclosed herein. Regulatory switches encompassed for use are also discussed in the review article Kis et al., J R Soc Interface. 12: 20141000 (2015), and summarized in Table 1 of Kis. In some embodiments, a regulatory switch for use in a passcode system can be selected from any or a combination of the switches in Table 11.

(iv). Nucleic Acid-Based Regulatory Switches to Control Transgene Expression

In some embodiments, the regulatory switch to control the transgene expressed by the ceDNA is based on a nucleic-acid based control mechanism. Exemplary nucleic acid control mechanisms are known in the art and are envisioned for use. For example, such mechanisms include riboswiches, such as those disclosed in, e.g., US2009/0305253, US2008/0269258, US2017/0204477, WO2018026762A1, U.S. Pat. No. 9,222,093 and EP application EP288071, and also disclosed in the review by Villa J K et al., Microbiol Spectr. 2018 May; 6(3). Also included are metabolite-responsive transcription biosensors, such as those disclosed in WO2018/075486 and WO2017/147585. Other art-known mechanisms envisioned for use include silencing of the transgene with an siRNA or RNAi molecule (e.g., miR, shRNA). For example, the ceDNA vector can comprise a regulatory switch that encodes a RNAi molecule that is complementary to the transgene expressed by the ceDNA vector. When such RNAi is expressed even if the transgene is expressed by the ceDNA vector, it will be silenced by the complementary RNAi molecule, and when the RNAi is not expressed when the transgene is expressed by the ceDNA vector the transgene is not silenced by the RNAi.

In some embodiments, the regulatory switch is a tissue-specific self-inactivating regulatory switch, for example as disclosed in US2002/0022018, whereby the regulatory switch deliberately switches transgene expression off at a site where transgene expression might otherwise be disadvantageous. In some embodiments, the regulatory switch is a recombinase reversible gene expression system, for example as disclosed in US2014/0127162 and U.S. Pat. No. 8,324,436.

(v). Post-Transcriptional and Post-Translational Regulatory Switches.

In some embodiments, the regulatory switch to control the transgene or gene of interest expressed by the ceDNA vector is a post-transcriptional modification system. For example, such a regulatory switch can be an aptazyme riboswitch that is sensitive to tetracycline or theophylline, as disclosed in US2018/0119156, GB201107768, WO2001/064956A3, EP Patent 2707487 and Beilstein et al., ACS Synth. Biol., 2015, 4 (5), pp 526-534; Thong et al., Elife. 2016 Nov. 2; 5. pii: e18858. In some embodiments, it is envisioned that a person of ordinary skill in the art could encode both the transgene and an inhibitory siRNA which contains a ligand sensitive (OFF-switch) aptamer, the net result being a ligand sensitive ON-switch.

(vi). Other Exemplary Regulatory Switches

Any known regulatory switch can be used in the ceDNA vector to control the gene expression of the transgene expressed by the ceDNA vector, including those triggered by environmental changes. Additional examples include, but are not limited to; the BOC method of Suzuki et al., Scientific Reports 8; 10051 (2018); genetic code expansion and a non-physiologic amino acid; radiation-controlled or ultra-sound controlled on/off switches (see, e.g., Scott S et al., Gene Ther. 2000 July; 7(13):1121-5; U.S. Pat. Nos. 5,612,318; 5,571,797; 5,770,581; 5,817,636; and WO1999/025385A1. In some embodiments, the regulatory switch is controlled by an implantable system, e.g., as disclosed in U.S. Pat. No. 7,840,263; US2007/0190028A1 where gene expression is controlled by one or more forms of energy, including electromagnetic energy, that activates promoters operatively linked to the transgene in the ceDNA vector.

In some embodiments, a regulatory switch envisioned for use in the ceDNA vector is a hypoxia-mediated or stress-activated switch, e.g., such as those disclosed in WO1999060142A2, U.S. Pat. Nos. 5,834,306; 6,218,179; 6,709,858; US2015/0322410; Greco et al., (2004) Targeted Cancer Therapies 9, 5368, as well as FROG, TOAD and NRSE elements and conditionally inducable silence elements, including hypoxia response elements (HREs), inflammatory response elements (IREs) and shear-stress activated elements (SSAEs), e.g., as disclosed in U.S. Pat. No. 9,394,526. Such an embodiment is useful for turning on expression of the transgene from the ceDNA vector after ischemia or in ischemic tissues, and/or tumors.

(iv). Kill Switches

Other embodiments of the invention relate to a ceDNA vector comprising a kill switch. A kill switch as disclosed herein enables a cell comprising the ceDNA vector to be killed or undergo programmed cell death as a means to permanently remove an introduced ceDNA vector from the subject's system. It will be appreciated by one of ordinary skill in the art that use of kill switches in the ceDNA vectors of the invention would be typically coupled with targeting of the ceDNA vector to a limited number of cells that the subject can acceptably lose or to a cell type where apoptosis is desirable (e.g., cancer cells). In all aspects, a “kill switch” as disclosed herein is designed to provide rapid and robust cell killing of the cell comprising the ceDNA vector in the absence of an input survival signal or other specified condition. Stated another way, a kill switch encoded by a ceDNA vector herein can restrict cell survival of a cell comprising a ceDNA vector to an environment defined by specific input signals. Such kill switches serve as a biological biocontainment function should it be desirable to remove the ceDNA vector from a subject or to ensure that it will not express the encoded transgene.

### VII. Detailed Method of Production of a ceDNA Vector

A. Production in General

Certain methods for the production of a ceDNA vector for gene editing comprising an asymmetrical ITR pair or symmetrical ITR pair as defined herein is described in section IV of PCT/US18/49996 filed Sep. 7, 2018, which is incorporated herein in its entirety by reference. As described herein, the ceDNA vector can be obtained, for example, by the process comprising the steps of: a) incubating a population of host cells (e.g. insect cells) harboring the polynucleotide expression construct template (e.g., a ceDNA-plasmid, a ceDNA-Bacmid, and/or a ceDNA-baculovirus), which is devoid of viral capsid coding sequences, in the presence of a Rep protein under conditions effective and for a time sufficient to induce production of the ceDNA vector within the host cells, and wherein the host cells do not comprise viral capsid coding sequences; and b) harvesting and isolating the ceDNA vector from the host cells. The presence of Rep protein induces replication of the vector polynucleotide with a modified ITR to produce the ceDNA vector in a host cell. However, no viral particles (e.g. AAV virions) are expressed. Thus, there is no size limitation such as that naturally imposed in AAV or other viral-based vectors.

The presence of the ceDNA vector isolated from the host cells can be confirmed by digesting DNA isolated from the host cell with a restriction enzyme having a single recognition site on the ceDNA vector and analyzing the digested DNA material on a non-denaturing gel to confirm the presence of characteristic bands of linear and continuous DNA as compared to linear and non-continuous DNA.

In yet another aspect, the invention provides for use of host cell lines that have stably integrated the DNA vector polynucleotide expression template (ceDNA template) into their own genome in production of the non-viral DNA vector, e.g. as described in Lee, L. et al. (2013) Plos One 8(8): e69879. Preferably, Rep is added to host cells at an MOI of about 3. When the host cell line is a mammalian cell line, e.g., HEK293 cells, the cell lines can have polynucleotide vector template stably integrated, and a second vector such as herpes virus can be used to introduce Rep protein into cells, allowing for the excision and amplification of ceDNA in the presence of Rep and helper virus.

In one embodiment, the host cells used to make the ceDNA vectors described herein are insect cells, and baculovirus is used to deliver both the polynucleotide that encodes Rep protein and the non-viral DNA vector polynucleotide expression construct template for ceDNA, e.g., as described in FIGS. 4A-4C and Example 1. In some embodiments, the host cell is engineered to express Rep protein.

The ceDNA vector is then harvested and isolated from the host cells. The time for harvesting and collecting ceDNA vectors described herein from the cells can be selected and optimized to achieve a high-yield production of the ceDNA vectors. For example, the harvest time can be selected in view of cell viability, cell morphology, cell growth, etc. In one embodiment, cells are grown under sufficient conditions and harvested a sufficient time after baculoviral infection to produce ceDNA vectors but before a majority of cells start to die because of the baculoviral toxicity. The DNA vectors can be isolated using plasmid purification kits such as Qiagen Endo-Free Plasmid kits. Other methods developed for plasmid isolation can be also adapted for DNA vectors. Generally, any nucleic acid purification methods can be adopted.

The DNA vectors can be purified by any means known to those of skill in the art for purification of DNA. In one embodiment, ceDNA vectors are purified as DNA molecules. In another embodiment, the ceDNA vectors are purified as exosomes or microparticles.

The presence of the ceDNA vector can be confirmed by digesting the vector DNA isolated from the cells with a restriction enzyme having a single recognition site on the DNA vector and analyzing both digested and undigested DNA material using gel electrophoresis to confirm the presence of characteristic bands of linear and continuous DNA as compared to linear and non-continuous DNA. FIG. 4C and FIG. 4D illustrate one embodiment for identifying the presence of the closed ended ceDNA vectors produced by the processes herein.

B. ceDNA Plasmid

A ceDNA-plasmid is a plasmid used for later production of a ceDNA vector. In some embodiments, a ceDNA-plasmid can be constructed using known techniques to provide at least the following as operatively linked components in the direction of transcription: (1) a modified 5′ ITR sequence; (2) an expression cassette containing a cis-regulatory element, for example, a promoter, inducible promoter, regulatory switch, enhancers and the like; and (3) a modified 3′ ITR sequence, where the 3′ ITR sequence is symmetric relative to the 5′ ITR sequence. In some embodiments, the expression cassette flanked by the ITRs comprises a cloning site for introducing an exogenous sequence. The expression cassette replaces the rep and cap coding regions of the AAV genomes.

In one aspect, a ceDNA vector is obtained from a plasmid, referred to herein as a “ceDNA-plasmid” encoding in this order: a first adeno-associated virus (AAV) inverted terminal repeat (ITR), an expression cassette comprising a transgene, and a mutated or modified AAV ITR, wherein said ceDNA-plasmid is devoid of AAV capsid protein coding sequences. In alternative embodiments, the ceDNA-plasmid encodes in this order: a first (or 5′) modified or mutated AAV ITR, an expression cassette comprising a transgene, and a second (or 3′) modified AAV ITR, wherein said ceDNA-plasmid is devoid of AAV capsid protein coding sequences, and wherein the 5′ and 3′ ITRs are symmetric relative to each other. In alternative embodiments, the ceDNA-plasmid encodes in this order: a first (or 5′) modified or mutated AAV ITR, an expression cassette comprising a transgene, and a second (or 3′) mutated or modified AAV ITR, wherein said ceDNA-plasmid is devoid of AAV capsid protein coding sequences, and wherein the 5′ and 3′ modified ITRs are have the same modifications (i.e., they are inverse complement or symmetric relative to each other).

In a further embodiment, the ceDNA-plasmid system is devoid of viral capsid protein coding sequences (i.e. it is devoid of AAV capsid genes but also of capsid genes of other viruses). In addition, in a particular embodiment, the ceDNA-plasmid is also devoid of AAV Rep protein coding sequences. Accordingly, in a preferred embodiment, ceDNA-plasmid is devoid of functional AAV cap and AAV rep genes GG-3′ for AAV2) plus a variable palindromic sequence allowing for hairpin formation.

A ceDNA-plasmid of the present invention can be generated using natural nucleotide sequences of the genomes of any AAV serotypes well known in the art. In one embodiment, the ceDNA-plasmid backbone is derived from the AAV1, AAV2, AAV3, AAV4, AAV5, AAV 5, AAV7, AAV8, AAV9, AAV10, AAV 11, AAV12, AAVrh8, AAVrh10, AAV-DJ, and AAV-DJ8 genome. E.g., NCBI: NC 002077; NC 001401; NC001729; NC001829; NC006152; NC 006260; NC 006261; Kotin and Smith, The Springer Index of Viruses, available at the URL maintained by Springer (at www web address: oesys.springer.de/viruses/database/mkchapter.asp?virID=42.04.)(note—references to a URL or database refer to the contents of the URL or database as of the effective filing date of this application) In a particular embodiment, the ceDNA-plasmid backbone is derived from the AAV2 genome. In another particular embodiment, the ceDNA-plasmid backbone is a synthetic backbone genetically engineered to include at its 5′ and 3′ ITRs derived from one of these AAV genomes.

A ceDNA-plasmid can optionally include a selectable or selection marker for use in the establishment of a ceDNA vector-producing cell line. In one embodiment, the selection marker can be inserted downstream (i.e., 3′) of the 3′ ITR sequence. In another embodiment, the selection marker can be inserted upstream (i.e., 5′) of the 5′ ITR sequence. Appropriate selection markers include, for example, those that confer drug resistance. Selection markers can be, for example, a blasticidin S-resistance gene, kanamycin, geneticin, and the like. In a preferred embodiment, the drug selection marker is a blasticidin S-resistance gene.

An Exemplary ceDNA (e.g., rAAVO) is produced from an rAAV plasmid. A method for the production of a rAAV vector, can comprise: (a) providing a host cell with a rAAV plasmid as described above, wherein both the host cell and the plasmid are devoid of capsid protein encoding genes, (b) culturing the host cell under conditions allowing production of an ceDNA genome, and (c) harvesting the cells and isolating the AAV genome produced from said cells.

C. Exemplary Method of Making the ceDNA Vectors from ceDNA Plasmids

Methods for making capsid-less ceDNA vectors are also provided herein, notably a method with a sufficiently high yield to provide sufficient vector for in vivo experiments.

In some embodiments, a method for the production of a ceDNA vector comprises the steps of: (1) introducing the nucleic acid construct comprising an expression cassette and two symmetric ITR sequences into a host cell (e.g., Sf9 cells), (2) optionally, establishing a clonal cell line, for example, by using a selection marker present on the plasmid, (3) introducing a Rep coding gene (either by transfection or infection with a baculovirus carrying said gene) into said insect cell, and (4) harvesting the cell and purifying the ceDNA vector. The nucleic acid construct comprising an expression cassette and two ITR sequences described above for the production of ceDNA vector can be in the form of a ceDNA plasmid, or Bacmid or Baculovirus generated with the ceDNA plasmid as described below. The nucleic acid construct can be introduced into a host cell by transfection, viral transduction, stable integration, or other methods known in the art.

D. Cell Lines:

Host cell lines used in the production of a ceDNA vector can include insect cell lines derived from Spodoptera frugiperda, such as Sf9 Sf21, or Trichoplusia ni cell, or other invertebrate, vertebrate, or other eukaryotic cell lines including mammalian cells. Other cell lines known to an ordinarily skilled artisan can also be used, such as HEK293, Huh-7, HeLa, HepG2, HeplA, 911, CHO, COS, MeWo, NIH3T3, A549, HT1 180, monocytes, and mature and immature dendritic cells. Host cell lines can be transfected for stable expression of the ceDNA-plasmid for high yield ceDNA vector production.

CeDNA-plasmids can be introduced into Sf9 cells by transient transfection using reagents (e.g., liposomal, calcium phosphate) or physical means (e.g., electroporation) known in the art. Alternatively, stable Sf9 cell lines which have stably integrated the ceDNA-plasmid into their genomes can be established. Such stable cell lines can be established by incorporating a selection marker into the ceDNA-plasmid as described above. If the ceDNA-plasmid used to transfect the cell line includes a selection marker, such as an antibiotic, cells that have been transfected with the ceDNA-plasmid and integrated the ceDNA-plasmid DNA into their genome can be selected for by addition of the antibiotic to the cell growth media. Resistant clones of the cells can then be isolated by single-cell dilution or colony transfer techniques and propagated.

E. Isolating and Purifying ceDNA Vectors:

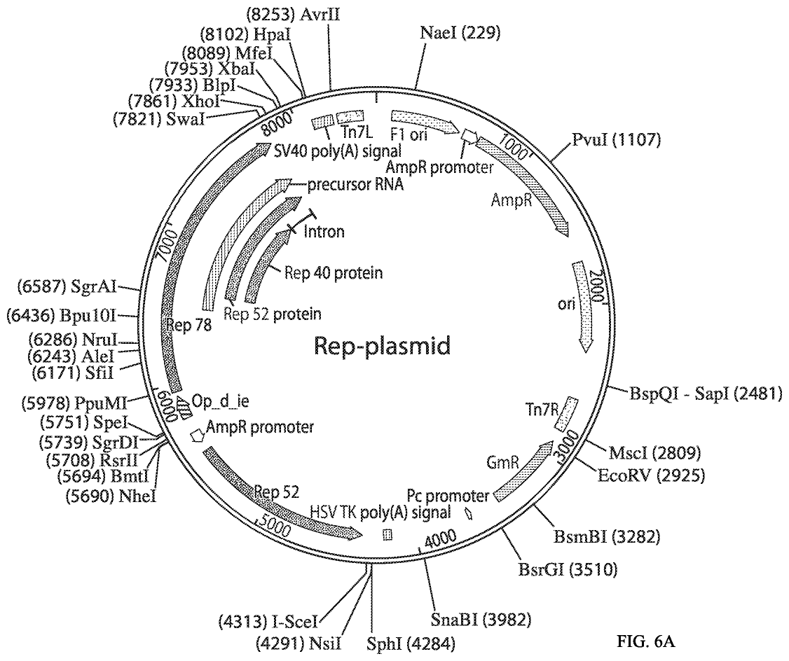

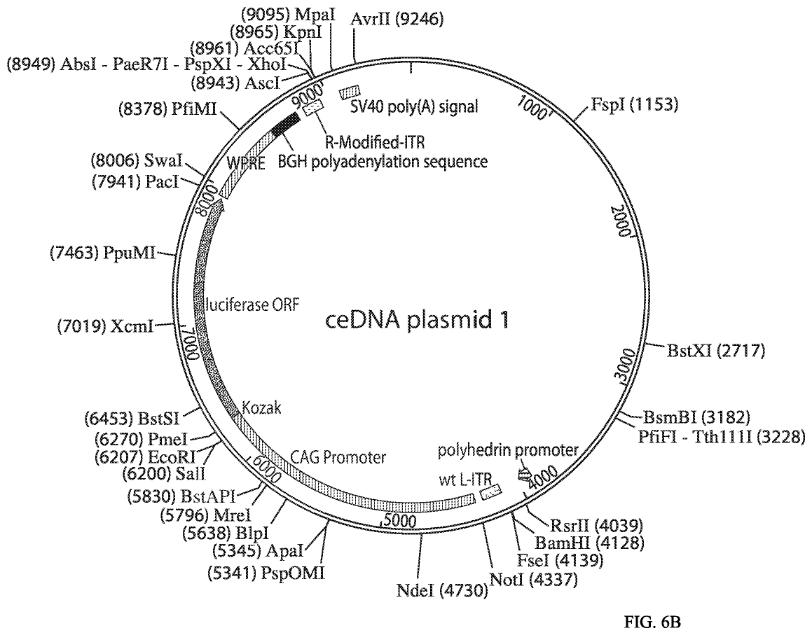

Examples of the process for obtaining and isolating ceDNA vectors for gene editing are described in FIGS. 4A-4E and the specific examples below. ceDNA-vectors disclosed herein can be obtained from a producer cell expressing AAV Rep protein(s), further transformed with a ceDNA-plasmid, ceDNA-bacmid, or ceDNA-baculovirus. Plasmids useful for the production of ceDNA vectors include plasmids shown in FIG. 6A (useful for Rep BIICs production), FIG. 6B (plasmid used to obtain a ceDNA vector).

In one aspect, a polynucleotide encodes the AAV Rep protein (Rep 78 or 68) delivered to a producer cell in a plasmid (Rep-plasmid), a bacmid (Rep-bacmid), or a baculovirus (Rep-baculovirus). The Rep-plasmid, Rep-bacmid, and Rep-baculovirus can be generated by methods described above.

Methods to produce a ceDNA-vector, which is an exemplary ceDNA vector, are described herein. Expression constructs used for generating a ceDNA vectors of the present invention can be a plasmid (e.g., ceDNA-plasmids), a Bacmid (e.g., ceDNA-bacmid), and/or a baculovirus (e.g., ceDNA-baculovirus). By way of an example only, a ceDNA-vector can be generated from the cells co-infected with ceDNA-baculovirus and Rep-baculovirus. Rep proteins produced from the Rep-baculovirus can replicate the ceDNA-baculovirus to generate ceDNA-vectors. Alternatively, ceDNA vectors can be generated from the cells stably transfected with a construct comprising a sequence encoding the AAV Rep protein (Rep78/52) delivered in Rep-plasmids, Rep-bacmids, or Rep-baculovirus. CeDNA-Baculovirus can be transiently transfected to the cells, be replicated by Rep protein and produce ceDNA vectors.

The bacmid (e.g., ceDNA-bacmid) can be transfected into a permissive insect cells such as Sf9, Sf21, Tni (Trichoplusia ni) cell, High Five cell, and generate ceDNA-baculovirus, which is a recombinant baculovirus including the sequences comprising the symmetric ITRs and the expression cassette. ceDNA-baculovirus can be again infected into the insect cells to obtain a next generation of the recombinant baculovirus. Optionally, the step can be repeated once or multiple times to produce the recombinant baculovirus in a larger quantity.

The time for harvesting and collecting ceDNA vectors described herein from the cells can be selected and optimized to achieve a high-yield production of the ceDNA vectors. For example, the harvest time can be selected in view of cell viability, cell morphology, cell growth, etc. Usually, cells can be harvested after sufficient time after baculoviral infection to produce ceDNA vectors (e.g., ceDNA vectors) but before majority of cells start to die because of the viral toxicity. The ceDNA-vectors can be isolated from the Sf9 cells using plasmid purification kits such as Qiagen ENDO-FREE PLASMID® kits. Other methods developed for plasmid isolation can be also adapted for ceDNA vectors. Generally, any art-known nucleic acid purification methods can be adopted, as well as commercially available DNA extraction kits.

Alternatively, purification can be implemented by subjecting a cell pellet to an alkaline lysis process, centrifuging the resulting lysate and performing chromatographic separation. As one nonlimiting example, the process can be performed by loading the supernatant on an ion exchange column (e.g. SARTOBIND Q®) which retains nucleic acids, and then eluting (e.g. with a 1.2 M NaCl solution) and performing a further chromatographic purification on a gel filtration column (e.g. 6 fast flow GE). The capsid-free AAV vector is then recovered by, e.g., precipitation.

In some embodiments, ceDNA vectors can also be purified in the form of exosomes, or microparticles. It is known in the art that many cell types release not only soluble proteins, but also complex protein/nucleic acid cargoes via membrane microvesicle shedding (Cocucci et al, 2009; EP 10306226.1) Such vesicles include microvesicles (also referred to as microparticles) and exosomes (also referred to as nanovesicles), both of which comprise proteins and RNA as cargo. Microvesicles are generated from the direct budding of the plasma membrane, and exosomes are released into the extracellular environment upon fusion of multivesicular endosomes with the plasma membrane. Thus, ceDNA vector-containing microvesicles and/or exosomes can be isolated from cells that have been transduced with the ceDNA-plasmid or a bacmid or baculovirus generated with the ceDNA-plasmid.

Microvesicles can be isolated by subjecting culture medium to filtration or ultracentrifugation at 20,000×g, and exosomes at 100,000×g. The optimal duration of ultracentrifugation can be experimentally-determined and will depend on the particular cell type from which the vesicles are isolated. Preferably, the culture medium is first cleared by low-speed centrifugation (e.g., at 2000×g for 5-20 minutes) and subjected to spin concentration using, e.g., an AMICON® spin column (Millipore, Watford, UK). Microvesicles and exosomes can be further purified via FACS or MACS by using specific antibodies that recognize specific surface antigens present on the microvesicles and exosomes. Other microvesicle and exosome purification methods include, but are not limited to, immunoprecipitation, affinity chromatography, filtration, and magnetic beads coated with specific antibodies or aptamers. Upon purification, vesicles are washed with, e.g., phosphate-buffered saline. One advantage of using microvesicles or exosome to deliver ceDNA-containing vesicles is that these vesicles can be targeted to various cell types by including on their membranes proteins recognized by specific receptors on the respective cell types. (See also EP 10306226)

Another aspect of the invention herein relates to methods of purifying ceDNA vectors from host cell lines that have stably integrated a ceDNA construct into their own genome. In one embodiment, ceDNA vectors are purified as DNA molecules. In another embodiment, the ceDNA vectors are purified as exosomes or microparticles.

FIG. 5 of PCT/US18/49996 shows a gel confirming the production of ceDNA from multiple ceDNA-plasmid constructs using the method described in the Examples. The ceDNA is confirmed by a characteristic band pattern in the gel, as discussed with respect to FIG. 4D in the Examples.

### VIII. Pharmaceutical Compositions

In another aspect, pharmaceutical compositions are provided. The pharmaceutical composition comprises a ceDNA vector for gene editing as disclosed herein and a pharmaceutically acceptable carrier or diluent.

The gene editing DNA-vectors disclosed herein can be incorporated into pharmaceutical compositions suitable for administration to a subject for in vivo delivery to cells, tissues, or organs of the subject. Typically, the pharmaceutical composition comprises a ceDNA-vector as disclosed herein and a pharmaceutically acceptable carrier. For example, the ceDNA vectors described herein can be incorporated into a pharmaceutical composition suitable for a desired route of therapeutic administration (e.g., parenteral administration). Passive tissue transduction via high pressure intravenous or intra-arterial infusion, as well as intracellular injection, such as intranuclear microinjection or intracytoplasmic injection, are also contemplated. Pharmaceutical compositions for therapeutic purposes can be formulated as a solution, microemulsion, dispersion, liposomes, or other ordered structure suitable to high ceDNA vector concentration. Sterile injectable solutions can be prepared by incorporating the ceDNA vector compound in the required amount in an appropriate buffer with one or a combination of ingredients enumerated above, as required, followed by filtered sterilization including a ceDNA vector can be formulated to deliver a transgene in the nucleic acid to the cells of a recipient, resulting in the therapeutic expression of the transgene or donor sequence therein. The composition can also include a pharmaceutically acceptable carrier.

Pharmaceutically active compositions comprising a ceDNA vector can be formulated to deliver a transgene or donor sequence for various purposes to the cell, e.g., cells of a subject.

Pharmaceutical compositions for therapeutic purposes typically must be sterile and stable under the conditions of manufacture and storage. The composition can be formulated as a solution, microemulsion, dispersion, liposomes, or other ordered structure suitable to high ceDNA vector concentration. Sterile injectable solutions can be prepared by incorporating the ceDNA vector compound in the required amount in an appropriate buffer with one or a combination of ingredients enumerated above, as required, followed by filtered sterilization.

A ceDNA vector as disclosed herein can be incorporated into a pharmaceutical composition suitable for topical, systemic, intra-amniotic, intrathecal, intracranial, intra-arterial, intravenous, intralymphatic, intraperitoneal, subcutaneous, tracheal, intra-tissue (e.g., intramuscular, intracardiac, intrahepatic, intrarenal, intracerebral), intrathecal, intravesical, conjunctival (e.g., extra-orbital, intraorbital, retroorbital, intraretinal, subretinal, choroidal, sub-choroidal, intrastromal, intracameral and intravitreal), intracochlear, and mucosal (e.g., oral, rectal, nasal) administration. Passive tissue transduction via high pressure intravenous or intraarterial infusion, as well as intracellular injection, such as intranuclear microinjection or intracytoplasmic injection, are also contemplated.

Pharmaceutical compositions for therapeutic purposes typically must be sterile and stable under the conditions of manufacture and storage. The composition can be formulated as a solution, microemulsion, dispersion, liposomes, or other ordered structure suitable to high ceDNA vector concentration. Sterile injectable solutions can be prepared by incorporating the ceDNA vector compound in the required amount in an appropriate buffer with one or a combination of ingredients enumerated above, as required, followed by filtered sterilization.

In some aspects, the methods provided herein comprise delivering one or more ceDNA vectors for gene editing as disclosed herein to a host cell. Also provided herein are cells produced by such methods, and organisms (such as animals, plants, or fungi) comprising or produced from such cells. Methods of delivery of nucleic acids can include lipofection, nucleofection, microinjection, biolistics, liposomes, immunoliposomes, polycation or lipid:nucleic acid conjugates, naked DNA, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Delivery can be to cells (e.g., in vitro or ex vivo administration) or target tissues (e.g., in vivo administration).

Various techniques and methods are known in the art for delivering nucleic acids to cells. For example, nucleic acids, such as ceDNA can be formulated into lipid nanoparticles (LNPs), lipidoids, liposomes, lipid nanoparticles, lipoplexes, or core-shell nanoparticles. Typically, LNPs are composed of nucleic acid (e.g., ceDNA) molecules, one or more ionizable or cationic lipids (or salts thereof), one or more non-ionic or neutral lipids (e.g., a phospholipid), a molecule that prevents aggregation (e.g., PEG or a PEG-lipid conjugate), and optionally a sterol (e.g., cholesterol).

Another method for delivering nucleic acids, such as ceDNA to a cell is by conjugating the nucleic acid with a ligand that is internalized by the cell. For example, the ligand can bind a receptor on the cell surface and internalized via endocytosis. The ligand can be covalently linked to a nucleotide in the nucleic acid. Exemplary conjugates for delivering nucleic acids into a cell are described, example, in WO2015/006740, WO2014/025805, WO2012/037254, WO2009/082606, WO2009/073809, WO2009/018332, WO2006/112872, WO2004/090108, WO2004/091515 and WO2017/177326.

Nucleic acids, such as ceDNA, can also be delivered to a cell by transfection. Useful transfection methods include, but are not limited to, lipid-mediated transfection, cationic polymer-mediated transfection, or calcium phosphate precipitation. Transfection reagents are well known in the art and include, but are not limited to, TurboFect Transfection Reagent (Thermo Fisher Scientific), Pro-Ject Reagent (Thermo Fisher Scientific), TRANSPASS™ P Protein Transfection Reagent (New England Biolabs), CHARIOT™ Protein Delivery Reagent (Active Motif), PROTEOJUICE™ Protein Transfection Reagent (EMD Millipore), 293fectin, LIPOFECTAMINE™ 2000, LIPOFECTAMINE™ 3000 (Thermo Fisher Scientific), LIPOFECTAMINE™ (Thermo Fisher Scientific), LIPOFECTIN™ (Thermo Fisher Scientific), DMRIE-C, CELLFECTIN™ (Thermo Fisher Scientific), OLIGOFECTAMINE™ (Thermo Fisher Scientific), LIPOFECTACE™, FUGENE™ (Roche, Basel, Switzerland), FUGENE™ HD (Roche), TRANSFECTAM™ (Transfectam, Promega, Madison, Wis.), TFX-10™ (Promega), TFX-20™ (Promega), TFX-50™ (Promega), TRANSFECTIN™ (BioRad, Hercules, Calif.), SILENTFECT™ (Bio-Rad), Effectene™ (Qiagen, Valencia, Calif.), DC-chol (Avanti Polar Lipids), GENEPORTER™ (Gene Therapy Systems, San Diego, Calif.), DHARMAFECT 1™ (Dharmacon, Lafayette, Colo.), DHARMAFECT 2™ (Dharmacon), DHARMAFECT 3™ (Dharmacon), DHARMAFECT 4™ (Dharmacon), ESCORT™ III (Sigma, St. Louis, Mo.), and ESCORT™ IV (Sigma Chemical Co.). Nucleic acids, such as ceDNA, can also be delivered to a cell via microfluidics methods known to those of skill in the art.

Methods of non-viral delivery of nucleic acids in vivo or ex vivo include electroporation, lipofection (see, U.S. Pat. Nos. 5,049,386; 4,946,787 and commercially available reagents such as Transfectam™ and Lipofectin™), microinjection, biolistics, virosomes, liposomes (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787), immunoliposomes, polycation or lipid:nucleic acid conjugates, naked DNA, and agent-enhanced uptake of DNA. Sonoporation using, e.g., the Sonitron 2000 system (Rich-Mar) can also be used for delivery of nucleic acids.

ceDNA vectors as described herein can also be administered directly to an organism for transduction of cells in vivo. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Methods for introduction of a nucleic acid vector ceDNA vector as disclosed herein can be delivered into hematopoietic stem cells, for example, by the methods as decribed, for example, in U.S. Pat. No. 5,928,638.

The ceDNA vectors in accordance with the present invention can be added to liposomes for delivery to a cell or target organ in a subject. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids.

In some aspects, the disclosure provides for a liposome formulation that includes one or more compounds with a polyethylene glycol (PEG) functional group (so-called “PEG-ylated compounds”) which can reduce the immunogenicity/antigenicity of, provide hydrophilicity and hydrophobicity to the compound(s) and reduce dosage frequency. Or the liposome formulation simply includes polyethylene glycol (PEG) polymer as an additional component. In such aspects, the molecular weight of the PEG or PEG functional group can be from 62 Da to about 5,000 Da.

In some aspects, the disclosure provides for a liposome formulation that will deliver an API with extended release or controlled release profile over a period of hours to weeks. In some related aspects, the liposome formulation may comprise aqueous chambers that are bound by lipid bilayers. In other related aspects, the liposome formulation encapsulates an API with components that undergo a physical transition at elevated temperature which releases the API over a period of hours to weeks.

In some aspects, the liposome formulation comprises sphingomyelin and one or more lipids disclosed herein. In some aspects, the liposome formulation comprises optisomes.

In some aspects, the disclosure provides for a liposome formulation that includes one or more lipids selected from: N-(carbonyl-methoxypolyethylene glycol 2000)-1,2-distearoyl-sn-glycero-3-phosphoethanolamine sodium salt, (distearoyl-sn-glycero-phosphoethanolamine), MPEG (methoxy polyethylene glycol)-conjugated lipid, HSPC (hydrogenated soy phosphatidylcholine); PEG (polyethylene glycol); DSPE (distearoyl-sn-glycero-phosphoethanolamine); DSPC (distearoylphosphatidylcholine); DOPC (dioleoylphosphatidylcholine); DPPG (dipalmitoylphosphatidylglycerol); EPC (egg phosphatidylcholine); DOPS (dioleoylphosphatidylserine); POPC (palmitoyloleoylphosphatidylcholine); SM (sphingomyelin); MPEG (methoxy polyethylene glycol); DMPC (dimyristoyl phosphatidylcholine); DMPG (dimyristoyl phosphatidylglycerol); DSPG (distearoylphosphatidylglycerol); DEPC (dierucoylphosphatidylcholine); DOPE (dioleoly-sn-glycero-phophoethanolamine) cholesteryl sulphate (CS), dipalmitoylphosphatidylglycerol (DPPG), DOPC (dioleoly-sn-glycero-phosphatidylcholine) or any combination thereof.

In some aspects, the disclosure provides for a liposome formulation comprising phospholipid, cholesterol and a PEG-ylated lipid in a molar ratio of 56:38:5. In some aspects, the liposome formulation's overall lipid content is from 2-16 mg/mL. In some aspects, the disclosure provides for a liposome formulation comprising a lipid containing a phosphatidylcholine functional group, a lipid containing an ethanolamine functional group and a PEG-ylated lipid. In some aspects, the disclosure provides for a liposome formulation comprising a lipid containing a phosphatidylcholine functional group, a lipid containing an ethanolamine functional group and a PEG-ylated lipid in a molar ratio of 3:0.015:2 respectively. In some aspects, the disclosure provides for a liposome formulation comprising a lipid containing a phosphatidylcholine functional group, cholesterol and a PEG-ylated lipid. In some aspects, the disclosure provides for a liposome formulation comprising a lipid containing a phosphatidylcholine functional group and cholesterol. In some aspects, the PEG-ylated lipid is PEG-2000-DSPE. In some aspects, the disclosure provides for a liposome formulation comprising DPPG, soy PC, MPEG-DSPE lipid conjugate and cholesterol.

In some aspects, the disclosure provides for a liposome formulation comprising one or more lipids containing a phosphatidylcholine functional group and one or more lipids containing an ethanolamine functional group. In some aspects, the disclosure provides for a liposome formulation comprising one or more: lipids containing a phosphatidylcholine functional group, lipids containing an ethanolamine functional group, and sterols, e.g. cholesterol. In some aspects, the liposome formulation comprises DOPC/DEPC; and DOPE.

In some aspects, the disclosure provides for a liposome formulation further comprising one or more pharmaceutical excipients, e.g. sucrose and/or glycine.

In some aspects, the disclosure provides for a liposome formulation that is wither unilamellar or multilamellar in structure. In some aspects, the disclosure provides for a liposome formulation that comprises multi-vesicular particles and/or foam-based particles. In some aspects, the disclosure provides for a liposome formulation that are larger in relative size to common nanoparticles and about 150 to 250 nm in size. In some aspects, the liposome formulation is a lyophilized powder.

In some aspects, the disclosure provides for a liposome formulation that is made and loaded with ceDNA vectors disclosed or described herein, by adding a weak base to a mixture having the isolated ceDNA outside the liposome. This addition increases the pH outside the liposomes to approximately 7.3 and drives the API into the liposome. In some aspects, the disclosure provides for a liposome formulation having a pH that is acidic on the inside of the liposome. In such cases the inside of the liposome can be at pH 4-6.9, and more preferably pH 6.5. In other aspects, the disclosure provides for a liposome formulation made by using intra-liposomal drug stabilization technology. In such cases, polymeric or non-polymeric highly charged anions and intra-liposomal trapping agents are utilized, e.g. polyphosphate or sucrose octasulfate.

In other aspects, the disclosure provides for a liposome formulation comprising phospholipids, lecithin, phosphatidylcholine and phosphatidylethanolamine.

Delivery reagents such as liposomes, nanocapsules, microparticles, microspheres, lipid particles, vesicles, and the like, can be used for the introduction of the compositions of the present disclosure into suitable host cells. In particular, the nucleic acids can be formulated for delivery either encapsulated in a lipid particle, a liposome, a vesicle, a nanosphere, a nanoparticle, a gold particle, or the like. Such formulations can be preferred for the introduction of pharmaceutically acceptable formulations of the nucleic acids disclosed herein.

Various delivery methods known in the art or modification thereof can be used to deliver ceDNA vectors in vitro or in vivo. For example, in some embodiments, ceDNA vectors are delivered by making transient penetration in cell membrane by mechanical, electrical, ultrasonic, hydrodynamic, or laser-based energy so that DNA entrance into the targeted cells is facilitated. For example, a ceDNA vector can be delivered by transiently disrupting cell membrane by squeezing the cell through a size-restricted channel or by other means known in the art. In some cases, a ceDNA vector alone is directly injected as naked DNA into skin, thymus, cardiac muscle, skeletal muscle, or liver cells.

In some cases, a ceDNA vector is delivered by gene gun. Gold or tungsten spherical particles (1-3 μm diameter) coated with capsid-free AAV vectors can be accelerated to high speed by pressurized gas to penetrate into target tissue cells.

Compositions comprising a ceDNA vector and a pharmaceutically acceptable carrier are specifically contemplated herein. In some embodiments, the ceDNA vector is formulated with a lipid delivery system, for example, liposomes as described herein. In some embodiments, such compositions are administered by any route desired by a skilled practitioner. The compositions may be administered to a subject by different routes including orally, parenterally, sublingually, transdermally, rectally, transmucosally, topically, via inhalation, via buccal administration, intrapleurally, intravenous, intra-arterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian may readily determine the dosing regimen and route of administration that is most appropriate for a particular animal. The compositions may be administered by traditional syringes, needleless injection devices, “microprojectile bombardment gone guns”, or other physical methods such as electroporation (“EP”), “hydrodynamic method”, or ultrasound.

The composition can be delivered to a subject by several technologies including DNA injection (also referred to as DNA vaccination) with and without in vivo electroporation, liposome mediated, or nanoparticle facilitated, as described herein.

In some embodiments, electroporation is used to deliver ceDNA vectors. Electroporation causes temporary destabilization of the cell membrane target cell tissue by insertion of a pair of electrodes into the tissue so that DNA molecules in the surrounding media of the destabilized membrane would be able to penetrate into cytoplasm and nucleoplasm of the cell. Electroporation has been used in vivo for many types of tissues, such as skin, lung, and muscle.

In some cases, a ceDNA vector is delivered by hydrodynamic injection, which is a simple and highly efficient method for direct intracellular delivery of any water-soluble compounds and particles into internal organs and skeletal muscle in an entire limb.

In some cases, ceDNA vectors are delivered by ultrasound by making nanoscopic pores in membrane to facilitate intracellular delivery of DNA particles into cells of internal organs or tumors, so the size and concentration of plasmid DNA have great role in efficiency of the system. In some cases, ceDNA vectors are delivered by magnetofection by using magnetic fields to concentrate particles containing nucleic acid into the target cells.

In some cases, chemical delivery systems can be used, for example, by using nanomeric complexes, which include compaction of negatively charged nucleic acid by polycationic nanomeric particles, belonging to cationic liposome/micelle or cationic polymers. Cationic lipids used for the delivery method includes, but not limited to monovalent cationic lipids, polyvalent cationic lipids, guanidine containing compounds, cholesterol derivative compounds, cationic polymers, (e.g., poly(ethylenimine), poly-L-lysine, protamine, other cationic polymers), and lipid-polymer hybrid.

A. Exosomes:

In some embodiments, a ceDNA vector as disclosed herein is delivered by being packaged in an exosome. Exosomes are small membrane vesicles of endocytic origin that are released into the extracellular environment following fusion of multivesicular bodies with the plasma membrane. Their surface consists of a lipid bilayer from the donor cell's cell membrane, they contain cytosol from the cell that produced the exosome, and exhibit membrane proteins from the parental cell on the surface. Exosomes are produced by various cell types including epithelial cells, B and T lymphocytes, mast cells (MC) as well as dendritic cells (DC). Some embodiments, exosomes with a diameter between 10 nm and 1 μm, between 20 nm and 500 nm, between 30 nm and 250 nm, between 50 nm and 100 nm are envisioned for use. Exosomes can be isolated for a delivery to target cells using either their donor cells or by introducing specific nucleic acids into them. Various approaches known in the art can be used to produce exosomes containing capsid-free AAV vectors of the present invention.

B. Microparticle/Nanoparticles:

In some embodiments, a ceDNA vector as disclosed herein is delivered by a lipid nanoparticle. Generally, lipid nanoparticles comprise an ionizable amino lipid (e.g., heptatriaconta-6,9,28,31-tetraen-19-yl 4-(dimethylamino)butanoate, DLin-MC3-DMA, a phosphatidylcholine (1,2-distearoyl-sn-glycero-3-phosphocholine, DSPC), cholesterol and a coat lipid (polyethylene glycol-dimyristolglycerol, PEG-DMG), for example as disclosed by Tam et al. (2013). Advances in Lipid Nanoparticles for siRNA delivery. Pharmaceuticals 5(3): 498-507.

In some embodiments, a lipid nanoparticle has a mean diameter between about 10 and about 1000 nm. In some embodiments, a lipid nanoparticle has a diameter that is less than 300 nm. In some embodiments, a lipid nanoparticle has a diameter between about 10 and about 300 nm. In some embodiments, a lipid nanoparticle has a diameter that is less than 200 nm. In some embodiments, a lipid nanoparticle has a diameter between about 25 and about 200 nm. In some embodiments, a lipid nanoparticle preparation (e.g., composition comprising a plurality of lipid nanoparticles) has a size distribution in which the mean size (e.g., diameter) is about 70 nm to about 200 nm, and more typically the mean size is about 100 nm or less.

Various lipid nanoparticles known in the art can be used to deliver ceDNA vector disclosed herein. For example, various delivery methods using lipid nanoparticles are described in U.S. Pat. Nos. 9,404,127, 9,006,417 and 9,518,272.

In some embodiments, a ceDNA vector disclosed herein is delivered by a gold nanoparticle. Generally, a nucleic acid can be covalently bound to a gold nanoparticle or non-covalently bound to a gold nanoparticle (e.g., bound by a charge-charge interaction), for example as described by Ding et al. (2014). Gold Nanoparticles for Nucleic Acid Delivery. Mol. Ther. 22(6); 1075-1083. In some embodiments, gold nanoparticle-nucleic acid conjugates are produced using methods described, for example, in U.S. Pat. No. 6,812,334.

C. Conjugates

In some embodiments, a ceDNA vector as disclosed herein is conjugated (e.g., covalently bound to an agent that increases cellular uptake. An “agent that increases cellular uptake” is a molecule that facilitates transport of a nucleic acid across a lipid membrane. For example, a nucleic acid can be conjugated to a lipophilic compound (e.g., cholesterol, tocopherol, etc.), a cell penetrating peptide (CPP) (e.g., penetratin, TAT, Syn1B, etc.), and polyamines (e.g., spermine). Further examples of agents that increase cellular uptake are disclosed, for example, in Winkler (2013). Oligonucleotide conjugates for therapeutic applications. Ther. Deliv. 4(7); 791-809.

In some embodiments, a ceDNA vector as disclosed herein is conjugated to a polymer (e.g., a polymeric molecule) or a folate molecule (e.g., folic acid molecule). Generally, delivery of nucleic acids conjugated to polymers is known in the art, for example as described in WO2000/34343 and WO2008/022309. In some embodiments, a ceDNA vector as disclosed herein is conjugated to a poly(amide) polymer, for example as described by U.S. Pat. No. 8,987,377. In some embodiments, a nucleic acid described by the disclosure is conjugated to a folic acid molecule as described in U.S. Pat. No. 8,507,455.

In some embodiments, a ceDNA vector as disclosed herein is conjugated to a carbohydrate, for example as described in U.S. Pat. No. 8,450,467.

D. Nanocapsule

Alternatively, nanocapsule formulations of a ceDNA vector as disclosed herein can be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

E. Liposomes

The ceDNA vectors in accordance with the present invention can be added to liposomes for delivery to a cell or target organ in a subject. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids.

The formation and use of liposomes is generally known to those of skill in the art. Liposomes have been developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 ANG, containing an aqueous solution in the core.

In some embodiments, a liposome comprises cationic lipids. The term “cationic lipid” includes lipids and synthetic lipids having both polar and non-polar domains and which are capable of being positively charged at or around physiological pH and which bind to polyanions, such as nucleic acids, and facilitate the delivery of nucleic acids into cells. In some embodiments, cationic lipids include saturated and unsaturated alkyl and alicyclic ethers and esters of amines, amides, or derivatives thereof. In some embodiments, cationic lipids comprise straight-chain, branched alkyl, alkenyl groups, or any combination of the foregoing. In some embodiments, cationic lipids contain from 1 to about 25 carbon atoms (e.g., 1, 2, 3, 4, 5, 6, 7, 8, 9, 10, 11, 12, 13, 14, 15, 16, 17, 18, 19, 20, 21, 22, 23, 24, or 25 carbon atoms. In some embodiments, cationic lipids contain more than 25 carbon atoms. In some embodiments, straight chain or branched alkyl or alkene groups have six or more carbon atoms. A cationic lipid can also comprise, in some embodiments, one or more alicyclic groups. Non-limiting examples of alicyclic groups include cholesterol and other steroid groups. In some embodiments, cationic lipids are prepared with a one or more counterions. Examples of counterions (anions) include but are not limited to Cl⁻, Br⁻, I⁻, F⁻, acetate, trifluoroacetate, sulfate, nitrite, and nitrate.

In some aspects, the disclosure provides for a liposome formulation that includes one or more compounds with a polyethylene glycol (PEG) functional group (so-called “PEG-ylated compounds”) which can reduce the immunogenicity/antigenicity of, provide hydrophilicity and hydrophobicity to the compound(s) and reduce dosage frequency. Or the liposome formulation simply includes polyethylene glycol (PEG) polymer as an additional component. In such aspects, the molecular weight of the PEG or PEG functional group can be from 62 Da to about 5,000 Da.

In some aspects, the disclosure provides for a liposome formulation that will deliver an API with extended release or controlled release profile over a period of hours to weeks. In some related aspects, the liposome formulation may comprise aqueous chambers that are bound by lipid bilayers. In other related aspects, the liposome formulation encapsulates an API with components that undergo a physical transition at elevated temperature which releases the API over a period of hours to weeks.

In some aspects, the liposome formulation comprises sphingomyelin and one or more lipids disclosed herein. In some aspects, the liposome formulation comprises optisomes.

In some aspects, the disclosure provides for a liposome formulation that includes one or more lipids selected from: N-(carbonyl-methoxypolyethylene glycol 2000)-1,2-distearoyl-sn-glycero-3-phosphoethanolamine sodium salt, (distearoyl-sn-glycero-phosphoethanolamine), MPEG (methoxy polyethylene glycol)-conjugated lipid, HSPC (hydrogenated soy phosphatidylcholine); PEG (polyethylene glycol); DSPE (distearoyl-sn-glycero-phosphoethanolamine); DSPC (distearoylphosphatidylcholine); DOPC (dioleoylphosphatidylcholine); DPPG (dipalmitoylphosphatidylglycerol); EPC (egg phosphatidylcholine); DOPS (dioleoylphosphatidylserine); POPC (palmitoyloleoylphosphatidylcholine); SM (sphingomyelin); MPEG (methoxy polyethylene glycol); DMPC (dimyristoyl phosphatidylcholine); DMPG (dimyristoyl phosphatidylglycerol); DSPG (distearoylphosphatidylglycerol); DEPC (dierucoylphosphatidylcholine); DOPE (dioleoly-sn-glycero-phophoethanolamine) cholesteryl sulphate (CS), dipalmitoylphosphatidylglycerol (DPPG), DOPC (dioleoly-sn-glycero-phosphatidylcholine) or any combination thereof.

In some aspects, the disclosure provides for a liposome formulation comprising phospholipid, cholesterol and a PEG-ylated lipid in a molar ratio of 56:38:5. In some aspects, the liposome formulation's overall lipid content is from 2-16 mg/mL. In some aspects, the disclosure provides for a liposome formulation comprising a lipid containing a phosphatidylcholine functional group, a lipid containing an ethanolamine functional group and a PEG-ylated lipid. In some aspects, the disclosure provides for a liposome formulation comprising a lipid containing a phosphatidylcholine functional group, a lipid containing an ethanolamine functional group and a PEG-ylated lipid in a molar ratio of 3:0.015:2 respectively. In some aspects, the disclosure provides for a liposome formulation comprising a lipid containing a phosphatidylcholine functional group, cholesterol and a PEG-ylated lipid. In some aspects, the disclosure provides for a liposome formulation comprising a lipid containing a phosphatidylcholine functional group and cholesterol. In some aspects, the PEG-ylated lipid is PEG-2000-DSPE. In some aspects, the disclosure provides for a liposome formulation comprising DPPG, soy PC, MPEG-DSPE lipid conjugate and cholesterol.

In some aspects, the disclosure provides for a liposome formulation comprising one or more lipids containing a phosphatidylcholine functional group and one or more lipids containing an ethanolamine functional group. In some aspects, the disclosure provides for a liposome formulation comprising one or more: lipids containing a phosphatidylcholine functional group, lipids containing an ethanolamine functional group, and sterols, e.g. cholesterol. In some aspects, the liposome formulation comprises DOPC/DEPC; and DOPE.

In some aspects, the disclosure provides for a liposome formulation further comprising one or more pharmaceutical excipients, e.g. sucrose and/or glycine.

In some aspects, the disclosure provides for a liposome formulation that is wither unilamellar or multilamellar in structure. In some aspects, the disclosure provides for a liposome formulation that comprises multi-vesicular particles and/or foam-based particles. In some aspects, the disclosure provides for a liposome formulation that are larger in relative size to common nanoparticles and about 150 to 250 nm in size. In some aspects, the liposome formulation is a lyophilized powder.

In some aspects, the disclosure provides for a liposome formulation that is made and loaded with ceDNA vectors disclosed or described herein, by adding a weak base to a mixture having the isolated ceDNA outside the liposome. This addition increases the pH outside the liposomes to approximately 7.3 and drives the API into the liposome. In some aspects, the disclosure provides for a liposome formulation having a pH that is acidic on the inside of the liposome. In such cases the inside of the liposome can be at pH 4-6.9, and more preferably pH 6.5. In other aspects, the disclosure provides for a liposome formulation made by using intra-liposomal drug stabilization technology. In such cases, polymeric or non-polymeric highly charged anions and intra-liposomal trapping agents are utilized, e.g. polyphosphate or sucrose octasulfate.

In other aspects, the disclosure provides for a liposome formulation comprising phospholipids, lecithin, phosphatidylcholine and phosphatidylethanolamine.

Non-limiting examples of cationic lipids include polyethylenimine, polyamidoamine (PAMAM) starburst dendrimers, Lipofectin (a combination of DOTMA and DOPE), Lipofectase, LIPOFECTAMINE™ (e.g., LIPOFECTAMINE™ 2000), DOPE, Cytofectin (Gilead Sciences, Foster City, Calif.), and Eufectins (JBL, San Luis Obispo, Calif.). Exemplary cationic liposomes can be made from N-[1-(2,3-dioleoloxy)-propyl]-N,N,N-trimethylammonium chloride (DOTMA), N-[1-(2,3-dioleoloxy)-propyl]-N,N,N-trimethylammonium methylsulfate (DOTAP), 3β-[N—(N′,N′-dimethylaminoethane)carbamoyl]cholesterol (DC-Chol), 2,3,-dioleyloxy-N-[2(sperminecarboxamido)ethyl]-N,N-dimethyl-1-propanaminium trifluoroacetate (DOSPA), 1,2-dimyristyloxypropyl-3-dimethyl-hydroxyethyl ammonium bromide; and dimethyldioctadecylammonium bromide (DDAB). Nucleic acids (e.g., CELiD) can also be complexed with, e.g., poly (L-lysine) or avidin and lipids can, or can not, be included in this mixture, e.g., steryl-poly (L-lysine).

In some embodiments, a ceDNA vector as disclosed herein is delivered using a cationic lipid described in U.S. Pat. No. 8,158,601, or a polyamine compound or lipid as described in U.S. Pat. No. 8,034,376.

F. Exemplary Liposome and Lipid Nanoparticle (LNP) Compositions

The ceDNA vectors in accordance with the present invention can be added to liposomes for delivery to a cell in need of gene editing, e.g., in need of a donor sequence. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids.

In some aspects, the disclosure provides for a liposome formulation that includes one or more compounds with a polyethylene glycol (PEG) functional group (so-called “PEG-ylated compounds”) which can reduce the immunogenicity/antigenicity of, provide hydrophilicity and hydrophobicity to the compound(s) and reduce dosage frequency. Or the liposome formulation simply includes polyethylene glycol (PEG) polymer as an additional component. In such aspects, the molecular weight of the PEG or PEG functional group can be from 62 Da to about 5,000 Da.

In some aspects, the disclosure provides for a liposome formulation that will deliver an API with extended release or controlled release profile over a period of hours to weeks. In some related aspects, the liposome formulation may comprise aqueous chambers that are bound by lipid bilayers. In other related aspects, the liposome formulation encapsulates an API with components that undergo a physical transition at elevated temperature which releases the API over a period of hours to weeks.

In some aspects, the liposome formulation comprises sphingomyelin and one or more lipids disclosed herein. In some aspects, the liposome formulation comprises optisomes.

In some aspects, the disclosure provides for a liposome formulation that includes one or more lipids selected from: N-(carbonyl-methoxypolyethylene glycol 2000)-1,2-distearoyl-sn-glycero-3-phosphoethanolamine sodium salt, (distearoyl-sn-glycero-phosphoethanolamine), MPEG (methoxy polyethylene glycol)-conjugated lipid, HSPC (hydrogenated soy phosphatidylcholine); PEG (polyethylene glycol); DSPE (distearoyl-sn-glycero-phosphoethanolamine); DSPC (distearoylphosphatidylcholine); DOPC (dioleoylphosphatidylcholine); DPPG (dipalmitoylphosphatidylglycerol); EPC (egg phosphatidylcholine); DOPS (dioleoylphosphatidylserine); POPC (palmitoyloleoylphosphatidylcholine); SM (sphingomyelin); MPEG (methoxy polyethylene glycol); DMPC (dimyristoyl phosphatidylcholine); DMPG (dimyristoyl phosphatidylglycerol); DSPG (distearoylphosphatidylglycerol); DEPC (dierucoylphosphatidylcholine); DOPE (dioleoly-sn-glycero-phophoethanolamine) cholesteryl sulphate (CS), dipalmitoylphosphatidylglycerol (DPPG), DOPC (dioleoly-sn-glycero-phosphatidylcholine) or any combination thereof.

In some aspects, the disclosure provides for a liposome formulation comprising phospholipid, cholesterol and a PEG-ylated lipid in a molar ratio of 56:38:5. In some aspects, the liposome formulation's overall lipid content is from 2-16 mg/mL. In some aspects, the disclosure provides for a liposome formulation comprising a lipid containing a phosphatidylcholine functional group, a lipid containing an ethanolamine functional group and a PEG-ylated lipid. In some aspects, the disclosure provides for a liposome formulation comprising a lipid containing a phosphatidylcholine functional group, a lipid containing an ethanolamine functional group and a PEG-ylated lipid in a molar ratio of 3:0.015:2 respectively. In some aspects, the disclosure provides for a liposome formulation comprising a lipid containing a phosphatidylcholine functional group, cholesterol and a PEG-ylated lipid. In some aspects, the disclosure provides for a liposome formulation comprising a lipid containing a phosphatidylcholine functional group and cholesterol. In some aspects, the PEG-ylated lipid is PEG-2000-DSPE. In some aspects, the disclosure provides for a liposome formulation comprising DPPG, soy PC, MPEG-DSPE lipid conjugate and cholesterol.

In some aspects, the disclosure provides for a liposome formulation comprising one or more lipids containing a phosphatidylcholine functional group and one or more lipids containing an ethanolamine functional group. In some aspects, the disclosure provides for a liposome formulation comprising one or more: lipids containing a phosphatidylcholine functional group, lipids containing an ethanolamine functional group, and sterols, e.g. cholesterol. In some aspects, the liposome formulation comprises DOPC/DEPC; and DOPE.

In some aspects, the disclosure provides for a liposome formulation further comprising one or more pharmaceutical excipients, e.g. sucrose and/or glycine.

In some aspects, the disclosure provides for a liposome formulation that is either unilamellar or multilamellar in structure. In some aspects, the disclosure provides for a liposome formulation that comprises multi-vesicular particles and/or foam-based particles. In some aspects, the disclosure provides for a liposome formulation that are larger in relative size to common nanoparticles and about 150 to 250 nm in size. In some aspects, the liposome formulation is a lyophilized powder.

In some aspects, the disclosure provides for a liposome formulation that is made and loaded with ceDNA vectors disclosed or described herein, by adding a weak base to a mixture having the isolated ceDNA outside the liposome. This addition increases the pH outside the liposomes to approximately 7.3 and drives the API into the liposome. In some aspects, the disclosure provides for a liposome formulation having a pH that is acidic on the inside of the liposome. In such cases the inside of the liposome can be at pH 4-6.9, and more preferably pH 6.5. In other aspects, the disclosure provides for a liposome formulation made by using intra-liposomal drug stabilization technology. In such cases, polymeric or non-polymeric highly charged anions and intra-liposomal trapping agents are utilized, e.g. polyphosphate or sucrose octasulfate.

In other aspects, the disclosure provides for a liposome formulation comprising phospholipids, lecithin, phosphatidylcholine and phosphatidylethanolamine. In some embodiments, the liposomal formulation is a formulation described in the following Table 7.

TABLE 7
Exemplary liposomal formulations.

| Composition | PH | Composition | PH |
|---|---|---|---|
| MPEG-DSPE (3.19 mg/mL) | 6.5 | DSPC (28.16 mg/mL) | 4.9-6.0 |
| HSPC (9.58 mg/mL) | | Cholesterol (6.72 mg/mL) | |
| Cholesterol (3.19 mg/mL) | | | |
| DOPC (5.7 mg/mL) | 5.5-8.5 | Egg phosphatidylcholine: | 7.8 |
| Cholesterol (4.4 mg/mL) | | cholesterol (55:45 molar | |
| Triolein (1.2 mg/mL) | | ratio)[reconstit. from lyophilizate in | |
| DPPG (1.0 mg/mL) | | sodium carbonate buffer] | |
| DOPS:POPC (3:7 molar ratio) | 4.5-7.0 | Sphingomyelin (2.37 mg/mL, 73.5 | 7.2-7.6 |
| 1 g total lipid/vial [reconstit. | | mg/31 mL) | |
| from lyophilizate 0.9% NaCl] | | Cholesterol (0.95 mg/mL, | |
| | | 29.5 mg/31 mL) [reconstit. from | |
| | | lyophilizate in sodium phos. soln.] | |
| DSPC (6.81 mg/mL) | 6.8-7.6 | DMPC (3.4 mg/ml) | 5.0-7.0 |
| Cholesterol (2.22 mg/mL) | | DMPG (1.5 mg/ml) | |
| MPEG-2000-DSPE (0.12 mg/mL) | | in a 7:3 molar ratio | |
| HSPC (17.75 mg/mL, | 5.0-6.0 | Sodium cholesteryl sulfate (2.64 | |
| 213 mg/12 mL) | | mg/mL) [reconstit. from | |
| Cholesterol (4.33 mg/mL, | | lyophilizate in sterile water] | |
| 52 mg/12 mL) | | | |
| DSPG (7.0 mg/mL, | | | |
| 84 mg/12 mL) [reconstit. from | | | |
| lyophilizate in sterile water] | | | |
| DMPC and EPG | | DOPC (4.2 mg/mL) | 5.0-8.0 |
| (1:8 molar ratio) [reconstit. from | | Cholesterol (3.3 mg/mL) | |
| lyophilizate in sterile water] | | DPPG (0.9 mg/mL) | |
| | | Tricaprylin (0.3 mg/mL) | |
| | | Triolein (0.1 mg/mL) | |
| Cholesterol (4.7 mg/mL) | 5.8-7.4 | DOPC:DOPE | |
| DPPG (0.9 mg/mL) | | (75:25 molar ratio) | |
| Tricaprylin (2.0 mg/mL) | | | |
| DEPC (8.2 mg/mL) | | | |

In some aspects, the disclosure provides for a lipid nanoparticle comprising ceDNA and an ionizable lipid. For example, a lipid nanoparticle formulation that is made and loaded with ceDNA obtained by the process as disclosed in International Application PCT/US2018/050042, filed on Sep. 7, 2018, which is incorporated herein. This can be accomplished by high energy mixing of ethanolic lipids with aqueous ceDNA at low pH which protonates the ionizable lipid and provides favorable energetics for ceDNA/lipid association and nucleation of particles. The particles can be further stabilized through aqueous dilution and removal of the organic solvent. The particles can be concentrated to the desired level.

Generally, the lipid particles are prepared at a total lipid to ceDNA (mass or weight) ratio of from about 10:1 to 30:1. In some embodiments, the lipid to ceDNA ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and ceDNA can be adjusted to provide a desired N/P ratio, for example, N/P ratio of 3, 4, 5, 6, 7, 8, 9, 10 or higher. Generally, the lipid particle formulation's overall lipid content can range from about 5 mg/ml to about 30 mg/mL.

The ionizable lipid is typically employed to condense the nucleic acid cargo, e.g., ceDNA at low pH and to drive membrane association and fusogenicity. Generally, ionizable lipids are lipids comprising at least one amino group that is positively charged or becomes protonated under acidic conditions, for example at pH of 6.5 or lower. Ionizable lipids are also referred to as cationic lipids herein.

Exemplary ionizable lipids are described in PCT patent publications WO2015/095340, WO2015/199952, WO2018/011633, WO2017/049245, WO2015/061467, WO2012/040184, WO2012/000104, WO2015/074085, WO2016/081029, WO2017/004143, WO2017/075531, WO2017/117528, WO2011/022460, WO2013/148541, WO2013/116126, WO2011/153120, WO2012/044638, WO2012/054365, WO2011/090965, WO2013/016058, WO2012/162210, WO2008/042973, WO2010/129709, WO2010/144740, WO2012/099755, WO2013/049328, WO2013/086322, WO2013/086373, WO2011/071860, WO2009/132131, WO2010/048536, WO2010/088537, WO2010/054401, WO2010/054406, WO2010/054405, WO2010/054384, WO2012/016184, WO2009/086558, WO2010/042877, WO2011/000106, WO2011/000107, WO2005/120152, WO2011/141705, WO2013/126803, WO2006/007712, WO2011/038160, WO2005/121348, WO2011/066651, WO2009/127060, WO2011/141704, WO2006/069782, WO2012/031043, WO2013/006825, WO2013/033563, WO2013/089151, WO2017/099823, WO2015/095346, and WO2013/086354, and US patent publications US2016/0311759, US2015/0376115, US2016/0151284, US2017/0210697, US2015/0140070, US2013/0178541, US2013/0303587, US2015/0141678, US2015/0239926, US2016/0376224, US2017/0119904, US2012/0149894, US2015/0057373, US2013/0090372, US2013/0274523, US2013/0274504, US2013/0274504, US2009/0023673, US2012/0128760, US2010/0324120, US2014/0200257, US2015/0203446, US2018/0005363, US2014/0308304, US2013/0338210, US2012/0101148, US2012/0027796, US2012/0058144, US2013/0323269, US2011/0117125, US2011/0256175, US2012/0202871, US2011/0076335, US2006/0083780, US2013/0123338, US2015/0064242, US2006/0051405, US2013/0065939, US2006/0008910, US2003/0022649, US2010/0130588, US2013/0116307, US2010/0062967, US2013/0202684, US2014/0141070, US2014/0255472, US2014/0039032, US2018/0028664, US2016/0317458, and US2013/0195920, the contents of all of which are incorporated herein by reference in their entirety.

In some embodiments, the ionizable lipid is MC3 (6Z,9Z,28Z,31Z)-heptatriaconta-6,9,28,31-tetraen-19-yl-4-(dimethylamino) butanoate (DLin-MC3-DMA or MC3) having the following structure:

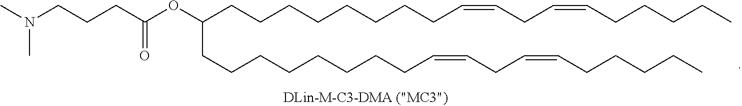 [SMILES: CCCCC/C=C\C/C=C\CCCCCCCCC(CCCCCCCC/C=C\C/C=C\CCCCC)OC(=O)CCCN(C)C]

The lipid DLin-MC3-DMA is described in Jayaraman et al., Angew. Chem. Int. Ed Engl. (2012), 51(34): 8529-8533, content of which is incorporated herein by reference in its entirety.

In some embodiments, the ionizable lipid is the lipid ATX-002 having the following structure:

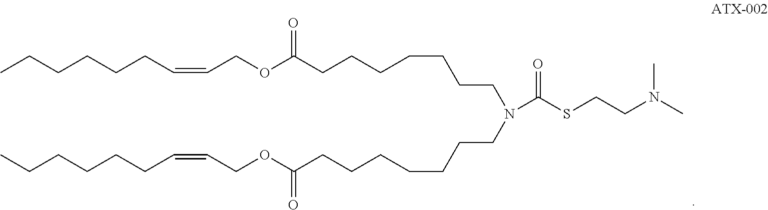 [SMILES: CCCCCC/C=C\COC(=O)CCCCCCCN(CCCCCCCC(=O)OC/C=C\CCCCCC)C(=O)SCCN(C)C]

The lipid ATX-002 is described in WO2015/074085, content of which is incorporated herein by reference in its entirety.

In some embodiments, the ionizable lipid is (13Z,16Z)—N,N-dimethyl-3-nonyldocosa-13,16-dien-1-amine (Compound 32) having the following structure:

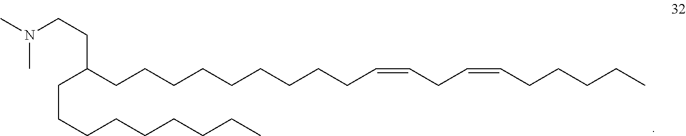 [SMILES: CCCCC/C=C\C/C=C\CCCCCCCCCC(CCCCCCCCC)CCN(C)C]

Compound 32 is described in WO2012/040184, content of which is incorporated herein by reference in its entirety.

In some embodiments, the ionizable lipid is Compound 6 or Compound 22 having the following structure:

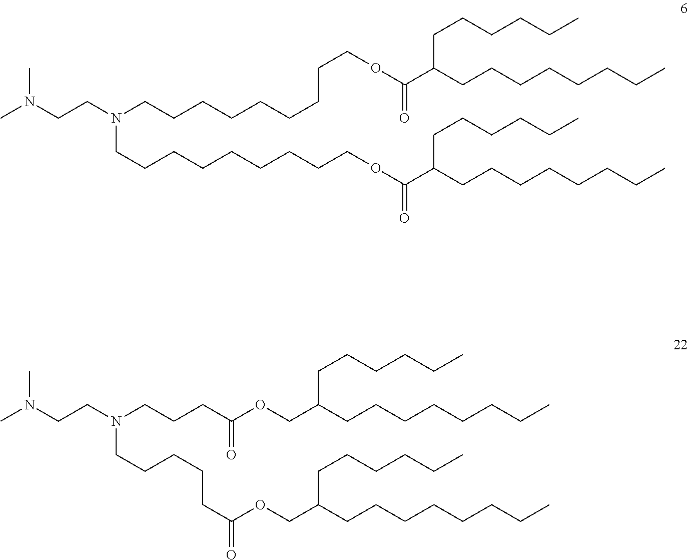

Compounds 6 and 22 are described in WO2015/199952, content of which is incorporated herein by reference in its entirety.

Without limitations, ionizable lipid can comprise 20-90% (mol) of the total lipid present in the lipid nanoparticle. For example, ionizable lipid molar content can be 20-70% (mol), 30-60% (mol) or 40-50% (mol) of the total lipid present in the lipid nanoparticle. In some embodiments, ionizable lipid comprises from about 50 mol % to about 90 mol % of the total lipid present in the lipid nanoparticle.

In some aspects, the lipid nanoparticle can further comprise a non-cationic lipid. Non-ionic lipids include amphipathic lipids, neutral lipids and anionic lipids. Accordingly, the non-cationic lipid can be a neutral uncharged, zwitterionic, or anionic lipid. Non-cationic lipids are typically employed to enhance fusogenicity.

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having Cio-C24 carbon chains, e.g., lauroyl, myristoyl, palmitoyl, stearoyl, or oleoyl.

Other examples of non-cationic lipids suitable for use in the lipid nanoparticles include nonphosphorous lipids such as, e.g., stearylamine, dodecylamine, hexadecylamine, acetyl palmitate, glycerolricinoleate, hexadecyl stereate, isopropyl myristate, amphoteric acrylic polymers, triethanolamine-lauryl sulfate, alkyl-aryl sulfate polyethyloxylated fatty acid amides, dioctadecyldimethyl ammonium bromide, ceramide, sphingomyelin, and the like.

In some embodiments, the non-cationic lipid is a phospholipid. In some embodiments, the non-cationic lipid is selected from DSPC, DPPC, DMPC, DOPC, POPC, DOPE, and SM. In some preferred embodiments, the non-cationic lipid is DPSC.

Exemplary non-cationic lipids are described in PCT Publication WO2017/099823 and US patent publication US2018/0028664, the contents of both of which are incorporated herein by reference in their entirety. In some examples, the non-cationic lipid is oleic acid or a compound of

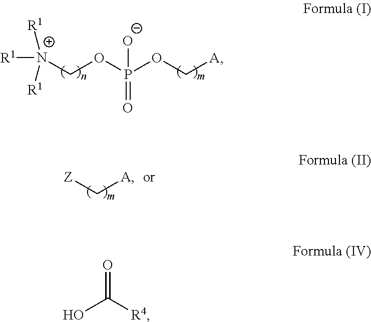

as defined in US2018/0028664, the content of which is incorporated herein by reference in its entirety.

The non-cationic lipid can comprise 0-30% (mol) of the total lipid present in the lipid nanoparticle. For example, the non-cationic lipid content is 5-20% (mol) or 10-15% (mol) of the total lipid present in the lipid nanoparticle. In various embodiments, the molar ratio of ionizable lipid to the neutral lipid ranges from about 2:1 to about 8:1.

In some embodiments, the lipid nanoparticles do not comprise any phospholipids.

In some aspects, the lipid nanoparticle can further comprise a component, such as a sterol, to provide membrane integrity.

One exemplary sterol that can be used in the lipid nanoparticle is cholesterol and derivatives thereof. Non-limiting examples of cholesterol derivatives include polar analogues such as 5a-cholestanol, 5β-coprostanol, cholesteryl-(2′-hydroxy)-ethyl ether, cholesteryl-(4′-hydroxy)-butyl ether, and 6-ketocholestanol; non-polar analogues such as 5a-cholestane, cholestenone, 5a-cholestanone, 5β-cholestanone, and cholesteryl decanoate; and mixtures thereof. In some embodiments, the cholesterol derivative is a polar analogue such as cholesteryl-(4′-hydroxy)-butyl ether.

Exemplary cholesterol derivatives are described in PCT publication WO2009/127060 and US patent publication US2010/0130588, contents of both of which are incorporated herein by reference in their entirety.

The component providing membrane integrity, such as a sterol, can comprise 0-50% (mol) of the total lipid present in the lipid nanoparticle. In some embodiments, such a component is 20-50% (mol) 30-40% (mol) of the total lipid content of the lipid nanoparticle.

In some aspects, the lipid nanoparticle can further comprise a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid.

Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-O-(2′,3′-di(tetradecanoyloxy)propyl-1-O-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, and US2017/0119904, the contents of all of which are incorporated herein by reference in their entirety.

In some embodiments, a PEG-lipid is a compound of

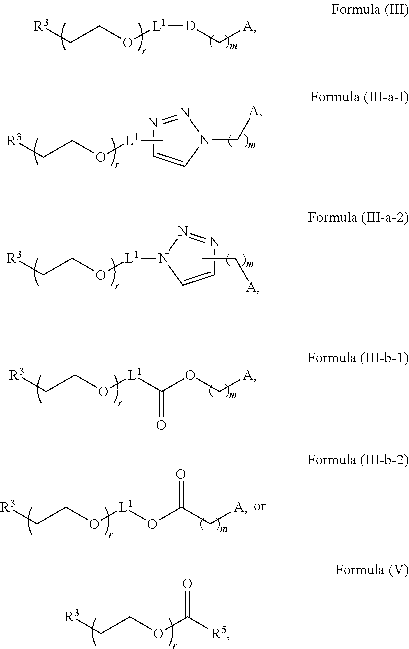

as defined in US2018/0028664, the content of which is incorporated herein by reference in its entirety.

In some embodiments, a PEG-lipid is of

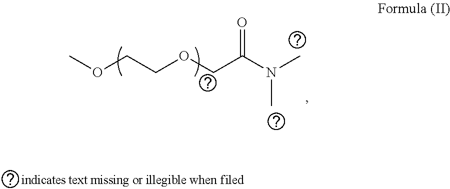

as defined in US20150376115 or in US2016/0376224, the content of both of which is incorporated herein by reference in its entirety.

The PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In some examples, the PEG-lipid can be selected from the group consisting of PEG-DMG, 1,2-dimyristoyl-sn-glycero-3-phosphoethanolamine-N-[methoxy(polyethylene glycol)-2000],

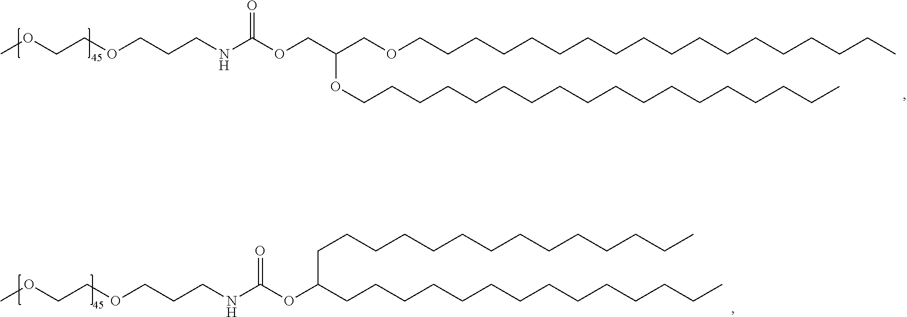

Lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (CPL) conjugates can be used in place of or in addition to the PEG-lipid.

Exemplary conjugated lipids, i.e., PEG-lipids, (POZ)-lipid conjugates, ATTA-lipid conjugates and cationic polymer-lipids are described in the PCT patent application publications WO1996/010392, WO1998/051278, WO2002/087541, WO2005/026372, WO2008/147438, WO2009/086558, WO2012/000104, WO2017/117528, WO2017/099823, WO2015/199952, WO2017/004143, WO2015/095346, WO2012/000104, WO2012/000104, and WO2010/006282, US patent application publications US2003/0077829, US2005/0175682, US2008/0020058, US2011/0117125, US2013/0303587, US2018/0028664, US2015/0376115, US2016/0376224, US2016/0317458, US2013/0303587, US2013/0303587, and US20110123453, and US patents U.S. Pat. Nos. 5,885,613, 6,287,591, 6,320,017, and 6,586,559, the contents of all of which are incorporated herein by reference in their entirety.

The PEG or the conjugated lipid can comprise 0-20% (mol) of the total lipid present in the lipid nanoparticle. In some embodiments, PEG or the conjugated lipid content is 0.5-10% or 2-5% (mol) of the total lipid present in the lipid nanoparticle.

Molar ratios of the ionizable lipid, non-cationic-lipid, sterol, and PEG/conjugated lipid can be varied as needed. For example, the lipid particle can comprise 30-70% ionizable lipid by mole or by total weight of the composition, 0-60% cholesterol by mole or by total weight of the composition, 0-30% non-cationic-lipid by mole or by total weight of the composition and 1-10% conjugated lipid by mole or by total weight of the composition. Preferably, the composition comprises 30-40% ionizable lipid by mole or by total weight of the composition, 40-50% cholesterol by mole or by total weight of the composition, and 10-20% non-cationic-lipid by mole or by total weight of the composition. In some other embodiments, the composition is 50-75% ionizable lipid by mole or by total weight of the composition, 20-40% cholesterol by mole or by total weight of the composition, and 5 to 10% non-cationic-lipid, by mole or by total weight of the composition and 1-10% conjugated lipid by mole or by total weight of the composition. The composition may contain 60-70% ionizable lipid by mole or by total weight of the composition, 25-35% cholesterol by mole or by total weight of the composition, and 5-10% non-cationic-lipid by mole or by total weight of the composition. The composition may also contain up to 90% ionizable lipid by mole or by total weight of the composition and 2 to 15% non-cationic lipid by mole or by total weight of the composition. The formulation may also be a lipid nanoparticle formulation, for example comprising 8-30% ionizable lipid by mole or by total weight of the composition, 5-30% non-cationic lipid by mole or by total weight of the composition, and 0-20% cholesterol by mole or by total weight of the composition; 4-25% ionizable lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% ionizable lipid by mole or by total weight of the composition, 2-30% non-cationic lipid by mole or by total weight of the composition, 1 to 15% cholesterol by mole or by total weight of the composition, 2 to 35% conjugate lipid by mole or by total weight of the composition, and 1-20% cholesterol by mole or by total weight of the composition; or even up to 90% ionizable lipid by mole or by total weight of the composition and 2-10% non-cationic lipids by mole or by total weight of the composition, or even 100% cationic lipid by mole or by total weight of the composition. In some embodiments, the lipid particle formulation comprises ionizable lipid, phospholipid, cholesterol and a PEG-ylated lipid in a molar ratio of 50:10:38.5:1.5. In some other embodiments, the lipid particle formulation comprises ionizable lipid, cholesterol and a PEG-ylated lipid in a molar ratio of 60:38.5:1.5.

In some embodiments, the lipid particle comprises ionizable lipid, non-cationic lipid (e.g. phospholipid), a sterol (e.g., cholesterol) and a PEG-ylated lipid, where the molar ratio of lipids ranges from 20 to 70 mole percent for the ionizable lipid, with a target of 40-60, the mole percent of non-cationic lipid ranges from 0 to 30, with a target of 0 to 15, the mole percent of sterol ranges from 20 to 70, with a target of 30 to 50, and the mole percent of PEG-ylated lipid ranges from 1 to 6, with a target of 2 to 5.

Lipid nanoparticles (LNPs) comprising ceDNA are disclosed in International Application PCT/US2018/050042, filed on Sep. 7, 2018, which is incorporated herein in its entirety and envisioned for use in the methods and compostions as disclosed herein.

Lipid nanoparticle particle size can be determined by quasi-elastic light scattering using a Malvern Zetasizer Nano ZS (Malvern, UK) and is approximately 50-150 nm diameter, approximately 55-95 nm diameter, or approximately 70-90 nm diameter.

The pKa of formulated cationic lipids can be correlated with the effectiveness of the LNPs for delivery of nucleic acids (see Jayaraman et al, Angewandte Chemie, International Edition (2012), 51(34), 8529-8533; Semple et al, Nature Biotechnology 28, 172-176 (2010), both of which are incorporated by reference in their entirety). The preferred range of pKa is ˜5 to ˜7. The pKa of each cationic lipid is determined in lipid nanoparticles using an assay based on fluorescence of 2-(p-toluidino)-6-napthalene sulfonic acid (TNS). Lipid nanoparticles comprising of cationic lipid/DSPC/cholesterol/PEG-lipid (50/10/38.5/1.5 mol %) in PBS at a concentration of 0.4 mM total lipid can be prepared using the in-line process as described herein and elsewhere. TNS can be prepared as a 100 μM stock solution in distilled water. Vesicles can be diluted to 24 μM lipid in 2 mL of buffered solutions containing, 10 mM HEPES, 10 mM MES, 10 mM ammonium acetate, 130 mM NaCl, where the pH ranges from 2.5 to 11. An aliquot of the TNS solution can be added to give a final concentration of 1 μM and following vortex mixing fluorescence intensity is measured at room temperature in a SLM Aminco Series 2 Luminescence Spectrophotometer using excitation and emission wavelengths of 321 nm and 445 nm. A sigmoidal best fit analysis can be applied to the fluorescence data and the pKa is measured as the pH giving rise to half-maximal fluorescence intensity.

Relative activity can be determined by measuring luciferase expression in the liver 4 hours following administration via tail vein injection. The activity is compared at a dose of 0.3 and 1.0 mg ceDNA/kg and expressed as ng luciferase/g liver measured 4 hours after administration.

Without limitations, a lipid nanoparticle of the invention includes a lipid formulation that can be used to deliver a capsid-free, non-viral DNA vector to a target site of interest (e.g., cell, tissue, organ, and the like). Generally, the lipid nanoparticle comprises capsid-free, non-viral DNA vector and an ionizable lipid or a salt thereof.

In some embodiments, the lipid particle comprises ionizable lipid/non-cationic-lipid/sterol/conjugated lipid at a molar ratio of 50:10:38.5:1.5.

In other aspects, the disclosure provides for a lipid nanoparticle formulation comprising phospholipids, lecithin, phosphatidylcholine and phosphatidylethanolamine.

In some embodiments, one or more additional compounds can also be included. Those compounds can be administered separately or the additional compounds can be included in the lipid nanoparticles of the invention. In other words, the lipid nanoparticles can contain other compounds in addition to the ceDNA or at least a second ceDNA, different than the first. Without limitations, other additional compounds can be selected from the group consisting of small or large organic or inorganic molecules, monosaccharides, disaccharides, trisaccharides, oligosaccharides, polysaccharides, peptides, proteins, peptide analogs and derivatives thereof, peptidomimetics, nucleic acids, nucleic acid analogs and derivatives, an extract made from biological materials, or any combinations thereof.

In some embodiments, the one or more additional compound can be a therapeutic agent. The therapeutic agent can be selected from any class suitable for the therapeutic objective. In other words, the therapeutic agent can be selected from any class suitable for the therapeutic objective. In other words, the therapeutic agent can be selected according to the treatment objective and biological action desired. For example, if the ceDNA within the LNP is useful for treating cancer, the additional compound can be an anti-cancer agent (e.g., a chemotherapeutic agent, a targeted cancer therapy (including, but not limited to, a small molecule, an antibody, or an antibody-drug conjugate). In another example, if the LNP containing the ceDNA is useful for treating an infection, the additional compound can be an antimicrobial agent (e.g., an antibiotic or antiviral compound). In yet another example, if the LNP containing the ceDNA is useful for treating an immune disease or disorder, the additional compound can be a compound that modulates an immune response (e.g., an immunosuppressant, immunostimulatory compound, or compound modulating one or more specific immune pathways). In some embodiments, different cocktails of different lipid nanoparticles containing different compounds, such as a ceDNA encoding a different protein or a different compound, such as a therapeutic may be used in the compositions and methods of the invention.

In some embodiments, the additional compound is an immune modulating agent. For example, the additional compound is an immunosuppressant. In some embodiments, the additional compound is immunestimulatory.

Also provided herein is a pharmaceutical composition comprising the lipid nanoparticle and a pharmaceutically acceptable carrier or excipient.

In some aspects, the disclosure provides for a lipid nanoparticle formulation further comprising one or more pharmaceutical excipients. In some embodiments, the lipid nanoparticle formulation further comprises sucrose, tris, trehalose and/or glycine.

Generally, the lipid nanoparticles of the invention have a mean diameter selected to provide an intended therapeutic effect. Accordingly, in some aspects, the lipid nanoparticle has a mean diameter from about 30 nm to about 150 nm, more typically from about 50 nm to about 150 nm, more typically about 60 nm to about 130 nm, more typically about 70 nm to about 110 nm, most typically about 85 nm to about 105 nm, and preferably about 100 nm. In some aspects, the disclosure provides for lipid particles that are larger in relative size to common nanoparticles and about 150 to 250 nm in size. Lipid nanoparticle particle size can be determined by quasi-elastic light scattering using, for example, a Malvern Zetasizer Nano ZS (Malvern, UK) system.

Depending on the intended use of the lipid particles, the proportions of the components can be varied and the delivery efficiency of a particular formulation can be measured using, for example, an endosomal release parameter (ERP) assay.

The ceDNA can be complexed with the lipid portion of the particle or encapsulated in the lipid position of the lipid nanoparticle. In some embodiments, the ceDNA can be fully encapsulated in the lipid position of the lipid nanoparticle, thereby protecting it from degradation by a nuclease, e.g., in an aqueous solution. In some embodiments, the ceDNA in the lipid nanoparticle is not substantially degraded after exposure of the lipid nanoparticle to a nuclease at 37° C. for at least about 20, 30, 45, or 60 minutes. In some embodiments, the ceDNA in the lipid nanoparticle is not substantially degraded after incubation of the particle in serum at 37° C. for at least about 30, 45, or 60 minutes or at least about 2, 3, 4, 5, 6, 7, 8, 9, 10, 12, 14, 16, 18, 20, 22, 24, 26, 28, 30, 32, 34, or 36 hours.

In certain embodiments, the lipid nanoparticles are substantially non-toxic to a subject, e.g., to a mammal such as a human.

In some aspects, the lipid nanoparticle formulation is a lyophilized powder.

In some embodiments, lipid nanoparticles are solid core particles that possess at least one lipid bilayer. In other embodiments, the lipid nanoparticles have a non-bilayer structure, i.e., a non-lamellar (i.e., non-bilayer) morphology. Without limitations, the non-bilayer morphology can include, for example, three dimensional tubes, rods, cubic symmetries, etc. The non-lamellar morphology (i.e., non-bilayer structure) of the lipid particles can be determined using analytical techniques known to and used by those of skill in the art. Such techniques include, but are not limited to, Cryo-Transmission Electron Microscopy (“Cryo-TEM”), Differential Scanning calorimetry (“DSC”), X-Ray Diffraction, and the like. For example, the morphology of the lipid nanoparticles (lamellar vs. non-lamellar) can readily be assessed and characterized using, e.g., Cryo-TEM analysis as described in US2010/0130588, the content of which is incorporated herein by reference in its entirety.

In some further embodiments, the lipid nanoparticles having a non-lamellar morphology are electron dense.

In some aspects, the disclosure provides for a lipid nanoparticle that is either unilamellar or multilamellar in structure. In some aspects, the disclosure provides for a lipid nanoparticle formulation that comprises multi-vesicular particles and/or foam-based particles.

By controlling the composition and concentration of the lipid components, one can control the rate at which the lipid conjugate exchanges out of the lipid particle and, in turn, the rate at which the lipid nanoparticle becomes fusogenic. In addition, other variables including, e.g., pH, temperature, or ionic strength, can be used to vary and/or control the rate at which the lipid nanoparticle becomes fusogenic. Other methods which can be used to control the rate at which the lipid nanoparticle becomes fusogenic will be apparent to those of ordinary skill in the art based on this disclosure. It will also be apparent that by controlling the composition and concentration of the lipid conjugate, one can control the lipid particle size.

The pKa of formulated cationic lipids can be correlated with the effectiveness of the LNPs for delivery of nucleic acids (see Jayaraman et al, Angewandte Chemie, International Edition (2012), 51(34), 8529-8533; Semple et al, Nature Biotechnology 28, 172-176 (2010), both of which are incorporated by reference in their entirety). The preferred range of pKa is ˜5 to ˜7. The pKa of the cationic lipid can be determined in lipid nanoparticles using an assay based on fluorescence of 2-(p-toluidino)-6-napthalene sulfonic acid (TNS).

Encapsulation of ceDNA in lipid particles can be determined by performing a membrane-impermeable fluorescent dye exclusion assay, which uses a dye that has enhanced fluorescence when associated with nucleic acid, for example, an Oligreen® assay or PicoGreen® assay. Generally, encapsulation is determined by adding the dye to the lipid particle formulation, measuring the resulting fluorescence, and comparing it to the fluorescence observed upon addition of a small amount of nonionic detergent. Detergent-mediated disruption of the lipid bilayer releases the encapsulated ceDNA, allowing it to interact with the membrane-impermeable dye. Encapsulation of ceDNA can be calculated as E=(I₀−I)/I₀, where I and I₀ refers to the fluorescence intensities before and after the addition of detergent.

### IX. Methods of Delivering ceDNA Vectors

In some embodiments, a ceDNA vector can be delivered to a target cell in vitro or in vivo by various suitable methods. ceDNA vectors alone can be applied or injected. CeDNA vectors can be delivered to a cell without the help of a transfection reagent or other physical means. Alternatively, ceDNA vectors can be delivered using any art-known transfection reagent or other art-known physical means that facilitates entry of DNA into a cell, e.g., liposomes, alcohols, polylysine-rich compounds, arginine-rich compounds, calcium phosphate, microvesicles, microinjection, electroporation and the like.

In contrast, transductions with capsid-free AAV vectors disclosed herein can efficiently target cell and tissue-types that are difficult to transduce with conventional AAV virions using various delivery reagent.

In another embodiment, a ceDNA vector is administered to the CNS (e.g., to the brain or to the eye). The ceDNA vector may be introduced into the spinal cord, brainstem (medulla oblongata, pons), midbrain (hypothalamus, thalamus, epithalamus, pituitary gland, substantia nigra, pineal gland), cerebellum, telencephalon (corpus striatum, cerebrum including the occipital, temporal, parietal and frontal lobes, cortex, basal ganglia, hippocampus and portaamygdala), limbic system, neocortex, corpus striatum, cerebrum, and inferior colliculus. The ceDNA vector may also be administered to different regions of the eye such as the retina, cornea and/or optic nerve. The ceDNA vector may be delivered into the cerebrospinal fluid (e.g., by lumbar puncture). The ceDNA vector may further be administered intravascularly to the CNS in situations in which the blood-brain barrier has been perturbed (e.g., brain tumor or cerebral infarct).

In some embodiments, the ceDNA vector can be administered to the desired region(s) of the CNS by any route known in the art, including but not limited to, intrathecal, intra-ocular, intracerebral, intraventricular, intravenous (e.g., in the presence of a sugar such as mannitol), intranasal, intra-aural, intra-ocular (e.g., intra-vitreous, sub-retinal, anterior chamber) and peri-ocular (e.g., sub-Tenon's region) delivery as well as intramuscular delivery with retrograde delivery to motor neurons.

In some embodiments, the ceDNA vector is administered in a liquid formulation by direct injection (e.g., stereotactic injection) to the desired region or compartment in the CNS. In other embodiments, the ceDNA vector can be provided by topical application to the desired region or by intra-nasal administration of an aerosol formulation. Administration to the eye may be by topical application of liquid droplets. As a further alternative, the ceDNA vector can be administered as a solid, slow-release formulation (see, e.g., U.S. Pat. No. 7,201,898). In yet additional embodiments, the ceDNA vector can used for retrograde transport to treat, ameliorate, and/or prevent diseases and disorders involving motor neurons (e.g., amyotrophic lateral sclerosis (ALS); spinal muscular atrophy (SMA), etc.). For example, the ceDNA vector can be delivered to muscle tissue from which it can migrate into neurons.

### X. Additional Uses of the ceDNA Vectors

The compositions and ceDNA vectors provided herein can be used to gene edit a target gene for various purposes. In some embodiments, the resulting transgene encodes a protein or functional RNA that is intended to be used for research purposes, e.g., to create a somatic transgenic animal model harboring the transgene, e.g., to study the function of the transgene product. In another example, the transgene encodes a protein or functional RNA that is intended to be used to create an animal model of disease. In some embodiments, the resulting transgene encodes one or more peptides, polypeptides, or proteins, which are useful for the treatment, prevention, or amelioration of disease states or disorders in a mammalian subject. The resulting transgene can be transferred (e.g., expressed in) to a subject in a sufficient amount to treat a disease associated with reduced expression, lack of expression or dysfunction of the gene. In some embodiments the resulting transgene can be expressed in a subject in a sufficient amount to treat a disease associated with increased expression, activity of the gene product, or inappropriate upregulation of a gene that the resulting transgene suppresses or otherwise causes the expression of which to be reduced. In yet other embodiments, the resulting transgene replaces or supplements a defective copy of the native gene. It will be appreciated by one of ordinary skill in the art that the transgene may not be an open reading frame of a gene to be transcribed itself; instead it may be a promoter region or repressor region of a target gene, and the ceDNA gene editing vector may modify such region with the outcome of so modulating the expression of a gene of interest.

In some embodiments, the transgene encodes a protein or functional RNA that is intended to be used to create an animal model of disease. In some embodiments, the transgene encodes one or more peptides, polypeptides, or proteins, which are useful for the treatment or prevention of disease states in a mammalian subject. The transgene or donor sequence can be transferred (e.g., expressed in) to a patient in a sufficient amount to treat a disease associated with reduced expression, lack of expression or dysfunction of the gene. In some embodiments, the transgene is a gene editing molecule (e.g., nuclease). In certain embodiments, the nuclease is a CRISPR-associated nuclease (Cas nuclease).

### XI. Methods of Use

The ceDNA vector for gene editing as disclosed herein can also be used in a method for the delivery of a nucleotide sequence of interest (e.g., a gene editing molecule, e.g., a nuclease or a guide sequence) to a target cell (e.g., a host cell). The method may in particular be a method for delivering a gene editing molecule to a cell of a subject in need thereof and for editing a target gene of interest. The invention allows for the in vivo expression of a gene editing molecule, e.g., a nuclease or a guide sequence encoded in the ceDNA vector in a cell in a subject such that therapeutic effect of the gene editing machinery occurs. These results are seen with both in vivo and in vitro modes of ceDNA vector delivery.

In addition, the invention provides a method for the delivery of a gene editing molecule in a cell of a subject in need thereof, comprising multiple administrations of the ceDNA vector of the invention comprising said nucleic acid of interest. Since the ceDNA vector of the invention does not induce an immune response like that typically observed against encapsidated viral vectors, such a multiple administration strategy will likely have greater success in a ceDNA-based system.

The ceDNA vector nucleic acid(s) are administered in sufficient amounts to transfect the cells of a desired tissue and to provide sufficient levels of gene transfer and expression without undue adverse effects. Conventional and pharmaceutically acceptable routes of administration include, but are not limited to, intravenous (e.g., in a liposome formulation), direct delivery to the selected organ (e.g., intraportal delivery to the liver), intramuscular, and other parental routes of administration. Routes of administration may be combined, if desired.

ceDNA delivery is not limited to ceDNA vector delivery of all nucleotides encoding gene editing components. For example, ceDNA vectors as described herein may be used with other delivery systems provided to provide a portion of the gene editing components. One non-limiting example of a system that may be combined with ceDNA vectors in accordance with the present disclosure includes systems which separately deliver Cas9 to a host cell in need of treatment or gene editing. In certain embodiments, Cas9 may be delivered in a nanoparticle such as those described in Lee et al., Nanoparticle delivery of Cas9 ribonucleotideprotein and donor DNA in vivo induces homology-directed DNA repair, Nature Biomedical Engineering, 2017 (herein incorporated by reference in its entirety), while other components, such as a donor sequence are provided by ceDNA.

The invention also provides for a method of treating a disease in a subject comprising introducing into a target cell in need thereof (in particular a muscle cell or tissue) of the subject a therapeutically effective amount of a ceDNA vector, optionally with a pharmaceutically acceptable carrier. While the ceDNA vector can be introduced in the presence of a carrier, such a carrier is not required. The ceDNA vector implemented comprises a nucleotide sequence of interest useful for treating the disease. In particular, the ceDNA vector may comprise a desired exogenous DNA sequence operably linked to control elements capable of directing transcription of the desired polypeptide, protein, or oligonucleotide encoded by the exogenous DNA sequence when introduced into the subject. The ceDNA vector can be administered via any suitable route as provided above, and elsewhere herein.

The compositions and vectors provided herein can be used to deliver a transgene for various purposes. In some embodiments, the transgene encodes a protein or functional RNA that is intended to be used for research purposes, e.g., to create a somatic transgenic animal model harboring the transgene, e.g., to study the function of the transgene product. In another example, the transgene encodes a protein or functional RNA that is intended to be used to create an animal model of disease. In some embodiments, the transgene encodes one or more peptides, polypeptides, or proteins, which are useful for the treatment or prevention of disease states in a mammalian subject. The transgene can be transferred (e.g., expressed in) to a patient in a sufficient amount to treat a disease associated with reduced expression, lack of expression or dysfunction of the gene. In some embodiments, the transgene is a gene editing molecule (e.g., nuclease). In certain embodiments, the nuclease is a CRISPR-associated nuclease (Cas nuclease).

In principle, the expression cassette can include a nucleic acid or nuclease targeting any gene that encodes a protein or polypeptide that is either reduced or absent due to a mutation or which conveys a therapeutic benefit when overexpressed is considered to be within the scope of the invention. The ceDNA vector comprises a template nucleotide sequence used as a correcting DNA strand to be inserted after a double-strand break provided by a meganuclease- or zinc finger nuclease. The ceDNA vector can comprise a template nucleotide sequence used as a correcting DNA strand to be inserted after a double-strand break provided by a meganuclease- or zinc finger nuclease. Preferably, noninserted bacterial DNA is not present and preferably no bacterial DNA is present in the ceDNA compositions provided herein.

A ceDNA vector delivery for gene editing is not limited to one species of ceDNA vector. As such, in another aspect, multiple ceDNA vectors comprising different donor sequences and/or gene editing sequences can be delivered simultaneously or sequentially to the target cell, tissue, organ, or subject. Therefore, this strategy can allow for the gene-editing of multiple genes simultaneously. It is also possible to separate different portions of the gene editing functionality into separate ceDNA vectors which can be administered simultaneously or at different times, and can be separately regulatable. Delivery can also be performed multiple times and, importantly for gene therapy in the clinical setting, in subsequent increasing or decreasing doses, given the lack of an anti-capsid host immune response due to the absence of a viral capsid. It is anticipated that no anti-capsid response will occur as there is no capsid.

The invention also provides for a method of treating a disease in a subject comprising introducing into a target cell in need thereof (in particular a muscle cell or tissue) of the subject a therapeutically effective amount of a ceDNA vector for gene editing, optionally with a pharmaceutically acceptable carrier. While the ceDNA vector can be introduced in the presence of a carrier, such a carrier is not required. The ceDNA vector implemented comprises a nucleotide sequence of interest useful for treating the disease. In particular, the ceDNA vector may comprise a desired exogenous DNA sequence operably linked to control elements capable of directing transcription of the desired polypeptide, protein, or oligonucleotide encoded by the exogenous DNA sequence when introduced into the subject. The ceDNA vector can be administered via any suitable route as provided above, and elsewhere herein.

### XII. Methods of Treatment

The technology described herein also demonstrates methods for making, as well as methods of using the disclosed ceDNA vectors in a variety of ways, including, for example, ex situ, in vitro and in vivo applications, methodologies, diagnostic procedures, and/or gene therapy regimens.

Provided herein is a method of treating a disease or disorder in a subject comprising introducing into a target cell in need thereof (for example, a muscle cell or tissue, or other affected cell type) of the subject a therapeutically effective amount of a gene editing ceDNA vector, optionally with a pharmaceutically acceptable carrier. While the ceDNA vector can be introduced in the presence of a carrier, such a carrier is not required. The ceDNA vector implemented comprises a nucleotide sequence of interest useful for treating the disease. In particular, the ceDNA vector may comprise a desired exogenous DNA sequence operably linked to control elements capable of directing transcription of the desired polypeptide, protein, or oligonucleotide encoded by the exogenous DNA sequence when introduced into the subject. The ceDNA vector can be administered via any suitable route as provided above, and elsewhere herein.

Disclosed herein are ceDNA vector compositions and formulations that include one or more of the ceDNA vectors of the present invention together with one or more pharmaceutically-acceptable buffers, diluents, or excipients. Such compositions may be included in one or more diagnostic or therapeutic kits, for diagnosing, preventing, treating or ameliorating one or more symptoms of a disease, injury, disorder, trauma or dysfunction. In one aspect the disease, injury, disorder, trauma or dysfunction is a human disease, injury, disorder, trauma or dysfunction.

Another aspect of the technology described herein provides a method for providing a subject in need thereof with a diagnostically- or therapeutically-effective amount of a ceDNA vector, the method comprising providing to a cell, tissue or organ of a subject in need thereof, an amount of the ceDNA vector as disclosed herein; and for a time effective to enable expression of the transgene from the ceDNA vector thereby providing the subject with a diagnostically- or a therapeutically-effective amount of the protein, peptide, nucleic acid expressed by the ceDNA vector. In a further aspect, the subject is human.

Another aspect of the technology described herein provides a method for diagnosing, preventing, treating, or ameliorating at least one or more symptoms of a disease, a disorder, a dysfunction, an injury, an abnormal condition, or trauma in a subject. In an overall and general sense, the method includes at least the step of administering to a subject in need thereof one or more of the disclosed ceDNA vectors, in an amount and for a time sufficient to diagnose, prevent, treat or ameliorate the one or more symptoms of the disease, disorder, dysfunction, injury, abnormal condition, or trauma in the subject. In a further aspect, the subject is human.

Another aspect is use of the ceDNA vector as a tool for treating or reducing one or more symptoms of a disease or disease states. There are a number of inherited diseases in which defective genes are known, and typically fall into two classes: deficiency states, usually of enzymes, which are generally inherited in a recessive manner, and unbalanced states, which may involve regulatory or structural proteins, and which are typically but not always inherited in a dominant manner. For deficiency state diseases, ceDNA vectors can be used to deliver transgenes to bring a normal gene into affected tissues for replacement therapy, as well, in some embodiments, to create animal models for the disease using antisense mutations. For unbalanced disease states, ceDNA vectors can be used to create a disease state in a model system, which could then be used in efforts to counteract the disease state. Thus the ceDNA vectors and methods disclosed herein permit the treatment of genetic diseases. As used herein, a disease state is treated by partially or wholly remedying the deficiency or imbalance that causes the disease or makes it more severe.

A. Host Cells:

In some embodiments, the ceDNA vector delivers the transgene into a subject host cell. In some embodiments, the subject host cell is a human host cell, including, for example blood cells, stem cells, hematopoietic cells, CD34⁺ cells, liver cells, cancer cells, vascular cells, muscle cells, pancreatic cells, neural cells, ocular or retinal cells, epithelial or endothelial cells, dendritic cells, fibroblasts, or any other cell of mammalian origin, including, without limitation, hepatic (i.e., liver) cells, lung cells, cardiac cells, pancreatic cells, intestinal cells, diaphragmatic cells, renal (i.e., kidney) cells, neural cells, blood cells, bone marrow cells, or any one or more selected tissues of a subject for which gene therapy is contemplated. In one aspect, the subject host cell is a human host cell.

The present disclosure also relates to recombinant host cells as mentioned above, including ceDNA vectors as described herein. Thus, one can use multiple host cells depending on the purpose as is obvious to the skilled artisan. A construct or ceDNA vector including donor sequence is introduced into a host cell so that the donor sequence is maintained as a chromosomal integrant as described earlier. The term host cell encompasses any progeny of a parent cell that is not identical to the parent cell due to mutations that occur during replication. The choice of a host cell will to a large extent depend upon the donor sequence and its source. The host cell may also be a eukaryote, such as a mammalian, insect, plant, or fungal cell. In one embodiment, the host cell is a human cell (e.g., a primary cell, a stem cell, or an immortalized cell line). In some embodiments, the host cell is gene edited for correction of a defective gene or to ablate expression of a gene. For Example, CRISPR/CAS can be used to edit the genome with one or more gRNA by either NHEJ or HDR repair, as well as other gene editing systems, e.g., ZFN or TALEs. The host cell can be any cell type, e.g., a somatic cell or a stem cell, an induced pluripotent stem cell, or a blood cell, e.g., T-cell or B-cell, or bone marrow cell. In certain embodiments, the host cell is an allogenic cell. For example, T-cell genome engineering is useful for cancer immunotherapies, disease modulation such as HIV therapy (e.g., receptor knock out, such as CXCR4 and CCR5) and immunodeficiency therapies. MHC receptors on B-cells can be targeted for immunotherapy. Genome edited bone marrow stem cells, e.g., CD34⁺ cells, or induced pluripotent stem cells can be transplanted back into a patient for expression of a therapeutic protein.

B. Exemplary Diseases to be Treated with a Gene Editing ceDNA

The ceDNA gene editing vectors are also useful for correcting a defective gene in the absence of donor DNA, e.g., one single guide RNA that targets a splice acceptor or splice donor can in a CRISPR/CAS ceDNA system correct a frameshift mutation in a defective gene and result in expression of functional protein. As a non-limiting example, DMD gene of Duchene Muscular Dystrophy has been corrected by exon skipping using a single guide RNA NHEJ, and by using multiple guide RNAs, for expression of a functional dystrophin, See e.g., US 2016/0201089, which is herein incorporated by reference in its entirety.

The ceDNA gene editing vectors are also useful for ablating gene expression. For example, in one embodiment a ceDNA vector can be used to cause a nonsense indel (e.g. an insertion or deletion of non-coding base pairs) to induce knockdown of a target gene, for example, by causing a frame-shift mutation. As a non-limiting example, expression of CXCR4 and CCR5, HIV receptors, have been successfully ablated in primary human T-cells by induction of either NHEJ or HDR pathways using CAS9 RNP and one or more guide RNA, See Schumann et al. (2015) Generation of knock in primary human cells using Cas9 ribonucleoproteins, PNAS 112(33): 10437-10442, herein incorporated by reference in its entirety. This system required only a single guide RNA and RNP (e.g., CAS9). CeDNA vectors can also be used to target the PD-1 locus in order to ablate expression. PD-1 expresses an immune checkpoint cell surface receptor on chronically active T cells that happens in malignancy. See Schumann et al. supra.

In some embodiments, the ceDNA gene editing vectors are used for correcting a defective gene by using a vector that targets the diseased gene. In one embodiment, the ceDNA vectors as described herein can be used to excise a desired region of DNA to correct a frameshift mutation, for example, to treat Duchenne muscular dystrophy or to remove mutated introns of LCA10 in the treatment of Leber Congenital Amaurosis. Non-limiting examples of diseases or disorders amenable to treatment by gene editing using ceDNA vectors, are listed in Tables A-C along with their and their associated genes of US patent publication 2014/0170753, which is herein incorporated by reference in its entirety. In alternative embodiments, the ceDNA vectors are used for insertion of an expression cassette for expression of a therapeutic protein or reporter protein in a safe harbor gene, e.g., in an inactive intron. In certain embodiments, a promoter-less cassette is inserted into the safe harbor gene. In such embodiments, a promoter-less cassette can take advantage of the safe harbor gene regulatory elements (promoters, enhancers, and signaling peptides), a non-limiting example of insertion at the safe harbor locus is insertion into to the albumin locus that is described in Blood (2015) 126 (15): 1777-1784, which is incorporated herein by reference in its entirety. Insertion into Albumin has the benefit of enabling secretion of the transgene into the blood (See e.g., Example 22). In addition, a genomic safe harbor site can be determined using techniques known in the art and described in, for example, Papapetrou, ER & Schambach, A. Molecular Therapy 24(4):678-684 (2016) or Sadelain et al. Nature Reviews Cancer 12:51-58 (2012), the contents of each of which are incorporated herein by reference in their entirety. It is specifically contemplated herein that safe harbor sites in an adeno associated virus (AAV) genome (e.g., AAVS1 safe harbor site) can be used with the methods and compositions described herein (see e.g., Oceguera-Yanez et al. Methods 101:43-55 (2016) or Tiyaboonchai, A et al. Stem Cell Res 12(3):630-7 (2014), the contents of each of which are incorporated by reference in their entirety). For example, the AAVS1 genomic safe harbor site can be used with the ceDNA vectors and compositions as described herein for the purposes of hematopoietic specific transgene expression and gene silencing in embryonic stem cells (e.g., human embryonic stem cells) or induced pluripotent stem cells (iPS cells). In addition, it is contemplated herein that synthetic or commercially available homology-directed repair donor templates for insertion into an AASV1 safe harbor site on chromosome 19 can be used with the ceDNA vectors or compositions as described herein. For example, homology-directed repair templates, and guide RNA, can be purchased commercially, for example, from System Biosciences, Palo Alto, Calif., and cloned into a ceDNA vector.

In some embodiments, the ceDNA vectors are used for knocking out or editing a gene in a T cell, e.g., to engineer the T cell for improved adoptive cell transfer and/or CAR-T therapies (see, e.g., Example 24). In some embodiments, the ceDNA vector can be a gene editing vector as described herein. In some embodiments, the ceDNA vector can comprise an endonuclease, a template nucleic acid sequence, or a combination of an endonuclease and template nucleic acid, as described elsewhere herein. Non-limiting examples of therapeutically relevant knock-outs and gene editing of T cells are described in PNAS (2015) 112(33):10437-10442, which is incorporated herein by reference in its entirety.

The gene editing ceDNA vector or a composition thereof can be used in the treatment of any hereditary disease. As a non-limiting example, the ceDNA vector or a composition thereof e.g. can be used in the treatment of transthyretin amyloidosis (ATTR), an orphan disease where the mutant protein misfolds and aggregates in nerves, the heart, the gastrointestinal system etc. It is contemplated herein that the disease can be treated by deletion of the mutant disease gene (mutTTR) using the gene editing systems described herein. Such treatments of hereditary diseases can halt disease progression and may enable regression of an established disease or reduction of at least one symptom of the disease by at least 10%.

In another embodiment, the ceDNA vector or a composition thereof can be used in the treatment of ornithine transcarbamylase deficiency (OTC deficiency), hyperammonaemia or other urea cycle disorders, which impair a neonate or infant's ability to detoxify ammonia. As with all diseases of inborn metabolism, it is contemplated herein that even a partial restoration of enzyme activity compared to wild-type controls (e.g., at least 20%, at least 30%, at least 40%, at least 50%, at least 60%, at least 70%, at least 80%, at least 90%, at least 95% or at least 99%) may be sufficient for reduction in at least one symptom OTC and/or an improvement in the quality of life for a subject having OTC deficiency. In one embodiment, a nucleic acid encoding OTC can be inserted behind the albumin endogenous promoter for in vivo protein replacement.

In another embodiment, the ceDNA vector or a composition thereof can be used in the treatment of phenylketonuria (PKU) by delivering a nucleic acid sequence encoding a phenylalanine hydroxylase enzyme to reduce buildup of dietary phenylalanine, which can be toxic to PKU sufferers. As with all diseases of inborn metabolism, it is contemplated herein that even a partial restoration of enzyme activity compared to wild-type controls (e.g., at least 20%, at least 30%, at least 40%, at least 50%, at least 60%, at least 70%, at least 80%, at least 90%, at least 95% or at least 99%) may be sufficient for reduction in at least one symptom of PKU and/or an improvement in the quality of life for a subject having PKU. In one embodiment, a nucleic acid encoding phenylalanine hydroxylase can be inserted behind the albumin endogenous promoter for in vivo protein replacement.

In another embodiment, the ceDNA vector or a composition thereof can be used in the treatment of glycogen storage disease (GSD) by delivering a nucleic acid sequence encoding an enzyme to correct aberrant glycogen synthesis or breakdown in subjects having GSD. Non-limiting examples of enzymes that can be corrected using the gene editing methods described herein include glycogen synthase, glucose-6-phosphatase, acid-alpha glucosidase, glycogen debranching enzyme, glycogen branching enzyme, muscle glycogen phosphorylase, liver glycogen phosphorylase, muscle phosphofructokinase, phosphorylase kinase, glucose transporter-2 (GLUT-2), aldolase A, beta-enolase, phosphoglucomutase-1 (PGM-1), and glycogenin-1. As with all diseases of inborn metabolism, it is contemplated herein that even a partial restoration of enzyme activity compared to wild-type controls (e.g., at least 20%, at least 30%, at least 40%, at least 50%, at least 60%, at least 70%, at least 80%, at least 90%, at least 95% or at least 99%) may be sufficient for reduction in at least one symptom of GSD and/or an improvement in the quality of life for a subject having GSD. In one embodiment, a nucleic acid encoding an enzyme to correct aberrant glycogen storage can be inserted behind the albumin endogenous promoter for in vivo protein replacement.

The ceDNA vectors described herein are also contemplated for use in the in vivo repair of Leber congenital amaurosis (LCA), polyglutamine diseases, including polyQ repeats, and alpha-1 antitrypsin deficiency (A1AT). LCA is a rare congenital eye disease resulting in blindness, which can be caused by a mutation in any one of the following genes: GUCY2D, RPE65, SPATA7, AIPL1, LCAS, RPGRIP1, CRX, CRB1, NMNAT1, CEP290, IMPDH1, RD3, RDH12, LRAT, TULP1, KCNJ13, GDF6 and/or PRPH2. It is contemplated herein that the gene editing methods and compositions as described herein can be adapted for delivery of one or more of the genes associated with LCA in order to correct an error in the gene(s) responsible for the symptoms of LCA. Polyglutamine diseases include, but are not limited to: dentatorubropallidoluysian atrophy, Huntington's disease, spinal and bulbar muscular atrophy, and spinocerebellar ataxia types 1, 2, 3 (also known as Machado-Joseph disease), 6, 7, and 17. It is specifically contemplated herein that the gene editing methods using ceDNA vectors can be used to repair DNA mutations resulting in trinucleotide repeat expansions (e.g., polyQ repeats), such as those associated with polyglutamine diseases. A1AT deficiency is a genetic disorder that causes defective production of alpha-1 antitrypsin, leading to decreased activity of the enzyme in the blood and lungs, which in turn can lead to emphysema or chronic obstructive pulmonary disease in affected subjects. Repair of A1AT deficiency is specifically contemplated herein using the ceDNA vectors or compositions thereof as outlined herein. It is contemplated herein that a nucleic acid encoding a desired protein for the treatment of LCA, polyglutamine diseases or A1AT deficiency can be inserted behind the albumin endogenous promoter for in vivo protein replacement.

In further embodiments, the compositions comprising a ceDNA vector as described herein can be used to edit a gene in a viral sequence, a pathogen sequence, a chromosomal sequence, a translocation junction (e.g., a translocation associated with cancer), a non-coding RNA gene or RNA sequence, a disease associated gene, among others.

Any nucleic acid or target gene of interest may be edited using the gene editing ceDNA vector as disclosed herein. Target nucleic acids and target genes include, but are not limited to nucleic acids encoding polypeptides, or non-coding nucleic acids (e.g., RNAi, miRs etc.) preferably therapeutic (e.g., for medical, diagnostic, or veterinary uses) or immunogenic (e.g., for vaccines) polypeptides. In certain embodiments, the target nucleic acids or target genes that are targeted by the gene editing ceDNA vectors as described herein encode one or more polypeptides, peptides, ribozymes, peptide nucleic acids, siRNAs, RNAis, antisense oligonucleotides, antisense polynucleotides, antibodies, antigen binding fragments, or any combination thereof.

In particular, a gene target for gene editing by the ceDNA vector disclosed herein can encode, for example, but is not limited to, protein(s), polypeptide(s), peptide(s), enzyme(s), antibodies, antigen binding fragments, as well as variants, and/or active fragments thereof, for use in the treatment, prophylaxis, and/or amelioration of one or more symptoms of a disease, dysfunction, injury, and/or disorder. In one aspect, the disease, dysfunction, trauma, injury and/or disorder is a human disease, dysfunction, trauma, injury, and/or disorder.

As noted herein, the gene target for gene editing using the ceDNA vector disclosed herein can encode a protein or peptide, or therapeutic nucleic acid sequence or therapeutic agent, including but not limited to one or more agonists, antagonists, anti-apoptosis factors, inhibitors, receptors, cytokines, cytotoxins, erythropoietic agents, glycoproteins, growth factors, growth factor receptors, hormones, hormone receptors, interferons, interleukins, interleukin receptors, nerve growth factors, neuroactive peptides, neuroactive peptide receptors, proteases, protease inhibitors, protein decarboxylases, protein kinases, protein kinase inhibitors, enzymes, receptor binding proteins, transport proteins or one or more inhibitors thereof, serotonin receptors, or one or more uptake inhibitors thereof, serpins, serpin receptors, tumor suppressors, diagnostic molecules, chemotherapeutic agents, cytotoxins, or any combination thereof.

C. Additional Diseases for Gene Editing:

In general, the ceDNA vector as disclosed herein can be used to deliver any transgene in accordance with the description above to treat, prevent, or ameliorate the symptoms associated with any disorder related to gene expression. Illustrative disease states include, but are not-limited to: cystic fibrosis (and other diseases of the lung), hemophilia A, hemophilia B, thalassemia, anemia and other blood disorders, AIDS, Alzheimer's disease, Parkinson's disease, Huntington's disease, amyotrophic lateral sclerosis, epilepsy, and other neurological disorders, cancer, diabetes mellitus, muscular dystrophies (e.g., Duchenne, Becker), Hurler's disease, adenosine deaminase deficiency, metabolic defects, retinal degenerative diseases (and other diseases of the eye), mitochondriopathies (e.g., Leber's hereditary optic neuropathy (LHON), Leigh syndrome, and subacute sclerosing encephalopathy), myopathies (e.g., facioscapulohumeral myopathy (FSHD) and cardiomyopathies), diseases of solid organs (e.g., brain, liver, kidney, heart), and the like. In some embodiments, the ceDNA vectors as disclosed herein can be advantageously used in the treatment of individuals with metabolic disorders (e.g., omithine transcarbamylase deficiency).

In some embodiments, the ceDNA vector described herein can be used to treat, ameliorate, and/or prevent a disease or disorder caused by mutation in a gene or gene product. Exemplary diseases or disorders that can be treated with a ceDNA vectors include, but are not limited to, metabolic diseases or disorders (e.g., Fabry disease, Gaucher disease, phenylketonuria (PKU), glycogen storage disease); urea cycle diseases or disorders (e.g., ornithine transcarbamylase (OTC) deficiency); lysosomal storage diseases or disorders (e.g., metachromatic leukodystrophy (MLD), mucopolysaccharidosis Type II (MPSII; Hunter syndrome)); liver diseases or disorders (e.g., progressive familial intrahepatic cholestasis (PFIC); blood diseases or disorders (e.g., hemophilia (A and B), thalassemia, and anemia); cancers and tumors, and genetic diseases or disorders (e.g., cystic fibrosis).

As still a further aspect, a ceDNA vector as disclosed herein may be employed to deliver a heterologous nucleotide sequence in situations in which it is desirable to regulate the level of transgene expression (e.g., transgenes encoding hormones or growth factors, as described herein).

Accordingly, in some embodiments, the ceDNA vector described herein can be used to correct an abnormal level and/or function of a gene product (e.g., an absence of, or a defect in, a protein) that results in the disease or disorder. The ceDNA vector can produce a functional protein and/or modify levels of the protein to alleviate or reduce symptoms resulting from, or confer benefit to, a particular disease or disorder caused by the absence or a defect in the protein. For example, treatment of OTC deficiency can be achieved by producing functional OTC enzyme; treatment of hemophilia A and B can be achieved by modifying levels of Factor VIII, Factor IX, and Factor X; treatment of PKU can be achieved by modifying levels of phenylalanine hydroxylase enzyme; treatment of Fabry or Gaucher disease can be achieved by producing functional alpha galactosidase or beta glucocerebrosidase, respectively; treatment of MLD or MPSII can be achieved by producing functional arylsulfatase A or iduronate-2-sulfatase, respectively; treatment of cystic fibrosis can be achieved by producing functional cystic fibrosis transmembrane conductance regulator; treatment of glycogen storage disease can be achieved by restoring functional G6Pase enzyme function; and treatment of PFIC can be achieved by producing functional ATP8B1, ABCB11, ABCB4, or TJP2 genes.

In alternative embodiments, the ceDNA vectors as disclosed herein can be used to provide an antisense nucleic acid to a cell in vitro or in vivo. For example, where the transgene is a RNAi molecule, expression of the antisense nucleic acid or RNAi in the target cell diminishes expression of a particular protein by the cell. Accordingly, transgenes which are RNAi molecules or antisense nucleic acids may be administered to decrease expression of a particular protein in a subject in need thereof. Antisense nucleic acids may also be administered to cells in vitro to regulate cell physiology, e.g., to optimize cell or tissue culture systems.

In some embodiments, exemplary transgenes encoded by the ceDNA vector include, but are not limited to: X, lysosomal enzymes (e.g., hexosaminidase A, associated with Tay-Sachs disease, or iduronate sulfatase, associated, with Hunter Syndrome/MPS II), erythropoietin, angiostatin, endostatin, superoxide dismutase, globin, leptin, catalase, tyrosine hydroxylase, as well as cytokines (e.g., a interferon, β-interferon, interferon-y, interleukin-2, interleukin-4, interleukin 12, granulocyte-macrophage colony stimulating factor, lymphotoxin, and the like), peptide growth factors and hormones (e.g., somatotropin, insulin, insulin-like growth factors 1 and 2, platelet derived growth factor (PDGF), epidermal growth factor (EGF), fibroblast growth factor (FGF), nerve growth factor (NGF), neurotrophic factor-3 and 4, brain-derived neurotrophic factor (BDNF), glial derived growth factor (GDNF), transforming growth factor-α and -β, and the like), receptors (e.g., tumor necrosis factor receptor). In some exemplary embodiments, the transgene encodes a monoclonal antibody specific for one or more desired targets. In some exemplary embodiments, more than one transgene is encoded by the ceDNA vector. In some exemplary embodiments, the transgene encodes a fusion protein comprising two different polypeptides of interest. In some embodiments, the transgene encodes an antibody, including a full-length antibody or antibody fragment, as defined herein. In some embodiments, the antibody is an antigen-binding domain or an immunoglobulin variable domain sequence, as that is defined herein. Other illustrative transgene sequences encode suicide gene products (thymidine kinase, cytosine deaminase, diphtheria toxin, cytochrome P450, deoxycytidine kinase, and tumor necrosis factor), proteins conferring resistance to a drug used in cancer therapy, and tumor suppressor gene products.

In a representative embodiment, the transgene expressed by the ceDNA vector can be used for the treatment of muscular dystrophy in a subject in need thereof, the method comprising: administering a treatment-, amelioration- or prevention-effective amount of ceDNA vector described herein, wherein the ceDNA vector comprises a heterologous nucleic acid encoding dystrophin, a mini-dystrophin, a micro-dystrophin, myostatin propeptide, follistatin, activin type II soluble receptor, IGF-1, anti-inflammatory polypeptides such as the Ikappa B dominant mutant, sarcospan, utrophin, a micro-dystrophin, laminin-α2, α-sarcoglycan, β-sarcoglycan, γ-sarcoglycan, δ-sarcoglycan, IGF-1, an antibody or antibody fragment against myostatin or myostatin propeptide, and/or RNAi against myostatin. In particular embodiments, the ceDNA vector can be administered to skeletal, diaphragm and/or cardiac muscle as described elsewhere herein.

In some embodiments, the ceDNA vector can be used to deliver a transgene to skeletal, cardiac or diaphragm muscle, for production of a polypeptide (e.g., an enzyme) or functional RNA (e.g., RNAi, microRNA, antisense RNA) that normally circulates in the blood or for systemic delivery to other tissues to treat, ameliorate, and/or prevent a disorder (e.g., a metabolic disorder, such as diabetes (e.g., insulin), hemophilia (e.g., VIII), a mucopolysaccharide disorder (e.g., Sly syndrome, Hurler Syndrome, Scheie Syndrome, Hurler-Scheie Syndrome, Hunter's Syndrome, Sanfilippo Syndrome A, B, C, D, Morquio Syndrome, Maroteaux-Lamy Syndrome, etc.) or a lysosomal storage disorder (such as Gaucher's disease [glucocerebrosidase], Pompe disease [lysosomal acid .alpha.-glucosidase] or Fabry disease [.alpha.-galactosidase A]) or a glycogen storage disorder (such as Pompe disease [lysosomal acid a glucosidase]). Other suitable proteins for treating, ameliorating, and/or preventing metabolic disorders are described above.

In other embodiments, the ceDNA vector as disclosed herein can be used to deliver a transgene in a method of treating, ameliorating, and/or preventing a metabolic disorder in a subject in need thereof. Illustrative metabolic disorders and transgenes encoding polypeptides are described herein. Optionally, the polypeptide is secreted (e.g., a polypeptide that is a secreted polypeptide in its native state or that has been engineered to be secreted, for example, by operable association with a secretory signal sequence as is known in the art).

Another aspect of the invention relates to a method of treating, ameliorating, and/or preventing congenital heart failure or PAD in a subject in need thereof, the method comprising administering a ceDNA vector as described herein to a mammalian subject, wherein the ceDNA vector comprises a transgene encoding, for example, a sarcoplasmic endoreticulum Ca²⁺-ATPase (SERCA2a), an angiogenic factor, phosphatase inhibitor I (I-1), RNAi against phospholamban; a phospholamban inhibitory or dominant-negative molecule such as phospholamban 516E, a zinc finger protein that regulates the phospholamban gene, β2-adrenergic receptor, .beta.2-adrenergic receptor kinase (BARK), PI3 kinase, calsarcan, a .beta.-adrenergic receptor kinase inhibitor (βARKct), inhibitor 1 of protein phosphatase 1, S100A1, parvalbumin, adenylyl cyclase type 6, a molecule that effects G-protein coupled receptor kinase type 2 knockdown such as a truncated constitutively active βARKct, Pim-1, PGC-1α, SOD-1, SOD-2, EC-SOD, kallikrein, HIF, thymosin-β4, mir-1, mir-133, mir-206 and/or mir-208.

The ceDNA vectors as disclosed herein can be administered to the lungs of a subject by any suitable means, optionally by administering an aerosol suspension of respirable particles comprising the ceDNA vectors, which the subject inhales. The respirable particles can be liquid or solid. Aerosols of liquid particles comprising the ceDNA vectors may be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the ceDNA vectors may likewise be produced with any solid particulate medicament aerosol generator, by techniques known in the pharmaceutical art.

In some embodiments, the ceDNA vectors can be administered to tissues of the CNS (e.g., brain, eye). In particular embodiments, the ceDNA vectors as disclosed herein may be administered to treat, ameliorate, or prevent diseases of the CNS, including genetic disorders, neurodegenerative disorders, psychiatric disorders and tumors. Illustrative diseases of the CNS include, but are not limited to Alzheimer's disease, Parkinson's disease, Huntington's disease, Canavan disease, Leigh's disease, Refsum disease, Tourette syndrome, primary lateral sclerosis, amyotrophic lateral sclerosis, progressive muscular atrophy, Pick's disease, muscular dystrophy, multiple sclerosis, myasthenia gravis, Binswanger's disease, trauma due to spinal cord or head injury, Tay Sachs disease, Lesch-Nyan disease, epilepsy, cerebral infarcts, psychiatric disorders including mood disorders (e.g., depression, bipolar affective disorder, persistent affective disorder, secondary mood disorder), schizophrenia, drug dependency (e.g., alcoholism and other substance dependencies), neuroses (e.g., anxiety, obsessional disorder, somatoform disorder, dissociative disorder, grief, post-partum depression), psychosis (e.g., hallucinations and delusions), dementia, paranoia, attention deficit disorder, psychosexual disorders, sleeping disorders, pain disorders, eating or weight disorders (e.g., obesity, cachexia, anorexia nervosa, and bulimia) and cancers and tumors (e.g., pituitary tumors) of the CNS.

Ocular disorders that may be treated, ameliorated, or prevented with the ceDNA vectors of the invention include ophthalmic disorders involving the retina, posterior tract, and optic nerve (e.g., retinitis pigmentosa, diabetic retinopathy and other retinal degenerative diseases, uveitis, age-related macular degeneration, glaucoma). Many ophthalmic diseases and disorders are associated with one or more of three types of indications: (1) angiogenesis, (2) inflammation, and (3) degeneration. In some embodiments, the ceDNA vector as disclosed herein can be employed to deliver anti-angiogenic factors; anti-inflammatory factors; factors that retard cell degeneration, promote cell sparing, or promote cell growth and combinations of the foregoing. Diabetic retinopathy, for example, is characterized by angiogenesis. Diabetic retinopathy can be treated by delivering one or more anti-angiogenic factors either intraocularly (e.g., in the vitreous) or periocularly (e.g., in the sub-Tenon's region). One or more neurotrophic factors may also be co-delivered, either intraocularly (e.g., intravitreally) or periocularly. Additional ocular diseases that may be treated, ameliorated, or prevented with the ceDNA vectors of the invention include geographic atrophy, vascular or “wet” macular degeneration, Stargardt disease, Leber Congenital Amaurosis (LCA), Usher syndrome, pseudoxanthoma elasticum (PXE), x-linked retinitis pigmentosa (XLRP), x-linked retinoschisis (XLRS), Choroideremia, Leber hereditary optic neuropathy (LHON), Archomatopsia, cone-rod dystrophy, Fuchs endothelial corneal dystrophy, diabetic macular edema and ocular cancer and tumors.

In some embodiments, inflammatory ocular diseases or disorders (e.g., uveitis) can be treated, ameliorated, or prevented by the ceDNA vectors of the invention. One or more anti-inflammatory factors can be expressed by intraocular (e.g., vitreous or anterior chamber) administration of the ceDNA vector as disclosed herein. In other embodiments, ocular diseases or disorders characterized by retinal degeneration (e.g., retinitis pigmentosa) can be treated, ameliorated, or prevented by the ceDNA vectors of the invention. intraocular (e.g., vitreal administration) of the ceDNA vector as disclosed herein encoding one or more neurotrophic factors can be used to treat such retinal degeneration-based diseases. In some embodiments, diseases or disorders that involve both angiogenesis and retinal degeneration (e.g., age-related macular degeneration) can be treated with the ceDNA vectors of the invention. Age-related macular degeneration can be treated by administering the ceDNA vector as disclosed herein encoding one or more neurotrophic factors intraocularly (e.g., vitreous) and/or one or more anti-angiogenic factors intraocularly or periocularly (e.g., in the sub-Tenon's region). Glaucoma is characterized by increased ocular pressure and loss of retinal ganglion cells. Treatments for glaucoma include administration of one or more neuroprotective agents that protect cells from excitotoxic damage using the ceDNA vector as disclosed herein. Accordingly, such agents include N-methyl-D-aspartate (NMDA) antagonists, cytokines, and neurotrophic factors, can be delivered intraocularly, optionally intravitreally using the ceDNA vector as disclosed herein.

In other embodiments, the ceDNA vector as disclosed herein may be used to treat seizures, e.g., to reduce the onset, incidence or severity of seizures. The efficacy of a therapeutic treatment for seizures can be assessed by behavioral (e.g., shaking, ticks of the eye or mouth) and/or electrographic means (most seizures have signature electrographic abnormalities). Thus, the ceDNA vector as disclosed herein can also be used to treat epilepsy, which is marked by multiple seizures over time. In one representative embodiment, somatostatin (or an active fragment thereof) is administered to the brain using the ceDNA vector as disclosed herein to treat a pituitary tumor. According to this embodiment, the ceDNA vector as disclosed herein encoding somatostatin (or an active fragment thereof) is administered by microinfusion into the pituitary. Likewise, such treatment can be used to treat acromegaly (abnormal growth hormone secretion from the pituitary). The nucleic acid (e.g., GenBank Accession No. J00306) and amino acid (e.g., GenBank Accession No. P01166; contains processed active peptides somatostatin-28 and somatostatin-14) sequences of somatostatins as are known in the art. In particular embodiments, the ceDNA vector can encode a transgene that comprises a secretory signal as described in U.S. Pat. No. 7,071,172.

Another aspect of the invention relates to the use of a ceDNA vector as described herein to produce antisense RNA, RNAi or other functional RNA (e.g., a ribozyme) for systemic delivery to a subject in vivo. Accordingly, in some embodiments, the ceDNA vector can comprise a transgene that encodes an antisense nucleic acid, a ribozyme (e.g., as described in U.S. Pat. No. 5,877,022), RNAs that affect spliceosome-mediated trans-splicing (see, Puttaraju et al., (1999) Nature Biotech. 17:246; U.S. Pat. Nos. 6,013,487; 6,083,702), interfering RNAs (RNAi) that mediate gene silencing (see, Sharp et al., (2000) Science 287:2431) or other non-translated RNAs, such as “guide” RNAs (Gorman et al., (1998) Proc. Nat. Acad. Sci. USA 95:4929; U.S. Pat. No. 5,869,248 to Yuan et al.), and the like.

In some embodiments, the ceDNA vector can further also comprise a transgene that encodes a reporter polypeptide (e.g., an enzyme such as Green Fluorescent Protein, or alkaline phosphatase). In some embodiments, a transgene that encodes a reporter protein useful for experimental or diagnostic purposes, is selected from any of: β-lactamase, β-galactosidase (LacZ), alkaline phosphatase, thymidine kinase, green fluorescent protein (GFP), chloramphenicol acetyltransferase (CAT), luciferase, and others well known in the art. In some aspects, ceDNA vectors comprising a transgene encoding a reporter polypeptide may be used for diagnostic purposes or as markers of the ceDNA vector's activity in the subject to which they are administered.

In some embodiments, the ceDNA vector can comprise a transgene or a heterologous nucleotide sequence that shares homology with, and recombines with a locus on the host chromosome. This approach may be utilized to correct a genetic defect in the host cell.

In some embodiments, the ceDNA vector can comprise a transgene that can be used to express an immunogenic polypeptide in a subject, e.g., for vaccination. The transgene may encode any immunogen of interest known in the art including, but not limited to, immunogens from human immunodeficiency virus, influenza virus, gag proteins, tumor antigens, cancer antigens, bacterial antigens, viral antigens, and the like.

D. Testing for Successful Gene Editing Using a Gene Editing ceDNA Vector

Assays well known in the art can be used to test the efficiency of gene editing by ceDNA in both in vitro and in vivo models. Knock-in or knock-out of a desired transgene by ceDNA can be assessed by one skilled in the art by measuring mRNA and protein levels of the desired transgene (e.g., reverse transcription PCR, western blot analysis, and enzyme-linked immunosorbent assay (ELISA)). Nucleic acid alterations by ceDNA (e.g., point mutations, or deletion of DNA regions) can be assessed by deep sequencing of genomic target DNA. In one embodiment, ceDNA comprises a reporter protein that can be used to assess the expression of the desired transgene, for example by examining the expression of the reporter protein by fluorescence microscopy or a luminescence plate reader. For in vivo applications, protein function assays can be used to test the functionality of a given gene and/or gene product to determine if gene editing has successfully occurred. For example, it is envisioned that a point mutation in the cystic fibrosis transmembrane conductance regulator gene (CFTR) inhibits the capacity of CFTR to move anions (e.g., Cl⁻) through the anion channel, can be corrected by ceDNA's gene editing capacity. Following administration of ceDNA, one skilled in the art can assess the capacity for anions to move through the anion channel to determine if the point mutation of CFTR has been corrected. One skilled will be able to determine the best test for measuring functionality of a protein in vitro or in vivo.

It is contemplated herein that the effects of gene editing in a cell or subject can last for at least 1 month, at least 2 months, at least 3 months, at least four months, at least 5 months, at least six months, at least 10 months, at least 12 months, at least 18 months, at least 2 years, at least 5 years, at least 10 years, at least 20 years, or can be permanent.

In some embodiments, a transgene in the expression cassette, expression construct, or ceDNA vector described herein can be codon optimized for the host cell. As used herein, the term “codon optimized” or “codon optimization” refers to the process of modifying a nucleic acid sequence for enhanced expression in the cells of the vertebrate of interest, e.g., mouse or human (e.g., humanized), by replacing at least one, more than one, or a significant number of codons of the native sequence (e.g., a prokaryotic sequence) with codons that are more frequently or most frequently used in the genes of that vertebrate. Various species exhibit particular bias for certain codons of a particular amino acid. Typically, codon optimization does not alter the amino acid sequence of the original translated protein. Optimized codons can be determined using e.g., Aptagen's Gene Forge® codon optimization and custom gene synthesis platform (Aptagen, Inc.) or another publicly available database.

### XIII. Administration

In particular embodiments, more than one administration (e.g., two, three, four or more administrations) may be employed to achieve the desired level of gene expression over a period of various intervals, e.g., daily, weekly, monthly, yearly, etc.

Exemplary modes of administration of the ceDNA vector disclosed herein includes oral, rectal, transmucosal, intranasal, inhalation (e.g., via an aerosol), buccal (e.g., sublingual), vaginal, intrathecal, intraocular, transdermal, intraendothelial, in utero (or in ovo), parenteral (e.g., intravenous, subcutaneous, intradermal, intracranial, intramuscular [including administration to skeletal, diaphragm and/or cardiac muscle], intrapleural, intracerebral, and intraarticular), topical (e.g., to both skin and mucosal surfaces, including airway surfaces, and transdermal administration), intralymphatic, and the like, as well as direct tissue or organ injection (e.g., to liver, eye, skeletal muscle, cardiac muscle, diaphragm muscle or brain).

Administration of the ceDNA vector can be to any site in a subject, including, without limitation, a site selected from the group consisting of the brain, a skeletal muscle, a smooth muscle, the heart, the diaphragm, the airway epithelium, the liver, the kidney, the spleen, the pancreas, the skin, and the eye. Administration of the ceDNA vector can also be to a tumor (e.g., in or near a tumor or a lymph node). The most suitable route in any given case will depend on the nature and severity of the condition being treated, ameliorated, and/or prevented and on the nature of the particular ceDNA vector that is being used. Additionally, ceDNA permits one to administer more than one transgene in a single vector, or multiple ceDNA vectors (e.g. a ceDNA cocktail)

Administration of the ceDNA vector disclosed herein to skeletal muscle according to the present invention includes but is not limited to administration to skeletal muscle in the limbs (e.g., upper arm, lower arm, upper leg, and/or lower leg), back, neck, head (e.g., tongue), thorax, abdomen, pelvis/perineum, and/or digits. The ceDNA as disclosed herein vector can be delivered to skeletal muscle by intravenous administration, intra-arterial administration, intraperitoneal administration, limb perfusion, (optionally, isolated limb perfusion of a leg and/or arm; see, e.g. Arruda et al., (2005) Blood 105: 3458-3464), and/or direct intramuscular injection. In particular embodiments, the ceDNA vector as disclosed herein is administered to a limb (arm and/or leg) of a subject (e.g., a subject with muscular dystrophy such as DMD) by limb perfusion, optionally isolated limb perfusion (e.g., by intravenous or intra-articular administration. In certain embodiments, the ceDNA vector as disclosed herein can be administered without employing “hydrodynamic” techniques.

Administration of the ceDNA vector as disclosed herein to cardiac muscle includes administration to the left atrium, right atrium, left ventricle, right ventricle and/or septum. The ceDNA vector as described herein can be delivered to cardiac muscle by intravenous administration, intra-arterial administration such as intra-aortic administration, direct cardiac injection (e.g., into left atrium, right atrium, left ventricle, right ventricle), and/or coronary artery perfusion. Administration to diaphragm muscle can be by any suitable method including intravenous administration, intra-arterial administration, and/or intra-peritoneal administration. Administration to smooth muscle can be by any suitable method including intravenous administration, intra-arterial administration, and/or intra-peritoneal administration. In one embodiment, administration can be to endothelial cells present in, near, and/or on smooth muscle.

In some embodiments, a ceDNA vector according to the present invention is administered to skeletal muscle, diaphragm muscle and/or cardiac muscle (e.g., to treat, ameliorate and/or prevent muscular dystrophy or heart disease (e.g., PAD or congestive heart failure).

A. Ex Vivo Treatment

In some embodiments, cells are removed from a subject, a ceDNA vector is introduced therein, and the cells are then replaced back into the subject. Methods of removing cells from subject for treatment ex vivo, followed by introduction back into the subject are known in the art (see, e.g., U.S. Pat. No. 5,399,346; the disclosure of which is incorporated herein in its entirety). Alternatively, a ceDNA vector is introduced into cells from another subject, into cultured cells, or into cells from any other suitable source, and the cells are administered to a subject in need thereof.

Cells transduced with a ceDNA vector are preferably administered to the subject in a “therapeutically-effective amount” in combination with a pharmaceutical carrier. Those skilled in the art will appreciate that the therapeutic effects need not be complete or curative, as long as some benefit is provided to the subject.

In some embodiments, the ceDNA vector can encode a transgene (sometimes called a heterologous nucleotide sequence) that is any polypeptide that is desirably produced in a cell in vitro, ex vivo, or in vivo. For example, in contrast to the use of the ceDNA vectors in a method of treatment as discussed herein, in some embodiments the ceDNA vectors may be introduced into cultured cells and the expressed gene product isolated therefrom, e.g., for the production of antigens or vaccines.

The ceDNA vectors can be used in both veterinary and medical applications. Suitable subjects for ex vivo gene delivery methods as described above include both avians (e.g., chickens, ducks, geese, quail, turkeys and pheasants) and mammals (e.g., humans, bovines, ovines, caprines, equines, felines, canines, and lagomorphs), with mammals being preferred. Human subjects are most preferred. Human subjects include neonates, infants, juveniles, and adults.

One aspect of the technology described herein relates to a method of delivering a transgene to a cell. Typically, for in vitro methods, the ceDNA vector may be introduced into the cell using the methods as disclosed herein, as well as other methods known in the art. ceDNA vectors disclosed herein are preferably administered to the cell in a biologically-effective amount. If the ceDNA vector is administered to a cell in vivo (e.g., to a subject), a biologically-effective amount of the ceDNA vector is an amount that is sufficient to result in transduction and expression of the transgene in a target cell.

B. Dose Ranges

In vivo and/or in vitro assays can optionally be employed to help identify optimal dosage ranges for use. The precise dose to be employed in the formulation will also depend on the route of administration, and the seriousness of the condition, and should be decided according to the judgment of the person of ordinary skill in the art and each subject's circumstances. Effective doses can be extrapolated from dose-response curves derived from in vitro or animal model test systems.

A ceDNA vector is administered in sufficient amounts to transfect the cells of a desired tissue and to provide sufficient levels of gene transfer and expression without undue adverse effects. Conventional and pharmaceutically acceptable routes of administration include, but are not limited to, those described above in the “Administration” section, such as direct delivery to the selected organ (e.g., intraportal delivery to the liver), oral, inhalation (including intranasal and intratracheal delivery), intraocular, intravenous, intramuscular, subcutaneous, intradermal, intratumoral, and other parental routes of administration. Routes of administration can be combined, if desired.

The dose of the amount of a ceDNA vector required to achieve a particular “therapeutic effect,” will vary based on several factors including, but not limited to: the route of nucleic acid administration, the level of gene or RNA expression required to achieve a therapeutic effect, the specific disease or disorder being treated, and the stability of the gene(s), RNA product(s), or resulting expressed protein(s). One of skill in the art can readily determine a ceDNA vector dose range to treat a patient having a particular disease or disorder based on the aforementioned factors, as well as other factors that are well known in the art.

Dosage regime can be adjusted to provide the optimum therapeutic response. For example, the oligonucleotide can be repeatedly administered, e.g., several doses can be administered daily or the dose can be proportionally reduced as indicated by the exigencies of the therapeutic situation. One of ordinary skill in the art will readily be able to determine appropriate doses and schedules of administration of the subject oligonucleotides, whether the oligonucleotides are to be administered to cells or to subjects.

A “therapeutically effective dose” will fall in a relatively broad range that can be determined through clinical trials and will depend on the particular application (neural cells will require very small amounts, while systemic injection would require large amounts). For example, for direct in vivo injection into skeletal or cardiac muscle of a human subject, a therapeutically effective dose will be on the order of from about 1 μg to 100 g of the ceDNA vector. If exosomes or microparticles are used to deliver the ceDNA vector, then a therapeutically effective dose can be determined experimentally, but is expected to deliver from 1 μg to about 100 g of vector. Moreover, a therapeutically effective dose is an amount ceDNA vector that expresses a sufficient amount of the gene editing molecule to have an effect on editing the target gene that results in a reduction in one or more symptoms of the disease, but does not result in gene editing of off-target genes.

Formulation of pharmaceutically-acceptable excipients and carrier solutions is well-known to those of skill in the art, as is the development of suitable dosing and treatment regimens for using the particular compositions described herein in a variety of treatment regimens.

For in vitro transfection, an effective amount of a ceDNA vector to be delivered to cells (1×10⁶ cells) will be on the order of 0.1 to 100 μg ceDNA vector, preferably 1 to 20 μg, and more preferably 1 to 15 μg or 8 to 10 μg. Larger ceDNA vectors will require higher doses. If exosomes or microparticles are used, an effective in vitro dose can be determined experimentally but would be intended to deliver generally the same amount of the ceDNA vector.

Treatment can involve administration of a single dose or multiple doses. In some embodiments, more than one dose can be administered to a subject; in fact multiple doses can be administered as needed, because the ceDNA vector elicits does not elicit an anti-capsid host immune response due to the absence of a viral capsid. As such, one of skill in the art can readily determine an appropriate number of doses. The number of doses administered can, for example, be on the order of 1-100, preferably 2-20 doses.

Without wishing to be bound by any particular theory, the lack of typical anti-viral immune response elicited by administration of a ceDNA vector as described by the disclosure (i.e., the absence of capsid components) allows the ceDNA vector to be administered to a host on multiple occasions. In some embodiments, the number of occasions in which a heterologous nucleic acid is delivered to a subject is in a range of 2 to 10 times (e.g., 2, 3, 4, 5, 6, 7, 8, 9, or 10 times). In some embodiments, a ceDNA vector is delivered to a subject more than 10 times.

In some embodiments, a dose of a ceDNA vector is administered to a subject no more than once per calendar day (e.g., a 24-hour period). In some embodiments, a dose of a ceDNA vector is administered to a subject no more than once per 2, 3, 4, 5, 6, or 7 calendar days. In some embodiments, a dose of a ceDNA vector is administered to a subject no more than once per calendar week (e.g., 7 calendar days). In some embodiments, a dose of a ceDNA vector is administered to a subject no more than bi-weekly (e.g., once in a two calendar week period). In some embodiments, a dose of a ceDNA vector is administered to a subject no more than once per calendar month (e.g., once in 30 calendar days). In some embodiments, a dose of a ceDNA vector is administered to a subject no more than once per six calendar months. In some embodiments, a dose of a ceDNA vector is administered to a subject no more than once per calendar year (e.g., 365 days or 366 days in a leap year).

C. Unit Dosage Forms

In some embodiments, the pharmaceutical compositions can conveniently be presented in unit dosage form. A unit dosage form will typically be adapted to one or more specific routes of administration of the pharmaceutical composition. In some embodiments, the unit dosage form is adapted for administration by inhalation. In some embodiments, the unit dosage form is adapted for administration by a vaporizer. In some embodiments, the unit dosage form is adapted for administration by a nebulizer. In some embodiments, the unit dosage form is adapted for administration by an aerosolizer. In some embodiments, the unit dosage form is adapted for oral administration, for buccal administration, or for sublingual administration. In some embodiments, the unit dosage form is adapted for intravenous, intramuscular, or subcutaneous administration. In some embodiments, the unit dosage form is adapted for intrathecal or intracerebroventricular administration. In some embodiments, the pharmaceutical composition is formulated for topical administration. The amount of active ingredient which can be combined with a carrier material to produce a single dosage form will generally be that amount of the compound which produces a therapeutic effect.

### XIV. Various Applications

The compositions and ceDNA vectors provided herein can be used to deliver a gene editing molecule for various purposes as described above. In some embodiments, the gene editing molecule targets a target gene, e.g., a protein or functional RNA, that is to be edited for research purposes, e.g., to create a somatic transgenic animal model harboring one or more mutations or a corrected gene sequence, e.g., to study the function of the target gene. In another example, the gene editing molecule is used to gene edit a target gene that encodes a protein or functional RNA to create an animal model of disease.

In some embodiments, the target gene of the gene editing molecule encodes one or more peptides, polypeptides, or proteins, which are useful for the treatment, amelioration, or prevention of disease states in a mammalian subject. The gene editing molecule can be transferred (e.g., expressed in) via the ceDNA vector, to a patient in a sufficient amount to treat a disease associated with an abnormal gene sequence, which can result in any one or more of the following: reduced expression, lack of expression or dysfunction of the target gene.

In some embodiments, the ceDNA vectors are envisioned for use in diagnostic and screening methods, whereby a gene editing molecule is transiently or stably expressed in a cell culture system, or alternatively, a transgenic animal model.

Another aspect of the technology described herein provides a method of transducing a population of mammalian cells. In an overall and general sense, the method includes at least the step of introducing into one or more cells of the population, a composition that comprises an effective amount of one or more of the ceDNA disclosed herein.

Additionally, the present invention provides compositions, as well as therapeutic and/or diagnostic kits that include one or more of the disclosed ceDNA vectors or ceDNA compositions, formulated with one or more additional ingredients, or prepared with one or more instructions for their use.

A cell to be administered the ceDNA vector as disclosed herein may be of any type, including but not limited to neural cells (including cells of the peripheral and central nervous systems, in particular, brain cells), lung cells, retinal cells, epithelial cells (e.g., gut and respiratory epithelial cells), muscle cells, dendritic cells, pancreatic cells (including islet cells), hepatic cells, myocardial cells, bone cells (e.g., bone marrow stem cells), hematopoietic stem cells, spleen cells, keratinocytes, fibroblasts, endothelial cells, prostate cells, germ cells, and the like. Alternatively, the cell may be any progenitor cell. As a further alternative, the cell can be a stem cell (e.g., neural stem cell, liver stem cell). As still a further alternative, the cell may be a cancer or tumor cell. Moreover, the cells can be from any species of origin, as indicated above.

In some embodiments, the present application may be defined in any of the following paragraphs:

1. A ceDNA vector comprising: (i) at least one altered AAV inverted terminal repeat (ITR); and (ii) a first nucleotide sequence comprising a 5′ homology arm, a donor sequence, and a 3′ homology arm, wherein at least the donor sequence has gene editing functionality.
2. The ceDNA vector of paragraph 1, wherein the first nucleotide sequence further comprises a second nucleotide sequence upstream the first nucleotide sequence, wherein the second nucleotide sequence comprises a gene regulatory sequence, and a nucleotide sequence encoding a nuclease, wherein the gene regulatory sequence is operably linked to the nucleotide sequence encoding the nuclease.
3. The ceDNA vector of any of paragraphs 1-2, wherein the nuclease is a sequence-specific nuclease.
4. The ceDNA vector of any of paragraphs 1-3, wherein the sequence-specific nuclease is an RNA-guided nuclease, zinc finger nuclease (ZFN), or a transcription activator-like effector nuclease (TALEN).
5. The ceDNA vector of any of paragraphs 1-4, wherein the RNA-guided nuclease is Cas or Cas9.
6. The ceDNA vector of any of paragraphs 1-5, wherein the regulatory sequence comprises an enhancer and a promoter, wherein the second nucleic acid sequence comprises an intron sequence upstream the nucleotide sequence encoding a nuclease, wherein the intron comprises a nuclease cleavage site, and wherein the promoter is operably linked to the nucleotide sequence encoding the nuclease.
7. The ceDNA vector of any of paragraphs 1-6, further comprising a third nucleotide sequence comprising a nucleotide sequence encoding a guide sequence and/or activator RNA sequence.
8. The ceDNA vector of any of paragraphs 1-7, wherein the third nucleotide sequence further comprises a promoter operably linked to the nucleotide sequence encoding the guide sequence and/or activator RNA sequence.
9. The ceDNA vector of any of paragraphs 1-8, wherein a poly-A site is upstream and proximate a said homology arm.
10. The ceDNA vector of any of paragraphs 1-9, wherein the donor sequence is foreign to the 5′ homology arm or 3′ homology arm.
11. The ceDNA vector of any of paragraphs 1-10, wherein the 5′ homology arm is homologous to a nucleotide sequence upstream of a nuclease cleavage site on a chromosome.
12. The ceDNA vector of any of paragraphs 1-11, wherein the 3′ homology arm is homologous to a nucleotide sequence downstream of a nuclease cleavage site on a chromosome.
13. The ceDNA vector of any of paragraphs 1-12, wherein the 5′ homology arm or the 3′ homology arm are proximal to the at least one altered ITR.
14. The ceDNA vector of any of paragraphs 1-13, wherein the 5′ homology arm and the 3′ homology arm are about 250 to 2000 bp.
15. The ceDNA vector of any of paragraphs 1-14, wherein the nucleotide sequence encoding a nuclease is cDNA.
16. The ceDNA vector of any of paragraphs 1-15, wherein the promoter is a CAG promoter.
17. The ceDNA vector of any of paragraphs 1-17, wherein the promoter is Pol III, U6, or H1.
18. A method of inserting a donor sequence at a predetermined insertion site on a chromosome in a host cell, comprising: introducing into the host cell a ceDNA vector having at least one altered ITR, wherein the ceDNA vector comprises a nucleotide sequence comprising a 5′ homology arm, a donor sequence, and a 3′ homology arm, wherein the donor sequence is inserted into the chromosome at or adjacent to the insertion site through homologous recombination.
19. The method of paragraph 18, further comprising introducing into the cell a nucleotide sequence encoding a guide RNA (gRNA) recognizing the insertion site.
20. The method of paragraph 18 or 19, further comprising introducing into the cell a nucleotide sequence encoding a sequence-specific nuclease that cleaves the chromosome at or adjacent to the insertion site.
21. The method of any of paragraphs 18-20, wherein the sequence-specific nuclease is an RNA-guided nuclease, zinc finger nuclease (ZFN), or a transcription activator-like effector nuclease (TALEN).
22. The method of any of paragraphs 18-21, wherein the RNA-guided nuclease is Cas or Cas9.
23. The method of any of paragraphs 18-22, wherein the step of introducing is capsid free.
24. The method of any of paragraphs 18-23, wherein the 5′ homology arm is homologous to a sequence upstream of the nuclease cleavage site on the chromosome.
25. The method of any of paragraphs 18-24, wherein the 3′ homology arm is homologous to a sequence downstream of the nuclease cleavage site on the chromosome.
26. The method of any of paragraphs 18, wherein the 5′ homology arm or the 3′ homology arm are proximal to the altered ITR.
27. The method of any of paragraphs 18-26, wherein the 5′ homology arm and the 3′ homology arm are at least about 50-2000 base pairs.
28. The method of any of paragraphs 18-27, wherein the nucleotide sequence further comprises a 5′ flanking sequence upstream of the 5′ homology arm and a 3′ flanking sequence downstream of the 3′ homology arm.
29. A method of generating a genetically modified animal comprising a donor sequence inserted at a predetermined insertion site on the chromosome of the animal, comprising a) generating a cell with the donor sequence inserted at the predetermined insertion site on the chromosome according to paragraph 18; and b) introducing the cell generated by a) into a carrier animal to produce the genetically modified animal.
30. The method of paragraphs 29, wherein the cell is a zygote or a pluripotent stem cell.
31. A genetically modified animal generated by the method of paragraph 29 or 30.
32. A kit for inserting a donor sequence at an insertion site on a chromosome in a cell, comprising: (a) a first ceDNA vector comprising: (i) at least one altered AAV inverted terminal repeat (ITR); and (ii) a first nucleotide sequence comprising a 5′ homology arm, a donor sequence, and a 3′ homology arm, wherein the donor sequence has gene editing functionality; and (b) a second ceDNA vector comprising: (i) at least one altered AAV inverted terminal repeat (ITR); and (ii) a nucleotide sequence encoding a nuclease, wherein the 5′ homology arm is homologous to a sequence upstream of a nuclease cleavage site on the chromosome and wherein the 3′ homology arm is homologous to a sequence downstream of the nuclease cleavage site on the chromosome; and wherein the 5′ homology arm or the 3′ homology arm are proximal to the an altered ITR.
33. A method of inserting a donor sequence at a predetermined insertion site on a chromosome in a host cell, comprising: (a) introducing into the host cell a first ceDNA vector having at least one altered ITR, wherein the ceDNA vector comprises a first linear nucleic acid comprising a 5′ homology arm, a donor sequence, and a 3′ homology arm; and (b) introducing into the host cell a second ceDNA vector having at least one altered ITR, wherein the second ceDNA vector comprises a second linear nucleic acid comprising a nucleotide sequence encoding a sequence-specific nuclease that cleaves the chromosome at or adjacent to the insertion site, wherein the donor sequence is inserted into the chromosome at or adjacent to the insertion site through homologous recombination.
34. The method of any of paragraphs 18-33, wherein the second ceDNA vector further comprises a third nucleotide sequence encoding a guide sequence recognizing the insertion site.
35. The ceDNA vector of any of paragraphs 1-17, further comprising at least one of a transgene enhancement element, and a poly-A cite down-stream and proximate the 3′ homology arm.
36. The ceDNA vector of any of paragraphs 1-17 or 35, further comprising an alternative nuclease target sequence proximate to the altered ITR.
37. The ceDNA vector of any of paragraphs 1-17 or 35-36, further comprising a 2A and selection marker site upstream and proximate to the 3′ homology arm.
38. A ceDNA nucleic acid vector composition comprising: flanking terminal repeats (TR); and at least one gene editing nucleic acid sequence, wherein the vector is a linear close-ended duplex DNA.
39. The composition of paragraph 38, wherein the terminal repeats are inverted TRs (ITRs).
40. The composition of paragraph 38 or 39, wherein at least one of the terminal repeats is modified.
41. The composition of any of paragraphs 38-40, wherein the vector is single stranded circular DNA under nucleic acid denaturing conditions.
42. The composition of any of paragraphs 38-41, wherein the gene editing nucleic acid sequence encodes gene editing molecule selected from the group consisting of: a sequence specific nuclease, one or more guide RNA, CRISPR/Cas, a ribonucleoprotein (RNP), or deactivated CAS for CRISPRi or CRISPRa systems, or any combination thereof.
43. The composition of any of paragraphs 38-42, wherein the sequence-specific nuclease comprises: a TAL-nuclease, a zinc-finger nuclease (ZFN), a meganuclease, a megaTAL, or an RNA guided endonuclease (e.g., CAS9, cpfl, dCAS9, nCAS9).
44. The composition of any of paragraphs 38-43, further comprising at least two modified ITRs.
45. The composition of any of paragraphs 38-44, further comprising a nucleic acid of interest.
46. The composition of any of paragraphs 38-45, wherein the gene editing nucleic acid sequence is a homology-directed repair template.
47. The composition of any of paragraphs 38-46, wherein the homology-directed repair template comprises a 5′ homology arm, a donor sequence, and a 3′ homology arm.
48. The composition of any of paragraphs 38-47, further comprising a nucleic acid sequence that encodes an endonuclease, wherein the endonuclease cleaves or nicks at a specific endonuclease site on DNA of a target gene or a target site on the ceDNA vector.
49. The composition of any of paragraphs 38-48, wherein the 5′ homology arm is homologous to a nucleotide sequence upstream of the DNA endonuclease site on a chromosome.
50. The composition of any of paragraphs 38-49, wherein the 3′ homology arm is homologous to a nucleotide sequence downstream of the DNA endonuclease site.
51. The composition of any of paragraphs 38-40, wherein the homology arms are each about 250 to 2000 bp.
52. The composition of any of paragraphs 38-52, wherein the DNA endonuclease comprises: a TAL-nuclease, a zinc-finger nuclease (ZFN), or an RNA guided endonuclease (e.g., Cas9 or Cpf1).
53. The composition of any of paragraphs 38-52, wherein the RNA guided endonuclease comprises a Cas enzyme.
54. The composition of any of paragraphs 38-53, wherein the Cas enzyme is Cas9.
55. The composition of any of paragraphs 38-53, wherein the Cas enzyme is nicking Cas9 (nCas9).
56. The composition of any of paragraphs 38-55, wherein the nCas9 comprises a mutation in the HNH or RuVc domain of Cas.
57. The composition of any of paragraphs 38-53, wherein the Cas enzyme is deactivated Cas nuclease (dCas) that complexes with a gRNA that targets a promoter region of a target gene.
58. The composition of any of paragraphs 38-57, further comprising a KRAB effector domain.
59. The composition of any of paragraphs 38-57, wherein the dCas is fused to a heterologous transcriptional activation domain that can be directed to a promoter region.
60. The composition of any of paragraphs 38-59, wherein the dCas fusion is directed to a promoter region of a target gene by a guide RNA that recruits additional transactivation domains to upregulate expression of the target gene.
61. The composition of any of paragraphs 38-57, wherein the dCas is S. pyogenes dCas9.
62. The composition of any of paragraphs 38-61, wherein the guide RNA sequence targets the proximity of the promoter of a target gene and CRISPR silences the target gene (CRISPRi system).
63. The composition of any of paragraphs 38-61, wherein the guide RNA sequence targets the transcriptional start site of a target gene and activates the target gene (CRISPRa system).
64. The composition of any of paragraphs 38-63, further comprising a nucleic acid encoding at least one guide RNA (gRNA) for a RNA-guided DNA endonuclease.
65. The composition of any of paragraphs 38-64, wherein the guide RNA (sgRNA) targets a splice acceptor or splice donor site of a defective gene to effect non-homologous end joining (NHEJ) and correction of the defective gene.
66. The composition of any of paragraphs 38-65, wherein the vector encodes multiple copies of one guide RNA sequence.
67. The composition of any of paragraphs 38-66, further comprising a regulatory sequence operably linked to the nucleic acid sequence encoding the nuclease.
68. The composition of any of paragraphs 38-67, wherein the regulatory sequence comprises an enhancer and/or a promoter.
69. The composition of any of paragraphs 38-68, wherein a promoter is operably linked to the nucleic acid sequence encoding the DNA endonuclease, wherein the nucleic acid sequence encoding the DNA endonuclease further comprises an intron sequence upstream of the endonuclease sequence, and wherein the intron comprises a nuclease cleavage site.
70. The composition of any of paragraphs 38-69, wherein a poly-A-site is upstream and proximate to the 5′ homology arm.
71. The composition of any of paragraphs 47***, wherein the donor sequence is foreign to the 5′ homology arm or the 3′ homology arm.
72. The composition of any of paragraphs 47, wherein the 5′ homology arm or the 3′ homology arm are proximal to the at least one modified ITR.
73. The composition of any of paragraphs 48, wherein the nucleotide sequence encoding a nuclease is cDNA.
74. The composition of any of paragraphs 68, wherein the promoter is a CAG promoter.
75. The composition of any of paragraphs 68, wherein the promoter is Pol III, U6, or H1.
76. A cell comprising a vector of any of paragraphs 38-75.
77. A composition comprising: a vector of any of paragraphs 38-75 and a lipid.
78. A kit comprising a vector of any of any of paragraphs 38-75, or a cell of paragraph 76.
79. A method for genome editing comprising: contacting a cell with a gene editing system, wherein one or more components of the gene editing system are delivered to the cell by contacting the cell with a close-ended DNA (ceDNA) nucleic acid vector composition, wherein the ceDNA nucleic acid vector composition is a linear close-ended duplex DNA comprising flanking terminal repeat (TR) sequences and optionally at least one gene editing nucleic acid sequence having a region complementary to at least one target gene.
80. The method of paragraph 79, wherein the terminal repeats are inverted TRs (ITRs).
81. The method of paragraph 79 or 80, wherein the ITR is a modified ITR.
82. The method of any of paragraphs 79-81, wherein the gene editing system is selected from the group consisting of: a TALEN system, a zinc-finger endonuclease (ZFN) system, a CRISPR/Cas system, and a meganuclease system.
83. The method of any of paragraphs 79-82, wherein the at least one gene editing nucleic acid sequence encodes a gene editing molecule selected from the group consisting of: an RNA guided nuclease, a guide RNA, a TALEN, and a zinc-finger endonuclease (ZFN).
84. The method of any of paragraphs 79-83, wherein a single ceDNA vector comprises all components of the gene editing system.
85. The method of any of paragraphs 79-84, wherein the step of contacting the cell further comprises administering a transfection reagent or lipid reagent in combination with the gene editing system.
86. The method of any of paragraphs 79-85, wherein the gene editing system further comprises a transfection reagent or liposome reagent.
87. The method of any of paragraphs 79-86, wherein the ceDNA nucleic acid vector composition is any one of paragraphs 1-77.
88. The method of any of paragraphs 79-87, wherein the expression of the target gene is altered.
89. The method of any of paragraphs 79-88, wherein the cell contacted is a eukaryotic cell.
90. The method of any of paragraphs 79-88, wherein the Cas protein is codon optimized for expression in the eukaryotic cell.
91. A method of genome editing comprising administering to a cell an effective amount of a ceDNA composition of any one of paragraphs 1-77, under conditions suitable and for a time sufficient to edit a target gene.
92. The method of any of paragraphs 79-91, wherein the target gene is gene targeted using one or more guide RNA sequences and edited by homology directed repair (HDR) in the presence of a HDR donor template.
93. The method of any of paragraphs 79-91, wherein the target gene is targeted using one guide RNA sequence and the target gene is edited by non-homologous end joining (NHEJ).
94. The method of any one of paragraphs 79-93, wherein the method is performed in vivo to correct a single nucleotide polymorphism (SNP) associated with a disease.
95. The method of any of paragraphs 94, wherein the disease comprises sickle cell anemia, hereditary hemochromatosis or cancer hereditary blindness.
96. The method of any of paragraphs 91, wherein at least 2 different Cas proteins are present in the ceDNA vector, and wherein one of the Cas protein is catalytically inactive (Cas-i), and wherein the guide RNA associated with the Cas-I targets the promoter of the target cell, and wherein the DNA coding for the Cas-I is under the control of an inducible promoter so that it can turn-off the expression of the target gene at a desired time.
97. A method for editing a single nucleotide base pair in a target gene of a cell, the method comprising contacting a cell with a CRISPR/Cas gene editing system, wherein one or more components of the CRISPR/Cas gene editing system are delivered to the cell by contacting the cell with a close-ended DNA (ceDNA) nucleic acid vector composition, wherein the ceDNA nucleic acid vector composition is a linear close-ended duplex DNA comprising flanking terminal repeat (TR) sequences and at least one gene editing nucleic acid sequence having a region complementary to at least one target gene or regulatory sequence for the target gene, and

wherein the Cas protein expressed from the vector is catalytically inactive and is fused to a base editing moiety,

wherein the method is performed under conditions and for a time sufficient to modulate expression of the target gene.

98. The method of any of paragraphs 79-97, wherein the terminal repeats are inverted TRs (ITRs).
99. The method of any of paragraphs 79-98, wherein at least one of the flanking terminal repeats is a modified terminal repeat.
100. The method of any of paragraphs 79-99, wherein the base editing moiety comprises a single-strand-specific cytidine deaminase, a uracil glycosylase inhibitor, or a tRNA adenosine deaminase.
101. The method of any of paragraphs 79-100, wherein the catalytically inactive Cas protein expressed from the vector is dCas9.
102. The method of any of paragraphs 79-101, wherein the ceDNA vector has the structure of any of paragraphs 1-77, wherein the cell contacted is a T cell, or CD34⁺.
103. The method of any of paragraphs 79-102, wherein the target gene encodes for a programmed death protein (PD1), cytotoxic T-lymphocyte-associated antigen 4 (CTLA4), or tumor necrosis factor-α (TNF-α).
104. The method of any of paragraphs 79-103, further comprising administering the cells produced by paragraph 102 to a subject in need thereof.
105. The method of paragraph 104, wherein the subject in need thereof has a genetic disease, viral infection, bacterial infection, cancer, or autoimmune disease.
106. A method of modulating expression of two or more target genes in a cell comprising: introducing into the cell:

(i) a composition comprising a vector that comprises: flanking terminal repeat (TR) sequences, and a nucleic acid sequence encoding at least two guide RNAs complementary to two or more target genes, wherein the vector is a linear close-ended duplex DNA,

(ii) a second composition comprising a vector that comprises: flanking terminal repeat (TR) sequences and a nucleic acid sequence encoding at least two catalytically inactive DNA endonucleases that each associate with a guide RNA and bind to the two or more target genes, wherein the vector is a linear close-ended duplex DNA, and

(iii) a third composition comprising a vector that comprises: flanking terminal repeat (TR) sequences, and a nucleic acid sequence encoding at least two transcriptional regulator proteins or domains, wherein the vector is a linear close-ended duplex DNA, and

wherein the at least two guide RNAs, the at least two catalytically inactive RNA-guided endonucleases and the at least two transcriptional regulator proteins or domains are expressed in the cell,

wherein two or more co-localization complexes form between a guide RNA, a catalytically inactive RNA-guided endonuclease, a transcriptional regulator protein or domain and a target gene, and

wherein the transcriptional regulator protein or domain regulates expression of the at least two target genes.

107. The method of paragraph 106, wherein the terminal repeats are inverted TRs (ITRs).
108. The method of paragraphs 106 or 107, wherein at least one of the flanking TR sequences is a modified TR.
109. A method for inserting a nucleic acid sequence into a genomic safe harbor gene, the method comprising: contacting a cell with (i) a gene editing system and (ii) a homology directed repair template having homology to a genomic safe harbor gene and comprising a nucleic acid sequence encoding a protein of interest,
wherein one or more components of the gene editing system are delivered to the cell by contacting the cell with a close-ended DNA (ceDNA) nucleic acid vector composition, wherein the ceDNA nucleic acid vector composition is a linear close-ended duplex DNA comprising flanking terminal repeat (TR) sequences and at least one gene editing nucleic acid sequence, and
wherein the method is performed under conditions and for a time sufficient to insert the nucleic acid sequence encoding the protein of interest into the genomic safe harbor gene.
110. The method of paragraph 109, wherein the terminal repeats are inverted TRs (ITRs).
111. The method of paragraphs 109 or 110, wherein at least one of the flanking TR sequences is a modified TR.
112. The method of any of paragraphs 109-111, wherein the genomic safe harbor gene comprises an active intron close to at least one coding sequence known to express proteins at a high expression level.
113. The method of any of paragraphs 109-112, wherein the ceDNA vector comprises a structure as in any one of paragraphs 1-77.
114. The method of any of paragraphs 109-113, wherein the genomic safe harbor gene comprises a site in or near the albumin gene.
115. The method of any of paragraphs 109-114, wherein the protein of interest is a receptor, a toxin, a hormone, an enzyme, or a cell surface protein.
116. The method of any of paragraphs 109-115, wherein, the protein of interest is a secreted protein.
117. The method of any of paragraphs 109-116, wherein the protein of interest comprises Factor VIII (FVIII) or Factor IX (FIX).
118. The method of any of paragraphs 109-117, wherein the method is performed in vivo for the treatment of hemophilia A, or hemophilia B.

### EXAMPLES

The following examples are provided by way of illustration not limitation. It will be appreciated by one of ordinary skill in the art that ceDNA vectors can be constructed from any of the wild-type or modified ITRs described herein, and that the following exemplary methods can be used to construct and assess the activity of such ceDNA vectors. While the methods are exemplified with certain ceDNA vectors, they are applicable to any ceDNA vector in keeping with the description.

### Example 1: Constructing ceDNA Vectors

Production of the ceDNA vectors using a polynucleotide construct template is described in Example 1 of PCT/US18/49996. For example, a polynucleotide construct template used for generating the ceDNA vectors of the present invention can be a ceDNA-plasmid, a ceDNA-Bacmid, and/or a ceDNA-baculovirus. Without being limited to theory, in a permissive host cell, in the presence of e.g., Rep, the polynucleotide construct template having two symmetric ITRs and an expression construct, where at least one of the ITRs is modified relative to a wild-type ITR sequence, replicates to produce ceDNA vectors. ceDNA vector production undergoes two steps: first, excision (“rescue”) of template from the template backbone (e.g. ceDNA-plasmid, ceDNA-bacmid, ceDNA-bacliovirus genome etc.) via Rep proteins, and second, Rep mediated replication of the excised ceDNA vector.

An exemplary method to produce ceDNA vectors is from a ceDNA-plasmid as described herein. Referring to FIGS. 1A and 1B, the polynucleotide construct template of each of the ceDNA-plasmids includes both a left modified ITR and a right modified ITR with the following between the ITR sequences: (i) an enhancer/promoter; (ii) a cloning site for a transgene; (iii) a posttranscriptional response element (e.g. the woodchuck hepatitis virus posttranscriptional regulatory element (WPRE)); and (iv) a poly-adenylation signal (e.g. from bovine growth hormone gene (BGHpA). Unique restriction endonuclease recognition sites (R1-R6) (shown in FIG. 1A and FIG. 1B) were also introduced between each component to facilitate the introduction of new genetic components into the specific sites in the construct. R3 (PmeI) GTTTAAAC (SEQ ID NO: 7) and R4 (Pad) TTAATTAA (SEQ ID NO: 542) enzyme sites are engineered into the cloning site to introduce an open reading frame of a transgene. These sequences were cloned into a pFastBac HT B plasmid obtained from ThermoFisher Scientific.

In brief, a series of ceDNA vectors for gene editing were obtained from ceDNA-plasmid constructs using the process shown in FIGS. 4A-4C. Table 8 shows exemplary constructs for generating gene editing ceDNA vectors for use herein, which can also comprise sequences, e.g., a replication protein site (RPS) (e.g. Rep binding site) on either end of a promoter operatively linked to the gene editing molecule. The numbers in Table 8 refer to SEQ ID NOs in this document, corresponding to the sequences of each component. The plasmids in Table 8 were constructed with the WPRE comprising SEQ ID NO: 8 followed by BGHpA comprising SEQ ID NO: 9 in the 3′ untranslated region between the transgene and the right side ITR.

TABLE 8
Exemplary constructs comprising an asymmetric ITR pair or a symmetric
mod-ITR pair for generation of exemplary gene editing ceDNA vectors.

| | | | 3′ modified ITR |
|---|---|---|---|
| | | | (symmetric |
| | | | relative to |
| Plasmid | 5′ modified ITR | Transgene | the 5′ ITR) |
| Constuct-1 | SEQ ID NO: 51 | Gene editing | SEQ ID NO: 2 |
| | | molecule | |
| Construct-2 | SEQ ID NO: 52 | Gene editing | SEQ ID NO: 1 |
| | | molecule | |
| Construct-3 | SEQ ID NO: 51 | Gene editing | SEQ ID NO: 2 |
| | | molecule | |
| Construct-4 | SEQ ID NO: 52 | Gene editing | SEQ ID NO: 1 |
| | | molecule | |
| Construct-5 | SEQ ID NO: 51 | Gene editing | SEQ ID NO: 2 |
| | | molecule | |
| Construct-6 | SEQ ID NO: 52 | Gene editing | SEQ ID NO: 1 |
| | | molecule | |
| Construct-7 | SEQ ID NO: 51 | Gene editing | SEQ ID NO: 2 |
| | | molecule | |
| Construct-8 | SEQ ID NO: 52 | Gene editing | SEQ ID NO: 1 |
| | | molecule | |
| Construct 11 | (SEQ ID NO: 63) | Gene editing | (SEQ ID NO: 1) |
| | | molecule | |
| Construct 12 | (SEQ ID NO: 51) | Gene editing | (SEQ ID NO: 64) |
| | | molecule | |
| Construct 13 | (SEQ ID NO: 63) | Gene editing | (SEQ ID NO: 1) |
| | | molecule | |
| Construct 14 | (SEQ ID NO: 51) | Gene editing | (SEQ ID NO: 64) |
| | | molecule | |
| Construct-15 | SEQ ID NO: 484 | Gene editing | SEQ ID NO: 469 |
| | (ITR-33 left) | molecule | (ITR-18, right) |
| Construct-16 | SEQ ID NO: 485 | Gene editing | SEQ ID NO: 95 |
| | (ITR-34 left) | molecule | (ITR-51, right) |
| Construct-17 | SEQ ID NO: 486 | Gene editing | SEQ ID NO: 470 |
| | (ITR-35 left) | molecule | (ITR-19, right) |
| Construct-18 | SEQ ID NO: 487 | Gene editing | SEQ ID NO: 471 |
| | (ITR-36 left) | molecule | (ITR-20, right) |
| Construct-19 | SEQ ID NO: 488 | Gene editing | SEQ ID NO: 472 |
| | (ITR-37 left) | molecule | (ITR-21, right) |
| Construct-20 | SEQ ID NO: 489 | Gene editing | SEQ ID NO: 473 |
| | (ITR-38 left) | molecule | (ITR-22 right) |
| Construct-21 | SEQ ID NO: 490 | Gene editing | SEQ ID NO: 474 |
| | (ITR-39 left) | molecule | (ITR-23, right) |
| Construct-22 | SEQ ID NO: 491 | Gene editing | SEQ ID NO: 475 |
| | (ITR-40 left) | molecule | (ITR-24, right) |
| Construct-23 | SEQ ID NO: 492 | Gene editing | SEQ ID NO: 476 |
| | (ITR-41 left) | molecule | (ITR-25 right) |
| Construct-24 | SEQ ID NO: 493 | Gene editing | SEQ ID NO: 477 |
| | (ITR-42 left) | molecule | (ITR-26 right) |
| Construct-25 | SEQ ID NO: 494 | Gene editing | SEQ ID NO: 478 |
| | (ITR-43 left) | molecule | (ITR-27 right) |
| Construct-26 | SEQ ID NO: 495 | Gene editing | SEQ ID NO: 479 |
| | (ITR-44 left) | molecule | (ITR-28 right) |
| Construct-27 | SEQ ID NO: 496 | Gene editing | SEQ ID NO: 480 |
| | (ITR-45 left) | molecule | (ITR-29, right) |
| Construct-28 | SEQ ID NO: 497 | Gene editing | SEQ ID NO: 481 |
| | (ITR-46 left) | molecule | (ITR-30, right) |
| Construct-29 | SEQ ID NO: 498 | Gene editing | SEQ ID NO: 482 |
| | (ITR-47, left) | molecule | (ITR-31, right) |
| Construct-30 | SEQ ID NO: 499 | Gene editing | SEQ ID NO: 483 |
| | (ITR-48, left) | molecule | (ITR-32 right) |
| Construct-31 | SEQ ID NO: 51 | Gene editing | SEQ ID NO: 1 |
| | (WT-ITR) | molecule | (WT-ITR) |

In some embodiments, a construct to make a gene editing ceDNA vectors comprises a promoter which is a regulatory switch as described herein, e.g., an inducible promoter.

Production of ceDNA-Bacmids:

With reference to FIG. 4A, DH10Bac competent cells (MAX EFFICIENCY® DH10Bac™ Competent Cells, Thermo Fisher) were transformed with either test or control plasmids following a protocol according to the manufacturer's instructions. Recombination between the plasmid and a baculovirus shuttle vector in the DH10Bac cells were induced to generate recombinant ceDNA-bacmids. The recombinant bacmids were selected by screening a positive selection based on blue-white screening in E. coli (Φ80dlacZΔM15 marker provides a-complementation of the β-galactosidase gene from the bacmid vector) on a bacterial agar plate containing X-gal and IPTG with antibiotics to select for transformants and maintenance of the bacmid and transposase plasmids. White colonies caused by transposition that disrupts the β-galactoside indicator gene were picked and cultured in 10 ml of media.

The recombinant ceDNA-bacmids were isolated from the E. coli and transfected into Sf9 or Sf21 insect cells using FugeneHD to produce infectious baculovirus. The adherent Sf9 or Sf21 insect cells were cultured in 50 ml of media in T25 flasks at 25° C. Four days later, culture medium (containing the P0 virus) was removed from the cells, filtered through a 0.45 μm filter, separating the infectious baculovirus particles from cells or cell debris.

Optionally, the first generation of the baculovirus (P0) was amplified by infecting naïve Sf9 or Sf21 insect cells in 50 to 500 ml of media. Cells were maintained in suspension cultures in an orbital shaker incubator at 130 rpm at 25° C., monitoring cell diameter and viability, until cells reach a diameter of 18-19 nm (from a naïve diameter of 14-15 nm), and a density of ˜4.0E+6 cells/mL. Between 3 and 8 days post-infection, the P1 baculovirus particles in the medium were collected following centrifugation to remove cells and debris then filtration through a 0.45 μm filter.

The ceDNA-baculovirus comprising the test constructs were collected and the infectious activity, or titer, of the baculovirus was determined. Specifically, four×20 ml Sf9 cell cultures at 2.5E+6 cells/ml were treated with P1 baculovirus at the following dilutions: 1/1000, 1/10,000, 1/50,000, 1/100,000, and incubated at 25-27° C. Infectivity was determined by the rate of cell diameter increase and cell cycle arrest, and change in cell viability every day for 4 to 5 days.

With reference to FIG. 4A, a “Rep-plasmid” according to, e.g., FIG. 7A was produced in a pFASTBAC™-Dual expression vector (ThermoFisher) comprising both the Rep78 (SEQ ID NO: 13) or Rep68 (SEQ ID NO: 12) and Rep52 (SEQ ID NO: 14) or Rep40 (SEQ ID NO: 11).

The Rep-plasmid was transformed into the DH10Bac competent cells (MAX EFFICIENCY® DH10Bac™ Competent Cells (Thermo Fisher) following a protocol provided by the manufacturer. Recombination between the Rep-plasmid and a baculovirus shuttle vector in the DH10Bac cells were induced to generate recombinant bacmids (“Rep-bacmids”). The recombinant bacmids were selected by a positive selection that included-blue-white screening in E. coli (Φ80dlacZΔM15 marker provides a-complementation of the β-galactosidase gene from the bacmid vector) on a bacterial agar plate containing X-gal and IPTG. Isolated white colonies were picked and inoculated in 10 ml of selection media (kanamycin, gentamicin, tetracycline in LB broth). The recombinant bacmids (Rep-bacmids) were isolated from the E. coli and the Rep-bacmids were transfected into Sf9 or Sf21 insect cells to produce infectious baculovirus.

The Sf9 or Sf21 insect cells were cultured in 50 ml of media for 4 days, and infectious recombinant baculovirus (“Rep-baculovirus”) were isolated from the culture. Optionally, the first generation Rep-baculovirus (P0) were amplified by infecting naïve Sf9 or Sf21 insect cells and cultured in 50 to 500 ml of media. Between 3 and 8 days post-infection, the P1 baculovirus particles in the medium were collected either by separating cells by centrifugation or filtration or another fractionation process. The Rep-baculovirus were collected and the infectious activity of the baculovirus was determined. Specifically, four x 20 mL Sf9 cell cultures at 2.5×10⁶ cells/mL were treated with P1 baculovirus at the following dilutions, 1/1000, 1/10,000, 1/50,000, 1/100,000, and incubated. Infectivity was determined by the rate of cell diameter increase and cell cycle arrest, and change in cell viability every day for 4 to 5 days.

ceDNA Vector Generation and Characterization

With reference to FIG. 4B, Sf9 insect cell culture media containing either (1) a sample-containing a ceDNA-bacmid or a ceDNA-baculovirus, and (2) Rep-baculovirus described above were then added to a fresh culture of Sf9 cells (2.5E+6 cells/ml, 20 ml) at a ratio of 1:1000 and 1:10,000, respectively. The cells were then cultured at 130 rpm at 25° C. 4-5 days after the coinfection, cell diameter and viability are detected. When cell diameters reached 18-20 nm with a viability of ˜70-80%, the cell cultures were centrifuged, the medium was removed, and the cell pellets were collected. The cell pellets are first resuspended in an adequate volume of aqueous medium, either water or buffer. The ceDNA vector was isolated and purified from the cells using Qiagen MIDI PLUS™ purification protocol (Qiagen, 0.2 mg of cell pellet mass processed per column).

Yields of ceDNA vectors produced and purified from the Sf9 insect cells were initially determined based on UV absorbance at 260 nm.

ceDNA vectors can be assessed by identified by agarose gel electrophoresis under native or denaturing conditions as illustrated in FIG. 4D, where (a) the presence of characteristic bands migrating at twice the size on denaturing gels versus native gels after restriction endonuclease cleavage and gel electrophoretic analysis and (b) the presence of monomer and dimer (2×) bands on denaturing gels for uncleaved material is characteristic of the presence of ceDNA vector.

Structures of the isolated ceDNA vectors were further analyzed by digesting the DNA obtained from co-infected Sf9 cells (as described herein) with restriction endonucleases selected for a) the presence of only a single cut site within the ceDNA vectors, and b) resulting fragments that were large enough to be seen clearly when fractionated on a 0.8% denaturing agarose gel (>800 bp). As illustrated in FIGS. 4D and 4E, linear DNA vectors with a non-continuous structure and ceDNA vector with the linear and continuous structure can be distinguished by sizes of their reaction products—for example, a DNA vector with a non-continuous structure is expected to produce 1 kb and 2 kb fragments, while a non-encapsidated vector with the continuous structure is expected to produce 2 kb and 4 kb fragments.

Therefore, to demonstrate in a qualitative fashion that isolated ceDNA vectors are covalently closed-ended as is required by definition, the samples were digested with a restriction endonuclease identified in the context of the specific DNA vector sequence as having a single restriction site, preferably resulting in two cleavage products of unequal size (e.g., 1000 bp and 2000 bp). Following digestion and electrophoresis on a denaturing gel (which separates the two complementary DNA strands), a linear, non-covalently closed DNA will resolve at sizes 1000 bp and 2000 bp, while a covalently closed DNA (i.e., a ceDNA vector) will resolve at 2x sizes (2000 bp and 4000 bp), as the two DNA strands are linked and are now unfolded and twice the length (though single stranded). Furthermore, digestion of monomeric, dimeric, and n-meric forms of the DNA vectors will all resolve as the same size fragments due to the end-to-end linking of the multimeric DNA vectors (see FIG. 4D).

As used herein, the phrase “assay for the Identification of DNA vectors by agarose gel electrophoresis under native gel and denaturing conditions” refers to an assay to assess the close-endedness of the ceDNA by performing restriction endonuclease digestion followed by electrophoretic assessment of the digest products. One such exemplary assay follows, though one of ordinary skill in the art will appreciate that many art-known variations on this example are possible. The restriction endonuclease is selected to be a single cut enzyme for the ceDNA vector of interest that will generate products of approximately ⅓× and ⅔× of the DNA vector length. This resolves the bands on both native and denaturing gels. Before denaturation, it is important to remove the buffer from the sample. The Qiagen PCR clean-up kit or desalting “spin columns,” e.g. GE HEALTHCARE ILUSTRA™ MICROSPIN™ G-25 columns are some art-known options for the endonuclease digestion. The assay includes for example, i) digest DNA with appropriate restriction endonuclease(s), 2) apply to e.g., a Qiagen PCR clean-up kit, elute with distilled water, iii) adding 10× denaturing solution (10×=0.5 M NaOH, 10 mM EDTA), add 10× dye, not buffered, and analyzing, together with DNA ladders prepared by adding 10× denaturing solution to 4×, on a 0.8-1.0% gel previously incubated with 1 mM EDTA and 200 mM NaOH to ensure that the NaOH concentration is uniform in the gel and gel box, and running the gel in the presence of 1× denaturing solution (50 mM NaOH, 1 mM EDTA). One of ordinary skill in the art will appreciate what voltage to use to run the electrophoresis based on size and desired timing of results. After electrophoresis, the gels are drained and neutralized in 1×TBE or TAE and transferred to distilled water or 1×TBE/TAE with 1×SYBR Gold. Bands can then be visualized with e.g. Thermo Fisher, SYBR® Gold Nucleic Acid Gel Stain (10,000× Concentrate in DMSO) and epifluorescent light (blue) or UV (312 nm).

The purity of the generated ceDNA vector can be assessed using any art-known method. As one exemplary and nonlimiting method, contribution of ceDNA-plasmid to the overall UV absorbance of a sample can be estimated by comparing the fluorescent intensity of ceDNA vector to a standard. For example, if based on UV absorbance 4 μg of ceDNA vector was loaded on the gel, and the ceDNA vector fluorescent intensity is equivalent to a 2 kb band which is known to be 1 μg, then there is 1 μg of ceDNA vector, and the ceDNA vector is 25% of the total UV absorbing material. Band intensity on the gel is then plotted against the calculated input that band represents—for example, if the total ceDNA vector is 8 kb, and the excised comparative band is 2 kb, then the band intensity would be plotted as 25% of the total input, which in this case would be 0.25 μg for 1.0 μg input. Using the ceDNA vector plasmid titration to plot a standard curve, a regression line equation is then used to calculate the quantity of the ceDNA vector band, which can then be used to determine the percent of total input represented by the ceDNA vector, or percent purity.

### Example 2: ceDNA Vectors Express Luciferase Transgene In Vitro

Constructs were generated by introducing an open reading frame encoding the Luciferase reporter gene into the cloning site of ceDNA-plasmid constructs: construct-15-30, (see above in Table 8) including the Luciferase coding sequence. HEK293 cells were cultured and transfected with 100 ng, 200 ng, or 400 ng of plasmid constructs 1-31, using FUGENE® (Promega Corp.) as a transfection agent. Expression of Luciferase from each of the plasmids was determined based on Luciferase activity in each cell culture, confirming that the Luciferase activity resulted from gene expression from the plasmids.

### Example 3: In Vivo Protein Expression of Luciferase Transgene from ceDNA Vectors

In vivo protein expression of a transgene from ceDNA vectors produced from the constructs can be assessed in mice. For example, the ceDNA vectors obtained from ceDNA-plasmid constructs 1-31 (as described in Table 8) were tested and demonstrated sustained and durable luciferase transgene expression in a mouse model following hydrodynamic injection of the ceDNA construct without a liposome, redose (at day 28) and durability (up to Day 42) of exogenous firefly luciferase ceDNA. In different experiments, the luciferase expression of selected ceDNA vectors is assessed in vivo, where the ceDNA vectors comprise the luciferase transgene and a 5′ ITR and a 3′ITR are selected from any ITR pair listed in any of Table 2, Table 4A, Table 4B or Table 5, or any of the modified ITR pairs shown in FIGS. 7A-7B. The following exemplary methods have been used to assess in vivo protein expression from ceDNA vectors.

In Vivo Luciferase Expression:

5-7 week male CD-1 IGS mice (Charles River Laboratories) are administered 0.35 mg/kg of ceDNA vector expressing luciferase in 1.2 mL volume via i.v. hydrodynamic administration to the tail vein on Day 0. Luciferase expression is assessed by IVIS imaging on Day 3, 4, 7, 14, 21, 28, 31, 35, and 42. Briefly, mice are injected intraperitoneally with 150 mg/kg of luciferin substrate and then whole body luminescence was assessed via IVIS® imaging.

IVIS imaging is performed on Day 3, Day 4, Day 7, Day 14, Day 21, Day 28, Day 31, Day 35, and Day 42, and collected organs are imaged ex vivo following sacrifice on Day 42.

During the course of the study, animals are weighed and monitored daily for general health and well-being. At sacrifice, blood is collected from each animal by terminal cardiac stick, and split into two portions and processed to 1) plasma and 2) serum, with plasma snap-frozen and serum used for liver enzyme panel and subsequently snap frozen. Additionally, livers, spleens, kidneys, and inguinal lymph nodes (LNs) are collected and imaged ex vivo by IVIS.

Luciferase expression is assessed in livers by MAXDISCOVERY® Luciferase ELISA assay (BIOO Scientific/PerkinElmer), qPCR for Luciferase of liver samples, histopathology of liver samples and/or a serum liver enzyme panel (VetScanVS2; Abaxis Preventative Care Profile Plus).

### Example 4: Modified ITR Screening

A. Modified ITR Screening for ceDNA Vectors Comprising Asymmetric and Symmetric ITR Pairs.

The analysis of the relationship of mod-ITR structure to ceDNA formation can be performed as described in PCT application PCT/US18/49996 which is incorporated herein in its entirety by reference. A series of mod-ITRs as shown in FIGS. 7A-7B and Table 4A and 4B herein were constructed to query the impact of specific structural changes on ceDNA formation and ability to express the ceDNA-encoded transgene. Mutant construction, assay of ceDNA formation, and assessment of ceDNA transgene expression in human cell culture are described in further detail below. As expected, the three negative controls (media only, mock transfection lacking donor DNA, and sample that was processed in the absence of Rep-containing baculovirus cells) showed no significant luciferase expression. Robust luciferase expression was observed in each of the mutant samples, indicating that for each sample the ceDNA-encoded transgene was successfully transfected and expressed irrespective of the mutation. Thus, the mutant samples appeared to correctly form ceDNA comprising asymmetrical mod-ITR pair. Mod-ITR may be used in the compositions and methods of the invention and can be screened for activity using the following exemplary methods.

ceDNA vectors with symmetric ITR pairs were generated and constructed as described in Example 1 above and described in FIG. 4B. Analysis of the relationship of symmetric mod-ITR and symmetric WT-ITRs was assessed according to the methods as described in PCT/US18/49996 which is incorporated herein in its entirety by reference. Mutations to the ITR sequence were created symmetrically on both the right and left ITR regions. The library contained 16 right-sided double mutants (e.g., symmetrical mod-ITR pairs), as disclosed in Table 5.

### Example 5: Generation of a Gene Editing ceDNA Vector

For illustrative purposes, an exemplary gene editing ceDNA vector is described with respect to generating a ceDNA vector for editing the Factor VIII, and is described below. However, while Factor VIII is exemplified in this Example to illustrate methods to generate a gene editing ceDNA vector useful in the methods and constructs as described herein, one of ordinary skill in the art is aware that one can, as stated above use, use any gene where gene editing is desired. Exemplary genes for editing are described herein, for example, in the sections entitled “Exemplary diseases to be treated with a gene editing ceDNA” and “additional diseases for gene editing”.

Generation of a Factor VIII gene editing ceDNA: an open reading frame including a transgene of interest (e.g., as one nonlimiting example, Factor VIII) is inserted into the ceDNA vector, flanked by large (up to 2 Kb each) homology arms of the genomic DNA sequence adjacent to the open reading frame to facilitate HDR within the endogenous transgene locus for patients having a disease or disorder associated with a defective native copy of the transgene (in the case of Factor VIII, patients afflicted with Hemophilia A). A site-specific nuclease open reading frame is optionally included in the vector, along with any needed adjunct components such as an sgRNA, with the nuclease specific for a site at or near the native transgene locus (e.g., the Factor VIII locus) and effective to increase recombination. the ceDNA vector may also be engineered such that the nuclease is further specific for sites on the ceDNA vector itself that disable the expression of nuclease from the ceDNA vector. Such further specificity is provided by further gRNAs expressed by the ceDNA vector. The ceDNA may be delivered in, e.g., lipid nanoparticles (LNPs) as described herein.

A ceDNA-transgene construct can be further engineered to include a nuclease (e.g., Cas9, TALEs, MegaTales, or ZFNs) and, if necessary the guide RNA that provides the DNA specificity to the gene editing process. Therefore, this ‘all-in-one’ ceDNA construct has the following elements in addition to the core ceDNA backbone elements: a transgene coding sequence (e.g., a transgene encoding Factor VIII); two genomic homology regions (e.g., HRs specific for the endogenous Factor VIII locus); a nuclease coding region and a promoter for driving expression of the nuclease; and, in the case where a CRISPR system is being utilized, a guide RNA (e.g., in the case of Cas9). One can engineer the ceDNA vector such that it has the sgRNA and Cas9 expression cassettes in cis with the transgene and the sgRNA and Cas9 are outside of the homology arms and therefore are not integrated into the cellular genome. After the gene editing event, the linear ceDNA after HDR will have exposed DNA ends and therefore will be degraded, thus reducing the expression from this construct.

An exemplary ceDNA vector having a Factor VIII construct can be further modified to have a DNA sequence engineered into the nuclease sequence (or its promoter) that will induce its own inactivation. For example, when Cas9 protein is produced, it will not only induce gene editing (i.e., the desired effect), but it will also bind to and induce a double strand DNA break within the ceDNA thus ensuring the downregulation/elimination of Cas9 (to reduce the chance of off-target DNA breaks induced by persistent Cas9).

A gene editing ceDNA vector encoding Factor VIII can be generated with genomic homology arms to the albumin locus or other genomic loci (near a strong promoter to drive expression of the inserted Factor VIII). The various experiments recited in Example 5 are repeated in this framework.

FIG. 10A shows a test-vector expression unit in accordance with the present disclosure, flanked by 5′ and 3′ homology arms that is incorporated into the ceDNA design. In this embodiment, a ceDNA is designed with a Factor IX (FIX) open reading frame flanked with 5′ and 3′ homology arms that hybridize to the Albumin genomic locus and therefore drive expression of the FIX under the endogenous Albumin promoter. Controls are an expression unit only the 5′ homology arm; and one containing only the 3′ homology arm (FIGS. 10B and 10C respectively). An expression unit a reporter gene, e.g., GFP, including a promoter, WPRE element, pA, can be used to experimentally confirm expression (FIG. 10D).

A ceDNA vector comprising a nuclease expressing unit can be delivered in trans, such Cas9 mRNA, zinc-finger nucleases (ZFN), transcription activator-like effector nucleases (TALEN), mutated “nickase” endonuclease, class II CRISPR/Cas system (CPF1) (FIG. 10E). LNPs as decribed herein can be used as a delivery option. Transport of the nuclease expressing unit to the nuclei can be increased or improved by using a nuclear localization signal (NLS) fused into the 5′ or 3′ enzyme peptide sequence (e.g., the nuclease expressing unit, such as Cas9, ZFN, TALEN etc). Depending on the nuclease expressed by the ceDNA, to induce double-stranded break (DSB) at the desired site, one or more single guided RNA can also be delivered in trans. For example, either as an sgRNA expressing vector or chemically synthesized synthetic sgRNA. (sg=single guide-RNA target sequence) (FIG. 10F). The sgRNA vector can be a ceDNA vector or other expression vector. Single-guide RNA sequences can be selected and validated using freely available software/algorithm. 4 potential candidate sequences are selected and validated. (Public resources, such as at tools.genome-engineering.org can be used to select suitable single guide-RNA sequences.)

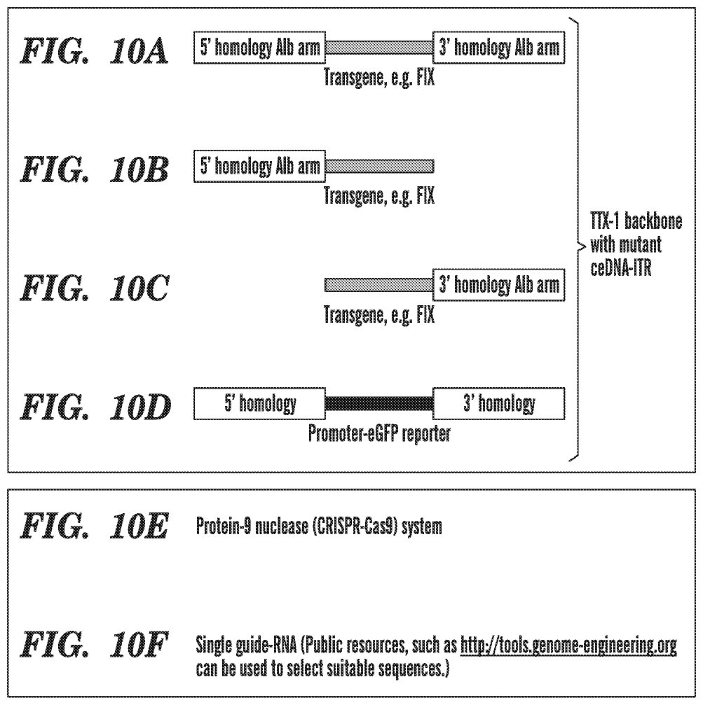

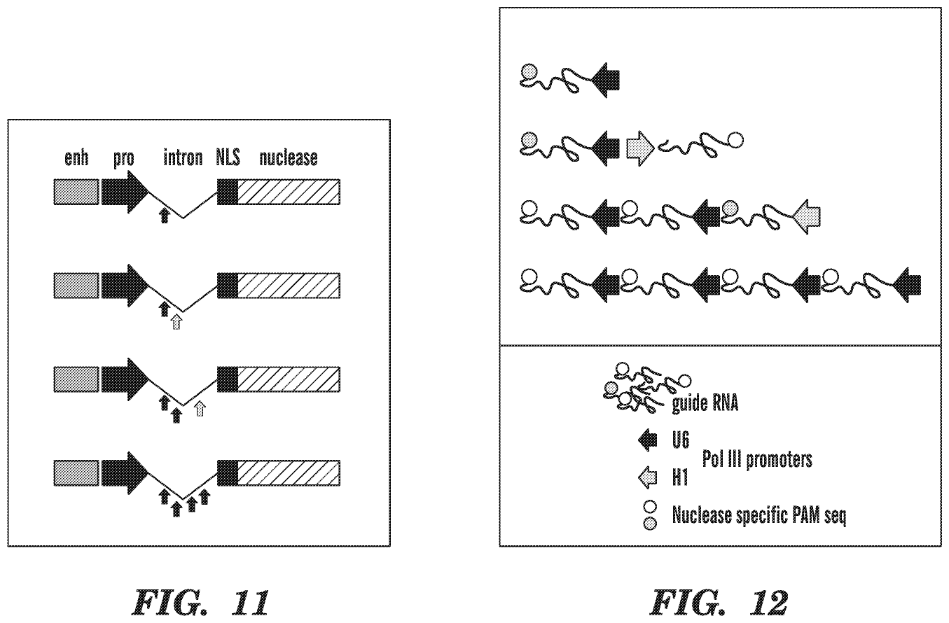

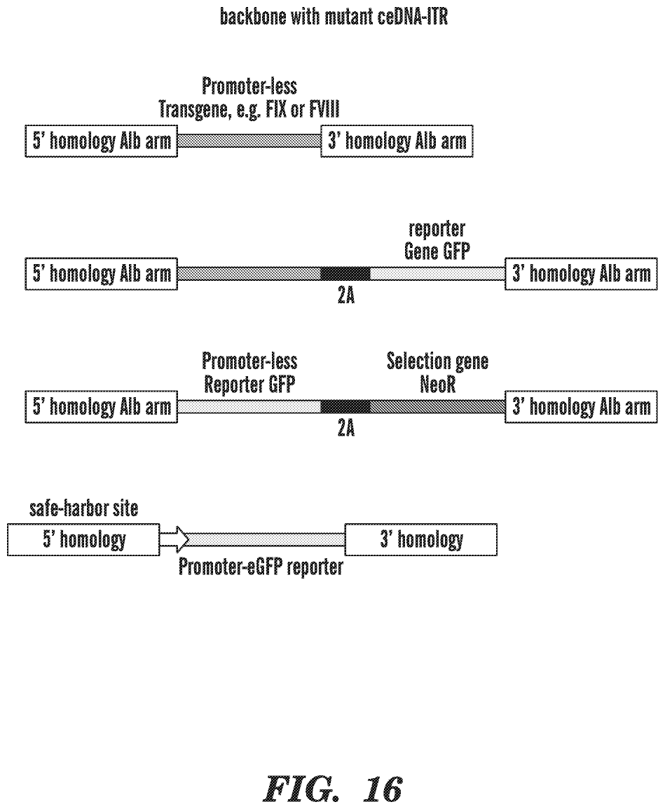

Exemplary 5′ and 3′ homology arms: a 5′ and/or 3′ homology arm can be about 350 bp long, for use in ceDNA constructs as depicted in FIGS. 8, 9 and 10A-10F. For example, the 5′ homology arm can range between 50 to 2000 bp. Similarity, a 3′ homology arm can be about 2000 bp long, and can be in the range of between 50 to 2000 bp. One of ordinary skill in the art can modify the length of 5′ and/or 3′ homology arm and/or recombination frequency as described in Zhang, Jian-Ping, et al. “Efficient precise knockin with a double cut HDR donor after CRISPR/Cas9-mediated double-stranded DNA cleavage,” Genome biology 18.1 (2017): 35. and Wang, Yuanming, et al. “Systematic evaluation of CRISPR-Cas systems reveals design principles for genome editing in human cells.” Genome biology 19.1 (2018): 62. As shown herein in FIG. 16, FIX or FVIII can be substituted with any promoter-less open-reading frame (ORF). Additional elements, including but not limited to, WPRE and polyadenylation signal, such as BGHpA can be added to the gene editing ceDNA construct. For example, expression of the gene to be inserted (e.g., FIX or FVII as exemplary genes) is driven by the endogenous and very strong Albumin promoter. A transcription enhancing element, such as WPRE is added 3′ of the ORF. A polyadenylation signal (e.g., BGH-pA) can also be added. As disclosed herein, the capacity of the ceDNA constructs is large therefore allowing the length of the DNA fragment between the ITRs to be above 15 kb. Accordingly, ceDNA vectors systems with large ORFs are encompassed for use. Also, other expression units with a strong promoter unit can be used. ceDNA vectors with homology arms that target other safe harbor locus can be used, e.g., have homology arms that instead of targeting the albumin locus, target other safe harbor locuses, such as, but not limited to the CCR5 or AAV-safe-harbor-S1 (AAVS1) locus. This allows one to insert the gene editing molecule or target gene into an intron site without any effects on the target cell or tissue. As shown in FIG. 11, expression constructs can be made for titration of self-inactivating features of the nuclease activity by introducing sgRNA sequences in the intron of the synthetic promoter unit, e.g., the CAG promoter described in the ceDNA vector. The degree of inactivation is regulated by the number of sgRNA seq or combination and/or mutated (de-optimized) sgRNA target seq. (Zhang et al, NatPro, 2013 Regulation of Cas9 activity by using de-optimized sgRNA recognition target sequence.) In FIG. 11, sgRNAs are alone or in multiples (e.g., four), and in some embodiments, can consist of one or multiple unique target sequences, represented by different black or white.

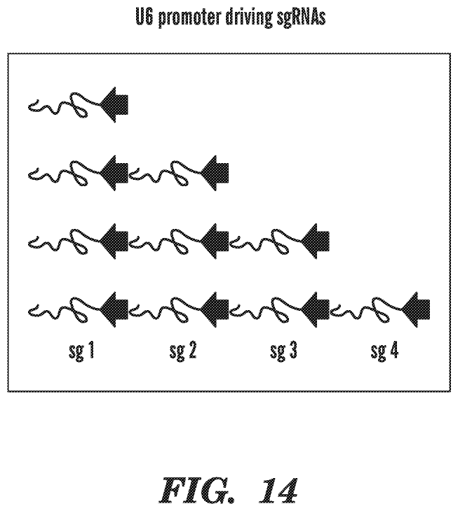

FIG. 12 shows an example where the ceDNA vector can comprise various Pol III promoter unit arrangements to drive the expression of one or more sgRNAs. In this example, more than one promoter of choice placed between the ITRs. The transcription direction can be in forward or reverse orientation. The sgRNAs can be combined and/or duplicated. FIG. 14 shows another example where a ceDNA can express multiple sgRNAs (sg1, sg2, sg3, or sg4), such as utilizing the U6 promoter.

Accordingly, ceDNA vectors for gene editing for use herein can comprise any one or more of these modifications.

### Example 6: All-In-One Gene Editing ceDNA Vector with Master ORF

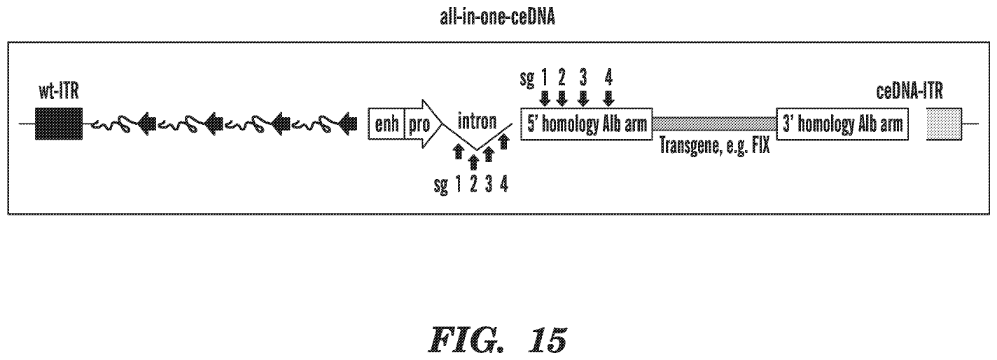

A gene editing ceDNA vector can be made containing the features as shown in FIG. 15. An included feature not labeled is a nuclease expression unit (including hashed nuclease element) and an intron downstream of the promoter having the illustrated sgRNA targeting sequence. The features include an ceDNA specific ITR; Pol III promoter driven (U6 or H1) sgRNA expressing unit with optional orientation in regard the transcription direction; Synthetic promoter driven nuclease (e.g., Cas9, double mutant Nickase, Talen, or other mutants) expression unit that may contain sgRNA targeting sequences with or w/o de-optimization (in experiments, located other than as indicated); A transgene (e.g., FIX) potentially fused to a selection marker (e.g., NeoR) through a viral 2A peptide cleavage site (2A) flanked by 0.05 to 6 kb stretching homology arms. (On 2A systems: Chan et al, Comparison of IRES and F2A-Based Locus-Specific Multicistronic Expression in Stable Mouse LinesHSV-TK suicide, PLOS 2011 HSV-TK suicide gene system; Fesnak et al, Engineered T Cells: The Promise and Challenges of Cancer Immunotherapy, Nat Rev Can 2016.)

If suitable, a negative selection marker (e.g., HSV TK) and expressing unit that allows one to control and selected for successful correct site usage, positioned outside of the homology arms is envisioned. Other Regulatory elements or Regulatory switches as disclosed herein are also encompassed in place of, or supplemental to the negative selection marker gene.

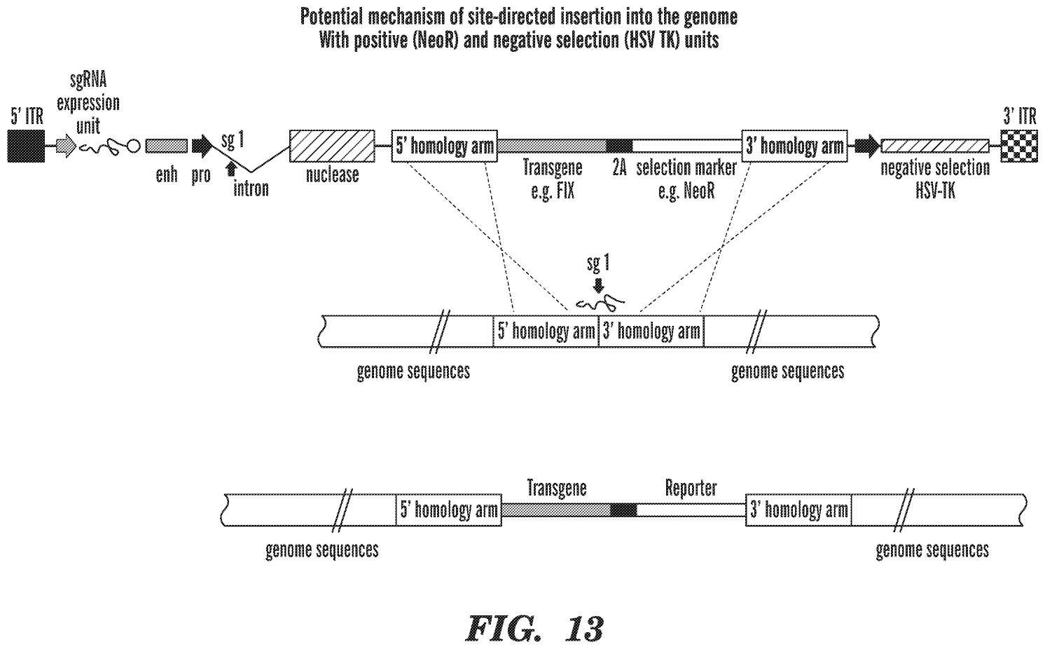

An exemplary ceDNA vector comprising homology arms for insertion of the HDR element is shown in FIG. 13. In such a ceDNA vector, if there is random integration, the entire vector with negative selectable marker is integrated into the genome. Such mis-transfected cells can be killed with appropriate drugs, such as GVC for the HSV TK negative selectable marker. Alternatively, the negative selectable marked can be replaced with a regulatory switch as described herein, e.g., a kill switch gene or any gene disclosed in Table 11 of PCT/US18/49996, which is incorporated herein in its entirety by reference.

Another exemplary ceDNA vector is shown in FIG. 9 that is similar to that of this Example, but replaces the negative selection marker with a sgRNA target seq for “double mutant nickase” (indicated by solid downward arrow point). The introduction of single stranded DNA cut (nicking) can help to release torsion downstream of the 3′ homology arm close to the mutant ITR and increase annealing and therefore increase HDR frequency. In such a ceDNA vector, the negative marker is used with the sgRNA target sequence for “double mutant nickase.”

The ceDNA vectors discussed in this Example are for illustrative purposes only, and can be modified to by an ordinary skilled artisan to insert different target genes, e.g., instead of FIX being used, Factor XIII is used, and where Factor XIII is used, FIX is used. Similarly, one of ordinary skill in the art is aware that one can use any target gene where gene editing is desired.

### Example 7: Generation of a Gene Editing ceDNA Vector for Treatment of Disease

For illustrative purposes, Example 7 describes generating exemplary gene editing ceDNA vectors for treating different diseases. However, while genes for cystic fibrosis, liver disorders, systemic disorders, CNS disorders and muscle disorders are exemplified in this Example to illustrate methods to generate a gene editing ceDNA vector useful in the methods and constructs as described herein, one of ordinary skill in the art is aware that one can, as stated above use, modify the target gene to treat any disease where gene editing is desired. Exemplary diseases or genetic disorders where gene editing is a desired strategy to treat a disease with a ceDNA editing vector as described herein is discussed in the sections entitled “Exemplary diseases to be treated with a gene editing ceDNA” and “additional diseases for gene editing”.

In one example, a ceDNA vector can be generated that comprises a sgRNA with multiple nuclease cleavage sites, such as 2-4, are put into one or both of an upstream intron for the nuclease and the 5′ homology arm. These can have specificity driven by distinct or shared sgRNAs. An exemplary “All-In-One” ceDNA vector having all of these features is shown in FIG. 15.

An exemplary transgene replacing or providing ceDNA vector can be configured to induce gene editing with distinct transgenes for other genetic disorders, including liver disorders (e.g., OTC, GSD 1 a, Crigler-Najar, PKU, and the like) or systemic disorders (e.g., MPSII, MLD, MPSIIIA, Gaucher, Fabry, Pompe, and the like).

An example of a gene editing ceDNA vector for treating a genetic disorder or disease can be similar to that discussed in Examples 6, in that the ceDNA vector can be modified to induce gene editing in the lung, for example in Cystic Fibrosis (CF). Such a ceDNA vector is created to encode CFTR, the gene that is mutated in CF. CFTR is a large gene that cannot be comprised within AAV. Therefore, a ceDNA vector provides a unique solution and can, in some embodiments, be administered intravenously and/or as a nebulized formulation to a subject to induce gene editing of lung epithelia. As above, a ceDNA gene editing vector is configured such that CFTR is inserted into the endogenous CFTR locus. In such an example, the ceDNA vector can also comprise the nuclease and guide RNA as well as, utilizing large homology arms to increase the efficiency and fidelity of gene editing.

An example of a gene editing ceDNA vector for gene editing of CNS disorders is similar to that discussed in Example 6, where the ceDNA is modified to induce gene editing in the CNS, for disorders including neurodegenerative disorders (e.g., familial forms of Alzheimer's, Parkinson's, Huntington's), lysosomal storage disorders (e.g., MPSII, MLD, MPSIIIA, Canavan, Batten, and the like) or neurodevelopmental disorders (e.g., SMA, Rett syndrome, and the like)

An example of a gene editing ceDNA vector for treating a genetic disorder or disease of the muscles can be similar to that discussed in Examples 6, in that the ceDNA vector can be modified to induce gene editing in the muscle, for disorders including but not limited to Duchenne muscular dystrophy, fascioscapulohumeral dystrophy, and the like.

A gene editing ceDNA (i.e., a transgene replacing or providing ceDNA vector) discussed and exemplified in Examples 6-7 can be delivered to target cells in an animal model for the defective transgene to assess the efficacy of the gene editing and also to provide cells that produce more effective gene product.

### Example 8: ceDNA is Suitable for Use in Gene Editing where a Meganuclease Performs a Targeted Double Strand Break (DSB)

A gene editing ceDNA vector can comprise a template nucleotide sequence as a correcting DNA strand to be inserted after a double-strand break provided by a meganuclease. For illustrative purposes, an exemplary gene editing ceDNA vector is described with respect to generating a ceDNA vector for editing and correcting the Apo A-I gene, and is described below. However, while correction of Apo A-I gene is exemplified in this Example to illustrate methods to generate a gene editing ceDNA vector useful in the methods and constructs as described herein, one of ordinary skill in the art is aware that one can, as stated above use the ceDNA vectors to correct the sequence of any other gene where gene editing is desired. Exemplary genes for editing are described herein, for example, in the sections entitled “Exemplary diseases to be treated with a gene editing ceDNA” and “additional diseases for gene editing”.

Meganuclease-Induced Correction of a Mutated Human ApoAI Gene In Vivo

Use of double stranded break (DSB) induced gene conversion in mammal in vivo by direct injection of a mixture of meganuclease expression cassette and ceDNA in the blood stream is performed. A system is provided based on the repair of a human Apo A-I transgene in mice in vivo. The apolipoprotein A-I (APO A-I) is the main protein constituent of high density lipoprotein (HDL) and plays an important role in HDL metabolism. High density lipoproteins have a major cardio-protective role as the principal mediator of the reverse cholesterol transport. The Apo A-I gene is expressed in the liver and the protein is secreted in the blood. Moreover, Apo A-I deficiency in human leads to premature coronary heart disease. All together, these criteria make Apo A-I gene a good candidate for the study of meganuclease-induced gene correction including ceDNA.

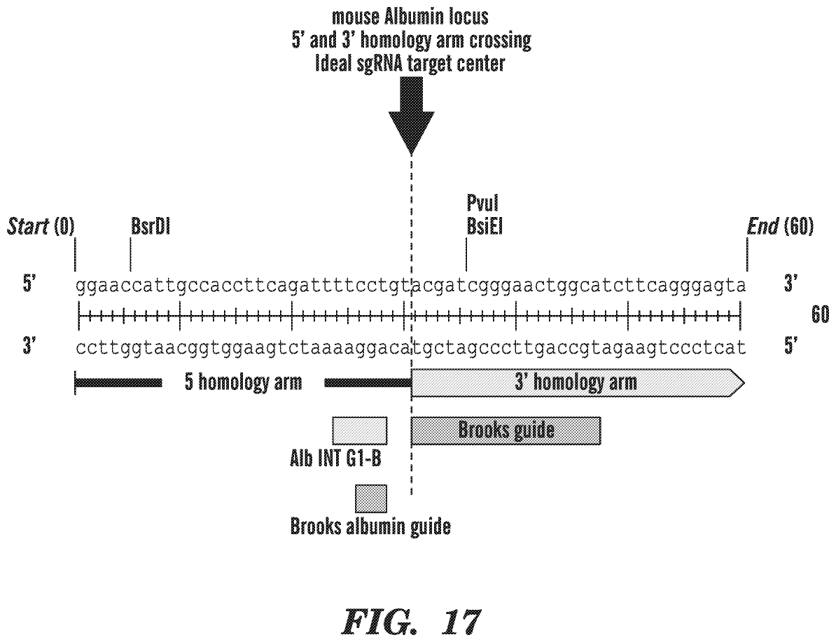

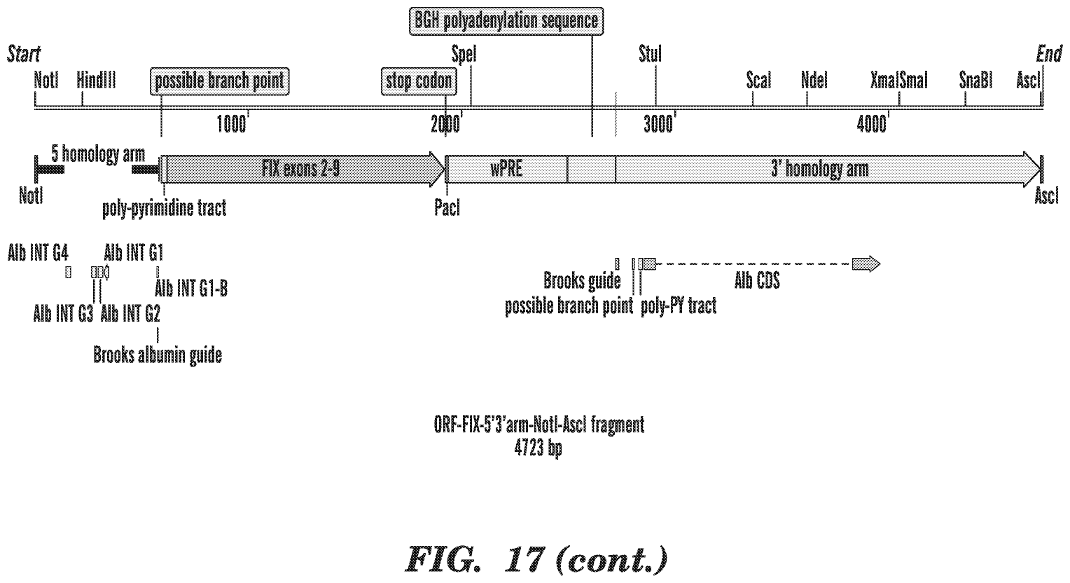

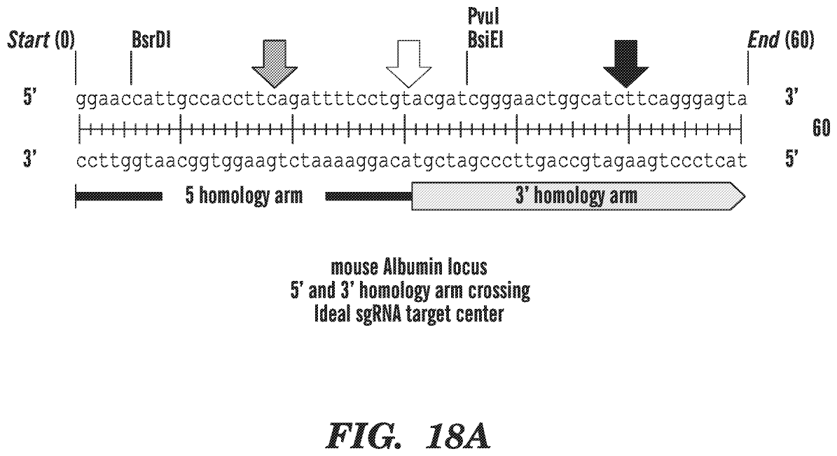

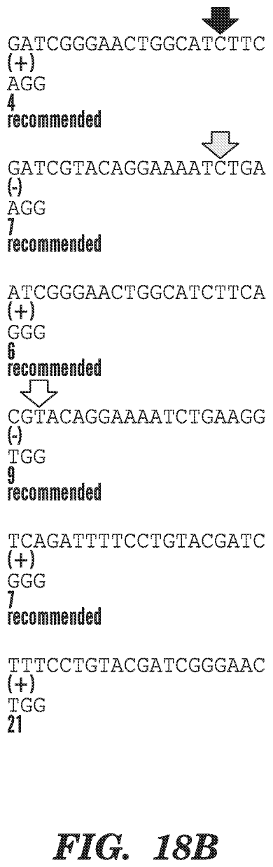

Transgene: The genomic sequence coding for the human Apo A-I gene is used to construct the transgene. Expression of the Apo A-I gene is driven by its own minimal promoter (328 bp) that has been shown to be sufficient to promote transgene expression in the liver (Walsh et al., J. Biol. Chem., 1989, 264, 6488-6494). Briefly, human Apo A-I gene is obtained by PCR on human liver genomic DNA (Clontech) and cloned in plasmid pUC19. The I-SceI site, containing two stop codons, is inserted by PCR at the beginning of a suitable exon such as exon 4 (FIG. 17 of US 20120288943 A9). The mutated gene (1-SceI-hApo A-I) is made to encode a truncated form of the native human APO A-I (80 residues vs. 267 amino-acids for the wild type APO A-I). All the constructs are sequenced and checked against the human Apo A-I gene sequence.

Generation of Transgenic Mice: An EcoRI/XbaI genomic DNA fragment carrying the mutated human Apo A-I gene is used for the generation of transgenic founders. Microinjections are done into fertilized oocytes from breeding of knock out males for the mouse apo a-I gene (WT KO mice) (The Jackson Laboratory, #002055) and B6SJLF1 females (Janvier). Transgenic founder mice (FO) are identified by PCR and Southern blot analysis on genomic DNA extracted from tail. FO are then mated to WT KO mice in order to derive I-SceI-hApo A-I transgenic lines in knock out genetic background for the endogenous murine apo a-I gene. A total of seven independent transgenic lines are studied. The molecular characterization of transgene integration is done by Southern blot experiments.

Analysis of transgene expression in each transgenic line is performed by RT-PCR on total RNA extracted from the liver (Trizol Reagent, Invitrogen). In order to avoid cross reaction with the murine transcript, primers specific for the human transgenic I-SceI-hApo A-I cDNA are used. Actin primers are used as an internal control.

Hydrodynamic-Based Transduction In vivo Transduction of transgenic mouse liver cells in vivo is performed by hydrodynamic tail vein injection. 10 to 20 g animals are injected with circular plasmid DNA in a volume of one tenth their weight in PBS in less than 10 seconds. A mixture of 20 or 50 microgram of a ceDNA coding for I-SceI under the control of the CMV promoter.

Analysis of Gene Correction:

The correction of the transgene in mice after injection of the I-SceI expression cassette and ceDNA repair matrix is analyzed by nested PCR on total liver RNA reverse transcribed using random hexamers. In order to detect the corrected gene, but not the uncorrected, primer sets that specifically amplified the repaired transgene are used. The specificity is achieved by using reverse oligonucleotides spanning the I-SceI site, forward being located outside the repair matrix Actin primers were used as an internal control.

Results:

Various transgenic lines carrying one or several copies of the I-SceI-hapo A-I transgene is used in these experiments. Mice are injected with either a mixture of I-SceI-expressing vector and ceDNA or with a vector carrying both I-SceI-expressing cassette and ceDNA. The repair of the mutated human Apo A-I gene is monitored by RT-PCR on total liver RNA using primers specifically designed to pair only with the corrected human Apo A-I gene. PCR fragments are specifically visualized in transgenic mice where I-SceI-expressing cassette and the ceDNA repair matrix were injected. The gene correction is detectable in all the transgenic lines tested containing one or several copies of the transgene.

It is shown that meganuclease-induced gene conversion can be used to perform in vivo genome surgery, and that meganucleases can be used as drugs for such applications. The ceDNA vector includes a template nucleotide sequence used as a correcting DNA strand to be inserted after a double-strand break provided by a meganuclease.

### Example 9: In Vitro AAV Transduction of Primary Human Hepatocytes

Cell culture dishes (48-well; CM1048; Lifetech) can be purchased precoated or plates (3548; VWR) can be coated with a mixture of 250 mL BDMatrigel (BD Biosciences) in 10 mL hepatocyte basal medium (CC-3199; Lonza) at 150 mL per well. Plates are incubated for 1 hour at 37°. Thawing/plating media is prepared by combining 18 mL InVitroGRO CP medium (BioreclamationIVT) and 400 mL Torpedo antibiotic mix (Celsis In vitro Technologies). Once the plates are prepared, the plateable human hepatocytes (lot #AKB; cat #F00995-P) are transferred from the liquid nitrogen vapor phase directly into the 37° water bath. The vial is stirred gently until the cells are completely thawed. The cells are transferred directly into a 50-mL conical tube containing 5 mL of prewarmed thawing/plating medium. To transfer cells completely, the vial is washed with 1 mL of thawing/plating medium. The cells are resuspended by gently swirling the tube. A small aliquot (20 mL) is removed to perform a cell count and to determine cell viability by using trypan blue solution 1:5 (25-900-C1; Cellgro). The cells are then centrifuged at 75 g for 5 minutes. The supernatant is decanted completely and the cells are resuspended at 13106 cells/mL. The matrigel mixture is aspirated from the wells, and cells are seeded at 23105 cells per well in a 48-well dish. Cells are then incubated in a 5% CO2 incubator at 37° C. At the time of transduction, cells are switched to hepatocyte culture medium (HCM) for maintenance (hepatocyte basal medium, CC-3199, Lonza; HCM, CC-4182, SingleQuots). ceDNA vector as described herein and mAlb ZFN messenger RNA (mRNA) [or in experiments replace with Cas9 mRNA and mAlb gRNA or TALEN or MN each targeted to same site as ZFN messenger; or in experiments consolidate expressed elements on ceDNA] are transfected with Lipofectamine RNAiMAX™ (Lifetech) (or other suitable reagents as disclosed herein). After 24 hours, the medium is replaced by fresh HCM, which is done daily to ensure maximal health of the primary hepatocyte cultures. For experiments in which hFIX detection by ELISA is required, sometimes the medium is not exchanged for several days to allow hFIX to accumulate in the supernatants.

### Example 10: ceDNA Vectors for In Vivo Hemophilia Treatment Using a ZFN System

ceDNA vectors comprising zinc finger nuclease-based gene editing systems can also be constructed. For illustrative purposes, an exemplary gene editing ceDNA vector is described with respect to generating a ceDNA vector encoding a zinc finger nuclease (ZFN) as the nuclease transgene, and is described below. However, while ZFN is exemplified in this Example to illustrate methods to generate a gene editing ceDNA encoding a nuclease useful in the methods and constructs as described herein, one of ordinary skill in the art is aware that one can, as stated above use, use any nuclease described herein, for example and not limited to zinc finger nucleases (ZFNs), TAL effector nucleases (TALENs), meganucleases, and CRISPR/Cas9-enzymes and engineered site-specific derivative nucleases. Exemplary nucleases to be encoded by the ceDNA vector are described herein, for example, in the sections entitled “DNA endonucleases” and the subsections therein.

Drawing on the methods described in the foregoing examples, the nuclease to be included as a transgene in the ceDNA vector can be a zinc finger nuclease (ZFN). As one nonlimiting example, the ZFN-mediated targeting of therapeutic transgenes to the albumin locus described by Sharma et al. (Blood 126: 1777-1784 (2015) may be effected using the ceDNA vectors of the invention. Such ceDNA vectors permit integration of human Factor VIII and/or Factor IX at the albumin locus in the target subject through the activity of the ceDNA-encoded ZFN targeting that locus. The ceDNA vectors may be administered to patients using any of the delivery methods described herein. Long-term expression of, e.g., human factors VIII and IX (hFVIII and hFIX) in mouse models of hemophilia A and B at therapeutic levels is achieved using this method.

### Example 11: ceDNA Vectors for In Vivo Cystic Fibrosis Treatment Using a ZFN System

An analogous approach to the experiments of Example 10 is applied to induce gene editing in the lung, for example in a subject with Cystic Fibrosis (CF). In this experiment, the ceDNA is created to encode wild-type CFTR, the gene that is mutated in CF. CFTR is a large gene that cannot be comprised within AAV. ceDNA accommodates significantly larger nucleic acid inserts than AAV, and thus provides a unique solution to the treatment of CFTR. The ceDNA vector encoding the ZFN CFTR-specific gene editing system can be administered intravenously or as a nebulized formulation to induce gene editing of lung epithelia. As above, in experiments CFTR is inserted into the endogenous CFTR locus through the activity of the encoded ZFN targeted to that locus and packaging of the nuclease and guide RNA and utilizing large homology arms may increase the efficiency and fidelity of gene editing.

### Example 12: ceDNA Vectors for In Vivo Duchenne Muscular Dystrophy Treatment

An analogous approach to the experiments of Examples 10 and 11 is applied to induce ZFN-mediated gene editing in muscle tissue, for example in Duchenne Muscular Dystrophy by correcting mutations in the dystrophin gene.

Alternatively, ceDNA vectors are created to encode endonucleases (e.g. ZNFs or TALES) that create at least two nicks and/or DSBs flanking the exon 51 splice acceptor of the dystrophin gene. Repair of these nicks and/or breaks results in deletion of the exon 51. Deletion of the exon 51 results in exclusion of exon 51 from dystrophin transcripts and thereby corrects certain DMD-causing mutations, e.g., deletion of exons 48-50. The large payload capacity of the ceDNA vectors described herein permits two endonucleases (e.g., two ZFNs as described in Ousterout et al. Molecular Therapy 2015 doi:10.1038/mt.2014.234; which is incorporated by reference herein in its entirety) or an RNA-guided endonuclease and multiple sgRNAs to be delivered to a muscle cell in a single vector, providing increased efficiency. ceDNA vectors can be administered intravenously or intramuscularly to induce gene editing of muscle tissue.

In addition, ceDNA gene editing vectors that express just one guide RNA target sequence (e.g. at one or multiple copy numbers), and/or a CRISPR/Cas nuclease (in Cis or Trans) can be used to target an individual splice donor or splice acceptor site in the DMD gene. This results in NHEJ that causes exon skipping (e.g. exon 51 skipping) and correction of the gene to express functional protein. Multiple guide RNAs that target the DMD gene are found in US. 2016/0201089, herein incorporated by reference in its entirety, see for example, Examples 5-11 therein. Correction of dystrophin expression can be tested in a DMD myoblast cell line.

### Example 13: ceDNA Gene Editing Vectors for Long-Term Therapeutic Expression from a Genomic Safe Harbor Gene

The ceDNA gene editing vectors comprising homology domains can be used to target genomic safe harbor genes for insertion and expression of therapeutic transgenes. ceDNA vectors are made according to Example 1. Any safe harbor locus can be targeted, such safe harbors are, for example, known inactive introns, or alternatively are active introns close to coding sequences known to express proteins at a high expression level. Insertion into a safe harbor gene does not have a significant negative impact as compared to absence of insertion. For example, serum albumin is a prototypical target of interest because of its high expression level and presence in liver cells. Integration of a promoter-less cassette that bears a splice acceptor site and a transgene into intronic sequences of albumin will support expression and secretion of many different proteins because albumin's first exon encodes a secretory peptide that is cleaved from the final protein product. At least one ceDNA vector encodes a Zinc Finger pair that targets intron 1. Exemplary zinc finger pairs as are described fully in Blood (2015) 126 (15): 1777-1784 (e.g., supplemental FIG. 6 pairs A-B and C-D), which is incorporated herein by reference in its entirety. Further, because of a ceDNA vector's lack of restriction, the ceDNA vector is engineered to provide the donor DNA on the same, or on a different ceDNA vector.

For illustrative purposes, an exemplary gene editing ceDNA vector is described with respect to generating a ceDNA vector comprising homology arms (also referred to as homology domains) that target the albumin safe harbor, and is described below. However, while a gene editing ceDNA with homology arms for targeted insertion of a transgene (or donor DNA) into intron 1 of albumin is exemplified in this Example to illustrate methods to generate a gene editing ceDNA with homology arms for targeted insertion of a transgene (or donor DNA) useful in the methods and constructs as described herein, one of ordinary skill in the art is aware that one can, as stated above use, use homology arms for any gene, including but not limited to safe harbor genes of locus can be used, e.g., the CCR5 or AAV-safe-harbor-S1 locus can be targeted.

FIG. 16 shows a schematic diagram depicting several promoter-less constructs for integration of donor DNA into target albumin intronic sequences, such constructs will be on the same or different vector as the nuclease. In one embodiment, the promoterless ceDNA construct comprises an insertion/repair sequence flanked by terminal repeats (e.g., ITRs) and the nuclease/guide RNA is provided using a separate construct (e.g., a second ceDNA vector, mRNA encoding a nuclease, recombinant nucleases, RNP complex etc.). A ceDNA encoding any transgene, e.g., FVIII or factor IX, or GFP or GFP and neo, without a promoter (promoter-less), is made with genomic homology arms to the albumin locus (see Example 4). In some experiments, instead of ZFN, a Cas9 or cpfl nuclease is engineered into a ceDNA vector and guide RNAs designed to target the ZFN regions. In some experiments the same ceDNA will be further engineered to express guide RNAs (see e.g., FIGS. 14, 15, and 16), and when a CAS or cpfl enzyme is used CRISPR can be provided, either on the same or different ceDNA, or on a plasmid. The guide RNAs are engineered to bind, e.g., the ZFN target sequences in Sharma et al. Blood (2015) 126 (15): 1777-1784 (e.g., supplemental FIG. 6 pairs A-B and C-D). by aligning the target sequence and identifying the PAM motif relevant to the CAS enzyme (e.g., saCAS9, or sp CAS 9, or cpfl etc.) being used. One ceDNA target center in albumin for guide RNA is shown in FIG. 17. An analogous site is used for human albumin.

The ceDNAs gene editing system for the exemplified insertion into the albumin gene to express an exemplified transgene (for example a secreted protein, e.g., Factor IX), is tested in vitro in primary human hepatocytes (e.g., human hepatocytes from Thermo Scientific) when using guide RNAs directed to human target genes, and in a mouse model to test in vivo. For example, mouse liver is isolated after systemic administration of the ceDNA system for measurement of Factor IX mRNA levels and measurement of factor IX activity using chromogenic assays and antigens, as described in Sharma et al. supra. In systems incorporating Factor IX, art-known and commercially available tests of mRNA levels, protein activity assays, and western blots are suitable for the assessment of knock-ins for any desired transgene, and to test correction in both in vitro human primary cells and in vivo mouse models. Insertion into the albumin locus allows for secretion of secreted proteins, e.g., into the blood. Plasma levels of transgene will be assessed. Secretion of human Factor VIII and Factor IX will be tested in vivo in animal models for hemophilia. It will be understood by one of ordinary skill in the art that any secreted protein can be ‘knocked into’ albumin using the ceDNAs described herein. Non-limiting examples include, a-galactosidase, iduranate-2-sulfatase, beta-glucosidase, α-L-iduronidase, etc., and can be tested in appropriate animal models. In one embodiment, the knock-ins are under control of an inducible promoter, such as Gall.

It is further contemplated herein that the torsional constraint of a ceDNA vector is released via a nCas9 nickase in combination with a guide RNA targeting the ceDNA vector itself. Such a release in torsional constraint can improve the ability of one or more homology arms in an HDR template found on the ceDNA vector. In addition, it is further contemplated herein that a guide RNA targeting the ITRs can be used with Cas9 in combination with a guide RNA for the chromosome. A single guide RNA may be designed that targets both the ITR (or homology) region of the ceDNA vector as well as the target site on the chromosome. The cut site within the ceDNA vector may be located on one or both ends of the DNA vector.

### Example 14: Exemplary Target Genes and sgRNA for Use in ceDNA Vectors

For illustrative purposes, an exemplary gene editing ceDNA vector is described with respect to generating a ceDNA vector comprising sgDNA for ZNF nucleases for editing, and is described below. However, while sgRNA sequences for ZNF nucleases to edit genes are exemplified in this Example to illustrate methods to generate a gene editing ceDNA vector useful in the methods and constructs as described herein, one of ordinary skill in the art is aware that one can, as stated above use, use sgRNAs of any nuclease as described herein, including sgRNAs for zinc finger nucleases (ZFNs), TAL effector nucleases (TALENs), meganucleases, and CRISPR/Cas9 and engineered site-specific nucleases, as discussed in the section herein entitled “DNA endonucleases” and the subsections therein.

Non-limiting exemplary target genes and target sequence pairs for ZNFs are found in Table 9; as well as gRNAs sequences based off the ZNFs target sequence. The ceDNA vectors are engineered to express ZNFs that target these sequences for correction and/or modulation of the target gene. The ceDNA vectors are engineered to express such exemplary gRNAs for correction of a target gene using e.g. any CRISPR/Cas system. Accordingly, in certain embodiments, the ceDNA vector targets a gene selected from Table 9 or Table 10. In certain embodiments, the ceDNA vector comprises a guide RNA selected from Table 9. In certain embodiments, the ceDNA vector comprises gene that encodes a ZFN that targets a target sequence selected from the following Table 9.

TABLE 9
sgRNAs to target ZFN target sequences

| | | Seq | Sequence | Seq |
|---|---|---|---|---|
| Target | Target sequence | ID | encoding | ID |
| gene | For ZFN | NO: | sgRNA | NO: |
| Human β | GGGCAGTAACGGCAGA | 608 | GTCTGCCGTT | 756 |
| globin | CTTCTCCTCAGG | | ACTGCCCTGT | |
| | | | GGG | |
| Human β | TGGGGCAAGGTGAACG | 609 | GTCTGCCGTT | 756 |
| globin | TGGATGAAGTTG | | ACTGCCCTGT | |
| | | | GGG | |
| Human β | AGAGTCAGGTGCACCA | 610 | GTAACGGCAG | 757 |
| globin | TGGTGTCTGTTT | | ACTTCaCCTC | |
| | | | AGG | |
| Human β | GTGGAGAAGTCTGCCGT | 611 | GTAACGGCAG | 757 |
| globin | TACTGCCCTGT | | ACTTCaCCTC | |
| | | | AGG | |
| Human β | ACAGGAGTCAGGTGCA | 612 | GTAACGGCAG | 757 |
| globin | CCATGGTGTCTG | | ACTTCaCCTC | |
| | | | AGG | |
| Human β | GAGAAGTCTGCCGTTAC | 613 | GTAACGGCAG | 757 |
| globin | TGCCCTGTGGG | | ACTTCaCCTC | |
| | | | AGG | |
| Human β | TAACGGCAGACTTCTCC | 614 | GTAACGGCAG | 757 |
| globin | ACAGGAGTCAG | | ACTTCaCCTC | |
| | | | AGG | |
| Human β | GCCCTGTGGGGCAAGG | 615 | GTAACGGCAG | 757 |
| globin | TGAACGTGGATG | | ACTTCaCCTC | |
| | | | AGG | |
| Human β | GGGCAGTAACGGCAGA | 608 | GTAACGGCAG | 757 |
| globin | CTTCTCCTCAGG | | ACTTCaCCTC | |
| | | | AGG | |
| Human β | TGGGGCAAGGTGAACG | 609 | GTAACGGCAG | 757 |
| globin | TGGATGAAGTTG | | ACTTCaCCTC | |
| | | | AGG | |
| Human β | CACAGGGCAGTAACGG | 616 | GTAACGGCAG | 757 |
| globin | CAGACTTCTCCT | | ACTTCaCCTC | |
| | | | AGG | |
| Human β | GGCAAGGTGAACGTGG | 617 | GTAACGGCAG | 757 |
| globin | ATGAAGTTGGTG | | ACTTCaCCTC | |
| | | | AGG | |
| Human | ATCCCATGGAGAGGTG | 618 | GCAATATGA | 758 |
| BCL11A | GCTGGGAAGGAC | | ATCCCATGGA | |
| | | | GAGG | |
| Human | ATATTGCAGACAATAAC | 619 | GCAATATGAA | 758 |
| BCL11A | CCCTTTAACCT | | TCCCATGGAG | |
| | | | AGG | |
| Human | CATCCCAGGCGTGGGG | 620 | GCATATTCTG | 759 |
| BCL11A | ATTAGAGCTCCA | | CACTCATCCC | |
| | | | AGG | |
| Human | GTGCAGAATATGCCCCG | 621 | GCATATTCTG | 759 |
| BCL11A | CAGGGTATTTG | | CACTCATCCC | |
| | | | AGG | |
| Human | GGGAAGGGGCCCAGGG | 622 | GGGCCCCTTC | 760 |
| KLF1 | CGGTCAGTGTGC | | CCGGACACAC | |
| | | | AGG | |
| Human | ACACACAGGATGACTTC | 623 | GGGCCCCTTC | 760 |
| KLF1 | CTCAAGGTGGG | | CCGGACACAC | |
| | | | AGG | |
| Human | CGCCACCGGGCTCCGG | 624 | GCAGGTCTGG | 761 |
| KLF1 | GCCCGAGAAGTT | | GGCGCGCCAC | |
| | | | CGG | |
| Human | CCCCAGACCTGCGCTCT | 623 | GCAGGTCTGG | 761 |
| KLF1 | GGCGCCCAGCG | | GGCGCGCCAC | |
| | | | CGG | |
| Human | GGCTCGGGGGCCGGGG | 626 | GGCCCCCGAG | 762 |
| KLF1 | CTGGAGCCAGGG | | CCCAAGGCGC | |
| | | | TGG | |
| Human | AAGGCGCTGGCGCTGC | 627 | GGCCCCCGAG | 762 |
| KLF1 | AACCGGTGTACC | | CCCAAGGCGC | |
| | | | TGG | |
| Human | TTGCAGCGCCAGCGCCT | 628 | GCGCTGCAAC | 763 |
| KLF1 | TGGGCTCGGGG | | CGGTGTACCC | |
| | | | GGG | |
| Human | CGGTGTACCCGGGGCCC | 629 | GCGCTGCAAC | 763 |
| KLF1 | GGCGCCGGCTC | | CGGTGTACCC | |
| | | | GGG | |
| Human γ | TTGCATTGAGATAGTGT | 630 | GCATTGAGAT | 764 |
| regulatory | GGGGAAGGGGC | | AGTGTGGGGA | |
| | | | AGG | |
| Human γ | ATCTGTCTGAAACGGTC | 631 | GCATTGAGAT | 764 |
| regulatory | CCTGGCTAAAC | | AGTGTGGGGA | |
| | | | AGG | |
| Human γ | TTTGCATTGAGATAGTG | 632 | GCATTGAGAT | 764 |
| regulatory | TGGGGAAGGGG | | AGTGTGGGGA | |
| | | | AGG | |
| Human γ | CTGTCTGAAACGGTCCC | 633 | GCATTGAGAT | 764 |
| regulatory | TGGCTAAACTC | | AGTGTGGGGA | |
| | | | AGG | |
| Human γ | TATTTGCATTGAGATAG | 634 | GCATTGAGAT | 764 |
| regulatory | TGTGGGGAAGG | | AGTGTGGGGA | |
| | | | AGG | |
| Human γ | CTGTCTGAAACGGTCCC | 633 | GCATTGAGAT | 764 |
| regulatory | TGGCTAAACTC | | AGTGTGGGGA | |
| | | | AGG | |
| | CTTGACAAGGCAAAC | 635 | GCTATTGGTC | 765 |
| | | | AAGGCAAGGC | |
| | | | TGG | |
| | GTCAAGGCAAGGCTG | 636 | GCTATTGGTC | 765 |
| | | | AAGGCAAGGC | |
| | | | TGG | |
| Human CCR5 | GATGAGGATGAC | 637 | GTGTTCATCT | 766 |
| | | | TTGGTTTTGT | |
| | | | GGG | |
| Human CCR5 | GATGAGGATGAC | 637 | GTGTTCATCT | 766 |
| | | | TTGGTTTTGT | |
| | | | GGG | |
| Human CCR5 | GATGAGGATGAC | 637 | GTGTTCATCT | 766 |
| | | | TTGGTTTTGT | |
| | | | GGG | |
| Human CCR5 | GATGAGGATGAC | 637 | GTGTTCATCT | 766 |
| | | | TTGGTTTTGT | |
| | | | GGG | |
| Human CCR5 | GATGAGGATGAC | 637 | GTGTTCATCT | 766 |
| | | | TTGGTTTTGT | |
| | | | GGG | |
| Human CCR5 | GATGAGGATGAC | 637 | GTGTTCATCT | 766 |
| | | | TTGGTTTTGT | |
| | | | GGG | |
| Human CCR5 | GATGAGGATGAC | 637 | GTGTTCATCT | 766 |
| | | | TTGGTTTTGT | |
| | | | GGG | |
| Human CCR5 | AAACTGCAAAAG | 638 | GTGTTCATCT | 766 |
| | | | TTGGTTTTGT | |
| | | | GGG | |
| Human CCR5 | AAACTGCAAAAG | 638 | GTGTTCATCT | 766 |
| | | | TTGGTTTTGT | |
| | | | GGG | |
| Human CCR5 | AAACTGCAAAAG | 638 | GTGTTCATCT | 766 |
| | | | TTGGTTTTGT | |
| | | | GGG | |
| Human CCR5 | AAACTGCAAAAG | 638 | GTGTTCATCT | 766 |
| | | | TTGGTTTTGT | |
| | | | GGG | |
| Human CCR5 | AAACTGCAAAAG | 638 | GTGTTCATCT | 766 |
| | | | TTGGTTTTGT | |
| | | | GGG | |
| Human CCR5 | AAACTGCAAAAG | 638 | GTGTTCATCT | 766 |
| | | | TTGGTTTTGT | |
| | | | GGG | |
| Human CCR5 | AAACTGCAAAAG | 638 | GTGTTCATCT | 766 |
| | | | TTGGTTTTGT | |
| | | | GGG | |
| Human CCR5 | GACAAGCAGCGG | 639 | GGTCCTGCCG | 767 |
| | | | CTGCTTGTCA | |
| | | | TGG | |
| Human CCR5 | CATCTGCTACTCG | 640 | GGTCCTGCCG | 767 |
| | | | CTGCTTGTCA | |
| | | | TGG | |
| Human CXCR4 | ATGACTTGTGGGTGGTT | 641 | GCTTCTACCC | 768 |
| | GTGTTCCAGTT | | CAATGACTTG | |
| | | | TGG | |
| Human CXCR4 | GGGTAGAAGCGGTCAC | 642 | GCTTCTACCC | 768 |
| | AGATATATCTGT | | CAATGACTTG | |
| | | | TGG | |
| Human CXCR4 | AGTCAGAGGCCAAGGA | 643 | GCCTCTGACT | 769 |
| | AGCTGTTGGCTG | | GTTGGTGGCG | |
| | | | TGG | |
| Human CXCR4 | TTGGTGGCGTGGACGAT | 644 | GCCTCTGACT | 769 |
| | GGCCAGGTAGC | | GTTGGTGGCG | |
| | | | TGG | |
| Human CXCR4 | CAGTTGATGCCGTGGCA | 645 | GCCGTGGCAA | 770 |
| | AACTGGTACTT | | ACTGGTACTT | |
| | | | TGG | |
| Human CXCR4 | CCAGAAGGGAAGCGTG | 646 | GCCGTGGCAA | 770 |
| | ATGACAAAGAGG | | ACTGGTACTT | |
| | | | TGG | |
| PPP1R12C | ACTAGGGACAGGATTG | 647 | GGGGCCACTA | 771 |
| | | | GGGACAGGAT | |
| | | | TGG | |
| PPP1R12C | CCCCACTGTGGGGTGG | 648 | GGGGCCACTA | 771 |
| | | | GGGACAGGAT | |
| | | | TGG | |
| PPP1R12C | ACTAGGGACAGGATTG | 647 | GTCACCAATC | 772 |
| | | | CTGTCCCTAG | |
| | | | TGG | |
| PPP1R12C | CCCCACTGTGGGGTGG | 648 | GTCACCAATC | 772 |
| | | | CTGTCCCTAG | |
| | | | TGG | |
| PPP1R12C | ACTAGGGACAGGATTG | 647 | GTGGCCCCAC | 773 |
| | | | TGTGGGGTGG | |
| | | | AGG | |
| PPP1R12C | CCCCACTGTGGGGTGG | 648 | GTGGCCCCAC | 773 |
| | | | TGTGGGGTGG | |
| | | | AGG | |
| Mouse | ACCCGCAGTCCCAGCGT | 649 | GTCGGCATGA | 774 |
| and | CGTGGTGAGCC | | CGGGACCGGT | |
| Human | | | CGG | |
| HPRT | | | | |
| Mouse | GCATGACGGGACCGGT | 650 | GTCGGCATGA | 774 |
| and | CGGCTCGCGGCA | | CGGGACCGGT | |
| Human | | | CGG | |
| HPRT | | | | |
| Mouse | TGATGAAGGAGATGGG | 651 | GATGTGATGA | 775 |
| and | AGGCCATCACAT | | AGGAGATGGG | |
| Human | | | AGG | |
| HPRT | | | | |
| Mouse | ATCTCGAGCAAGACGTT | 652 | GATGTGATGA | 775 |
| and | CAGTCCTACAG | | AGGAGATGGG | |
| Human | | | AGG | |
| HPRT | | | | |
| Mouse | AAGCACTGAATAGAAA | 653 | GTGCTTTGAT | 776 |
| and | TAGTGATAGATC | | GTAATCCAGC | |
| Human | | | AGG | |
| HPRT | | | | |
| Mouse | ATGTAATCCAGCAGGTC | 654 | GTGCTTTGAT | 776 |
| and | AGCAAAGAATT | | GTAATCCAGC | |
| Human | | | AGG | |
| HPRT | | | | |
| Mouse | GGCCGGCGCGCGGGCT | 655 | GTCGCCATAA | 777 |
| and | GACTGCTCAGGA | | CGGAGCCGGC | |
| Human | | | CGG | |
| HPRT | | | | |
| Mouse | GCTCCGTTATGGCGACC | 656 | GTCGCCATAA | 777 |
| and | CGCAGCCCTGG | | CGGAGCCGGC | |
| Human | | | CGG | |
| HPRT | | | | |
| Mouse | TGCAAAAGGTAGGAAA | 657 | GTATTGCAAA | 778 |
| and | AGGACCAACCAG | | AGGTAGGAAA | |
| Human | | | AGG | |
| HPRT | | | | |
| Mouse | ACCCAGATACAAACAA | 658 | GTATTGCAAA | 778 |
| and | TGGATAGAAAAC | | AGGTAGGAAA | |
| Human | | | AGG | |
| HPRT | | | | |
| Mouse | CTGGGATGAACTCTGGG | 659 | GCATATCTGG | 779 |
| and | CAGAATTCACA | | GATGAACTCT | |
| Human | | | GGG | |
| HPRT | | | | |
| Mouse | ATGCAGTCTAAGAATAC | 660 | GCATATCTGG | 779 |
| and | AGACAGATCAG | | GATGAACTCT | |
| Human | | | GGG | |
| HPRT | | | | |
| Mouse | TGCACAGGGGCTGAAG | 661 | GCCTCCTGGC | 780 |
| and | TTGTCCCACAGG | | CATGTGCACA | |
| Human | | | GGG | |
| HPRT | | | | |
| Mouse | TGGCCAGGAGGCTGGTT | 662 | GCCTCCTGGC | 780 |
| and | GCAAACATTTT | | CATGTGCACA | |
| Human | | | GGG | |
| HPRT | | | | |
| Mouse | TTGAATGTGATTTGAAA | 663 | GAAGCTGATG | 781 |
| and | GGTAATTTAGT | | ATTTAAGCTT | |
| Human | | | TGG | |
| HPRT | | | | |
| Mouse | AAGCTGATGATTTAAGC | 664 | GAAGCTGATG | 781 |
| and | TTTGGCGGTTT | | ATTTAAGCTT | |
| Human | | | TGG | |
| HPRT | | | | |
| Mouse | GTGGGGTAATTGATCCA | 665 | GATCAATTAC | 782 |
| and | TGTATGCCATT | | CCCACCTGGG | |
| Human | | | TGG | |
| HPRT | | | | |
| Mouse | GGGTGGCCAAAGGAAC | 666 | GATCAATTAC | 782 |
| and | TGCGCGAACCTC | | CCCACCTGGG | |
| Human | | | TGG | |
| HPRT | | | | |
| Mouse | ATCAACTGGAGTTGGAC | 667 | GATGTCTTTA | 783 |
| and | TGTAATACCAG | | CAGAGACAAG | |
| Human | | | AGG | |
| HPRT | | | | |
| Mouse | CTTTACAGAGACAAGA | 668 | GATGTCTTTA | 783 |
| and | GGAATAAAGGAA | | CAGAGACAAG | |
| Human | | | AGG | |
| HPRT | | | | |
| Human | CCTATCCATTGCACTAT | 669 | GATCAACAGC | 784 |
| albumin | GCTTTATTTAA | | ACAGGTTTT | |
| | | | GTGG | |
| Human | CCTATCCATTGCACTAT | 669 | GATCAACAGC | 784 |
| albumin | GCTTTATTTAA | | ACAGGTTTTG | |
| | | | TGG | |
| Human | CCTATCCATTGCACTAT | 669 | GATCAACAGC | 784 |
| albumin | GCTTTATTTAA | | ACAGGTTTTG | |
| | | | TGG | |
| Human | CCTATCCATTGCACTAT | 669 | GATCAACAGC | 784 |
| albumin | GCTTTATTTAA | | ACAGGTTTTG | |
| | | | TGG | |
| Human | CCTATCCATTGCACTAT | 669 | GATCAACAGC | 784 |
| albumin | GCTTTATTTAA | | ACAGGTTTTG | |
| | | | TGG | |
| Human | CCTATCCATTGCACTAT | 669 | GATCAACAGC | 784 |
| albumin | GCTTTATTTAA | | ACAGGTTTTG | |
| | | | TGG | |
| Human | TTTGGGATAGTTATGAA | 670 | GATCAACAGC | 784 |
| albumin | TTCAATCTTCA | | ACAGGTTTTG | |
| | | | TGG | |
| Human | TTTGGGATAGTTATGAA | 670 | GATCAACAGC | 784 |
| albumin | TTCAATCTTCA | | ACAGGTTTTG | |
| | | | TGG | |
| Human | TTTGGGATAGTTATGAA | 670 | GATCAACAGC | 784 |
| albumin | TTCAATCTTCA | | ACAGGTTTTG | |
| | | | TGG | |
| Human | TTTGGGATAGTTATGAA | 670 | GATCAACAGC | 784 |
| albumin | TTCAATCTTCA | | ACAGGTTTTG | |
| | | | TGG | |
| Human | CCTGTGCTGTTGATCTC | 671 | GATCAACAGC | 784 |
| albumin | ATAAATAGAAC | | ACAGGTTTTG | |
| | | | TGG | |
| Human | CCTGTGCTGTTGATCTC | 671 | GATCAACAGC | 784 |
| albumin | ATAAATAGAAC | | ACAGGTTTTG | |
| | | | TGG | |
| Human | TTGTGGTTTTTAAATAA | 672 | GATCAACAGC | 784 |
| albumin | AGCATAGTGCA | | ACAGGTTTTG | |
| | | | TGG | |
| Human | TTGTGGTTTTTAAATAA | 672 | GATCAACAGC | 784 |
| albumin | AGCATAGTGCA | | ACAGGTTTTG | |
| | | | TGG | |
| Human | ACCAAGAAGACAGACT | 673 | GATCAACAGC | 784 |
| albumin | AAAATGAAAATA | | ACAGGTTTTG | |
| | | | TGG | |
| Human | CTGTTGATAGACACTAA | 674 | GATCAACAGC | 784 |
| albumin | AAGACTATTAG | | ACAGGTTTTG | |
| | | | TGG | |
| Human | TGACACAGTACCTGGCA | 675 | GTCAGGGTAC | 785 |
| Factor IX | CCATAGTTGTA | | TAGGGGTATG | |
| | | | GGG | |
| Human | GTACTAGGGGTATGGG | 676 | GTCAGGGTAC | 785 |
| Factor IX | GATAAACCAGAC | | TAGGGGTAT | |
| | | | GGGG | |
| Human | GCAAAGATTGCTGACTA | 677 | GTCAGCAATC | 786 |
| LRRK2 | CGGCATTGCTC | | TTTGCAATGA | |
| | | | TGG | |
| Human | TGATGGCAGCATTGGG | 678 | GTCAGCAATC | 786 |
| LRRK2 | ATACAGTGTGAA | | TTTGCAATGA | |
| | | | TGG | |
| Human | GCAAAGATTGCTGACTA | 679 | GTCAGCAATC | 786 |
| LRRK2 | CAGCATTGCTC | | TTTGCAATGA | |
| | | | TGG | |
| Human Htt | GGGGCGATGCTGGGGA | 680 | | |
| | CGGGGACATTAG | | | |
| Human Htt | ACGCTGCGCCGGCGGA | 681 | GTCTGGGACG | 787 |
| | GGCGGGGCCGCG | | CAAGGCGCCG | |
| | | | TGG | |
| Human Htt | AAGGCGCCGTGGGGGC | 682 | GTCTGGGACG | 787 |
| | TGCCGGGACGGG | | CAAGGCGCCG | |
| | | | TGG | |
| Human Htt | AGTCCCCGGAGGCCTCG | 683 | GGAGGCCTCG | 788 |
| | GGCCGACTCGC | | GGCCGACTCG | |
| | | | CGG | |
| Human Htt | GCGCTCAGCAGGTGGT | 684 | GCCGGTGATA | 789 |
| | GACCTTGTGGAC | | TGGGCTTCCT | |
| | | | GGG | |
| Human Htt | ATGGTGGGAGAGACTG | 685 | GAGACTGTGA | 790 |
| | TGAGGCGGCAGC | | GGCGGCAGCT | |
| | | | GGG | |
| Human Htt | ATGGCGCTCAGCAGGT | 686 | GAGACTGTGA | 790 |
| | GGTGACCTTGTG | | GGCGGCAGCT | |
| | | | GGG | |
| Human Htt | TGGGAGAGACTGTGAG | 687 | GAGACTGTGA | 790 |
| | GCGGCAGCTGGG | | GGCGGCAGCT | |
| | | | GGG | |
| Human | GCCAGGTAGTACTGTGG | 688 | GGCTCAGCCA | 791 |
| RHO | GTACTCGAAGG | | GGTAGTACTG | |
| | | | TGG | |
| Human | GAGCCATGGCAGTTCTC | 689 | GGCTCAGCCA | 791 |
| RHO | CATGCTGGCCG | | GGTAGTACTG | |
| | | | TGG | |
| Human | CAGTGGGTTCTTGCCGC | 690 | GAACCCACTG | 792 |
| RHO | AGCAGATGGTG | | GGTGACGATG | |
| | | | AGG | |
| Human | GTGACGATGAGGCCTCT | 691 | GAACCCACTG | 792 |
| RHO | GCTACCGTGTC | | GGTGACGATG | |
| | | | AGG | |
| Human | GGGGAGACAGGGCAAG | 692 | GCCCTGTCTC | 793 |
| RHO | GCTGGCAGAGAG | | CCCCATGTCC | |
| | | | AGG | |
| Human | ATGTCCAGGCTGCTGCC | 693 | GCCCTGTCTC | 793 |
| RHO | TCGGTCCCATT | | CCCCATGTCC | |
| | | | AGG | |
| CFTR | ATTAGAAGTGAAGTCTG | 694 | GGGAGAACTG | 794 |
| | GAAATAAAACC | | GAGCCTTCAG | |
| | | | AGG | |
| CFTR | AGTGATTATGGGAGAA | 695 | GGGAGAACTG | 794 |
| | CTGGATGTTCACAGTCA | | GAGCCTTCAG | |
| | GTCCACACGTC | | AGG | |
| CFTR | CATCATAGGAAACACC | 696 | GAGGGTAAAA | 795 |
| | AAAGATGATATT | | TTAAGCACAG | |
| | | | TGG | |
| CFTR | ATATAGATACAGAAGC | 697 | GAGGGTAAAA | 795 |
| | GTCATCAAAGCA | | TTAAGCACAG | |
| | | | TGG | |
| CFTR | GCTTTGATGACGCTTCT | 698 | GAGGGTAAAA | 795 |
| | GTATCTATATT | | TTAAGCACAG | |
| | | | TGG | |
| CFTR | CCAACTAGAAGAGGTA | 699 | GAGGGTAAAA | 795 |
| | AGAAACTATGTG | | TTAAGCACAG | |
| | | | TGG | |
| CFTR | CCTATGATGAATATAGA | 700 | GAGGGTAAAA | 795 |
| | TACAGAAGCGT | | TTAAGCACAG | |
| | | | TGG | |
| CFTR | ACACCAATGATATTTTC | 701 | GAGGGTAAAA | 795 |
| | TTTAATGGTGC | | TTAAGCACAG | |
| | | | TGG | |
| TRAC | CTATGGACTTCAAGAGC | 702 | GAGAATCAAA | 796 |
| | AACAGTGCTGT | | ATCGGTGAAT | |
| | | | AGG | |
| TRAC | CTCATGTCTAGCACAGT | 703 | GAGAATCAAA | 796 |
| | TTTGTCTGTGA | | ATCGGTGAAT | |
| | | | AGG | |
| TRAC | GTGCTGTGGCCTGGAGC | 704 | GAGAATCAAA | 796 |
| | AACAAATCTGA | | ATCGGTGAAT | |
| | | | AGG | |
| TRAC | TTGCTCTTGAAGTCCAT | 705 | GAGAATCAAA | 796 |
| | AGACCTCATGT | | ATCGGTGAAT | |
| | | | AGG | |
| TRAC | GCTGTGGCCTGGAGCA | 706 | GACACCTTCT | 797 |
| | ACAAATCTGACT | | TCCCCAGCCC | |
| | | | AGG | |
| TRAC | CTGTTGCTCTTGAAGTC | 707 | GACACCTTCT | 797 |
| | CATAGACCTCA | | TCCCCAGCCC | |
| | | | AGG | |
| TRAC | CTGTGGCCTGGAGCAAC | 708 | GACACCTTCT | 797 |
| | AAATCTGACTT | | TCCCCAGCCC | |
| | | | AGG | |
| TRAC | CTGACTTTGCATGTGCA | 709 | GACACCTTCT | 797 |
| | AACGCCTTCAA | | TCCCCAGCCC | |
| | | | AGG | |
| TRAC | TTGTTGCTCCAGGCCAC | 710 | GACACCTTCT | 797 |
| | AGCACTGTTGC | | TCCCCAGCCC | |
| | | | AGG | |
| TRAC | TGAAAGTGGCCGGGTTT | 711 | GACACCTTCT | 797 |
| | AATCTGCTCAT | | TCCCCAGCCC | |
| | | | AGG | |
| TRAC | AGGAGGATTCGGAACC | 712 | GATTAAACCC | 798 |
| | CAATCACTGACA | | GGCCACTTTC | |
| | | | AGG | |
| TRAC | GAGGAGGATTCCiGAAC | 713 | GATTAAACCC | 798 |
| | CCAATCACTGAC | | GGCCACTTTC | |
| | | | AGG | |
| TRAC | TGAAAGTGGCCGGGTTT | 711 | GATTAAACCC | 798 |
| | AATCTGCTCAT | | GGCCACTTTC | |
| | | | AGG | |
| TRBC | CCGTAGAACTGGACTTG | 714 | GCTGTCAAGT | 799 |
| | ACAGCGGAAGT | | CCAGTTCTAC | |
| | | | GGG | |
| TRBC | TCTCGGAGAATGACGA | 715 | GCTGTCAAGT | 799 |
| | GTGGACCCAGGA | | CCAGTTCTAC | |
| | | | GGG | |
| TRBC | TCTCGGAGAATGACGA | 715 | GCTGTCAAGT | 799 |
| | GTGGACCCAGGA | | CCAGTTCTAC | |
| | | | GGG | |
| TRBC | TCTCGGAGAATGACGA | 715 | GCTGTCAAGT | 799 |
| | GTGGACCCAGGA | | CCAGTTCTAC | |
| | | | GGG | |
| TRBC | TCTCGGAGAATGACGA | 715 | GCTGTCAAGT | 799 |
| | GTGGACCCAGGA | | CCAGTTCTAC | |
| | | | GGG | |
| TRBC | CCGTAGAACTGGACTTG | 714 | GCTGTCAAGT | 799 |
| | ACAGCGGAAGT | | CCAGTTCTAC | |
| | | | GGG | |
| TRBC | CCGTAGAACTGGACTTG | 714 | GCTGTCAAGT | 799 |
| | ACAGCGGAAGT | | CCAGTTCTAC | |
| | | | GGG | |
| TRBC | CCGTAGAACTGGACTTG | 714 | GCTGTCAAGT | 799 |
| | ACAGCGGAAGT | | CCAGTTCTAC | |
| | | | GGG | |
| TRBC | CCGTAGAACTGGACTTG | 714 | GCTGTCAAGT | 799 |
| | ACAGCGGAAGT | | CCAGTTCTAC | |
| | | | GGG | |
| Human | CCAGGGCGCCTGTGGG | 716 | GGCGCCCTGG | 800 |
| PD1 | ATCTGCATGCCT | | CCAGTCGTCT | |
| | | | GGG | |
| Human | CAGTCGTCTGGGCGGTG | 717 | GGCGCCCTGG | 800 |
| PD1 | CTACAACTGGG | | CCAGTCGTCT | |
| | | | GGG | |
| Human | GAACACAGGCACGGCT | 718 | GTCCACAGAG | 801 |
| PD1 | GAGGGGTCCTCC | | AACACAGGCA | |
| | | | CGG | |
| Human | CTGTGGACTATGGGGA | 719 | GTCCACAGAG | 801 |
| PD1 | GCTGGATTTCCA | | AACACAGGCA | |
| | | | CGG | |
| Human | CAGTCGTCTGGGCGGTG | 720 | GGCGCCCTGG | 800 |
| PD1 | CT | | CCAGTCGTCT | |
| | | | GGG | |
| Human | ACAGTGCTTCGGCAGGC | 721 | GCTTCGGCAG | 802 |
| CTLA-4 | TGACAGCCAGG | | GCTGACAGCC | |
| | | | AGG | |
| Human | ACCCGGACCTCAGTGGC | 722 | GCTTCGGCAG | 802 |
| CTLA-4 | TTTGCCTGGAG | | GCTGACAGCC | |
| | | | AGG | |
| Human | ACTACCTGGGCATAGGC | 723 | GTACCCACC | 803 |
| CTLA-4 | AACGGAACCCA | | GCCATACTAC | |
| | | | CTGG | |
| Human | TGGCGGTGGGTACATG | 724 | GTACCCACCG | 803 |
| CTLA-4 | AGCTCCACCTTG | | CCATACTACC | |
| | | | TGG | |
| HLA C11: | GTATGGCTGCGACGTGG | 725 | GCTGCGACGT | 804 |
| HLA A2 | GGTCGGACGGG | | GGGGTCGGAC | |
| | | | GGG | |
| HLA C11: | TTATCTGGATGGTGTGA | 726 | GCAGCCATAC | 805 |
| HLA A2 | GAACCTGGCCC | | ATTATCTGGA | |
| | | | TGG | |
| HLA C11: | TCCTCTGGACGGTGTGA | 727 | GCAGCCAT | 806 |
| HLA A2 | GAACCTGGCCC | | ACATCCTC | |
| | | | TGGACGG | |
| HLA A3 | ATGGAGCCGCGGGCGC | 728 | GTGGATA | 807 |
| | CCiTGGATAGAGC | | GAGCAG | |
| | | | GAGGG | |
| | | | GCCG | |
| | | | G | |
| HLA A3 | CTGGCTCGCGGCGTCGC | 729 | GAGCCAGAGG | 808 |
| | TGTCGAACCGC | | ATGGAGCCGC | |
| | | | GG | |
| | | | G | |
| HLA B | TCCAGGAGCTCAGGTCC | 730 | GGACCTGA | 809 |
| | TCGTTCAGGGC | | GCTCCTGGAC | |
| | | | CGCGG | |
| HLA B | CGGCGGACACCGCGGC | 731 | GGACCTGA | 809 |
| | TCAGATCACCCA | | GCTCCTGGAC | |
| | | | CGCGG | |
| HLA B | AGGTGGATGCCCAGGA | 732 | GATGCCCAGG | 810 |
| | CGAGCTTTGAGG | | ACGAGCTTTG | |
| | | | AGG | |
| HLA B | AGGGAGCAGAAGCAGC | 733 | GCGCTGCTTC | 811 |
| | GCAGCAGCGCCA | | TGCTCCCTGG | |
| | | | AGG | |
| HLA B | CTGGAGGTGGATGCCC | 734 | GCGCTGCTTC | 811 |
| | AGGACGAGCTTT | | TGCTCCCTGG | |
| | | | AGG | |
| HLA B | GAGCAGAAGCAGCGCA | 735 | GCGCTGCTTC | 811 |
| | GCAGCGCCACCT | | TGCTCCCTGG | |
| | | | AGG | |
| HLA C | CCTCAGTTTCATGGGGA | 736 | GGGGATTCAA | 812 |
| | TTCAAGGGAAC | | GGGAACACCC | |
| | | | TGG | |
| HLA C | CCTAGGAGGTCATGGG | 737 | GCAAATGCCC | 813 |
| | CATTTGCCATGC | | ATGACCTCCT | |
| | | | AGG | |
| HLA C | TCGCGGCGTCGCTGTCG | 738 | GAGCCAGAGG | 808 |
| | AACCGCACGAA | | ATGGAGCCGC | |
| | | | GG | |
| | | | G | |
| HLA C | CCAAGAGGGGAGCCGC | 739 | GGCGCCCGCG | 814 |
| | GGGAGCCGTGGG | | GCTCCCCTCT | |
| | | | TGG | |
| HLA | GAAATAAGGCATACTG | 740 | GTTCACATCT | 815 |
| cl.II: | GTATTACTAATG | | CCCCCGGGCC | |
| DBP2 | | | TGG | |
| HLA | GAGGAGAGCAGGCCGA | 741 | GTTCACATCT | 815 |
| cl.II: | TTACCTGACCCA | | CCCCCGGGCC | |
| DBP2 | | | TGG | |
| DRA | TCTCCCAGGGTGGTTCA | 742 | GGAGAATGCG | 816 |
| | GTGGCAGAATT | | GGGGAAAGAG | |
| | | | AGG | |
| DRA | GCGGGGGAAAGAGAGG | 743 | GGAGAATGCG | 816 |
| | AGGAGAGAAGGA | | GGGGAAAGAG | |
| | | | AGG | |
| TAPI | AGAAGGCTGTGGGCTC | 744 | GCCCACAGCC | 817 |
| | CTCAGAGAAAAT | | TTCTGTACTC | |
| | | | TGG | |
| TAPI | ACTCTGGGGTAGATGG | 745 | GCCCACAGCC | 817 |
| | AGAGCAGTACCT | | TTCTGTACTC | |
| | | | TGG | |
| TAP2 | TTGCGGATCCGGGAGC | 746 | GTTGATTCGA | 818 |
| | AGCTTTTCTCCT | | GACATGGTGT | |
| | | | AGG | |
| TAP2 | TTGATTCGAGACATGGT | 747 | GTTGATTCGA | 818 |
| | GTAGGTGAAGC | | GACATGGTGT | |
| | | | AGG | |
| Tapasin | CCACAGCCAGAGCCTC | 748 | GCTCTGGCTG | 819 |
| | AGCAGGAGCCTG | | TGGTCGCAAG | |
| | | | AGG | |
| Tapasin | CGCAAGAGGCTGGAGA | 749 | GCTCTGGCTG | 819 |
| | GGCTGAGGACTG | | TGGTCGCAAG | |
| | | | AGG | |
| Tapasin | CTGGATGGGGCTTGGCT | 750 | GCAGAACTGC | 820 |
| | GATGGTCAGCA | | CCGCGGGCCC | |
| | | | TGG | |
| Tapasin | GCCCGCGGGCAGTTCTG | 751 | GCAGAACTGC | 820 |
| | CGCGGGGGTCA | | CCGCGGGCCC | |
| | | | TGG | |
| CIITA | GCTCCCAGGCAGCGGG | 752 | GCTGCCTGGG | 821 |
| | CGGGAGGCTGGA | | AGCCCTACTC | |
| | | | GGG | |
| CIITA | CTACTCGGGCCATCGGC | 753 | GCTGCCTGGG | 821 |
| | GGCTGCCTCGG | | AGCCCTACTC | |
| | | | GGG | |
| RFX5 | TTGATGTCAGGGAAGAT | 754 | GCCTTCGAGC | 822 |
| | CTCTCTGATGA | | TTTGATGTCA | |
| | | | GGG | |
| RFX5 | GCTCGAAGGCTTGGTGG | 755 | GCCTTCGAGC | 822 |
| | CCGGGGCCAGT | | TTTGATGTCA | |
| | | | GGG | |

TABLE 10
Exemplary genes for targeting (see e.g., US 2015/0056705, which is
incorporated herein in its entirety by reference)

| | | Representative Accession |
|---|---|---|
| Gene name | location | (cDNA) RefSeq |
| HBB | chr11: 5246696-5248301 | (NM_000518) |
| BCL11A | chr2: 60684329-60780633 | (NM_022893) |
| KLF1 | chr19: 12995237-12998017 | (NM_006563) |
| HBG1 | chr11: 5269502-5271087 | (NM_000559) |
| CCR5 | chr3: 46411633-46417697 | (NM_000579) |
| CXCR4 | chr2: 136871919-136873813 | (NM_001008540) |
| PPP1R12C | chr19: 55602281-55628968 | (NM_017607) |
| HPRT | chrX: 133594175-133634698 | (NM_000194) |
| Mouse HPRT | chrX: 52988078-53021660 | (NM_013556) |
| | (assembly GRCm38/mm10) | |
| ALB | chr4: 74269972-74287129 | (NM_000477) |
| Factor VIII | chrX: 154064064-154250998 | (NM_000132.3) |
| Factor IX | chrX: 138612895-138645617 | (NM_000133) |
| LRRK2 | chr12: 40618813-40763086 | (NM_198578) |
| Htt | chr4: 3076237-3245687 | (NM_002111) |
| RHO | chr3: 129247482-129254187 | (NM_000539) |
| CFTR | chr7: 117120017-117308718 | (NM_000492) |
| TCRA | chr6: 42883727-42893575 | (NM_001243168) |
| TCRB | chr7: 142197572-142198055 | L36092.2 |
| PD-1 | chr2: 242792033-242795132 | (NM_005018) |
| CTLA-4 | chr2: 204732511-204738683 | (NM_001037631) |
| HLA-A | chr6: 29910247-29912868 | (NM_002116) |
| HLA-B | chr6: 31236526-31239913 | NM_005514.6 |
| HLA-C | chr6: 31236526-31239125 | (NM_001243042) |
| HLA-DPA | chr6: 33032346-33048555 | (NM_033554.3) |
| HLA-DQ | chr6: 32605183-32611429 | (NM_002122) |
| HLA-DRA | chr6_ssto_hap7: 3754283- | (NM_019111) |
| | 3759493 | |
| LMP7 | chr6_dbb_hap3: 4089872- | (X66401) |
| | 4093057 | |
| Tapasin | chr6: 33271410-33282164 | (NM_172208) |
| RFX5 | chr1: 151313116-151319769 | (NM_001025603) |
| CIITA | chr16: 10971055-11002744 | (NM_000246) |
| TAP1 | chr6: 32812986-32821748 | (NM_000593) |
| TAP2 | chr6: 32793187-32806547 | (NM_000544) |
| TAPBP | chr6: 33267472-33282164 | |
| DMD | chrX: 31137345-33229673 | (NM_004006) |
| RFX5 | chr1: 151313116-151319769 | (NM_000449) |
| B. napus FAD3 | See PCT publication | JN992612 |
| | WO2014/039684 | |
| B. napus FAD2 | See PCT publication | JN992609 |
| | WO2014/039692 | |
| Soybean FAD2 | See US20140090116 | |
| Zea mays ZP15 | See U.S. Pat. No. 8,329,986 | GBWI-61522 (MaizeCyc) |
| B-ketoacyl ACP | See U.S. Pat. No. 8,592,645 | |
| synthase II | | |
| (KASII) | | |
| Tomato MDH | See US 20130326725 | AY725474 |
| B. napus EPSPS | See U.S. Pat. No. 8, 399, 218 | |
| paralogs C + D | | |
| Paralog D | See U.S. Pat. No. 8,399,218 | |
| Paralog A + B | See U.S. Pat. No. 8,399,218 | |
| PPP1R12C | chr19: 55602840-55624858 | (NM_017607) |
| (AAVS1) | | |
| GR | 5: 142646254-142783254 | (NM_000176) |
| IL2RG | chrX: 70327254-70331481 | (NM_000206) |
| SFTPB | chr2: 85884440-85895374 | (NM_198843) |

### Example 15: ceDNA Gene Editing Vectors for Engineering of T Cells

As disclosed herein, the ceDNA gene editing vectors described herein can be used to edit, repair, and/or knock-out genes in the genome of any cell, for example, in a T cell. For illustrative purposes, an exemplary gene editing ceDNA vector is described with respect to generating a ceDNA vector for editing any of CXCR4, CCR5, PD-1 genes in T-cells and is described below. However, while targeting CXCR4, CCR5 or PD-1 genes are exemplified in this Example to illustrate methods to generate a gene editing vector ceDNA useful in the methods and constructs as described herein, one of ordinary skill in the art is aware that one can, as stated above use, use any gene where gene editing is desired, for example, as described herein in the sections entitled “Exemplary diseases to be treated with a gene editing ceDNA” and “additional diseases for gene editing”. Additionally, while the genome of T cells is modified in this illustrative example, one of ordinary skill is aware that any cell can be modified, ex vivo or in vivo, for example, any cell as described in section XII.A. herein entitled “host cells”. Also, while genomic DNA is shown in this illustrative example to be modified, it is envisioned that the ceDNA vectors can also be modified by an ordinary skilled artisan to modify mitochondrial DNA (mtDNA), e.g., to encode mtZFN and mitoTALEN function, or mitochondrial-adapted CRISPR/Cas9 platform as described in Maeder, et al. “Genome-editing technologies for gene and cell therapy.” Molecular Therapy 24.3 (2016): 430-446 and Gammage P A, et al. Mitochondrial Genome Engineering: The Revolution May Not Be CRISPR-Ized. Trends Genet. 2018; 34(2):101-110.

Any therapeutically relevant gene can be targeted (e.g., CXCR4, or CCR5, the coreceptor for HIV entry), and can be ablated, edited, repaired or replaced (in the case of CXCR4 e.g., to prevent HIV entry). In a further non-limiting example, PD-1, a mediator of T cell exhaustion, can be ablated. Ablation of target genes is performed with or without a template nucleic acid sequence, e.g. donor HDR template. Use of a single guide RNA (sgRNA) and corresponding nuclease in the absence of an HDR template results in non-homologous-end-joining (NHEJ).

Any therapeutically-relevant locus can be targeted, such targeted loci are, e.g., known regulators of T cell exhaustion, viral coreceptors, and the like. The ceDNA gene editing vectors can comprise any endonuclease as described herein, including RNA-guided endonucleases, e.g., CRISPR/Cas9 and other endonucleases including zinc finger nucleases (ZFNs), TAL effector nucleases (TALENs), meganucleases and engineered site-specific derivative nucleases as described herein, for example, in the sections entitled “DNA endonucleases” and the subsections therein. Exemplary suitable endonucleases and template nucleic acids for HDR of CXCR4 and PD-1 are described fully in Schumann et al., PNAS (2015) 112(33):10437-10442, which is incorporated herein by reference in its entirety, (See for example FIGS. 1-4 in Schumann et al.,).

Further, because of ceDNA's lack of size restriction, the ceDNA vector can be engineered to provide the donor DNA and/or gene editing molecules on the same ceDNA vector. Alternatively, the donor DNA and/or gene editing molecules can be provided on one or more different ceDNA vectors.

A ceDNA encoding template nucleic acids suitable for ablation of a target locus, e.g., disruption of the promoter or insertion of a premature stop codon or missense mutation, will be made with genomic homology arms to the target locus (see, e.g., Example 10). As such, in the modified cell, the gene will be truncated or gene silenced. In some experiments, a Cas9 or cpfl nuclease will be engineered into the same or different ceDNA vector. In some experiments the ceDNA will be further engineered to express guide RNAs (see e.g., FIGS. 12, 15, 16), and when a Cas or cpfl enzyme is used it can be provided, either on the same or different ceDNA, or by plasmid, or by mRNA, or by recombinant protein. The guide RNAs are engineered to bind, e.g., the target sequences in in PNAS (2015) 112(33):10437-10442 by aligning the target sequence and identifying the PAM motif relevant to the Cas enzyme (e.g., saCas9, or spCas9, or cpfl etc.) being used.

In some experiments, the guide RNAs will target other known sequence regions. Multiple sgRNA sequences that bind known target regions are described in Tables 1-2 of US patent publication 2015/0056705, which is herein incorporated by reference in its entirety, and include for example gRNA sequences for human beta-globin, human, BCLIIA, human KLF1, Human CCR5, Human CXCR4, PPP1R12C, mouse and human HPRT, human albumin, human factor DC, human factor VIII, human LRRK2, human Htt, human RH, CFTR, TRAC, TRBC, human PD1, human CTLA-4, HLA c11, HLA A2, HLA A3, HLA B, HLA C, HLA c1. II DBp2. DRA, Tap 1 and 2. Tapasin, DMD, RFX5, etc.,).

The ceDNA vectors will be delivered to T cells ex vivo, but systemic delivery is also contemplated herein. In some experiments, instead of a Cas9 or cpfl nuclease, a zinc finger nuclease, TALENS, or megaTALs will be engineered into the same or different ceDNA vector.

### Example 16: Ex Vivo Gene Editing to Treat Wiscott-Aldrich Syndrome (WAS)

Ex vivo gene editing using AAV is challenging in that AAV is the only source of a homology repair template or DNA donor template. AAV vectors comprise an encapsidated DNA, which limits the size of the homology arms that can be delivered. In addition, the complexity and high costs associated with AAV limit its usefulness with respect to ex vivo gene editing. In addition, AAV vectors are required at very high titers to induce sufficient homology directed repair in cells in culture.

CeDNA vectors as described herein can overcome many of the problems associated with AAV vector-mediated delivery of a DNA donor template. For example, ceDNA vectors permit the use of donor templates having longer homology arms to those used with conventional AAV vectors, which provides an advantage of enabling more efficient gene editing with higher on-target and lower off-target effects.

For illustrative purposes, an exemplary gene editing ceDNA vector is described with respect to generating a ceDNA vector for editing WAS genes CD34+ stem cells ex vivo is described below. However, while targeting of the WAS gene is exemplified in this Example to illustrate methods to generate a gene editing vector ceDNA useful in the methods and constructs as described herein, one of ordinary skill in the art is aware that one can, as stated above use, use any gene where gene editing is desired. Exemplary genes for editing are described herein, for example, in the sections entitled “Exemplary diseases to be treated with a gene editing ceDNA” and “additional diseases for gene editing”. Additionally, while the genome of hematopoetic CD34+ cells is modified in this illustrative example, one of ordinary skill is aware that any cell, including somatic cells, cultured cells as well as stem cells and/or pluripotent cells, can be modified, ex vivo or in vivo, for example, any cell as described in section XII.A. herein entitled “host cells”.

Ex vivo experiments will be performed using human CD34+ hematopoietic stem cells to test the ability of a ceDNA vector encoding the Wiscott-Aldrich Syndrome (WAS) gene open reading frame (ORF) to perform gene editing in culture. An exemplary experiment comprising five different treatment arms is outlined herein below.

First, ceDNA vectors will be used to deliver a construct currently delivered using AAV vectors and that encodes the WAS ORF (minigene; exons only) and homology regions. Efficiency of the gene editing results will be compared to the efficiency achieved using AAV (30-40%) and will determine whether a ceDNA vector-based delivery can meet or exceed the efficiency of AAV-mediated delivery of the same minigene.

Next, ceDNA vectors will be used to deliver the WAS minigene (exons only) with intron-1 retained. Intron-1 has been found to be critical for expression of the WAS protein, but Intron-1 exceeds the size limitations of AAV vectors. Thus, successful delivery of the WAS minigene+intron-1 will show that ceDNA vectors are superior to AAV vectors for gene editing, targeted delivery of the WAS minigene+Intron-1 and successful expression of the WAS protein.

It is next contemplated that ceDNA vectors comprising the WAS ORF minigene or the WAS minigene+Intron-1 will be designed with longer homology arms to assess whether the increased length of such homology arms has an impact on the on-target efficiency or the off-target fidelity of gene editing.

CeDNA vectors comprising the WAS ORF minigene or the WAS minigene+Intron-1 are next designed to comprise a Cas9 cleavage site on the ceDNA to determine if the presence of the Cas9 site enhances the efficiency of ceDNA to act as a donor template by releasing torsional tension of the ceDNA vector. Finally, ceDNA vectors encoding reporter constructs, such as ceDNA-GFP/ceDNA-LUC, can be designed to optimize and/or maximize the efficiency of electroporation of the ceDNA vectors in ex vivo cells.

### Example 17: Exemplary Work-Flow Method(s) for Gene Editing in Cultured Cells

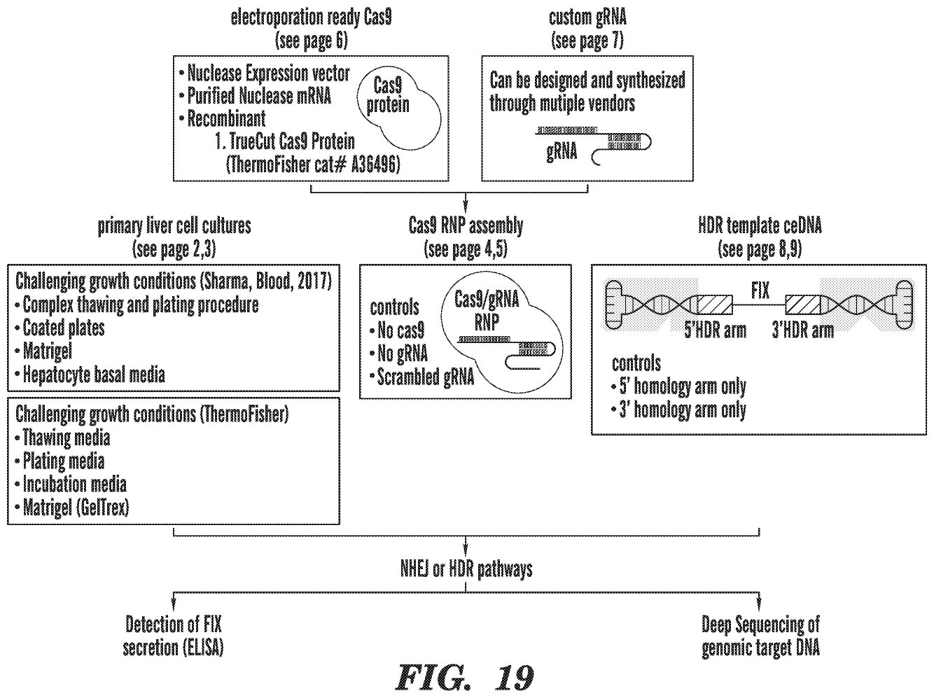

In another example, the methods depicted in FIG. 19 are used herein to perform gene editing in cultured cells. For example, any of the ceDNA vectors of the invention may be delivered through the methods described in the application and examples to a cultured cell, such as a liver cell culture. The cells are then incubated for a time and under conditions sufficient to effect gene editing using either the NHEJ pathway (in the case where the ceDNA vector does not comprise an HDR template) or via the HDR pathway (in the in the case where the ceDNA vector includes the HDR template). To determine if successful gene editing has occurred, the cells can be assayed for expression of the donor template protein, e.g., Factor IX (FIX), or by deep sequencing of the genomic target DNA to determine whether successful incorporation of the donor template has occurred. Further considerations with respect to this example are outlined below.

Design of Guide RNA:

Any method known in the art can be used to design and/or synthesize a custom guide RNA (gRNA) having homology to a target gene editing site for incorporation into a ceDNA vector of the invention. It is specifically contemplated herein that a custom gRNA can be designed and synthesized through multiple vendors, for example, ThermoFisher.

Primary Cell Cultures:

In some embodiments, it may be desired to target a gene editing site in a liver cell, such as a hepatocyte. This can be achieved, for example, by utilizing a gene editing site within the albumin gene. One of skill in the art will appreciate that successful isolation and growth of primary cells, including liver cells, can be challenging and may require optimization of thawing and plating procedures, coating of plates, Matrigel™, and/or growth media (e.g., hepatocyte basal media). In one embodiment, methods for culturing liver cells are derived from methods known in the art, for example, Sharma Blood (2017) (supra), the contents of which are incorporated herein by reference in its entirety. Growth conditions for primary liver cells can be optimized using reagents from e.g., ThermoFisher, such as thawing media, plating media, incubation media and matrix reagents (GelTrex™, MatriGel™)

HDR Template ceDNA:

In a nonlimiting example, the ceDNA vector comprising an HDR template is designed as shown in FIG. 19. For example, the ceDNA vector can comprise a 5′ homology arm having a desired length and a 3′ homology arm of a desired length. In order to rule out non-specific effects it is specifically contemplated herein that ceDNA controls comprising (i) the 5′ homology arm alone, with or without the donor template sequence, or (ii) the 3′ homology arm alone, with or without the donor template sequence, can be used in a substantially similar protocol as a ceDNA vector comprising the entire HDR template (e.g., 5′ homology arm, donor template sequence, and 3′ homology arm). These controls will permit one of skill in the art to discern non-specific or off-target effects, if any, that may be produced by the homology arms in isolation.

### Example 18: Exemplary Work-Flow Method(s) for Gene Editing In Vivo in a Subject

The methods and ceDNA constructs described in Example 17 can be adapted to perform gene editing in a multicellular organism, e.g., an animal or a human being. ceDNA vectors may be delivered into embryonic stem cells of the organism (e.g., mouse) in any convenient way. In some examples the organism is a non-human organism. An organism can be a rodent or animal (e.g., non-human primate) for the generation of an animal model of a disease. The resulting cells are screened to ensure the presence of the properly recombined transgene. The positive cells can be implanted into wild-type organisms (e.g., mice and non-human rodents), and the resulting offspring screened for presence of the transgene. Since the targeted mutations can be made in the gene of interest in any strain of the organism (e.g., mice), backcrossing of the offspring is not required to obtain transgenic offspring in the desired genetic background.

For illustrative purposes, this Example discusses using an exemplary gene editing ceDNA vector with respect to generating a ceDNA vector for editing cells to generate an animal model. However, while modification of animals (e.g., mouse) is exemplified in this Example to illustrate methods to use a gene editing vector ceDNA as described herein, one of ordinary skill in the art is aware that one can, as stated above use, use the ceDNA vector on cells from any organism or subject where gene editing is desired. Exemplary subjects for gene editing are discussed in the definition of “subject” herein.

### REFERENCES

All publications and references, including but not limited to patents and patent applications, cited in this specification and Examples herein are incorporated by reference in their entirety as if each individual publication or reference were specifically and individually indicated to be incorporated by reference herein as being fully set forth. Any patent application to which this application claims priority is also incorporated by reference herein in the manner described above for publications and references.

1. A non-viral capsid-free close-ended DNA (ceDNA) vector comprising:
at least one heterologous nucleotide sequence between flanking inverted terminal repeats (ITRs), wherein at least one heterologous nucleotide sequence encodes at least one gene editing molecule.

2. The ceDNA vector of claim 1, wherein at least one gene editing molecule is selected from a nuclease, a guide RNA (gRNA), a guide DNA (gDNA), and an activator RNA.

3. The ceDNA vector of claim 2, wherein at least one gene editing molecule is a nuclease.

4. The ceDNA vector of claim 3, wherein the nuclease is a sequence specific nuclease.

5. The ceDNA vector of claim 4, wherein the sequence specific nuclease is selected from a nucleic acid-guided nuclease, zinc finger nuclease (ZFN), a meganuclease, a transcription activator-like effector nuclease (TALEN), or a megaTAL.

6. The ceDNA vector of claim 5, wherein the sequence specific nuclease is a nucleic acid-guided nuclease selected from a single-base editor, an RNA-guided nuclease, and a DNA-guided nuclease.

7. The ceDNA vector of claim 2 or claim 6, wherein at least one gene editing molecule is a gRNA or a gDNA.

8. The ceDNA vector of claim 2, 6 or 7, wherein at least one gene editing molecule is an activator RNA.

9. The ceDNA of any one of claims 6-8, wherein the nucleic acid-guided nuclease is a CRISPR nuclease.

10. The ceDNA vector of claim 9, wherein the CRISPR nuclease is a Cas nuclease.

11. The ceDNA vector of claim 10, wherein the Cas nuclease is selected from Cas9, nicking Cas9 (nCas9), and deactivated Cas (dCas).

12. The ceDNA vector of claim 11, wherein the nCas9 contains a mutation in the HNH or RuVc domain of Cas.

13. The ceDNA vector of claim 11, wherein the Cas nuclease is a deactivated Cas nuclease (dCas) that complexes with a gRNA that targets a promoter region of a target gene.

14. The ceDNA vector of claim 13, further comprising a KRAB effector domain.

15. The ceDNA vector of claim 13 or claim 14, wherein the dCas is fused to a heterologous transcriptional activation domain that can be directed to a promoter region.

16. The ceDNA vector of claim 15, wherein the dCas fusion is directed to a promoter region of a target gene by a guide RNA that recruits additional transactivation domains to upregulate expression of the target gene.

17. The ceDNA vector of any one of claims 13-16, wherein the dCas is S. pyogenes dCas9.

18. The ceDNA vector of any one of claims 7-17, wherein the guide RNA sequence targets the promoter of a target gene and CRISPR silences the target gene (CRISPRi system).

19. The ceDNA vector of any one of claims 7-17, wherein the guide RNA sequence targets the transcriptional start site of a target gene and activates the target gene (CRISPRa system).

20. The ceDNA vector of any one of claims 6-19, wherein the at least one gene editing molecule comprises a first guide RNA and a second guide RNA.

21. The ceDNA vector of any one of claims 7-20, wherein the gRNA targets a splice acceptor or splice donor site.

22. The ceDNA vector of claim 21, wherein targeting the splice acceptor or splice donor site effects non-homologous end joining (NHEJ) and correction of a defective gene.

23. The ceDNA vector of any one of claims 7-22, wherein the vector encodes multiple copies of one guide RNA sequence.

24. The ceDNA vector of any one of claims 1-23, wherein a first heterologous nucleotide sequence comprises a first regulatory sequence operably linked to a nucleotide sequence that encodes a nuclease.

25. The ceDNA vector of claim 24, wherein the first regulatory sequence comprises a promoter.

26. The ceDNA vector of claim 25, wherein the promoter is CAG, Pol III, U6, or H1.

27. The ceDNA vector of any one of claims 24-26, wherein the first regulatory sequence comprises a modulator.

28. The ceDNA vector of claim 27, wherein the modulator is selected from an enhancer and a repressor.

29. The ceDNA vector of any one of claims 24-28, wherein the first heterologous nucleotide sequence comprises an intron sequence upstream of the nucleotide sequence that encodes the nuclease, wherein the intron sequence comprises a nuclease cleavage site.

30. The ceDNA vector of any one of claims 1-29, wherein a second heterologous nucleotide sequence comprises a second regulatory sequence operably linked to a nucleotide sequence that encodes a guide RNA.

31. The ceDNA vector of claim 30, wherein the second regulatory sequence comprises a promoter.

32. The ceDNA vector of claim 31, wherein the promoter is CAG, Pol III, U6, or H1.

33. The ceDNA vector of any one of claims 30-32, wherein the second regulatory sequence comprises a modulator.

34. The ceDNA vector of claim 33, wherein the modulator is selected from an enhancer and a repressor.

35. The ceDNA vector of any one of claims 1-34, wherein a third heterologous nucleotide sequence comprises a third regulatory sequence operably linked to a nucleotide sequence that encodes an activator RNA.

36. The ceDNA vector of claim 35, wherein the third regulatory sequence comprises a promoter.

37. The ceDNA vector of claim 36, wherein the promoter is CAG, Pol III, U6, or H1.

38. The ceDNA vector of any one of claims 35-37, wherein the third regulatory sequence comprises a modulator.

39. The ceDNA vector of claim 38, wherein the modulator is selected from an enhancer and a repressor.

40. The ceDNA vector of any one of claims 1-39, wherein the ceDNA vector comprises a 5′ homology arm and a 3′ homology arm to a target nucleic acid sequence.

41. The ceDNA vector of claim 40, wherein the 5′ homology arm and the 3′ homology arm are each between about 250 to 2000 bp.

42. The ceDNA vector of claim 40 or claim 41, wherein the 5′ homology arm and/or the 3′ homology arm are proximal to an ITR.

43. The ceDNA vector of any one of claims 40-42, wherein at least one heterologous nucleotide sequence is between the 5′ homology arm and the 3′ homology arm.

44. The ceDNA vector of claim 43, wherein the at least one heterologous nucleotide sequence that is between the 5′ homology arm and the 3′ homology arm comprises a target gene.

45. The ceDNA vector of any one of claims 40-44, wherein the ceDNA vector at least one heterologous nucleotide sequence that encodes a gene editing molecule is not between the 5′ homology arm and the 3′ homology arm.

46. The ceDNA vector of claim 45, wherein none of the heterologous nucleotide sequences that encode gene editing molecules are between the 5′ homology arm and the 3′ homology arm.

47. The ceDNA vector of any one of claims 40-46, comprising a first endonuclease restriction site upstream of the 5′ homology arm and/or a second endonuclease restriction site downstream of the 3′ homology arm.

48. The ceDNA vector of claim 47, wherein the first endonuclease restriction site and the second endonuclease restriction site are the same restriction endonuclease sites.

49. The ceDNA vector of claim 47 or claim 48, wherein at least one endonuclease restriction site is cleaved by an endonuclease which is also encoded on the ceDNA vector.

50. The ceDNA vector of any one of claims 40-49, wherein further comprises one or more poly-A sites.

51. The ceDNA vector of any one of claims 40-50, comprising at least one of a transgene regulatory element and a poly-A site downstream and proximate to the 3′ homology arm and/or upstream and proximate to the 5′ homology arm.

52. The ceDNA vector of any one of claims 40-51, comprising a 2A and selection marker site upstream and proximate to the 3′ homology arm.

53. The ceDNA vector of any one of claims 40-52, wherein the 5′ homology arm is homologous to a nucleotide sequence upstream of a nuclease cleavage site on a chromosome.

54. The ceDNA vector of any one of claims 40-53, wherein the 3′ homology arm is homologous to a nucleotide sequence downstream of a nuclease cleavage site on a chromosome.

55. The ceDNA vector of any one of claims 1-54, comprising a heterologous nucleotide sequence encoding an enhancer of homologous recombination.

56. The ceDNA vector of claim 55, wherein the enhancer of homologous recombination is selected from SV40 late polyA signal upstream enhancer sequence, the cytomegalovirus early enhancer element, an RSV enhancer, and a CMV enhancer.

57. The ceDNA vector of any one of claims 1-56, wherein at least one ITR comprises a functional terminal resolution site and a Rep binding site.

58. The ceDNA vector of any one of claims 1-57, wherein the flanking ITRs are symmetric or asymmetric.

59. The ceDNA vector of claim 58, wherein the flanking ITRs are asymmetric, wherein at least one of the ITRs is altered from a wild-type AAV ITR sequence by a deletion, addition, or substitution that affects the overall three-dimensional conformation of the ITR.

60. The ceDNA vector of any one of claims 1-59, wherein at least one heterologous nucleotide sequence is cDNA.

61. The ceDNA vector of claims 1-60, wherein one or more of the flanking ITRs are derived from an AAV serotype selected from AAV1, AAV2, AAV3, AAV4, AAV5, AAV6, AAV7, AAV8, AAV9, AAV10, AAV11, and AAV12.

62. The ceDNA vector of any one of claims 1-61, wherein one or more of the ITRs are synthetic.

63. The ceDNA vector of any one of claims 1-62, wherein one or more of the ITRs is not a wild type ITR.

64. The ceDNA vector of any one of claims 1-63, wherein one or more both of the ITRs is modified by a deletion, insertion, and/or substitution in at least one of the ITR regions selected from A, A′, B, B′, C, C′, D, and D′.

65. The ceDNA vector of claim 64, wherein the deletion, insertion, and/or substitution results in the deletion of all or part of a stem-loop structure normally formed by the A, A′, B, B′ C, or C′ regions.

66. The ceDNA vector of any one of claim 1-58 or 56-65, wherein the ITRs are symmetrical.

67. The ceDNA vector of any one of claims 1-58, 60, 61 and 66, wherein the ITRs are wild type.

68. The ceDNA vector of any one of claims 1-66, wherein both ITRs are altered in a manner that results in an overall three-dimensional symmetry when the ITRs are inverted relative to each other.

69. The ceDNA vector of claim 68, wherein the alteration is a deletion, insertion, and/or substitution in the ITR regions selected from A, A′, B, B′, C, C′, D, and D′.

70. A method for genome editing comprising:
contacting a cell with a gene editing system, wherein one or more components of the gene editing system are delivered to the cell by contacting the cell with a non-viral capsid-free close ended DNA (ceDNA) vector comprising at least one heterologous nucleotide sequence between flanking inverted terminal repeats (ITRs), wherein at least one heterologous nucleotide sequence encodes at least one gene editing molecule.

71. The method of claim 70, wherein at least one gene editing molecule is selected from a nuclease, a guide RNA (gRNA), a guide DNA (gDNA), and an activator RNA.

72. The method of claim 71, wherein at least one gene editing molecule is a nuclease.

73. The method of claim 72, wherein the nuclease is a sequence specific nuclease.

74. The method of claim 73, wherein the sequence specific nuclease is selected from a nucleic acid-guided nuclease, zinc finger nuclease (ZFN), a meganuclease, a transcription activator-like effector nuclease (TALEN), or a megaTAL.

75. The method of claim 73, wherein the sequence specific nuclease is a nucleic acid-guided nuclease selected from a single-base editor, an RNA-guided nuclease, and a DNA-guided nuclease.

76. The method of claim 70 or 75, wherein at least one gene editing molecule is a gRNA or a gDNA.

77. The method of claim 70, 75 or 76, wherein at least one gene editing molecule is an activator RNA.

78. The method of any one of methods 74-77, wherein the nucleic acid-guided nuclease is a CRISPR nuclease.

79. The method of claim 78, wherein the CRISPR nuclease is a Cas nuclease.

80. The method of claim 79, wherein the Cas nuclease is selected from Cas9, nicking Cas9 (nCas9), and deactivated Cas (dCas).

81. The method of claim 80, wherein the nCas9 contains a mutation in the HNH or RuVc domain of Cas.

82. The method of claim 80, wherein the Cas nuclease is a deactivated Cas nuclease (dCas) that complexes with a gRNA that targets a promoter region of a target gene.

83. The method of claim 82, further comprising a KRAB effector domain.

84. The method of claim 82 or 83, wherein the dCas is fused to a heterologous transcriptional activation domain that can be directed to a promoter region.

85. The method of claim 84, wherein the dCas fusion is directed to a promoter region of a target gene by a guide RNA that recruits additional transactivation domains to upregulate expression of the target gene.

86. The method of any of claims 82-85, wherein the dCas is S. pyogenes dCas9.

87. The method of any of claims 78-86, wherein the guide RNA sequence targets the promoter of a target gene and CRISPR silences the target gene (CRISPRi system).

88. The method of any of claims 78-86, wherein the guide RNA sequence targets the transcriptional start site of a target gene and activates the target gene (CRISPRa system).

89. The method of any of claims 76-88, wherein the at least one gene editing molecule comprises a first guide RNA and a second guide RNA.

90. The method of any of claims 76-89, wherein the gRNA targets a splice acceptor or splice donor site.

91. The method of claim 22, wherein targeting the splice acceptor or splice donor site effects non-homologous end joining (NHEJ) and correction of a defective gene.

92. The method of claim 76-91, wherein the vector encodes multiple copies of one guide RNA sequence.

93. The method of any of claims 70-92, wherein a first heterologous nucleotide sequence comprises a first regulatory sequence operably linked to a nucleotide sequence that encodes a nuclease.

94. The method of claim 93, wherein the first regulatory sequence comprises a promoter.

95. The method of claim 94, wherein the promoter is CAG, Pol III, U6, or H1.

96. The method of any of claims 93-95, wherein the first regulatory sequence comprises a modulator.

97. The method of claim 96, wherein the modulator is selected from an enhancer and a repressor.

98. The method of any of claims 93-97, wherein the first heterologous nucleotide sequence comprises an intron sequence upstream of the nucleotide sequence that encodes the nuclease, wherein the intron sequence comprises a nuclease cleavage site.

99. The method of any of claims 70-98, wherein a second heterologous nucleotide sequence comprises a second regulatory sequence operably linked to a nucleotide sequence that encodes a guide RNA.

100. The method of claim 99, wherein the second regulatory sequence comprises a promoter.

101. The method of claim 100, wherein the promoter is CAG, Pol III, U6, or H1.

102. The method of any of claims 99-101, wherein the second regulatory sequence comprises a modulator.

103. The method of claim 102, wherein the modulator is selected from an enhancer and a repressor.

104. The method of any of claims 70-103, wherein a third heterologous nucleotide sequence comprises a third regulatory sequence operably linked to a nucleotide sequence that encodes an activator RNA.

105. The method of claim 104, wherein the third regulatory sequence comprises a promoter.

106. The method of claim 105, wherein the promoter is CAG, Pol III, U6, or H1.

107. The method of claim 104-106, wherein the third regulatory sequence comprises a modulator.

108. The method of claim 107, wherein the modulator is selected from an enhancer and a repressor.

109. The method of any of claims 70-108, wherein the ceDNA vector comprises a 5′ homology arm and a 3′ homology arm to a target nucleic acid sequence.

110. The method of claim 109, wherein the 5′ homology arm and the 3′ homology arm are each between about 250 to 2000 bp.

111. The method of claim 109 or 110 wherein the 5′ homology arm and/or the 3′ homology arm are proximal to an ITR.

112. The method of any of claims 109-111, wherein at least one heterologous nucleotide sequence is between the 5′ homology arm and the 3′ homology arm.

113. The method of claim 112, wherein the at least one heterologous nucleotide sequence that is between the 5′ homology arm and the 3′ homology arm comprises a target gene.

114. The method of claim 109-113, wherein the ceDNA vector at least one heterologous nucleotide sequence that encodes a gene editing molecule is not between the 5′ homology arm and the 3′ homology arm.

115. The method of claim 114, wherein none of the heterologous nucleotide sequences that encode gene editing molecules are between the 5′ homology arm and the 3′ homology arm.

116. The method of claim 109-115, comprising a first endonuclease restriction site upstream of the 5′ homology arm and/or a second endonuclease restriction site downstream of the 3′ homology arm.

117. The method of claim 116, wherein the first endonuclease restriction site and the second endonuclease restriction site are the same restriction endonuclease sites.

118. The method of claim 116 or 117, wherein at least one endonuclease restriction site is cleaved by an endonuclease which is also encoded on the ceDNA vector.

119. The method of any of claims 109-118, wherein further comprises one or more poly-A sites.

120. The method of any of claims 109-119, comprising at least one of a transgene regulatory element and a poly-A site downstream and proximate to the 3′ homology arm and/or upstream and proximate to the 5′ homology arm.

121. The method of any of claims 109-120, comprising a 2A and selection marker site upstream and proximate to the 3′ homology arm.

122. The method of any of claims 109-121, wherein the 5′ homology arm is homologous to a nucleotide sequence upstream of a nuclease cleavage site on a chromosome.

123. The method of any of claims 109-122, wherein the 3′ homology arm is homologous to a nucleotide sequence downstream of a nuclease cleavage site on a chromosome.

124. The method of any of claims 109-123, comprising a heterologous nucleotide sequence encoding an enhancer of homologous recombination.

125. The method of claim 124, wherein the enhancer of homologous recombination is selected from SV40 late polyA signal upstream enhancer sequence, the cytomegalovirus early enhancer element, an RSV enhancer, and a CMV enhancer.

126. The method of any of claims 70-125, wherein at least one ITR comprises a functional terminal resolution site and a Rep binding site.

127. The method of any of claims 70-126, wherein the flanking ITRs are symmetric or asymmetric.

128. The method of claim 127, wherein the flanking ITRs are asymmetric, wherein at least one of the ITRs is altered from a wild-type AAV ITR sequence by a deletion, addition, or substitution that affects the overall three-dimensional conformation of the ITR.

129. The method of any of claims 70-128, wherein at least one heterologous nucleotide sequence is cDNA.

130. The method of any of claims 70-129, wherein one or more of the flanking ITRs are derived from an AAV serotype selected from AAV1, AAV2, AAV3, AAV4, AAV5, AAV6, AAV7, AAV8, AAV9, AAV10, AAV11, and AAV12.

131. The method of any of claims 70-130, wherein one or more of the ITRs are synthetic.

132. The method of any of claims 70-131, wherein one or more of the ITRs is not a wild type ITR.

133. The method of any of claims 70-132, wherein one or more both of the ITRs is modified by a deletion, insertion, and/or substitution in at least one of the ITR regions selected from A, A′, B, B′, C, C′, D, and D′.

134. The method of claim 133, wherein the deletion, insertion, and/or substitution results in the deletion of all or part of a stem-loop structure normally formed by the A, A′, B, B′ C, or C′ regions.

135. The method of any of claim 70-127 or 129-134, wherein the ITRs are symmetrical.

136. The method of any one of claim 70-127 or 129-130, wherein the ITRs are wild type.

137. The method of any of claims 70-136, wherein both ITRs are altered in a manner that results in an overall three-dimensional symmetry when the ITRs are inverted relative to each other.

138. The method of claim 137, wherein the alteration is a deletion, insertion, and/or substitution in the ITR regions selected from A, A′, B, B′, C, C′, D, and D′.

139. The method of any of claims 70-138, wherein the cell contacted is a eukaryotic cell.

140. The method of any of claims 84-139, wherein the CRISPR nuclease is codon optimized for expression in the eukaryotic cell.

141. The method of any of claims 84-140, wherein the Cas protein is codon optimized for expression in the eukaryotic cell.

142. A method of genome editing comprising administering to a cell an effective amount of a non-viral capsid-free closed ended DNA (ceDNA vector) of any one of claims 1-69, under conditions suitable and for a time sufficient to edit a target gene.

143. The method of any of claims 113-142, wherein the target gene is gene targeted using one or more guide RNA sequences and edited by homology directed repair (HDR) in the presence of a HDR donor template.

144. The method of any of claims 142-143, wherein the target gene is targeted using one guide RNA sequence and the target gene is edited by non-homologous end joining (NHEJ).

145. The method of any of claims 70-144, wherein the method is performed in vivo to correct a single nucleotide polymorphism (SNP) associated with a disease.

146. The method of claim 145, wherein the disease comprises sickle cell anemia, hereditary hemochromatosis or cancer hereditary blindness.

147. The method of any of claims 70-146, wherein at least 2 different Cas proteins are present in the ceDNA vector, and wherein one of the Cas protein is catalytically inactive (Cas-i), and wherein the guide RNA associated with the Cas-I targets the promoter of the target cell, and wherein the DNA coding for the Cas-I is under the control of an inducible promoter so that it can turn-off the expression of the target gene at a desired time.

148. A method for editing a single nucleotide base pair in a target gene of a cell, the method comprising contacting a cell with a CRISPR/Cas gene editing system, wherein one or more components of the CRISPR/Cas gene editing system are delivered to the cell by contacting the cell with a non-viral capsid-free close-ended DNA (ceDNA) vector composition, and
wherein the Cas protein expressed from the ceDNA vector is catalytically inactive and is fused to a base editing moiety,
wherein the method is performed under conditions and for a time sufficient to modulate expression of the target gene.

149. The method of claim 148, wherein the ceDNA vector is a ceDNA vector of any of claims 1-69.

150. The method of claim 148, wherein the base editing moiety comprises a single-strand-specific cytidine deaminase, a uracil glycosylase inhibitor, or a tRNA adenosine deaminase.

151. The method of claim 148, wherein the catalytically inactive Cas protein is dCas9.

152. The method of any of claims 70-151, wherein the cell is a T cell, or CD34⁺.

153. The method of any of claims 70-152, wherein the target gene encodes for a programmed death protein (PD1), cytotoxic T-lymphocyte-associated antigen 4 (CTLA4), or tumor necrosis factor-α (TNF-α).

154. The method of any of claims 70-153, further comprising administering the cells produced to a subject in need thereof.

155. The method of claim 154, wherein the subject in need thereof has a genetic disease, viral infection, bacterial infection, cancer, or autoimmune disease.

156. A method of modulating expression of two or more target genes in a cell comprising: introducing into the cell:
(iv) a first composition comprising a vector that comprises: flanking terminal repeat (TR) sequences, and a nucleic acid sequence encoding at least two guide RNAs complementary to two or more target genes, wherein the vector is a non-viral capsid free closed ended DNA (ceDNA) vector,
(v) a second composition comprising a vector that comprises: flanking terminal repeat (TR) sequences and a nucleic acid sequence encoding at least two catalytically inactive DNA endonucleases that each associate with a guide RNA and bind to the two or more target genes, wherein the vector is a non-viral capsid free closed ended DNA (ceDNA) vector, and
(vi) a third composition comprising a vector that comprises: flanking terminal repeat (TR) sequences, and a nucleic acid sequence encoding at least two transcriptional regulator proteins or domains, wherein the vector is a non-viral capsid free closed ended DNA (ceDNA) vector and
wherein the at least two guide RNAs, the at least two catalytically inactive RNA-guided endonucleases and the at least two transcriptional regulator proteins or domains are expressed in the cell,
wherein two or more co-localization complexes form between a guide RNA, a catalytically inactive RNA-guided endonuclease, a transcriptional regulator protein or domain and a target gene, and
wherein the transcriptional regulator protein or domain regulates expression of the at least two target genes.

157. The method of claim 156, wherein the ceDNA vector of the first composition is a ceDNA vector of any of claims 1-69, the ceDNA vector of the second composition is a ceDNA vector of any of claims 1-69, and the third composition is a ceDNA vector of any of claims 1-69.

158. A method for inserting a nucleic acid sequence into a genomic safe harbor gene, the method comprising: contacting a cell with (i) a gene editing system and (ii) a homology directed repair template having homology to a genomic safe harbor gene and comprising a nucleic acid sequence encoding a protein of interest,
wherein one or more components of the gene editing system are delivered to the cell by contacting the cell with a non-viral capsid-free close-ended DNA (ceDNA) vector composition, wherein the ceDNA nucleic acid vector composition comprises at least one heterologous nucleotide sequence between flanking inverted terminal repeats (ITRs), wherein at least one heterologous nucleotide sequence encodes at least one gene editing molecule, and
wherein the method is performed under conditions and for a time sufficient to insert the nucleic acid sequence encoding the protein of interest into the genomic safe harbor gene.

159. The method of claim 158, wherein the ceDNA vector is a ceDNA vector of any of claims 1-69.

160. The method of claim 158, wherein the genomic safe harbor gene comprises an active intron close to at least one coding sequence known to express proteins at a high expression level.

161. The method of claim 158, wherein the genomic safe harbor gene comprises a site in or near any one of: the albumin gene, CCR5 gene, AAVS1 locus.

162. The method of any of claims 158-161, wherein the protein of interest is a receptor, a toxin, a hormone, an enzyme, or a cell surface protein.

163. The method of any of claim 162, wherein, the protein of interest is a secreted protein.

164. The method of claim 163, wherein the protein of interest comprises Factor VIII (FVIII) or Factor IX (FIX).

165. The method of claim 164, wherein the method is performed in vivo for the treatment of hemophilia A, or hemophilia B.

166. A method of inserting a donor sequence at a predetermined insertion site on a chromosome in a host cell, comprising: introducing into the host cell the ceDNA vector of claims 1-69, wherein the donor sequence is inserted into the chromosome at or adjacent to the insertion site through homologous recombination.

167. A method of generating a genetically modified animal comprising a donor sequence inserted at a predetermined insertion site on the chromosome of the animal, comprising a) generating a cell with the donor sequence inserted at the predetermined insertion site on the chromosome according to claim 167; and b) introducing the cell generated by a) into a carrier animal to produce the genetically modified animal.

168. The method of claim 167, wherein the cell is a zygote or a pluripotent stem cell.

169. A genetically modified animal generated by the method of claim 168.

170. The genetically modified animal of claim 169, wherein the animal is a non-human animal.

171. A kit for inserting a donor sequence at an insertion site on a chromosome in a cell, comprising: a) a first non-viral capsid-free close-ended DNA (ceDNA) vector comprising:
two AAV inverted terminal repeat (ITR); and
a first nucleotide sequence comprising a 5′ homology arm, a donor sequence, and a 3′ homology arm, wherein the donor sequence has gene editing functionality; and
(b) a second ceDNA vector comprising:
at least one AAV ITR; and
a nucleotide sequence encoding at least one gene editing molecule,
wherein in the first ceDNA vector, the 5′ homology arm is homologous to a sequence upstream of a cleavage site for gene editing molecule on the chromosome and wherein the 3′ homology arm is homologous to a sequence downstream of the gene editing molecule cleavage site on the chromosome; and wherein the 5′ homology arm or the 3′ homology arm are proximal to the ITR.

172. The method of claim 171, wherein the gene editing molecule is a nuclease.

173. The method of claim 172, wherein the nuclease is a sequence specific nuclease.

174. The method of any of claims 171-173, wherein the first ceDNA vector is a ceDNA vector of any of claims 1, 40-56, 57-69.

175. The method of any of claims 171-173, wherein the second ceDNA vector is a ceDNA vector of any of claims 1-39 or claims 57-69.

176. A method of inserting a donor sequence at a predetermined insertion site on a chromosome in a host cell, comprising:
a) introducing into the host cell a first non-viral capsid-free close-ended DNA (ceDNA) vector having at least one inverted terminal repeat (ITR), wherein the ceDNA vector comprises a first linear nucleic acid comprising a 5′ homology arm, a donor sequence, and a 3′ homology arm; and
b) introducing into the host cell a second ceDNA vector comprising least one heterologous nucleotide sequence between flanking inverted terminal repeats (ITRs), wherein at least one heterologous nucleotide sequence encodes at least one gene editing molecule that cleaves the chromosome at or adjacent to the insertion site, wherein the donor sequence is inserted into the chromosome at or adjacent to the insertion site through homologous recombination.

177. The method of claim 176, wherein the gene editing molecule is a nuclease.

178. The method of claim 177, wherein the nuclease is a sequence specific nuclease.

179. The method of any of claims 176-178, wherein the first ceDNA vector is a ceDNA vector of any of claims 1, 40-56, 57-69.

180. The method of any of claims 176-179, wherein the second ceDNA vector is a ceDNA vector of any of claims 1-39 or claims 57-69.

181. The method of any of claims 179-180, wherein the second ceDNA vector further comprises a third nucleotide sequence encoding a guide sequence recognizing the insertion site.

182. A cell containing a ceDNA vector of any of claims 1-69.

183. A composition comprising a vector of any of claims 1-69 and a lipid.

184. The composition of claim 184, wherein the lipid is a lipid nanoparticle (LNP).

185. A kit comprising a composition of claim 183 or 184 or a cell of claim 182.
